(19) United States
(12) Patent Application Publication
(10) Pub. No.: US 2026/0366028 A1
(43) Pub. Date: Nov. 19, 2026

(54) PEPTIDE-ANTISENSE OLIGONUCLEOTIDES AND THEIR USE FOR TREATMENT OF NEURODEGENERATIVE DISORDERS

(71) Applicant: EISAI R&D MANAGEMENT CO., LTD., Tokyo (JP)

(72) Inventors: Vinod VATHIPADIEKAL, Cambridge, MA (US); Branko MITASEV, Cambridge, MA (US); John WANG, Cambridge, MA (US); Courtney EASLEY-NEAL, Cambridge, MA (US); Hyeong Wook CHOI, Cambridge, MA (US); Francis G. FANG, Cambridge, MA (US); Praveen VEMULA, Cambridge, MA (US); Jung Hwa LEE, Cambridge, MA (US); Jeffrey HENRY, Cambridge, MA (US)

(21) Appl. No.: 19/124,226

(22) PCT Filed: Oct. 27, 2023
(86) PCT No.: PCT/US2023/078121

§ 371 Date: Apr. 24, 2025

Related U.S. Application Data
(60) Provisional application No. 63/420,027, filed on Oct. 27, 2022.

(51) Int. Cl. C12N 15/113; A61K 47/64; A61P 25/28; C07K 7/64

(52) U.S. Cl. CPC C12N 15/1138; A61K 47/645; A61P 25/28; C07K 7/64; C07K 2319/09; C07K 2319/10; C12N 2310/11; C12N 2310/314; C12N 2310/3233; C12N 2310/3513; C12N 2320/33

(57) ABSTRACT

Novel conjugates of antisense oligonucleotides and cell-penetrating peptides that induce Exon-2 skipping in the CD33 gene during pr-mRNA splicing, and their use in the treatment of a neurodegenerative disease, such as Alzheimer's disease, are disclosed.

### RELATED APPLICATION

The present application claims the benefit of priority to U.S. Provisional Patent Application No. 63/420,027, filed Oct. 27, 2022, which is incorporated herein by reference in its entirety.

### FIELD

Disclosed herein are novel peptide-antisense oligonucleotides comprising cell penetrating peptides conjugated to antisense oligonucleotides (“CPP-ASOs”) that may induce exon skipping during pre-mRNA splicing, pharmaceutical compositions comprising the same, and methods of using the same.

### BACKGROUND

Neurodegenerative disorders are a group of disorders characterized by the decline of central nervous system and peripheral nervous system structure and function. While neurodegenerative disorders exhibit heterogeneous symptoms, they can share similar features. One neurodegenerative disease, Alzheimer's Disease, is a neurodegenerative disorder characterized by buildup of amyloid beta plaques and neurofibrillary tangles. It is also the leading cause of dementia. Although some cases of rare familial Alzheimer's Disease involve autosomal dominant mutations to the amyloid beta precursor protein, the majority of cases are late-onset Alzheimer's Disease (LOAD), which do not follow Mendelian inheritance patterns. While the mechanics of LOAD are not completely understood, genome-wide association studies have identified genetic risk factors for LOAD. Scientists have shown the ability of these genes to impact the production, aggregation, or clearance of amyloid beta plaques. One such gene is CD33, also known as Siglec-3. Griciuc et al., Alzheimer's Disease Risk Gene CD33 Inhibits Microglial Uptake of Amyloid Beta, 78 NEURON 631 (2013).

CD33 is expressed in myeloid-derived cells, including macrophages such as microglia, and encodes the CD33 protein. Microglia account for approximately 10% of the cells in the brain and represent the first line of immunological defense. Microglia modulate several important activities in the brain, such as homeostasis, cognition, and neurogenesis. Augusto-Oliveira et al., What Do Microglia Really Do in Healthy Adult Brain?, 8 CELLS 1293 (2019). Microglia cells are known to contribute to neurodegeneration by releasing proinflammatory substances in the central nervous system. Wojtera et al., Microglial cells in neurodegenerative disorders, 43 FOLIA NEUROPATHOLOGY 311 (2005).

CD33 is a transmembrane receptor protein that has an extracellular receptor that binds the ligand sialic acid. The intracellular immunoreceptor tyrosine-based inhibition motif recruits phosphatases upon phosphorylation of its tyrosine residues, leading to suppression of immune cell activity such as phagocytosis. CD33 has been found to inhibit microglial uptake of amyloid beta protein, which suggests that therapies targeting CD33 could be potential LOAD treatment options. Griciuc et al., Alzheimer's Disease Risk Gene CD33 Inhibits Microglial Uptake of Amyloid Beta, 78 NEURON 631 (2013).

Two single nucleotide polymorphisms (SNPs) in the promoter region of the CD33 gene are associated with LOAD: rs3826656 and rs3865444. The rs3865444 SNP comes in two forms, rs3865444-C and rs3865444-A. The first form results in normal length CD33 protein. The second form, rs3865444-A, modulates splicing of CD33 pre-mRNA, resulting in skipping of Exon-2 and a CD33 protein lacking the sialic acid binding domain. Malik et al., CD33 Alzheimer's Risk-Altering Polymorphism, CD33 Expression, and Exon 2 Splicing, 33 J. NEUROSCIENCE 13320 (2013).

In eukaryotic genes containing coding (exons) and noncoding (intron) sequences, the noncoding introns are excised from the pre-mRNA transcript and the coding exons are spliced together to form mRNA. If an intron is left in the final mRNA transcript or an exon is left out, the mRNA reading frame may be disrupted during translation of the mRNA. This may result in a non-functional polypeptide sequence or a premature stop codon. The splicing process is further complicated by alternative splicing, where the same pre-mRNA sequence can be spliced into different exon combinations to form multiple mRNA sequences.

Splicing of pre-mRNA is an intricate process involving a multi-megadalton ribonucleoprotein complex called the spliceosome. The spliceosome recognizes specific sequences in pre-mRNA to precisely excise introns and ligate exons. The spliceosome catalyzes intron excision in two transesterification reactions using three conserved RNA sequences. These RNA sequences are the 5′ splice site, 3′ splice site, and the branch site. Wil & Luhrmann, Spliceosome Structure and Function, 3 COLD SPRING HARB. PERSPECT. BIOL. 1 (2011).

Splicing begins with the 2′ OH group of the branch site binding to the 5′ splice site via a nucleophilic attack, causing cleavage of the 5′ exon at the 5′ splice site and forming a lariat. Then the 3′ OH group of the 5′ exon attacks the 3′ exon at the 3′ splice site, ligating the 5′ and 3′ exons and cleaving the intron lariat. WII & Luhrmann, Spliceosome Structure and Function, 3 COLD SPRING HARB. PERSPECT. BIOL. 1 (2011). Because the splicing process involves spliceosome recognition sites, 5′ and 3′ splice sites, and the branch site, a mutation in any one of these sites can disrupt the splicing process.

ASOs are polynucleotides designed to bind with specificity to a target nucleotide sequence, thereby affecting one or more aspects of gene expression, such as transcription, splicing, stability, and/or translation. ASOs may be directed to either RNA or DNA. ASOs directed to RNA can bind to target mRNA sequences, affecting mRNA stability or translation at the ribosome.

ASOs that bind to target sequences in pre-mRNA transcripts can affect the splicing process. In some cases, ASOs may be used to induce exon skipping during pre-mRNA splicing. For example, Duchenne Muscular Dystrophy (DMD) is caused by a mutation that alters the reading frame of dystrophin mRNA during translation, resulting in a premature stop codon and truncated dystrophin protein. ASOs may be utilized to correct the reading frame by inducing skipping of an exon during splicing. Removing an exon of the correct number of base pairs results in a shorter mRNA transcript, but the reading frame may be corrected. Because dystrophin RNA consists of 79 exons, skipping one or several exons during splicing still results in a partly functional protein. Echigoya et al., Multiple Exon Skipping in the Duchenne Muscular Dystrophy Hot Spots: Prospects and Challenges, 8 J. PERS. MED. 41 (2018). The FDA approved an exon-skipping drug called Exondys 51 (eteplirsen) for treatment of DMD in 2016. Dowling, Eteplirsen therapy for Duchenne muscular dystrophy: skipping to the front of the line, 12 NATURE REV. NEUROLOGY 675 (2016).

In other cases, ASOs may be used to prevent or reduce exon skipping during pre-mRNA splicing. As an example, the ASO drug nusinersen (Spinraza®) reduces Exon-7 skipping during splicing of the SMN2 gene to treat spinal muscular atrophy. Son & Yokota, Recent Advances and Clinical Applications of Exon Inclusion for Spinal Muscular Atrophy, in EXON SKIPPING & INCLUSION THERAPIES, 57-68 (2018). The rs3865444-A variant that induces Exon-2 skipping of CD33 conveys protection against LOAD. Malik et al., CD33 Alzheimer's Risk-Altering Polymorphism, CD33 Expression, and Exon 2 Splicing, 33 J. NEUROSCIENCE 13320 (2013). There remains a need, however, for ASOs that successfully induce Exon-2 skipping during pre-mRNA splicing of CD33 and for their use in treating neurodegenerative diseases.

### SUMMARY OF THE INVENTION

Disclosed herein are CPP-ASOs, methods of using such CPP-ASOs to induce exon skipping during pre-mRNA splicing, pharmaceutical compositions that comprise such CPP-ASOs, and methods of using such compositions to treat neurodegenerative disease.

In some embodiments, disclosed herein is a peptide-antisense oligonucleotide conjugate, comprising a cell-penetrating peptide conjugated to an antisense oligonucleotide, wherein the antisense oligonucleotide is complementary to a portion of SEQ ID NO:1, and wherein the peptide-antisense oligonucleotide conjugate has a CD33 Exon-2 skipping efficiency of 30% or greater according to a Standard Exon-Skipping Efficiency Assay for the antisense oligonucleotide. In some embodiments, the peptide-antisense oligonucleotide conjugate comprises a cell-penetrating peptide conjugated to an antisense oligonucleotide, wherein the antisense oligonucleotide comprises all or a portion of SEQ ID NO:2, SEQ ID NO:12, or SEQ ID NO:224. In some embodiments, the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length. In some embodiments, the antisense oligonucleotide is 21-30 nucleotides in length. In some embodiments, the antisense oligonucleotide is 21-25 nucleotides in length. In some embodiments, the antisense oligonucleotide is 18-21 nucleotides in length. In some embodiments, the antisense oligonucleotide is 18-25 nucleotides in length. In some embodiments, the antisense oligonucleotide is 25-30 nucleotides in length. In some embodiments, the antisense oligonucleotide is 21 or 25 nucleotides in length. In some embodiments, the antisense oligonucleotide is 25 nucleotides in length.

In some embodiments, disclosed herein is a peptide-antisense oligonucleotide conjugate, wherein the antisense oligonucleotide comprises one or more non-natural sugar moieties, one or more non-natural internucleotide linkages, or one or more non-natural sugar moieties and one or more non-natural internucleotide linkages. In some embodiments, the antisense oligonucleotide comprises one or more non-natural sugar moieties. In some embodiments, the antisense oligonucleotide comprises a phosphorodiamidate morpholino oligomer (PMO). In some embodiments, the antisense oligonucleotide has a CD33 Exon-2 skipping efficiency of 30% or greater according to a Standard Exon-Skipping Efficiency Assay for PMO ASOs. In some embodiments, the antisense oligonucleotide comprises a methoxyethyl ribose oligomer (MOE). In some embodiments, the antisense oligonucleotide has a CD33 Exon-2 skipping efficiency of 30% or greater according to a Standard Exon-Skipping Efficiency Assay for MOE ASOs. In some embodiments, the antisense oligonucleotide comprises one or more non-natural internucleotide linkages. In some embodiments, the one or more non-natural internucleotide linkages comprise one or more phosphorodiamidate linkages and/or one or more phosphorothioate linkages. In some embodiments, all of the one or more non-natural internucleotide linkages have an Sp configuration. In some embodiments, all of the one or more non-natural internucleotide linkages have an Rp configuration. In some embodiments, the antisense oligonucleotide comprises one or more non-natural internucleotide linkages having an Sp configuration and one or more non-natural internucleotide linkages having an Rp configuration. In some embodiments, the antisense oligonucleotide comprises one or more modified nucleobases.

In some embodiments, disclosed herein is a composition comprising a peptide-antisense oligonucleotide conjugate and optionally a pharmaceutically acceptable carrier or excipient.

In some embodiments, disclosed herein is a peptide-antisense oligonucleotide conjugate comprising a cell-penetrating peptide conjugated to all or a portion of PMO-002 (SEQ ID NO:2), MOE-012 (SEQ ID NO:12), or PMO-424 (SEQ ID NO:224). In some embodiments, the peptide-antisense oligonucleotide conjugate comprises a cell-penetrating peptide conjugated to an antisense oligonucleotide selected from the group consisting of PMO-002 (SEQ ID NO:2), MOE-012 (SEQ ID NO:12), and PMO-424 (SEQ ID NO:224). In some embodiments, the peptide comprises at least one proteogenic amino acid, at least one non-proteogenic amino acid, or at least one proteogenic amino acid and at least one non-proteogenic amino acid. In some embodiments, the peptide comprises 5-25 amino acids. In some embodiments, the non-proteogenic amino acid comprises a modified proline residue, a lipophilic group, and/or a lactam group. In some embodiments, the peptide is a linear peptide. In some embodiments, the linear peptide comprises at least a portion of Pip6a, ApoE, and/or a neurotensin-based peptide. In some embodiments, the peptide is a cyclic peptide. In some embodiments, the cyclic peptide is CPP9

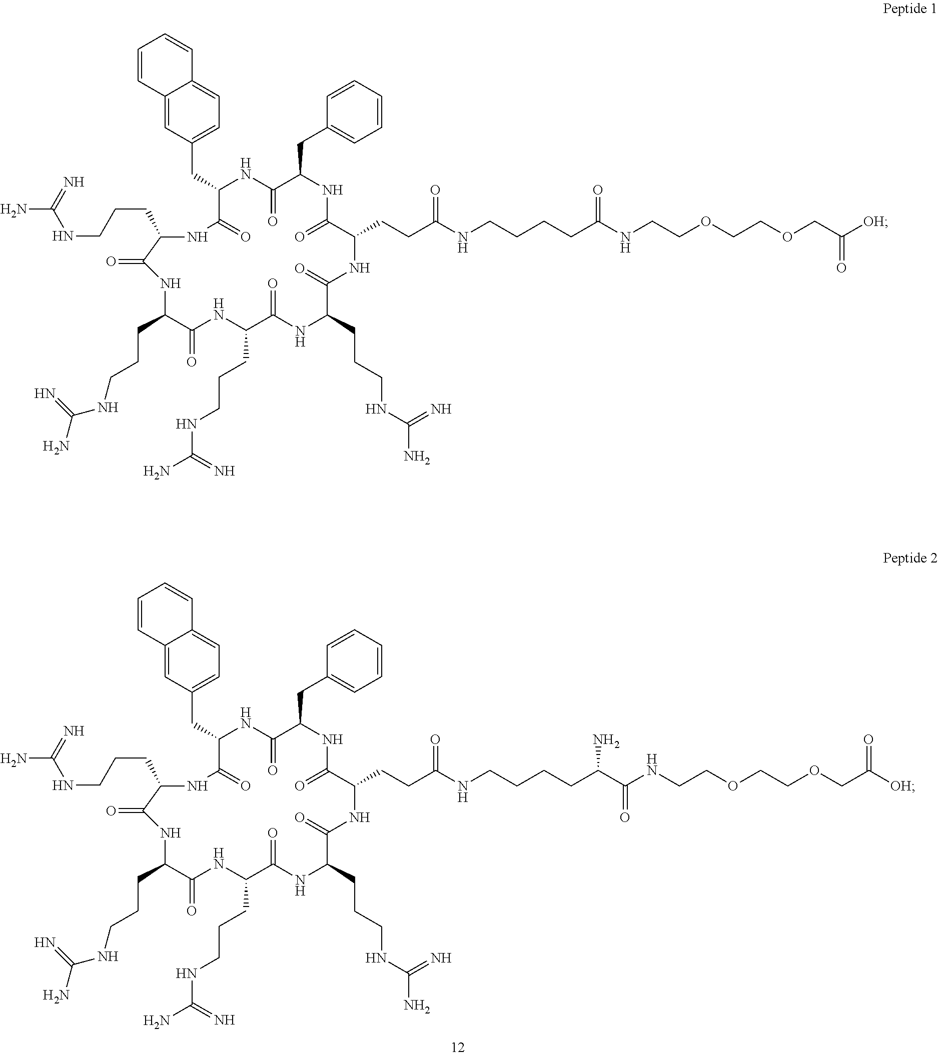 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O ; N=C(N)NCCC[C@@H]1NC(=O)[C@H](Cc2ccc3ccccc3c2)NC(=O)[C@@H](Cc2ccccc2)NC(=O)[C@H](CCC(=O)NCCCC[C@H](N)C(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

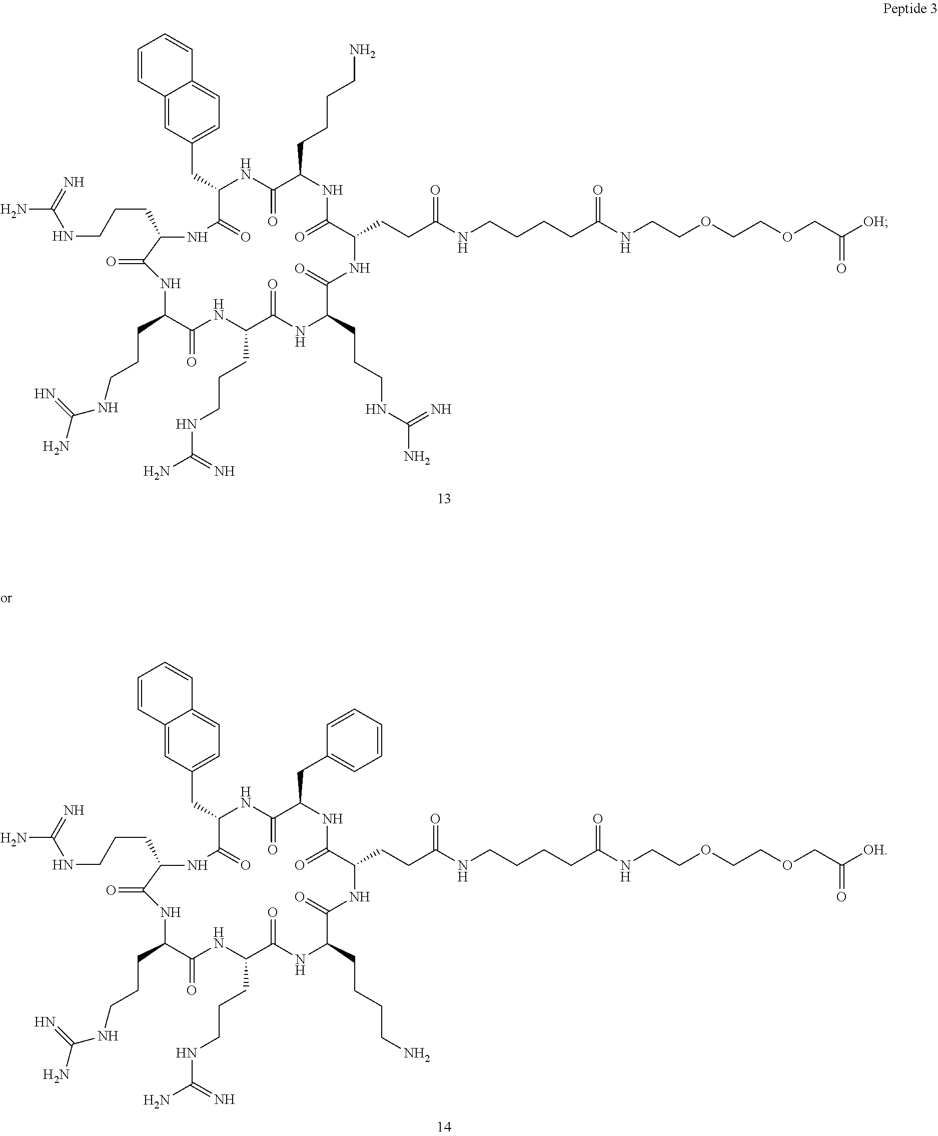 [SMILES: N=C(N)NCCC[C@@H]1NC(=O)[C@H](Cc2ccc3ccccc3c2)NC(=O)[C@@H](CCCCN)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O ; N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCCN)NC(=O)[C@H](CCCNC(=N)N)NC(=O)[C@@H](CCCNC(=N)N)NC1=O]

In Some embodiments, the peptide Comprises a lipoic acid group. In Some embodiments, the lipoic acid group is an (R)-lipoic acid group. In some embodiments, the lipoic acid group is an (S)-lipoic acid group. In some embodiments, the peptide comprises an oxadiazole linkage. In some embodiments, the peptide is conjugated directly to the antisense oligonucleotide. In some embodiments, the peptide is conjugated to the antisense oligonucleotide using a chemical reaction. In some embodiments, the chemical reaction is a strain-promoted azide-alkyne cycloaddition reaction, a strained alkene-tetrazine cycloaddition reaction, or an amide bond reaction.

In some embodiments, the peptide is indirectly Conjugated to the antisense oligonucleotide, wherein a linker is conjugated between the peptide and antisense oligonucleotide. In some embodiments, the peptide-antisense oligonucleotide conjugate further comprises one or more nuclear localization sequences, wherein the one or more nuclear localization sequences are independently conjugated to the peptide and/or the antisense oligonucleotide.

In some embodiments, disclosed here in is a method of inducing Exon-2 skipping in the CD33 gene during pre-mRNA splicing, comprising introducing a peptide-antisense oligonucleotide conjugate into a cell, wherein the peptide-antisense oligonucleotide conjugate comprises a cell-penetrating peptide conjugated to an antisense oligonucleotide, wherein the antisense oligonucleotide is complementary to a portion of SEQ ID NO:1, and wherein the peptide-antisense oligonucleotide conjugate has a CD33 Exon-2 skipping efficiency of 30% or greater according to a Standard Exon-Skipping Efficiency Assay for the antisense oligonucleotide. In some embodiments, the peptide-antisense oligonucleotide conjugate comprises a cell-penetrating peptide conjugated to an antisense oligonucleotide, and wherein the antisense oligonucleotide comprises all or a portion of SEQ ID NO:2, SEQ ID NO:12, or SEQ ID NO:224. In some embodiments, the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length. In some embodiments, the antisense oligonucleotide comprises one or more non-natural sugar moieties, one or more non-natural internucleotide linkages, or one or more non-natural sugar moieties and one or more non-natural internucleotide linkages. In some embodiments, the antisense oligonucleotide comprises one or more non-natural sugar moieties. In some embodiments, the antisense oligonucleotide comprises a phosphorodiamidate morpholino oligomer (PMO). In some embodiments, the peptide-antisense oligonucleotide has a CD33 Exon-2 skipping efficiency of 30% or greater according to a Standard Exon-Skipping Efficiency Assay for PMO ASOs. In some embodiments, the antisense oligonucleotide comprises a methoxyethyl ribose oligomer (MOE). In some embodiments, the antisense oligonucleotide has a CD33 Exon-2 skipping efficiency of 30% or greater according to a Standard Exon-Skipping Efficiency Assay for MOE ASOs. In some embodiments, the antisense oligonucleotide comprises one or more non-natural internucleotide linkages. In some embodiments, all of the one or more non-natural internucleotide linkages have an Sp configuration In some embodiments, all of the one or more non-natural internucleotide linkages have an Rp configuration. In some embodiments, the one or more non-natural internucleotide linkages comprise one or more non-natural internucleotide linkages having an Sp configuration and one or more non-natural internucleotide linkages having an Rp configuration. In some embodiments, the antisense oligonucleotide comprises one or more modified nucleobases. In some embodiments, the peptide-antisense oligonucleotide further comprises a pharmaceutically acceptable carrier or excipient.

In some embodiments, disclosed herein is a method of inducing Exon-2 skipping in the CD33 gene during pre-mRNA splicing, comprising introducing a peptide-antisense oligonucleotide conjugate into a cell, wherein the peptide-antisense oligonucleotide conjugate comprises an antisense oligonucleotide conjugated to a cell-penetrating peptide, and wherein the antisense oligonucleotide comprises all or a portion of PMO-002 (SEQ ID NO:2), MOE-012 (SEQ ID NO:12) or PMO-424 (SEQ ID NO:224). In some embodiments, the cell is an animal cell. In some embodiments, the cell is a human cell.

In some embodiments, disclosed herein is a method of treating a subject having a neurodegenerative disease comprising administering to said subject a therapeutically effective amount of a peptide-antisense oligonucleotide conjugate, wherein the peptide-antisense oligonucleotide conjugate comprises an antisense nucleotide conjugated to a cell-penetrating peptide, wherein the antisense oligonucleotide is complementary to a portion of SEQ ID NO:1, and wherein the peptide-antisense oligonucleotide conjugate has a CD33 Exon-2 skipping efficiency of 30% or greater according to a Standard Exon-Skipping Efficiency Assay for the antisense oligonucleotide. In some embodiments, the antisense oligonucleotide is complementary to all or a portion of SEQ ID NO:2, SEQ ID NO:12 or SEQ ID NO:224. In some embodiments, the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length. In some embodiments, the antisense oligonucleotide comprises one or more non-natural sugar moieties, one or more non-natural internucleotide linkages, or one or more non-natural sugar moieties and one or more non-natural internucleotide linkages. In some embodiments, the antisense oligonucleotide comprises one or more non-natural sugar moieties. In some embodiments, the antisense oligonucleotide comprises a phosphorodiamidate morpholino oligomer (PMO). In some embodiments, the peptide-antisense oligonucleotide has a CD33 Exon-2 skipping efficiency of 30% or greater according to a Standard Exon-Skipping Efficiency Assay for PMO ASOs. In some embodiments, the antisense oligonucleotide comprises a methoxyethyl ribose oligomer (MOE). In some embodiments, the antisense oligonucleotide has a CD33 Exon-2 skipping efficiency of 30% or greater according to a Standard Exon-Skipping Efficiency Assay for MOE ASOs. In some embodiments, the antisense oligonucleotide comprises one or more non-natural internucleotide linkages. In some embodiments, all of the one or more non-natural internucleotide linkages have an Sp configuration. In some embodiments, all of the one or more non-natural internucleotide linkages have an Rp configuration. In some embodiments, the one or more non-natural internucleotide linkages comprise one or more non-natural internucleotide linkages having an Sp configuration and one or more non-natural internucleotide linkages having an Rp configuration. In some embodiments, the antisense oligonucleotide comprises one or more modified nucleobases. In some embodiments, the antisense oligonucleotide further comprises a pharmaceutically acceptable carrier or excipient.

In some embodiments, disclosed herein is a method of treating a subject having a neurodegenerative disease comprising administering to said subject a therapeutically effective amount of a peptide-antisense oligonucleotide conjugate, wherein the peptide-antisense oligonucleotide conjugate comprises an antisense oligonucleotide conjugated to a cell-penetrating peptide, and wherein the antisense oligonucleotide comprises all or a portion of PMO-002 (SEQ ID NO:2), MOE-012 (SEQ ID NO:12), or PMO-424 (SEQ ID NO:224).

In some embodiments, disclosed herein is a method of treating a subject having a neurodegenerative disease comprising administering to said subject a therapeutically effective amount of a peptide-antisense oligonucleotide conjugate, wherein the peptide-antisense oligonucleotide conjugate comprises an antisense oligonucleotide conjugated to a cell-penetrating peptide, and wherein the antisense oligonucleotide is selected from the group consisting of PMO-002 (SEQ ID NO:2), MOE-012 (SEQ ID NO:12), or PMO-424 (SEQ ID NO:224) In some embodiments, the subject is a human subject. In some embodiments, the neurodegenerative disease is Alzheimer's Disease.

In some embodiments, disclosed herein is a peptide-antisense oligonucleotide conjugate according to claim 1 for use in a method of inducing Exon-2 skipping in the CD33 gene during pre-mRNA splicing, comprising introducing into a cell the peptide-antisense oligonucleotide conjugate of claim 1, wherein the peptide-antisense oligonucleotide conjugate hybridizes to a target region of the CD33 gene and induces Exon-2 skipping during pre-mRNA splicing of the CD33 gene.

In some embodiments, disclosed herein is a peptide-antisense oligonucleotide conjugate for use in a method of inducing Exon-2 skipping in the CD33 gene during pre-mRNA splicing, comprising introducing into a cell a peptide-antisense oligonucleotide conjugate, wherein the peptide-antisense oligonucleotide conjugate hybridizes to a target region of the CD33 gene and induces Exon-2 skipping during pre-mRNA splicing of the CD33 gene. In some embodiments, the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length. In some embodiments, the antisense oligonucleotide comprises one or more non-natural sugar moieties, one or more non-natural internucleotide linkages, or one or more non-natural sugar moieties and one or more non-natural internucleotide linkages. In some embodiments, the antisense oligonucleotide comprises one or more modified sugar moieties. In some embodiments, the antisense oligonucleotide comprises a phosphorodiamidate morpholino oligomer (PMO). In some embodiments, the peptide-antisense oligonucleotide has a CD33 Exon-2 skipping efficiency of 30% or greater according to a Standard Exon-Skipping Efficiency Assay for PMO ASOs. In some embodiments, the antisense oligonucleotide comprises a methoxyethyl ribose oligomer (MOE). In some embodiments, the antisense oligonucleotide has a CD33 Exon-2 skipping efficiency of 30% or greater according to a Standard Exon-Skipping Efficiency Assay for MOE ASOs. In some embodiments, the antisense oligonucleotide comprises one or more non-natural internucleotide linkages. In some embodiments, all of the one or more non-natural internucleotide linkages have an Sp configuration. In some embodiments, all of the one or more non-natural internucleotide linkages have an Rp configuration In some embodiments, the one or more non-natural internucleotide linkages comprise one or more non-natural internucleotide linkages having an Sp configuration and one or more non-natural internucleotide linkages having an Rp configuration. In some embodiments, the antisense oligonucleotide comprises one or more modified nucleobases. In some embodiments, the antisense oligonucleotide further comprises a pharmaceutically acceptable carrier or excipient.

In some embodiments, disclosed herein is a peptide-antisense oligonucleotide conjugate for use in a method of inducing Exon-2 skipping in the CD33 gene during pre-mRNA splicing, comprising introducing into a cell a peptide-antisense oligonucleotide conjugate, wherein the peptide-antisense oligonucleotide conjugate hybridizes to a target region of the CD33 gene and induces Exon-2 skipping during pre-mRNA splicing of the CD33 gene.

In some embodiments, disclosed herein is a peptide-antisense oligonucleotide conjugate for use in a method of inducing Exon-2 skipping in the CD33 gene during pre-mRNA splicing, comprising introducing into a cell the peptide-antisense oligonucleotide conjugate, wherein the peptide-antisense oligonucleotide conjugate hybridizes to a target region of the CD33 gene and induces Exon-2 skipping during pre-mRNA splicing of the CD33 gene. In some embodiments, the cell is an animal cell. In some embodiments, the cell is a human cell.

In some embodiments, disclosed herein is a peptide-antisense oligonucleotide conjugate for use in a method of treating a subject having a neurodegenerative disease, comprising administering to said subject a therapeutically effective amount of a peptide-antisense oligonucleotide conjugate. In some embodiments, the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length. In some embodiments, the antisense oligonucleotide comprises one or more non-natural sugar moieties, one or more non-natural internucleotide linkages, or one or more non-natural sugar moieties and one or more non-natural internucleotide linkages. In some embodiments, the antisense oligonucleotide comprises one or more non-natural sugar moieties. In some embodiments, the antisense oligonucleotide comprises a phosphorodiamidate morpholino oligomer (PMO). In some embodiments, the peptide-antisense oligonucleotide conjugate has a CD33 Exon-2 skipping efficiency of 30% or greater according to a Standard Exon-Skipping Efficiency Assay for PMO ASOs. In some embodiments, the antisense oligonucleotide comprises a methoxyethyl ribose oligomer (MOE). In some embodiments, the antisense oligonucleotide has a CD33 Exon-2 skipping efficiency of 30% or greater according to a Standard Exon-Skipping Efficiency Assay for MOE ASOs. In some embodiments, the antisense oligonucleotide comprises one or more non-natural internucleotide linkages. In some embodiments, all of the one or more non-natural internucleotide linkages have an Sp configuration. In some embodiments, all of the one or more non-natural internucleotide linkages have an Rp configuration. In some embodiments, the one or more non-natural internucleotide linkages comprise one or more non-natural internucleotide linkages having an Sp configuration and one or more non-natural internucleotide linkages having an Rp configuration. In some embodiments, the antisense oligonucleotide comprises one or more modified nucleobases. In some embodiments, the peptide-antisense oligonucleotide further comprises a pharmaceutically acceptable carrier or excipient. In some embodiments, the neurodegenerative disease is Alzheimer's Disease.

In some embodiments, disclosed herein is a peptide comprising a cyclic peptide comprising a lipoic acid group. In some embodiments, the lipoic acid group is an (R)-lipoic acid group. In some embodiments, the lipoic acid group is an (S)-lipoic acid group. In some embodiments, the cyclic peptide comprises 4 to 40 amino acids, optionally wherein the cyclic peptide comprises 6 to 10 amino acids. In some embodiments, the cyclic peptide comprises 1 to 5 arginine residues, optionally wherein the cyclic peptide comprises 2 to 4 arginine residues. In some embodiments, the cyclic peptide comprises 1-5 aromatic hydrophobic amino acids, optionally wherein the cyclic peptide comprises 2-4 aromatic hydrophobic amino acids. In some embodiments, the cyclic peptide is chosen from:

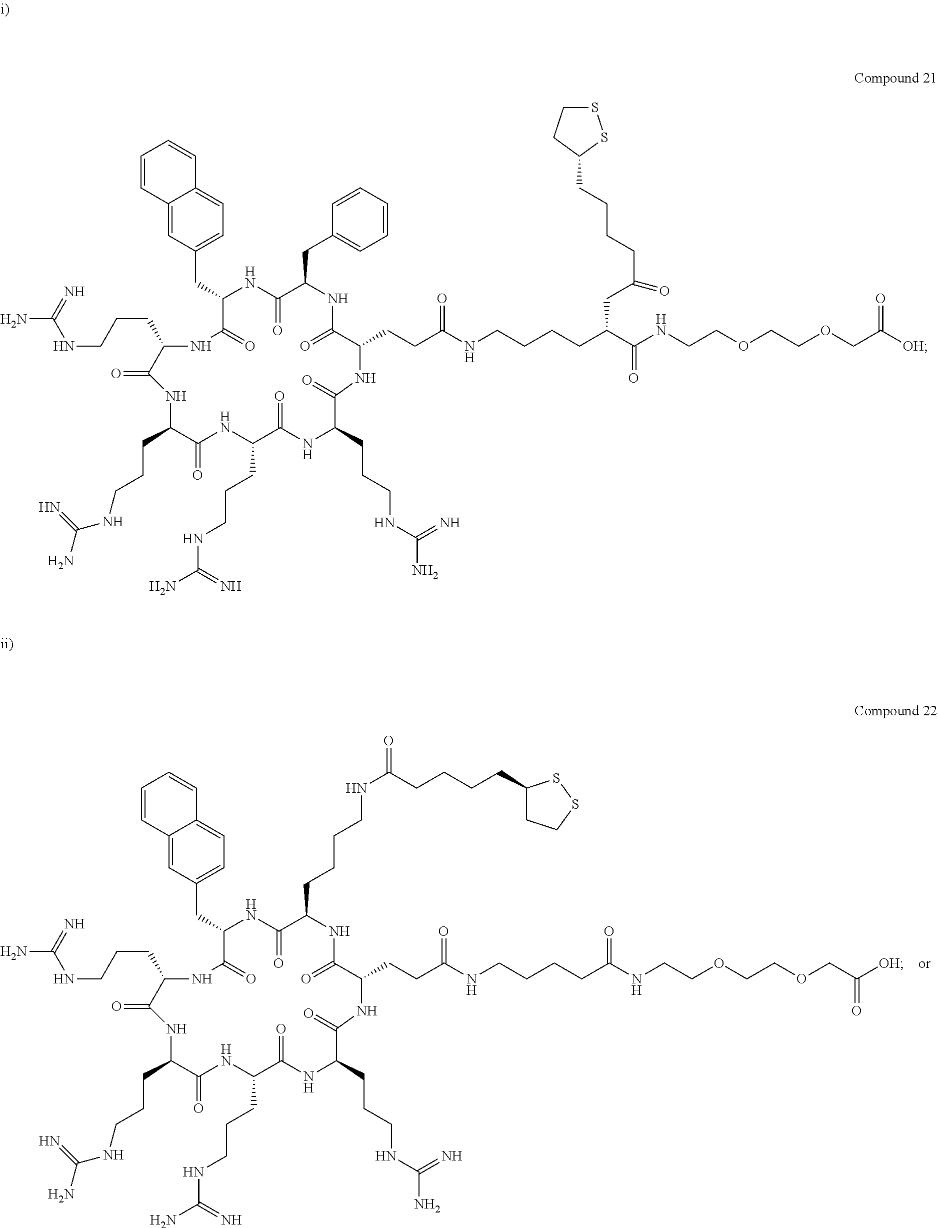 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCC[C@H](CC(=O)CCCC[C@@H]2CCSS2)C(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O ; N=C(N)NCCC[C@@H]1NC(=O)[C@H](Cc2ccc3ccccc3c2)NC(=O)[C@@H](CCCCNC(=O)CCCC[C@@H]2CCSS2)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

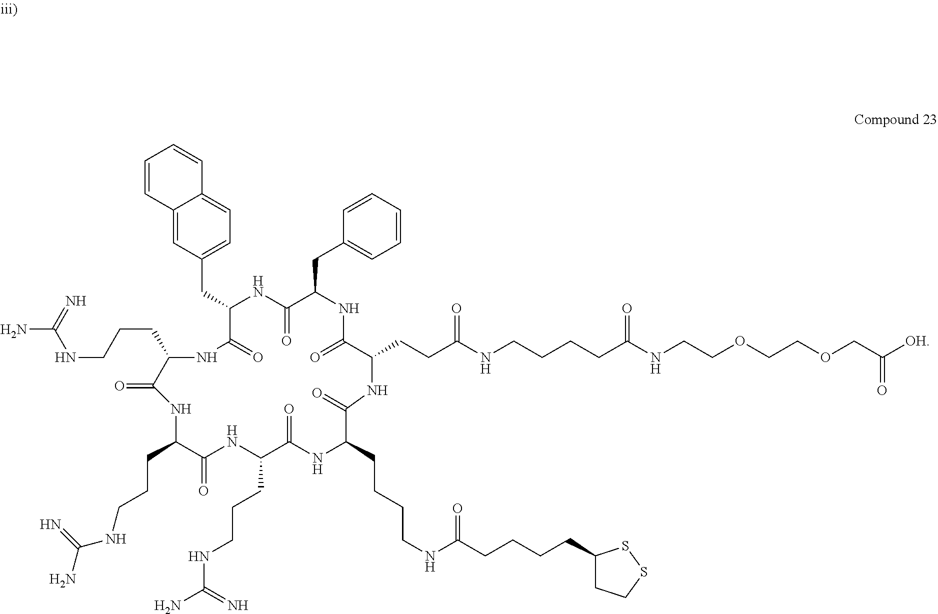 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCCNC(=O)CCCC[C@@H]2CCSS2)NC(=O)[C@H](CCCNC(=N)N)NC(=O)[C@@H](CCCNC(=N)N)NC1=O]

In some embodiments, the cyclic peptide comprises two or more lipoic acid groups. In some embodiments, the two or more lipoic acid groups comprise two or more (R)-lipoic acid groups, two or more (S)-lipoic acid groups, and/or one or more (R)-lipoic acid groups and one or more (S)-lipoic acid groups.

In some embodiments, disclosed herein is a method of making a peptide, wherein the peptide synthesized according to General Procedure C.

In some embodiments, disclosed herein is a peptide comprising a cyclic lactam group. In some embodiments, the peptide is a cell-penetrating peptide. In some embodiments, the peptide is a cyclic peptide. In some embodiments, the peptide comprises 4 to 40 amino acids, optionally wherein the peptide comprises 6 to 10 amino acids. In some embodiments, at least one amino acid of the peptide comprises the cyclic lactam group. In some embodiments, the cyclic lactam group is an eight, nine, or ten-membered ring. In some embodiments, the cyclic lactam group has a structure according to Formula III:

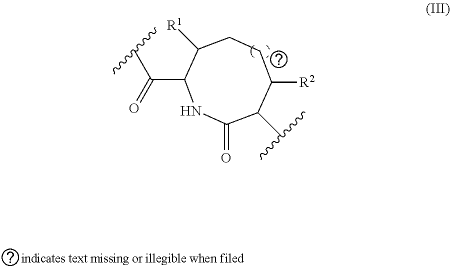

wherein: R¹ and R² are each independently selected from the group consisting of H, an aryl group, a heteroaryl group, an alkylaryl group, an arylalkyl group, a linear alkyl group, a branched alkyl group, and a guanidine-comprising group, wherein each of R¹ and R² is optionally substituted with one or more substituents; and n is an integer from 1 to 3. In some embodiments, the cyclic lactam comprises at least one side chain group. In some embodiments, the side chain group is R¹ or R². In some embodiments, R¹ and/or R² independently comprise a substituted or unsubstituted aryl group. the side chain group comprises a natural or non-natural group comprising aromatic group, a linear alkyl group, a branched alkyl group, a functionalized alkyl group, a guanidine group, a proline group, a lipophilic group, or an arginine group. In some embodiments, the aryl group is selected from the group consisting of a phenyl group, a benzyl group, and a naphthyl group. In some embodiments, R¹ and/or R² independently comprise a substituted or unsubstituted guanidine-comprising group. In some embodiments, the guanidine-comprising group is —(CH₂)₂CN₃H₄. In some embodiments, the peptide comprises 1-5 arginine residues, optionally wherein the peptide comprises 2-4 arginine residues.

In some embodiments, disclosed herein is a cyclic peptide comprising at least one amino acid having a structure according to Formula IV:

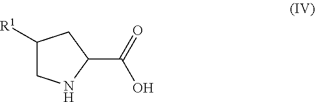

wherein R¹ comprises an aryl group or a guanidine-comprising group, wherein R¹ is optionally substituted with one or more substituents. In some embodiments, the aryl group is selected from the group consisting of a benzyl group, a phenyl group, and a naphthyl group. In some embodiments, the guanidine-comprising group is —(CH₂)₂—CN₃H₄.

In some embodiments, disclosed herein is a cyclic peptide comprising at least one oxadiazole linkage having a structure according to Formula V:

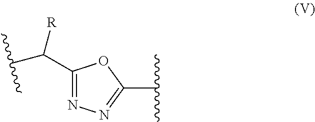

wherein R comprises a substituted or unsubstituted aryl group. In some embodiments, the aryl group is selected from the group consisting of a phenyl group, a benzyl group, a naphthyl group, and a methyl naphthyl group.

### BRIEF DESCRIPTION OF THE FIGURES

This application file contains figures in color. Copies of this patent application publication with color drawing(s) will be provided by the Office upon request and payment of the necessary fee.

FIG. 1 shows the levels of CD33 mRNA in plasma and cerebrospinal fluid in patients relative to the rs3865444 SNP. C=rs3865444-C, A=rs3865444-A.

FIG. 2 shows various cognitive results in patients with the rs3865444-A allele vs. patients with the rs201074739 indel frameshift allele.

FIG. 3 shows various physiological results in patients with the rs3865444-A allele vs. patients with the rs201074739 indel allele.

FIG. 4 shows the levels of CD33 mRNA in plasma and cerebrospinal fluid in patients relative to the rs201074739 indel.

FIG. 5 shows HPLC chromatogram and HRMS trace of PMO-424.

FIG. 6 shows HPLC chromatogram and HRMS trace of PMO-324.

FIG. 7 shows Tm of PMO-324, PMO-424, and PMO-224.

FIG. 8 shows HPLC chromatogram and HRMS trace of PMO-502.

FIG. 9 shows HPLC chromatogram and HRMS trace of PMO-402.

FIG. 10 shows Tm of PMO-402, PMO-502, and PMO-002.

FIG. 11 shows chromatogram of PMO-424 with N3′-trityl group (resin cleaved).

FIG. 12 shows the melting temperature of MOE-012, MOE-277, and MOE-278.

FIG. 13 shows the HPLC elution profile of stereopure ASOs MOE-288 to MOE-292 and stereorandom ASO MOE-252.

FIG. 14 shows in vitro Exon-2 skipping efficiencies for peptide-ASO conjugates at several doses using mouse bone-marrow derived macrophages.

FIG. 15 shows the in vivo activity of Compound 30 at a 30 μg dose and PMO-002 at 30 μg, 100 μg and 300 μg doses.

FIG. 16 shows the duration of in vivo skipping activity of Compound 30 with a single 30 μg ICV dose.

FIG. 17 shows the brain concentration of Compound 30 and naked PMO-002 after a single 30 μg ICV dose.

FIG. 18 shows the in vivo skipping activity in the cortex of Compound 31 at 3 μg, 10 μg, 30 μg and 60 μg and Sp-PMO-424 at 30 μg and 100 μg ICV dose.

FIG. 19 shows the in vivo skipping activity in the cortex and hippocampus of lipoic acid-containing peptides Compound 32, Compound 33, and Compound 34 at a 10 μg ICV dose, with 10 μg Compound 30 for comparison.

FIG. 20 shows examples of cyclic lactam amino acids.

FIG. 21 shows examples of cell penetrating peptides with lactam building blocks.

FIG. 22 shows an exemplary synthesis of unsaturated amino acids.

FIG. 23 shows an exemplary synthesis of 8-membered lactams with phenyl and 2-naphthalene side chains.

FIG. 24 shows an exemplary synthesis of lactams with guanidine side chains.

FIG. 25 shows an exemplary synthesis of lactams via consecutive Claisen rearrangements.

FIG. 26 shows an exemplary synthesis of 9- and 10-membered lactam amino acid rings.

FIG. 27 shows an exemplary synthesis of 9- and 10-membered lactam amino acid rings with guanidine side chains.

FIG. 28 shows examples of cell penetrating peptides with modified proline residues.

FIG. 29 shows examples of cell penetrating peptides containing 1,3,4-oxadiazole linkages.

FIG. 30 shows in vitro cellular uptake data for Compound 160, Compound 161, and Compound 162.

FIG. 31 shows the in vivo activity of Compound 166/167 and Compound 168/169 at a 30 μg dose.

FIG. 32 shows pharmacodynamic data for Compound 31 and Compound 33 in a 90-day duration study with transgenic hCD33 mice.

FIG. 33 shows pharmacokinetic data for Compound 31 and Compound 33 in a 90-day duration study with transgenic hCD33 mice.

FIG. 34 shows efficacy data for Compound 31 in 5×FAD hCD33 mice.

### DEFINITIONS

The term “oligonucleotide” is used herein to refer to a nucleotide sequence comprising at least ten DNA or RNA nucleotides.

The term “antisense oligonucleotide,” abbreviated as “ASO,” is used herein to refer to a nucleotide sequence comprising an antisense sequence that is sufficiently complementary to a target nucleotide sequence in order to form a stable double stranded hybrid with the target nucleotide sequence. In some embodiments, the target nucleotide sequence is an RNA nucleotide sequence. Unless otherwise specified, ASOs represented herein are displayed in the 5′ to 3′ orientation.

The term “peptide” is used herein to refer to a compound comprising two or more proteogenic or non-proteogenic amino acids.

The term “amino acid” is used herein to refer to a chemical compound containing an amine group and a carboxylic acid group in the same compound.

The term “proteogenic amino acid” is used herein to refer to an amino acid that is naturally included in a peptide.

The term “non-proteogenic amino acid” is used herein to refer to an amino acid that is not naturally included in a peptide. Non-proteogenic amino acids include naturally occurring amino acids or synthesized amino acids.

The term “cell penetrating peptide” or “CPP” is used herein to refer to a peptide that can penetrate a cell membrane, and can penetrate a cell membrane when conjugated to an antisense oligonucleotide.

The term “nucleobase” is used herein to refer to a base that is a component of a nucleoside. Example nucleobases include adenine, guanine, thymine, cytosine, and uracil.

The term “nucleoside” is used herein to refer to a nucleobase covalently linked to a sugar. Examples of naturally occurring and non-natural nucleosides are described below.

The term “nucleotide” is used herein to refer to a nucleoside covalently linked to a phosphate group. Examples of naturally occurring nucleotides include adenosine, thymidine, uridine, cytidine, 5-methylcytidine, and guanosine. Description and examples of non-natural nucleotides are described below.

The term “pharmaceutically acceptable salt” is used herein to refer to acid addition salts or base addition salts of the compounds in the present disclosure. A pharmaceutically acceptable salt is any salt which retains the activity of the parent compound and does not impart any unduly deleterious or undesirable effect on a subject to whom it is administered and in the context in which it is administered. Pharmaceutically acceptable salts include, but are not limited to, metal complexes and salts of both inorganic and carboxylic acids. Pharmaceutically acceptable salts also include metal salts such as sodium, calcium, potassium, magnesium, aluminum, iron, manganese, and complex salts. In addition, pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycolic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. A CPP-ASO that is a pharmaceutically acceptable salt may comprise a pharmaceutically acceptable salt of a CPP of the CPP-ASO, a pharmaceutically acceptable salt of an ASO of the CPP-ASO, and/or a pharmaceutically acceptable salt of any linker that may conjugate a CPP to an ASO of the CPP-ASO.

Within the ASO structure of the CPP-ASO, the phosphate groups are commonly referred to as forming the “internucleotide linkages” of the ASO. The naturally occurring internucleotide linkage of RNA and DNA is a 3′ to 5′ phosphodiester linkage. A “phosphoramidate” group comprises phosphorus having three attached oxygen atoms and one attached nitrogen atom, while a “phosphorodiamidate” group comprises phosphorus having two attached oxygen atoms and two attached nitrogen atoms. A “phosphorotriamidate” group (or a phosphoric acid triamide group) comprises phosphorus having one attached oxygen atom and three attached nitrogen atoms. In the uncharged or the cationic internucleotide linkages of the morpholino-based ASOs described herein, one nitrogen is always pendant to the linkage chain. The second nitrogen, in a phosphorodiamidate linkage, is typically the ring nitrogen in a morpholino ring structure.

The term “non-natural” is used herein to refer to molecules that contain man-made modifications relative to their naturally occurring counterparts. In some embodiments, “non-natural” may refer to one or more nucleotide subunits having at least one modification selected from (i) a modified internucleotide linkage, e.g., an internucleotide linkage other than the standard phosphodiester linkage found in naturally-occurring oligonucleotides, (ii) modified sugar moieties, e.g., moieties other than ribose or deoxyribose moieties found in naturally occurring oligonucleotides, (iii) modified nucleobases, e.g., bases other than those found in naturally occurring oligonucleotides, or (iv) any combination of the foregoing. In some embodiments, the ASO of a CPP-ASO is chosen from ASOs that do not have a phosphorus atom in the internucleotide linkage (backbone). In some embodiments, the ASO has a phosphorodiamidate or phosphorothioate modified internucleotide linkage (backbone).

The term “morpholino” is used herein to refer to a nucleotide that contains a morpholinyl ring instead of a ribose.

The term “morpholino-based ASO” is used herein to refer to an ASO with at least one nucleotide containing a morpholinyl ring instead of a ribose.

The term “stereo-controlled” is used herein to describe when a nucleotide and/or an oligonucleotide is designed or selected to have a particular stereochemistry. In some embodiments, the nucleobase portion of a nucleotide or oligonucleotide, including any and all non-natural modifications, is stereo-controlled. In some embodiments, the nucleoside portion of a nucleotide or oligonucleotide, including any and all non-natural modifications, is stereo-controlled. In some embodiments, the internucleotide linkage portion of a nucleotide or oligonucleotide, including any and all non-natural modifications, is stereo-controlled. In some embodiments, a nucleotide may comprise one or a combination of these stereo-controlled portions. In some embodiments, an oligonucleotide may comprise a combination of nucleotides that comprise a combination of stereo-controlled nucleotides. In some embodiments, an oligonucleotide may comprise a combination of nucleotides that are stereo-controlled and not stereo-controlled. In some embodiments, the proportion of stereo-controlled nucleotides ranges from 10%-100%, such as 15%-100%, 20%-100%, 30%-100%, 40%-100%, 50%-100%, 50%-90%, 50%-95%, 60%-100%, 60%-90%, 60%-95%, 70%-100%, 70%-90%, 70%-95%, 80-100%, 80%-90%, 80%-95%, 90-100%, 90%-95%, 90%-96%, 90%-97%, 90%-98%, 90%-99%, 95%-98%, 95%-99%, 95-100%, 50%-90%, or 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%, of nucleotides.

When applied to nucleotides, the term “stereopure” is used herein to describe when at least 90% of nucleotides in an oligonucleotide are stereo-controlled. In some embodiments, the proportion of stereo-controlled nucleotides in a stereopure CPP-ASO ranges from 90-100%, 95-100%, 90%-95%, 90%-96%, 90%-97%, 90%-98%, 90%-99%, 95%-98%, 95%-99%, or 90%, 91%, 92%, 93%, 94%, 95%, 96%, 97%, 98%, 99%, or 100%, or at least 90%, 91%, 92%, 93%, 94%, 95%, 96%, 97%, 98%, or 99%, of nucleotides. In some embodiments, all or a portion of nucleotides within an oligonucleotide are stereo-controlled so that they are stereopure in the same way, i.e., all or a portion of the nucleotides are stereo-controlled, and they are designed or selected to have the same stereochemistry. In some embodiments, all or a portion of nucleotides within an oligonucleotide are stereo-controlled so that they are not stereopure in the same way, i.e., all or a portion of the nucleotides are stereo-controlled, but they are designed or selected to have different stereochemistry. When applied to the internucleotide linkage portion of an oligonucleotide, the term “stereopure” is used to describe when at least 90% of the internucleotide linkages are stereo-controlled. In some embodiments, the proportion of stereo-controlled internucleotide linkages in a stereopure CPP-ASO ranges from 90-100%, 95-100%, 90%-95%, 90%-96%, 90%-97%, 90%-98%, 90%-99%, 95%-98%, 95%-99%, or 90%, 91%, 92%, 93%, 94%, 95%, 96%, 97%, 98%, 99%, or 100%, or at least 90%, 91%, 92%, 93%, 94%, 95%, 96%, 97%, 98%, or 99%, of internucleotide linkages. In some embodiments, all or a portion of internucleotide linkages within an oligonucleotide are stereo-controlled so that they are stereopure in the same way, i.e., all or a portion of the internucleotide linkages are stereo-controlled, and they are designed or selected to have the same stereochemistry. In some embodiments, all or a portion of internucleotide linkages within an oligonucleotide are stereo-controlled so that they are not stereopure in the same way, i.e., all or a portion of the internucleotide linkages are stereo-controlled, but they are designed or selected to have different stereochemistry. In some embodiments, the internucleotide linkages are phosphorodiamidate linkages. In some embodiments, the internucleotide linkages are phosphorothioate linkages.

Stereochemistry for (Rp, Sp) and phosphate (PO) internucleotide linkages is illustrated as the following:

Sp [symbol] Rp [symbol] PO

[symbol]

Stereochemistry for Rp, Sp, and PO internucleotide linkages is also illustrated as follows: S=Sp, R=Rp, O=phosphate.

For example, the stereochemistry of the internucleotide linkages of MOE-298 can be shown using either of the following illustrations:

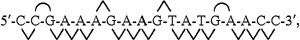

### or (5′-CCGAAAGAAGTATGAACC-3′) (SEQ ID NO:252); Stereopattern: SOSSSRSSSRSSSOSSS.

When applied to nucleotides, the term “stereorandom” is used herein to describe when the nucleotides in an oligonucleotide are not stereo-controlled. When applied to internucleotide linkages, the term “stereorandom” is used herein to describe when the internucleotide linkages in an oligonucleotide are not stereo-controlled. In some embodiments, the internucleotide linkages are phosphorodiamidate linkages. In some embodiments, the internucleotide linkages are phosphorothioate linkages.

The term “complementary” is used herein to describe when the corresponding positions of at least two nucleotide sequences are occupied by nucleotides which can hydrogen bond with each other.

The term “hybridize” is used herein to describe the binding of two complementary nucleotide sequences, forming one double stranded molecule. When a sufficient number of corresponding nucleotides in two sequences can hydrogen bond with each other, i.e., they are sufficiently complementary, they may form a stable hybrid. It is understood in the art that 100% complementarity is not necessary for a CPP-ASO to hybridize with a target sequence.

The term “sufficient complementarity” is used herein to indicate a level of complementarity sufficient to permit an ASO of a CPP-ASO to bind to its target sequence and form a stable hybrid. In some embodiments, the complementarity of the ASO and the target sequence is at least 99%, or 98%, or 97%, or 96%, or 95%, or 94%, or 93%, or 92%, or 91%, or 90%, or 89%, or 88%, or 87%, or 86%, or 85%, or 84%, or 83%, or 82%, or 81%, or 80%, or 79%, or 78%, or 77%, or 76%, or 75%, or 74%, or 73%, or 72%, or 71%, or 70%.

The term “sequence similarity” is used herein to express the similarity of two CPP-ASOs. Sequence similarity is expressed as a percentage of nucleotides shared between two CPP-ASOs. It is understood that identical sequences have 100% sequence similarity.

The terms “target region” and “target sequence” are used interchangeably herein to designate a nucleotide sequence to which an ASO of a CPP-ASO will hybridize under physiological conditions. It is not necessary for the ASO and the target region to be 100% complementary, so long as there is sufficient complementarity for the ASO to hybridize to the target sequence and form a stable hybrid. The ASO may hybridize to all or a portion of the target sequence.

The terms “treat,” “treating,” or “treatment” are used herein to refer to ameliorating a disease or disorder (i.e., slowing or arresting or reducing the development of the disease or at least one of the clinical symptoms thereof). The terms also refer to alleviating or ameliorating at least one physical parameter including those which may not be discernible by the patient. The terms also refer to modulating the disease or disorder, either physically (e.g., through stabilization of a discernible symptom), physiologically, (e.g., through stabilization of a physical parameter), or both.

The terms “prevent,” “preventing,” or “prevention” are used herein to refer to inhibiting or delaying the onset of a disease or disorder.

The term “therapeutically effective amount” is used herein to refer to the amount of a therapeutic agent or composition effective in prevention or treatment of a disorder or disease. In some embodiments, this includes an amount of a therapeutic agent or composition effective in the prevention or treatment of a neurodegenerative disease.

The term “pharmaceutically acceptable” is used herein to refer to a molecular entity or composition that is pharmaceutically useful and not biologically or otherwise undesirable.

The term “carrier” is used herein to refer to a diluent, adjuvant, excipient, or vehicle with which the compound is administered.

The term “excipient” as used herein refers to any ingredient in a pharmaceutical composition other than the active ingredient.

As used herein, “skipping efficiency” of an oligonucleotide is calculated using the following formula:

$$Skipping \% = \frac{( Skipped Value )}{( Skipped ) + ( Un - skipped )} \times 100$$

and is represented on a scale of 0 to 100, wherein 100 represents 100% skipping of CD33 Exon-2. “Skipping efficiency” of an oligonucleotide as used herein is experimentally determined using one of three Standard Exon-Skipping Efficiency Assays depending on the type of antisense oligonucleotide. For antisense oligonucleotides comprising phosphorodiamidate morpholino oligomers, the Standard Exon-Skipping Efficiency Assay for PMO CPP-ASOs defined below is used; for antisense oligonucleotides comprising methoxyethyl ribose oligomers, the Standard Exon-Skipping Efficiency Assay for MOE CPP-ASOs defined below is used; and for antisense oligonucleotides that do not comprise phosphorodiamidate morpholino or methoxyethyl ribose oligomers, the Standard Exon-Skipping Efficiency Assay for non-PMOs and non-MOEs described below is used.

The Standard Exon-Skipping Efficiency Assay for CPP-ASOs includes using mouse bone-marrow derived macrophages (mBMDM) cells that were cultured and maintained using appropriate media suggested in the vendor protocols (Dulbecco's Modified Eagle's Medium containing 10% fetal bovine serum). The Assay is performed in 96 well plate format, seeding about 50,000 cells per well and treating with the PMO CPP-ASO at a concentration of 0.5 μM without additional transfection reagents. Cells are incubated at 37 °C in a cell culture incubator for 48 hours before isolating the total RNA. Total RNA is isolated and converted to cDNA per vendor protocol, then Taqman gene expression assays are used to quantify Exon-2 skipped CD33 (Forward primer CGCTGCTGCTACTGCTG (SEQ ID NO:207); Reverse Primer: TTCTAGAGTGCCAGGGATGA (SEQ ID NO:208); and probe: TGTGGGCAGACTTGACCCACAG (SEQ ID NO:209)) and un-skipped CD33 (Forward primer: GGATGGAGAGAGGAAGTA (SEQ ID NO:210) or TTCGGATGGAGAGAGGAAGTA (SEQ ID NO:291); Reverse Primer GTGCCAGGGATGAGGATTT (SEQ ID NO:211); and probe: TGCATGTGACAGACTTGACCCACA (SEQ ID NO:212)) mRNA transcripts. Mouse house-keeping gene HPRT1 (Assay ID: Hs02800695_m1; ThermoFisher Scientific) expression is used to normalize the target transcript expressions.

The term “alkyl” is used herein to refer to saturated hydrocarbons having one or more carbon atoms, including straight-chain alkyl groups (e.g., methyl, ethyl, propyl, butyl, pentyl, hexyl, etc.), branched-chain alkyl groups (e.g., isopropyl, tert-butyl, sec-butyl, isobutyl, etc.), cyclic alkyl groups (also referred to as “cycloalkyl” groups), and alkyl-substituted alkyl groups (e.g., alkyl-substituted cycloalkyl groups and cycloalkyl-substituted alkyl groups). The terms “alkenyl” and “alkynyl” refer to unsaturated aliphatic groups that are analogous to alkyls but contain at least one double or triple carbon-carbon bond, respectively.

The term “alkoxy” is used herein to refer to an alkyl group linked to the remainder of the molecule through an oxygen atom. Examples of alkoxy groups include methoxy, ethoxy, isopropyloxy, propoxy, butoxy, and pentoxy groups. The alkoxy groups can be straight-chain or branched.

The term “alkoxyalkyl” is used herein to refer to an alkyl group substituted with an alkoxy group.

The term “arylalkyl” is used herein to refer to an alkyl group substituted with an aryl group (e.g., phenylmethyl (i.e., benzyl)).

The term “alkylaryl” is used herein to refer to an aryl group substituted with an alkyl group (e.g., p-methylphenyl (i.e., p-tolyl)).

The terms “carbocycle” and “carbocyclic” are used herein to a closed ring hydrocarbon structure (e.g., monocyclic, polycyclic ring structure). Carbocyclic groups may be saturated or unsaturated. Additionally, carbocyclic groups may be aromatic or non-aromatic.

The terms “heterocycle” and “heterocyclic” are used herein to refer to a closed ring structure (e.g., monocyclic, polycyclic ring structure) in which one or more of the atoms in the closed ring structure is a heteroatom (i.e., an atom other than carbon). Certain exemplary heteroatoms include nitrogen, oxygen, and sulfur. Heterocyclic groups may be saturated or unsaturated. Additionally, heterocyclic groups may be aromatic or non-aromatic.

Unless otherwise defined, all other scientific and technical terms have the same meaning as commonly understood to one of ordinary skill in the art. Such scientific and technical terms are explained in the literature, for example: J. Sambrook, E. F. Fritsch, and T Maniatis, Molecular Cloning: A Laboratory Manual, Second Edition, Books 1-3, Cold Spring Harbor Laboratory Press (1989); Martin, Remington's Pharmaceutical Sciences, 18th Edition, Mack Publishing Co (1990); Glover, DNA Cloning: A Practical Approach, Volumes I and II, MRL Press, Ltd. (1985); and Ausubel, F et al., Current Protocols in Molecular Biology, Greene Publishing Associates/Wiley Intersciences (2002).

### DETAILED DESCRIPTION

Disclosed herein are novel CPP-ASOs. In some embodiments, the CPP-ASOs are directed to a target sequence in the CD33 pre-mRNA. In some embodiments, the CPP-ASOs are complementary to all or a portion of a target sequence in the CD33 pre-mRNA, represented in SEQ ID NO:1 (5′-GGGCAGGTGA GTGGCTGTGG GGAGAGGGGT TGTCGGGCTG GGCCGAGCTG ACCCTCGTTT CCCCACAGGG GCCCTGGCTA TGGATCCAAA TTTCTGGCTG CAAGTGCAGG AGTCAGTGAC GGTACAGGAG GGTTTGTGCG TCCTCGTGCC CTGCACTTTC TTCCATCCCA TACCCTACTA CGACAAGAAC TCCCCAGTTC ATGGTTACTG GTTCCGGGAA GGAGCCATTA TATCCAGGGA CTCTCCAGTG GCCACAAACA AGCTAGATCA AGAAGTACAG GAGGAGACTC AGGGCAGATT CCGCCTCCTT GGGGATCCCA GTAGGAACAA CTGCTCCCTG AGCATCGTAG ACGCCAGGAG GAGGGATAAT GGTTCATACT TCTTTCGGAT GGAGAGAGGA AGTACCAAAT ACAGTTACAA ATCTCCCCAG CTCTCTGTGC ATGTGACAGG TGAGGCACAG GCTTCAGAAG TGGCCGCAAG GGAAGTTCAT GGGTACTGCA GGGCAGGGCT GGGATGGGAC CCTGGTACTG-3′). SEQ ID NO:1 includes Exon-2 and portions of the bordering introns of the CD33 gene. This target sequence is involved in Exon-2 skipping, which also occurs when CD33 mRNA includes the rs3865444-A SNP. When this Exon-2 skipping occurs, pre-mRNA containing the SNP is spliced so that Exon-2 is not included in the final transcript.

In some embodiments, the CPP-ASO has a CD33 Exon-2 skipping efficiency of at least 25%, at least 30%, at least 35%, at least 40%, at least 50%, at least 60%, at least 70%, at least 80%, at least 90%, or at least 99% according to a Standard Exon-Skipping Efficiency Assay for the CPP-ASO. In some embodiments, the CPP-ASO has a CD33 Exon-2 skipping efficiency in a range of 25% to 99%, 30% to 99%, 35% to 99%, 40% to 99%, 50% to 99%, 60% to 99%, 70% to 99%, 80% to 99%, or 90% to 99% according to a Standard Exon-Skipping Efficiency Assay for the CPP-ASO. In some embodiments, the CPP-ASO has a CD33 Exon-2 skipping efficiency of 25%, 30%, 35%, 40%, 45%, 50%, 55%, 60%, 65%, 70%, 75%, 80%, 85%, 90%, 95%, or 99% according to a Standard Exon-Skipping Efficiency Assay for the CPP-ASO.

In some embodiments, the ASO of a CPP-ASO is 16-30 nucleotides long. In some embodiments, the ASO of a CPP-ASO is 20-30 nucleotides long. In some embodiments, the ASO of a CPP-ASO is 25-30 nucleotides long. In some embodiments, the ASO of a CPP-ASO is 21-30 nucleotides long. In some embodiments, the ASO of a CPP-ASO is 21-25 nucleotides long. In some embodiments, the ASO of a CPP-ASO is 18-21 nucleotides long. In some embodiments, the ASO of a CPP-ASO is 18-25 nucleotides long. In some embodiments, the ASO of a CPP-ASO is 16, 17, 18, 19, 20, 21, 22, 23, 24, 25, 26, 27, 28, 29, or 30 nucleotides long.

In some embodiments, the ASO of a CPP-ASO comprises 16-30, such as 18-30, nucleotides. In some embodiments, the ASO of a CPP-ASO consists of 16-30, such as 18-30, nucleotides.

Also disclosed herein are novel CPP-ASOs complementary to all or a portion of a 10- to 16-nucleotide target sequence in the CD33 pre-mRNA, represented in SEQ ID NO:1, which includes Exon-2 and portions of the bordering introns of the CD33 gene. In some embodiments, the ASO of the CPP-ASOs are 10-14 nucleotides long. In some embodiments, the ASO of the CPP-ASOs are 10, 11, 12, 13, 14, 15, or 16 nucleotides long.

In some embodiments, the CPP-ASOs are complementary to all or a portion of a 16- to 30-nucleotide target sequence in the CD33 pre-RNA, are sufficiently complementary to the target sequence to form a stable hybrid, and are 16-30 nucleotides in length. In some embodiments, the ASO portions of the CPP-ASOs are sufficiently complementary to all or a portion of a 25-nucleotide target sequence in the CD33 pre-RNA.

In some embodiments, the ASO of the CPP-ASOs have one of the specific sequences disclosed in Table 1 or 2.

TABLE 1

| | | SEQ. |
|---|---|---|
| | | ID |
| ASO NO: | Sequence (5′ to 3′) | NO: |
| PMO-016 | CAGTACCAGGGTCCCATCCCAGCCC | 16 |
| PMO-017 | CCAGGGTCCCATCCCAGCCCTGCCC | 17 |
| PMO-018 | GTCCCATCCCAGCCCTGCCCTGCAG | 18 |
| PMO-019 | ATCCCAGCCCTGCCCTGCAGTACCC | 19 |
| PMO-020 | AGCCCTGCCCTGCAGTACCCATGAA | 20 |
| PMO-021 | TGCCCTGCAGTACCCATGAACTTCC | 21 |
| PMO-022 | TGCAGTACCCATGAACTTCCCTTGC | 22 |
| PMO-023 | TACCCATGAACTTCCCTTGCGGCCA | 23 |
| PMO-024 | ATGAACTTCCCTTGCGGCCACTTCT | 24 |
| PMO-025 | CTTCCCTTGCGGCCACTTCTGAAGC | 25 |
| PMO-026 | CTTGCGGCCACTTCTGAAGCCTGTG | 26 |
| PMO-027 | GGCCACTTCTGAAGCCTGTGCCTCA | 27 |
| PMO-028 | CTTCTGAAGCCTGTGCCTCACCTGT | 28 |
| PMO-029 | GAAGCCTGTGCCTCACCTGTCACAT | 29 |
| PMO-030 | CTGTGCCTCACCTGTCACATGCACA | 30 |
| PMO-002 | CCTCACCTGTCACATGCACAGAGAG | 2 |
| PMO-003 | CCTGTCACATGCACAGAGAGCTGGG | 3 |
| PMO-031 | CACATGCACAGAGAGCTGGGGAGAT | 31 |
| PMO-032 | GCACAGAGAGCTGGGGAGATTTGTA | 32 |
| PMO-033 | GAGAGCTGGGGAGATTTGTAACTGT | 33 |
| PMO-034 | CTGGGGAGATTTGTAACTGTATTTG | 34 |
| PMO-035 | GAGATTTGTAACTGTATTTGGTACT | 35 |
| PMO-036 | TTGTAACTGTATTTGGTACTTCCTC | 36 |
| PMO-037 | ACTGTATTTGGTACTTCCTCTCTCC | 37 |
| PMO-004 | ATTTGGTACTTCCTCTCTCCATCCG | 4 |
| PMO-038 | GTACTTCCTCTCTCCATCCGAAAGA | 38 |
| PMO-039 | TCCTCTCTCCATCCGAAAGAAGTAT | 39 |
| PMO-005 | TCTCCATCCGAAAGAAGTATGAACC | 5 |
| PMO-040 | ATCCGAAAGAAGTATGAACCATTAT | 40 |
| PMO-041 | AAAGAAGTATGAACCATTATCCCTC | 41 |
| PMO-042 | AGTATGAACCATTATCCCTCCTCCT | 42 |
| PMO-043 | GAACCATTATCCCTCCTCCTGGCGT | 43 |
| PMO-044 | ATTATCCCTCCTCCTGGCGTCTACG | 44 |
| PMO-045 | CCCTCCTCCTGGCGTCTACGATGCT | 45 |
| PMO-046 | CTCCTGGCGTCTACGATGCTCAGGG | 46 |
| PMO-047 | GGCGTCTACGATGCTCAGGGAGCAG | 47 |
| PMO-048 | CTACGATGCTCAGGGAGCAGTTGTT | 48 |
| PMO-049 | ATGCTCAGGGAGCAGTTGTTCCTAC | 49 |
| PMO-050 | CAGGGAGCAGTTGTTCCTACTGGGA | 50 |
| PMO-051 | AGCAGTTGTTCCTACTGGGATCCCC | 51 |
| PMO-052 | TTGTTCCTACTGGGATCCCCAAGGA | 52 |
| PMO-053 | CCTACTGGGATCCCCAAGGAGGCGG | 53 |
| PMO-054 | TGGGATCCCCAAGGAGGCGGAATCT | 54 |
| PMO-055 | TCCCCAAGGAGGCGGAATCTGCCCT | 55 |
| PMO-056 | AAGGAGGCGGAATCTGCCCTGAGTC | 56 |
| PMO-057 | GGCGGAATCTGCCCTGAGTCTCCTC | 57 |
| PMO-058 | AATCTGCCCTGAGTCTCCTCCTGTA | 58 |
| PMO-059 | GCCCTGAGTCTCCTCCTGTACTTCT | 59 |
| PMO-060 | GAGTCTCCTCCTGTACTTCTTGATC | 60 |
| PMO-061 | TCCTCCTGTACTTCTTGATCTAGCT | 61 |
| PMO-062 | CTGTACTTCTTGATCTAGCTTGTTT | 62 |
| PMO-063 | CTTCTTGATCTAGCTTGTTTGTGGC | 63 |
| PMO-064 | TGATCTAGCTTGTTTGTGGCCACTG | 64 |
| PMO-065 | TAGCTTGTTTGTGGCCACTGGAGAG | 65 |
| PMO-066 | TGTTTGTGGCCACTGGAGAGTCCCT | 66 |
| PMO-067 | GTGGCCACTGGAGAGTCCCTGGATA | 67 |
| PMO-068 | CACTGGAGAGTCCCTGGATATAATG | 68 |
| PMO-069 | GAGAGTCCCTGGATATAATGGCTCC | 69 |
| PMO-070 | TCCCTGGATATAATGGCTCCTTCCC | 70 |
| PMO-071 | GGATATAATGGCTCCTTCCCGGAAC | 71 |
| PMO-072 | TAATGGCTCCTTCCCGGAACCAGTA | 72 |
| PMO-073 | GCTCCTTCCCGGAACCAGTAACCAT | 73 |
| PMO-074 | TTCCCGGAACCAGTAACCATGAACT | 74 |
| PMO-075 | GGAACCAGTAACCATGAACTGGGGA | 75 |
| PMO-076 | CAGTAACCATGAACTGGGGAGTTCT | 76 |
| PMO-077 | ACCATGAACTGGGGAGTTCTTGTCG | 77 |
| PMO-078 | GAACTGGGGAGTTCTTGTCGTAGTA | 78 |
| PMO-079 | GGGGAGTTCTTGTCGTAGTAGGGTA | 79 |
| PMO-080 | GTTCTTGTCGTAGTAGGGTATGGGA | 80 |
| PMO-081 | TGTCGTAGTAGGGTATGGGATGGAA | 81 |
| PMO-082 | TAGTAGGGTATGGGATGGAAGAAAG | 82 |
| PMO-083 | GGGTATGGGATGGAAGAAAGTGCAG | 83 |
| PMO-006 | TGGGATGGAAGAAAGTGCAGGGCAC | 6 |
| PMO-084 | TGGAAGAAAGTGCAGGGCACGAGGA | 84 |
| PMO-085 | GAAAGTGCAGGGCACGAGGACGCAC | 85 |
| PMO-086 | TGCAGGGCACGAGGACGCACAAACC | 86 |
| PMO-087 | GGCACGAGGACGCACAAACCCTCCT | 87 |
| PMO-088 | GAGGACGCACAAACCCTCCTGTACC | 88 |
| PMO-089 | CGCACAAACCCTCCTGTACCGTCAC | 89 |
| PMO-090 | AAACCCTCCTGTACCGTCACTGACT | 90 |
| PMO-091 | CTCCTGTACCGTCACTGACTCCTGC | 91 |
| PMO-092 | GTACCGTCACTGACTCCTGCACTTG | 92 |
| PMO-093 | GTCACTGACTCCTGCACTTGCAGCC | 93 |
| PMO-094 | TGACTCCTGCACTTGCAGCCAGAAA | 94 |
| PMO-095 | CCTGCACTTGCAGCCAGAAATTTGG | 95 |
| PMO-096 | ACTTGCAGCCAGAAATTTGGATCCA | 96 |
| PMO-007 | CAGCCAGAAATTTGGATCCATAGCC | 7 |
| PMO-097 | AGAAATTTGGATCCATAGCCAGGGC | 97 |
| PMO-098 | TTTGGATCCATAGCCAGGGCCCCTG | 98 |
| PMO-099 | ATCCATAGCCAGGGCCCCTGTGGGG | 99 |
| PMO-100 | TAGCCAGGGCCCCTGTGGGGAAACG | 100 |
| PMO-101 | AGGGCCCCTGTGGGGAAACGAGGGT | 101 |
| PMO-008 | CCCTGTGGGGAAACGAGGGTCAGCT | 8 |
| PMO-102 | TGGGGAAACGAGGGTCAGCTCGGCC | 102 |
| PMO-103 | AAACGAGGGTCAGCTCGGCCCAGCC | 103 |
| PMO-104 | AGGGTCAGCTCGGCCCAGCCCGACA | 104 |
| PMO-105 | CAGCTCGGCCCAGCCCGACAACCCC | 105 |
| PMO-106 | CGGCCCAGCCCGACAACCCCTCTCC | 106 |
| PMO-107 | CAGCCCGACAACCCCTCTCCCCACA | 107 |
| PMO-108 | CGACAACCCCTCTCCCCACAGCCAC | 108 |
| PMO-109 | ACCCCTCTCCCCACAGCCACTCACC | 109 |
| PMO-110 | TCTCCCCACAGCCACTCACCTGCCC | 110 |

TABLE 2

| | | SEQ. |
|---|---|---|
| | | ID |
| ASO NO: | Sequence (5′ to 3′) | NO: |
| MOE-111 | CAGTACCAGGGTCCCATCCC | 111 |
| MOE-112 | CCAGGGTCCCATCCCAGCCC | 112 |
| MOE-113 | GTCCCATCCCAGCCCTGCCC | 113 |
| MOE-114 | ATCCCAGCCCTGCCCTGCAG | 114 |
| MOE-115 | AGCCCTGCCCTGCAGTACCC | 115 |
| MOE-116 | TGCCCTGCAGTACCCATGAA | 116 |
| MOE-117 | TGCAGTACCCATGAACTTCC | 117 |
| MOE-118 | TACCCATGAACTTCCCTTGC | 118 |
| MOE-119 | ATGAACTTCCCTTGCGGCCA | 119 |
| MOE-120 | CTTCCCTTGCGGCCACTTCT | 120 |
| MOE-121 | CTTGCGGCCACTTCTGAAGC | 121 |
| MOE-122 | GGCCACTTCTGAAGCCTGTG | 122 |
| MOE-123 | CTTCTGAAGCCTGTGCCTCA | 123 |
| MOE-124 | GAAGCCTGTGCCTCACCTGT | 124 |
| MOE-125 | CTGTGCCTCACCTGTCACAT | 125 |
| MOE-126 | CCTCACCTGTCACATGCACA | 126 |
| MOE-127 | CCTGTCACATGCACAGAGAG | 127 |
| MOE-009 | CACATGCACAGAGAGCTGGG | 9 |
| MOE-128 | GCACAGAGAGCTGGGGAGAT | 128 |
| MOE-010 | GAGAGCTGGGGAGATTTGTA | 10 |
| MOE-129 | CTGGGGAGATTTGTAACTGT | 129 |
| MOE-130 | GAGATTTGTAACTGTATTTG | 130 |
| MOE-131 | TTGTAACTGTATTTGGTACT | 131 |
| MOE-132 | ACTGTATTTGGTACTTCCTC | 132 |
| MOE-133 | ATTTGGTACTTCCTCTCTCC | 133 |
| MOE-134 | GTACTTCCTCTCTCCATCCG | 134 |
| MOE-135 | TCCTCTCTCCATCCGAAAGA | 135 |
| MOE-011 | TCTCCATCCGAAAGAAGTAT | 11 |
| MOE-012 | ATCCGAAAGAAGTATGAACC | 12 |
| MOE-136 | AAAGAAGTATGAACCATTAT | 136 |
| MOE-137 | AGTATGAACCATTATCCCTC | 137 |
| MOE-138 | GAACCATTATCCCTCCTCCT | 138 |
| MOE-139 | ATTATCCCTCCTCCTGGCGT | 139 |
| MOE-140 | CCCTCCTCCTGGCGTCTACG | 140 |
| MOE-141 | CTCCTGGCGTCTACGATGCT | 141 |
| MOE-142 | GGCGTCTACGATGCTCAGGG | 142 |
| MOE-143 | CTACGATGCTCAGGGAGCAG | 143 |
| MOE-013 | ATGCTCAGGGAGCAGTTGTT | 13 |
| MOE-144 | CAGGGAGCAGTTGTTCCTAC | 144 |
| MOE-145 | AGCAGTTGTTCCTACTGGGA | 145 |
| MOE-146 | TTGTTCCTACTGGGATCCCC | 146 |
| MOE-147 | CCTACTGGGATCCCCAAGGA | 147 |
| MOE-148 | TGGGATCCCCAAGGAGGCGG | 148 |
| MOE-149 | TCCCCAAGGAGGCGGAATCT | 149 |
| MOE-150 | AAGGAGGCGGAATCTGCCCT | 150 |
| MOE-151 | GGCGGAATCTGCCCTGAGTC | 151 |
| MOE-152 | AATCTGCCCTGAGTCTCCTC | 152 |
| MOE-153 | GCCCTGAGTCTCCTCCTGTA | 153 |
| MOE-014 | GAGTCTCCTCCTGTACTTCT | 14 |
| MOE-154 | TCCTCCTGTACTTCTTGATC | 154 |
| MOE-155 | CTGTACTTCTTGATCTAGCT | 155 |
| MOE-156 | CTTCTTGATCTAGCTTGTTT | 156 |
| MOE-157 | TGATCTAGCTTGTTTGTGGC | 157 |
| MOE-158 | TAGCTTGTTTGTGGCCACTG | 158 |
| MOE-159 | TGTTTGTGGCCACTGGAGAG | 159 |
| MOE-160 | GTGGCCACTGGAGAGTCCCT | 160 |
| MOE-161 | CACTGGAGAGTCCCTGGATA | 161 |
| MOE-162 | GAGAGTCCCTGGATATAATG | 162 |
| MOE-162 | GAGAGTCCCTGGATATAATG | 162 |
| MOE-163 | TCCCTGGATATAATGGCTCC | 163 |
| MOE-164 | GGATATAATGGCTCCTTCCC | 164 |
| MOE-165 | TAATGGCTCCTTCCCGGAAC | 165 |
| MOE-165 | TAATGGCTCCTTCCCGGAAC | 165 |
| MOE-166 | GCTCCTTCCCGGAACCAGTA | 166 |
| MOE-167 | TTCCCGGAACCAGTAACCAT | 167 |
| MOE-168 | GGAACCAGTAACCATGAACT | 168 |
| MOE-169 | CAGTAACCATGAACTGGGGA | 169 |
| MOE-170 | ACCATGAACTGGGGAGTTCT | 170 |
| MOE-171 | GAACTGGGGAGTTCTTGTCG | 171 |
| MOE-172 | GGGGAGTTCTTGTCGTAGTA | 172 |
| MOE-173 | GTTCTTGTCGTAGTAGGGTA | 173 |
| MOE-174 | TGTCGTAGTAGGGTATGGGA | 174 |
| MOE-175 | TAGTAGGGTATGGGATGGAA | 175 |
| MOE-176 | GGGTATGGGATGGAAGAAAG | 176 |
| MOE-177 | TGGGATGGAAGAAAGTGCAG | 177 |
| MOE-178 | TGGAAGAAAGTGCAGGGCAC | 178 |
| MOE-179 | GAAAGTGCAGGGCACGAGGA | 179 |
| MOE-180 | TGCAGGGCACGAGGACGCAC | 180 |
| MOE-181 | GGCACGAGGACGCACAAACC | 181 |
| MOE-182 | GAGGACGCACAAACCCTCCT | 182 |
| MOE-015 | CGCACAAACCCTCCTGTACC | 15 |
| MOE-183 | AAACCCTCCTGTACCGTCAC | 183 |
| MOE-184 | CTCCTGTACCGTCACTGACT | 184 |
| MOE-185 | GTACCGTCACTGACTCCTGC | 185 |
| MOE-186 | GTCACTGACTCCTGCACTTG | 186 |
| MOE-187 | TGACTCCTGCACTTGCAGCC | 187 |
| MOE-188 | CCTGCACTTGCAGCCAGAAA | 188 |
| MOE-189 | ACTTGCAGCCAGAAATTTGG | 189 |
| MOE-190 | CAGCCAGAAATTTGGATCCA | 190 |
| MOE-191 | AGAAATTTGGATCCATAGCC | 191 |
| MOE-192 | TTTGGATCCATAGCCAGGGC | 192 |
| MOE-193 | ATCCATAGCCAGGGCCCCTG | 193 |
| MOE-194 | TAGCCAGGGCCCCTGTGGGG | 194 |
| MOE-195 | AGGGCCCCTGTGGGGAAACG | 195 |
| MOE-196 | CCCTGTGGGGAAACGAGGGT | 196 |
| MOE-197 | TGGGGAAACGAGGGTCAGCT | 197 |
| MOE-198 | AAACGAGGGTCAGCTCGGCC | 198 |
| MOE-199 | AGGGTCAGCTCGGCCCAGCC | 199 |
| MOE-200 | CAGCTCGGCCCAGCCCGACA | 200 |
| MOE-201 | CGGCCCAGCCCGACAACCCC | 201 |
| MOE-202 | CAGCCCGACAACCCCTCTCC | 202 |
| MOE-203 | CGACAACCCCTCTCCCCACA | 203 |
| MOE-204 | ACCCCTCTCCCCACAGCCAC | 204 |
| MOE-205 | TCTCCCCACAGCCACTCACC | 205 |
| MOE-206 | CCACAGCCACTCACCTGCCC | 206 |

In some embodiments, PMO and MOE ASOs were designed to cover CD33 Exon-2 and its surrounding introns (SEQ ID NO: 1) in 20-25 nucleotide sections that moved down SEQ ID NO:1 5′ to 3′ five nucleotides at a time. Regions that exhibited increased Exon-2 skipping activity were identified where two or more consecutive PMO or MOE ASOs that are complementary to a section of SEQ ID NO:1 showed increased Exon-2 skipping activity. Those regions are identified below and in Table 3:
Region 1: (SEQ ID NO:213) (see, e.g., PMO-002 and PMO-003)
Region 2: (SEQ ID NO:214) (see, e.g., PMO-036, PMO-037, PMO-004, PMO-038, PMO-039, and PMO-005)
Region 3: (SEQ ID NO:215) (see, e.g., PMO-082, PMO-083, and PMO-006)
Region 4: (SEQ ID NO:216) (see, e.g., PMO-096, PMO-007, and PMO-097)
Region 5: (SEQ ID NO:217) (see, e.g., MOE-009, MOE-128, and MOE-010)
Region 6: (SEQ ID NO:218) (see, e.g., MOE-135, MOE-011, and MOE-012)
Region 7: (SEQ ID NO:219) (see, e.g., MOE-015, MOE-183, and MOE-184)
Region 8: (SEQ ID NO:220) (see, e.g., MOE-196 and MOE-197).

TABLE 3

| | | SEQ. |
|---|---|---|
| Region | | ID |
| NO: | Sequence (5′ to 3′) | NO: |
| Region 1 | 5′-TCTCCCCAGCTCTCTGTGCATGTGACAGG | 213 |
| | TGAGGCACA-3′ | |
| Region 2 | 5′ - | 214 |
| | TAATGGTTCATACTTCTTTCGGATGGAGAGAG | |
| | GAAGTACCAAATACAGTTACAAATCT-3′ | |
| Region 3 | 5′ - | 215 |
| | CCTCGTGCCCTGCACTTTCTTCCATCCCATAC | |
| | CCTACTACGAC-3′ | |
| Region 4 | 5′ - | 216 |
| | AGGGGCCCTGGCTATGGATCCAAATTTCTGGC | |
| | TGCAAGTGCAG-3′ | |
| Region 5 | 5′ - | 217 |
| | ACAGTTACAAATCTCCCCAGCTCTCTGTGCAT | |
| | GTGACAGGTGAGG-3′ | |
| Region 6 | 5′ -GGTTCATACTTCTTTCGGATGGAGAGAG | 218 |
| | GAAGTACCAAAT-3′ | |
| Region 7 | 5′ -GCAGGAGTCAGTGACGGTACAGGAGGGT | 219 |
| | TTGTGCG-3′ | |
| Region 8 | 5′ -GGCCGAGCTGACCCTCGTTTCCCCACAG | 220 |
| | GGGCCC-3′ | |

In some embodiments, the ASO of a CPP-ASO is complementary to a region of SEQ ID NO:1 showing increased Exon-2 skipping activity, including but not limited to Regions 1, and 2, 3, 4, 5, 6, 7, and 8. In some embodiments, the ASO of a CPP-ASO is complementary to at least a portion of SEQ ID NO:213; SEQ ID NO:214; SEQ ID NO:215; SEQ ID NO:216; SEQ ID NO:217; SEQ ID NO:218; SEQ ID NO:219; and/or SEQ ID NO:220.

In some embodiments, the ASO of a CPP-ASO is complementary to at least a portion of Region 1 of SEQ ID NO:1.

In some embodiments, the ASO of the CPP-ASO has a sequence disclosed in Table 4:

TABLE 4

| | | SEQ. |
|---|---|---|
| | | ID |
| ASO NO: | Sequence (5′ to 3′) | NO: |
| PMO-221 | 5′ -CCTCACCTGTCACATGCACAGAG-3′ | 221 |
| PMO-222 | 5′ -TCACCTGTCACATGCACAGAGAG-3′ | 222 |
| PMO-223 | 5′ -CTCACCTGTCACATGCACAGAGA-3′ | 223 |
| PMO-224 | 5′ -CCTCACCTGTCACATGCACAG-3′ | 224 |
| PMO-225 | 5′ -ACCTGTCACATGCACAGAGAG-3′ | 225 |
| PMO-226 | 5′ -TCACCTGTCACATGCACAGAG-3′ | 226 |
| PMO-227 | 5′ -TCACCTGTCACATGCACAGAGAGCT-3′ | 227 |
| PMO-228 | 5′ -CCTGTGCCTCACCTGTCACATGCAC-3′ | 228 |
| PMO-229 | 5′ -GTGCCTCACCTGTCACATGCACAGA-3′ | 229 |
| PMO-230 | 5′ -TGCCTCACCTGTCACATGCACAGAG-3′ | 230 |
| PMO-231 | 5′ -CTCACCTGTCACATGCACAGAGAGC-3′ | 231 |
| PMO-232 | 5′ -CACCTGTCACATGCACAGAGAGCTG-3′ | 232 |
| PMO-233 | 5′ -ACCTGTCACATGCACAGAGAGCTGG-3′ | 233 |
| PMO-234 | 5′ -CTGTCACATGCACAGAGAGCTGGGG-3′ | 234 |
| PMO-235 | 5′ -CCTGTCACATGCACAGAGAGCTG-3′ | 235 |
| PMO-236 | 5′ -TGTCACATGCACAGAGAGCTGGG-3′ | 236 |
| PMO-237 | 5′ -CTGTCACATGCACAGAGAGCTGG-3′ | 237 |
| PMO-238 | 5′ -TGTCACATGCACAGAGAGCTGG-3′ | 238 |
| PMO-239 | 5′ -TCACATGCACAGAGAGCTGGG-3′ | 239 |
| PMO-240 | 5′ -TGTCACATGCACAGAGAGCTG-3′ | 240 |

In some embodiments, the ASO of a CPP-ASO is complementary to at least a portion of Region 2 of SEQ ID NO: 1.

In some embodiments, the ASO of the CPP-ASO has a sequence disclosed in Table 5:

TABLE 5

| | | SEQ. |
|---|---|---|
| | | ID |
| ASO NO: | Sequence (5′ to 3′) | NO: |
| PMO-241 | 5′ -CTGTATTTGGTACTTCCTCTCTCCA-3′ | 241 |
| PMO-242 | 5′ -TGTATTTGGTACTTCCTCTCTCCAT-3′ | 242 |
| PMO-243 | 5′ -GTATTTGGTACTTCCTCTCTCCATC-3′ | 243 |
| PMO-244 | 5′ -TATTTGGTACTTCCTCTCTCCATCC-3′ | 244 |

In some embodiments, the ASO of a CPP-ASO is complementary to at least a portion of Region 6 (SEQ ID NO:218) of SEQ ID NO:1.

In some embodiments, the ASO of the CPP-ASO has a sequence disclosed in Table 6:

TABLE 6

| | | SEQ. |
|---|---|---|
| ASO NO: | Sequence (5′ to 3′) | ID NO: |
| MOE-245 | CTCCATCCGAAAGAAGTATG | 245 |
| MOE-246 | TCCATCCGAAAGAAGTATGA | 246 |
| MOE-247 | CCATCCGAAAGAAGTATGAA | 247 |
| MOE-248 | CATCCGAAAGAAGTATGAAC | 248 |
| MOE-249 | TCCGAAAGAAGTATGAACCA | 249 |
| MOE-250 | CCGAAAGAAGTATGAACCAT | 250 |
| MOE-251 | ATCCGAAAGAAGTATGAA | 251 |
| MOE-252 | CCGAAAGAAGTATGAACC | 252 |
| MOE-253 | TCCGAAAGAAGTATGAAC | 253 |
| MOE-254 | CCATCCGAAAGAAGTATG | 254 |
| MOE-255 | TCCATCCGAAAGAAGTAT | 255 |
| MOE-256 | GAAAGAAGTATGAACCAT | 256 |

In some embodiments, the CPP-ASOs may share sequence similarity with one of the CPP-ASOs disclosed in Tables 1, 2, and 4 to 6. In some embodiments, the CPP-ASO shares at least 99%, or 98%, or 97%, or 96%, or 95%, or 94%, or 93%, or 92%, or 91%, or 90%, or 89%, or 88%, or 87%, or 86%, or 85%, or 84%, or 83%, or 82%, or 81%, or 80%, or 79%, or 78%, or 77%, or 76%, or 75%, or 74%, or 73%, or 72%, or 71%, or 70% sequence similarity with one of the CPP-ASOs disclosed in Tables 1, 2, and 4 to 6.

In some embodiments, one or more nucleobases of the ASO of the CPP-ASOs comprise uracil. In some embodiments, one or more nucleobases of the ASO of a CPP-ASO comprise thymine. In some embodiments, one or more nucleosides of the ASO of a CPP-ASO comprise a ribose sugar moiety. In some embodiments, one or more nucleosides of the ASO of a CPP-ASO comprise a deoxyribose sugar moiety.

In some embodiments, the ASO of the CPP-ASOs comprise at least one chemically modified nucleotide. In some embodiments, the at least one chemical modification of the nucleotide is chosen from chemical modification of at least one nucleobase, chemical modification of at least one sugar moiety, chemical modification of at least one phosphate, and any combination of these modifications. In some embodiments, the at least one chemical modification improves the ability of the nucleotide to resist nuclease degradation.

Certain exemplary chemical modifications useful in this disclosure include chemical modifications of an ASO's phosphate backbone and non-natural internucleoside linkage(s). In some embodiments, the ASO of a CPP-ASO is chosen from ASOs having a chemically modified phosphate backbone. In some embodiments, the chemically modified phosphate backbone comprises one or more nitrogen atoms and/or one or more sulfur atoms. In some embodiments, for example, one or more non-bridging oxygen atoms in the phosphate backbone (e.g., in phosphodiester linkages) may be replaced by nitrogen or sulfur atoms. In some embodiments, the ASO of a CPP-ASO has a phosphoramidate, phosphorodiamidate, phosphorodithioate, or phosphorothioate modified backbone. In some embodiments, the ASO of a CPP-ASO is chosen from ASOs that do not have a phosphorus atom in the backbone. In some embodiments, the modified backbone is stereo-controlled.

Certain exemplary chemical modifications useful in this disclosure include chemical modifications of at least one sugar moiety in an ASO. In some embodiments, the ASO of a CPP-ASO comprises at least one chemically modified (e.g., a non-natural) sugar moiety.

In some embodiments, the ASO of a CPP-ASO comprises at least one chemically modified (e.g., a non-natural) sugar moiety that is chosen from sugar moieties substituted in at least one position. In some embodiments, the at least one chemically modified (e.g., a non-natural) sugar moiety is substituted in at least one position on the sugar chosen from the 2′, 3′ and 5′ positions. In some embodiments, the at least one substituent on the ASO's at least one substituted sugar moiety is chosen from hydroxyl; fluoro; alkoxy; amino; and substituted or unsubstituted, linear or branched C₁-C₁₀ alkyl groups, substituted or unsubstituted, linear or branched C₂-C₁₀ alkenyl groups, substituted or unsubstituted, linear or branched C₂-C₁₀ alkynyl groups, substituted or unsubstituted, linear or branched C₇-C₁₇ alkylaryl groups, substituted or unsubstituted, linear or branched C₃-C₁₀ allyl groups, substituted or unsubstituted, linear or branched C₇-C₁₇ arylalkyl groups, and substituted or unsubstituted, linear or branched C₂-C₁₀ alkoxyalkyl groups, each of which groups may optionally further comprise at least one heteroatom. In some embodiments, at least one substituent on at least one substituted sugar moiety of an ASO of a CPP-ASO comprises methoxy, methoxyethyl, ethoxy, propoxy, aminopropoxy, methoxyethoxy, dimethylaminoethoxy, or dimethylaminoethoxyethoxy. In some embodiments, one or more sugar moieties of the ASO of a CPP-ASO are chosen from pyranoses, derivatives of pyranoses, deoxypyranoses, derivatives of deoxypyranoses, riboses, derivatives of riboses, deoxyriboses, and derivatives of deoxyriboses. In some embodiments, the sugar moiety is stereo-controlled.

In some embodiments, the ASO of a CPP-ASO comprises at least one sugar moiety that is modified in a manner that creates a bicyclic sugar moiety. In some embodiments, the bicyclic sugar moiety is formed from a bridge modification between the 4′ and 2′ furanose ring atoms. In some embodiments, the bridge modification comprises at least one group that forms a bridge between the 4′ and 2′ furanose ring atoms. In some embodiments, at least one nucleotide in a given ASO of a CPP-ASO has a bridge modification. In some embodiments, at least one nucleotide in an ASO of a CPP-ASO is a locked nucleic acid (LNA).

In some embodiments, the ASO of a CPP-ASO comprises at least one sugar moiety comprising fewer than 5 ring atoms, such as 4 ring atoms. In some embodiments, the ASO of a CPP-ASO comprises at least one sugar moiety comprising more than 5 ring atoms, such as 6 ring atoms. In some embodiments, the ASO of a CPP-ASO comprises at least one sugar moiety comprising a morpholinyl ring. In some embodiments, the ASO of a CPP-ASO is a morpholino-based ASO. A morpholino-based ASO refers to an ASO comprising morpholino subunits, where morpholinyl rings replace ribose moieties. Certain exemplary internucleotide linkages for such morpholino-based ASOs include, for example, phosphoramidate or phosphorodiamidate internucleotide linkages joining the morpholinyl ring nitrogen of one morpholino subunit to the 4′ exocyclic carbon of an adjacent morpholino subunit. Each morpholino subunit comprises a purine or pyrimidine nucleobase, which may bind by base-specific hydrogen bonding to a nucleobase in a target sequence. In some embodiments, the morpholino-based ASO may include at least one further modification.

In some embodiments, the ASO of a CPP-ASO is a phosphorodiamidate morpholino oligomer (PMO). In some embodiments, the ASO of a CPP-ASO has the structure of Formula I:

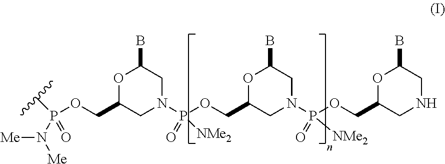

Where B is any nucleobase described herein and n is an integer in a range from 1 to 19, 1 to 23, 1 to 28, 1 to 30, 8 to 19, 8 to 23, 8 to 28, 8 to 30, 14 to 19, 14 to 23, 14 to 28, 14 to 30, 16 to 19, 16 to 23, 16 to 28, or 16 to 30.

In some embodiments, the ASO of a CPP-ASO is a methoxyethyl ribose oligomer (MOE). In some embodiments, the ASO of a CPP-ASO has the structure of Formula II:

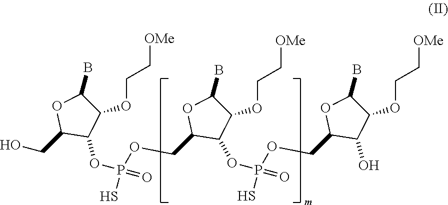

where B is any nucleobase described herein and m is an integer in a range from 1 to 19, 1 to 23, 1 to 28, 1 to 30, 8 to 19, 8 to 23, 8 to 28, 8 to 30, 14 to 19, 14 to 23, 14 to 28, 14 to 30, 16 to 19, 16 to 23, 16 to 28, or 16 to 30.

In some embodiments, both the sugar moiety and the linkage between the nucleobase and the sugar moiety of at least one nucleotide unit in the ASO of a CPP-ASO are replaced with non-natural groups. In some embodiments, the nucleobase units are maintained for hybridization with an appropriate nucleic acid target compound. In some embodiments, the ASO of a CPP-ASO is chosen from peptide nucleic acids (PNAs). In some embodiments, the sugar-backbone of at least one oligonucleotide in the PNA is replaced with an amide-containing backbone, for example, an aminoethylglycine backbone. In some embodiments, the nucleobases are retained and are bound directly or indirectly to aza nitrogen atoms of the amide portion of the backbone.

In some embodiments, the ASO of a CPP-ASO comprises at least one non-natural nucleobase (often referred to as “base”) (e.g., a nucleobase comprising one or more modifications or substitutions). Examples of non-natural nucleobases include 5-substituted pyrimidines (e.g., 5-methyl cytosine, 5-propynyl cytosine, 5-propynyl uracil), 6-azapyrimidines, and N-2, N-6, and O-6 substituted purines, including but not limited to 2-aminopropyladenine. Certain non-natural nucleobases are particularly useful for increasing the binding affinity of the oligomeric compounds of the disclosure. For example, 5-methylcytosine substitutions have been shown to increase nucleic acid duplex stability by 0.6-1.2 °C. In some embodiments, the modified nucleobase is stereo-controlled.

It is not necessary for all positions in a given ASO to be uniformly modified, and in fact, more than one of the aforementioned modifications may be incorporated in a single nucleotide within an ASO. In some embodiments, an ASO of a CPP-ASO may comprise one or more chemically modified nucleotides and one or more chemically unmodified nucleotides. ASOs may contain at least one region wherein the nucleotides are modified to confer upon them increased resistance to nuclease degradation, increased cellular uptake, and/or an additional region for increased binding affinity for the target nucleic acid.

Certain exemplary ASOs comprising a plurality of modifications to nucleobases, sugar moieties, and/or internucleotide linkages include ASOs having a sequence shown in Table 7. In Table 7, each nucleotide has a 2′-MOE ribose sugar moiety, each C represents a 5-methyl cytosine, each lower case letter represents a locked nucleic acid, each (-) represents a phosphodiester (PO) bond, each fX represents a 2′-fluoro ribonucleotide, and each mX represents a 2′-OMe ribonucleotide.

TABLE 7

| | | SEQ. |
|---|---|---|
| ASO NO: | Sequence (5′ to 3′) | ID NO: |
| MOE-257 | ATC-CGAAAGAAGTATGA-ACC | 12 |
| MOE-258 | ATCC-GAAAGAAGTATG-AACC | 12 |
| MOE-259 | ATCCG-AAAGAAGTAT-GAACC | 12 |
| MOE-260 | ATCCG-AAAGAAGTA-TGAACC | 12 |
| MOE-261 | ATCC-GAAAGA-AGTATG-AACC | 12 |
| MOE-262 | ATCC-gAAAGAAGTATGAACC | 12 |
| MOE-263 | ATCC-gAAAGAAGTATG-aACC | 12 |
| MOE-264 | ATCC-gAAAGAaGTATG-aACC | 12 |
| MOE-265 | CCGA-aAGAAGTATGAACC | 252 |
| MOE-266 | CCGA-aAGAAGTATG-aACC | 252 |
| MOE-267 | CCGA-aAGAAGtATG-aACC | 252 |
| MOE-268 | CCG-AAAGAAGTATGA-ACC | 252 |
| MOE-269 | CCGA-AAGAAGTATG-AACC | 252 |
| MOE-270 | CCGAA-AGAA-GTATG-AACC | 252 |
| MOE-271 | CCGAA-AGAAGTAT-GAACC | 252 |
| MOE-272 | CCG-A-AAGAAGTATGAACC | 252 |
| MOE-273 | CCG-AA-AGAAGTATGAACC | 252 |
| MOE-274 | CCGAAAGAAGTATG-A-ACC | 252 |
| MOE-275 | mAmTfCfCfGfAfAfAfGfAfAf | 12 |
| | GfTfAfTfGfAfAmCmC | |
| MOE-276 | fAfTfCfCfGmAmAmAmGmAmAm | 12 |
| | GmTmAfTfGfAfAfCfC | |

Due to potential three-dimensional variation of the sugar moieties, nucleobases, and internucleotide linkages, some nucleotides may share the same molecular formula but have a different spatial arrangement, i.e., some nucleotides may be stereoisomers. In some embodiments, for example, modification of a phosphodiester internucleotide linkage by replacing one or more oxygens with one or more nitrogen and/or sulfur atoms may cause the phosphorus atom of the linkage to become a chiral center. In some embodiments, the ASO of a CPP-ASO comprises one or more P-chiral internucleotide linkages in an Sp or Rp configuration. As an illustrative, non-limiting example, Sp and Rp configurations of an exemplary phosphorothioate linkage are shown below:

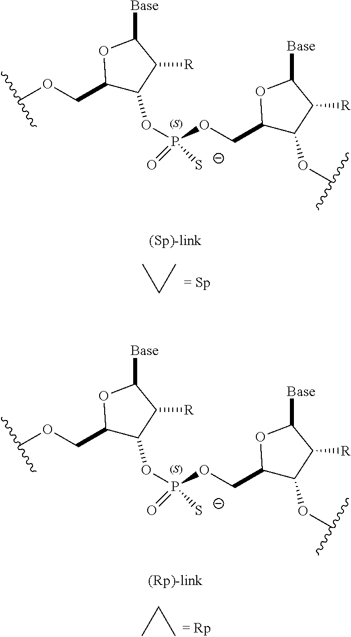

In some embodiments, the stereochemistries of nucleotides within a given ASO of a CPP-ASO are not controlled so as to make the ASO stereorandom. In some embodiments, the nucleotides within a given ASO of a CPP-ASO are stereo-controlled. In some embodiments, one or more nucleotides within a given ASO are stereo-controlled so as to make the ASO of a CPP-ASO stereopure. In some embodiments a given ASO of a CPP-ASO is a combination of stereo-controlled and stereorandom nucleotides.

In some embodiments, the proportion of stereo-controlled nucleotides in the ASO of a CPP-ASO is in a range from 10%-100%, 15%-100%, 20%-100%, 30%-100%, 40%-100%, 50%-100%, 50%-90%, 50%-95%, 60%-100%, 60%-90%, 60%-95%, 70%-100%, 70%-90%, 70%-95%, 80-100%, 80%-90%, 80%-95%, 90-100%, 90%-95%, 90%-96%, 90%-97%, 90%-98%, 90%-99%, 95%-98%, 95%-99%, 95-100%, 50%-90%, or 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%.

In some embodiments, the proportion of Sp internucleotide linkages in the ASO of a CPP-ASO is at least 80%, 85%, 90%, 95%, 98%, or 99%. In some embodiments, the proportion of Sp internucleotide linkages in the ASO of a CPP-ASO is 80%, 85%, 90%, 91%, 92%, 93%, 94%, 95%, 96%, 97%, 98%, 99%, or 100%.

In some embodiments, the proportion of Rp internucleotide linkages in the ASO of a CPP-ASO is at least 80%, 85%, 90%, 95%, 98%, or 99%. In some embodiments, the proportion of Rp internucleotide linkages in the ASO of a CPP-ASO is 80%, 85%, 90%, 91%, 92%, 93%, 94%, 95%, 96%, 97%, 98%, 99%, or 100%.

In some embodiments, the ASO of a CPP-ASO is a stereopure PMO ASO having a sequence disclosed in Table 8:

TABLE 8

| | | SEQ. |
|---|---|---|
| | | ID |
| ASO NO: | Sequence (5′ to 3′) | NO: |
| PMO-324 | 5′ -CCTCACCTGTCACATGCACAG-3′ | 224 |
| | (all Rp internucleotide linkages) | |
| PMO-424 | 5′-CCTCACCTGTCACATGCACAG-3′ | 224 |
| | (all Sp internucleotide linkages) | |
| PMO-402 | 5′-CCTCACCTGTCACATGCACAGAGAG-3′ | 2 |
| | (all Rp internucleotide linkages) | |
| PMO-502 | 5′-CCTCACCTGTCACATGCACAGAGAG-3′ | 2 |
| | (all Sp internucleotide linkages) | |

In some embodiments, the ASO of a CPP-ASO is a stereopure MOE ASO having a sequence disclosed in Table 9:

TABLE 9

| ASO# | Sequence (5′ to 3′) | SEQ ID No: |
|---|---|---|
| MOE-277 | 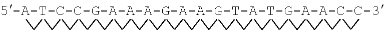 | 12 |
| MOE-278 | 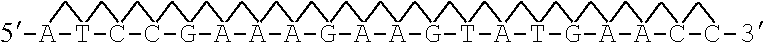 | 12 |
| MOE-279 | 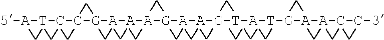 | 12 |
| MOE-280 | 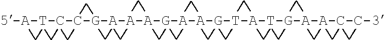 | 12 |
| MOE-281 | 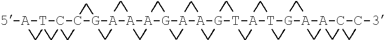 | 12 |
| MOE-282 | 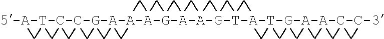 | 12 |
| MOE-283 | 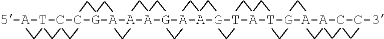 | 12 |
| MOE-284 | 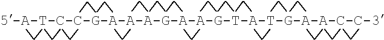 | 12 |
| MOE-285 | 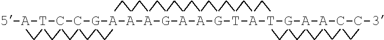 | 12 |
| MOE-286 | 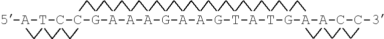 | 12 |
| MOE-287 | 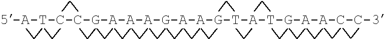 | 12 |
| MOE-288 | 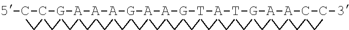 | 252 |
| MOE-289 | 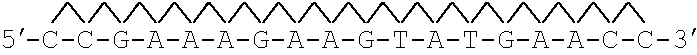 | 252 |
| MOE-290 | 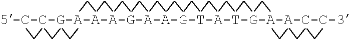 | 252 |
| MOE-291 | 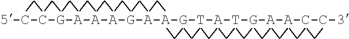 | 252 |
| MOE-292 | 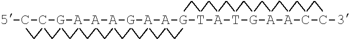 | 252 |
| MOE-293 | 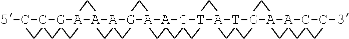 | 252 |
| MOE-294 | 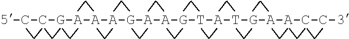 | 252 |
| MOE-295 | 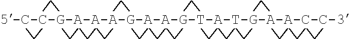 | 252 |
| MOE-296 | 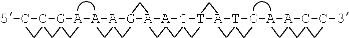 | 252 |
| MOE-297 | 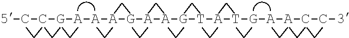 | 252 |
| MOE-298 | 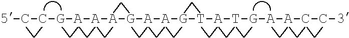 | 252 |
| MOE-299 | 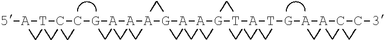 | 12 |
| MOE-300 | 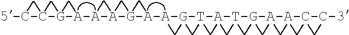 | 252 |
| MOE-301 | 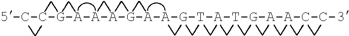 | 252 |
| MOE-303 | 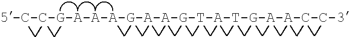 | 252 |
| MOE-304 | 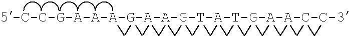 | 252 |
| MOE-305 | 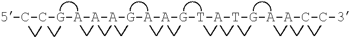 | 252 |
| MOE-306 | 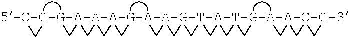 | 252 |
| MOE-307 | 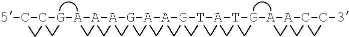 | 252 |
| MOE-308 | 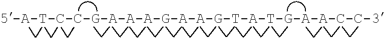 | 12 |
| MOE-309 | 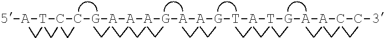 | 12 |
| MOE-310 | 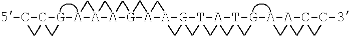 | 252 |
| MOE-311 | 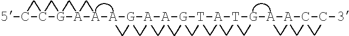 | 252 |

In some ASOs of CPP-ASOs, it is possible for some modifications to the sugar moieties, nucleobases, internucleotide linkages and/or stereo-controlled nucleotides to be arranged in regions that create a particular motif for the ASO. In some embodiments, the ASO of a CPP-ASO comprises at least two regions. In some embodiments, the ASO of a CPP-ASO comprises three regions: one region near the 5′ end of the ASO, one region near the 3Tend of the ASO, and a gap region between the two other regions. This type of arrangement is known as a gapmer motif. The length of each motif can be equal to other motifs within the ASO of a CPP-ASO, or the length of each motif can be independent of the length of other motifs within the ASO. In some embodiments, one or more sugar moieties in an ASO of a CPP-ASO are modified so that a block of sugar moieties in one region of the ASO is different from a block of sugar moieties in a different region of the ASO. In some embodiments, an ASO of a CPP-ASO comprises modified sugar moieties arranged in a gapmer motif. In some embodiments, one or more nucleobases in an ASO of a CPP-ASO are modified so that a block of nucleobases in one region of the ASO is different from a block of nucleobases in a different region of the ASO. In some embodiments, an ASO of a CPP-ASO comprises modified nucleobases arranged in a gapmer motif. In some embodiments, one or more internucleotide linkages in an ASO of a CPP-ASO are modified so that a block of internucleotide linkages in one region of the ASO is different from a block of internucleotide linkages in a different region of the ASO. In some embodiments, a given ASO of a CPP-ASO comprises modified internucleotide linkages arranged in a gapmer motif. In some embodiments, one or more stereo-controlled nucleotides in an ASO of a CPP-ASO are modified so that a block of stereo-controlled nucleotides in one region of the ASO are different from a block of stereo-controlled nucleotides in a different region of the ASO. In some embodiments, an ASO of a CPP-ASO comprises stereo-controlled nucleotides arranged in a gapmer motif. In some embodiments, an ASO has more than one motif. In some embodiments, an ASO has more than one motif independent of each other.

The CPP-ASO conjugates described herein comprise a CPP conjugated to an ASO. The CPP-ASO conjugate is capable of penetrating a cell membrane so that the CPP-ASO conjugate enters the cytosol of the cell. In some embodiments, more than one CPP is conjugated to an ASO. In some embodiments, a CPP-ASO conjugate comprises 2, 3, 4, or 5 CPPs conjugated to an ASO. In some embodiments, the more than one CPP includes at least two different CPPs. In some embodiments, the more than one CPP is two or more of the same CPPs. In some embodiments, conjugating more than one CPP to an ASO increases ASO activity. In some embodiments, the CPP comprises from four to 40 amino acids. In some embodiments, the CPP has 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40 amino acids.

In some embodiments, the CPP is conjugated directly or indirectly to an ASO. In some embodiments, the CPP is conjugated at the 5′ end of the ASO. In some embodiments, the CPP is conjugated at the 3′ end of the ASO. In some embodiments, the CPP is conjugated at any nucleotide in the ASO. Certain methods of conjugating CPPs are known in the art. In some embodiments, the CPP is conjugated to the ASO at the C-terminus. In some embodiments, the CPP is conjugated to the N-terminus. In some embodiments, the CPP is conjugated to the ASO via a side chain of any amino acid in the CPP. In some embodiments, the CPP is covalently linked to the ASO. In some embodiments, the CPP is chemically conjugated to the ASO. In some embodiments, the CPP is non-covalently linked to the ASO.

In some embodiments, the CPP comprises proteogenic and/or non-proteogenic amino acid(s). Certain exemplary amino acids include alanine, beta-alanine, allo-isoleucine, arginine, asparagine, aspartic acid, cysteine, cyclohexylalanine, 2,3-diaminopropionic acid, 4-fluorophenylalanine, glutamic acid, glutamine, glycine, histidine, homoproline, isoleucine, leucine, lysine, methionine, napthylalanine, norleucine, phenylalanine, phenylglycine, 4-(phosphonodifluoromethyl)phenylalanine, proline, sarcosine, selenocysteine, serine, threonine, tyrosine, tryptophan, valine, tert-butyl-alanine, penicillamine, homoarginine, nicotinyl-lysine, triflouroacetyl-lysine, methyl-leucine, 3-(3-benzothienyl)-alanine, 6-aminohexanoic acid, and 5-aminopentanoic acid. In some embodiments, the CPP contains at least one non-proteogenic amino acid. Certain exemplary non-proteogenic amino acids include allo-isoleucine, beta-alanine, cyclohexylalanine, 2,3-diaminopropionic acid, 4-fluorophenylalanine, homoproline napthylalanine, norleucine, phenylglycine, 4-(phosphonodifluoromethyl)phenylalanine, sarcosine, selenocysteine, tert-butyl-alanine, penicillamine, homoarginine, nicotinyl-lysine, triflouroacetyl-lysine, methyl-leucine, 3-(3-benzothienyl)-alanine, 6-aminohexanoic acid, and 5-aminopentanoic acid. In some embodiments, the at least one non-proteogenic amino acid comprises a modified proline (e.g., a substituted proline). In some embodiments, the CPP contains at least one proteogenic amino acid. Certain exemplary proteogenic amino acids include alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In some embodiments, the CPP contains L- or D-amino acids. In some embodiments, the at least one proteogenic or non-proteogenic amino acid is substituted with one or more substituents. Certain exemplary suitable substituents include halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, and arylthiol groups. In some embodiments, the CPP contains synthetic amino acid mimics, for example, replacement of the peptide amide bond. In some embodiments, the CPP contains a non-natural structure, for example, a non-peptide group that does not contain an amino acid.

In some embodiments, the CPP comprises one or more modified or unmodified arginine residues. In some embodiments, the CPP comprises 1, 2, 3, 4, 5, 6, 7, 8, 9, 10, or 20 arginine residues. In some embodiments, at least 10%, 20%, 30%, 40%, 50%, 60%, 70%, 80%, or 90% of the amino acids of the CPP comprise a modified or unmodified arginine residue.

In some embodiments, the CPP comprises one or more hydrophobic amino acids. Examples of hydrophobic amino acids include glycine, alanine, valine, leucine, isoleucine, methionine, phenylalanine, proline, and tryptophan. In some embodiments, at least one of the one or more hydrophobic amino acids may be substituted with one or more substituents. In some embodiments, the CPP comprises 1, 2, 3, 4, 5, 6, 7, 8, 9, 10, or 20 hydrophobic amino acids. In some embodiments, at least 10%, 20%, 30%, 40%, 50%, 60%, 70%, 80%, or 90% of the amino acids of the CPP comprise a hydrophobic amino acid. In some embodiments, the one or more hydrophobic amino acids comprise one or more aromatic hydrophobic amino acids. Examples of aromatic hydrophobic amino acids include phenylalanine, tryptophan, tyrosine, naphthylalanine, 3-(3-benzothienyl)-alanine, phenylglycine, and homophenylalanine. In some embodiments, at least one of the one or more aromatic hydrophobic amino acids may be substituted with one or more substituents. In some embodiments, the CPP comprises 1, 2, 3, 4, 5, 6, 7, 8, 9, 10, or 20 aromatic hydrophobic amino acids. In some embodiments, at least 10%, 20%, 30%, 40%, 50%, 60%, 70%, 80%, or 90% of the amino acids of the CPP comprise an aromatic hydrophobic amino acid. In some embodiments, the CPP comprises one or more arginine residues and one or more hydrophobic amino acids. In some embodiments, the CPP comprises 1, 2, 3, 4, 5, 6, 7, 8, 9, 10, 15, or 20 arginine residues and 1, 2, 3, 4, 5, 6, 7, 8, 9, 10, 15, or 20 hydrophobic amino acids. In some embodiments, the CPP comprises one or more arginine residues and one or more aromatic hydrophobic amino acids. In some embodiments, the CPP comprises 1, 2, 3, 4, 5, 6, 7, 8, 9, 10, 15, or 20 arginine residues and 1, 2, 3, 4, 5, 6, 7, 8, 9, 10, 15, or 20 aromatic hydrophobic amino acids.

In some embodiments, the CPP comprises one or more modified or unmodified lysine residues. In some embodiments, the CPP comprises 1, 2, 3, 4, 5, 6, 7, 8, 9, 10, or 20 lysine residues.

In some embodiments, the CPP is linear. Certain exemplary linear CPPs include Pip6a peptide (Wood M. J A et al, Mol Therapy—Nucleic Acids, 2012, 1, e38), ApoE peptide (Gait, M. J. et al, Nucleic Acid Therapeutics, 2017, 27, 130), neurotensin-based peptides (Prakash, T. P. et al, J. Med. Chem. 2020, 63, 8471), polyarginine, antennapedia sequences, HIV-TAT, Penetratin, Antp-3A, Buforin II Transportan, MAP (model amphipathic peptide), K-FGF, Ku70, Prion, pVEC, Pep-1, SynB1, Pep-7, HN-1, BGSC (Bis-Guanidium-Spermidine-Cholesterol), and BGTC (Bis-Guanidium-Tren-Cholesterol). In some embodiments, the CPP is a linear peptide having a sequence disclosed in Table 10. In Table 10, B is beta-alanine and X is 6-aminohexanoic acid:

TABLE 10

| | | SEQ |
|---|---|---|
| | | ID |
| CPP | Sequence | No: |
| Compound 11 | RXRRBRRXRRBRXB-azidolysine | 267 |
| Compound 12 | RXRRBRRXRYQFLIRXRBRXRB- | 268 |
| | azidolysine | |
| Compound 13 | Ac-RXRRBRRXRYQFLIRXRBRXRB- | 269 |
| | OH | |
| Compound 14 | Ac-LRKLRKRLLRXB-azidolysine | 270 |
| Compound 15 | K(azidoacetyl)-LYENKPRRPYIL | 271 |
| Compound 16 | K(azidoacetyl)- | 272 |
| | PPPAGSSPGLYENKPRRPYIL | |

In some embodiments, the CPP is a cyclic cell penetrating peptide (“cCPP”). Certain exemplary cCPPs include CPP9, CPP12 (Pei, D. et al. Biochemistry, 2016, 55, 2601) and others as outlined in Tiwari, K. et al. Mol. Pharmaceutics 2019, 16, 9, 3727. In some embodiments, a cyclic CPP of a CPP-ASO has one of the following structures:

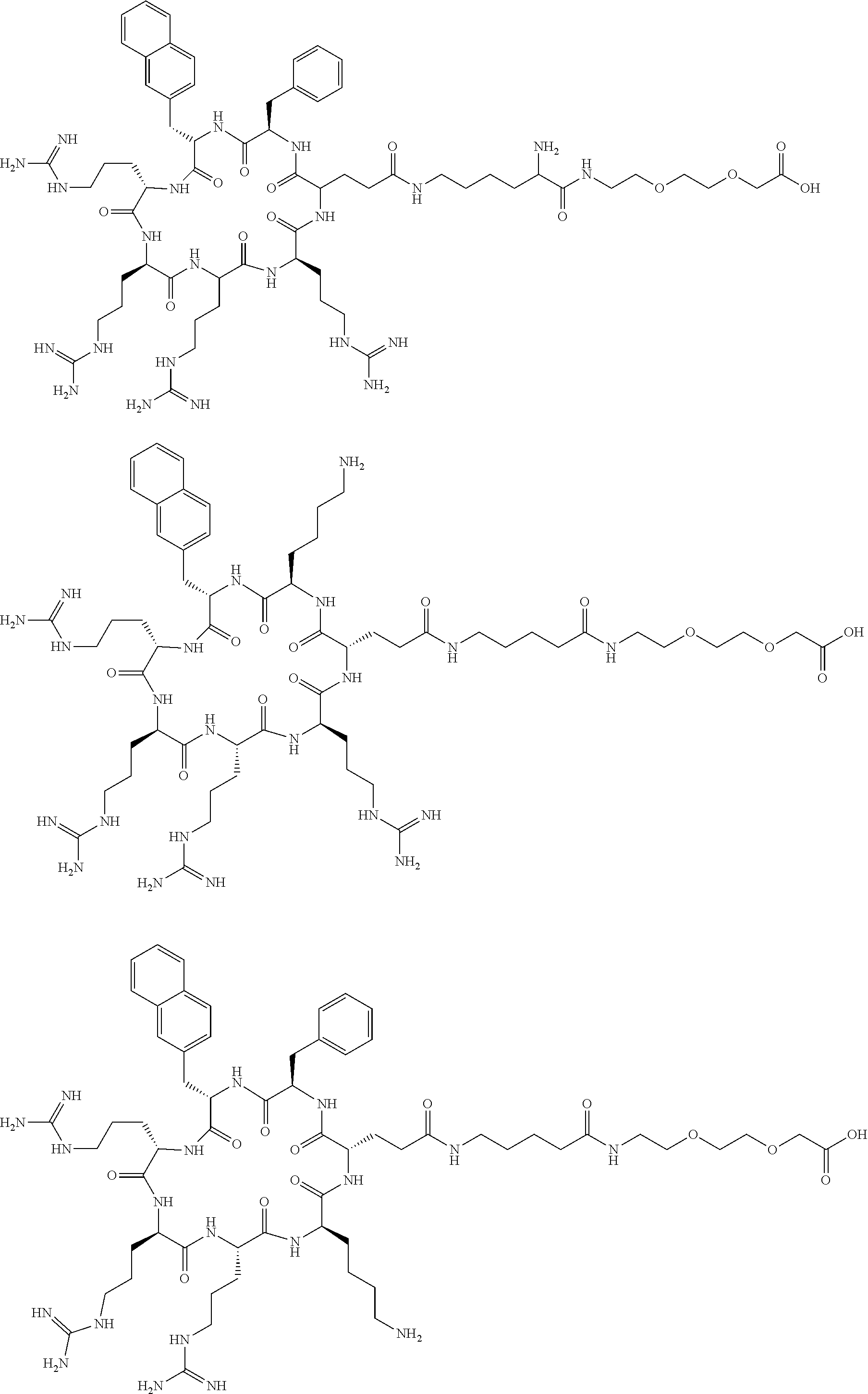

In some embodiments, the CPP is synthesized using a solid-phase approach and/or a solution phase approach. In some embodiments, the CPP is synthesized using both a solid phase and solution phase approach where part of the synthesis occurs using a solid phase and another part of the synthesis occurs using a solution phase. Further information about synthesis of certain CPPs according to some embodiments is included in the Examples below.

In some embodiments, the CPP comprises a lipoic acid group. In some embodiments, the lipoic acid moiety is an (R)-lipoic acid group. In some embodiments, the lipoic acid moiety is an (S)-lipoic acid group. In some embodiments, the lipoic acid group is conjugated to a lysine residue of the CPP. In some embodiments, the lipoic acid group is conjugated to a non-lysine residue (e.g., a 5-aminopentanoic acid) of the CPP. In some embodiments, the CPP comprises one to five lipoic acid groups. In some embodiments, the CPP comprises one, two, three, four, or five lipoic acid groups.

In some embodiments, a cyclic CPP of a CPP-ASO has one of the following structures:

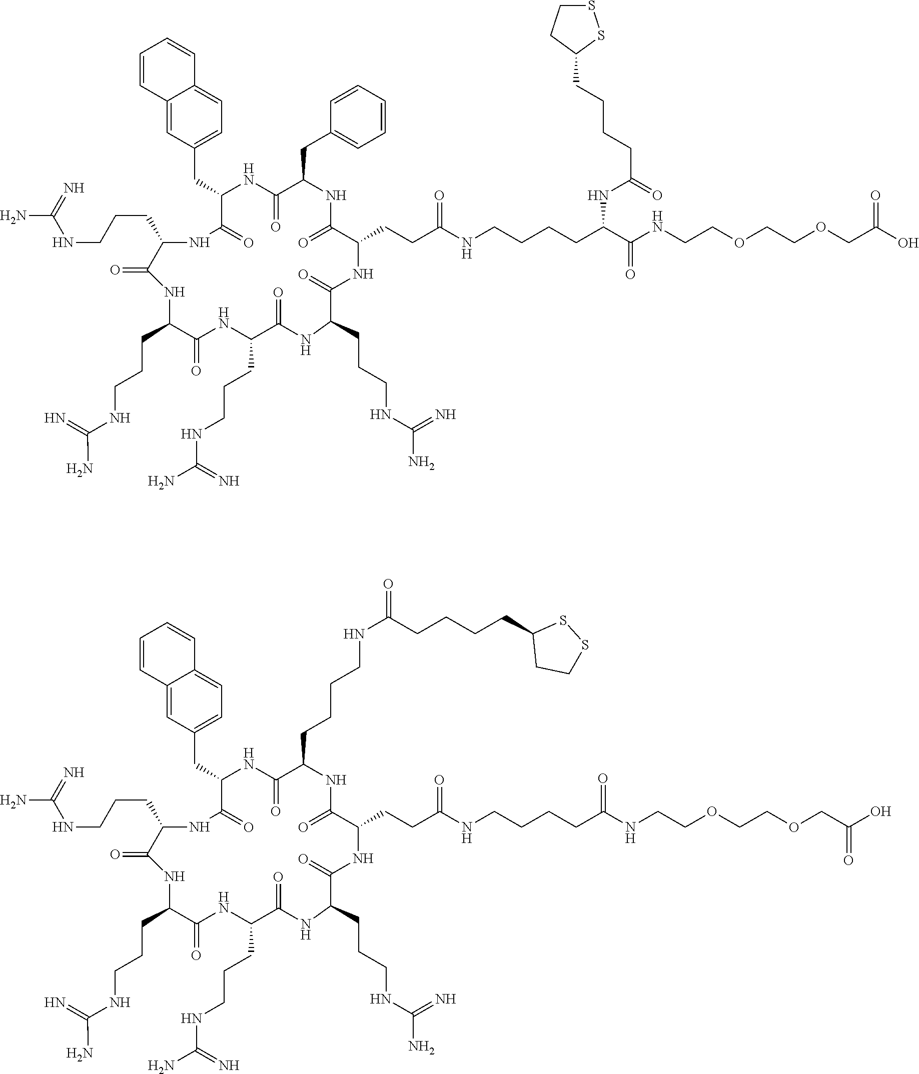 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCC[C@H](NC(=O)CCCC[C@@H]2CCSS2)C(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O ; N=C(N)NCCC[C@@H]1NC(=O)[C@H](Cc2ccc3ccccc3c2)NC(=O)[C@@H](CCCCNC(=O)CCCC[C@@H]2CCSS2)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

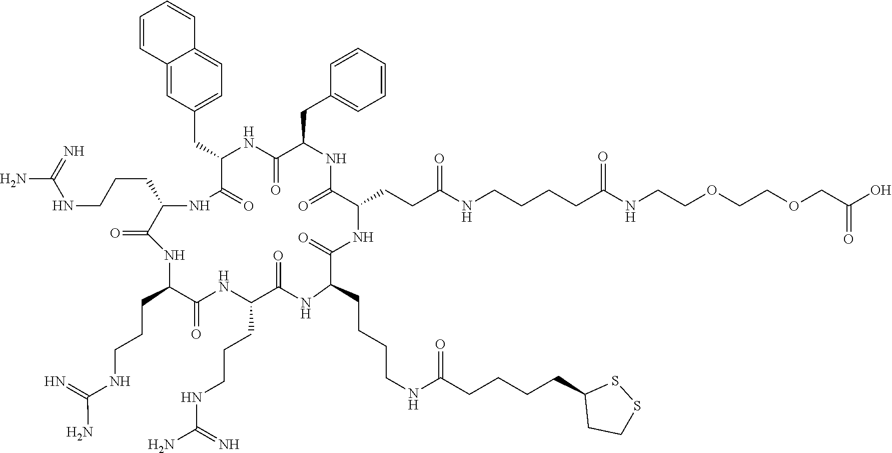 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCCNC(=O)CCCC[C@@H]2CCSS2)NC(=O)[C@H](CCCNC(=N)N)NC(=O)[C@@H](CCCNC(=N)N)NC1=O]

In some embodiments, a CPP comprising a lipoic acid group may be synthesized according to an exemplary synthesis scheme described in the Examples below.

In some embodiments, the CPP comprises one or more lactam amino acids. In some embodiments, the CPP comprises 1, 2, 3, 4, or 5 lactam amino acids. In some embodiments, each lactam amino acid is independently an 8, 9, or 10-membered ring.

In some embodiments, the one or more lactam amino acids each independently have a structure according to Formula III:

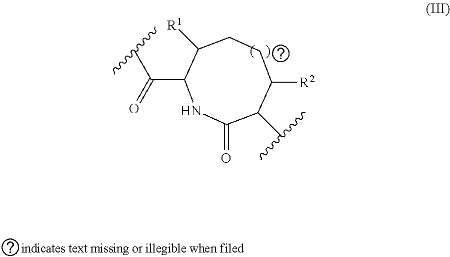

wherein R¹ and R² are each independently selected from the group consisting of H, an aryl group, a heteroaryl group, an alkylaryl group, an arylalkyl group, a linear or branched alkyl group, and a guanidine-comprising group, each of which may be independently substituted with one or more substituents, and wherein n is an integer from 1 to 3.

In some embodiments, R¹ and/or R² comprise an aryl group. In some embodiments, R¹ and R² each independently comprise an aryl group. The aryl group may be monocyclic or polycyclic. Certain exemplary monocyclic aryl groups include phenyl and benzyl groups. An example of a polycyclic aryl group is a naphthyl group. In some embodiments, R¹ and/or R² comprise a guanidine-comprising group. In some embodiments, R¹ and R² each independently comprise a guanidine-comprising group. An example of a guanidine-comprising group is (CH₂)₂CN₃H₄.

In some embodiments, R¹ and R² are H. In some embodiments, R¹ is H and R² is phenyl. In some embodiments, R¹ is phenyl and R² is H. In some embodiments, R¹ and R² are phenyl. In some embodiments, R¹ is 2-naphthyl and R² is H. In some embodiments, R¹ is H and R² is 2-naphthyl. In some embodiments, R¹ and R² are 2-naphthyl. In some embodiments, R¹ is 2-naphthyl and R² is phenyl. In some embodiments, R¹ is (CH₂)₂CN₃H₄ and R² is H. In some embodiments, R¹ is H and R² is (CH₂)₂CN₃H₄. In some embodiments, R¹ is (CH₂)₂CN₃H₄ and R² is 2-naphthyl. In some embodiments, R¹ and R² are (CH₂)₂CN₃H₄.

In some embodiments, n is 1. In some embodiments, n is 2. In some embodiments, n is 3.

In some embodiments, a CPP comprising a lactam amino acid has a structure shown below:

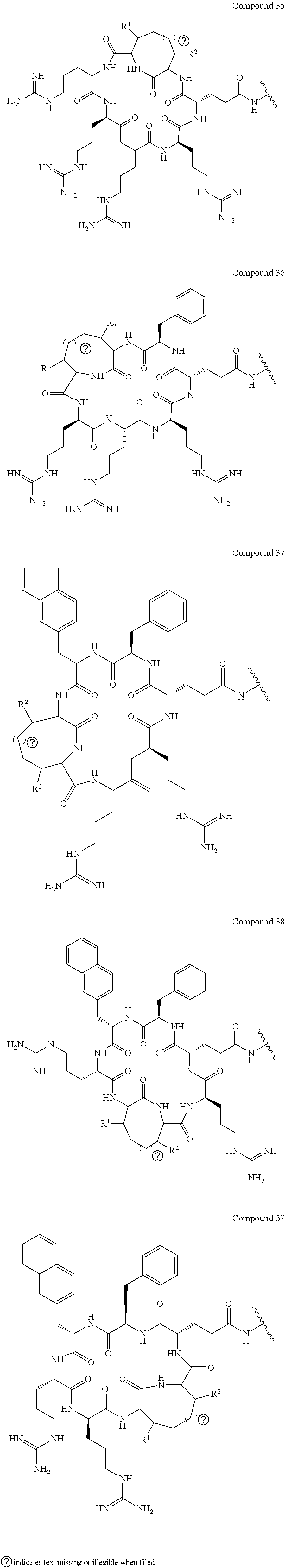

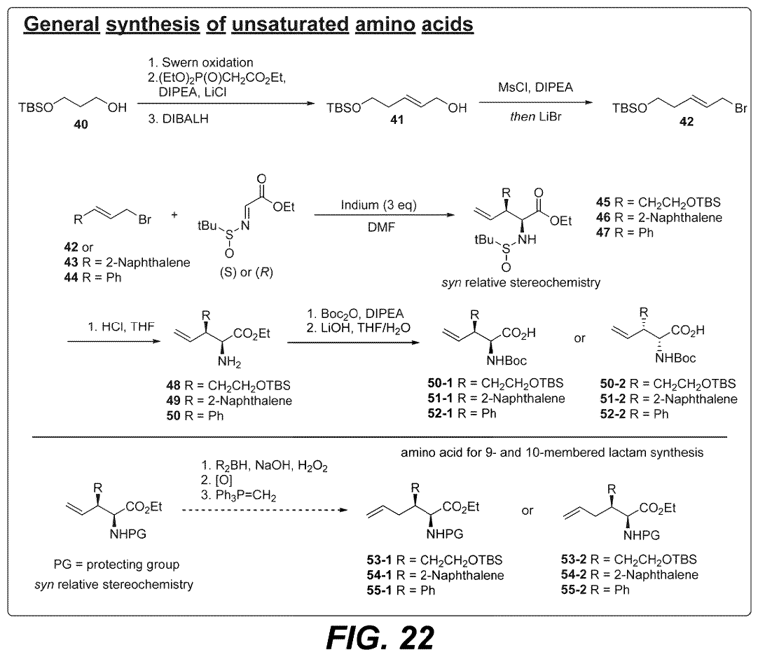

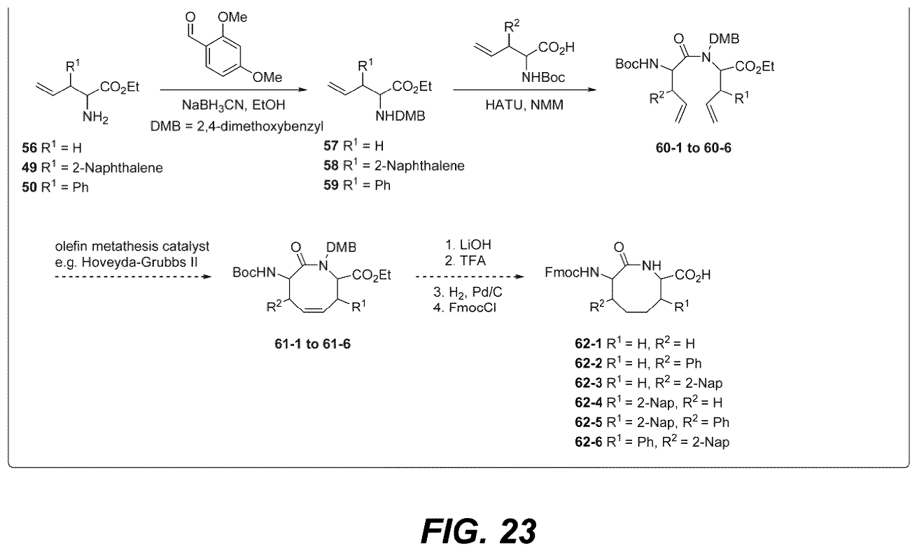

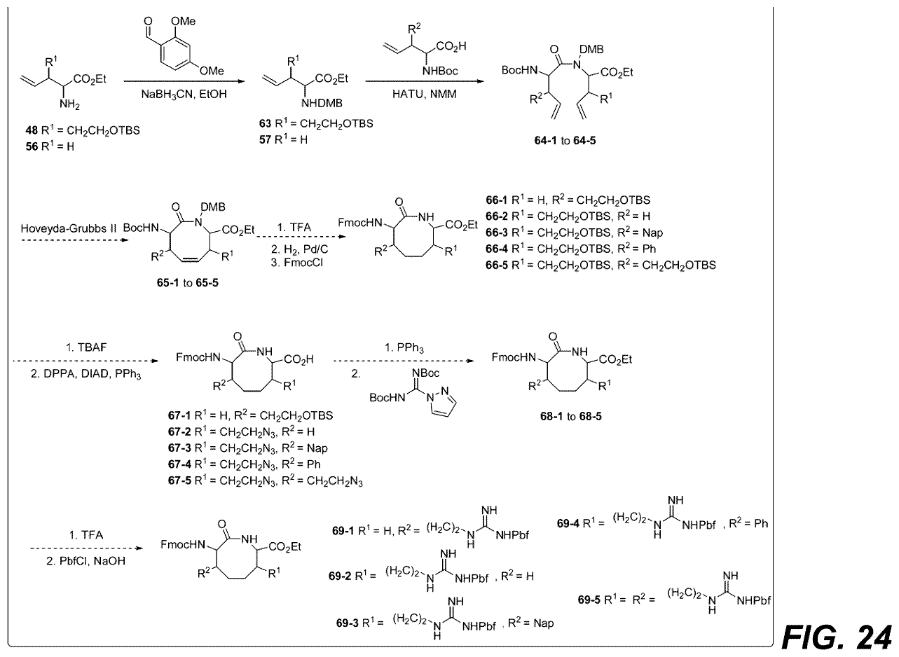

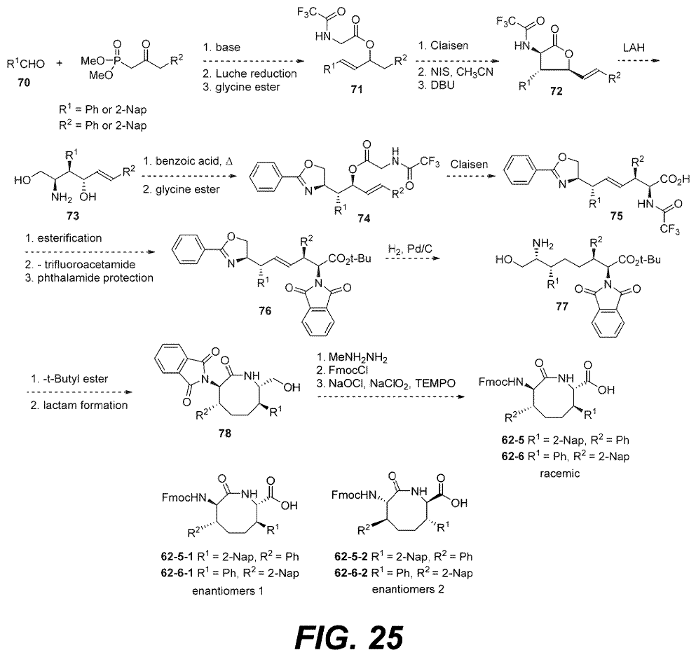

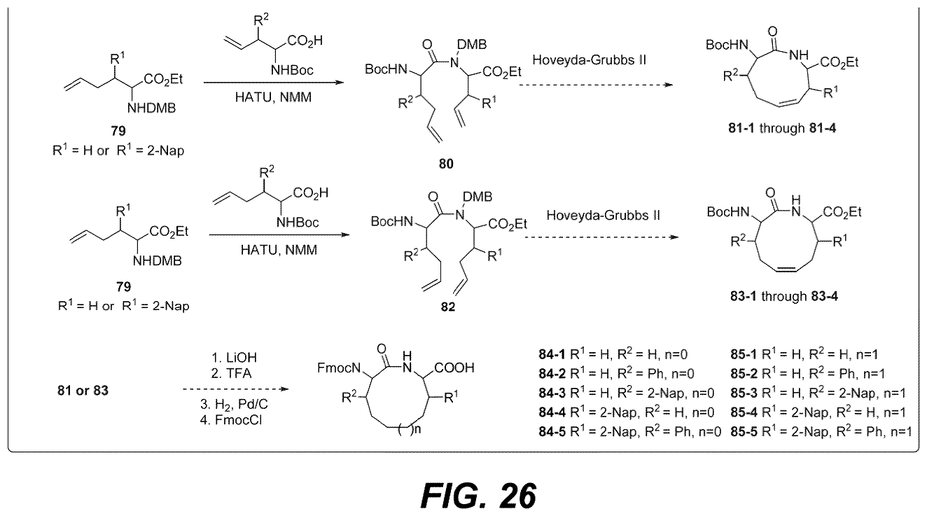

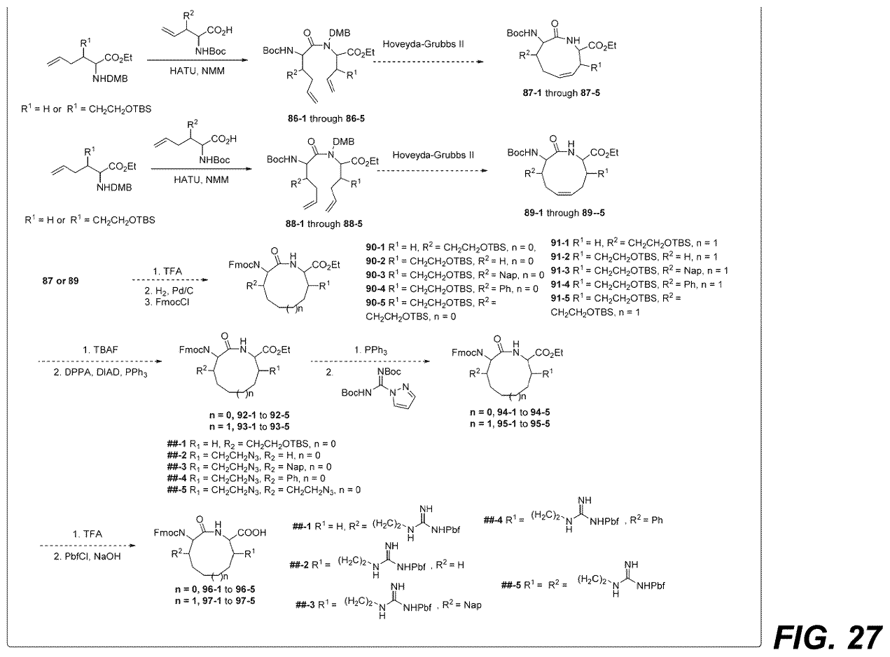

In some embodiments, a CPP comprising a lactam amino acid may be synthesized according to an exemplary synthesis scheme shown in FIGS. 22-27 and the Examples below.

In some embodiments, the CPP comprises one or more modified proline residues. In some embodiments, the one or more modified proline residues comprise one or more substituents. Certain exemplary suitable substituents include an aryl group and a guanidine-comprising group. Examples of suitable aryl groups include phenyl, benzyl, and naphthyl groups. An example of a suitable guanidine-comprising group includes, but is not limited to, —(CH₂)₂—CN₃H₄.

In some embodiments, the one or more modified proline residues of the CPP each independently have a structure according to Formula IV:

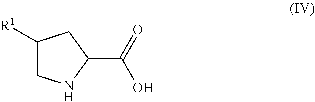

wherein R¹ is an aryl group or a guanidine-comprising group. In some embodiments, R¹ is a guanidine-comprising group. In some embodiments, R¹ is —(CH₂)₂—CN₃H₄. In some embodiments, R¹ is an aryl group. In some embodiments, R¹ is benzyl. In some embodiments, R¹ is phenyl. In some embodiments, R¹ is naphthyl.

In some embodiments, a CPP comprising a modified proline residue has a structure shown below:

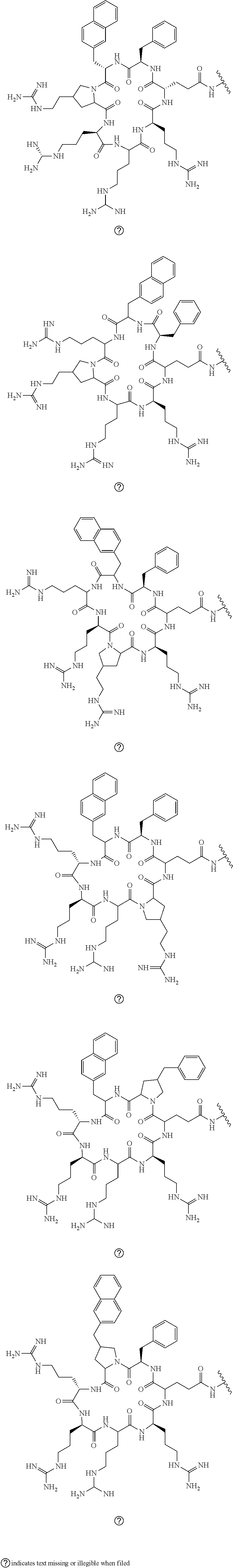

In some embodiments, a CPP comprising a modified proline residue may be synthesized according to an exemplary synthesis scheme shown in the Examples below.

In some embodiments, the CPP comprises one or more oxadiazole linkages. In some embodiments, the one or more oxadiazole linkages have a structure according to Formula V:

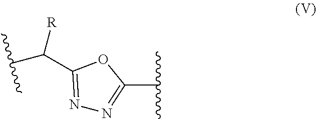

where R is a substituted or unsubstituted aryl group. Certain exemplary suitable aryl groups include phenyl, benzyl, naphthyl, and methyl naphthyl.

In some embodiments, a CPP comprising an oxadiazole linkage has a structure according to Formula VI:

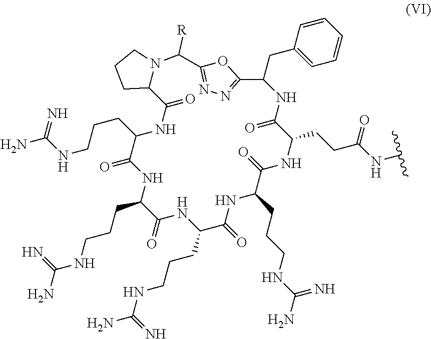

where R is a substituted or unsubstituted aryl group. In some embodiments, R is phenyl, benzyl, naphthyl, or methyl naphthyl.

In some embodiments, a CPP comprising an oxadiazole linkage may be synthesized according to an exemplary synthesis scheme shown in the Examples below.

In some embodiments, a CPP-ASO conjugate further comprises a nuclear localization sequence (NLS). A nuclear localization sequence generally refers to an amino acid sequence that facilitates transport of molecules comprising the sequence into the nucleus of eukaryotic cells. The nuclear localization sequence may be a monopartite or bipartite nuclear localization sequence. Examples of suitable nuclear localization sequences include sequences comprising all or a portion of one or more of the following sequences: PKKKRKV (SEQ ID NO: 273) from simian virus 40 (SV40), PKLKRQ (SEQ ID NO: 274), RPRK (SEQ ID NO: 275), RRARRPRG (SEQ ID NO: 276), KRPAATKKAGQAKKKK (SEQ ID NO: 277) from nucleoplasmin, PAAKRVKLD (SEQ ID NO: 278) and RQRRNELKRSP (SEQ ID NO: 279) from c-myc, RMRKFKNKGKDTAELRRRRVEVSVELRKAKKDEQILKRRNV (SEQ ID NO: 280) from the IBB domain of importin-alpha, VSRKRPRP (SEQ ID NO: 281) and PPKKARED (SEQ ID NO: 282) from polyomavirus large T, PQPKKKPL (SEQ ID NO: 283) from human p53, SALIKKKKKMAP (SEQ ID NO: 284) from mouse c-abl IV, DRLRR (SEQ ID NO: 285) and PKQKKRK (SEQ ID NO: 286) from the influenza virus NS1, RKLKKKIKKL (SEQ ID NO: 287) from the Hepatitis virus delta antigen, REKKKFLKRR (SEQ ID NO: 288) from the mouse Mxl protein, KRKGDEVDGVDEVAKKKSKK (SEQ ID NO: 289) from the human poly(ADP-ribose) polymerase, and RKCLQAGMNLEARKTKK (SEQ ID NO: 290) from the steroid hormone receptors (human) glucocorticoid. Additional examples of nuclear localization sequences are described in Lu, J. et al., Types of nuclear localization signals and mechanisms of protein import into the nucleus. Cell Commun Signal 19, 60 (2021), which is incorporated by reference herein in its entirety.

In some embodiments, the NLS is covalently or non-covalently coupled to the CPP, ASO, and/or linker of a CPP-ASO. In some embodiments, the NLS is covalently or non-covalently coupled to the CPP of a CPP-ASO. In some instances, the CPP is a linear peptide. In some instances, the CPP is a cyclic peptide. In some embodiments, the NLS is covalently or non-covalently coupled to the ASO of a CPP-ASO. In some embodiments, the NLS is covalently or non-covalently coupled to a linker coupling a CPP and an ASO.

### Manufacturing Antisense Oligonucleotides

The antisense molecules used in accordance with this disclosure may be made through well-known techniques of solid phase synthesis. Equipment for such synthesis is available from several sources including, for example, Applied Biosystems (Foster City, Calif.). One method for synthesizing oligonucleotides on a modified solid support is described in U.S. Pat. No. 4,458,066.

Any other methods for such synthesis known in the art may additionally or alternatively be employed. It is well known to use similar techniques to prepare oligonucleotides, such as phosphorothioates and alkylated derivatives. In one such automated embodiment, diethyl-phosphoramidites are used as starting materials and may be synthesized as described by Beaucage, et al., Tetrahedron Letters, 22:1859-1862 (1981).

In some embodiments, the ASOs are synthesized in a way so that all nucleotides of the ASO are stereopure.

In some embodiments, the ASOs are synthesized in vitro and do not include antisense compositions of biological origin. In some embodiments, the ASOs may also be mixed, encapsulated, conjugated or otherwise associated with other molecules, molecule structures, or mixtures of compounds, as for example, liposomes, lipids, receptor targeted molecules for assisting in uptake, distribution and/or absorption.

### Conjugating Cell Penetrating Peptides to Antisense Oligonucleotides

In some embodiments, the antisense oligonucleotides are conjugated to cell penetrating peptides using known chemical reactions. Examples are found in: Gait. M. J. et al. Curr. Pharm. Des. 2005, 11, 3639; Prescher, J. A. et al. Nat. Rev. Chem. 2020, 4, 476; Jeon, J. et al. Molecules, 2019, 24, 3567; Stetsenko, D. Molecules, 2021, 26, 5420.

In some embodiments, the CPP is conjugated to the ASO via strain-promoted azide-alkyne cycloaddition reaction (“click chemistry”). In some embodiments, the CPP is conjugated to the ASO via strained alkene-tetrazine cycloaddition reaction. In some embodiments, the CPP is conjugated to the ASO by an amide bond. In some embodiments, the CPP is conjugated to the ASO using one of the bonds in the image below:

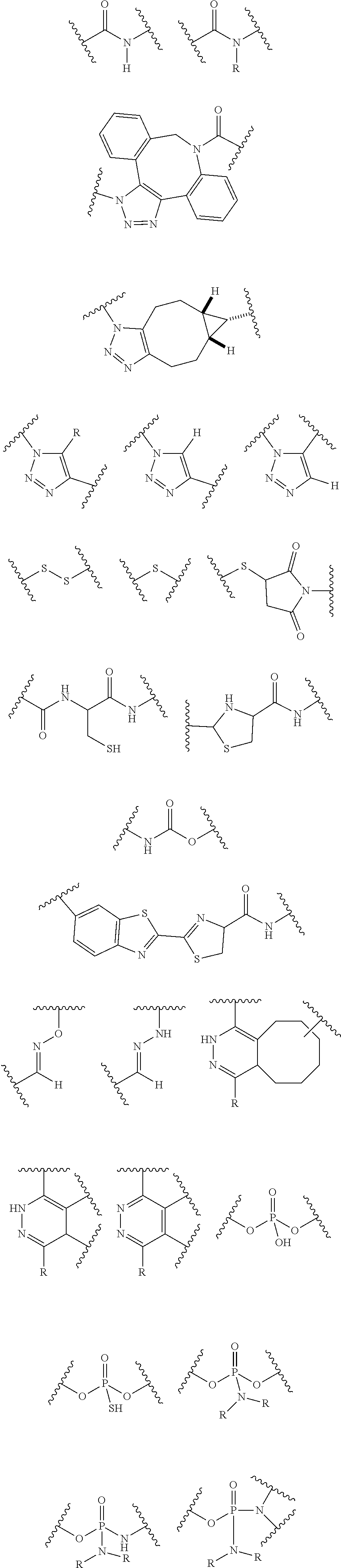

In some embodiments, the CPP is conjugated directly to the ASO. In some embodiments, the CPP is indirectly conjugated to the ASO with a linker between the CPP and ASO. In some embodiments, the linker comprises an alkyl group, a carbocyclic group, a heterocyclic group, a polyethylene glycol, or one or more of these groups. In some embodiments, the linker comprises one or more proteogenic or non-proteogenic amino acids. In some embodiments, the one or more proteogenic or non-proteogenic amino acids comprise sarcosine. In some embodiments, the linker is a cleavable linker. Certain exemplary suitable cleavable linkers include linkers comprising valine-citrulline (“Val-Cit”), valine-alanine (“Val-Ala”), glutamic acid-valine-citrulline (“Glu-Val-Cit”), and/or alanine-alanine-asparagine (“Ala-Ala-Asn”).

### Methods of Inducing Exon-2 Skipping During Pre-mRNA Splicing

In some embodiments, the CPP-ASOs are used to induce Exon-2 skipping during processing of CD33 pre-mRNA. In some embodiments, at least one CPP-ASO disclosed herein is used to induce Exon-2 skipping in CD33 pre-mRNA during pre-mRNA splicing. In some embodiments, the at least one CPP-ASO is introduced into a cell, wherein the at least one CPP-ASO is complementary to all or a portion of SEQ ID NO:1, wherein the CPP-ASO hybridizes to a target region of the CD33 gene, and wherein the CPP-ASO induces Exon-2 skipping during pre-mRNA splicing of the CD33 gene. In some embodiments, the CPP-ASO administered to induce Exon-2 skipping during pre-mRNA splicing comprises one of SEQ ID NOS: 2-10. In some embodiments, the CPP-ASO administered to induce Exon-2 skipping during pre-mRNA splicing comprises one of SEQ ID NOS:2-15, 36-39, 82, 83, 96, 97, 128, 132, 135, 136, 183, 184, 190, 196, 197, 202, 224, or 252. In some embodiments, the CPP-ASO administered to induce Exon-2 skipping during pre-mRNA splicing comprises one of SEQ ID NOS: 2, 12, 224, or 252.

In some embodiments, a CPP-ASO can be introduced by transfection along with one or more transfection agents. In some embodiments, excipients or transfection agents are capable of forming complexes, nanoparticles, micelles, vesicles, and/or liposomes that help deliver each CPP-ASO complexed or trapped in a vesicle or liposome through a cell membrane. Many of these excipients are known in the art. Suitable excipients or transfection agents include LipofectAMINE™ 2000 (Invitrogen), Endo-Porter peptide, polyethylenimine (PEI; ExGen500 (MBI Fermentas)), or derivatives thereof, or similar cationic polymers, including polypropyleneimine or polyethylenimine copolymers (PECs) and derivatives, synthetic amphiphiles (SAINT-18), Lipofectin™, DOTAP and/or viral capsid proteins that are capable of self-assembly into particles that can be used when delivering a CPP-ASO to a cell. Their high transfection potential is combined with an expected low to moderate toxicity in terms of overall cell survival. The ease of structural modification can be used to allow further modifications and the analysis of their further (in vivo) nucleic acid transfer characteristics and toxicity.

### Therapeutic Methods

Disclosed herein are methods of treating a subject having a neurodegenerative disease comprising administering at least one CPP-ASO disclosed herein. In some embodiments, the methods comprise administering a therapeutically effective amount of at least one CPP-ASO disclosed herein. In some embodiments, the methods comprise administering a therapeutically effective amount of at least one CPP-ASO that hybridizes to all or a portion of SEQ ID NO:1. In some embodiments, the methods comprise administering a therapeutically effective amount of at least one CPP-ASO comprising one of SEQ ID NOS:2-10. In some embodiments, the methods comprise administering a therapeutically effective amount of at least one CPP-ASO comprising one of SEQ ID NOS:2-15, 36-39, 82, 83, 96, 97, 128, 132, 135, 136, 183, 184, 190, 196, 197, 202, 224, or 252. In some embodiments, the methods comprise administering a therapeutically effective amount of at least one CPP-ASO comprising one of SEQ ID NOS: 2, 12, 224, or 252. In some embodiments, the neurodegenerative disease is characterized by a mutation in the CD33 gene. In some embodiments, the neurodegenerative disease is characterized by an aberrant microglial phenotype. In some embodiments, the neurodegenerative disease is Alzheimer's Disease, microfibromialgia, or multiple sclerosis.

In some embodiments, the CPP-ASO administered to a subject having a neurodegenerative disease may be administered in a pharmaceutical composition. In some embodiments, the amount of CPP-ASO administered in a pharmaceutical composition may be dependent on the subject being treated, the subject's weight, the manner of administration, and the judgment of the prescribing physician. For example, in some embodiments, a dosing schedule may involve the daily or semi-daily administration of the pharmaceutical composition at a perceived dosage of about 1 μg to about 1000 mg. In some embodiments, intermittent administration, such as on a weekly, monthly, quarterly, or yearly basis, of a dose of the pharmaceutical composition may be employed. In accordance with standard dosing regimens, in some embodiments, physicians will readily determine optimum dosages and will be able to readily modify administration to achieve such dosages.

A therapeutically effective amount of a compound or composition disclosed herein can be measured by the therapeutic effectiveness of the compound. In some embodiments, the dosages, however, may be varied depending upon the requirements of the patient, the severity of the condition being treated, and the compound being used. In some embodiments, the therapeutically effective amount of a disclosed compound is sufficient to establish a maximal plasma concentration. In some embodiments, preliminary doses as, for example, determined according to animal tests, and the scaling of dosages for human administration is performed according to art accepted practices.

In some embodiments, toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. In some embodiments, compositions that exhibit large therapeutic indices are desirable.

In some embodiments, data obtained from the cell culture assays or animal studies can be used in formulating a range of dosage for use in humans. In some embodiments, therapeutically effective dosages achieved in one animal model may be converted for use in another animal, including humans, using conversion factors known in the art (see, e.g., Freireich et al., Cancer Chemother. Reports 50(4):219244 (1966).

The CPP-ASOs herein may be administered in a pharmaceutical composition comprising therapeutically effective amounts of an CPP-ASO together with pharmaceutically acceptable excipients, diluents, preservatives, solubilizers, emulsifiers, adjuvants and/or carriers. In some embodiments, such compositions include diluents of various buffer content (e.g., Tris-HCl, acetate, phosphate), pH, and ionic strength, and additives such as detergents and solubilizing agents (e.g., Tween 80, Polysorbate 80), anti-oxidants (e.g., ascorbic acid, sodium metabisulfite), preservatives (e.g., Thimersol, benzyl alcohol), and bulking substances (e.g., lactose, mannitol). In some embodiments, the material may be incorporated into particulate preparations of polymeric compounds such as polylactic acid, polyglycolic acid, etc. or into liposomes. In some embodiments, Hyaluronic acid may also be used. Such compositions may influence the physical state, stability, rate of in vivo release, and/or rate of in vivo clearance of the present CPP-ASOs and derivatives. In some embodiments, the compositions may be prepared in liquid form, or may be in dried powder, such as lyophilized form.

### Administration

In some embodiments, a pharmaceutical composition comprising a CPP-ASO and a pharmaceutically acceptable carrier or excipient may be prepared for administration according to techniques well known in the pharmaceutical industry. In some embodiments, such techniques include combining the CPP-ASO with the carrier and/or excipient(s) into association in a unit dosage form.

In some embodiments, compositions suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. In some embodiments, such formulations may be prepared by any suitable method which includes the step of bringing into association at least one embodiment of the present disclosure as the active compound and at least one carrier or excipient (which may constitute one or more accessory ingredients). In some embodiments, the at least one carrier is acceptable in the sense of being compatible with the other ingredients of the formulation and is not deleterious to the recipient. In some embodiments, the carrier may be a solid or a liquid, or both, and may be formulated with at least one compound described herein as the active compound in a unit-dose formulation, for example, a tablet, which may contain from about 0.05% to about 95% by weight of the at least one active compound. In some embodiments, other pharmacologically active substances may also be present including other compounds. In some embodiments, the formulations of the present disclosure may be prepared by any of the well-known techniques of pharmacy consisting essentially of admixing the components.

For solid compositions, in some embodiments, conventional nontoxic solid carriers include, for example, pharmaceutical grades of mannitol, lactose, starch, magnesium stearate, sodium saccharin, talc, cellulose, glucose, sucrose, magnesium carbonate, and the like. In some embodiments, liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, in some embodiments, suitable formulations may be prepared by uniformly and intimately admixing the at least one active compound of the present disclosure with a liquid or finely divided solid carrier, or both, and then, if desired, shaping the product. For example, in some embodiments, a tablet may be prepared by compressing or molding a powder or granules of at least one embodiment of the present disclosure, which may be optionally combined with one or more accessory ingredients. In some embodiments, compressed tablets may be prepared by compressing, in a suitable machine, at least one embodiment of the present disclosure in a free-flowing form, such as a powder or granules, which may be optionally mixed with a binder, lubricant, inert diluent and/or surface active/dispersing agent(s). In some embodiments, molded tablets may be made by molding, in a suitable machine, where the powdered form of at least one embodiment of the present disclosure is moistened with an inert liquid diluent.

In some embodiments, formulations suitable for buccal (sub-lingual) administration include lozenges comprising at least one embodiment of the present disclosure in a flavored base, for example, sucrose and acacia or tragacanth, and pastilles comprising the at least one compound in an inert base such as gelatin and glycerin or sucrose and acacia.

In some embodiments, formulations suitable for parenteral administration comprise sterile aqueous preparations of at least one embodiment of the present disclosure, which are approximately isotonic with the blood of the intended recipient. In some embodiments, these preparations are administered intravenously, although administration may also be affected by subcutaneous, intramuscular, intraperitoneal, intracerebroventricular, or intradermal injection. In some embodiments, these preparations are administered via osmotic pump. In some embodiments, such preparations may conveniently be prepared by admixing at least one embodiment described herein with water and rendering the resulting solution sterile and isotonic with the blood. In some embodiments, injectable compositions according to the present disclosure may contain from about 0.1 to about 5% w/w of the active compound.

In some embodiments, formulations suitable for rectal administration are presented as unit-dose suppositories. In some embodiments, these may be prepared by admixing at least one embodiment as described herein with one or more conventional solid carriers, for example, cocoa butter, and then shaping the resulting mixture.

In some embodiments, formulations suitable for topical application to the skin may take the form of an ointment, cream, lotion, paste, gel, spray, aerosol, or oil. In some embodiments, carriers and excipients which may be used include Vaseline, lanoline, polyethylene glycols, alcohols, and combinations of two or more thereof. In some embodiments, the CPP-ASO is generally present at a concentration of from about 0.1% to about 15% w/w of the composition, for example, from about 0.5 to about 2%.

### EXAMPLES

The following Examples serve to more fully describe the invention. They are meant for illustrative purposes and are not meant to limit the invention in any way.

### ABBREVIATIONS

ASO: antisense oligonucleotide
CPP-ASO: cell penetrating peptide conjugated to an antisense oligonucleotide
DNA: deoxyribonucleic acid
CPP: cell penetrating peptide
cDNA: complementary deoxyribonucleic acid
RNA: ribonucleic acid
mRNA: messenger ribonucleic acid
PMO: phosphorodiamidate morpholino oligomer
MOE: methoxyethyl
LOAD: late onset Alzheimer's Disease
SNP: single nucleotide polymorphism
PNA: peptide nucleic acid
DOTAP: 1,2 dioleoyl 3 trimethylammoniopropane
PEI: polyethylenimine
PEC: polyethylenimine copolymers
HRMS: high resolution mass spectrometry
HATU: Hexafluorophosphate Azabenzotriazole Tetramethyl Uronium
MW: molecular weight
SP: stereopure
UPLC: ultra performance liquid chromatography
MS: mass spectrometry
MTBE: Methyl tert-butyl ether
DCM: dichloromethane
DMB: 2,4-dimethoxybenzyl
DIPEA: N,N-diisopropylethylamine
TFA: trifluoroacetic acid
THF: tetrahydrofuran
RT: room temperature
H: hour
Min: minute
EA or EtOAc: ethyl acetate
HPRT1: hypoxanthine phosphoribosyltransferase 1
GAPDH1: glyceraldehyde 3 phosphate dehydrogenase 1
NTC: non-targeting control
WP: well plate
Bz: benzoyl
Cbz: benzyloxycarbonyl
CE: 2-cyanoethyl
Trt: trityl
IPr: isopropyl
Sar: Sarcosine
ESI-TOF-MS: electrospray ionization—time-of-flight mass spectrometry

### Example 1: Reducing or Interfering with Full Length CD33

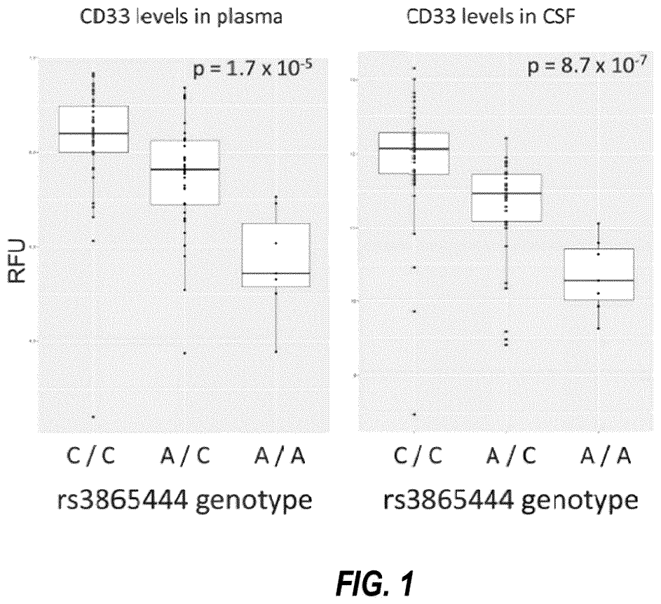

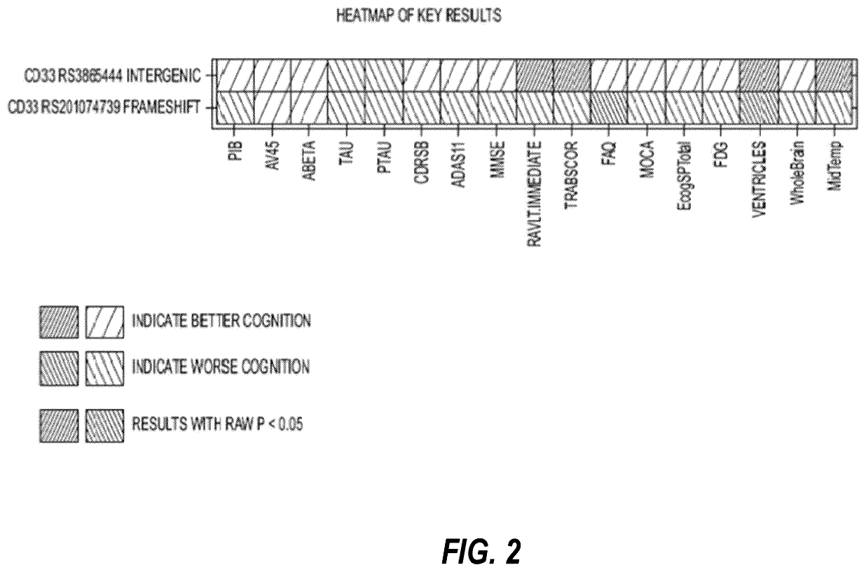

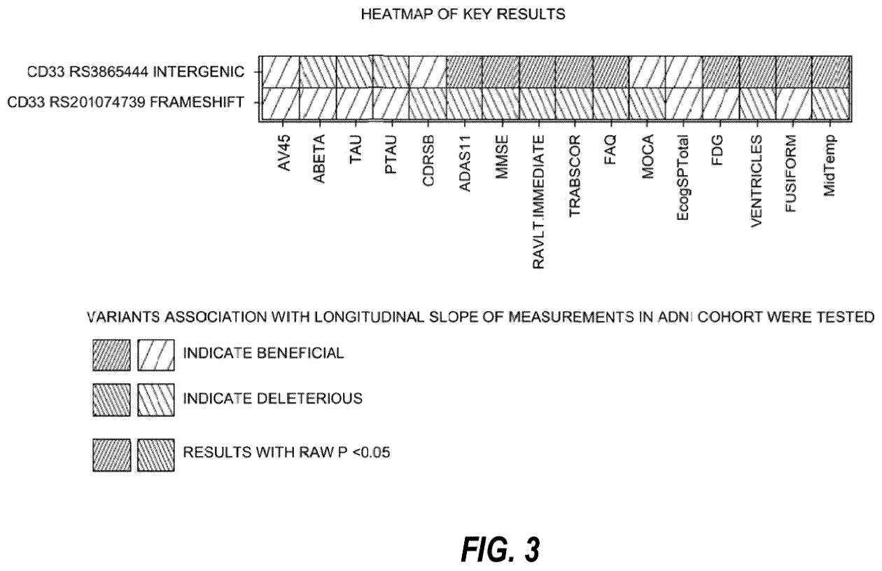

SNP rs3865444 was reported to be associated with an increased skipping of Exon-2 of CD33 and with reduced levels of full length CD33 on the surface of monocytes. The allele was found to be associated with decreased levels of full length CD33 in human cerebrospinal fluid (CSF) and plasma when measured using Somascan technology (FIG. 1). In a study by the Alzheimer's Disease Neuroimaging Initiative (ADNI), the allele was found to be associated with decreased ventricle volume and increased midtemporal volume, which are both consistent with protection against Alzheimer's Disease (FIG. 2). Moreover, in the longitudinal analysis, the allele was associated with improved slope for Alzheimer's Disease Assessment Scale (ADAS) 11, mini-mental state examination (MMSE), Rey Auditory Verbal Learning Test (RAVLT) immediate, Trial Making TestB (TRABSCOR), Functional Activities Questionnaire (FAQ), ¹⁸F-fluorodeoxyglucose-positron emission tomography (FDG PET), ventricle volume, fusiform gyrus, and midtemporal volume (FIG. 3), indicating protection against the disease.

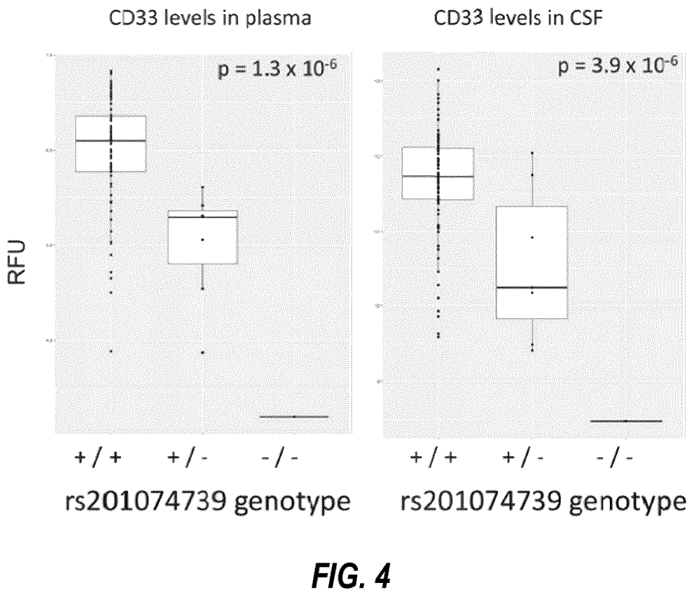

On the other hand, rs201074739 is a 4-base pair deletion in exon3 of the CD33 gene. This causes a frameshift in the open reading frame and a premature translation termination. The indel was associated with decreased levels of full length CD33 in human CSF and plasma when measured using SomaScan technology (FIG. 4). However, this indel has not been associated with a reduced risk of the disease so far. Moreover, it was associated with increased ventricle volume and a worse functional activities questionnaire (FAQ) score, suggesting a deleterious effect (FIG. 2).

Accordingly, successfully inducing Exon-2 skipping of CD33 may have therapeutic benefits.

### Example 2: General ASO Formulas

PMO oligonucleotides were designed for screening. The designed oligonucleotides listed in Tables 11 and 12 below were made by GeneTools LLC (www.qene-tools.com). Table 11 lists the top PMO oligonucleotides with their deconvoluted MS data. Table 1 includes the top PMO oligonucleotides in Table 11, as well as other PMO oligonucleotides. All PMO oligonucleotides listed in Tables 11 and 1 contain a phosphorodiamidate-attached sarcosine linker (Sar) at the 5′ end. All PMO oligonucleotides in Tables 11 and 1 were synthesized with unmodified cytosine PMO nucleotide. All PMO oligonucleotides listed in Tables 11 and 1 have stereorandom internucleotide linkages, and thus are called stereorandom PMO oligonucleotides. The general formula of the PMO oligonucleotides listed in Tables 11 and 1 is:

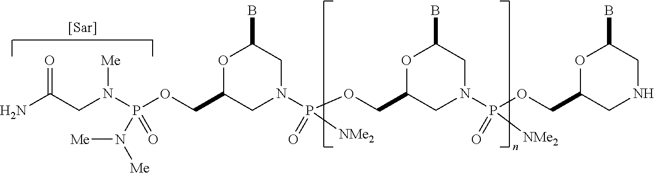

### General Formula for Stereorandom PMO Oligonucleotides

TABLE 11

| | | | MW | |
|---|---|---|---|---|
| | | | (with | |
| | | SEQ ID | Sarcosine | |
| ASO # | Seq. | NO | linker) | MS observed |
| PMO-002 | 5′ -CCTCACCTGTCACATGCACAGAGAG-3′ | 2 | 8398.06 | 8403.0 |
| PMO-003 | 5′ -CCTGTCACATGCACAGAGAGCTGGG-3′ | 3 | 8494.11 | 8498.4 |
| PMO-004 | 5′ -ATTTGGTACTTCCTCTCTCCATCCG-3′ | 4 | 8311.96 | 8316.4 |
| PMO-005 | 5′ -TCTCCATCCGAAAGAAGTATGAACC-3′ | 5 | 8421.09 | 8423.7 |
| PMO-006 | 5′ -TGGGATGGAAGAAAGTGCAGGGCAC-3′ | 6 | 8647.24 | 8649.7 |
| PMO-007 | 5′ -CAGCCAGAAATTTGGATCCATAGCC-3′ | 7 | 8437.09 | 8444.7 |
| PMO-008 | 5′ -CCCTGTGGGGAAACGAGGGTCAGCT-3′ | 8 | 8550.14 | 8552.5 |

MOE oligonucleotides were designed for screening. The designed oligonucleotides listed in Tables 12 and 2 were made by either Integrated DNA Technologies (www.idtdna.com) or GeneDesign (Ajinomoto Bio Pharma, https://ajibio-pharma.com/). Table 12 lists the top MOE sequences with their deconvoluted MS data. All MOE oligonucleotide listed in Tables 12 and 2 contain a hydroxyl at the 5′ end. All MOE oligonucleotides listed in Tables 12 and 2 contain 2′-O-MOE-modified ribonucleotides with phosphorothioate backbone except when noted. All MOE oligonucleotides listed in Tables 12 and 2 were synthesized with 5-methylcytosine 2′-O-MOE ribonucleotide. All MOE oligonucleotides listed in Tables 12 and 2 have stereorandom internucleotide linkages, and thus are called stereorandom MOE oligonucleotides. The general formula of the MOE oligonucleotides listed in Tables 12 and 2 depicted as free form is:

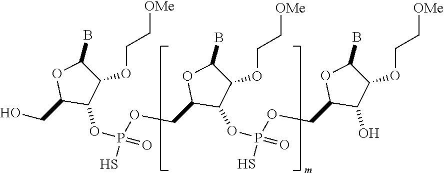

### General Formula for MOE Oligonucleotide Sequences

TABLE 12

| | | SEQ. | MW as | |
|---|---|---|---|---|
| | | ID | free | MS |
| ASO # | Seq. (5′ to 3′) | NO: | form | observed |
| MOE-009 | CACATGCACAGAGAGCTGGG | 9 | 8031.7 | 8033.80 |
| MOE-010 | GAGAGCTGGGGAGATTTGTA | 10 | 8077.1 | 8077.31 |
| MOE-011 | TCTCCATCCGAAAGAAGTAT | 11 | 7940.9 | 7942.85 |
| MOE-012 | ATCCGAAAGAAGTATGAACC | 12 | 7985.2 | 7986.62 |
| MOE-013 | ATGCTCAGGGAGCAGTTGTT | 13 | 8016.1 | 8016.17 |
| MOE-014 | GAGTCTCCTCCTGTACTTCT | 14 | 7894.4 | 7895.30 |
| MOE-015 | CGCACAAACCCTCCTGTACC | 15 | 7893.0 | 7893.84 |

### Example 3: Synthesis of PMO-302 (Stereopure Internucleotide Linkages (SD))

Synthesis of Stereopure PMO-302 Oligonucleotide with Unfunctionalized 5′-OH (CCTCACCTGTCACATGCACAGAGAG (SEQ ID NO: 2)).

Monomers used in the synthesis of PMO-302 are as follows: (reported in WO2017024264A2):

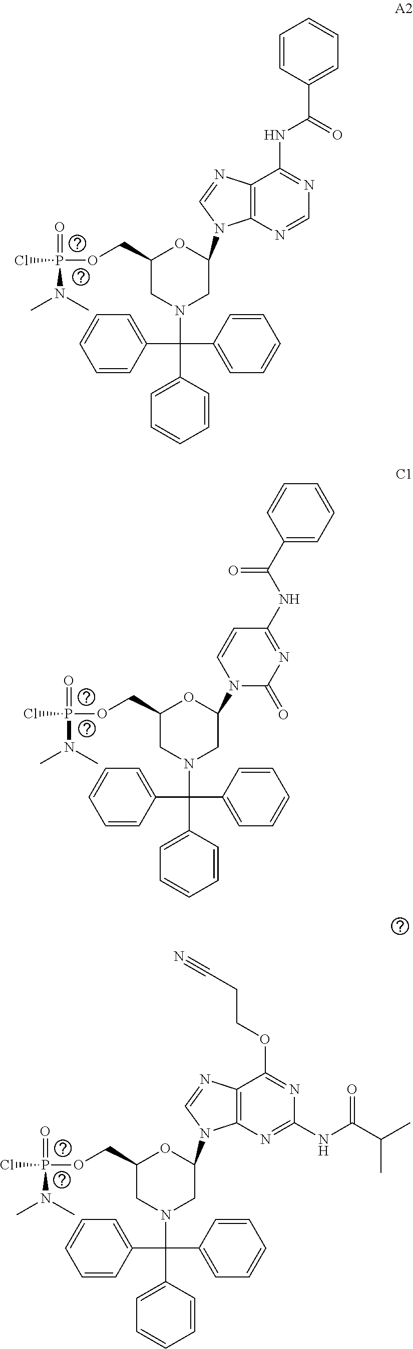

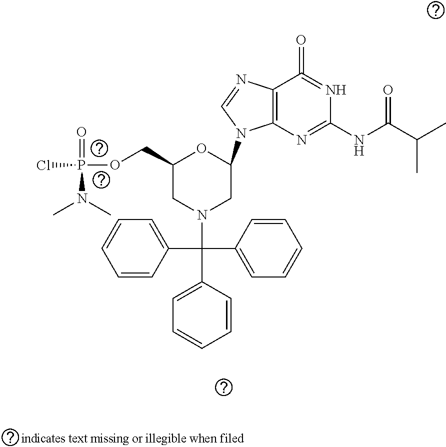

Synthesis of PMO-302 with 5-OH and Stereopure Internucleotide Linkages:
2-mer Synthesis:

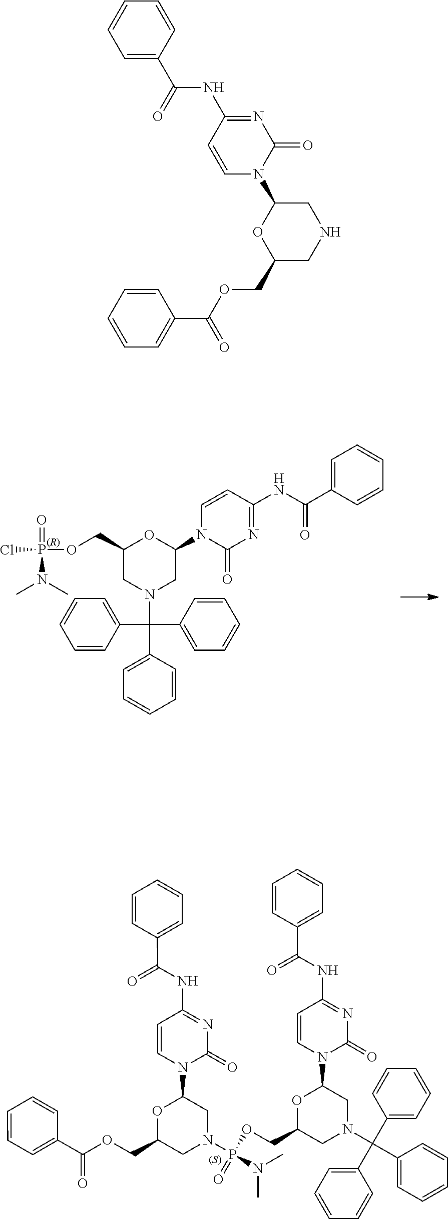

Unless otherwise noted all liquid ingredients were added via an appropriate size syringe. All reactions were conducted under N2 atmosphere. Filtrations and workup were done open to the air. Filtrations were conducted on a glass sintered funnel.

A flask with the amine ((2S,6R)-6-(4-benzamido-2-oxopyrimidin-1(2H)-yl)morpholin-2-yl)methyl benzoate (130 mg) was equipped with a stir bar and rubber septum. The atmosphere was exchanged with nitrogen and sparged. After 5 minutes, 1,3-dimethyl-2-imidazolidinone (2.2 mL) was added, followed by 1,2,2,6,6-pentamethylpiperidine (164 μL) via syringe at rt, and the mixture was allowed to form a solution. Solid ((2S,6R)-6-(4-benzamido-2-oxopyrimidin-1(2H)-yl)-4-tritylmorpholin-2-yl)methyl (R)-dimethylphosphoramidochloridate (219 mg) was added in one portion and the flask was sealed with the rubber septum. Stirring was continued for 3 h and the reaction monitored by UPLC MS. Upon completion, while stirring, MTBE (11.7 mL) was added over 1 minute. Precipitate was formed towards the end of the addition. n-Heptane (10 mL) was added. The oily mixture was allowed to settle for 10 minutes. While the heavy oil was settled, the cloudy supernatant was transferred by decantation to a 30 mL vial and centrifuged. This formed an additional oily residue on the bottom. The solvent was removed by decantation and the two oily residues were combined by dissolving into 1 mL of DCM and purified by flash chromatography with 0-5% MeOH in DCM. The fractions containing desired product were dried under vacuum to obtain ((2S,6R)-6-(4-benzamido-2-oxopyrimidin-1(2H)-yl)-4-((S)-(((2S,6R)-6-(4-benzamido-2-oxopyrimidin-1(2H)-yl)-4-tritylmorpholin-2-yl)methoxy)(dimethylamino)phosphoryl)morpholin-2-yl)methyl benzoate (270 mg) as a white foam. MS (ESI) m/z: [M+H]⁺ Calcd for C₆₀H₅₈N₉O₁₀P: 1096.40. Found: 1096.53.

Deprotection of 2-mer:

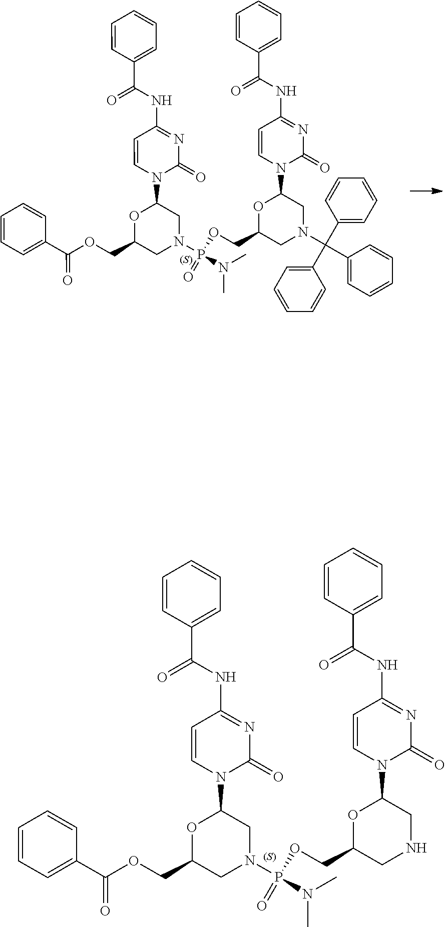

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-tritylmorpholin-2 yl)methoxy)(dimethylamino)phosphoryl)morpholin-2-yl)methyl benzoate (350 mg) was added DCM (3.5 mL). Ethanol (186 μL) was added via syringe at rt. TFA (160 μL) was added at rt dropwise over 30 seconds. The reaction mixture was stirred for 30 min and monitored by UPLC-MS. Upon completion, MTBE (14 mL) was added over 1 minute with a syringe. The suspension was stirred for 10 min and then sonicated. The suspension was filtered over a sintered filter funnel and rinsed with MTBE 10 mL (2×5 mL). The solids were dried, transferred to a new flask and then dissolved by addition of DCM (3.5 mL). 1,2,2,6,6-pentamethylpiperidine (292 μL) was added via syringe. After 10 min at rt, MTBE (15.8 mL) was added over 1 minute. White solids were formed. After 10 minutes the slurry was sonicated, filtered and rinsed with MTBE (e.g., 2×10 mL). It was then dried for 20 minutes under air flow and 1 hour under vacuum to obtain ((2S,6R)-6-(4-benzamido-2-oxopyrimidin-1(2H)-yl)-4-((S)-(((2S,6R)-6-(4-benzamido-2-oxopyrimidin-1(2H)-yl)morpholin-2-yl)methoxy)(dimethylamino)phosphoryl)morpholin-2-yl)methyl benzoate (243 mg). MS (ESI) m/z: [M+H]⁺ Calcd for C₄₁H₄₄N₉O₁₀P: 854.29. Found: 854.65.

3-mer Synthesis:

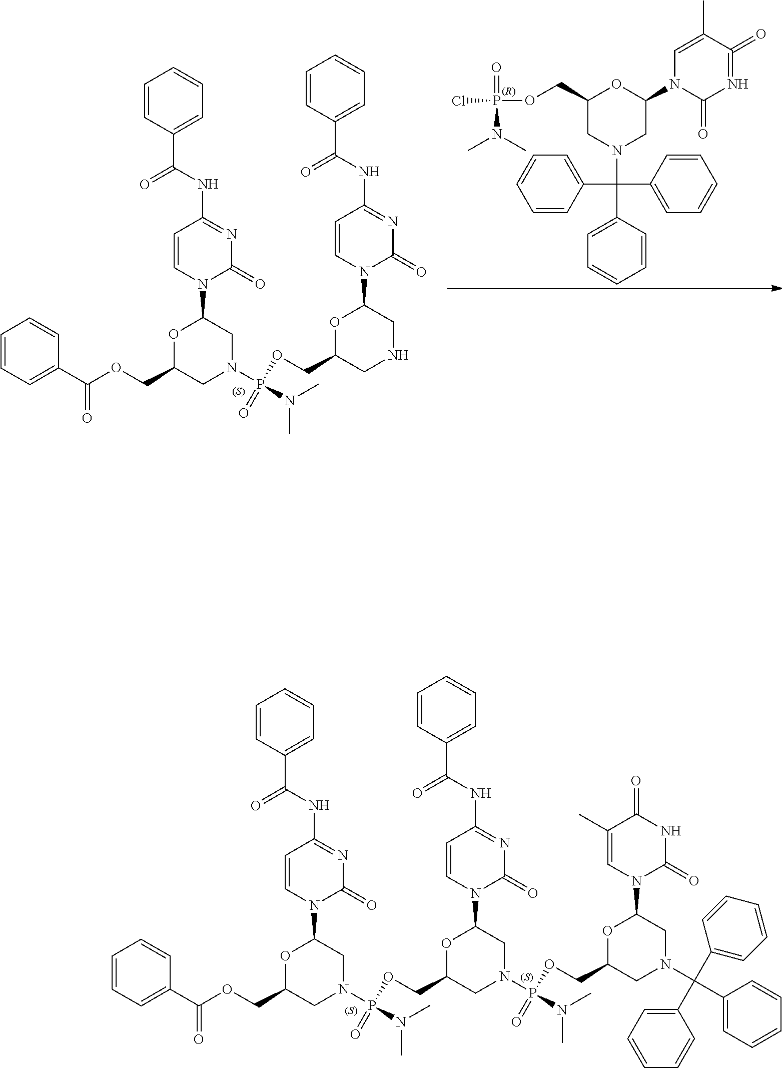

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)morpholin-2-yl)methoxy)(dimethylamino)phosphoryl)morpholin-2-yl)methyl benzoate (215 mg) were added 1,3-Dimethyl-2-imidazolidinone (2 mL) and 1,2,2,6,6-pentamethylpiperidine (138 μL) under nitrogen. After 2-min ((2S,6R)-6-(5-methyl-2,4-dioxo-3,4-dihydropyrimidin-1(2H)-yl)-4-tritylmorpholin-2-yl)methyl-(R)-dimethylphosphoramidochloridate (169 mg) was added, and the reaction was stirred for 3 h at rt. Upon completion, ethyl acetate (2.6 mL) was added and then MTBE (14 mL) was added. The resulting white precipitate was filtered, rinsed with MTBE (2×5 mL) and dried under vacuum to obtain ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(dimethylamino)(((2S,6R)-6-(5-methyl-2,4-dioxo-3,4-dihydropyrimidin-1(2H)-yl)-4-tritylmorpholin-2-yl)methoxy)phosphoryl)morpholin-2-yl)methoxy)(dimethylamino)phosphoryl)morpholin-2-yl)methyl benzoate (350 mg). MS (ESI) m/z: [M+H]⁺ Calcd for C₇₂H₇₇N₁₃O₁₅P₂: 1426.51. Found: 1427.74.

Deprotection of 3-mer:

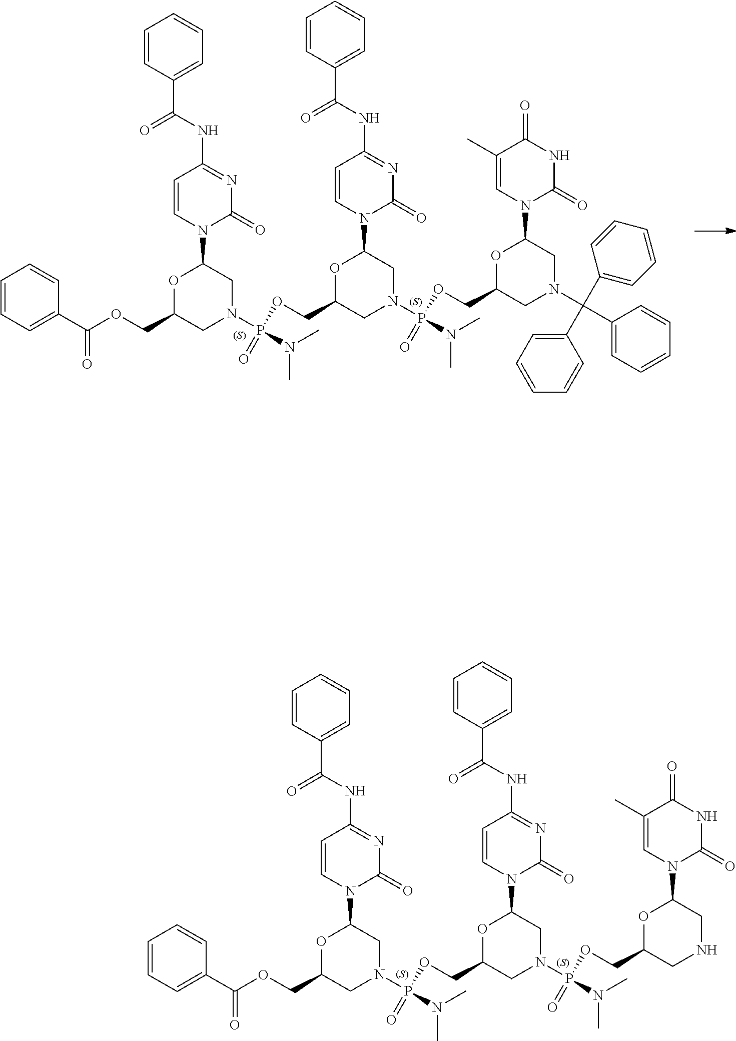

To a flask was added ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(dimethylamino)(((2S,6R)-6-(5-methyl-2,4-dioxo-3,4-dihydropyrimidin-1(2H)-yl)-4-tritylmorpholin-2-yl)methoxy)phosphoryl)morpholin-2-yl)methoxy)(dimethylamino)phosphoryl)morpholin-2-yl)methyl benzoate (350 mg) and DCM (4.2 mL). Ethanol (143 μL) and then TFA (95 μL) was slowly at rt added. The reaction mixture was stirred for 2 hours at rt. Upon completion, MTBE (15 mL) was added. The solids were filtered and rinsed with MTBE (10 mL). The solids were dried and then transferred to a flask and dissolved by addition of DCM (2.7 mL). 1,2,2,6,6-pentamethylpiperidine (224 μL) was added at rt and the reaction mixture was stirred at rt for 10 minutes. MTBE (15 mL) was added, and the resulting slurry was stirred for 10 minutes and sonicated, filtered, and rinsed with MTBE (10 mL). The trimer was obtained as free base (297 mg). MS (ESI) m/z: [M+H]⁺ Calcd for C₅₃H₆₃N₁₃O₁₅P₂: 1184.40. Found: 1185.

4-mer Synthesis:

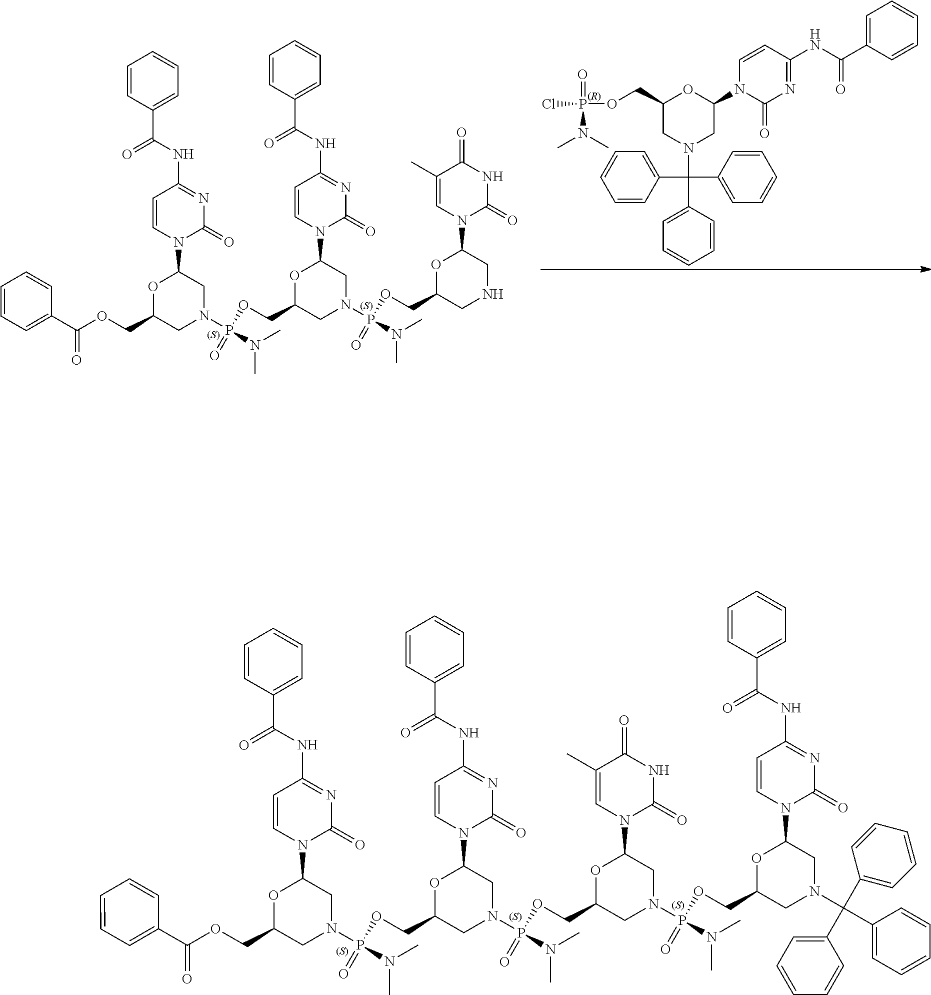

To a flask was added ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(dimethylamino)(((2S,6R)-6-(5-methyl-2,4-dioxo-3,4-dihydropyrimidin-1(2H)-yl)morpholin-2-yl)methoxy)phosphoryl)morpholin-2-yl)methoxy)(dimethylamino)phosphoryl)morpholin-2-yl)methyl benzoate (292 mg) and the flask was purged with nitrogen. Added 1,3-Dimethyl-2-imidazolidinone (2.9 mL) and then 1,2,2,6,6-pentamethylpiperidine (135 μL). ((2S,6R)-6-(4-Benzamido-2-oxopyrimidin-1(2H)-yl)-4-tritylmorpholin-2-yl)methyl (R)-dimethylphosphoramidochloridate (207 mg) was added in one portion and the reaction was stirred at rt for at least 1 h monitoring for completion by HPLC-MS. Ethyl acetate (2.9 mL) was charged followed by MTBE (14 mL). The slurry was stirred for 15 minutes and filtered, washed with MTBE 2×5 mL. The resulting solids were dried under vacuum for 10 minutes and then collected to a new flask, dried under vacuum to afford 430 mg of 4-mer. MS (ESI) m/z: [M+H]⁺ Calcd for C₉₀H₉₉N₁₈O₂₀P₃: 1846.65. Found: 1847.

Deprotection of 4-mer:

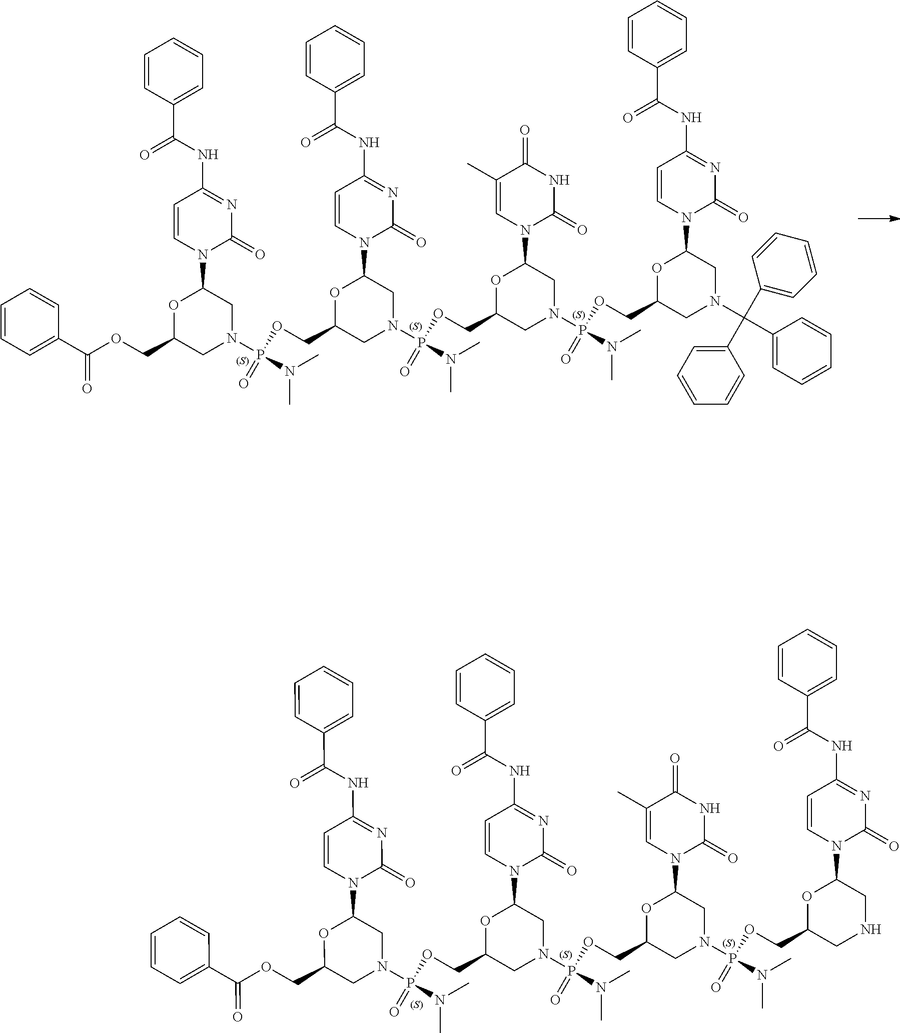

To a flask was added ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-tritylmorpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (430 mg). Added DCM (4.3 mL) and ethanol (272 μL). After a solution was formed, added TFA (135 μL). The reaction mixture was stirred for 2.5 h when it was deemed completed by HPLC analysis. Added ethyl acetate (3.0 mL) and MTBE (10.8 mL) over 1 minute. Solid precipitate were formed during MTBE addition. Upon completed MTBE addition, the solids were stirred for 10 minutes and sonicated three times. Filtered and rinsed with MTBE 2×5 mL. The solids were dried and then dissolved in DCM (4.3 mL) and treated with 1,2,2,6,6-pentamethylpiperidine (319 μL). After 5 min, the desired product was precipitated by addition of ethyl acetate (3.0 mL) and MTBE (10.8 mL) over 1 minute. The solids were filtered, rinsed with MTBE and dried under vacuum overnight to afford ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (330 mg). MS (ESI) m/z: [M+H]⁺ Calcd for C₇₁H₈₅N₁₈O₂₀P₃: 1603.54. Found: 1605.

5-mer Synthesis:

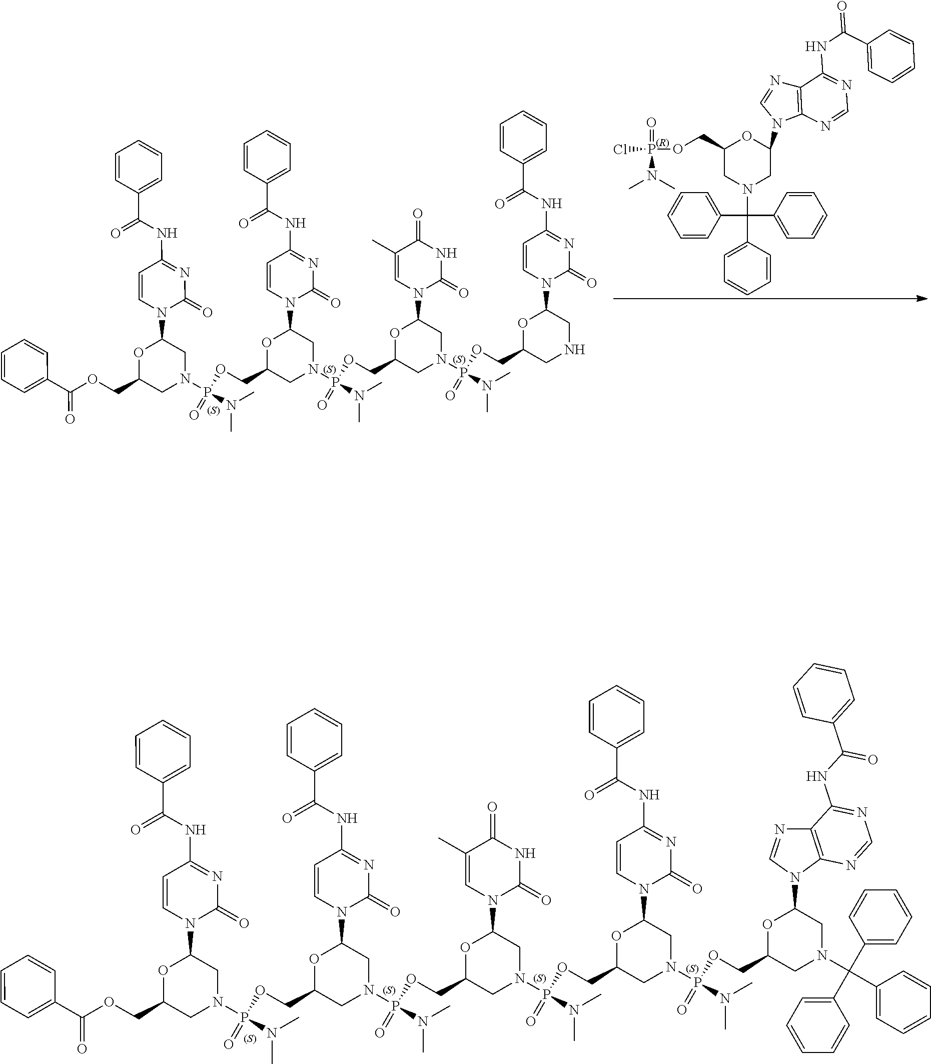

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (330 mg) was added 1,3-Dimethyl-2-imidazolidinone (3.3 mL) and 1,2,2,6,6-pentamethylpiperidine (113 μL). After the residue fully dissolved, added ((2S,6R)-6-(6-benzamido-9H-purin-9-yl)-4-tritylmorpholin-2-yl)methyl (R)-dimethylphosphoramidochloridate (178 mg) at rt. The reaction mixture was stirred at rt for 5 h and then added ethyl acetate (6.6 mL) and MTBE (13.2 mL). The white precipitate was filtered and dried. The solid was dissolved in DCM 2 mL and purified by automated silica gel chromatography on a 25 g cartridge with 0-20% MeOH in DCM. Afforded 345 mg of desired product 5-mer. MS (ESI) m/z: Calcd for C₁₀₉H₁₂₁N₂₅O₂₄P₄: [(M+2H)/2]⁺ 1145.4. Found: 1145.6.

Deprotection of 5-mer:

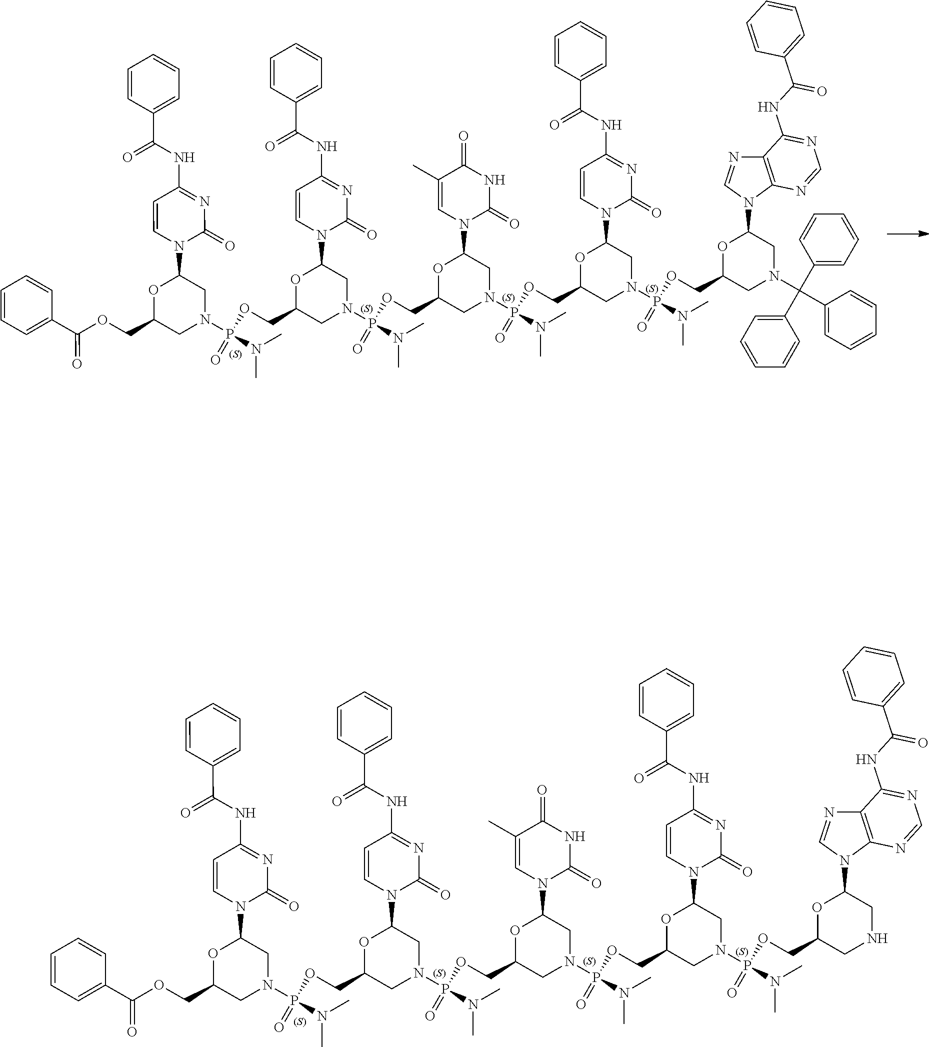

To a flask was added ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-6-(6-benzamido-9H-purin-9-yl)-4-tritylmorpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (328 mg). Added DCM (3.1 mL) and then ethanol (167 μL). Added TFA (66.2 μL) at rt and stirred at rt for 3h. Added additional 3 drops (ca 15 μL) of TFA. The reaction was monitored by HPLC-MS and upon completion (disappearance of starting material peak), added ethyl acetate (11.8 mL) followed by stirring for 5 min. Filtered and rinsed with EtOAc (2 mL) and MTBE (5 mL). Additional solids were formed in the mother liquor which were also harvested by second filtration. The combined solids were placed into a reaction flask. Added DCM (2.3 mL) and 1,2,2,6,6-pentamethylpiperidine (209 μL). Stirred for 15 min then added EtOAc (2.6 mL) and MTBE (10.5 mL). The resulting solids were filtered and rinsed by MTBE 2×3 mL then dried under vacuum, collected to afford 280 mg deprotected 5-mer. MS (ESI) m/z: Calcd for C₉₀H₁₀₇N₂₅O₂₄P₄: [(M+2H)/2]⁺ 1023.8. Found: 1024.12.

6-mer synthesis:

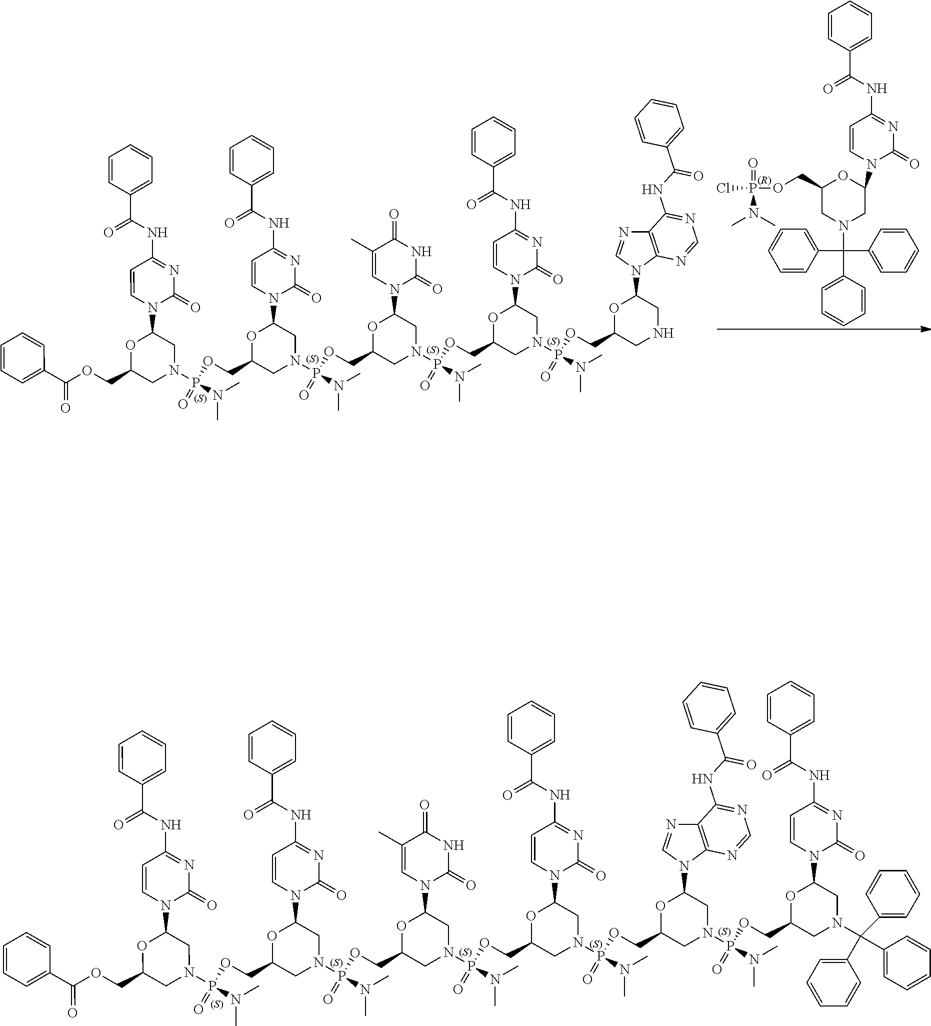

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (265 mg) was added 1,3-Dimethyl-2-imidazolidinone (2.7 mL). Added 1,2,2,6,6-pentamethylpiperidine (71.0 μL). Added ((2S,6R)-6-(4-benzamido-2-oxopyrimidin-1(2H)-yl)-4-tritylmorpholin-2-yl)methyl (R)-dimethylphosphoramidochloridate (108 mg) at rt as solid. After 3 hours at rt, upon completion as judged by HPLC analysis, added EtOAc (5.3 mL) and MTBE (10.6 mL) over 2-3 min each. Filtered and rinsed with MTBE 2×3 mL. After drying with air flow for 2-3 min the solids turned to sticky mass. The solids transferred to same flask with 10 mL DCM and concentrated under vacuum. Isolated ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-tritylmorpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (354 mg). MS (ESI) m/z: Calcd for C₁₂₇H₁₄₃N₃₀O₂₉P₅: [M+2H/2]⁺ 1354.97. Found: 1354.73.

Deprotection of 6-mer:

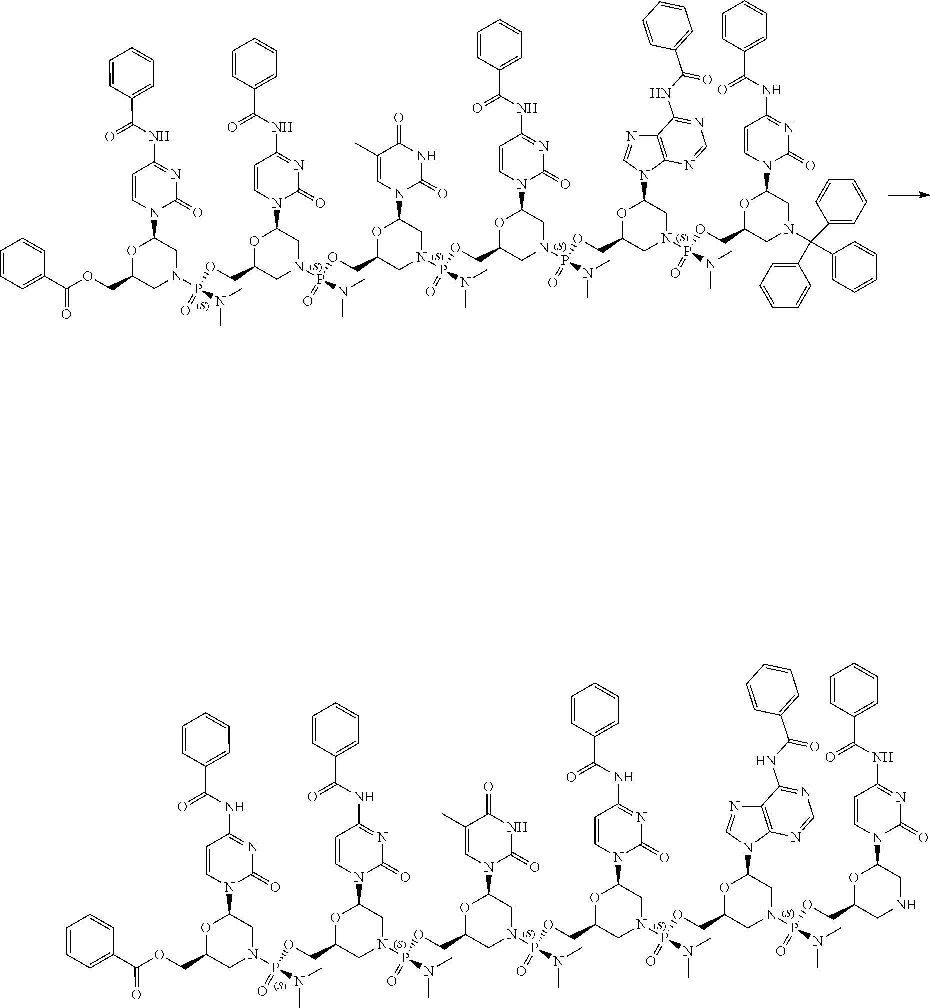

To a flask with the dried evaporated solids from previous step (6-mer) was added DCM (3.2 mL) and ethanol (155 μL). After the solids dissolved completely, added TFA (71.7 μL). The mixture was stirred for 2 h and the reaction was not completed according to HPLC analysis. Added additional 50 μL TFA and continued to stir for additional 6h. Added EtOAc (2.9 mL) then MTBE (11 mL). The resulting solids were filtered and rinsed with 4:1 MTBE/EtOAc (12 mL). Isolated ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (295 mg). MS (ESI) m/z: Calcd for C₁₀₈H₁₂₉N₃₀O₂₉P₅: [(M+2H)/2]⁺ 1233.92. Found: 1233.68.

Synthesis of 7-mer:

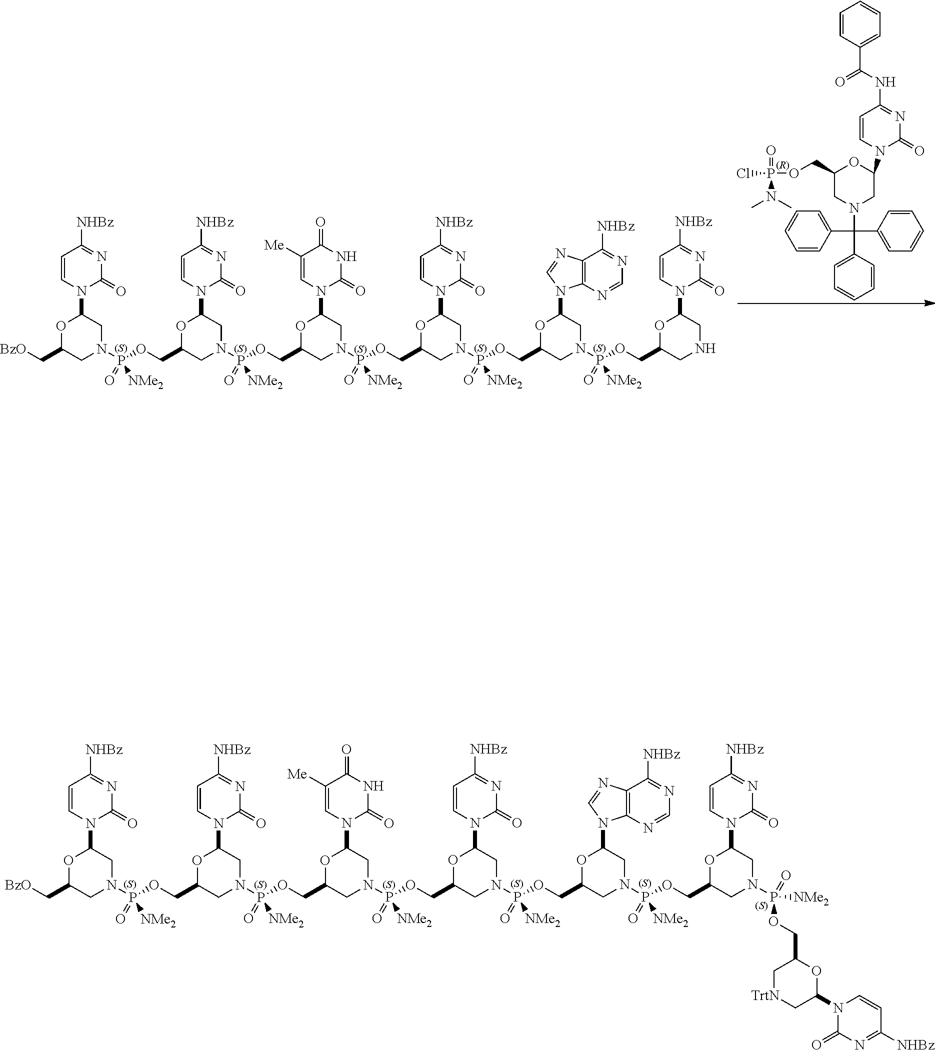

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (295 mg) was added 1,3-Dimethyl-2-imidazolidinone (2.9 mL) and then 1,2,2,6,6-pentamethylpiperidine (65.6 μL) at rt. ((2S,6R)-6-(4-benzamido-2-oxopyrimidin-1 (2H)-yl)-4-tritylmorpholin-2-yl)methyl (R)-dimethylphosphoramidochloridate was added at rt as a solid (100 mg). The reaction was stirred for 3 h at rt. Upon completion by HPLC analysis, added EtOAc (5.9 mL) and MTBE (11.8 mL) over 2-3 min each. The solids were filtered and rinsed with MTBE 2×5 mL. After drying with air flow for 2-3 min the solids were transferred to a flask and dried under vacuum for 1 h to afford 7-mer (430 mg). MS (ESI) m/z: Calcd for C₁₄₅H₁₆₅N₃₅O₃₄P₆: [(M+2H)/2]⁺ 1564.85. Found: 1564.77.

Deprotection of 7-mer:

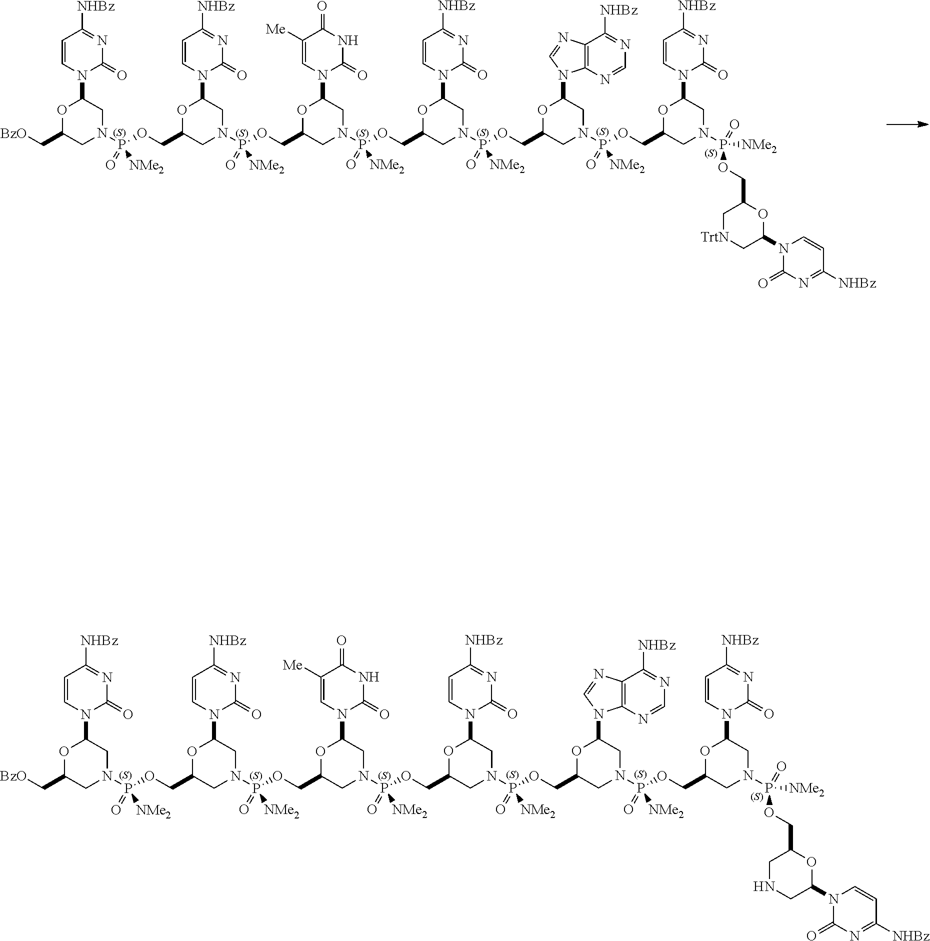

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-6-(4-benzamido-2-oxopyrimidin-1(2H)-yl)-4-tritylmorpholin-2-yl)methoxy)(dimethylamino)phosphoryl)morpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (374 mg) was added DCM (3.0 mL) and ethanol (140 μL). Added TFA (138 μL) dropwise at rt over 30 sec. After 30 minutes, added EtOAc (7.5 mL) and added MTBE (7.5 mL). Filtered and rinsed with MTBE 2×3 mL. The solids were dried in the filter funnel under air flow and then transferred to a flask. Dissolved in DCM (3.9 mL) and EtOH (140 μL), and added 1,2,2,6,6-pentamethylpiperidine (109 μL). After 10 minutes, the solution was treated with added EtOAc (7.5 mL) and MTBE (7.5 mL). The resulting solids were filtered and rinsed with MTBE 2×3 mL. The solids were dried in funnel and then transferred to a flask, dried under vacuum to obtain total ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-6-(4-benzamido-2-oxopyrimidin-1(2H)-yl)morpholin-2-yl)methoxy)(dimethylamino)phosphoryl)morpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (345 mg). MS (ESI) m/z: Calcd for C₁₂₆H₁₅₁N₃₅O₃₄P: [(M+2H)/2]⁺ 1443.48. Found: 1444.

From 8-mer to 25-mer, general procedures were used for coupling, deprotection and free basing:

General procedure A for coupling: To a flask with dried PMO oligonucleotide (free base PMO oligonucleotide) (1 wt, 1 equiv.) was added 1,3-dimethyl-2-imidazolidinone (6-10 volumes compared to free base PMO oligonucleotide) and then 1,2,2,6,6-pentamethylpiperidine (3-5 equiv.). The mixture was stirred and sonicated until all solids dissolved. Activated monomer (R)-dimethylphosphoramidochloridate (1.3-2.5 equiv.) was added as a solid in a single portion under N2 atmosphere. The reaction mixture was stirred for a minimum of 3h (18-24 h at stages 15-25mer) and monitored for completion by UPLC MS (>99.5% target by UV or no detectable starting material mass). Additional (R)-dimethylphosphoramidochloridate was optionally added if target conversion criteria was not reached. Upon completion, the reaction mixture was charged with EtOAc (10-40 vols) and MTBE (10-40 volumes as compared to free base PMO oligonucleotide) to form a white precipitate. The solids were filtered on a sintered funnel, rinsed with EtOAc/MTBE 1:1, dried under vacuum and collected to afford “trityl-protected PMO oligonucleotide” for the next step. General yield was 90-100%.

### General Procedure B for Trityl Deprotection and Free Basing:

Trityl deblock solution was prepared as follows: To a flask were added DCM (8 mL), 2,2,2-trifluoroethanol (2 mL), 4-cyanopyridine (100 mg), ethanol (100 μL) and trifluoroacetic acid (105 mg) in that order. The solution was mixed until all components are dissolved and then used in deprotection as is.

Step 1—trityl deprotection: To a flask with “trityl-protected PMO oligonucleotide” (1 wt, 1 equiv.) was added trityl deblock solution (8 volumes compared to trityl-protected PMO oligonucleotide mass). The reaction mixture was stirred for 5-30 minutes and monitored by UPLC MS. Upon completion (>99.5% target), added EtOAc (10-40 vols) and MTBE (10-40 volumes) to form a white precipitate. The solids were filtered on a sintered funnel, rinsed with EtOAc/MTBE 1:1, dried under vacuum and collected to afford “TFA salt PMO oligonucleotide” for the next step.

Step 2—free basing: To a flask with “TFA salt PMO oligonucleotide” (1 wt, 1 equiv.) was added DCM (7-10 vols compared to TFA salt PMO oligonucleotide mass) and EtOH (0.3-0.5 vol). The solution was treated with 1,2,2,6,6-pentamethylpiperidine (5 equiv.). The reaction mixture was stirred for 5-10 minutes and then treated with EtOAc (10-40 vols) and MTBE (10-40 volumes) to form a white precipitate. The solids were rinsed with EtOAc/MTBE 1:1, dried under vacuum and collected for the next step.

Coupling to 8-mer:

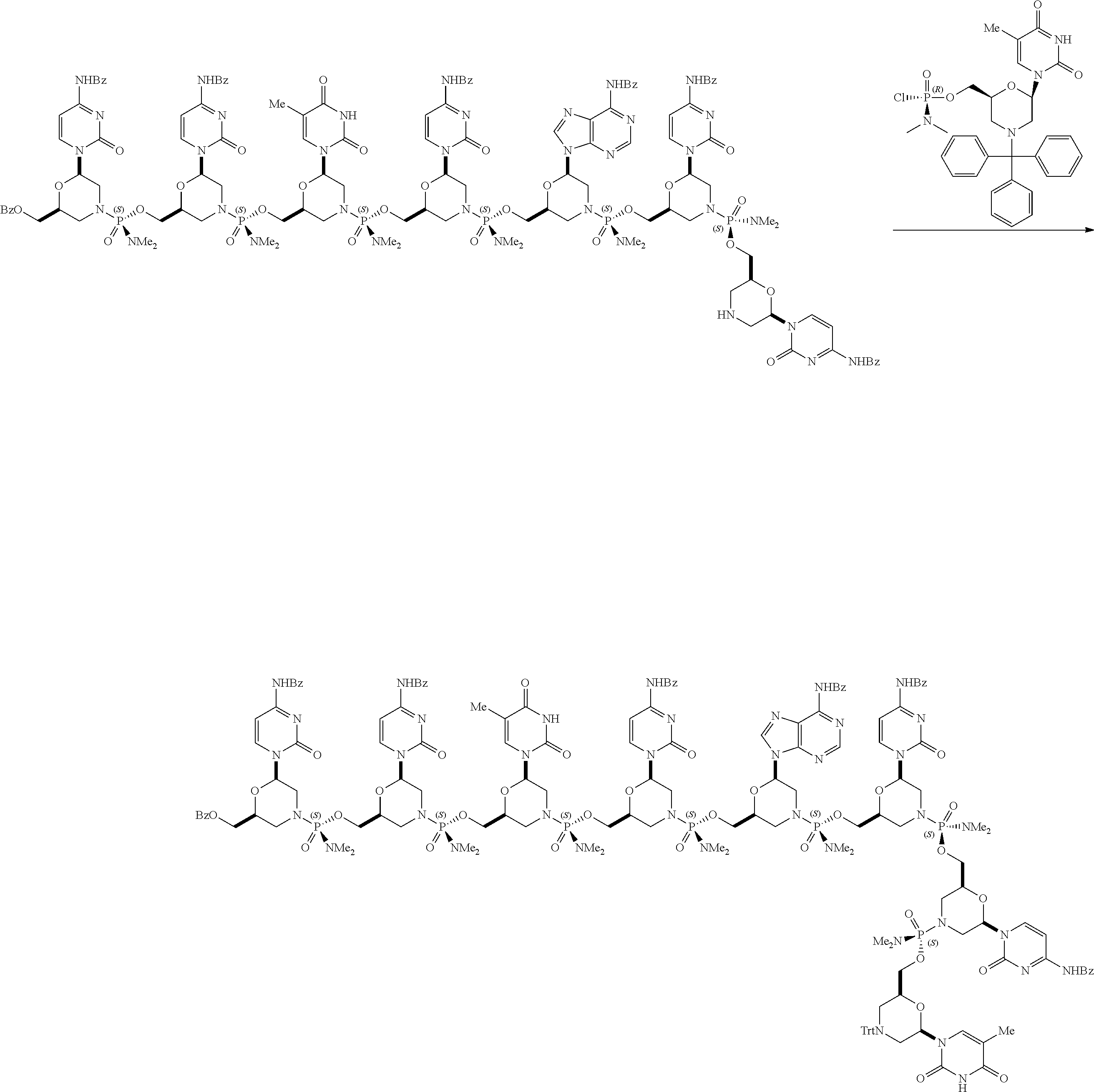

Using General Procedure A: reaction of 7-mer (340 mg) with ((2S,6R)-6-(5-methyl-2,4-dioxo-3,4-dihydropyrimidin-1(2H)-yl)-4-tritylmorpholin-2-yl)methyl (R)-dimethylphosphoramidochloridate (86 mg) afforded 8-mer (403 mg). MS (ESI) m/z: Calcd for C₁₅₇H₁₈₄N₃₉O₃₉P₇: [M+2H/2]⁺ 1730.09. Found: 1730.

Deprotection of 8-mer:

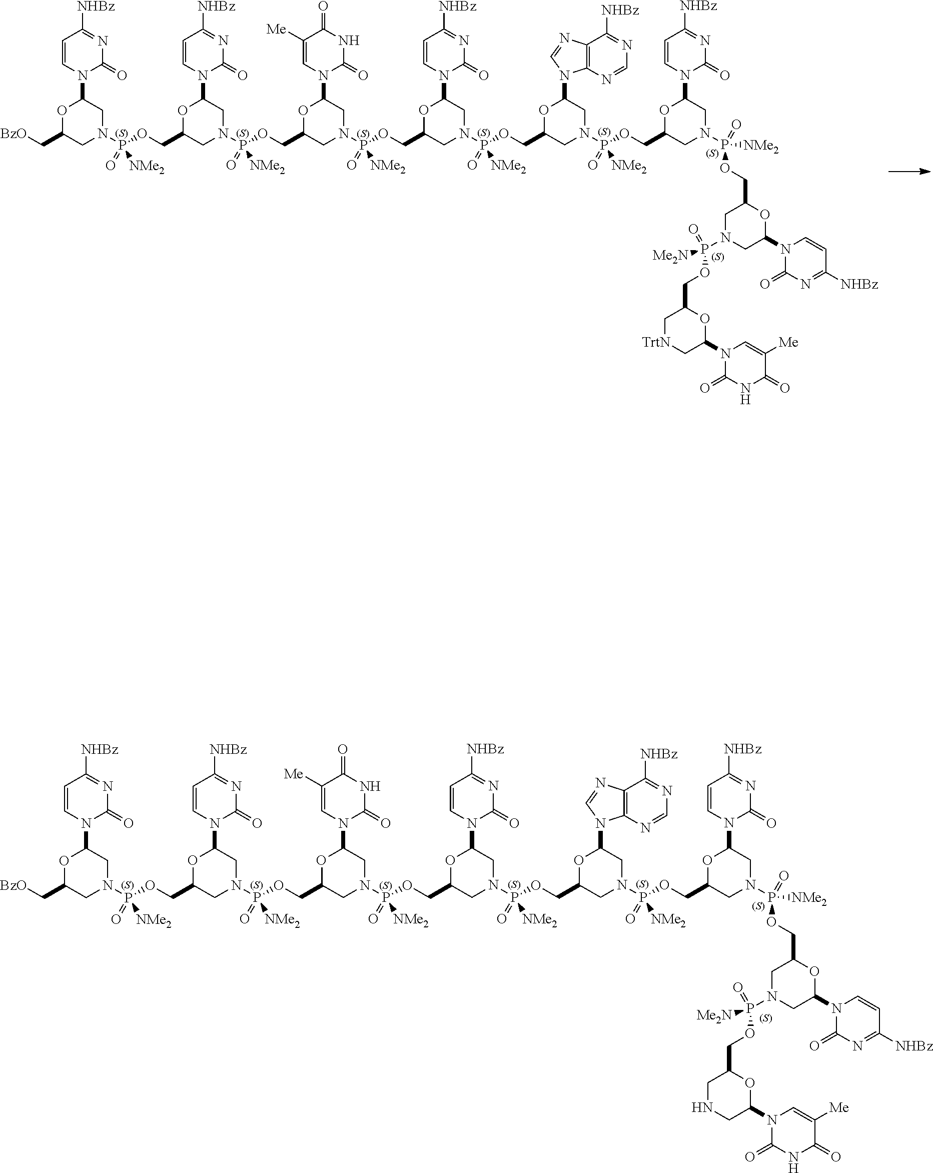

Using General Procedure B: reaction of trityl protected 8-mer (380 mg) afforded free base 8-mer (353 mg). MS (ESI) m/z: Calcd for C₁₃₈H₁₇₀N₃₉O₃P₇: [M+2H/2]⁺ 1608.53. Found: 1609.

Coupling to 9-mer:

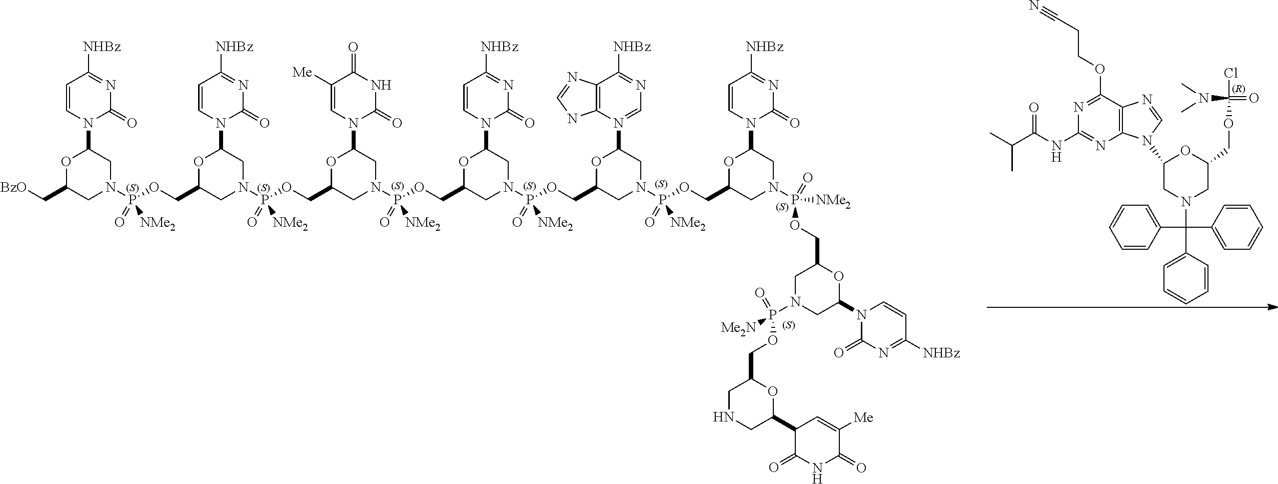

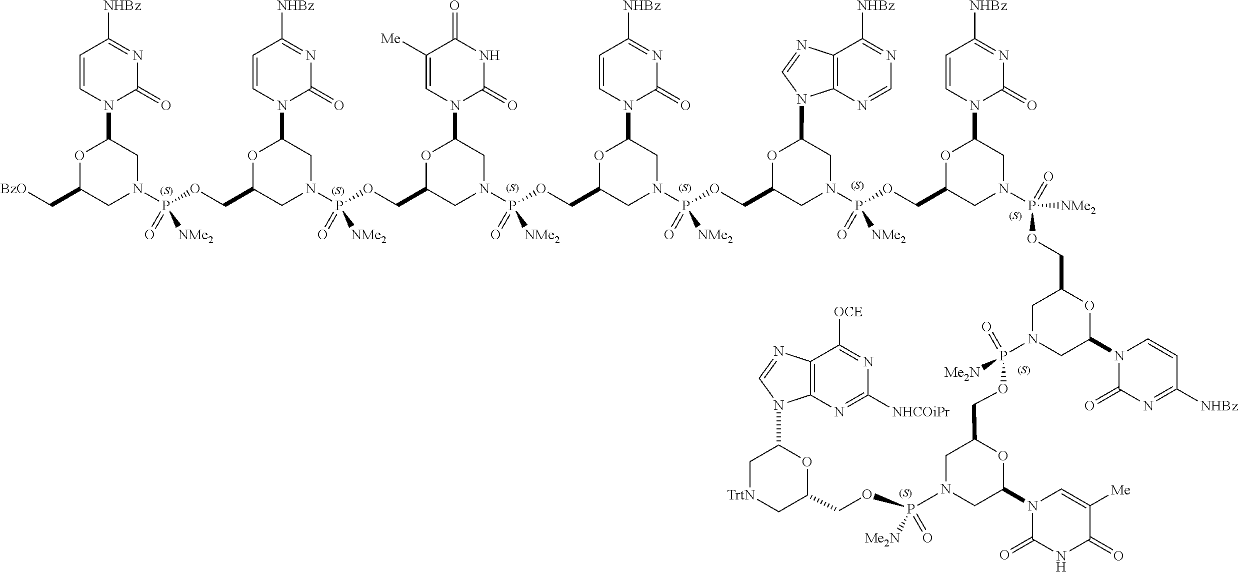

Using General Procedure A: reaction of 8-mer (370 mg) with ((2S,6R)-6-(6-(2-cyanoethoxy)-2-isobutyramido-9H-purin-9-yl)-4-tritylmorpholin-2-yl)methyl (R)-dimethylphosphoramidochloridate (105 mg) afforded 9-mer (453 mg).

Deprotection of 9-mer:

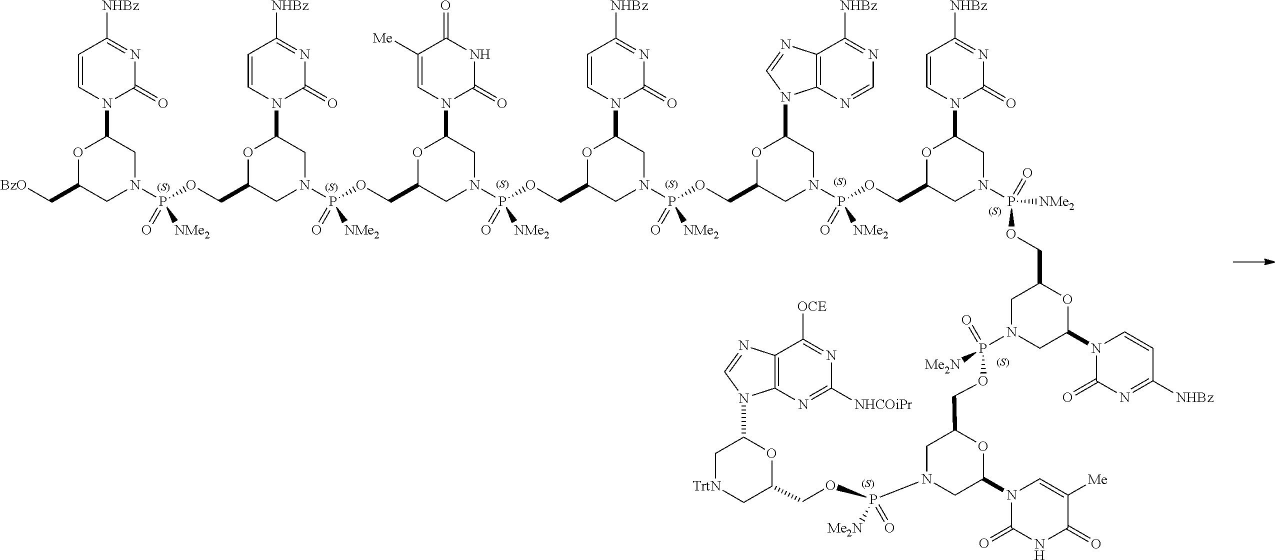

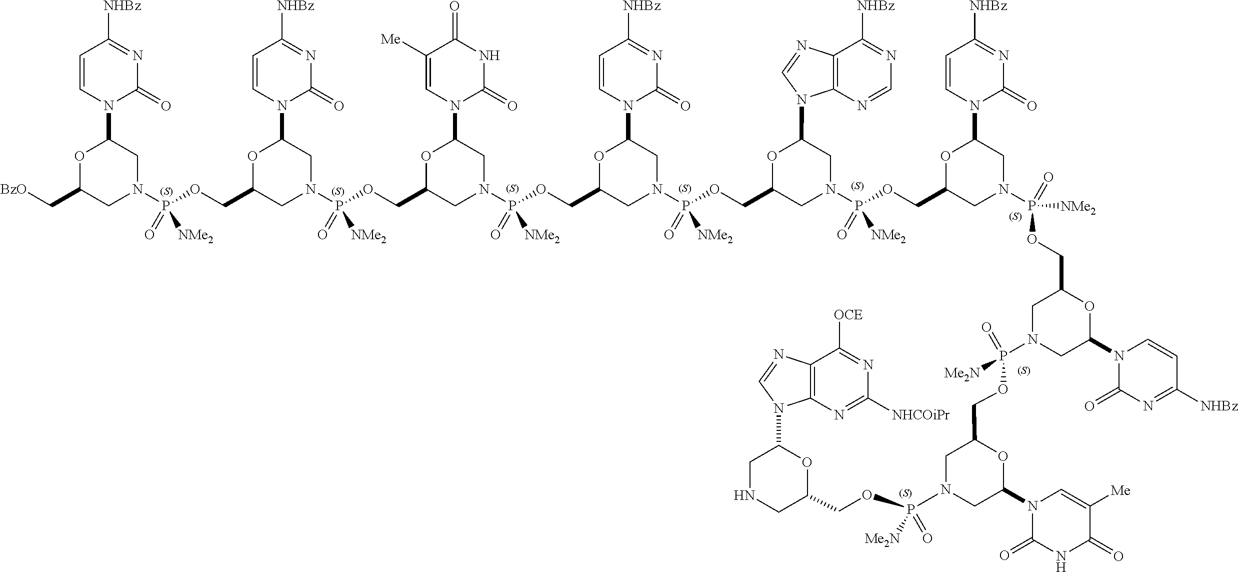

Using General Procedure B: reaction of trityl protected 9-mer (453 mg) afforded free base 9-mer (411 mg).

Coupling to 10-mer:

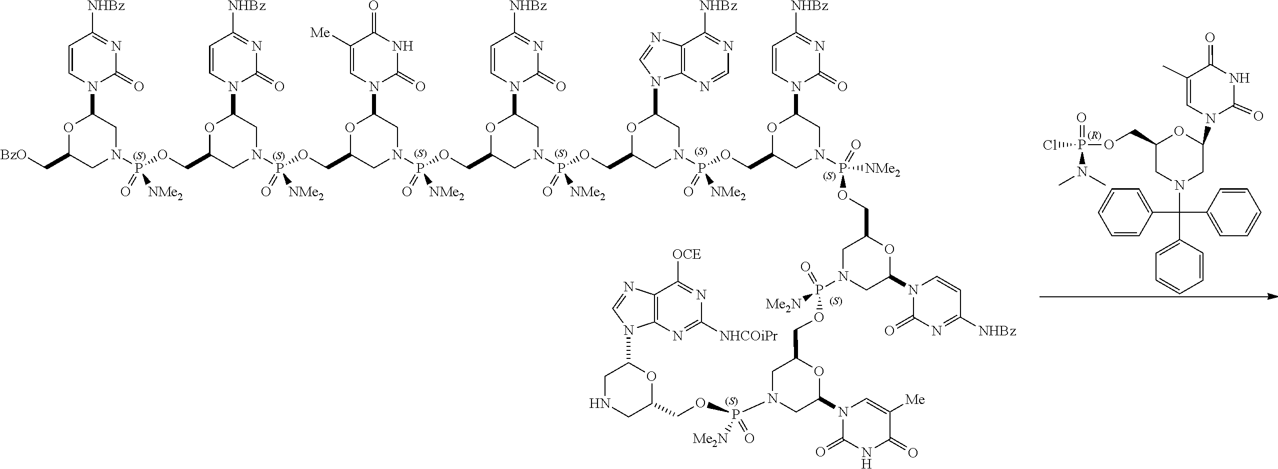

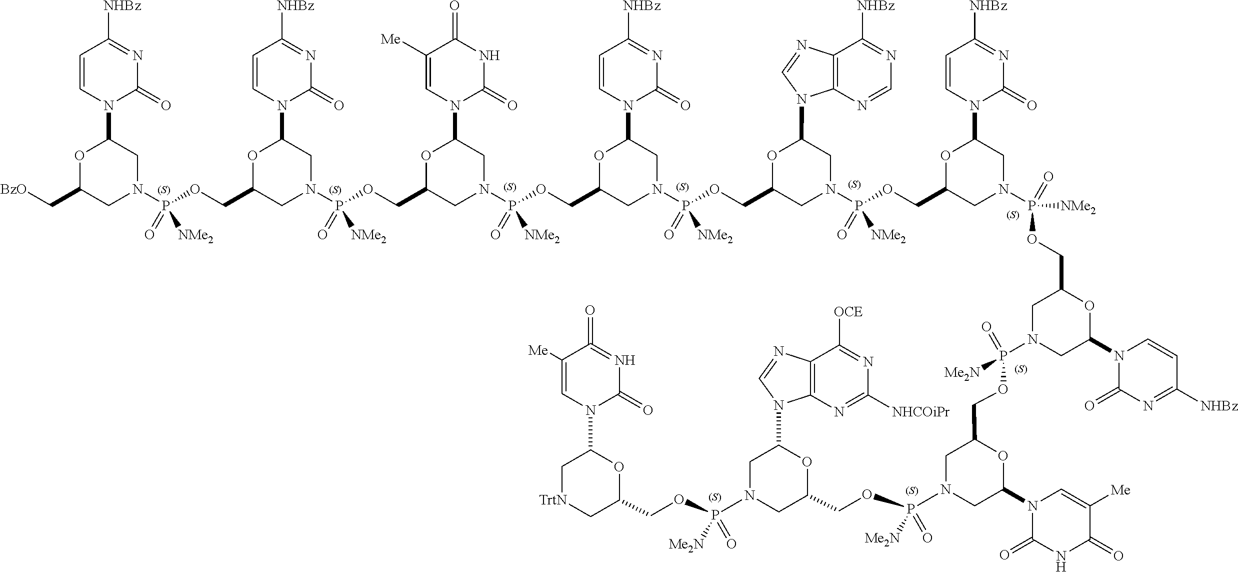

Using General Procedure A: reaction of 9-mer (405 mg) with ((2S,6R)-6-(5-methyl-2,4-dioxo-3,4-dihydropyrimidin-1(2H)-yl)-4-tritylmorpholin-2-yl)methyl (R)-dimethylphosphoramidochloridate (80 mg) afforded 10-mer (469 mg). MS (ESI) m/z: Calcd for C₁₈₈H₂₃₀N₅₁O₄₉P₉: [M+3H/3]⁺ 1422.8. Found: 1423.3.

Deprotection of 10-mer:

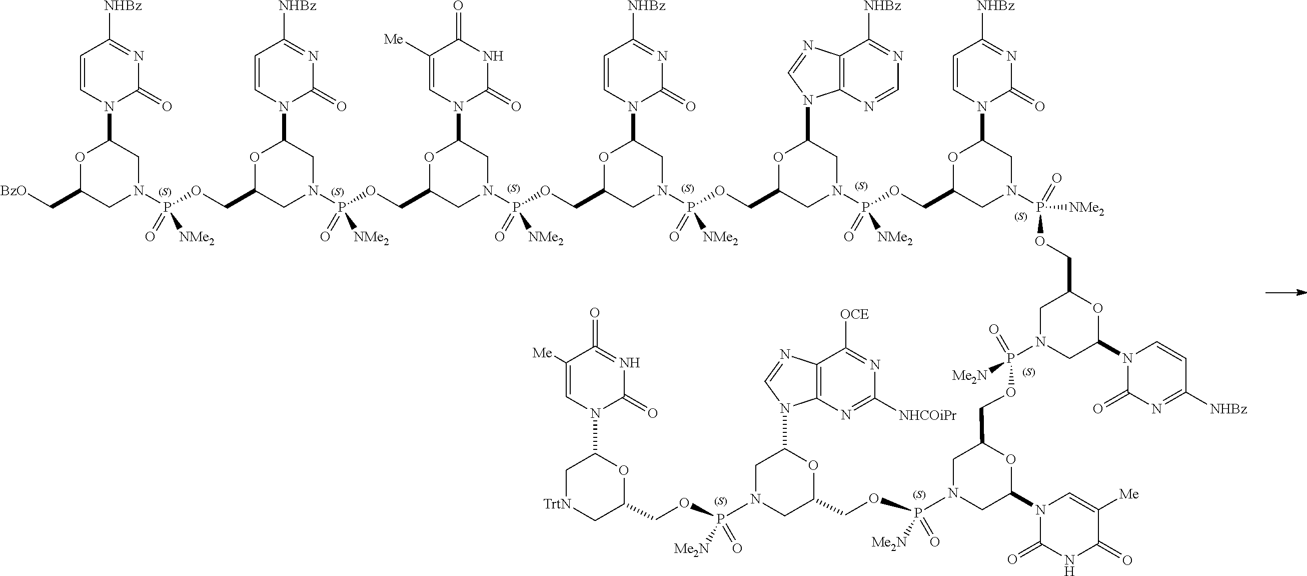

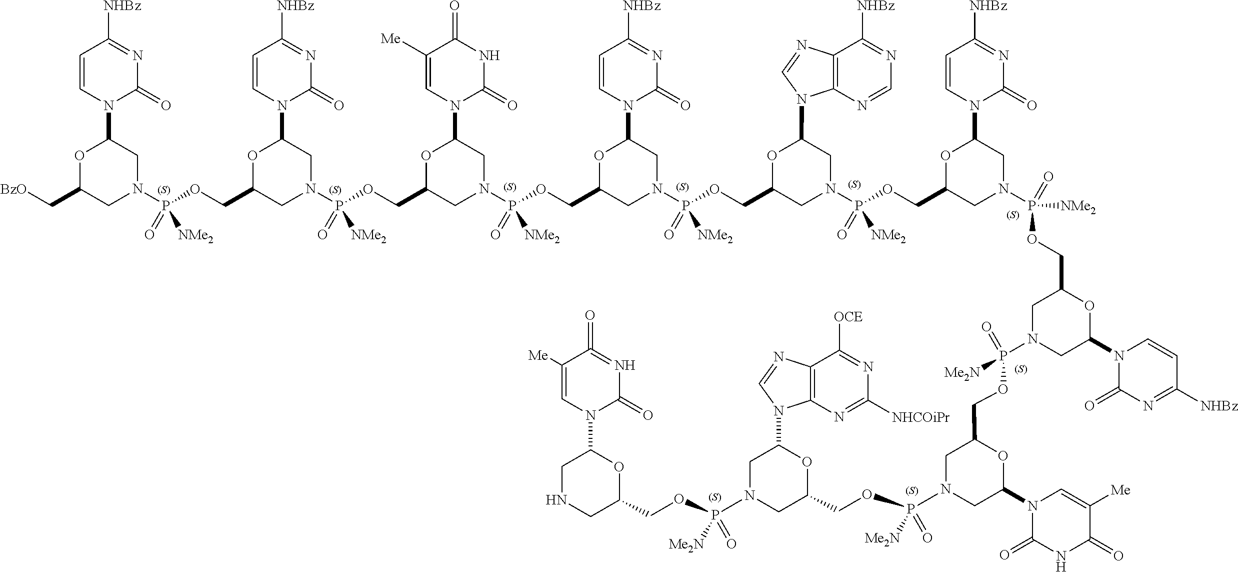

Using General Procedure B: reaction of trityl protected 10-mer (450 mg) afforded free base 10-mer (435 mg).

Coupling to 11-mer:

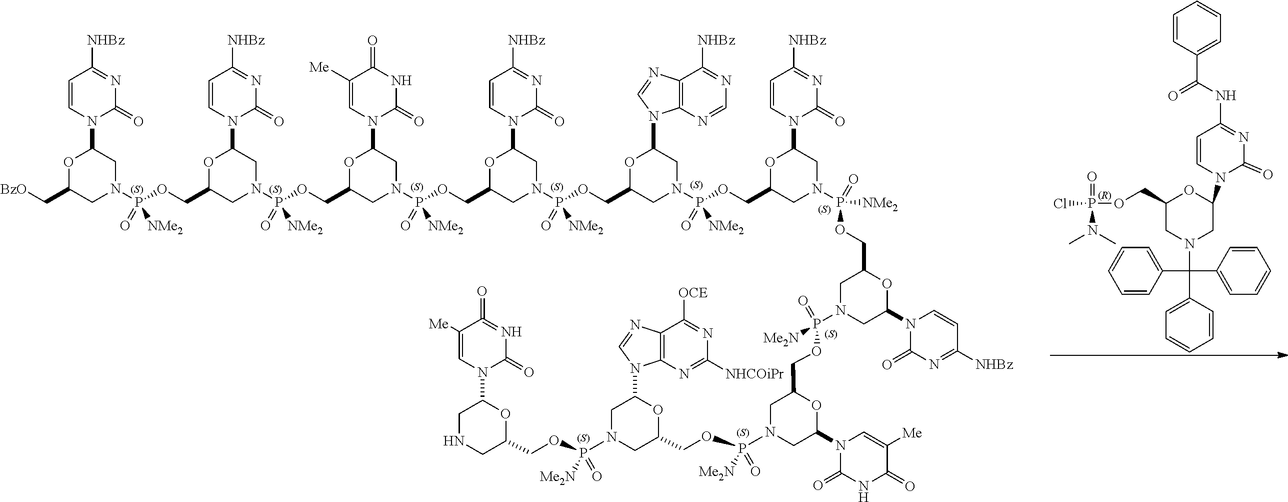

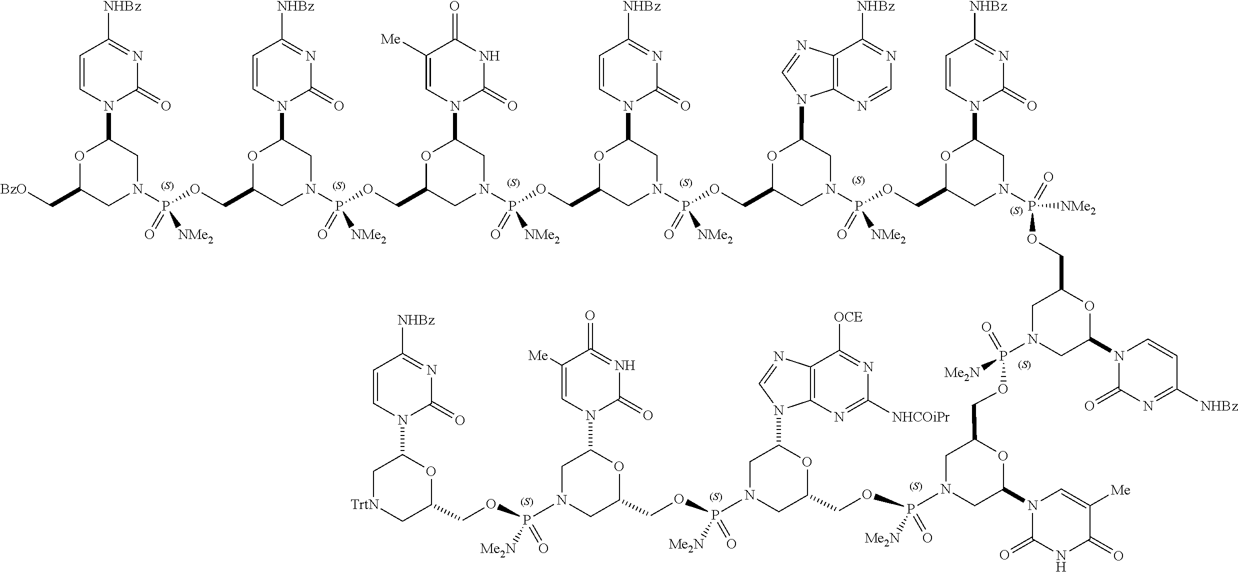

Using General Procedure A: reaction of 10-mer (435 mg) with ((2S,6R)-6-(4-benzamido-2-oxopyrimidin-1(2H)-yl)-4-tritylmorpholin-2-yl)methyl (R)-dimethylphosphoramidochloridate (98 mg) afforded 11-mer (512 mg).

Deprotection of 11-mer:

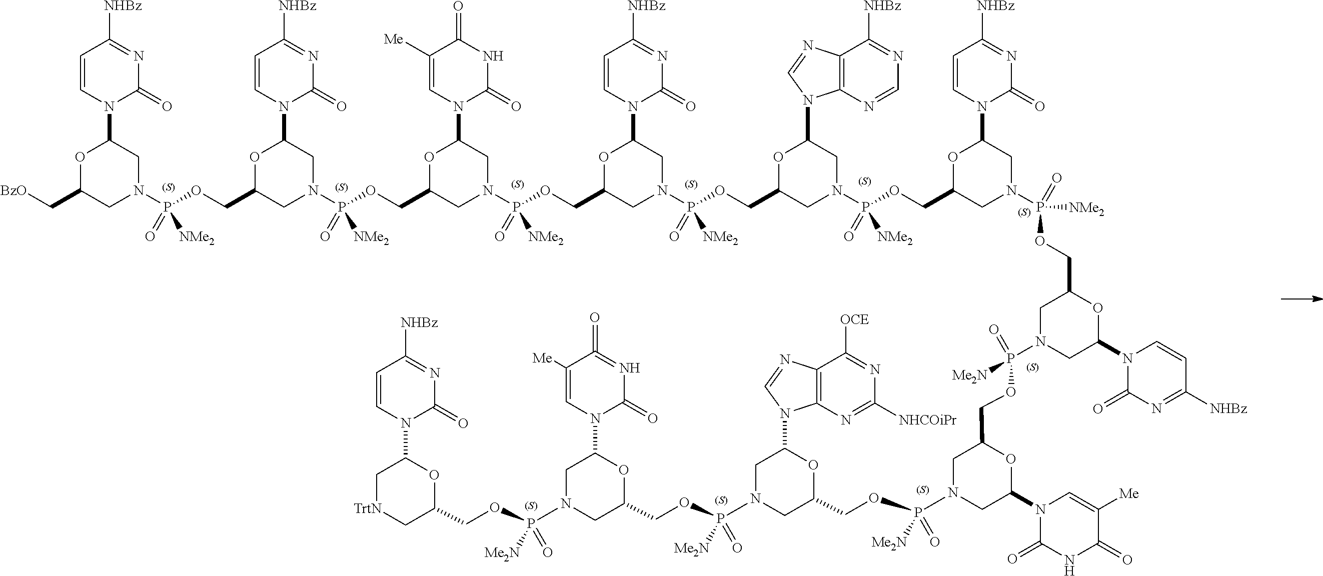

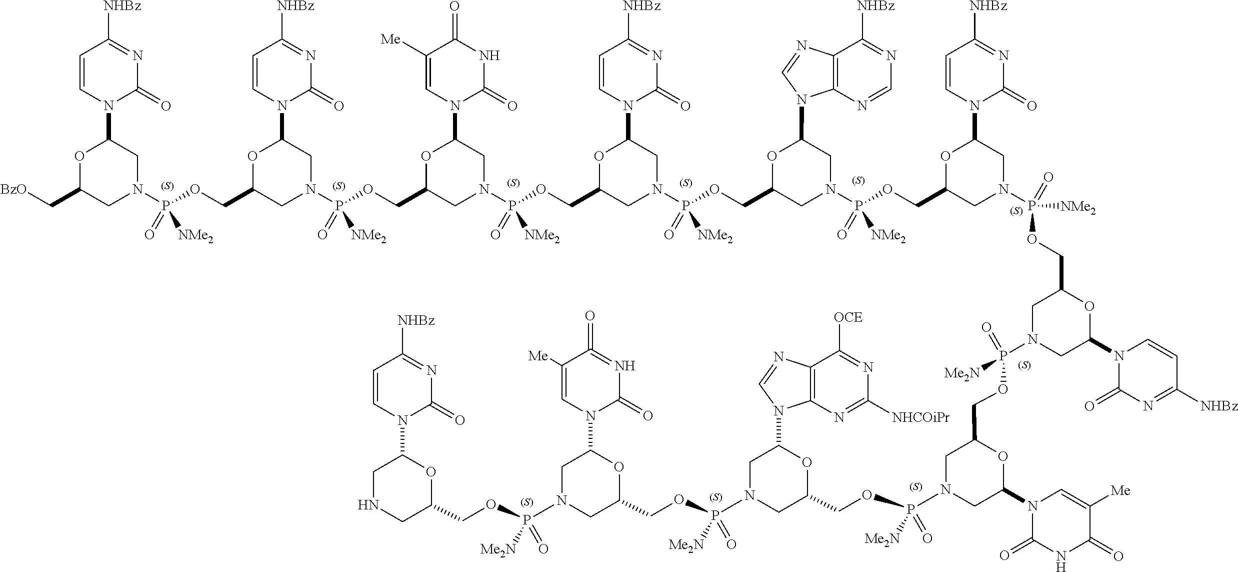

Using General Procedure B: reaction of trityl protected 11-mer (500 mg) afforded free base 11-mer (481 mg).

Coupling to 12-mer:

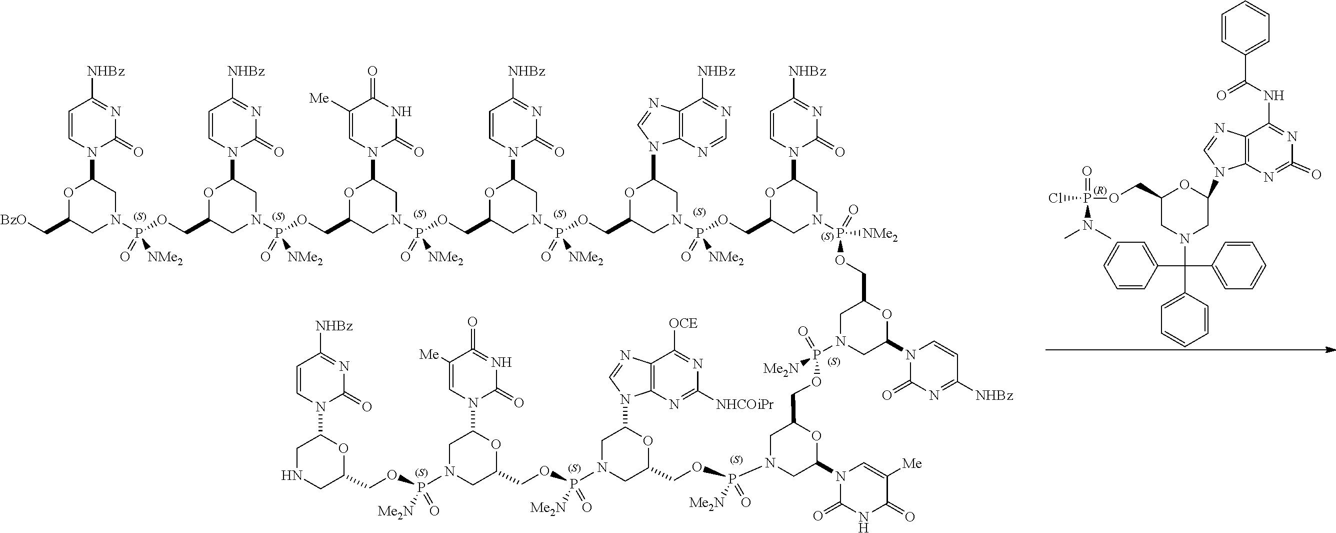

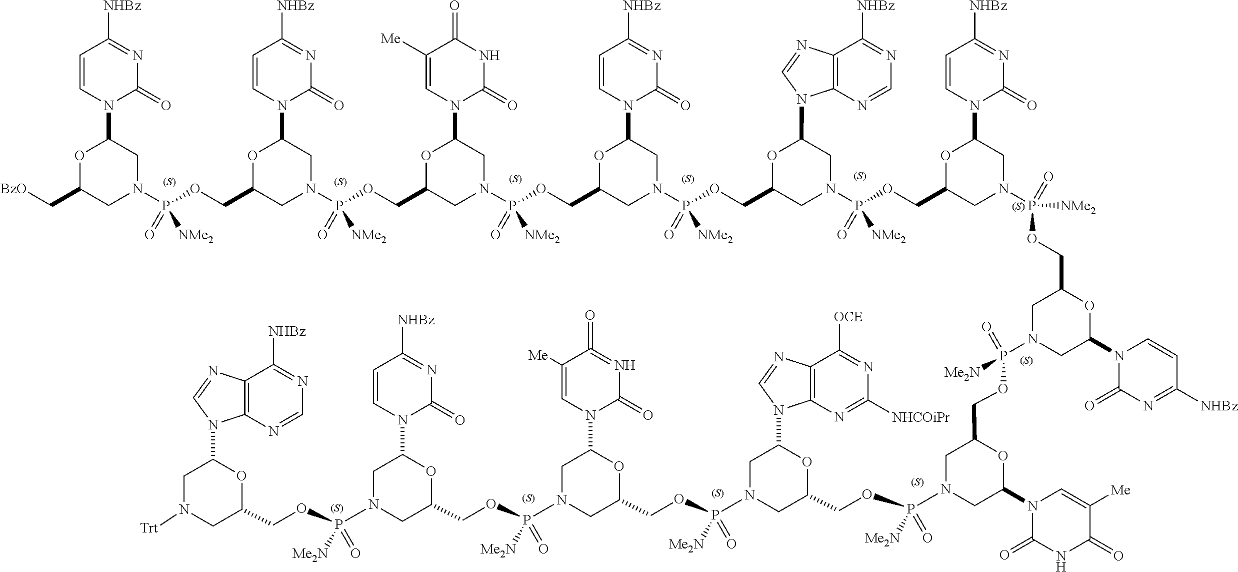

Using General Procedure A: reaction of 11-mer (481 mg) with ((2S,6R)-6-(6-benzamido-9H-purin-9-yl)-4-tritylmorpholin-2-yl)methyl (R)-dimethylphosphoramidochloridate (102 mg) afforded 12-mer (525 mg).

Deprotection of 12-mer:

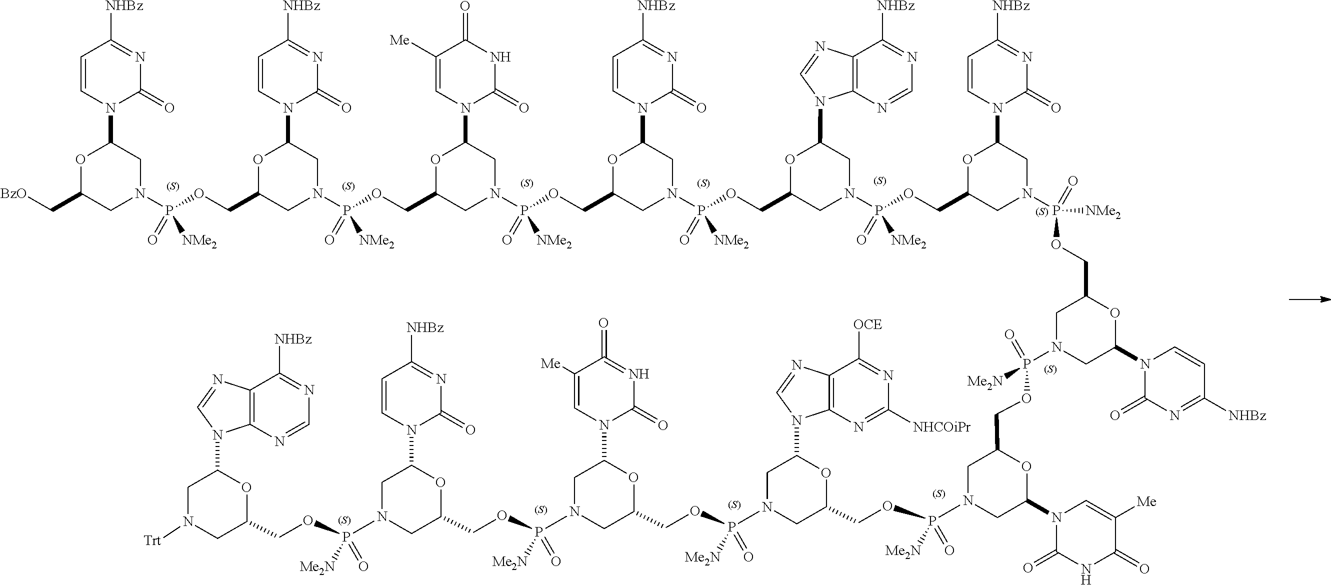

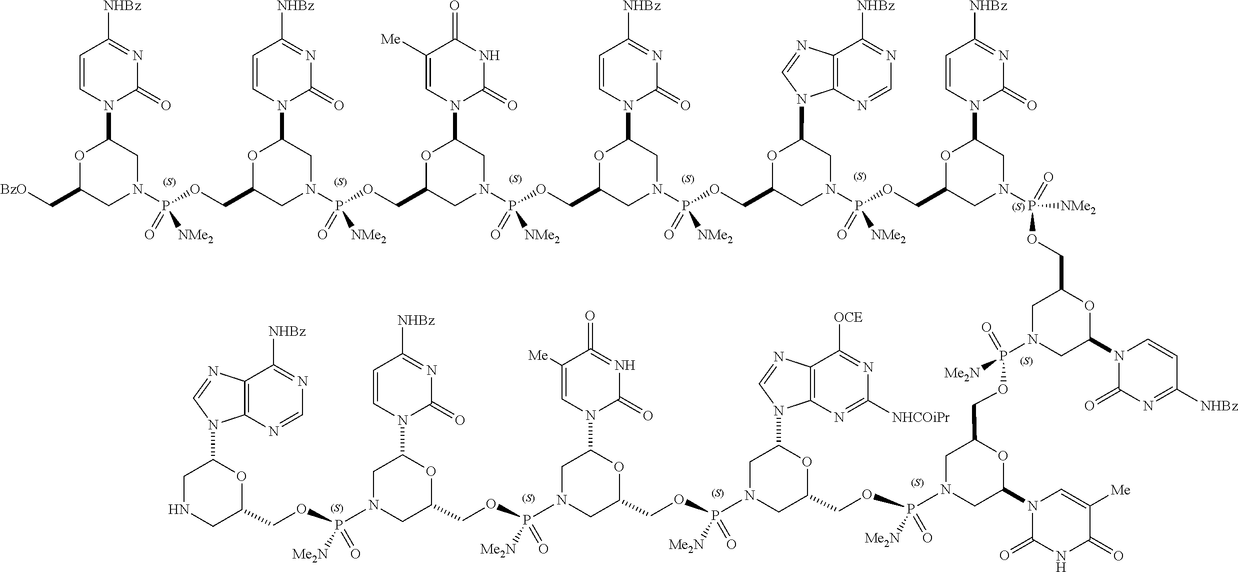

Using General Procedure B: reaction of trityl protected 12-mer (525 mg) afforded free base 12-mer (490 mg).

Coupling to 13-mer:

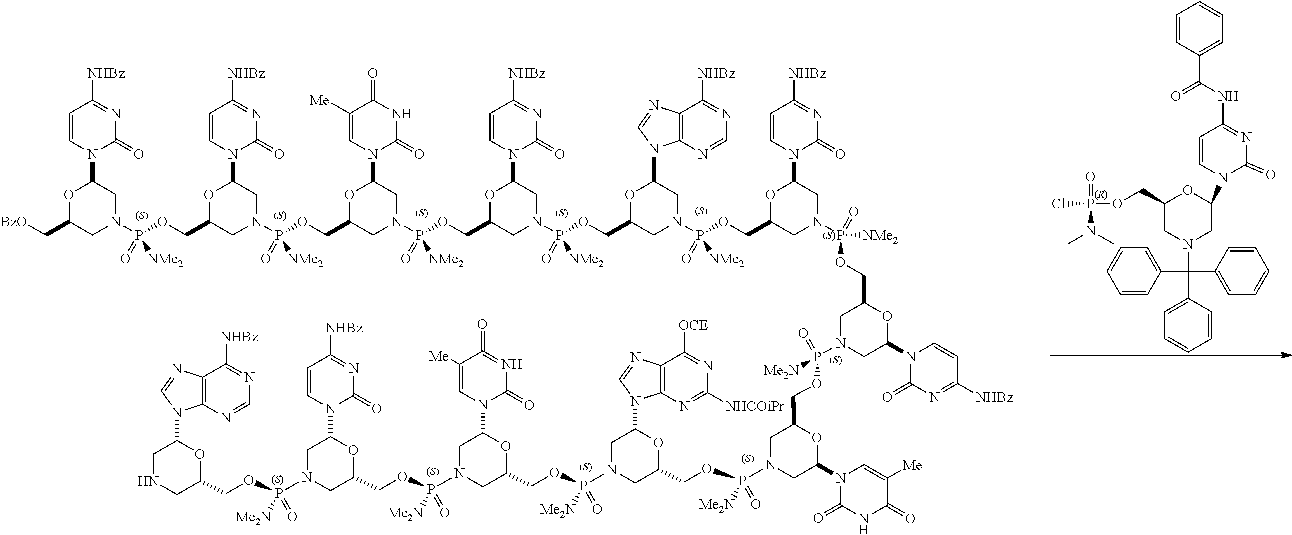

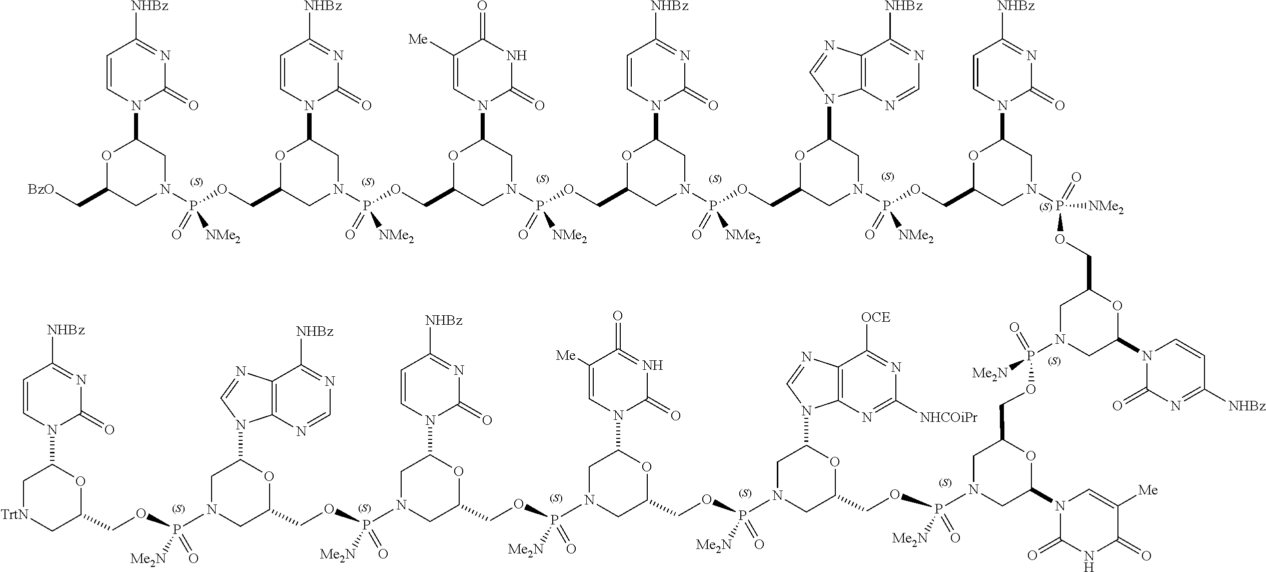

Using General Procedure A: reaction of 12-mer (484 mg) with ((2S,6R)-6-(4-benzamido-2-oxopyrimidin-1(2H)-yl)-4-tritylmorpholin-2-yl)methyl (R)-dimethylphosphoramidochloridate (86 mg) afforded 13-mer (550 mg).

Deprotection of 13-mer:

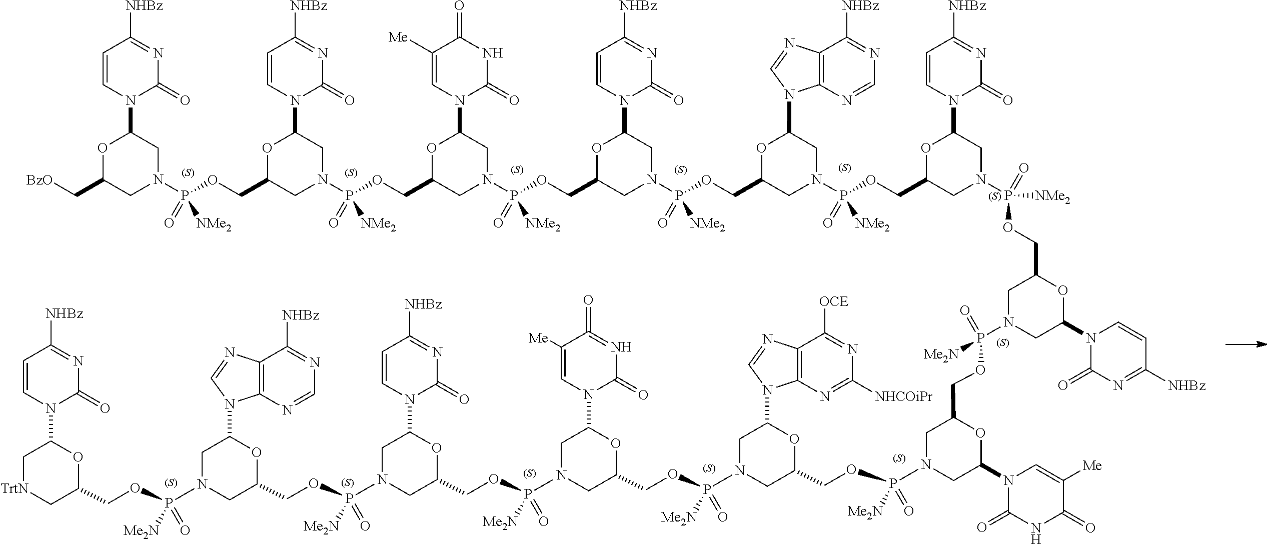

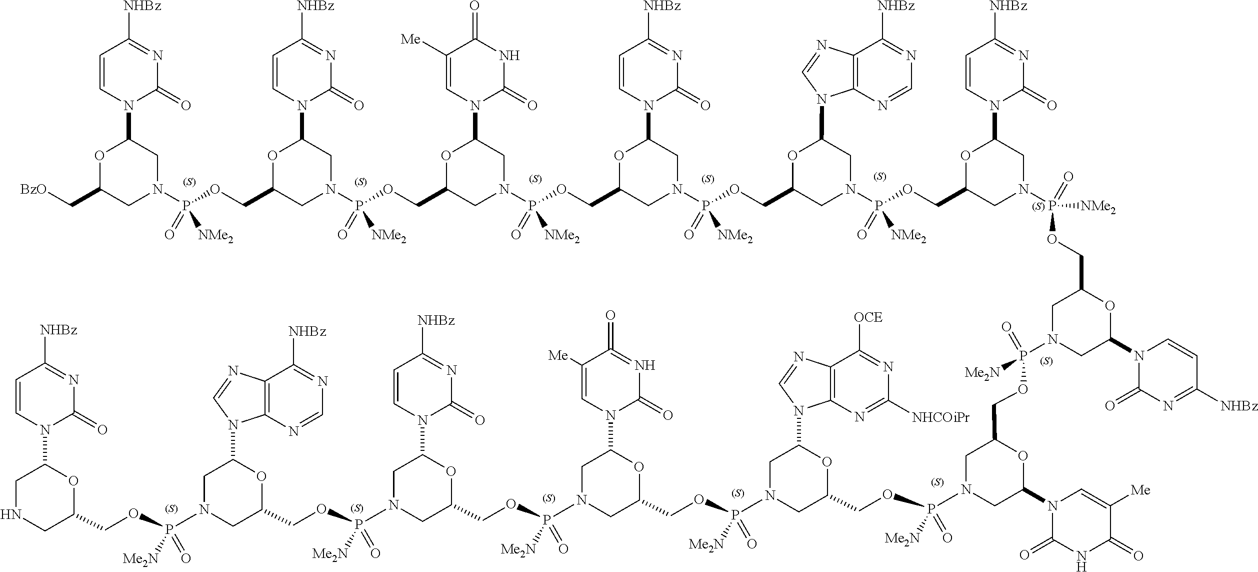

Using General Procedure B: reaction of trityl protected 13-mer (550 mg) afforded free base 13-mer (550 mg).

Coupling to 14-mer:

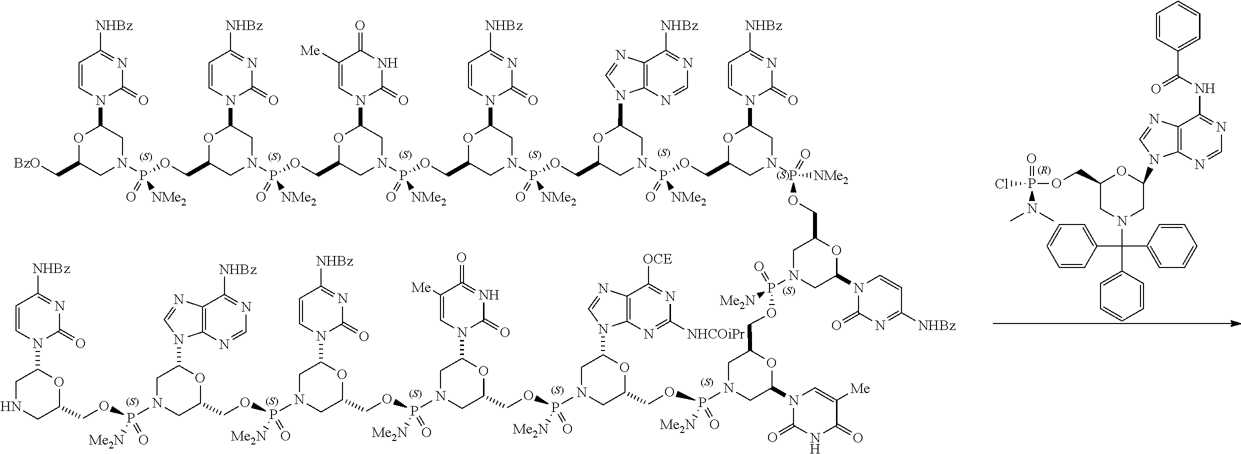

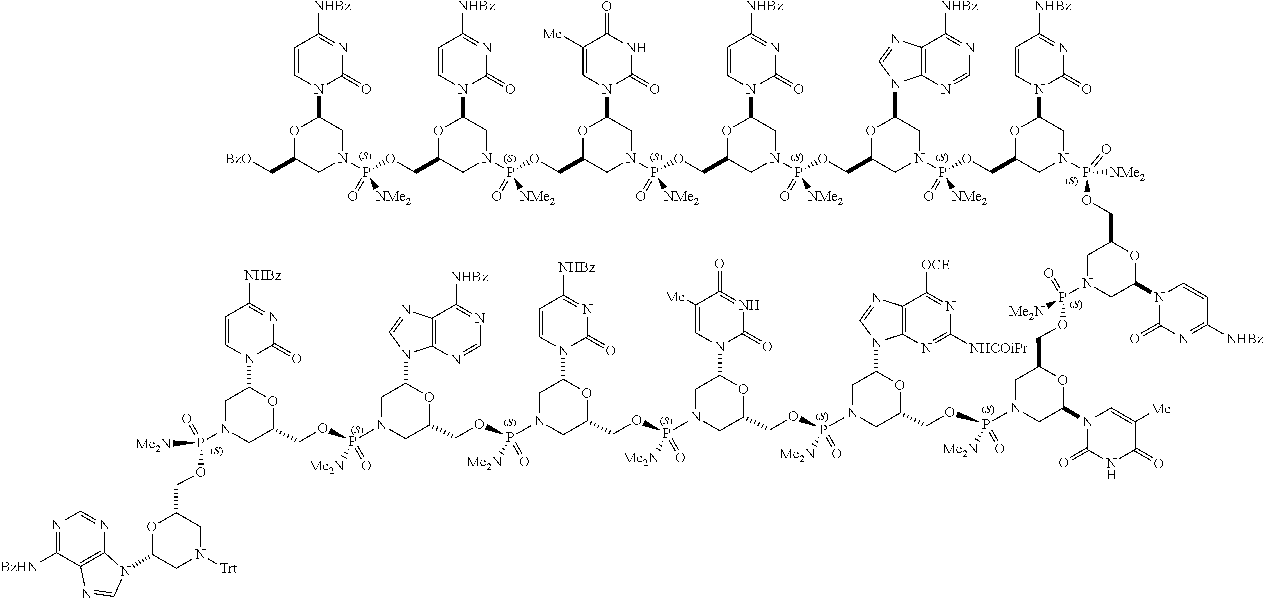

Using General Procedure A: reaction of 13-mer (550 mg) with ((2S,6R)-6-(6-benzamido-9H-purin-9-yl)-4-tritylmorpholin-2-yl)methyl (R)-dimethylphosphoramidochloridate (94 mg) afforded 14-mer (621 mg).

Deprotection of 14-mer:

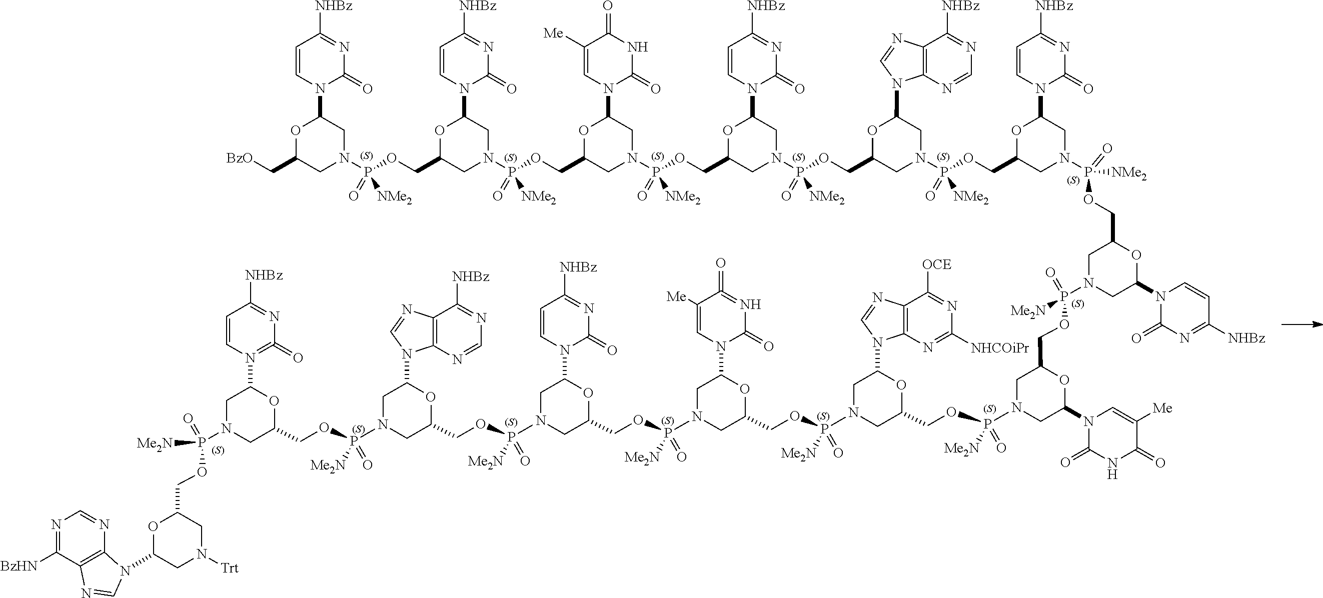

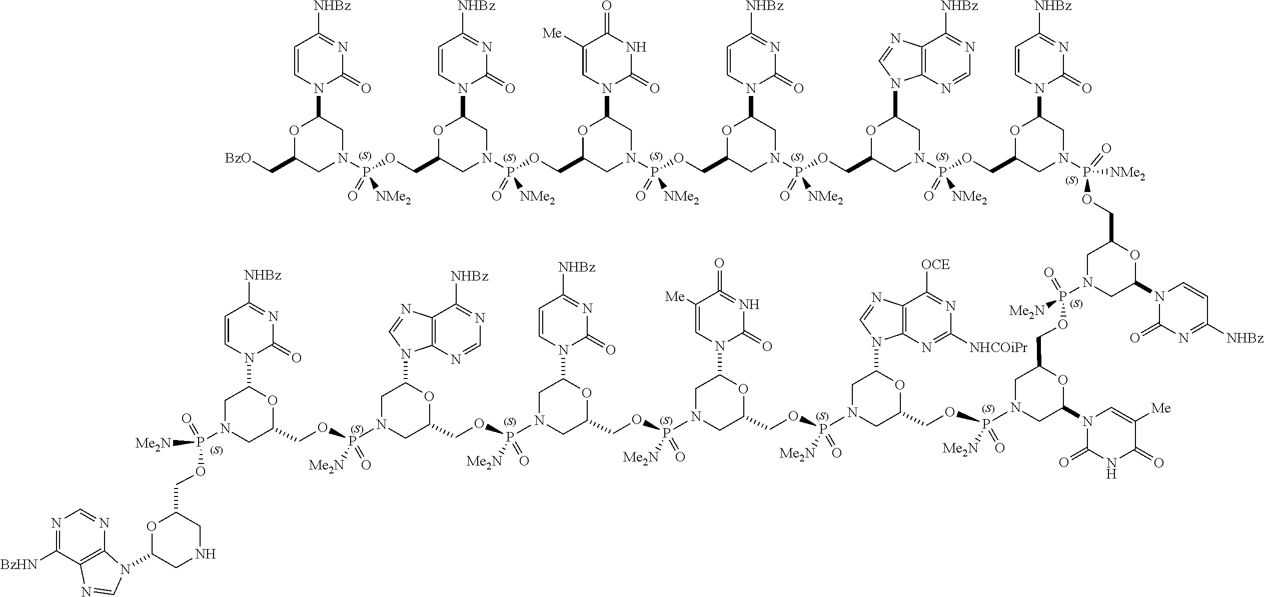

Using General Procedure B: reaction of trityl protected 14-mer (621 mg) afforded free base 14-mer (596 mg).

Coupling to 15-mer:

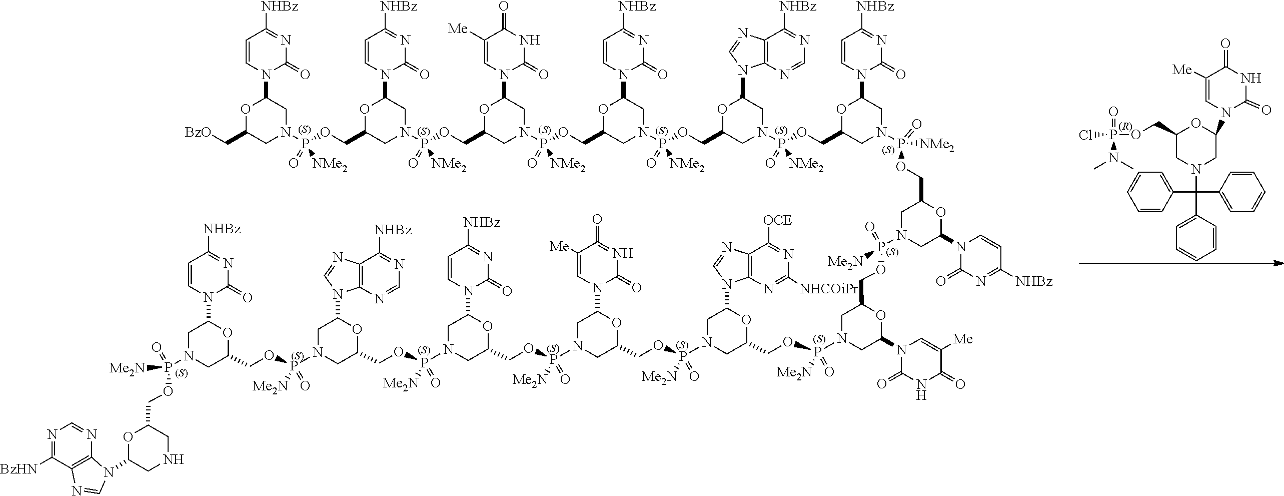

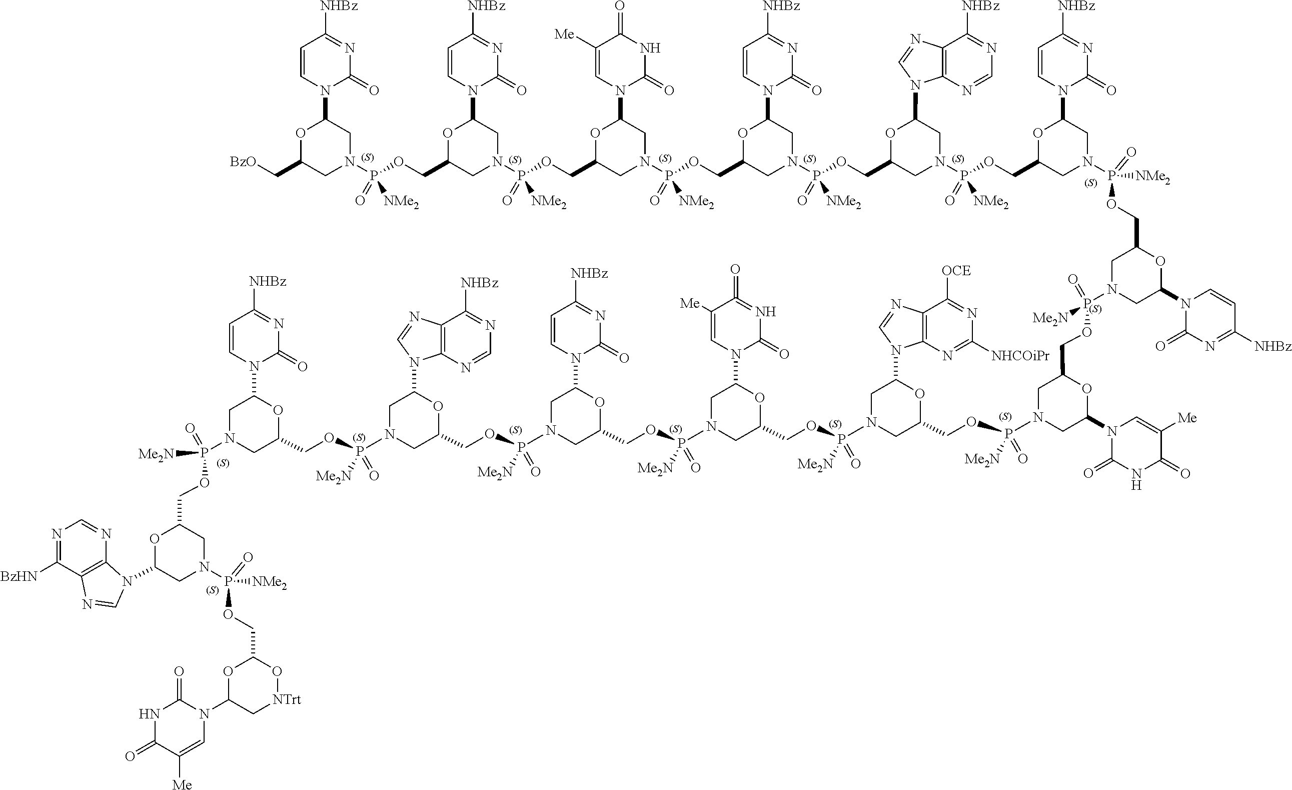

Using General Procedure A: reaction of 14-mer (596 mg) with ((2S,6R)-6-(5-methyl-2,4-dioxo-3,4-dihydropyrimidin-1(2H)-yl)-4-tritylmorpholin-2-yl)methyl (R)-dimethylphosphoramidochloridate (84 mg) afforded 15-mer (655 mg). MS (ESI) m/z: Calcd for C₂₇₄H₃₃₇N₇₉O₇₂P₁₄: [M+4H/4]⁺ 1581.28. Found: 1582.

Deprotection of 15-mer:

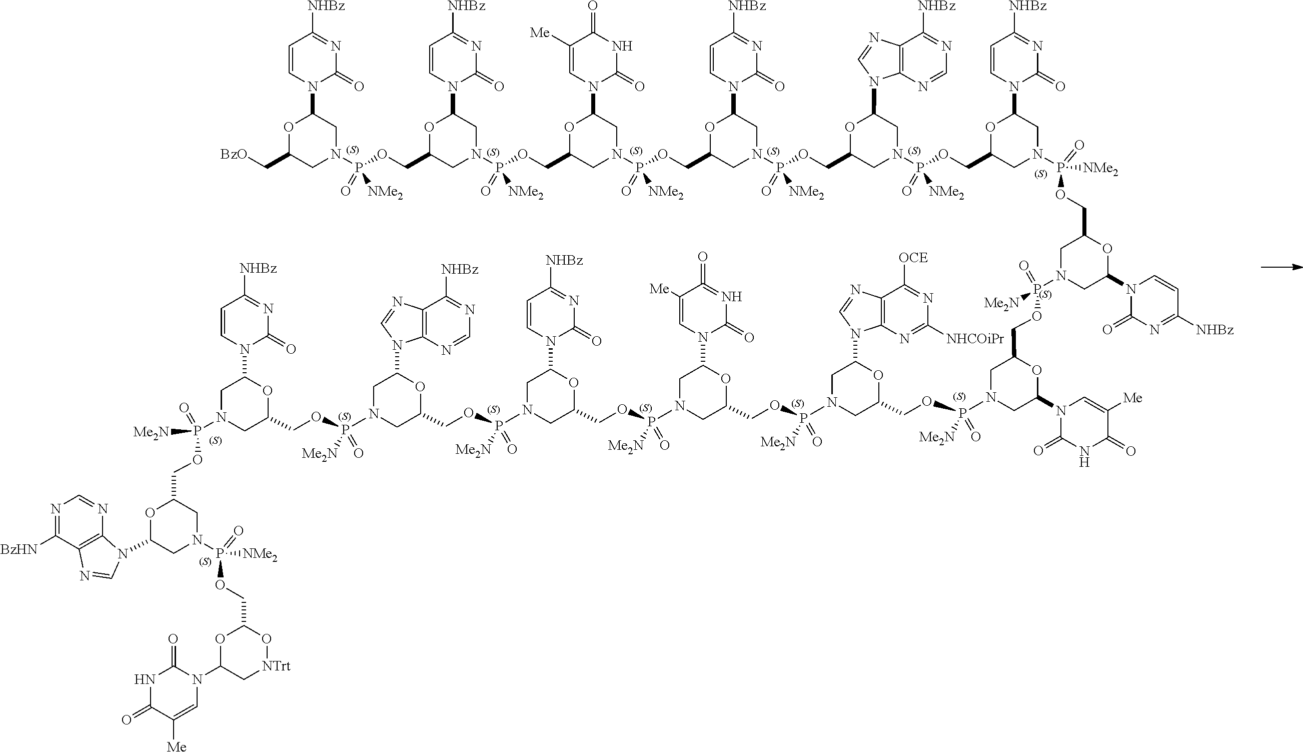

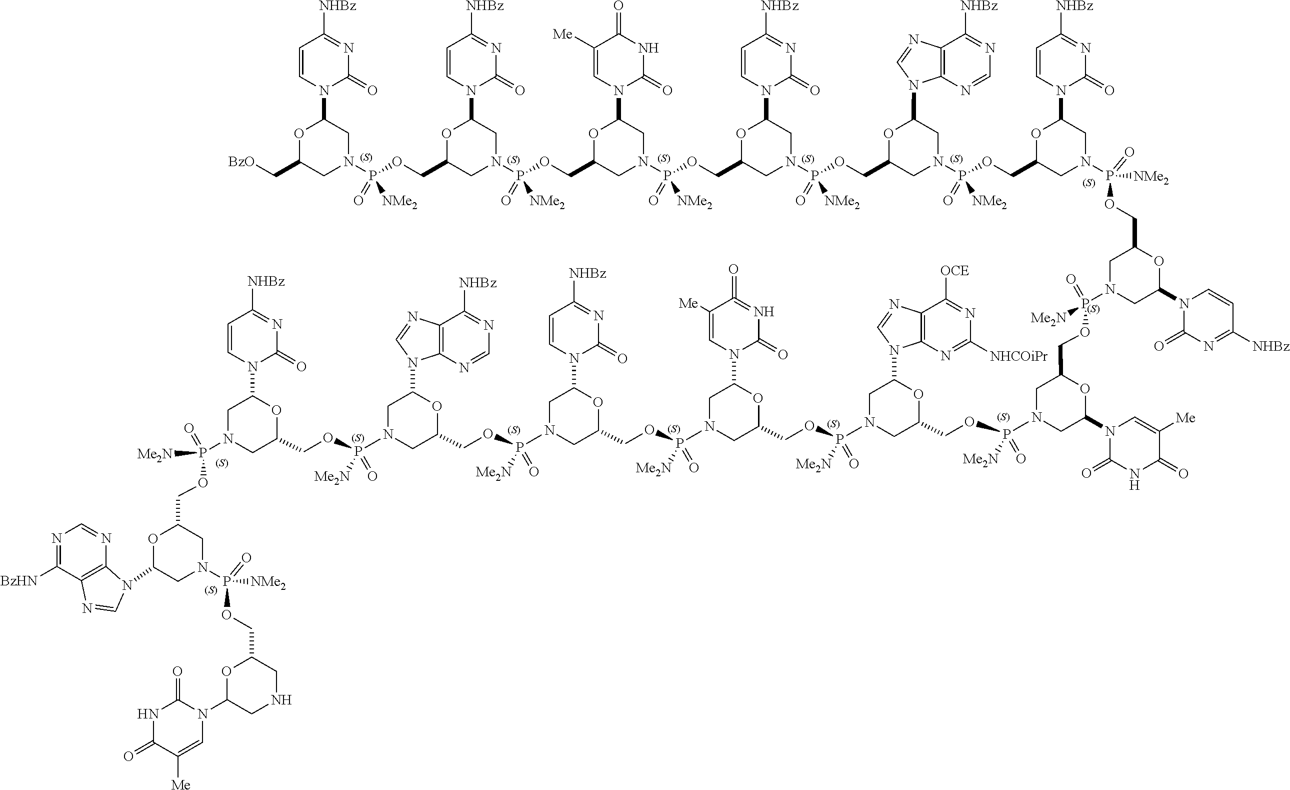

Using General Procedure B: reaction of trityl protected 15-mer (650 mg) afforded free base 15-mer (613 mg). MS (ESI) m/z: Calcd for C₂₅₅H₃₂₃N₇₉O₇₂P₁₄: [M+4H/4]⁺ 1520.76. Found: 1521.

Coupling to 16-mer:

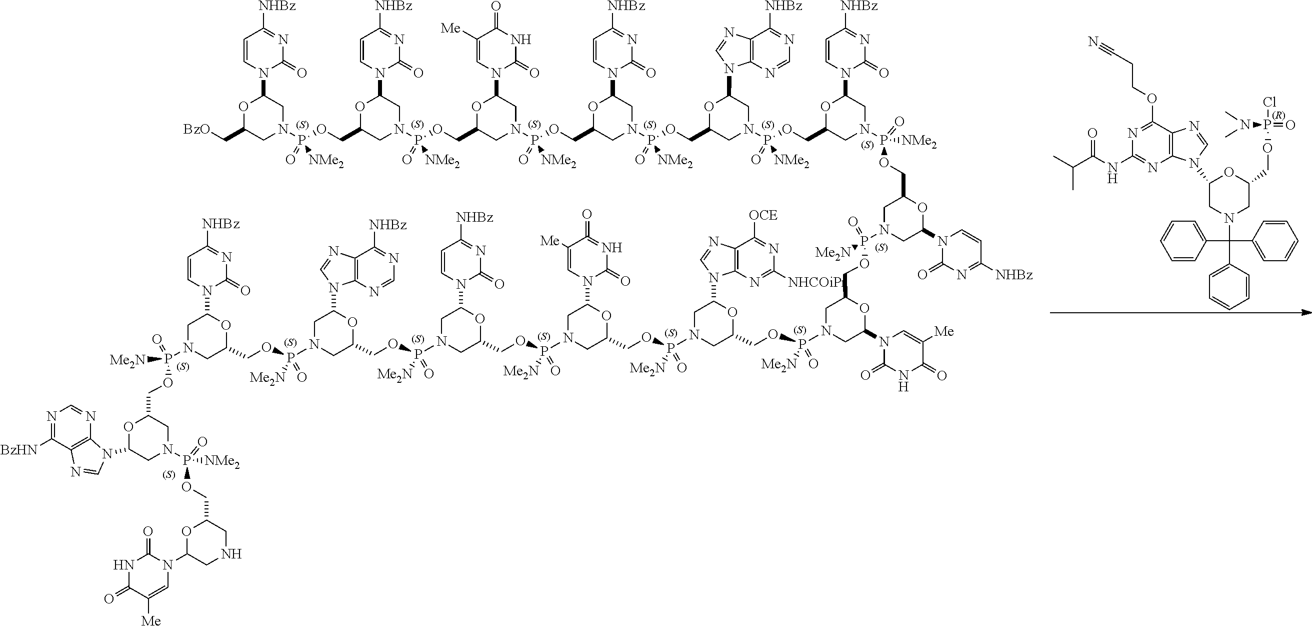

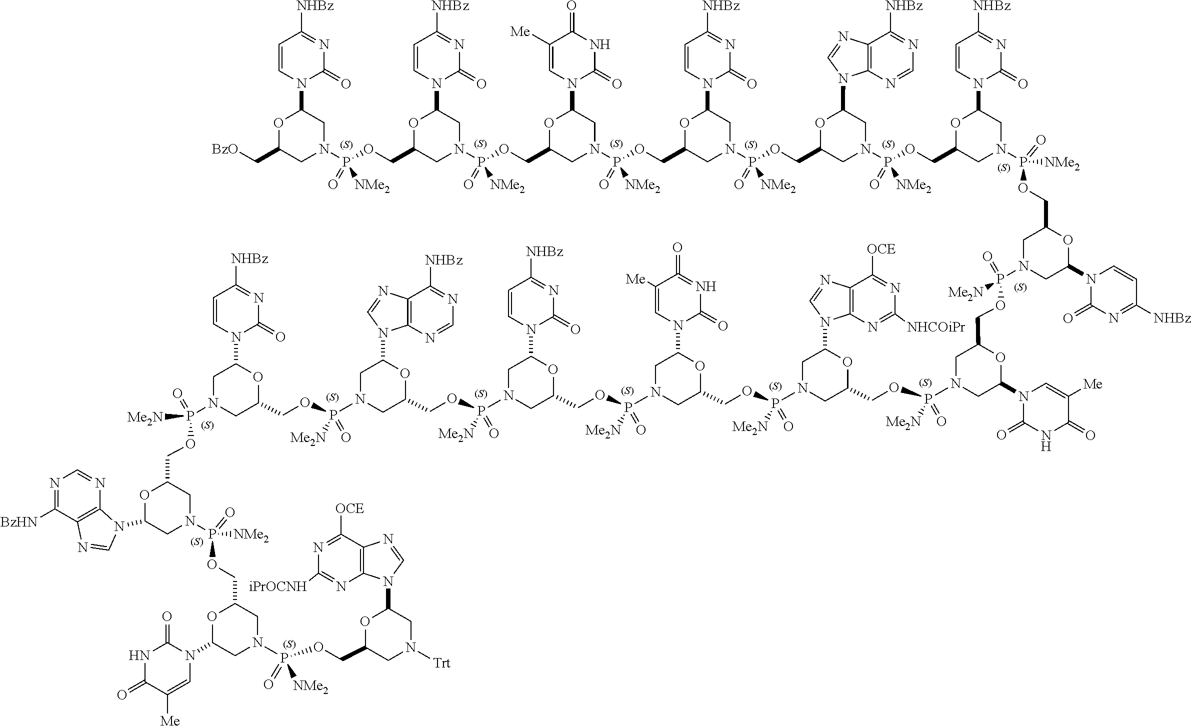

Using General Procedure B: reaction of trityl protected 16-mer (650 mg) afforded free base 16-mer (613 mg). MS (g) afforded for (680 mg

Coupling to 16-mer:

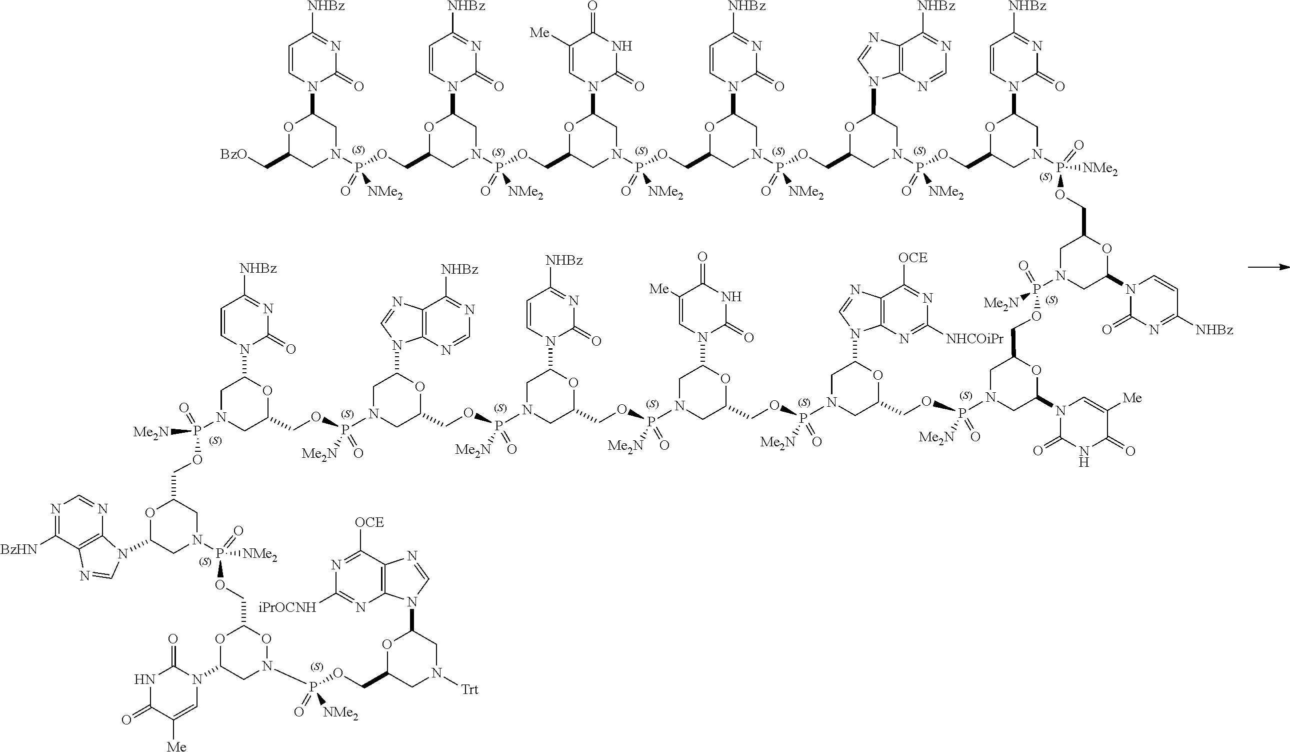

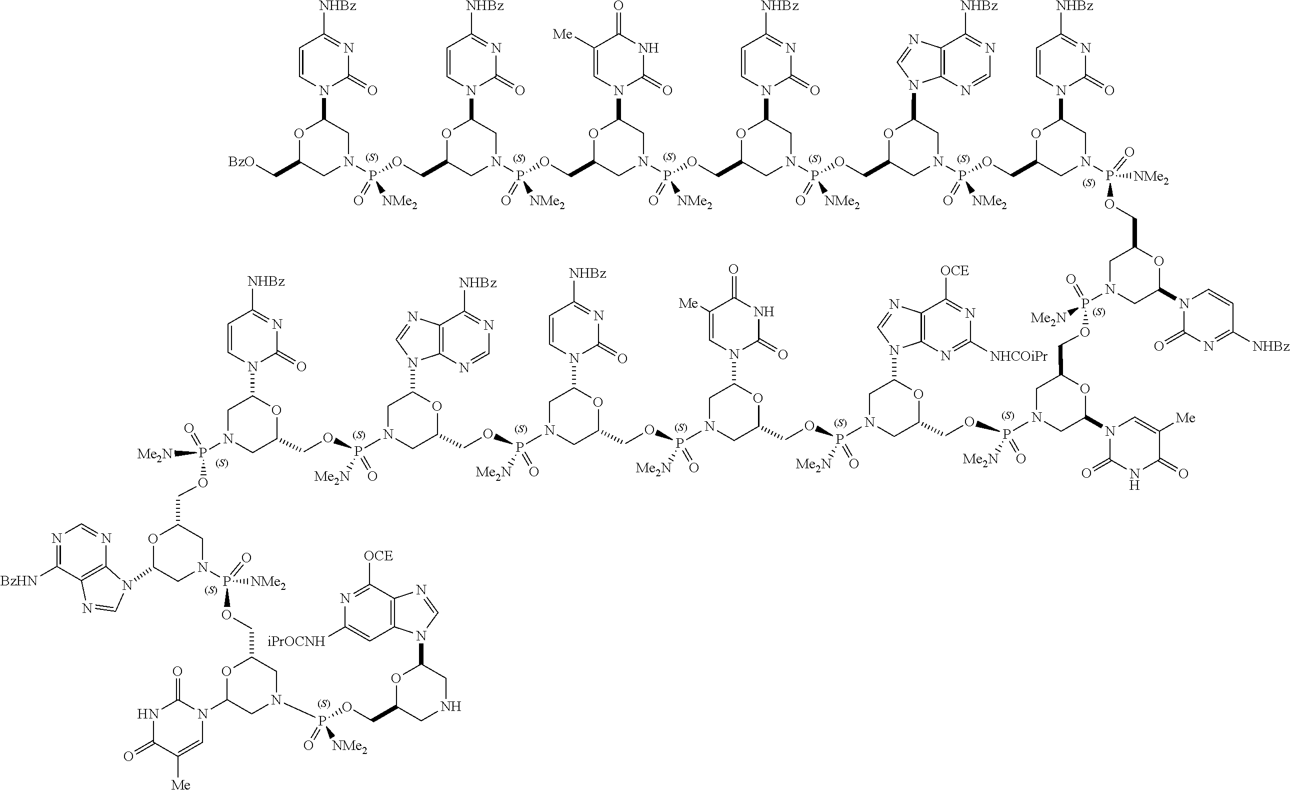

Using General Procedure B: reaction of trityl protected 16-mer (680 mg) afforded free base 16-mer (623 mg).

Coupling to 17-mer:

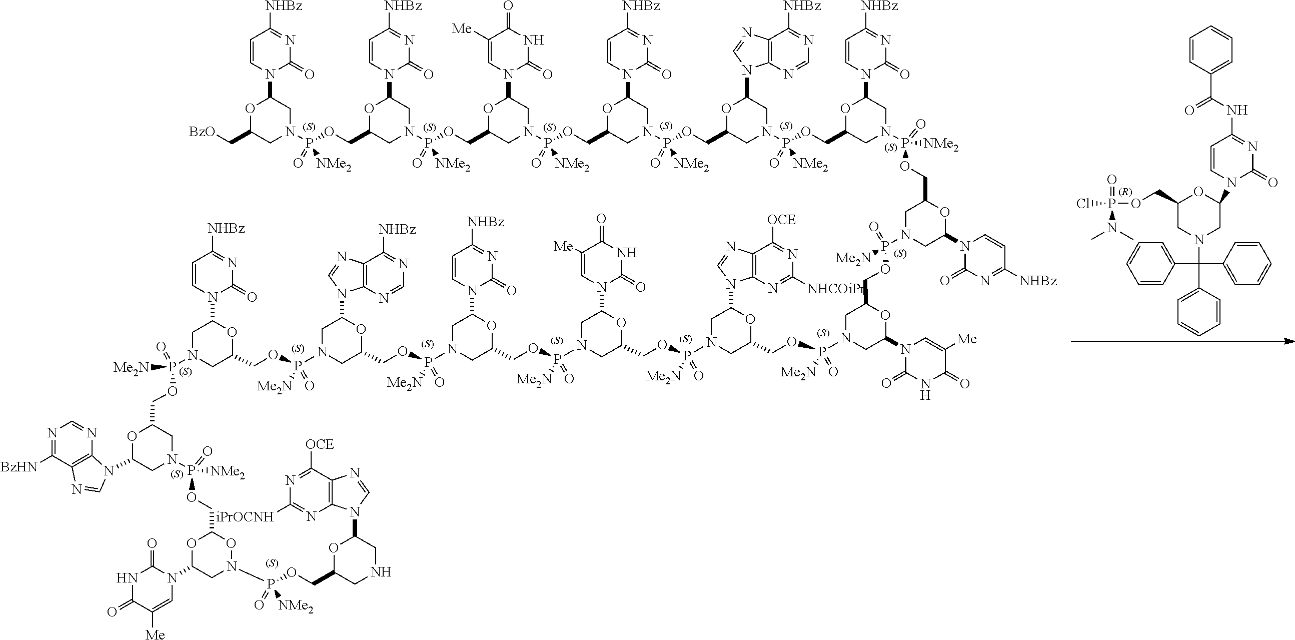

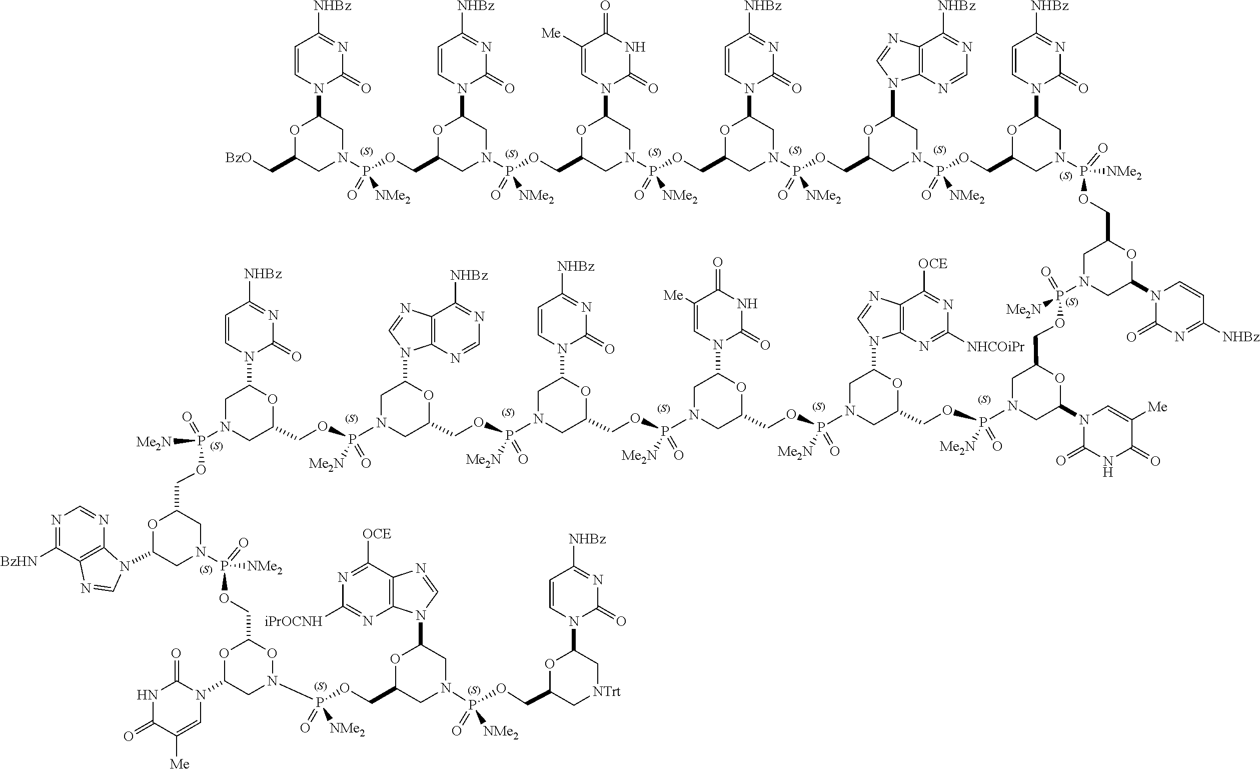

Using General Procedure A: reaction of 16-mer (623 mg) with ((2S,6R)-6-(4-benzamido-2-oxopyrimidin-1(2H)-yl)-4-tritylmorpholin-2-yl)methyl (R)-dimethylphosphoramidochloridate (93 mg) afforded 17-mer (690 mg).

Deprotection of 17-mer:

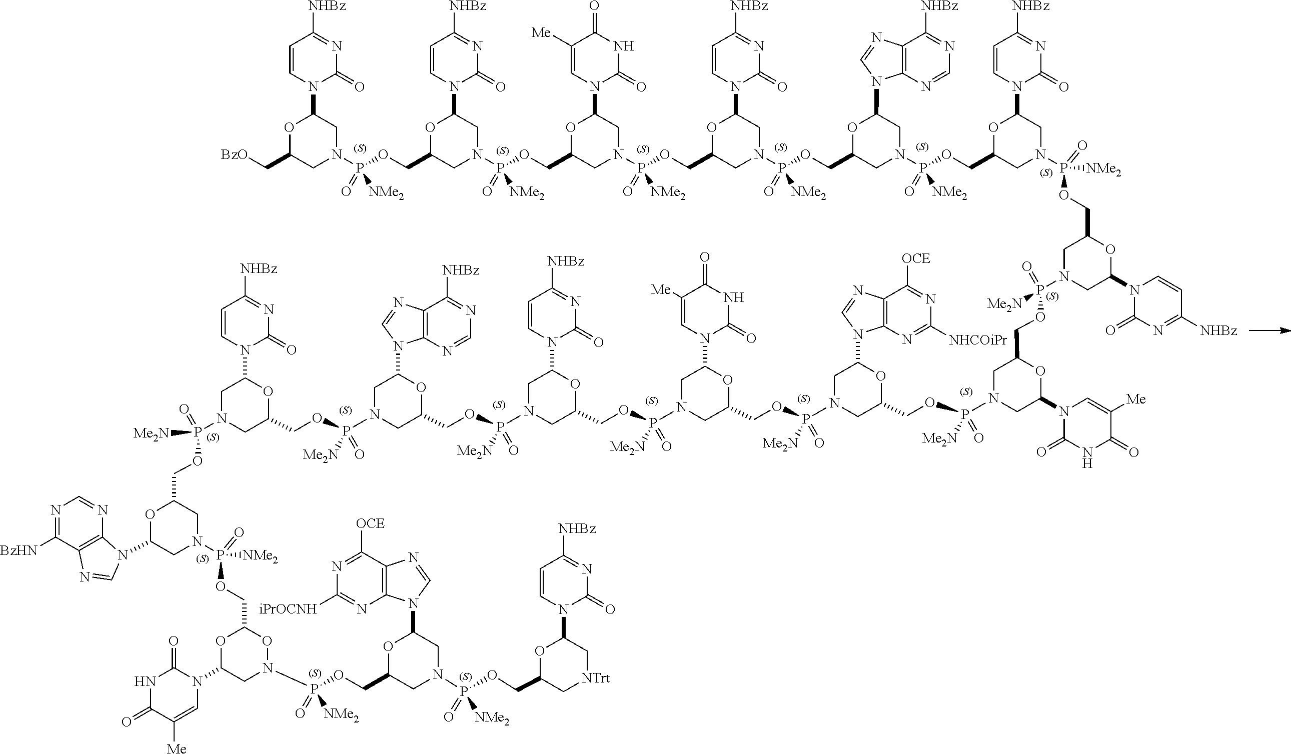

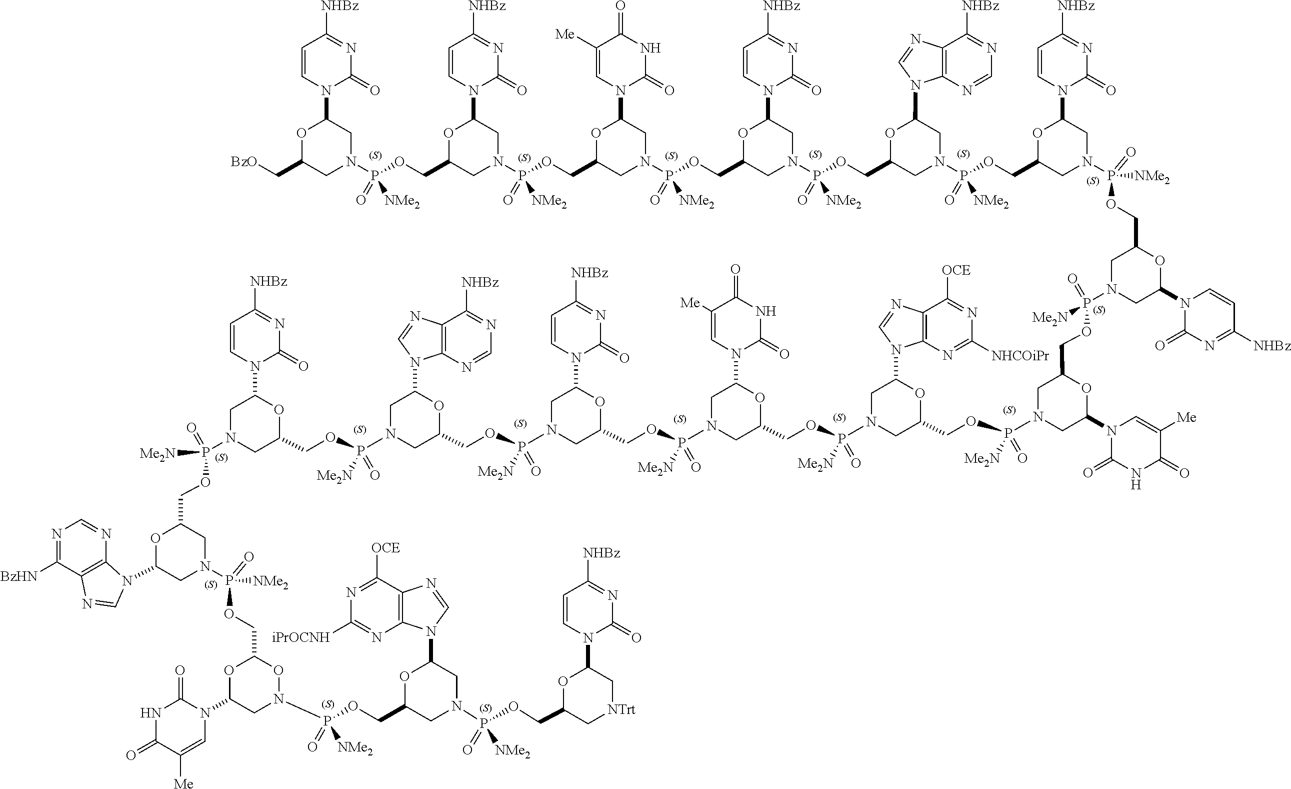

Using General Procedure B: reaction of trityl protected 17-mer (690 mg) afforded free base 17-mer (670 mg).

Coupling to 18-mer:

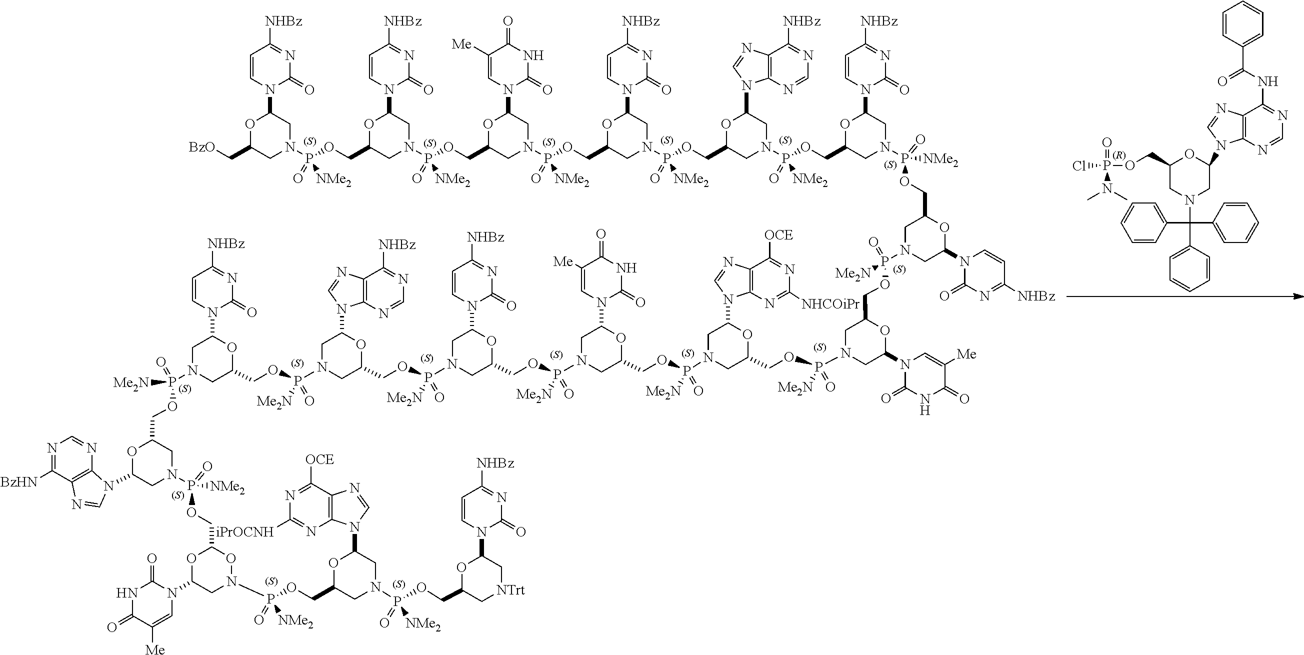

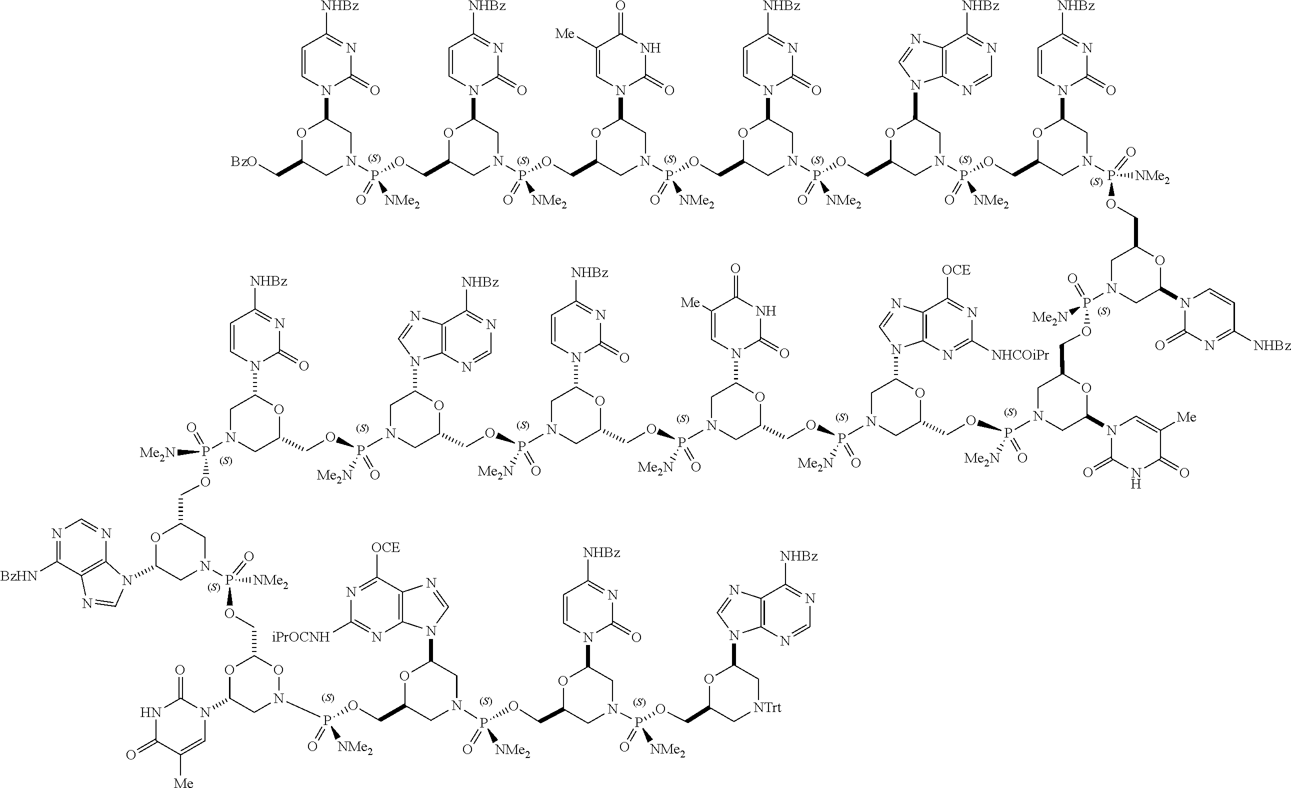

Using General Procedure A: reaction of 17-mer (673 mg) with ((2S,6R)-6-(6-benzamido-9H-purin-9-yl)-4-tritylmorpholin-2-yl)methyl (R)-dimethylphosphoramidochloridate (98 mg) afforded 18-mer (740 mg).

Deprotection of 18-mer:

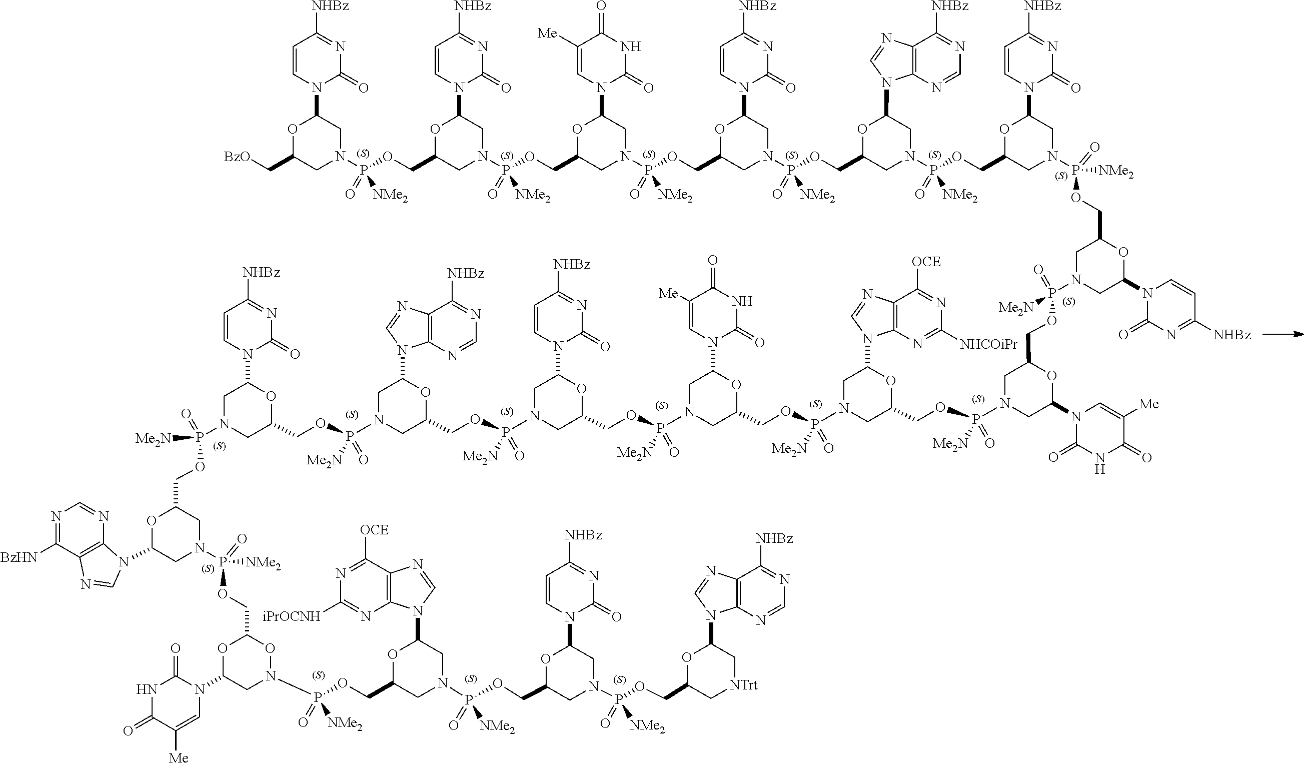

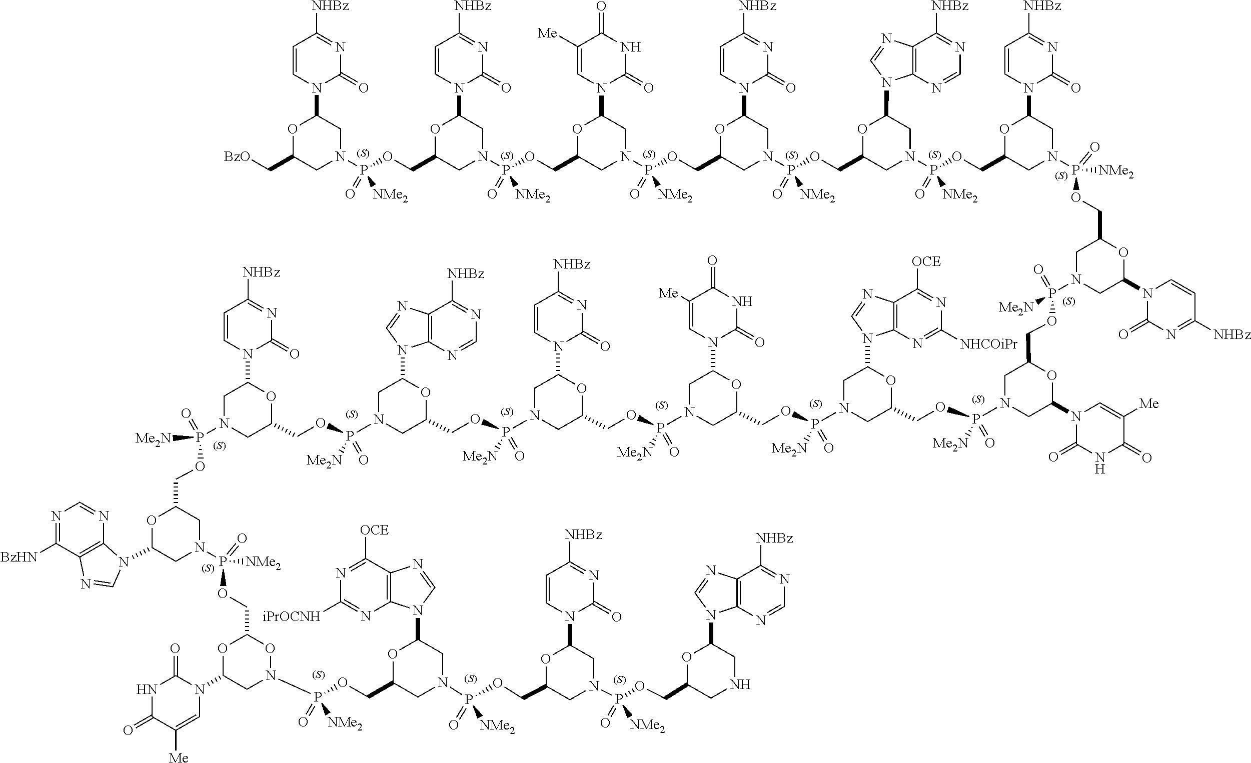

Using General Procedure B: reaction of trityl protected 18-mer (739 mg) afforded free base 18-mer (675 mg).

Coupling to 19-mer:

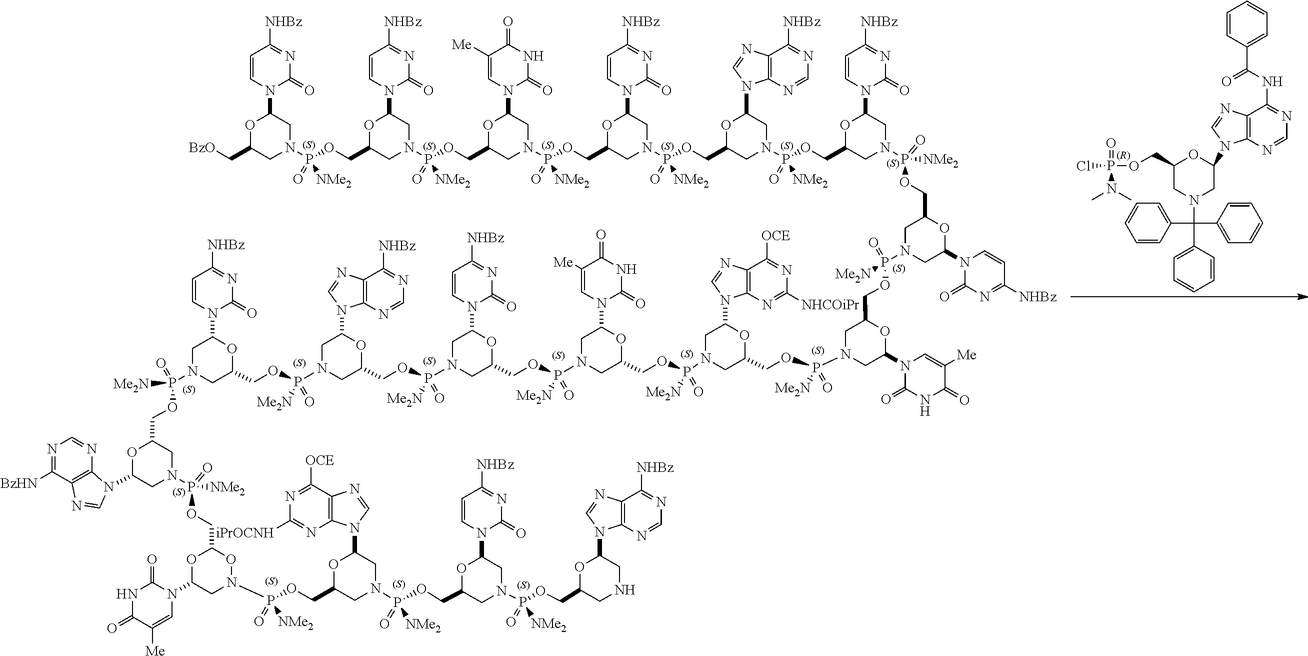

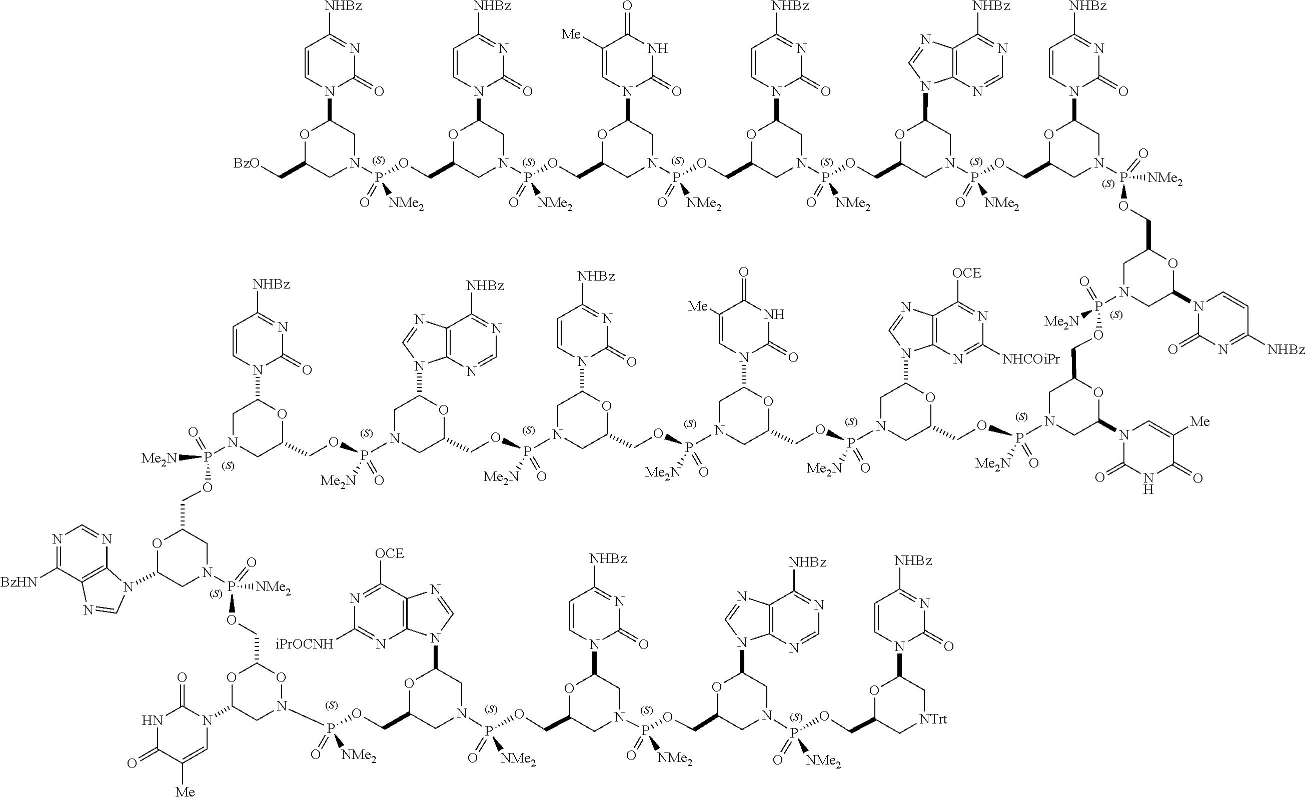

Using General Procedure A: reaction of 18-mer (675 mg) with ((2S,6R)-6-(4-benzamido-2-oxopyrimidin-1(2H)-yl)-4-tritylmorpholin-2-yl)methyl (R)-dimethylphosphoramidochloridate (127 mg) afforded 19-mer (735 mg).

Deprotection of 19-mer:

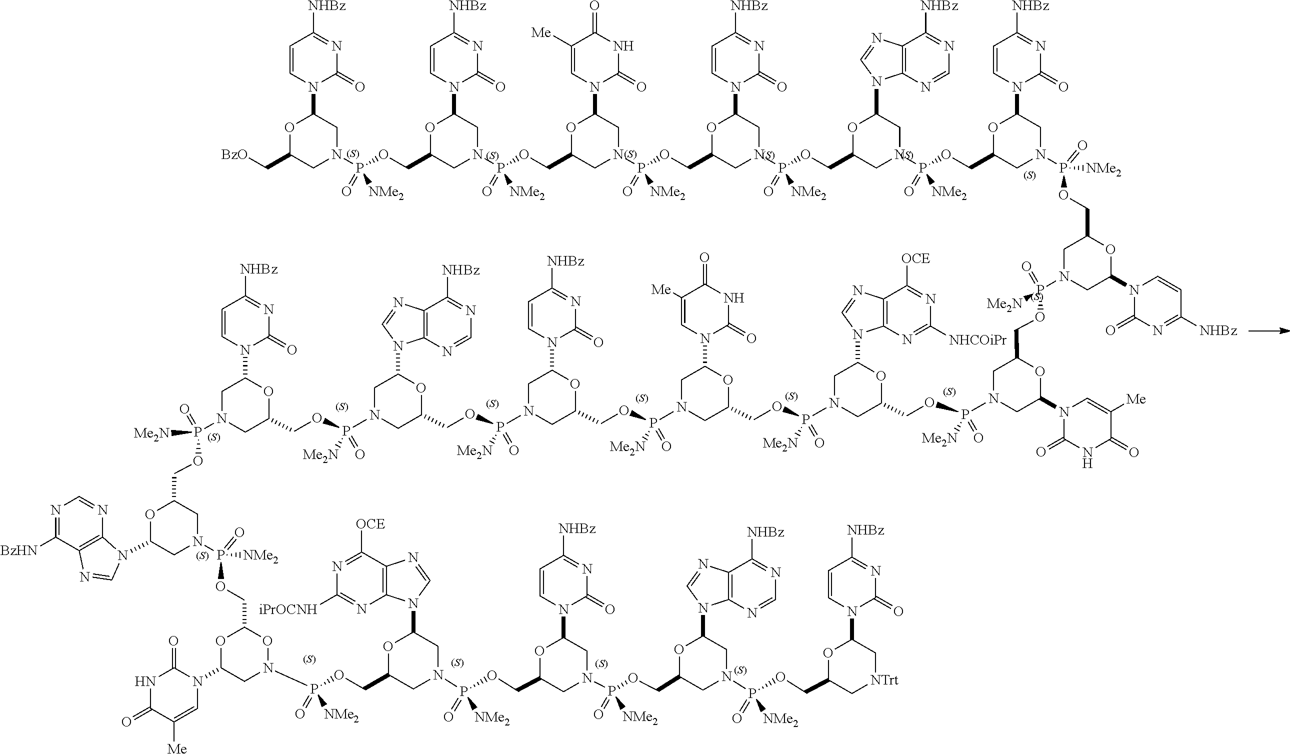

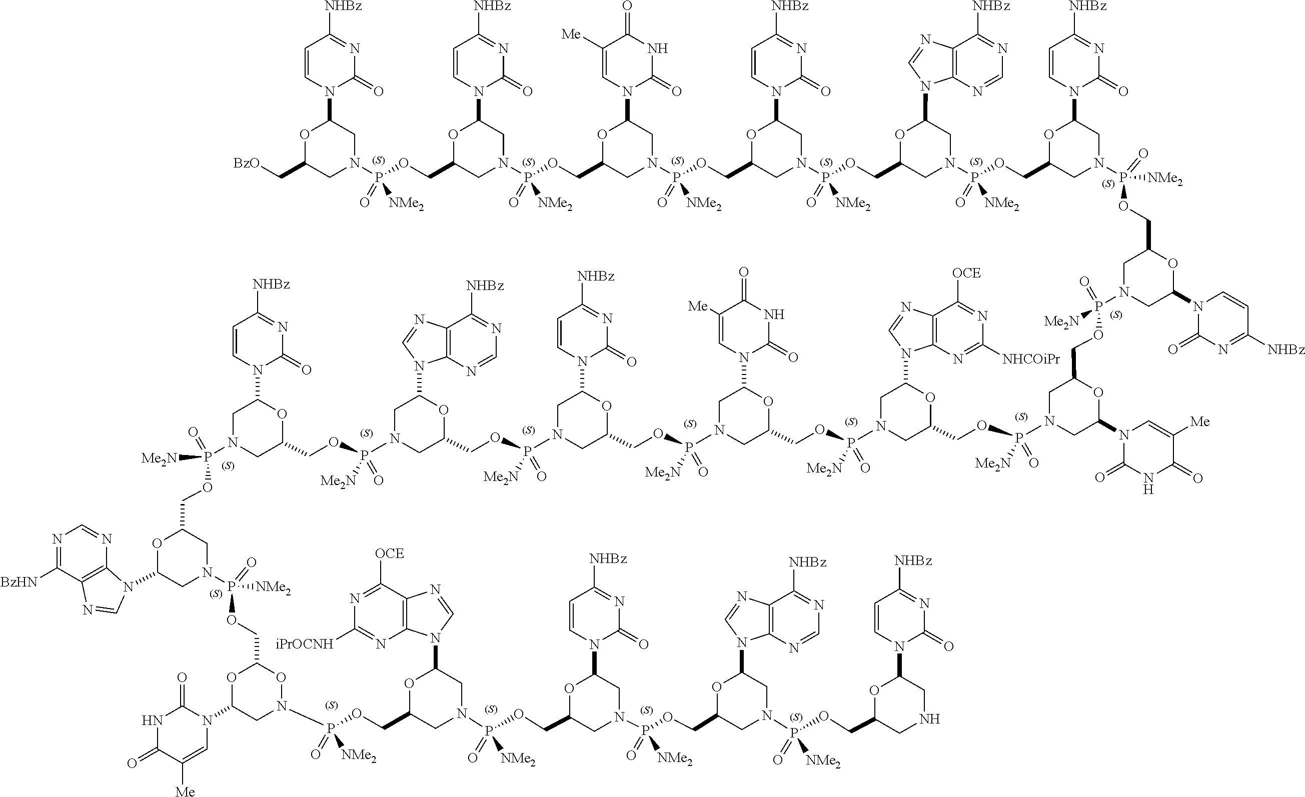

Using General Procedure B: reaction of trityl protected 19-mer (735 mg) afforded free base 19-mer (732 mg).

Coupling to 20-mer:

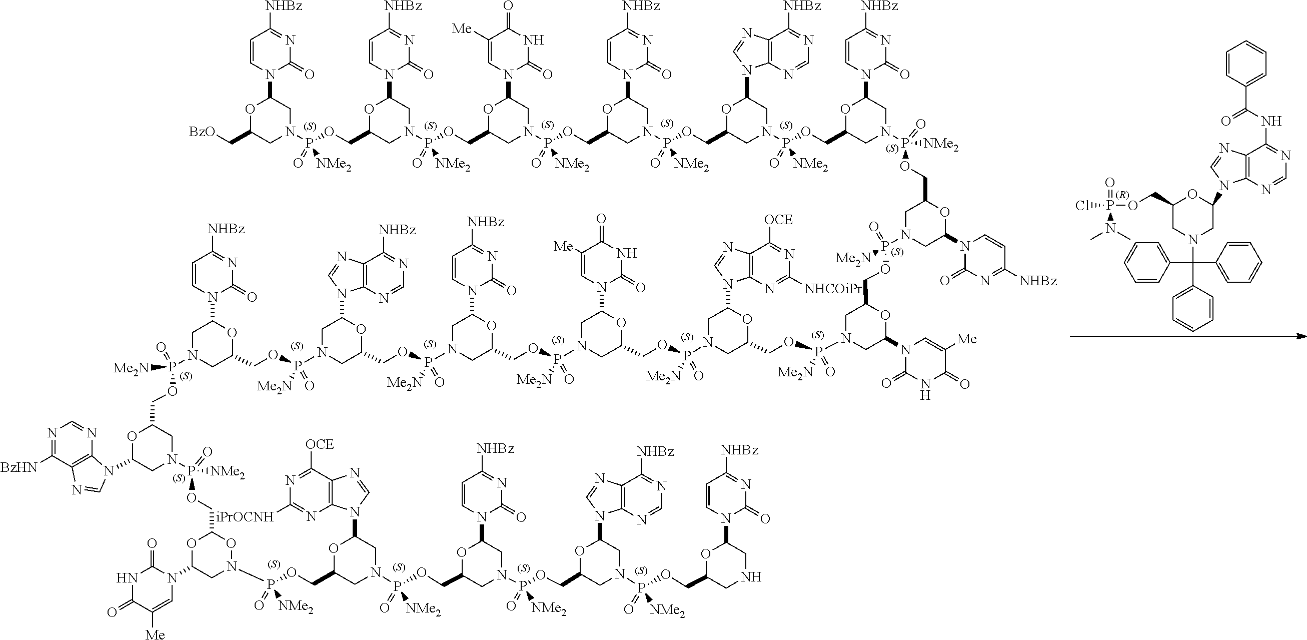

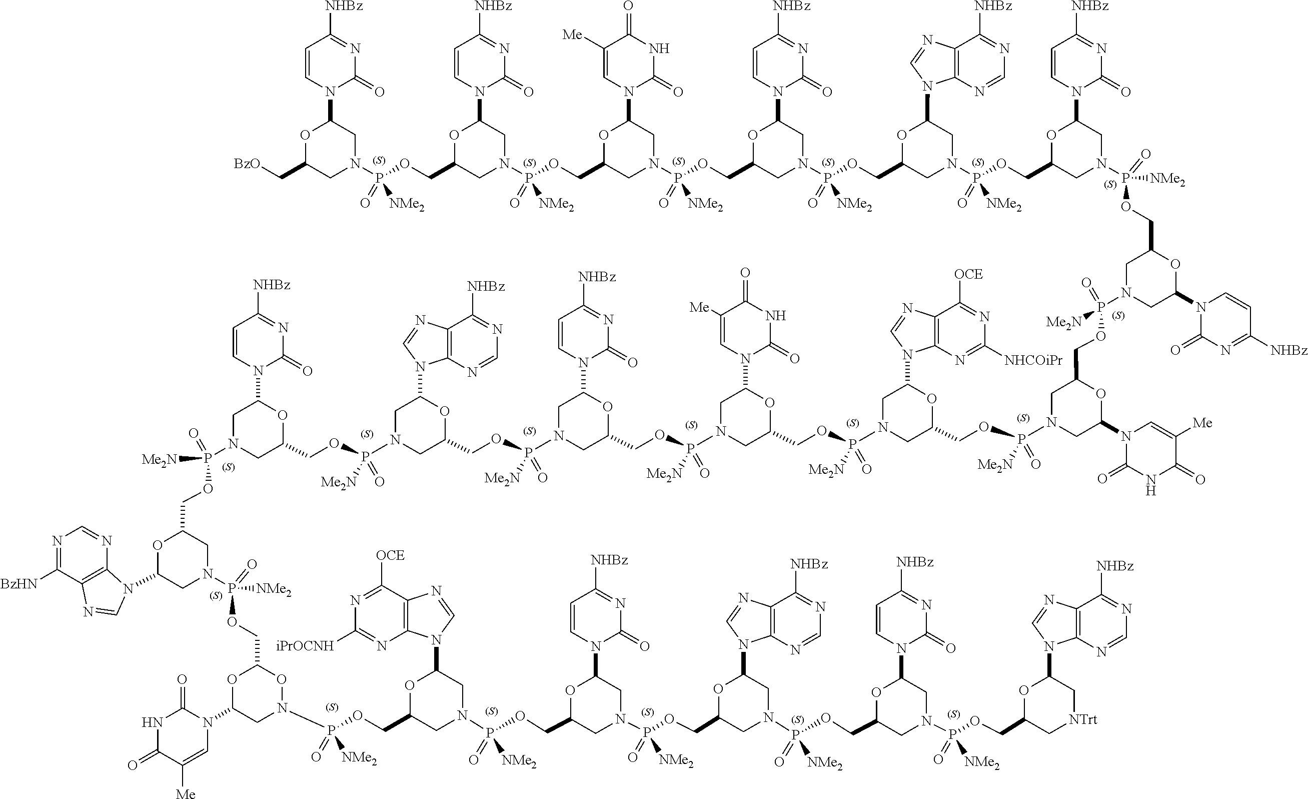

Using General Procedure A: reaction of 19-mer (732 mg) with ((2S,6R)-6-(6-benzamido-9H-purin-9-yl)-4-tritylmorpholin-2-yl)methyl (R)-dimethylphosphoramidochloridate (135 mg) afforded 20-mer (790 mg). MS (ESI) m/z: Calcd for C₃₆₇H₄₅₂N₁₁₁O₉₅P₁₉: [M+5H/5]⁺ 1706. Found: 1707.

Deprotection of 20-mer:

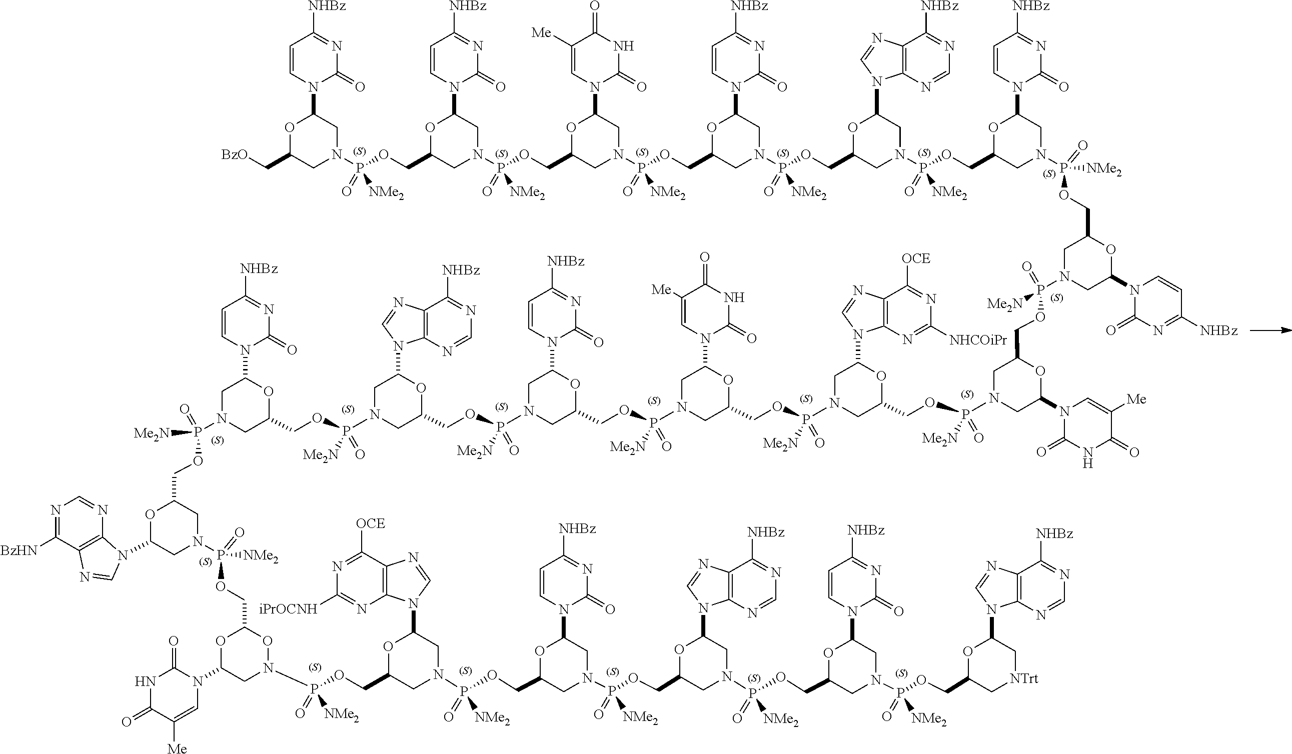

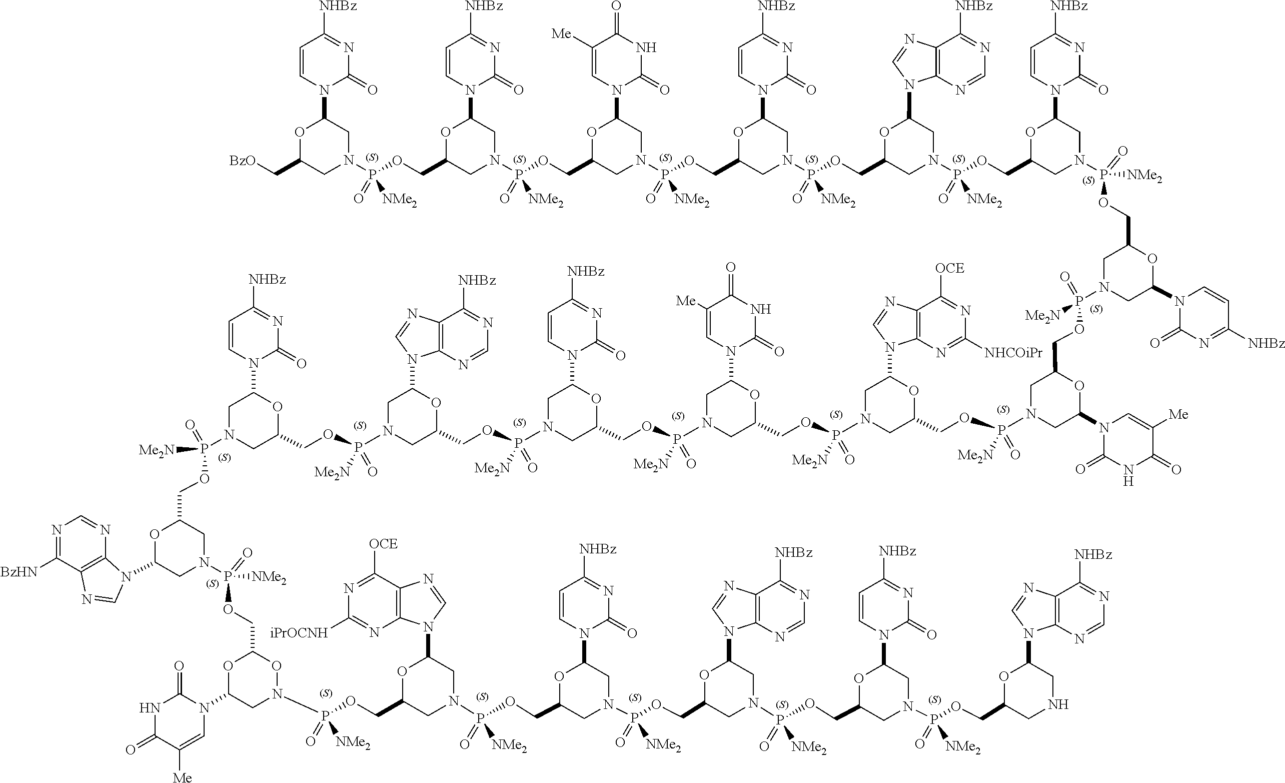

Using General Procedure B: reaction of trityl protected 20-mer (790 mg) afforded free base 20-mer (743 mg). MS (ESI) m/z: Calcd for C₃₄₈H₄₃₈N₁₁₁O₉₅P₁₉: [M+5H/5]⁺ 1657.6. Found: 1658.

Coupling to 21-mer:

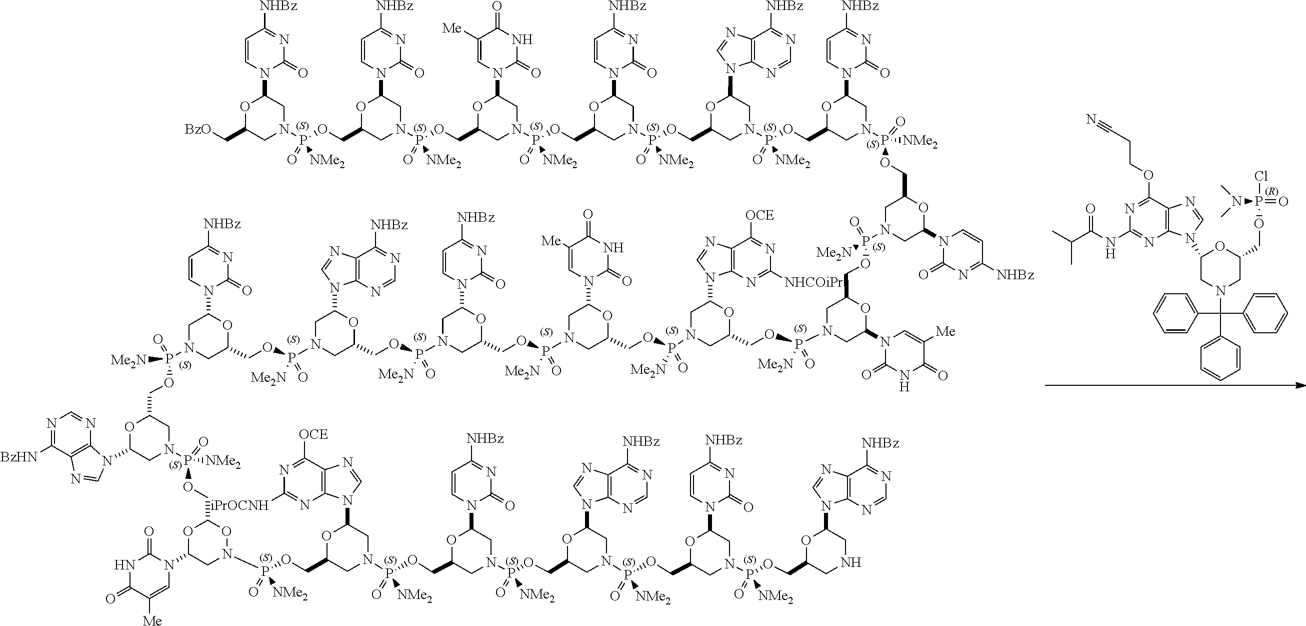

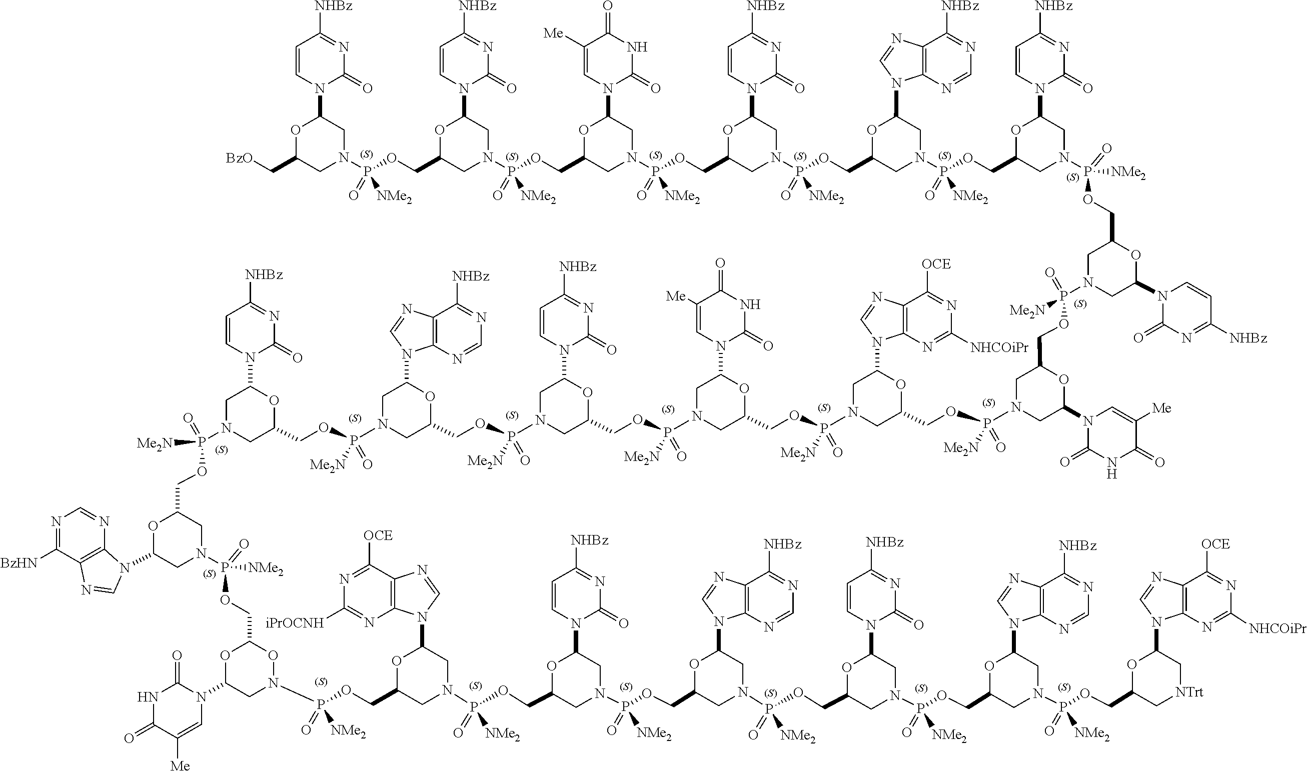

Using General Procedure A: reaction of 20-mer (743 mg) with ((2S,6R)-6-(6-(2-cyanoethoxy)-2-isobutyramido-9H-purin-9-yl)-4-tritylmorpholin-2-yl)methyl (R)-dimethylphosphoramidochloridate (129 mg) afforded 21-mer (795 mg).

Deprotection of 21-mer:

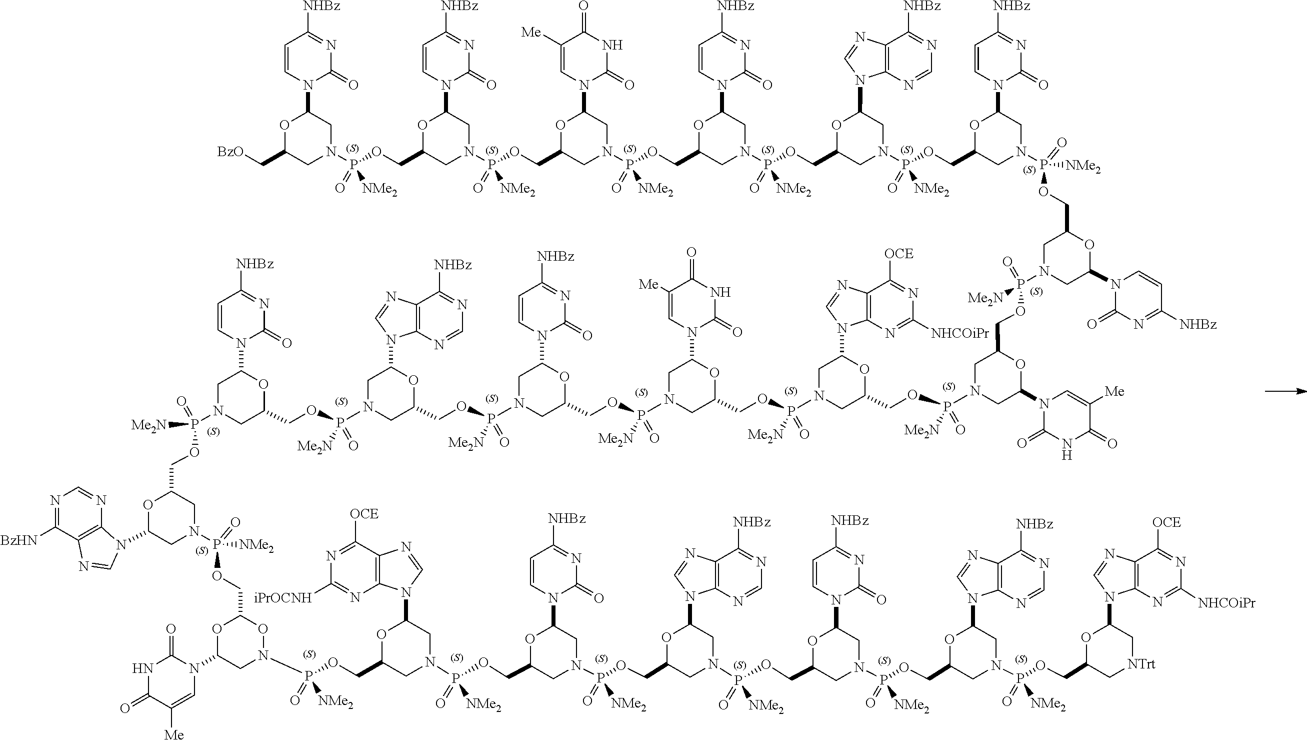

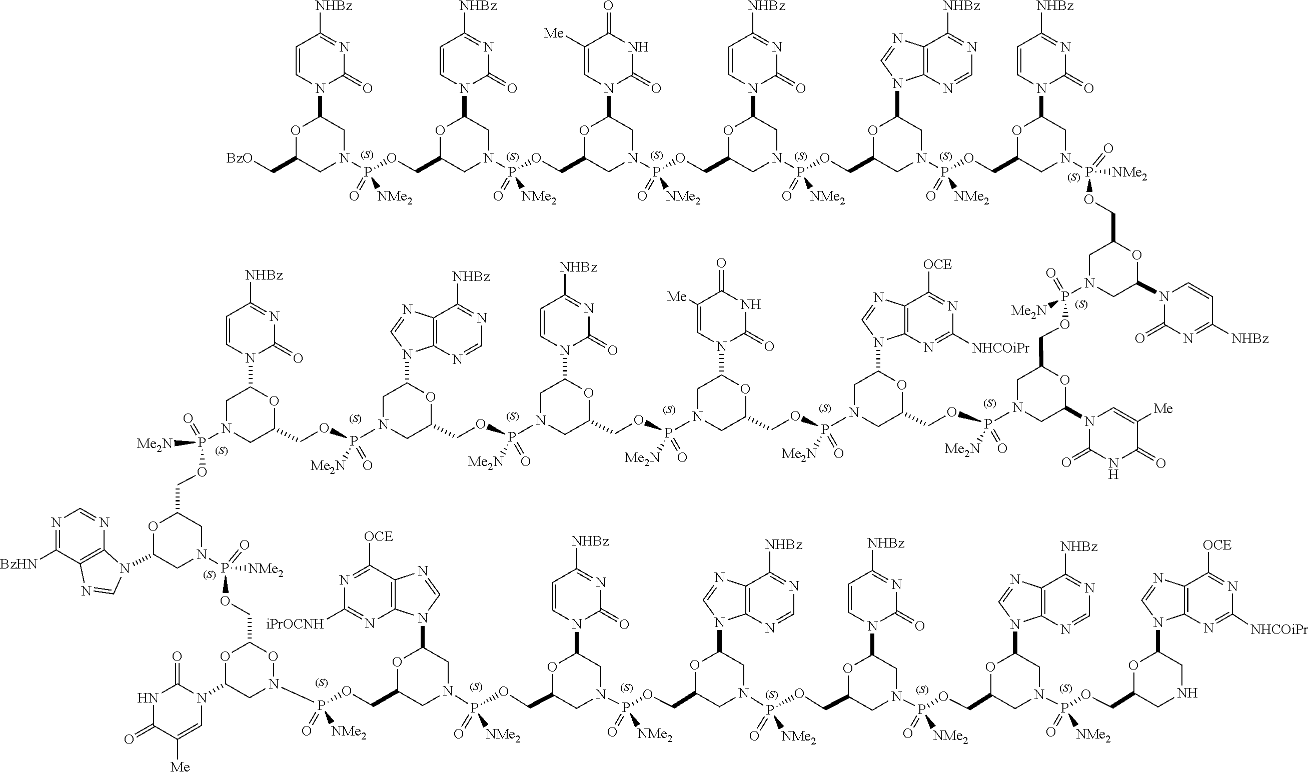

Using General Procedure B: reaction of trityl protected 21-mer (800 mg) afforded free base 21-mer (756 mg).

Coupling to 22-mer:

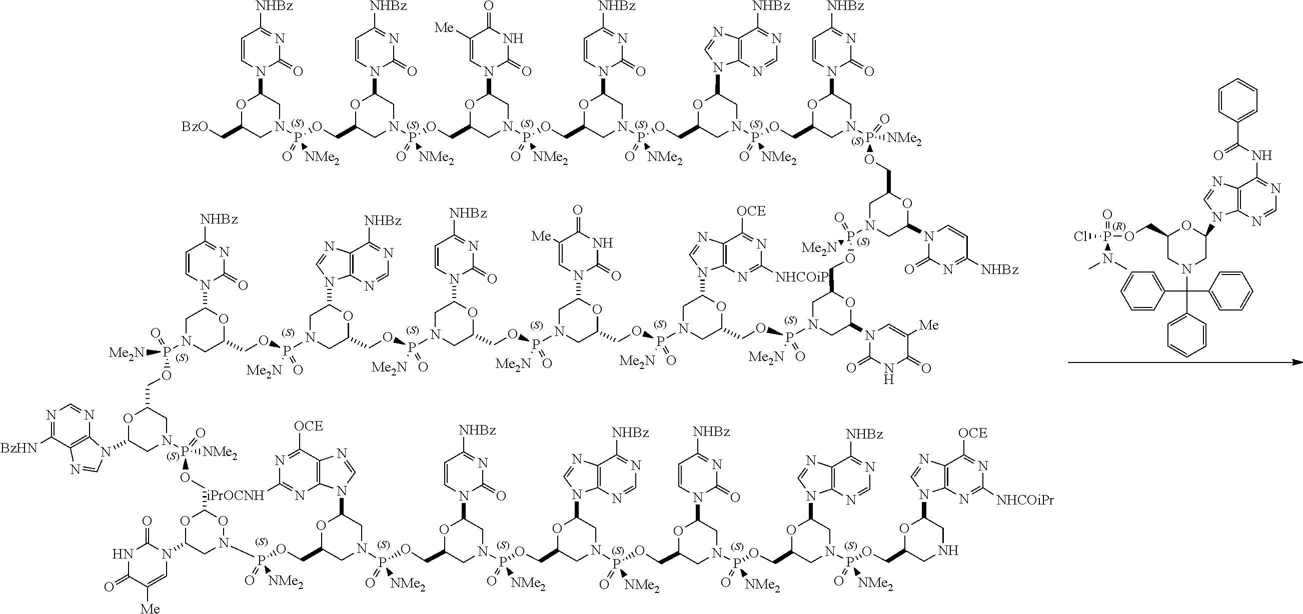

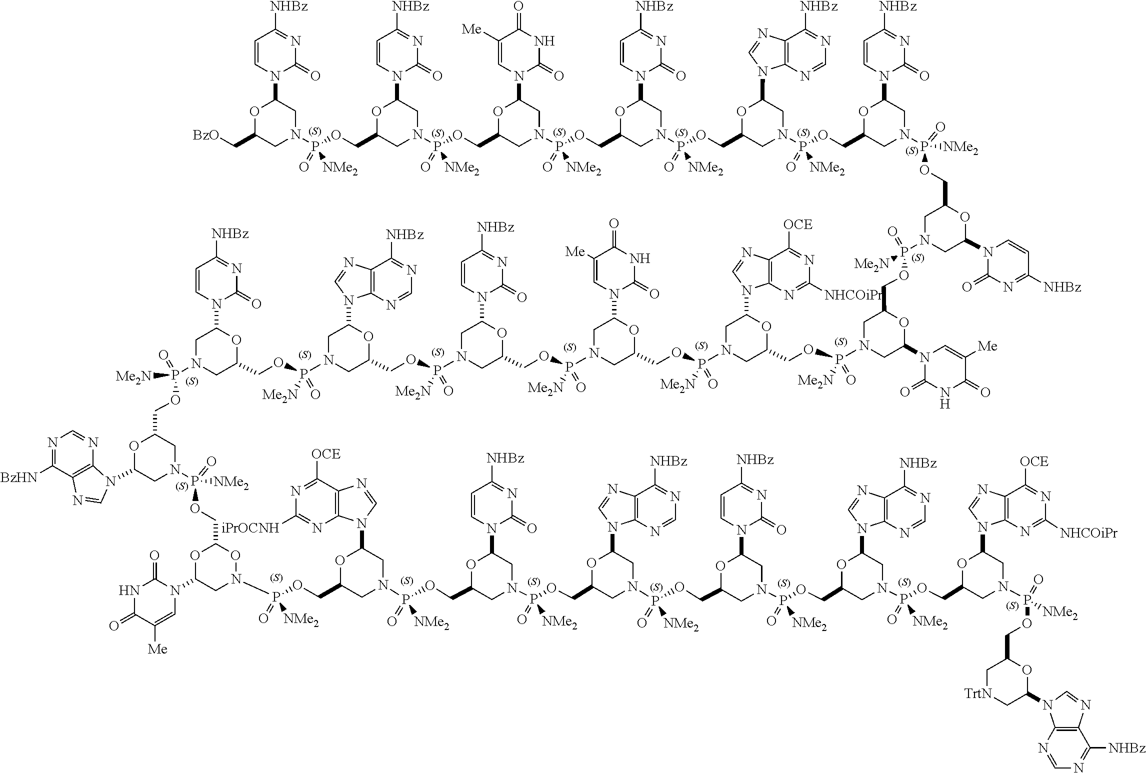

Using General Procedure A: reaction of 21-mer (753 mg) with ((2S,6R)-6-(6-benzamido-9H-purin-9-yl)-4-tritylmorpholin-2-yl)methyl (R)-dimethylphosphoramidochloridate (137 mg) afforded 22-mer (806 mg).

Deprotection of 22-mer:

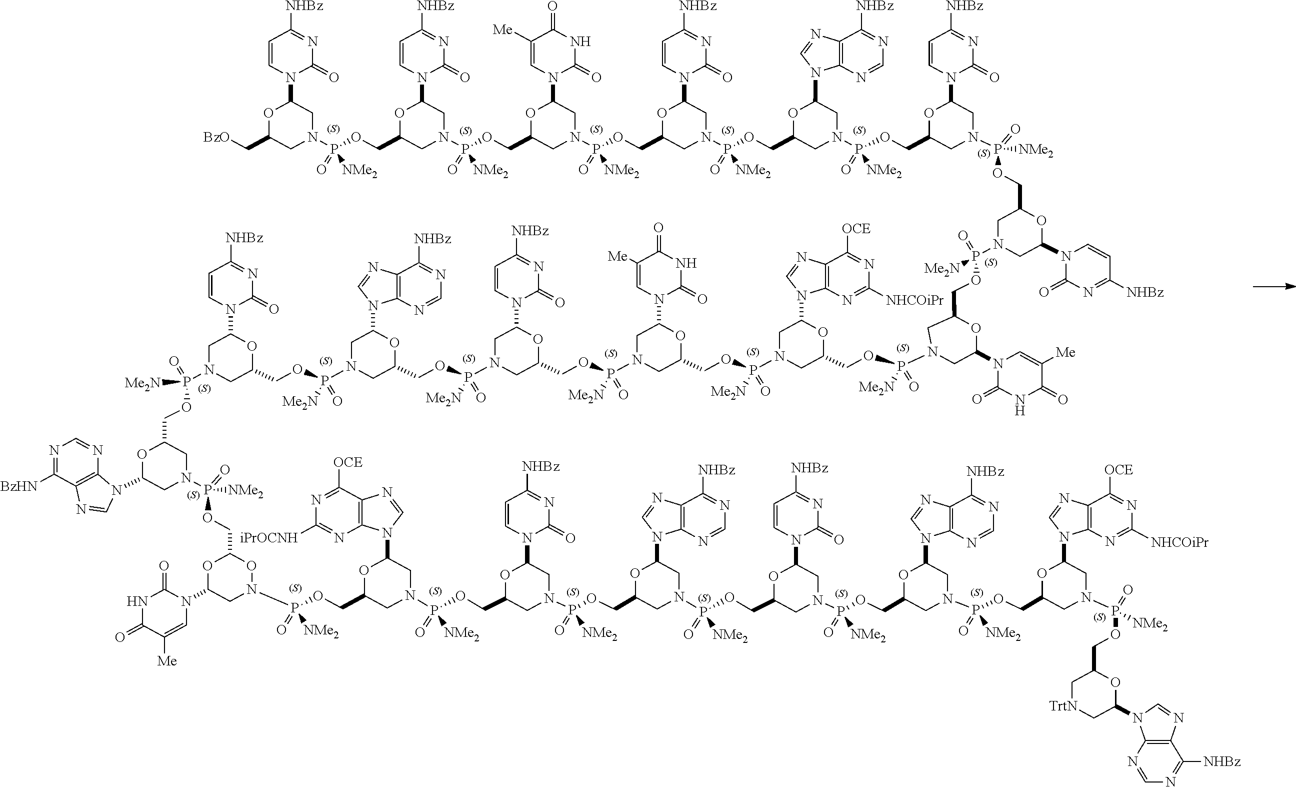

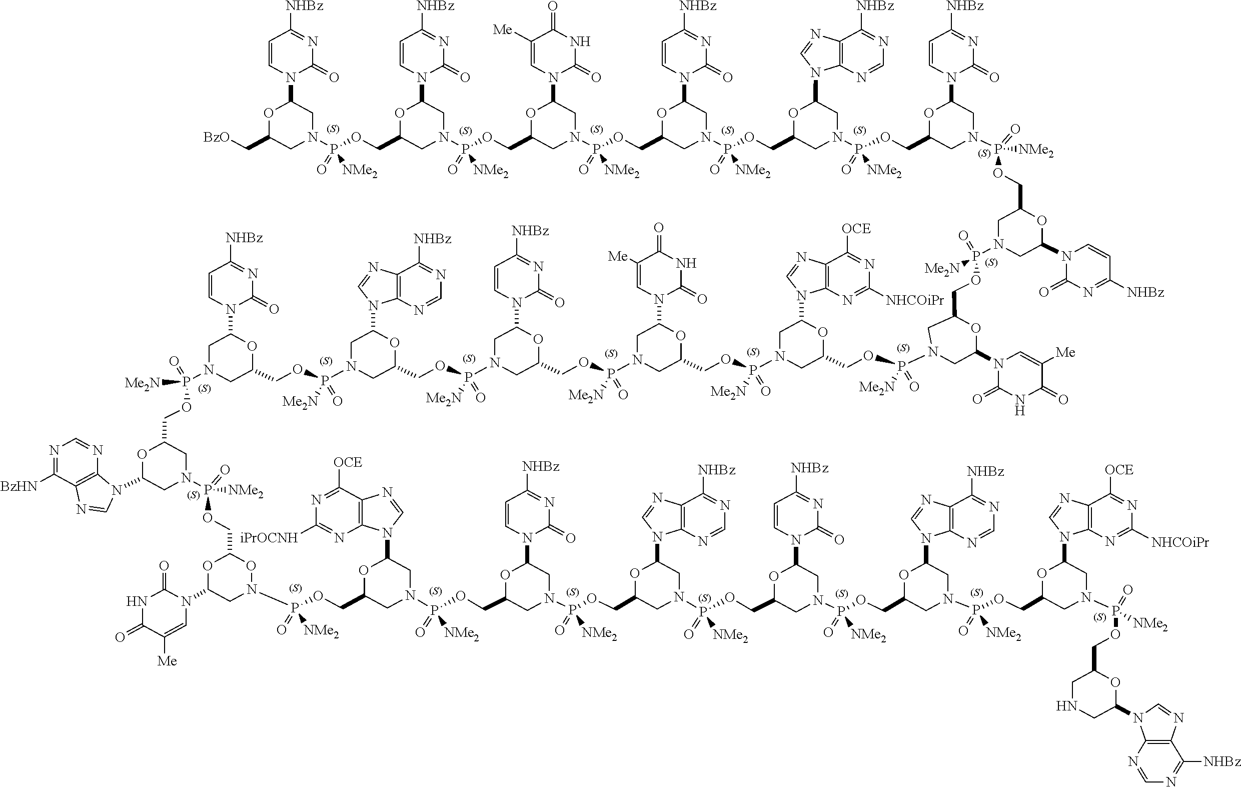

Using General Procedure B: reaction of trityl protected 22-mer (806 mg) afforded free base 22-mer (785 mg).

Coupling to 23-mer:

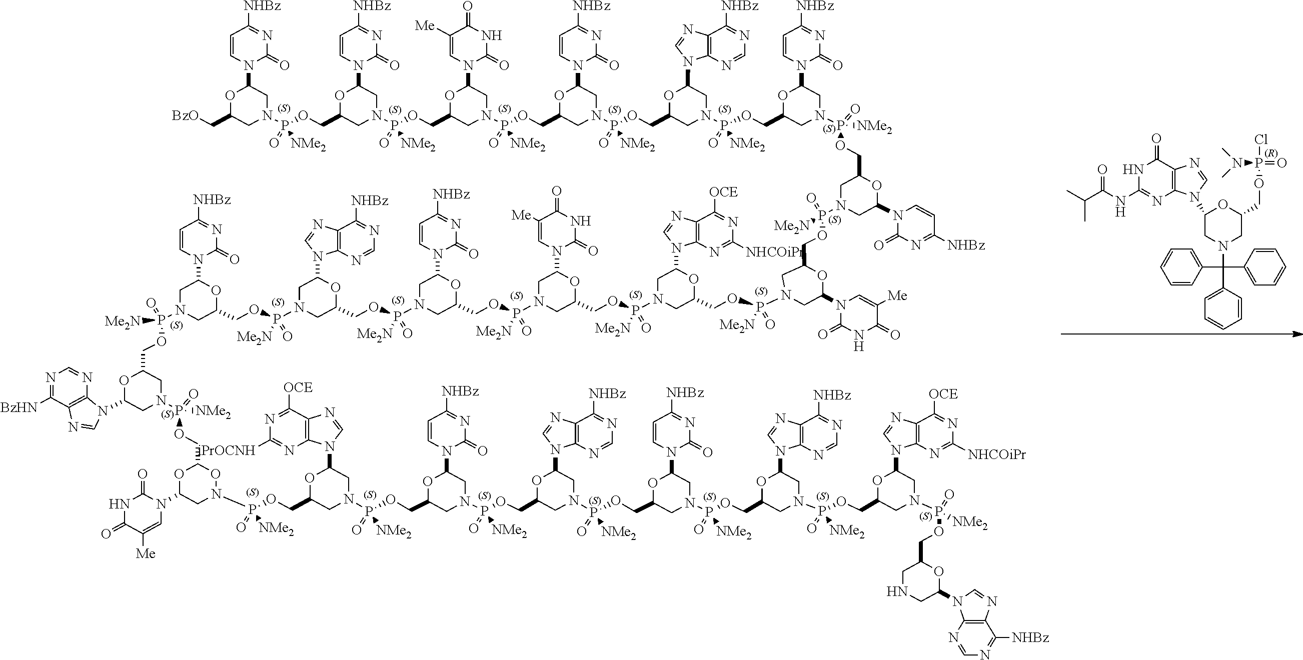

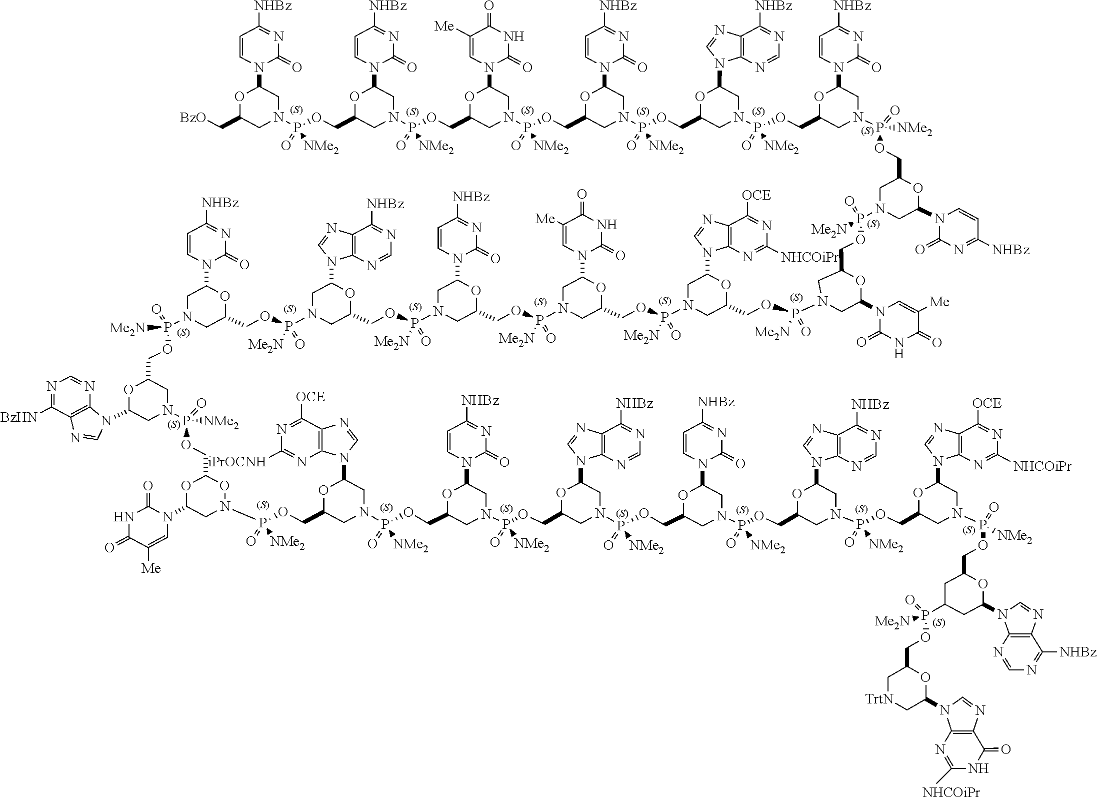

Using General Procedure A: reaction of 22-mer (780 mg) with ((2S,6R)-6-(2-isobutyramido-6-oxo-1,6-dihydro-9H-purin-9-yl)-4-tritylmorpholin-2-yl)methyl (R)-dimethylphosphoramidochloridate (161 mg) afforded 23-mer (837 mg).

Deprotection of 23-mer:

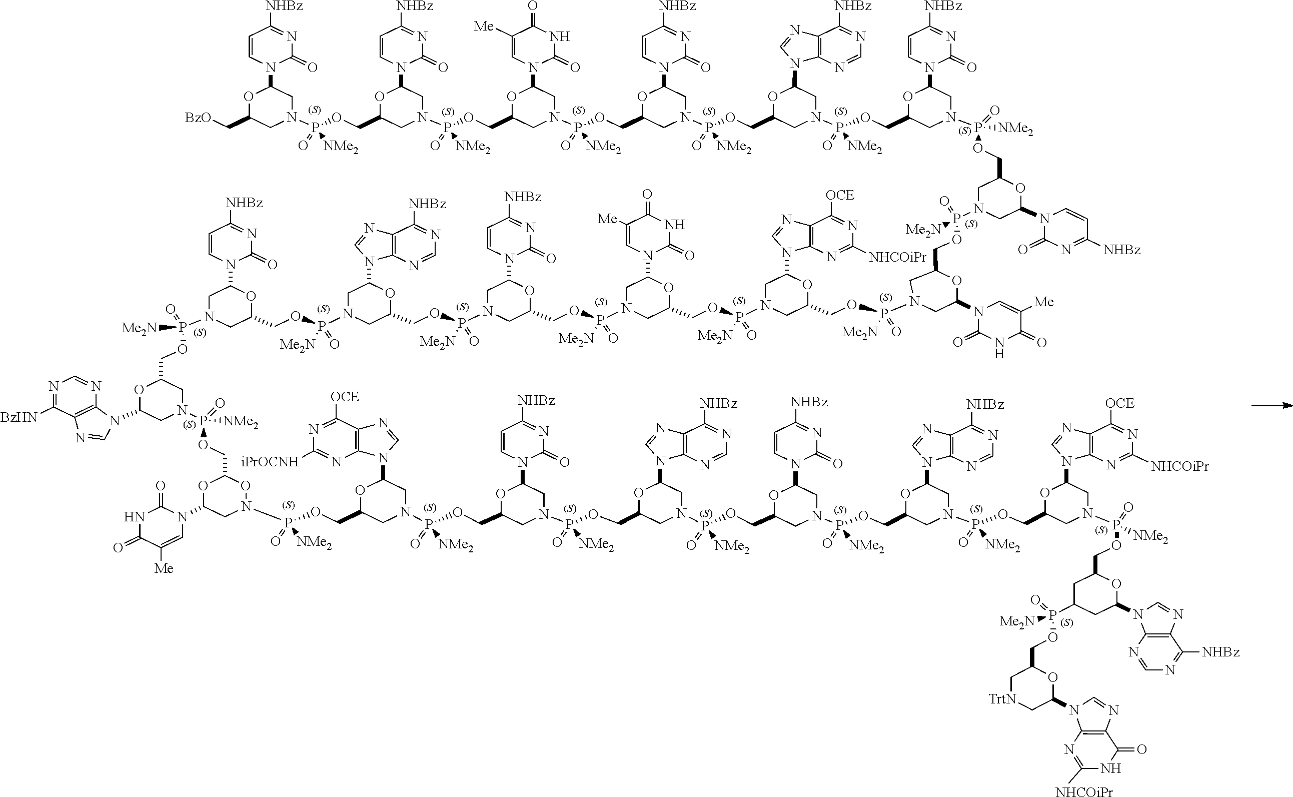

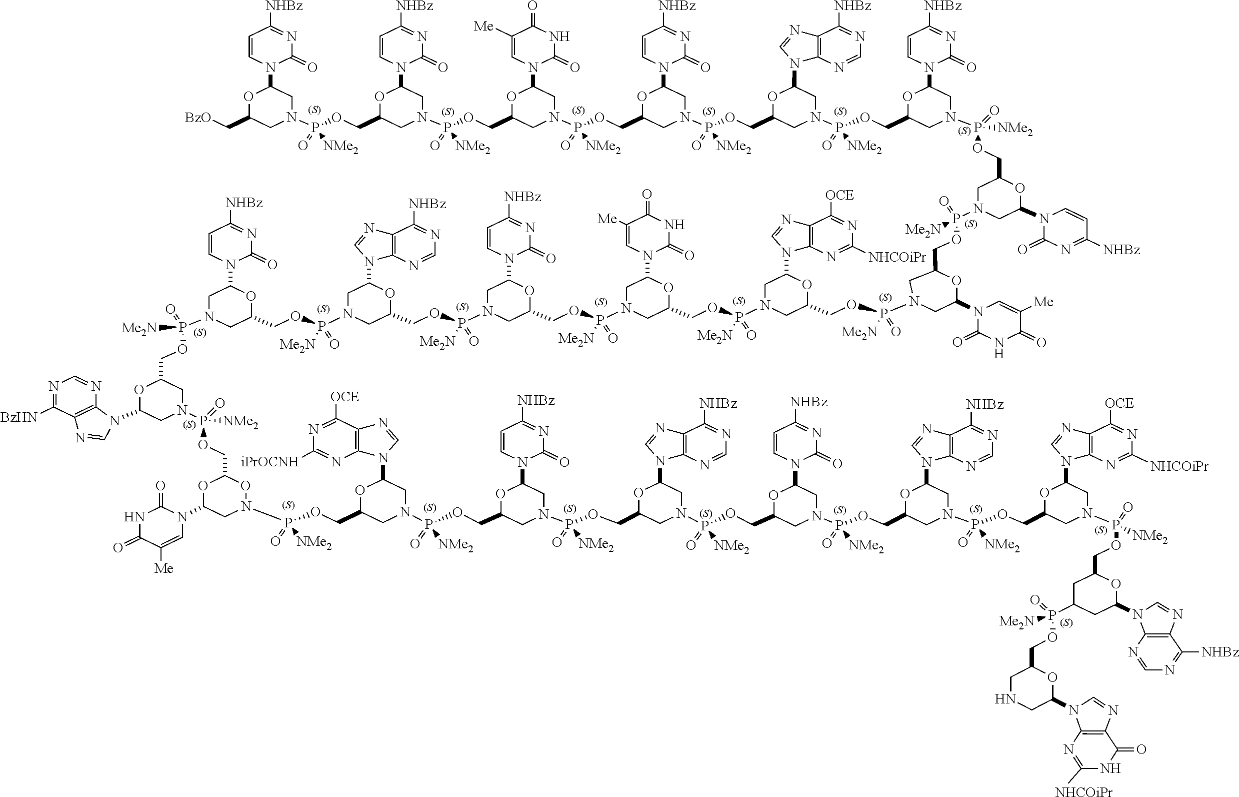

Using General Procedure B: reaction of trityl protected 23-mer (837 mg) afforded free base 23-mer (830 mg).

Coupling to 24-mer:

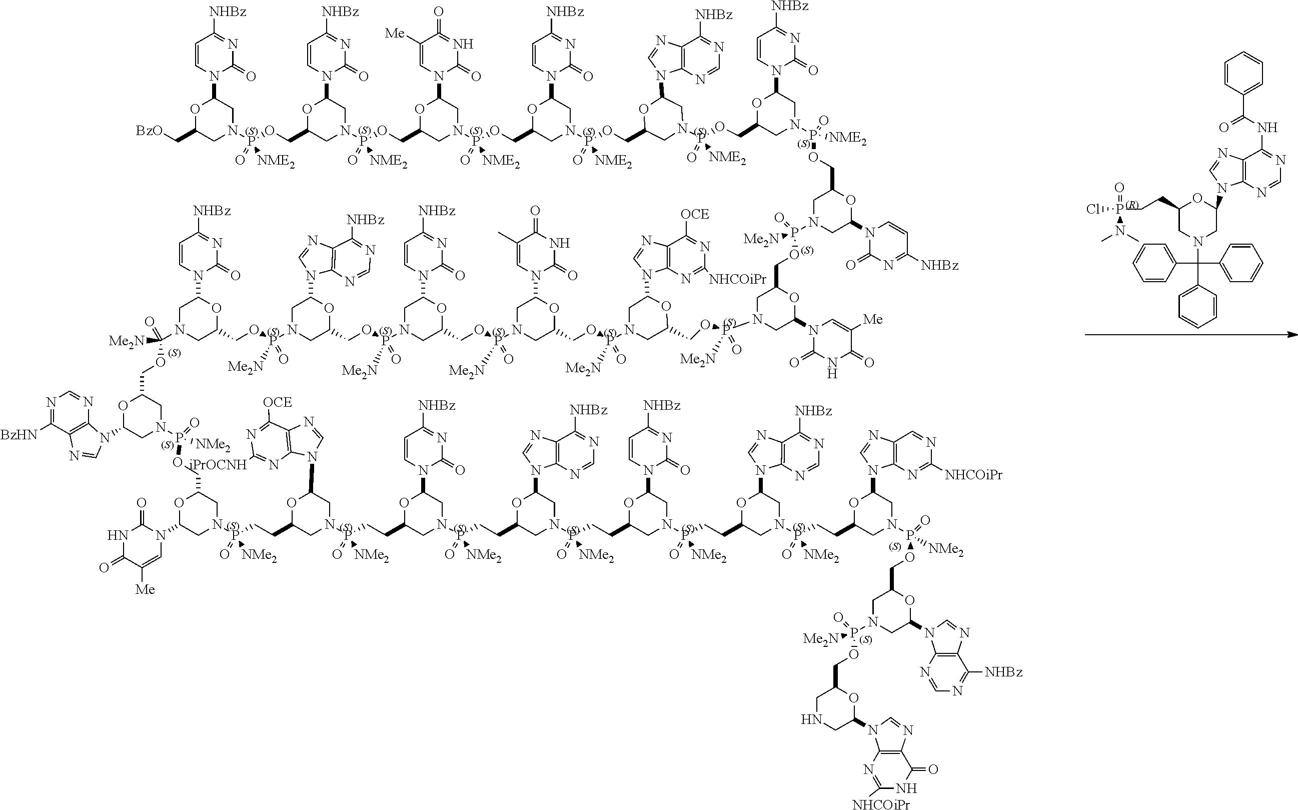

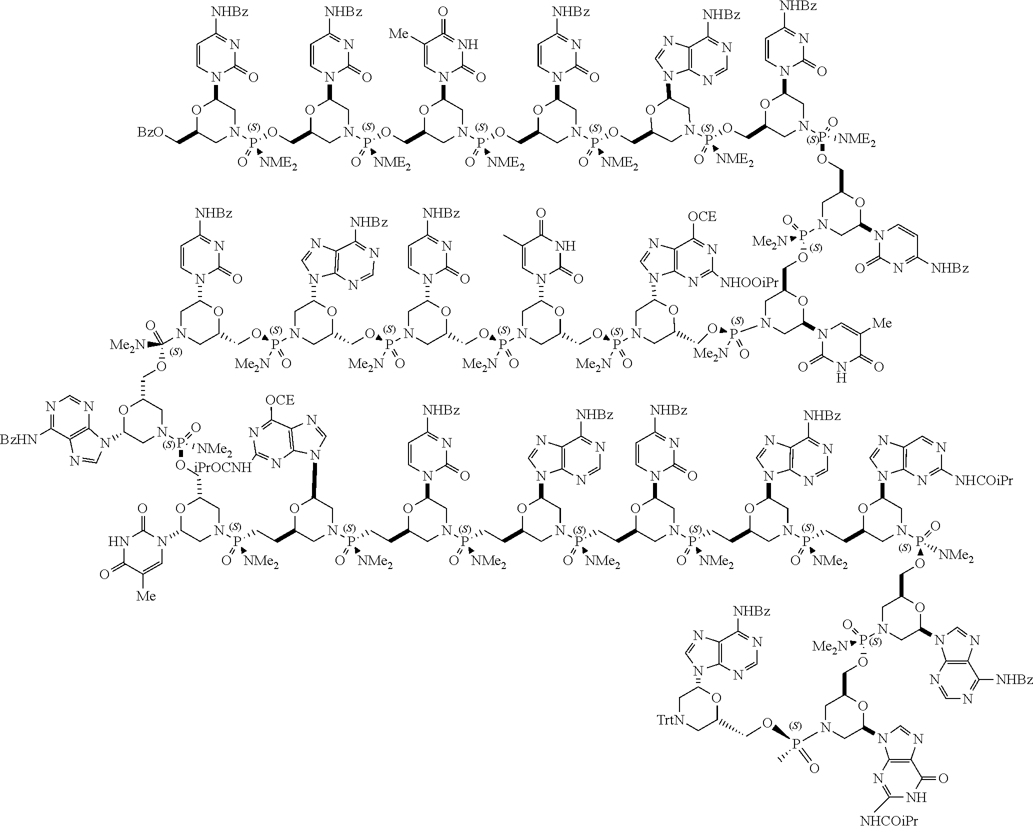

Using General Procedure A: reaction of 23-mer (830 mg) with ((2S,6R)-6-(6-benzamido-9H-purin-9-yl)-4-tritylmorpholin-2-yl)methyl (R)-dimethylphosphoramidochloridate (177 mg) afforded 24-mer (800 mg).

Deprotection of 24-mer:

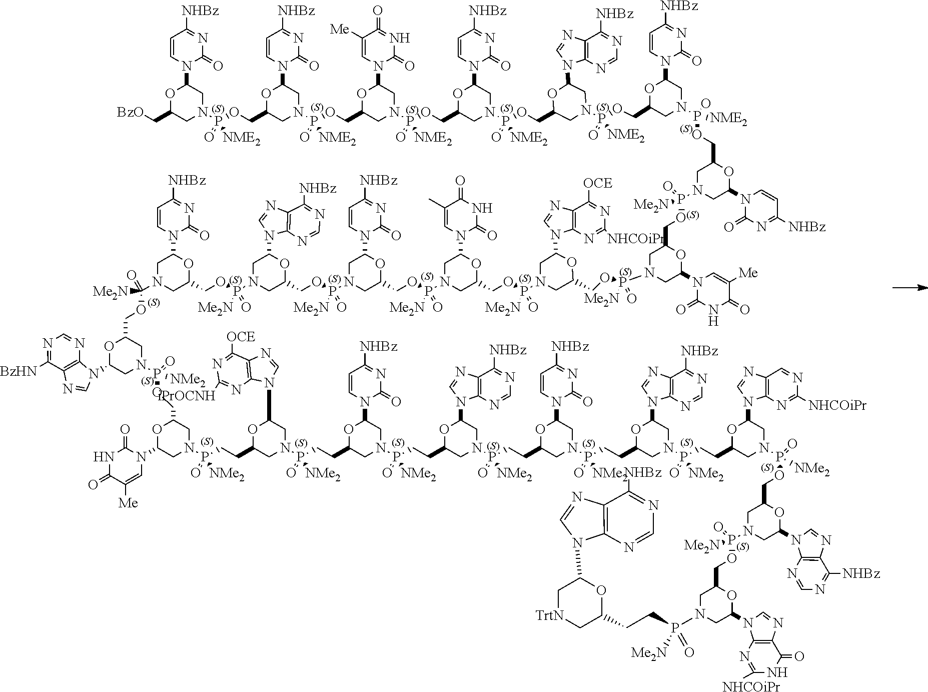

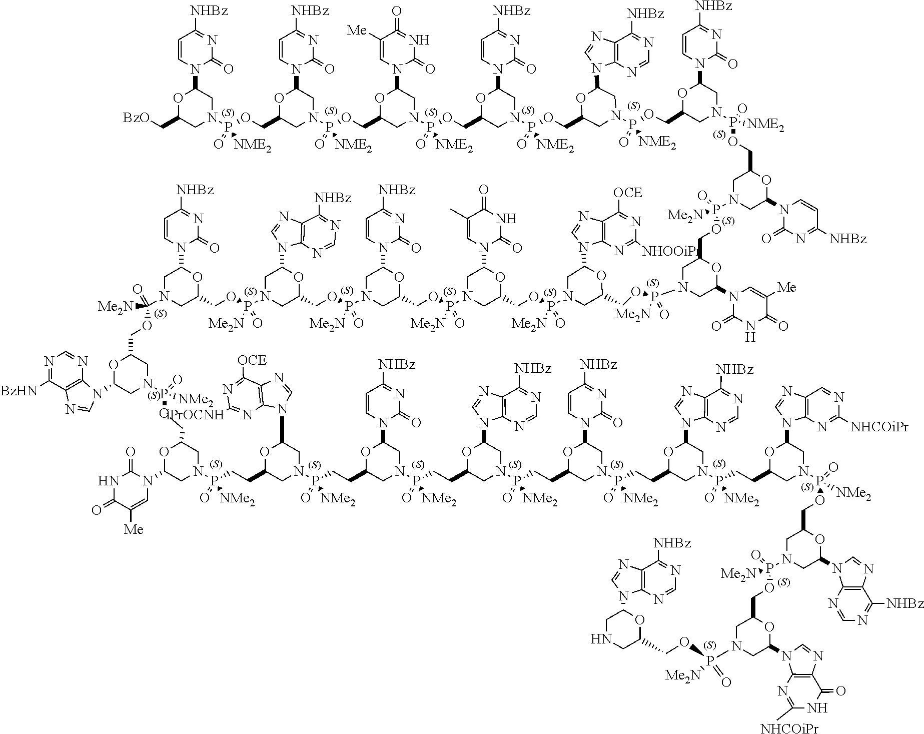

Using General Procedure B: reaction of trityl protected 24-mer (800 mg) afforded free base 24-mer (793 mg).

Coupling to 25-mer:

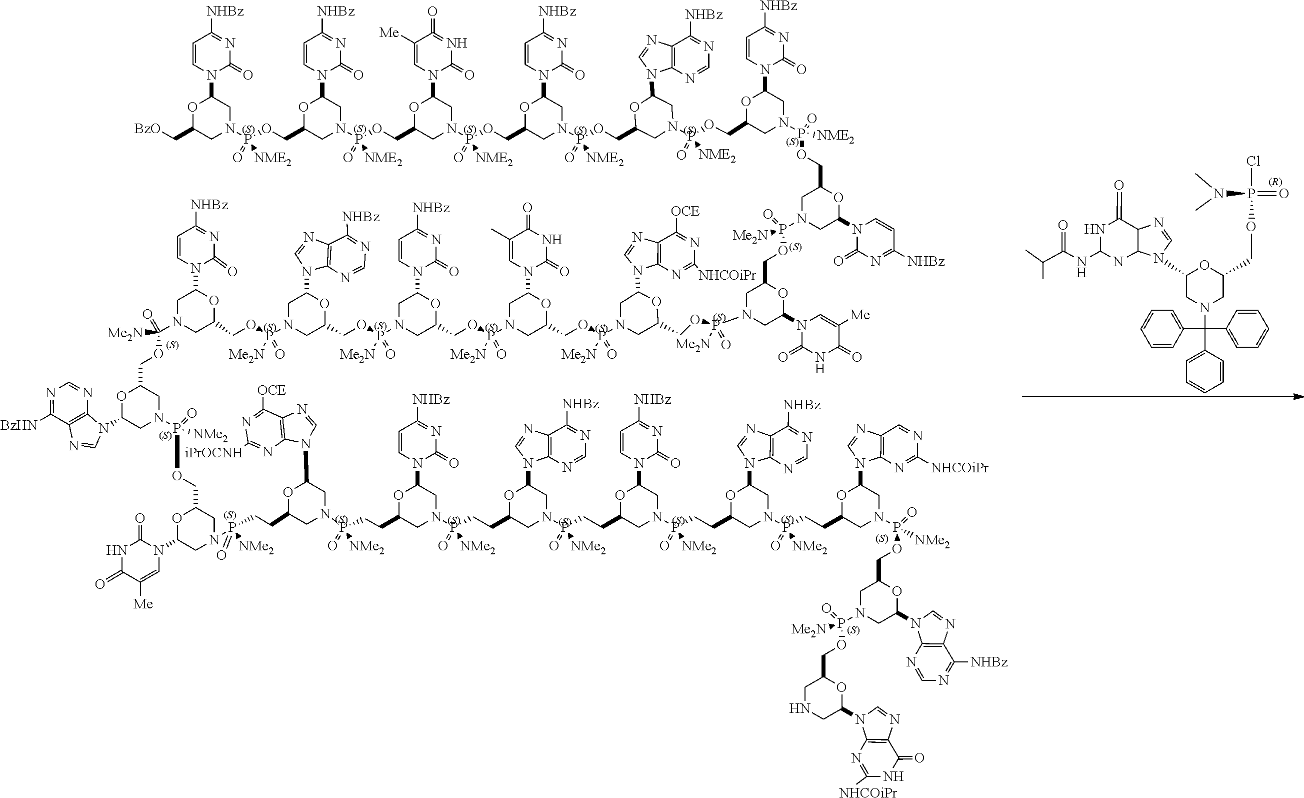

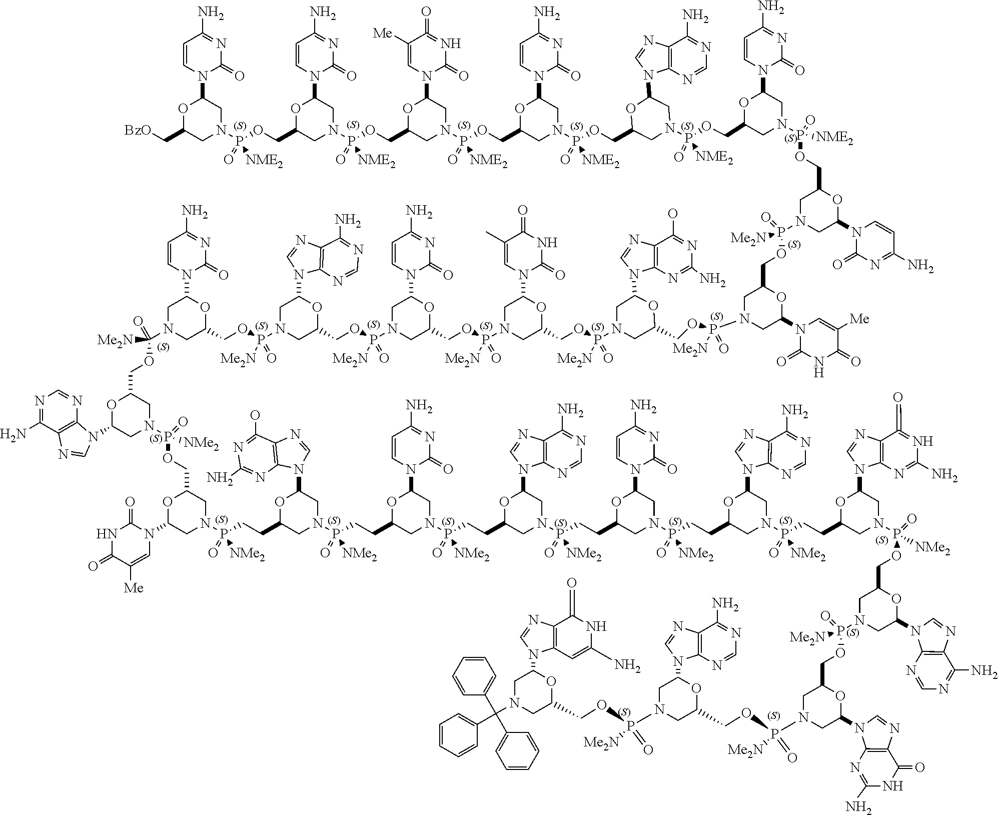

Using General Procedure A: reaction of 24-mer (793 mg) with ((2S,6R)-6-(2-isobutyramido-6-oxo-1,6-dihydro-9H-purin-9-yl)-4-tritylmorpholin-2-yl)methyl (R)-dimethylphosphoramidochloridate (150 mg) afforded 25-mer (818 mg). MS (ESI) m/z: Calcd for C₄₅₆H₅₇₁N₁₄₇O₁₁₈P₂₄: [M+7H/7]⁺ 1535.53. Found: 1535.56.

Base Deprotection of 25-mer:

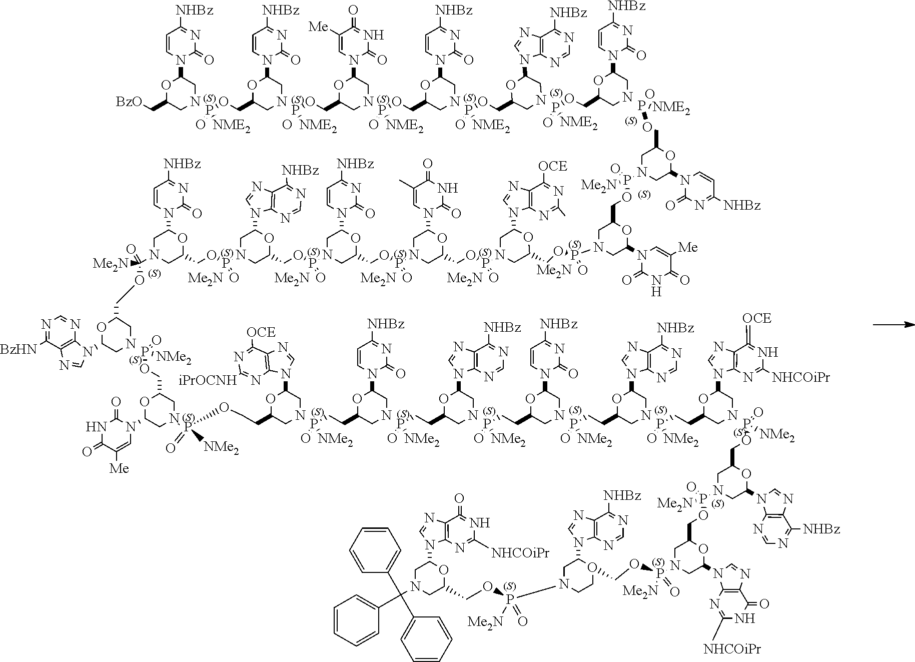

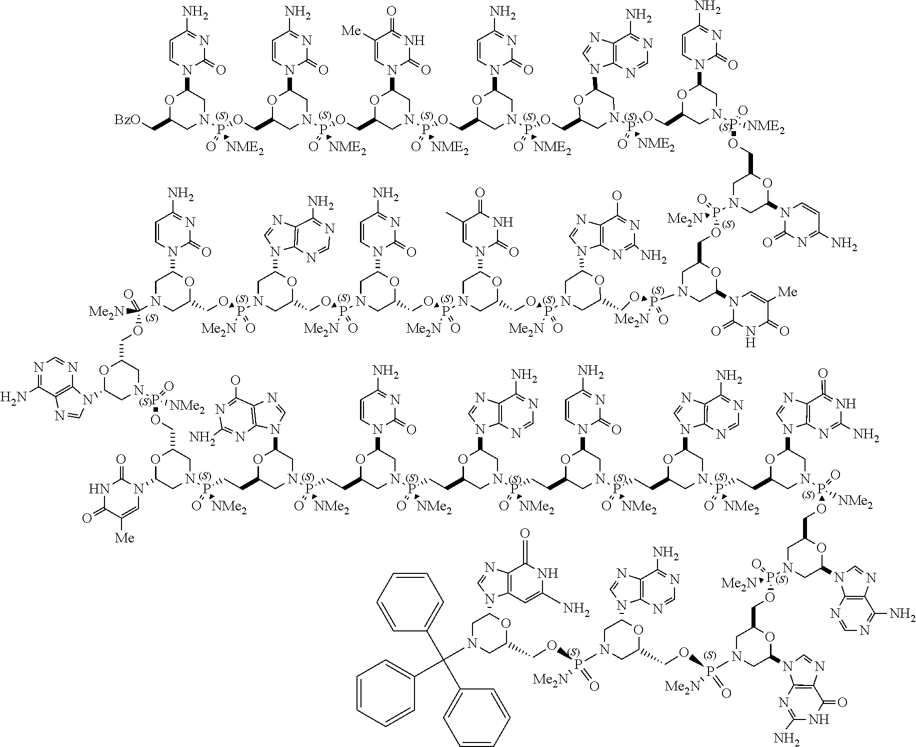

To a 100 ml flask with 25-mer (710 mg) were added methanol (19.5 mL) and 28% aqueous ammonium hydroxide (19.5 ml-) under nitrogen atmosphere. The reaction mixture was stirred at 50 °C for 2 days affording a clear solution. The solution was then evaporated under vacuum to a volume of ca 20 mL at 35 °C. The slightly cloudy mixture was filtered through a plastic fritted funnel and rinsed with ca 5-10 mL of water for a total adjusted volume of 25 mL. The resulting solution was used for purification by reverse phase HPLC using the conditions below. The desired peak fractions were evaporated to afford a total of 148 mg deprotected Trityl-on 25-mer as a white solid. MS (ESI) m/z: Calcd for C₃₀₈H₄₆₄N₁₄₄O₉₆P₂₄: [M+1H]⁺ 8462.97. Found: 8463.00 (deconvoluted HRMS spectrum).

### Purification Conditions for Trityl-Protected PMO:

| Column: | Waters Xbridge prep C18 5 um OBD 19 × | | | |
|---|---|---|---|---|
| | 100 mm (Part Number: 186002978) | | | |
| Instrument: | Waters Prep HPLC | | | |
| Mobile phase A: | 10 mM NH4HCO3, pH 10 (with NH4OH) | | | |
| Mobile phase B: | 15% of Mobile Phase A and 85% acetonitrile | | | |
| Column Temperature: | Unregulated | | | |
| | TIME (min) | A % | B % | |
| Gradient: | 0 | 80 | 20 | Initial |
| | 12 | 65 | 35 | Elution Gradient |
| | 13 | 0 | 100 | |
| | 14 | 0 | 100 | |
| | 15 | 80 | 20 | Reset Conditions |

| | |
|---|---|
| Flow Rate (mL/min): | 40 |
| Wavelength: | DAD + 260 nm + timed collection |

Trityl Deprotection of 25-mer:

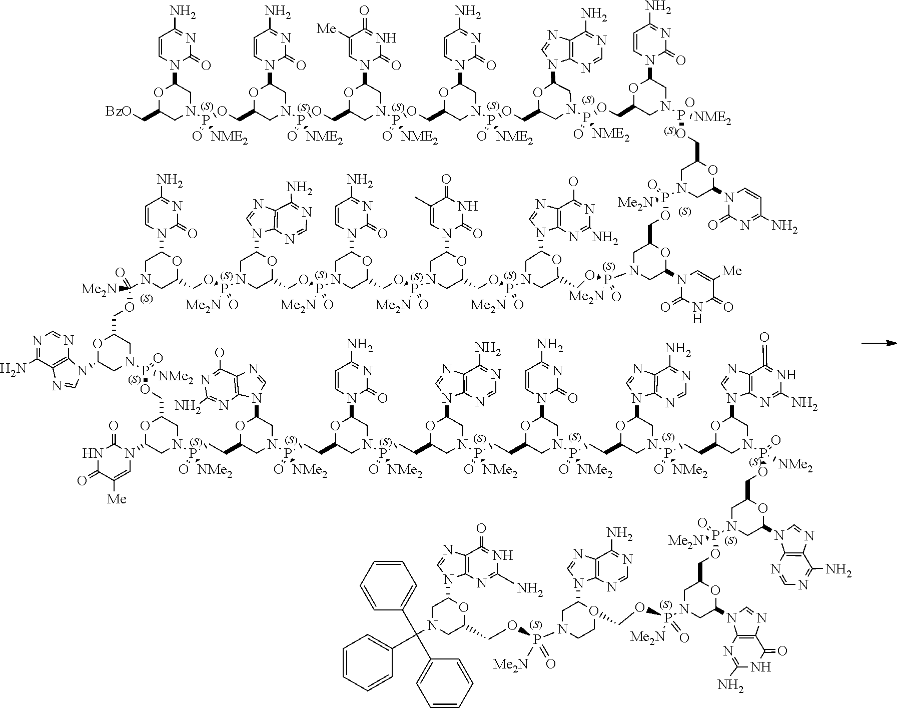

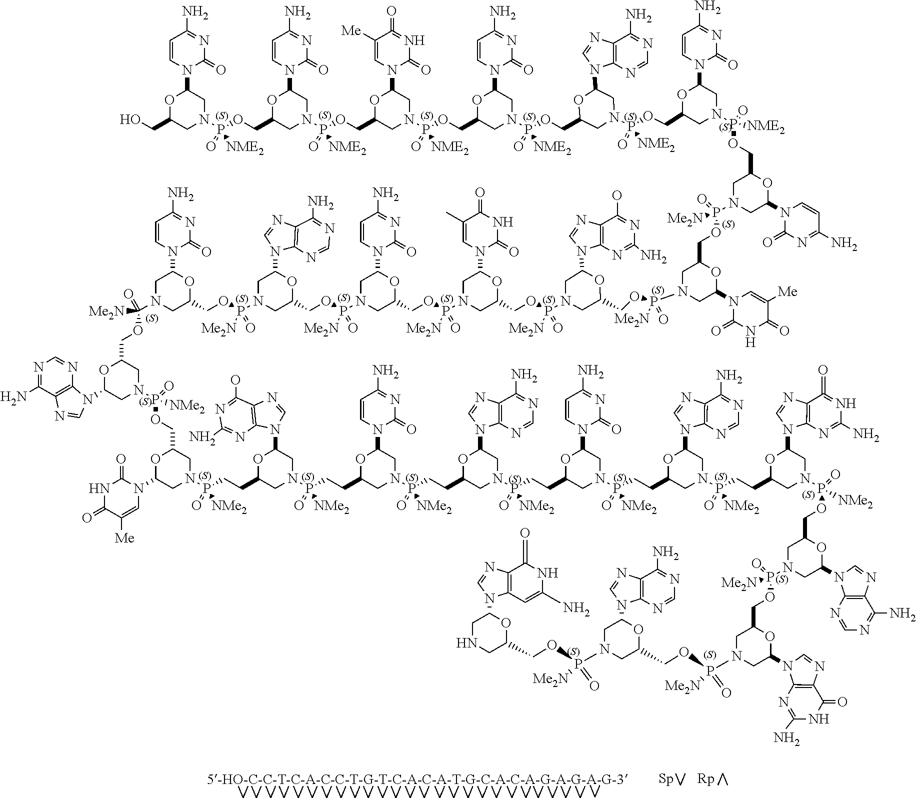

To a vial with trityl protected 25-mer (3.7 mg) was added 0.1 M phosphoric acid (250 μL). The vial was agitated at rt for 4 h when the reaction was deemed completed (two consecutive checks by UPLC MS shows starting material peak was converted to a earlier eluting peak). Added 0.1M ammonium hydroxide (250 μL) and filtered through a syringe filter of 0.2 μM. The filter was rinsed with 0.4 mL of water and collected into a vial. The sample was purified by reverse phase HPLC using the method in the below table. The desired fractions were combined and evaporated under vacuum, then lyophilized to afford the desired product 1.2 mg of 25-mer PMO (PMO-302). MS (ESI) m/z: Calcd for C₂₈₉H₄₅₀N₁₄₄O₉₆P₂₄: [M+1H]⁺ 8220.86. Found: 8220.87 (deconvoluted HRMS spectrum).

### Purification Conditions for Fully-Deprotected PMO:

| Column: | Waters Xbridge prep C18 5 um OBD 19 × | | | |
|---|---|---|---|---|
| | 100 mm (Part Number: 186002978) | | | |
| Instrument: | Waters Prep HPLC | | | |
| Mobile phase A: | Water + 0.1% Ammonium Hydroxide | | | |
| Mobile phase B: | Acetonitrile + 0.1% Ammonium Hydroxide | | | |
| Column Temperature: | Unregulated | | | |
| | TIME (min) | A % | B % | |
| Gradient: | 0 | 95 | 5 | Initial |
| | 1.1 | 95 | 5 | Elution Gradient |
| | 11.1 | 80 | 20 | |
| | 12.1 | 5 | 95 | |
| | 13.1 | 5 | 95 | |
| | 13.2 | 95 | 5 | Reset Conditions |
| | 15 | 95 | 5 | |

| | |
|---|---|
| Flow Rate (mL/min): | 40 |
| Wavelength: | DAD + 260 nm + timed collection |

### Example 4: Additional Exemplary PMO-ASOs

PMO oligonucleotides were designed for screening. The designed oligonucleotides were made by GeneTools LLC (website: www.gene-tools.com) by solid-phase method. Table 13 below lists synthesized PMO oligonucleotides with their deconvoluted MS data. These PMO oligonucleotides are complementary to a section of SEQ ID NO:1 showing increased Exon-2 skipping activity. In particular, PMO-221 through PMO 240, PMO-324, PMO-424, PMO-402 and PMO-502 are complementary to Region 1; and PMO-241 through PMO-244 are complementary to Region 2. All PMO oligonucleotides listed in Table 13 below contain a phosphorodiamidate-attached sarcosine (Sar) linker at the 5′ end. All PMO oligonucleotides listed in Table 13 below were synthesized with unmodified cytosine PMO nucleotide. All PMO oligonucleotides listed in Table 13 below have stereorandom internucleotide linkages, and thus are called stereorandom PMO oligonucleotides. The structure of PMO-224 is as follows:

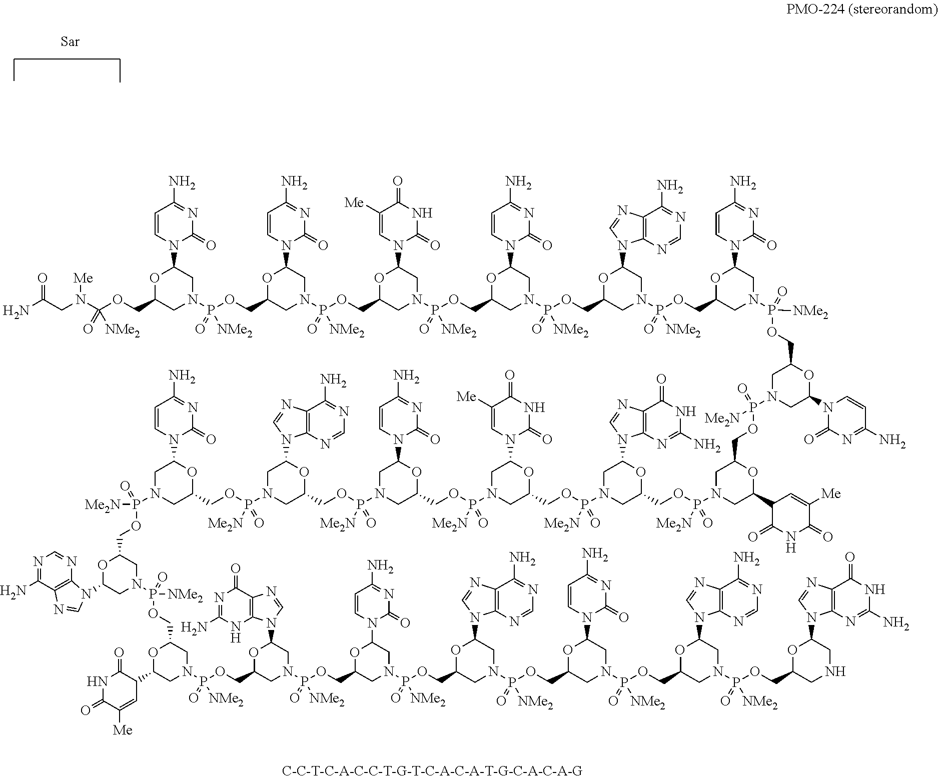

TABLE 13

| | | | MW | |
|---|---|---|---|---|
| | | | (with | |
| | | SEQ. ID | Sarcosine | MS |
| ASO # | Seq. | NO: | linker) | observed |
| PMO-221 | 5′ -CCTCACCTGTCACATGCACAGAG-3′ | 221 | 7703.47 | 7709.4 |
| PMO-222 | 5′ -TCACCTGTCACATGCACAGAGAG-3′ | 222 | 7767.52 | 7773.6 |
| PMO-223 | 5′ -CTCACCTGTCACATGCACAGAGA-3′ | 223 | 7727.49 | 7733.6 |
| PMO-224 | 5′ -CCTCACCTGTCACATGCACAG-3′ | 224 | 7008.88 | 7013.9 |
| PMO-225 | 5′ -ACCTGTCACATGCACAGAGAG-3′ | 225 | 7121.98 | 7127.1 |
| PMO-226 | 5′ -TCACCTGTCACATGCACAGAG-3′ | 226 | 7072.93 | 7077.9 |
| PMO-227 | 5′ -TCACCTGTCACATGCACAGAGAGCT-3′ | 227 | 8413.07 | 8418.5 |
| PMO-228 | 5′ -CCTGTGCCTCACCTGTCACATGCAC-3′ | 228 | 8315.97 | 8320.0 |
| PMO-229 | 5′ -GTGCCTCACCTGTCACATGCACAGA-3′ | 229 | 8389.04 | 8393.6 |
| PMO-230 | 5′ -TGCCTCACCTGTCACATGCACAGAG-3′ | 230 | 8389.04 | 8393.7 |
| PMO-231 | 5′ -CTCACCTGTCACATGCACAGAGAGC-3′ | 231 | 8398.06 | 8386.1 |
| PMO-232 | 5′ -CACCTGTCACATGCACAGAGAGCTG-3′ | 232 | 8438.08 | 8441.8 |
| PMO-233 | 5′ -ACCTGTCACATGCACAGAGAGCTGG-3′ | 233 | 8478.11 | 8483.7 |
| PMO-234 | 5′ -CTGTCACATGCACAGAGAGCTGGGG-3′ | 234 | 8534.14 | 8539.6 |
| PMO-235 | 5′ -CCTGTCACATGCACAGAGAGCTG-3′ | 235 | 7783.52 | 7789.6 |
| PMO-236 | 5′ -TGTCACATGCACAGAGAGCTGGG-3′ | 236 | 7863.58 | 7868.6 |
| PMO-237 | 5′ -CTGTCACATGCACAGAGAGCTGG-3′ | 237 | 7823.55 | 7828.0 |
| PMO-238 | 5′ -TGTCACATGCACAGAGAGCTGG-3′ | 238 | 7508.28 | 7513.6 |
| PMO-239 | 5′ -TCACATGCACAGAGAGCTGGG-3′ | 239 | 7178 | 7183.4 |
| PMO-240 | 5′ -TGTCACATGCACAGAGAGCTG-3′ | 240 | 7152.99 | 7157.2 |
| PMO-241 | 5′ -CTGTATTTGGTACTTCCTCTCTCCA-3′ | 241 | 8326.97 | 8332.8 |
| PMO-242 | 5′ -TGTATTTGGTACTTCCTCTCTCCAT-3′ | 242 | 8341.98 | 8347.2 |
| PMO-243 | 5′ -GTATTTGGTACTTCCTCTCTCCATC-3′ | 243 | 8326.97 | 8332.8 |
| PMO-244 | 5′ -TATTTGGTACTTCCTCTCTCCATCC-3′ | 244 | 8286.94 | 8292.3 |

### Example 5: Synthesis of PMO Oligionucleotides with Stereopure Internucleotide Linkages and 5′-Sarcosine Linkers

TABLE 14
Stereopure PMO oligonucleotides

| ASO name | Sequence |
|---|---|
| PMO-324 (all Rp | 5′ -CCTCACCTGTCACATGCACAG-3′ |
| internucleotide | |
| linkages) | |
| PMO-424 (all Sp | 5′-CCTCACCTGTCACATGCACAG-3′ |
| internucleotide | |
| linkages) | |
| PMO-402 (all Rp | 5′-CCTCACCTGTCACATGCACAGAGAG-3′ |
| internucleotide | |
| linkages) | |
| PMO-502 (all Sp | 5′-CCTCACCTGTCACATGCACAGAGAG-3′ |
| internucleotide | |
| linkages) | |

### Solution Phase Synthesis of Stereopure PMO Oligonucleotides:

Solution phase synthesis of 5′-sarcosine capped stereopure oligonucleotides in Table 14 was conducted using similar methods to those methods described in Example 3 (using general Procedures A and B) with the exception of Step 1 which started with coupling sarcosine benzyl ester to a stereopure cytosine dimethylphosphoramidochloridate. Briefly, the synthesis includes iterative steps of deprotection/free basing/coupling as depicted here for all Sp internucleotide linkages):

### Stop 1 for Synthesis of PMO-424 and PMO-602 by Solution Phase Synthesis:

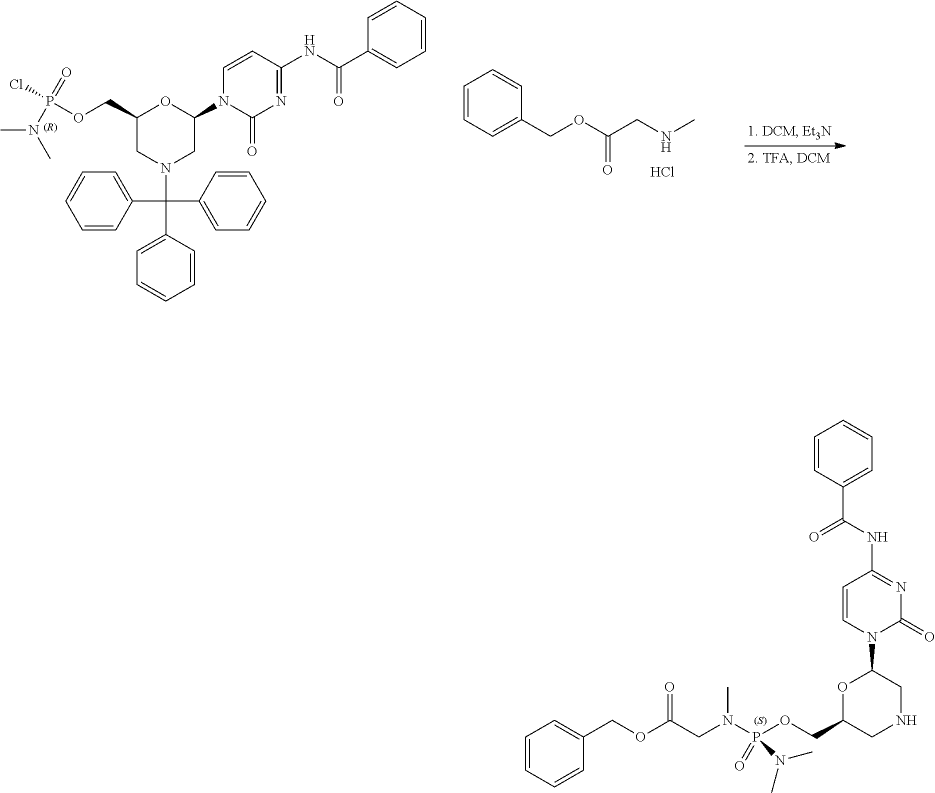

### General Scheme for Elongation Process

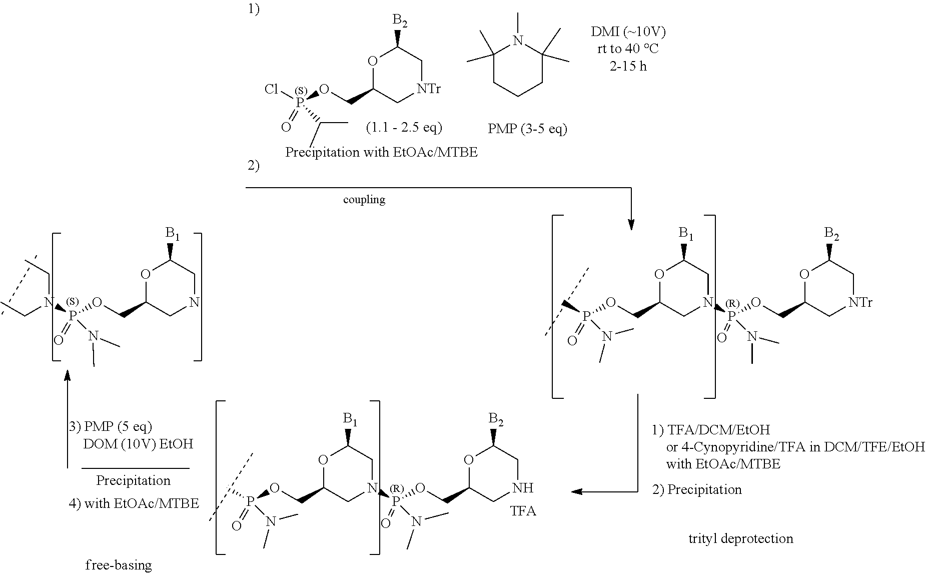

General scheme for synthesis of PMO-424 and PMO-502 by solution phase.

Briefly, the synthesis includes iterative steps of deprotection/free basing/coupling as depicted here for all Rp internucleotide linkages):

### Step 1 for Synthesis of PMO-324 and PMO-402 by Solution Phase Synthesis:

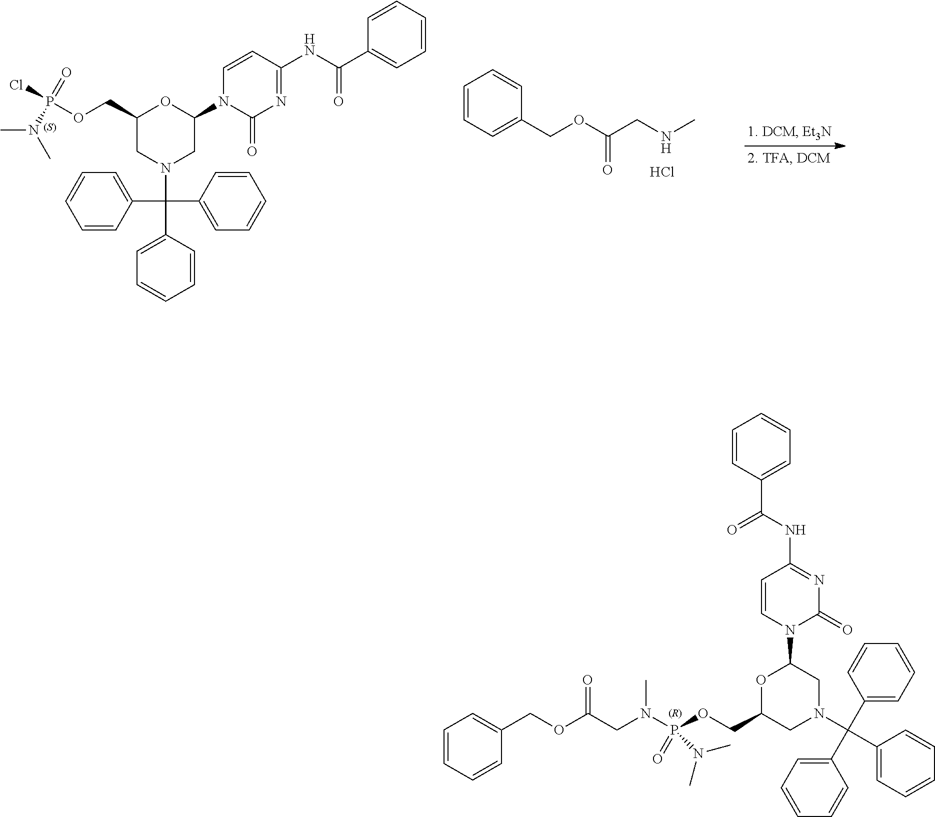

### General Scheme for Elongation Process

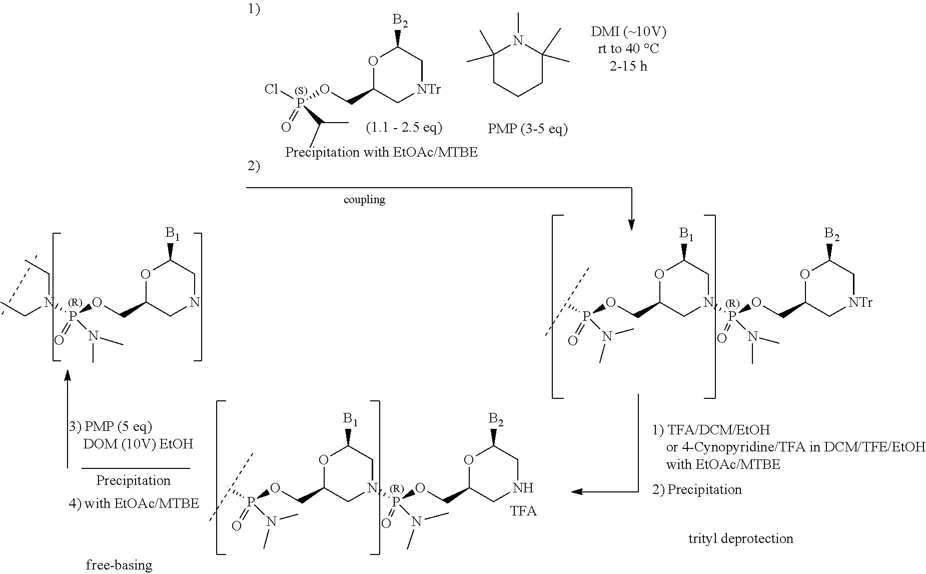

### General Scheme for Synthesis of PMO-324 and PMO-402 by Solution Phase.

At the conclusion of each individual step, precipitation of the oligonucleotide was achieved by addition of non-polar solvent such as MTBE and/or EtOAc. In elongation steps up to sixmer, purification of the 3′-N-trityl protected oligonucleotide was conducted by silica gel chromatography using DCM/MeOH as eluent.

Upon reaching the desired oligonucleotide length (21-mer for PMO-324, PMO-424, and 25-mer for PMO-402 and PMO-502), the 3′-N-trityl protected sequence was subjected to base deprotection as follows.

### Base Deprotection for Solution Phase Synthesis:

The 3′-N-trityl protected PMO oligonucleotide residue from the final coupling step (1 wt.) was dissolved in MeOH (8 vols) and then 7N NH₃ in MeOH (20 vols) was added. The reaction mixture was heated to 50-55 °C for at least 48 hours. The solution was filtered to remove any solids, and rinsed with 1:1 MeOH/7N NH₃ in MeOH. Purification by preparative-scale chromatography using a reverse phase gradient as outlined in Table 15 afforded the 3′-N-trityl protected PMO after solvent evaporation.

TABLE 15
Analytical and purification conditions for stereopure
3′-N-trityl protected PMO oligonucleotides

| | |
|---|---|
| Analytical | |
| Column | XBridge, Premier BEH C18 300 Å 2.5 μm 150 × 2.1 mm |
| | (Part number: 186009894) |
| Flow rate | 0.8 mL/min |
| Column | room temperature |
| temperature | |
| Buffer A | 10 mM ammonium bicarbonate in water |
| Buffer B | 10 mM ammonium bicarbonate/MeOH/MeCN (10/10/80) |
| Gradient | 5 to 99% B (6 min) |
| Preparative | |
| Column | Phenomenex Luna C18 5 μm, 300 × 4.6 mm |
| | (Part number: 00H-4252-E0) |
| Flow rate | 1.5 mL/min |
| Column | 40 °C |
| temperature | |
| Buffer A | Water + 0.1% Et₃N |
| Buffer B | MeCN + 0.1% Et₃N |
| Gradient | 5 to 99% B (23 min) |

Final trityl deprotection: To the base-deprotected PMO oligonucleotide from HPLC purification was added 0.1N phosphoric acid (at least 20 equivalents) and the reaction was monitored by HPLC. Upon completion of trityl deprotection assessed by two consecutive HPLC runs, the reaction mixture was basified by addition of ammonium hydroxide (at least 40 equivalents). The solution was filtered and the final PMO oligonucleotide was purified by HPLC under the conditions in Table 16.

TABLE 16
Analytical and purification conditions for fully
deprotected stereopure PMO oligonucleotides

| | |
|---|---|
| Analytical | |
| Column | XBridge, Premier BEH C18 300 Å 2.5 μm 150 × 2.1 mm |
| | (Part number: 186009894) |
| Flow rate | 0.8 mL/min |
| Column | room temperature |
| temperature | |
| Buffer A | 10 mM ammonium bicarbonate in water |
| Buffer B | 10 mM ammonium bicarbonate/MeOH/MeCN (10/10/80) |
| Gradient | 5 to 99% B (6 min) |
| Preparative | |
| Column | Waters, XBridge Prep C18 OBD 5 μm, 19 × 100 mm |
| | (Part number: 186002978) |
| Flow rate | 25.5 mL/min |
| Column | room temperature |
| temperature | |
| Buffer A | 10 mM ammonium bicarbonate in water |
| Buffer B | 10 mM ammonium bicarbonate/MeOH/MeCN |
| | (10/10/80) |
| Gradient | 5 to 28% B (28 min) |

### Example 6: Analytical Data for Stereopure PMO Oligonucleotides

The Melting temperature (Tm) of PMO oligonucleotides:

Tm measurement device: Shimadzu UV-2700 UV-Vis Spectrophotometer

ASO samples were prepared by dissolving ~0.6-0.8 mg of solid to ~3.2 μg/mL using nuclease free water. Reverse complementary RNA (obtained from IDT Technologies Inc.) was dissolved to 400 μM in nuclease free water. 10 μL aliquots of each stock solution were diluted to 1 mL using nuclease free water to determine their concentrations by UV-Vis Spectrophotomer. Test Samples (500 μL) were prepared containing 4.0 μM PMO with 4.0 μM reverse complimentary RNA in buffer (100 mM NaCl, 10 mM Na Phosphate pH 7.0 with 0.1 mM EDTA). Test samples were incubated in a 1 mL cuvette and heated from 15 °C to 105 °C at 0.5 °C/min. UV absorbance increase due to strand melting was monitored at 260 nm. Prior to the experiment, the samples were melted and reannealed by heating from 25 °C to 95 °C at 5 °C/min and cooling to starting temperatures to ensure complete annealing. Shimadzu Tm Analysis software was used to calculate the Tm (curve inflection point: 50% melting) using the derivative function.

Analytical data for stereopure PMO oligonucleotides.

### PMO-424:

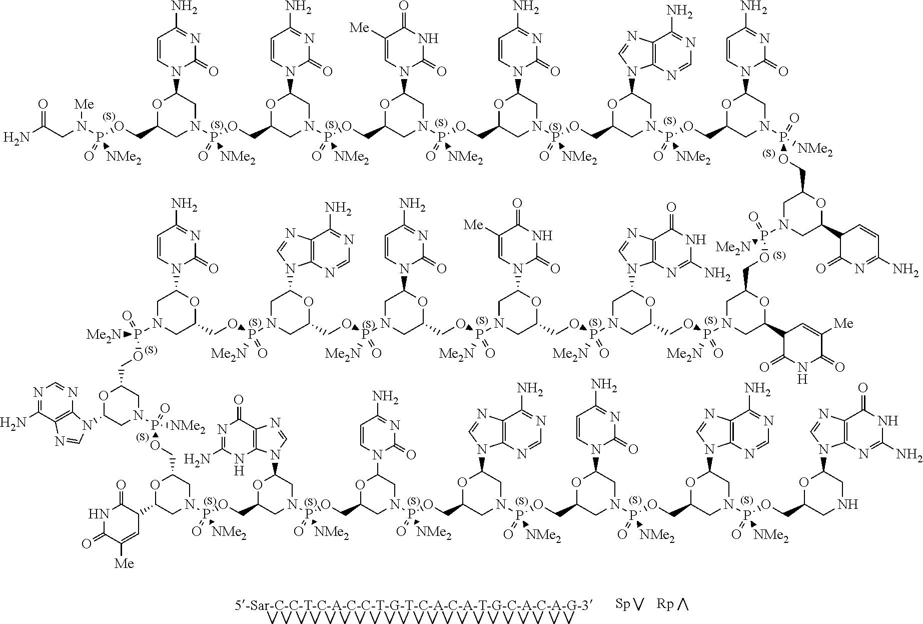

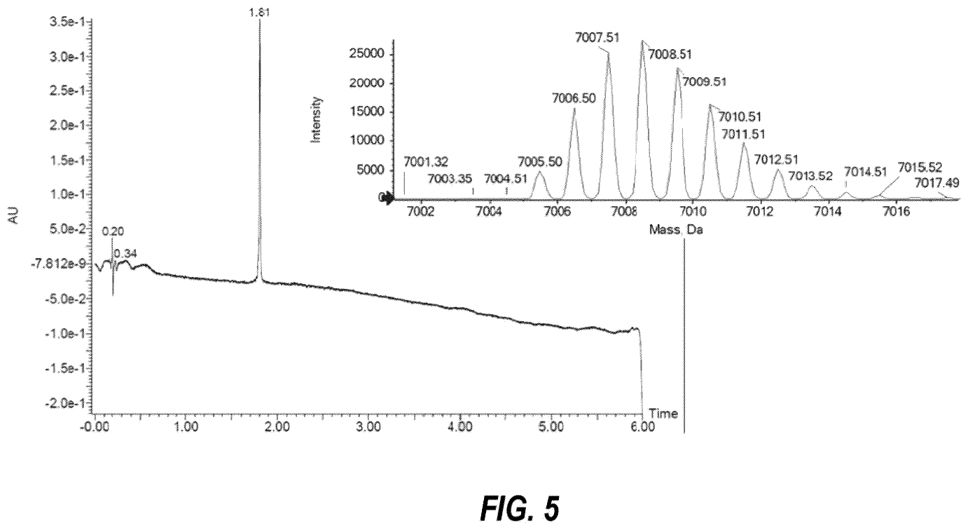

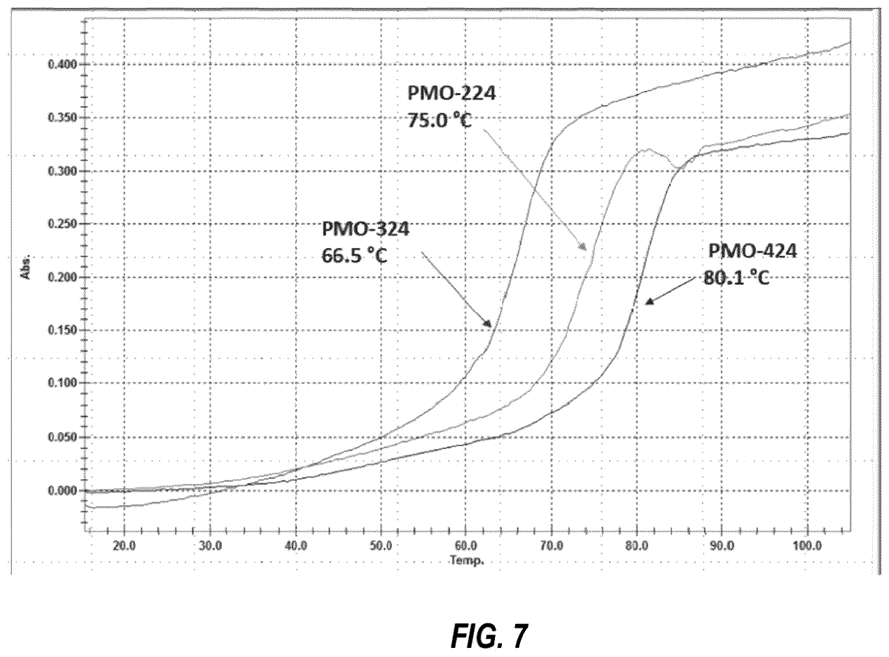

P³¹ NMR (D₂O, 162 MHz) δ 21.5, 18.7, 18.6, 18.5, 18.4, 18.3, 18.3, 18.1, 18.0, 17.9.
ESI-TOF-MS Calcd.: 7009.02 for C₂₄₆H₃₉₀N₁₁₉O₈₄P₂₁;
Found: 7008.51.
Tm=80.1 °C (Tm of stereorandom=75.0 OC). See FIG. 7.
See FIG. 5 for HPLC and HRMS data.

### PMO-324:

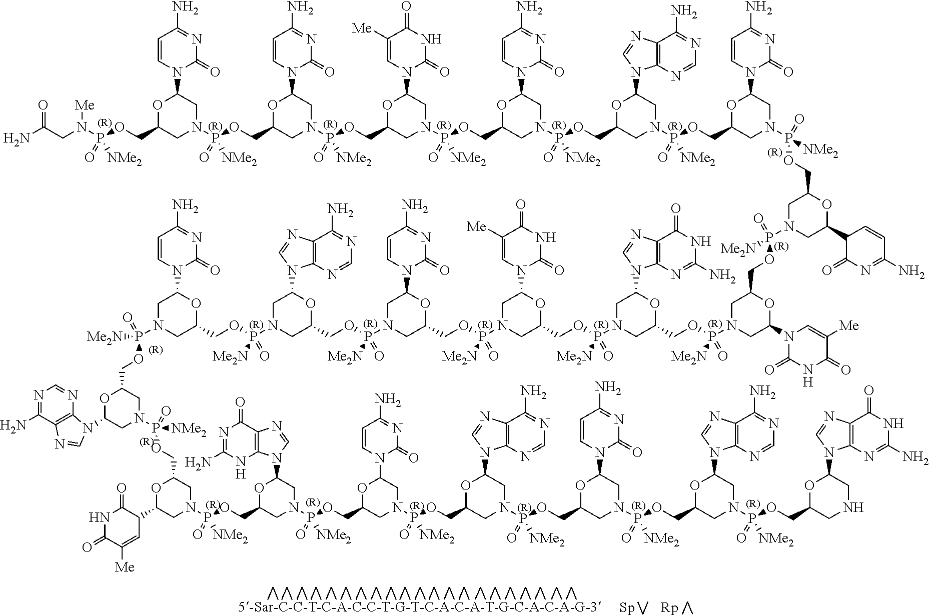

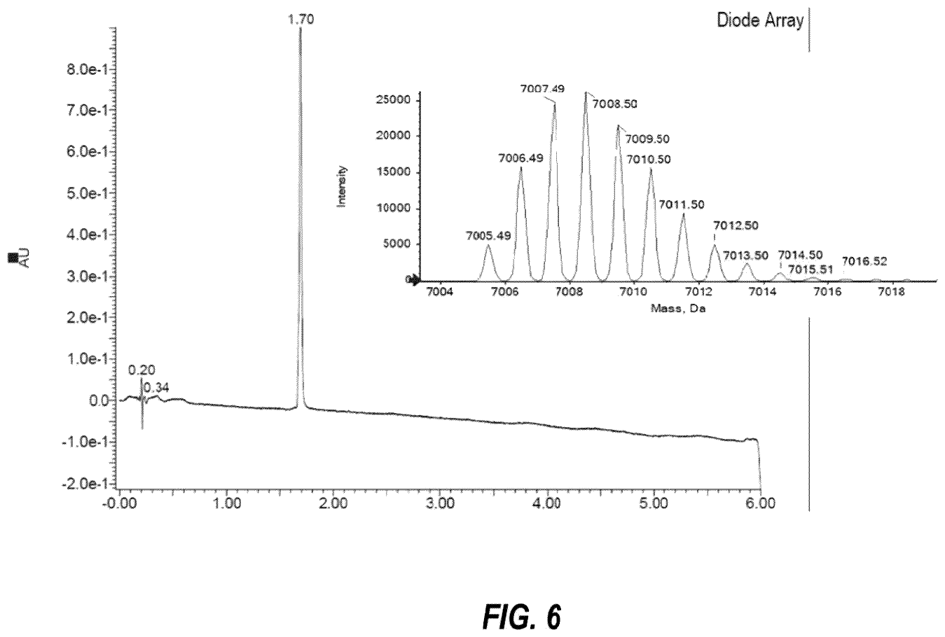

ESI-TOF-MS Calcd.: 7009.02 for C₂₄₆H₃₉₀N₁₁₉O₈₄P₂₁.
Found: 7008.50.
Tm=66.5 °C (Tm of stereorandom=75.0 °C). See FIG. 7.
See FIG. 6 for HPLC and HRMS data.

### PMO-502:

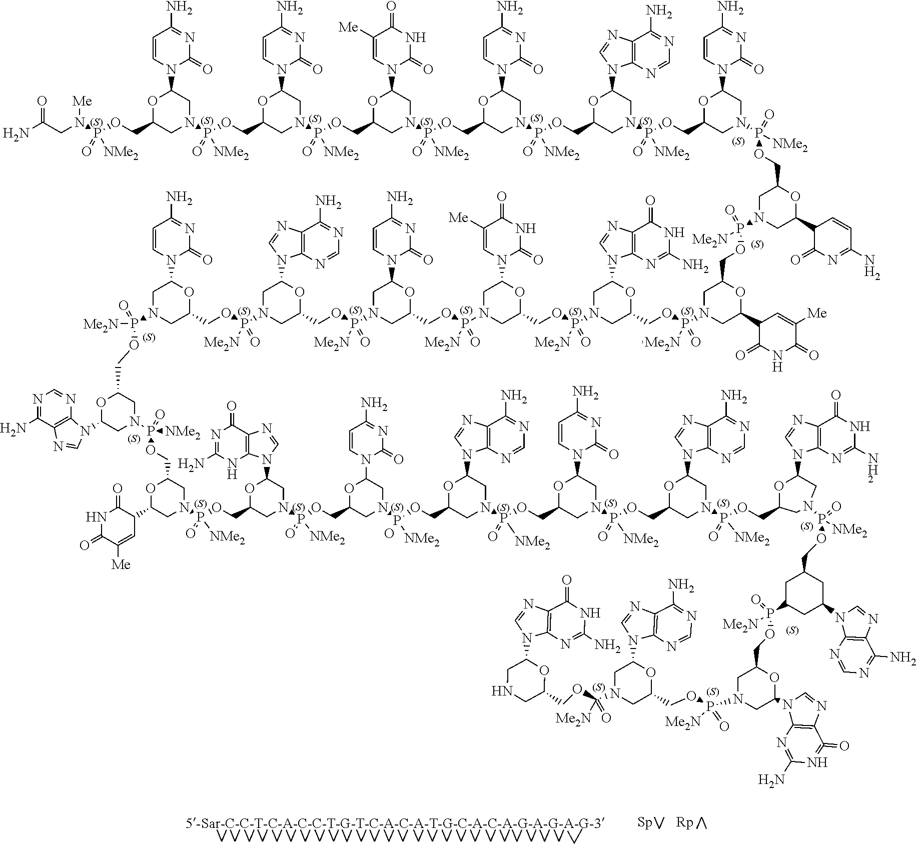

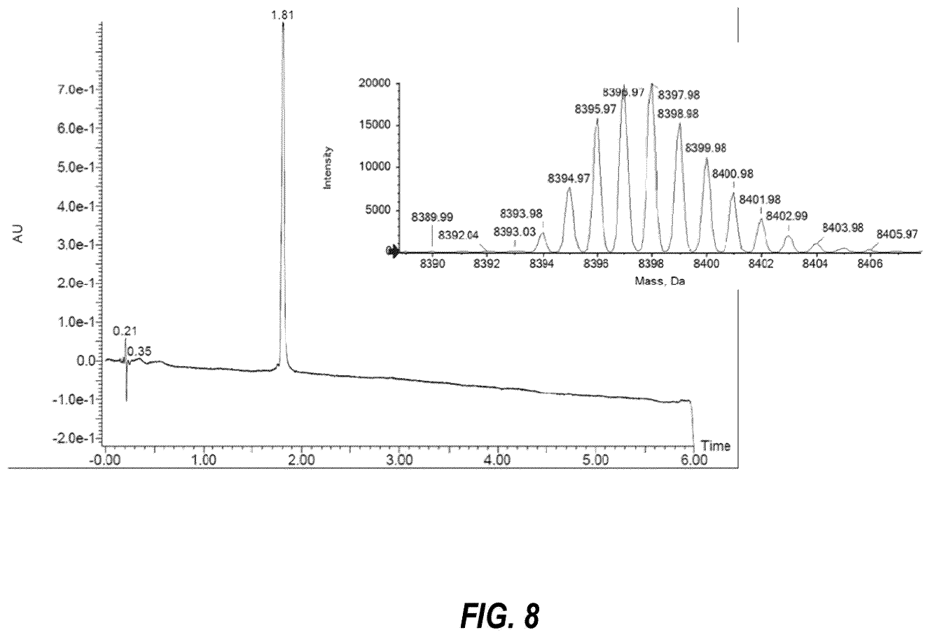

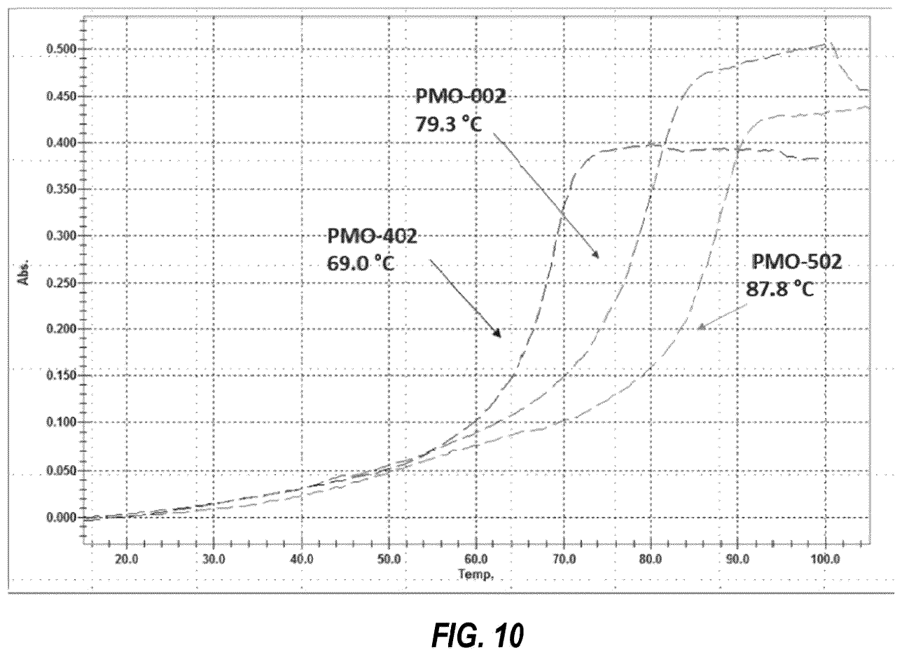

ESI-TOF-MS Calcd.: 8398.20 for C₂₉₄H₄₆₂N₁₄₇O₉₈P₂₅
Found: 8397.98.
Tm=87.8 °C (Tm of stereorandom=79.3 °C). See FIG. 10.
See FIG. 8 for HPLC and HRMS data.

### PMO-402:

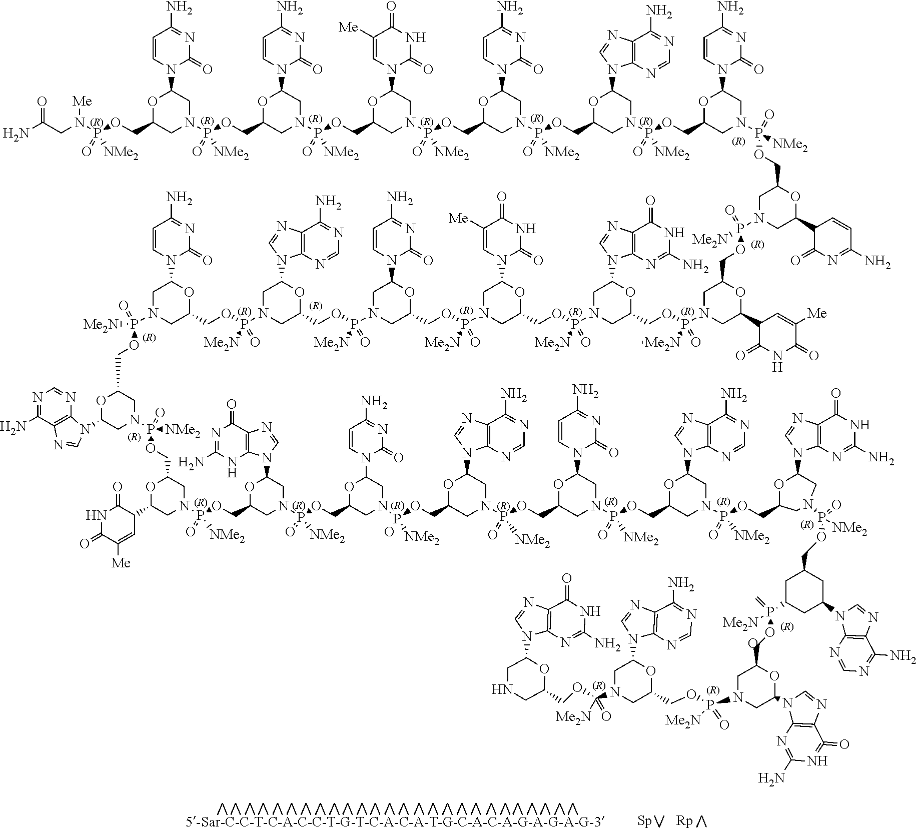

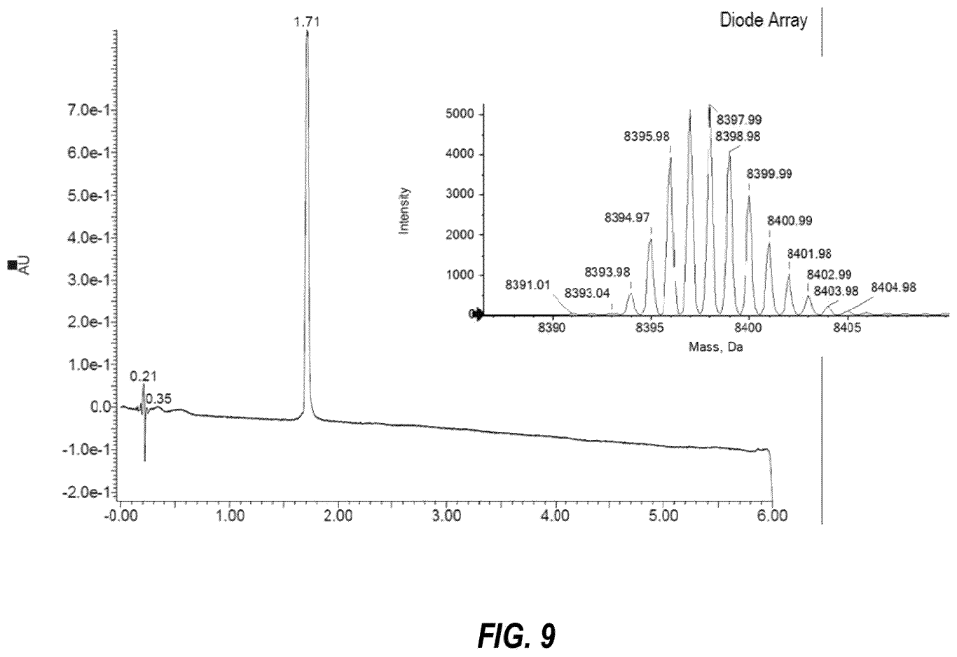

ESI-TOF-MS Calcd.: 8398.20 for C₂₉₄H₄₆₂N₁₄₇O₉₈P₂₅
Found: 8397.99.
Tm=69.0 °C (Tm of stereorandom=79.3 °C). See FIG. 10.
See FIG. 9 for HPLC and HRMS data.

### Example 7: Solid Phase Synthesis of Stereopure PMOs Using Peptide Synthesizer

### Deprotection of Fmoc on Sar-Wang Resin:

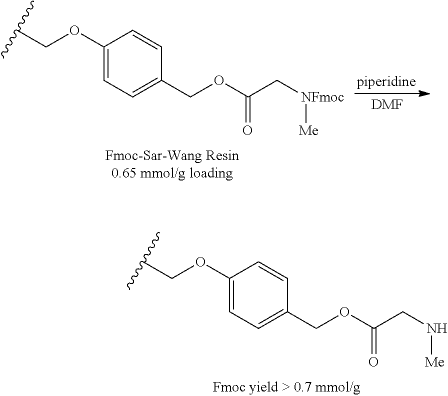

Fmoc-SAR-Wang resin (purchased from Aapptec, RWG103, Lot #9953380, 0.65 mmol/g, 110-200 mesh) (1 g, 650 mmol) was treated with DMF (8 mL), allowed resin to swell for 2 h and drained DMF. The resin was treated with 20% piperidine in DMF (6 mL), shaked for 3 minutes, removed solvent, and dried for 1 minute under N2 gas (repeated the same sequence for 4 times). Finally, the resin was washed with DMF (5 mL×5 times), washed with CH₂Cl₂ (5 mL×5 times), and dried under vacuum using N2 gas for overnight to give 0.8 g of resin.

Calculation of resin loading: To the collected piperidine solution was added 20% piperidine in DMF to make final volume of 40 mL. Now, 0.1 mL of solution was diluted 100 times with DMF and measured UV absorbance at 301 nm of the Fmoc group per gram. The loading amount of the resin was >700 μmol/g.
Conditions for UV measurement
Solvent: 20% piperidine in DMF
Wave length: 301 nm
ε=7800

### General Procedure for Solid-Phase Synthesis of PMOs:

(a) Synthetic Flow for PMOs with all-Sp Internucleotide Linkages

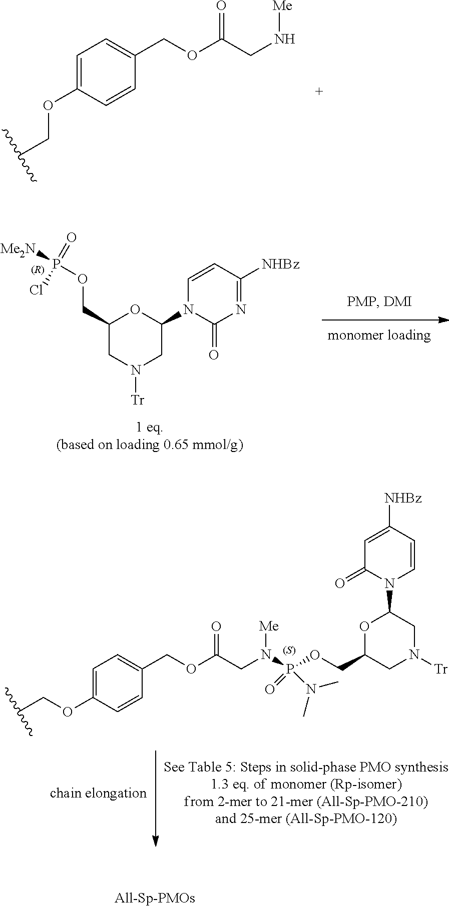

(b) Synthetic Flow for PMOs with all-Rp Internucleotide Linkages

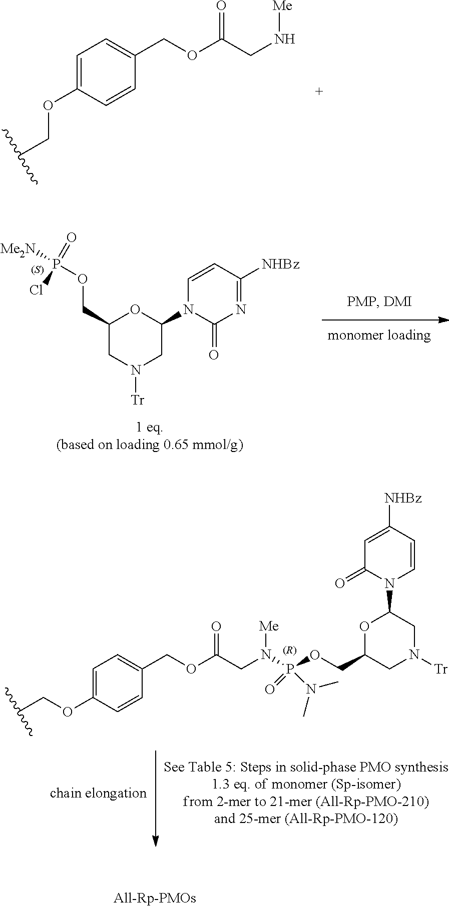

Fmoc deprotected resin (1.10 g, loading: 0.650 mmol/g) was transferred into the peptide synthesizer reaction vessel, washed with CH₂Cl₂ (20 mL×5 times), washed with acetonitrile (20 mL×5 times), and dried. Stereopure cytosine dimethylphosphoramidochloridate (1 eq.) was added to flask as a solid. Then, 1,2,2,6,6-pentamethylpiperidine (PMP, 10.0 eq.) and anhydrous 1,3-dimethyl-2-imidazolidinone (DMI, 5.0 mL) were added to vessel and shaked at room temperature for 20 hours. LCMS of the reaction aliquot showed no monomer in the solution (indicates all monomer was loaded on resin). Then, steps 5-9 in Table 17 were performed.

TABLE 17
Steps in solid-phase PMO synthesis

| Step | Reaction | Reagent | | |
|---|---|---|---|---|
| 1 | Detritylation | 2% 3-cyanopyridine-TFA, 0.9% EtOH, 20% | 7 | cycles |
| | | TFE/DCM (80 mL) then CH₂Cl₂ (15 mL × 2) | | |
| 2 | Neutralization | 10% Hunig's base in NMP | 30 ml × | |
| | | | 4 times | |
| 3 | Wash | 1,3-dimethyl-2-imidazolidinone (30 mL × 2 times) | | |
| | | MeCN (25 mL × 10 times), | | |
| | | CH₂Cl₂ (25 mL × 15 times) | | |
| 4 | Coupling | Monomer (1.3 eq), DMI (0.05M), PMP (10 eq.) | 20 | h |
| 5 | Wash | MeCN (25 mL × 10 times), | | |
| | | CH₂Cl₂ (25 mL × 10 times) | | |
| 6 | Capping | 2,6-Lutidine (30% in MeCN, 6 mL) Ac₂O (20% in | 3 | times |

| | | | |
|---|---|---|---|
| | | MeCN, 6 mL) | |
| 7 | Wash | CH₂Cl₂ (25 mL × 10 times) | |
| 8 | Ac₂O removal | 10% Morpholine in NMP | 40 ml × |
| | | | 4 times |
| 9 | Wash | CH₂Cl₂ (30 ml × 10 times) | |

### Preparation of Detritylation Solution:

To a solution of 4-cyanopyridine (10.1 g; 1.055 eq) in dichloromethane (790 mL) is added trifluoroacetic acid (10.5 g; 1.0 eq), followed by 2,2,2-trifluoroethanol (198 mL) and ethanol (10 mL), and the solution is stirred for 3 hours.

After the first monomer loading on resin, the synthetic cycle (as shown in Table 17) was started. The synthesis had a series of iterative steps including deprotection/neutralization/coupling/capping. The required monomer (purities of monomers were characterized by HPLC-Mass before use) was added in each cycle to obtain the titled nucleotide sequence.

In each synthetic cycle, after coupling reaction (step 4, Table 17) a bit of resin was subjected to cleavage conditions (0.1 mL of 7N NH₃/MeOH, at 55 °C, 4 h), and recorded RP HPLC-Mass for coupling efficiency (RP HPLC-Mass showed two peaks for methyl ester and amide in ~2:1 ratio. For complete conversion of methyl ester to amide, the cleavage reaction was left overnight stirring at 55 °C). The cleavage protocol was iterated from 2-mer to 21-mer for PMO-324 and PMO-424 and to 25-mer for PMO-402 and PMO-502. The RP HPLC-Mass was recorded using conditions in Table 18.

TABLE 18
Analytical conditions for reaction monitoring in PMO synthesis.

Analytical

| | |
|---|---|
| Column | Acquity UPLC BEH C18 1.7 μm 50 × 2.1 mm |
| | (Part number: 186002350) |
| Flow rate | 0.8 mL/min |
| Column | 60 °C |
| temperature | |
| Buffer A | 10 mM ammonium bicarbonate in water |
| Buffer B | 10 mM ammonium bicarbonate/MeOH/MeCN (10/10/80) |
| Gradient | 5 to 99% B (6 min) |

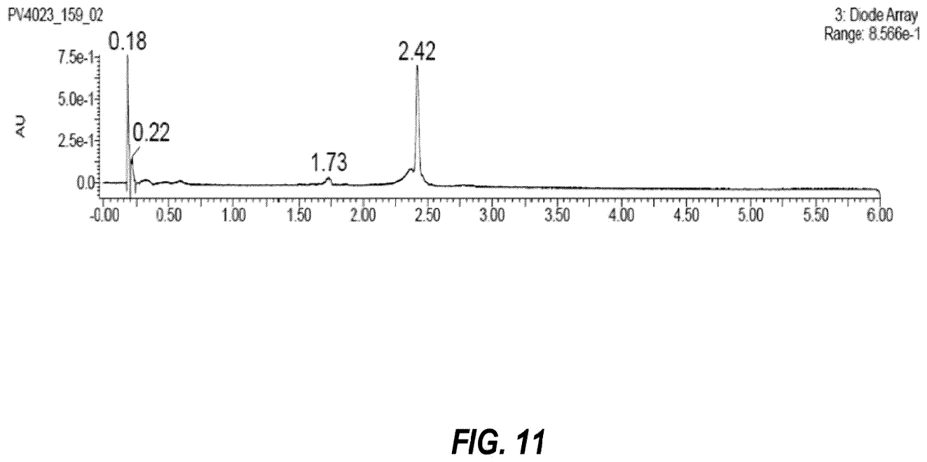

For example, FIG. 11 shows the UV chromatogram of trityl-protected 21-mer (all-Sp-Sar-CCTCACCTGTCACATGCACAG-Tr) after cleavage from resin.

Cleavage from the Resin and Base Deprotection:

After completion of desired oligonucleotide length, the synthesized PMO-loaded resin was dried, transferred to centrifugal bottle, and charged with 7N NH₃/MeOH (~0.5 mL/1 μmol). The mixture was stirred at 50-55 °C for 60 hours. The reaction was cooled to room temperature, filtered the solids, and washed with methanol. The resulting filtrate was concentrated under reduced pressure to approximate final volume of ~20 mL, then, filtered any solids over 0.4 micron membrane filter. The filtrate was concentrated to dryness and weighed. The obtained crude residue was dissolved with 60 mL of solvent mixture of aq. 50 mM Et₃NHOAc (used cell culture water)/MeCN (1/1) with Et₃N (0.1%). The filtrate was purified by reversed phase HPLC conditions as shown in Table 19.

TABLE 19
Analytical and purification conditions for
sterepure 3′-N-trityl protected PMOs.

| | |
|---|---|
| Analytical | |
| Column | XBridge, Premier BEH C18 300 Å 2.5 μm 150 × 2.1 mm |
| | (Part number: 186009894) |
| Flow rate | 0.8 mL/min |
| Column | 60 °C |
| temperature | |
| Solution A | 10 mM ammonium bicarbonate in water |
| Solution B | 10 mM ammonium bicarbonate/MeOH/MeCN |
| | (10/10/80) |
| Gradient | 5 to 99% B (6 min) |
| Preparative | |
| Column | Waters, XBridge Prep C18 OBD 5 μm, 19 × 100 mm |
| | (Part number: 186002978) |
| Flow rate | 25.5 mL/min |
| Column | room temperature |
| temperature | |
| Solution A | 10 mM ammonium bicarbonate in water |
| Solution B | 10 mM ammonium bicarbonate/MeOH/MeCN (10/10/80) |
| Gradient | 5 to 38% B (28 min) |

### Final Detritylation:

To a flask containing the recovered 3′-N-Tr-PMO (1 eq.) was added freshly prepared 0.1 M aq. phosphoric acid (20 eq.) and the mixture was stirred at room temperature for 2 hours (a white turbid solution was formed within 10 minutes). Reaction completion was checked by two consecutive LCMS runs (shows staring material peak was converted to an earlier eluting peak. HPLC sample was prepared in water only). The reaction was basified by the addition of 28% ammonium hydroxide (40 eq.), stirred for 30 min, filtered the solids through membrane filter (0.45 μm), and washed with water. The resulting filtrate was purified by reverse phase HPLC (Table 20).

TABLE 20
Analytical and purification conditions
for fully deprotected stereopure PMOs

| | |
|---|---|
| Analytical | |
| Column | XBridge, Premier BEH C18 300 Å 2.5 μm 150 × 2.1 mm |
| | (Part number: 186009894) |
| Flow rate | 0.8 mL/min |
| Column | 60 °C |
| temperature | |
| Solution A | 10 mM ammonium bicarbonate in water |
| Solution B | 10 mM ammonium bicarbonate/MeOH/MeCN (10/10/80) |
| Gradient | 5 to 99% B (6 min) |
| Preparative | |
| Column | Waters, XBridge Prep C18 OBD 5 μm, 19 × 100 mm |
| | (Part number: 186002978) |
| Flow rate | 25.5 mL/min |
| Column | room temperature |
| temperature | |
| Solution A | 10 mM ammonium bicarbonate in water |
| Solution B | 10 mM ammonium bicarbonate/MeOH/MeCN |
| | (10/10/80) |
| Gradient | 5 to 28% B (28 min) |

Each fraction was analyzed (on HPLC) and the product containing fractions were dried using Genevac. The final product was dissolved in endotoxin-free water, the solution was filtered through Amicon 3K filter to remove any inorganic salt impurities. The aqueous solution obtained was freeze-dried to give the title compound as a white cotton-like solid.

### Analytical Data for Stereopure PMOs Prepared by Solid-Phase Synthesis:

### PMO-424:

P³¹ NMR (D₂O, 162 MHz) δ 21.5, 18.7, 18.6, 18.5, 18.4, 18.3, 18.3, 18.1, 18.0, 17.9.

LRMS: Calcd. m/z for [M+5H]⁵⁺ ion of C₂₄₆H₃₉₀N₁₁₉O₈₄P₂₁ (m/z=7009.02): 1402.80. found: 1402.62.

### PMO-324:

LRMS: Calcd. m/z for [M+5H]⁵⁺ ion of C₂₄₆H₃₉₀N₁₁₉O₈₄P₂₁ (m/z=7009.02): 1402.80. found: 1403.4.

### PMO-402:

LRMS: Calcd. m/z for [M+6H]⁶⁺ ion of C₂₉₄H₄₆₂N₁₄₇O₉₈P₂₅ (m/z=8396.92): 1400.66. found: 1401.2.

### Example 8: Additional Exemplary MOE-ASOs

Phosphorothioate oligonucleotides were designed for screening. All oligonucleotides listed in Table 21 below contain ribonucleotides with phosphorothioate backbone except when noted (e.g. solid line (−)=phosphodiester (PO) bond). All oligonucleotides listed in Table 21 below were synthesized with 5-methylcytosine ribonucleotide. All oligonucleotides listed in Table 21 below have stereorandom phosphorothioate internucleotide linkages, and thus are called stereorandom oligonucleotides. All oligonucleotides listed in Table 21 are complementary to Region 6: (SEQ ID NO:218).

TABLE 21
ASOs targeting CD33

| | | | MW (free | |
|---|---|---|---|---|
| | | SEQ | form | MS |
| ASO# | Sequence (5′ to 3′) | ID No: | calculated) | observed |
| MOE-245 | CTCCATCCGAAAGAAGTATG | 245 | 7966.9 | 7967.1 |
| MOE-246 | TCCATCCGAAAGAAGTATGA | 246 | 7976.9 | 7976.9 |
| MOE-247 | CCATCCGAAAGAAGTATGAA | 247 | 7985.9 | 7986.9 |
| MOE-248 | CATCCGAAAGAAGTATGAAC | 248 | 7985.9 | 7986.4 |
| MOE-249 | TCCGAAAGAAGTATGAACCA | 249 | 7985.9 | 7986.6 |
| MOE-250 | CCGAAAGAAGTATGAACCAT | 250 | 7985.9 | 7986.2 |
| MOE-251 | ATCCGAAAGAAGTATGAA | 251 | 7199.2 | 7199.9 |
| MOE-252 | CCGAAAGAAGTATGAACC | 252 | 7188.1 | 7188.3 |
| MOE-253 | TCCGAAAGAAGTATGAAC | 253 | 7189.2 | 7188.2 |
| MOE-254 | CCATCCGAAAGAAGTATG | 254 | 7179.2 | 7179.1 |
| MOE-255 | TCCATCCGAAAGAAGTAT | 255 | 7154.2 | 7154.2 |
| MOE-256 | GAAAGAAGTATGAACCAT | 256 | 7199.2 | 7197.9 |
| MOE-257 | ATC-CGAAAGAAGTATGA-ACC | 12 | 7953.8 | 7953.9 |
| MOE-258 | ATCC-GAAAGAAGTATG-AACC | 12 | 7953.8 | 7953.3 |
| MOE-259 | ATCCG-AAAGAAGTAT-GAACC | 12 | 7953.8 | 7953.4 |
| MOE-260 | ATCCG-AAAGAAGTA-TGAACC | 12 | 7953.8 | 7953.4 |
| MOE-261 | ATCC-GAAAGA-AGTATG-AACC | 12 | 7937.7 | 7937.3 |
| MOE-262 | ATCC-gAAAGAAGTATGAACC | 12 | 7923.8 | 7924.3 |
| MOE-263 | ATCC-gAAAGAAGTATG-aACC | 12 | 7861.7 | 7861.9 |
| MOE-264 | ATCC-gAAAGAaGTATG-aACC | 12 | 7815.6 | 7815.8 |
| MOE-265 | CCGA-aAGAAGTATGAACC | 252 | 7126.1 | 7126.5 |
| MOE-266 | CCGA-aAGAAGTATG-aACC | 252 | 7064.0 | 7064.1 |
| MOE-267 | CCGA-aAGAAGtATG-aACC | 252 | 7017.9 | 7018.0 |
| MOE-268 | CCG-AAAGAAGTATGA-ACC | 252 | 7156.1 | 7156.7 |
| MOE-269 | CCGA-AAGAAGTATG-AACC | 252 | 7156.1 | 7156.6 |
| MOE-270 | CCGAA-AGAA-GTATG-AACC | 252 | 7140.0 | 7140.7 |
| MOE-271 | CCGAA-AGAAGTAT-GAACC | 252 | 7156.1 | 7156.7 |
| MOE-272 | CCG-A-AAGAAGTATGAACC | 252 | 7156.1 | 7156.7 |
| MOE-273 | CCG-AA-AGAAGTATGAACC | 252 | 7156.1 | 7156.8 |
| MOE-274 | CCGAAAGAAGTATG-A-ACC | 252 | 7156.1 | 7156.8 |
| MOE-275 | mAm TfCfCfGfAfAfAfGfAfAfGfTfAfTfGfAf | 12 | 6814.1 | 6814.7 |
| | AmCmC | | | |
| MOE-276 | fAfTfCfCfGmAmAmAmGmAmAmGmTm | 12 | 6862.3 | 6862.9 |
| | AfTfGfAfAfCfC | | | |

X (A, T, C, G) = 2′-MOE ribonucleotide, C = 5-Methyl cytosine, lower case letter = LNA (locked nucleic acid), (-) = PO bond, fX = 2′-fluoro ribonucleotide, mX = 2′-OMe ribonucleotide

All oligonucleotides listed in Table 22 below contain a 2′-O-MOE modified ribonucleotides and a hydroxyl group at the 5′ end. Oligonucleotides in Table 22 contain stereopure phosphorothioate internucleotide linkages, and thus are called stereopure MOE oligonucleotides. All oligonucleotides listed in Table 22 are complementary to Region 6: (SEQ ID NO:218).

TABLE 22
Stereopure ASOs targeting CD33

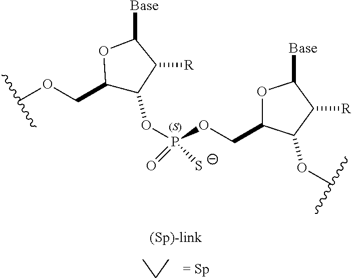

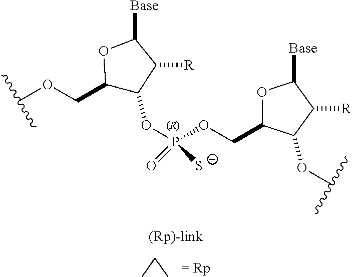

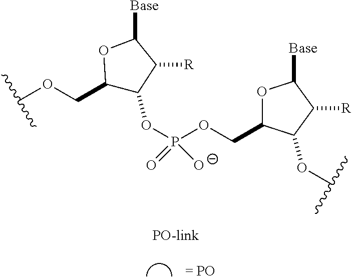

| | | SEQ |
|---|---|---|
| ASO# | Sequence (5' to 3') | ID No: |
| MOE- 277 | 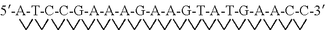 | 12 |

| | | |
|---|---|---|
| MOE- 278 | 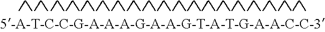 | 12 |

| | | |
|---|---|---|
| MOE- 279 | 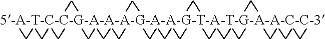 | 12 |

| | | |
|---|---|---|
| MOE- 280 | 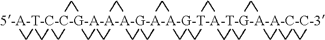 | 12 |

| | | |
|---|---|---|
| MOE- 281 | 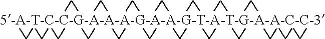 | 12 |

| | | |
|---|---|---|
| MOE- 282 | 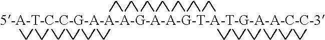 | 12 |

| | | |
|---|---|---|
| MOE- 283 | 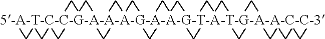 | 12 |

| | | |
|---|---|---|
| MOE- 284 | 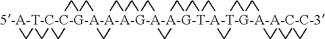 | 12 |

| | | |
|---|---|---|
| MOE- 285 | 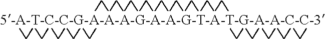 | 12 |

| | | |
|---|---|---|
| MOE- 286 | 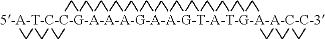 | 12 |

| | | |
|---|---|---|
| MOE- 287 | 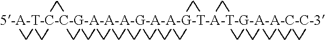 | 12 |
| MOE- 288 | 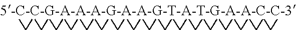 | 252 |
| MOE- 289 | 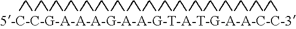 | 252 |
| MOE- 290 | 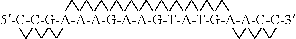 | 252 |
| MOE- 291 | 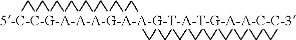 | 252 |
| MOE- 292 | 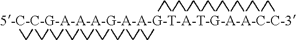 | 252 |
| MOE- 293 | 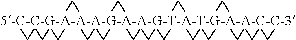 | 252 |
| MOE- 294 | 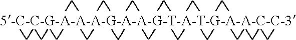 | 252 |
| MOE- 295 | 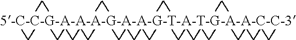 | 252 |
| MOE- 296 | 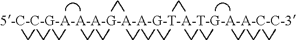 | 252 |
| MOE- 297 | 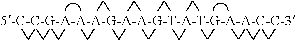 | 252 |
| MOE- 298 | 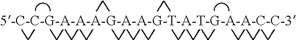 | 252 |
| MOE- 299 | 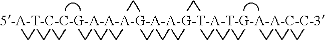 | 12 |
| MOE- 300 | 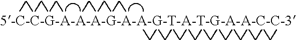 | 252 |
| MOE- 301 | 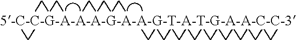 | 252 |
| MOE- 303 | 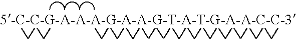 | 252 |
| MOE- 304 | 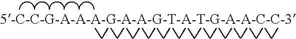 | 252 |
| MOE- 305 | 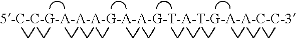 | 252 |
| MOE- 306 | 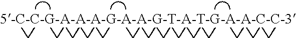 | 252 |
| MOE- 307 | 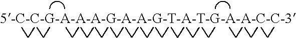 | 252 |
| MOE- 308 | 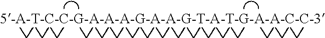 | 12 |

| | | |
|---|---|---|
| MOE- 309 | 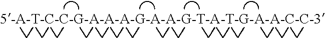 | 12 |
| MOE- 310 | 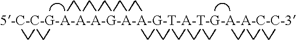 | 252 |
| MOE- 311 | 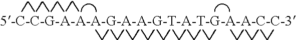 | 252 |

### Example 9: Preparation of Stereopure 2′-MOE Phosphorothiolate Oligonucleotides

### Protected 2′-O-MOE-3′-OH Monomers

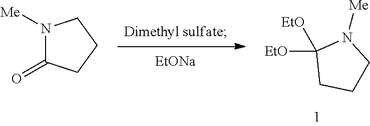 [SMILES: "EtONa" ; CN1CCCC1=O ; CCOC1(OCC)CCCN1C]

(Compound 1) 2,2-diethoxy-1-methylpyrrolidine: A mixture of NMP (100 mL, 1039.008 mmol) and dimethyl sulfate (99 mL, 1039.008 mmol) was stirred and heated to 80 °C (sand bath) overnight, then allowed to cool to rt. After cooling, the homogeneous liquid was washed with ether (2×100 mL) and the residual solvent was removed in vacuo. The obtained residue was dissolved in CH₂Cl₂ (400 mL), dried over anhydrous MgSO₄, filtered, washed with CH₂Cl₂ (100 mL) and concentrated under reduced pressure to give 5-methoxy-1-methyl-3,4-dihydro-2H-pyrrol-1-ium as a brown color viscous liquid (solidified at −20 °C storage); ¹H NMR (400 MHz, CDCl₃) δ 4.35-4.40 (m, 3H), 3.98-4.05 (m, 2H), 3.69-3.73 (m, 3H), 3.31-3.38 (m, 2H), 3.19-3.22 (m, 3H), 2.37-2.48 (m, 2H).

The crude product (obtained above) was added to a solution of sodium ethanolate (370 g, 1142.909 mmol, 21% sodium ethoxide in ethanol) at 50 to 55 °C over 1 hour by cannula or dropping funnel under N₂ atmosphere. After stirring at the same temperature for 3 hours, the reaction was cooled to room temperature. The precipitated white solid was filtered, washed with ethanol (50 mL) and the filtrate was concentrated (maintain water-bath temperature ~30 °C). Fractional distillation of crude residue under house vacuum at 55-65 °C gave 2,2-diethoxy-1-methylpyrrolidine (115 g, 66% yield) as a pale yellow or colorless liquid. The pure product was stored at −20 °C; ¹H NMR (400 MHz, CDCl₃) δ 3.44-3.60 (m, 4H), 2.83-2.91 (m, 3H), 2.33-2.40 (m, 4H), 1.90-1.98 (m, 2H), 1.72-1.87 (m, 2H), 1.15-1.22 (m, 6H).

### General Procedure 1: Pya (N-Methylpyrrolidine) Protection of 2′-O-MOE G, A, and mC

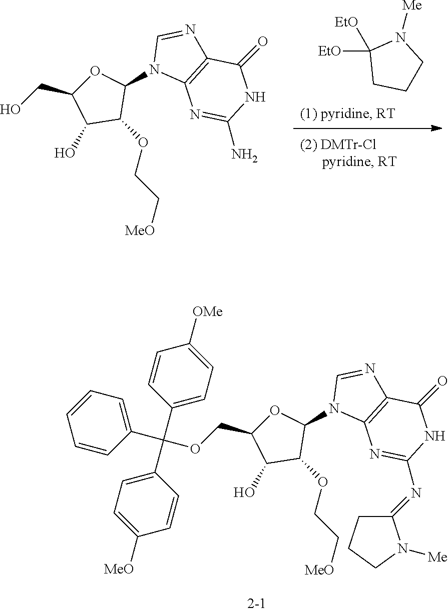 [SMILES: COCCO[C@@H]1[C@H](O)[C@@H](CO)O[C@H]1n1cnc2c(=O)[nH]c(N)nc21 ; CCOC1(OCC)CCCN1C ; COCCO[C@@H]1[C@H](O)[C@@H](COC(c2ccccc2)(c2ccc(OC)cc2)c2ccc(OC)cc2)O[C@H]1n1cnc2c(=O)[nH]c(/N=C3\CCCN3C)nc21]

### (Compound 2-1) 9-((2R,3R,4R,5R)-5-((bis(4-methoxyphenyl)(phenyl)methoxy)methyl)-4-hydroxy-3-(2-methoxyethoxy)tetrahydrofuran-2-yl)-2-(1-methylpyrrolidin-2-ylidene)amino)-1,9-dihydro-6H-purin-6-one

2-amino-9-((2R,3R,4R,5R)-4-hydroxy-5-(hydroxymethyl)-3-(2-methoxyethoxy)tetrahydrofuran-2-yl)-1,9-dihydro-6H-purin-6-one (13.8 g, 40.431 mmol) was chased under vacuum with anhydrous pyridine (100 mL) for two times. To the concentrated residue was added anhydrous pyridine (114 mL, 1418.073 mmol) followed by 2,2-diethoxy-1-methylpyrrolidine (14.01 g, 80.862 mmol) slowly at room temperature. The reaction was stirred at room temperature overnight, changing from a white turbid solution to a brown clear solution. Water (0.1 mL/6 mmol) was added, and the mixture was concentrated under vacuum, then chased with pyridine and MeCN 3 times. To the resulting residue were added pyridine (105 mL, 1298.197 mmol) and 1-[chloro-(4-methoxyphenyl)-phenylmethyl]-4-methoxybenzene (15.62 g, 46.108 mmol) at room temperature. After stirring at rt overnight, the reaction mixture was worked up with saturated NaHCO₃ (150 mL) and EtOAc (300 mL×2), and the residue was purified by a silica-gel column chromatography (100 g Star Silica, EtOAc/Hept 30 to 100% then EtOAc/MeOH 0 to 30%) to give Compound 2-1 as a foamy solid in 77% yield; ¹H NMR (400 MHz, CDCl3) δ 9.28-9.36 (m, 1H), 7.67-7.72 (m, 1H), 7.32-7.39 (m, 2H), 7.16-7.28 (m, 6H), 7.08-7.16 (m, 1H), 6.69-6.78 (m, 4H), 5.92-5.96 (m, 1H), 4.29-4.37 (m, 2H), 4.11-4.17 (m, 1H), 3.74-3.82 (m, 1H), 3.68-3.73 (m, 7H), 3.55-3.63 (m, 1H), 3.45-3.52 (m, 1H), 3.34-3.41 (m, 3H), 3.25-3.33 (m, 5H), 3.01-3.09 (m, 2H), 2.92-2.96 (m, 3H), 1.90-2.00 (m, 2H); MS (ESI, m/z) calculated for [C₃₉H₄₄N₅O₈+H⁺] 725.33. found 725.4.

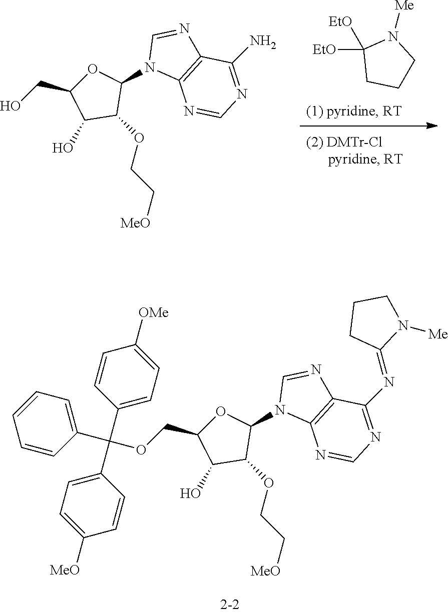 [SMILES: COCCO[C@@H]1[C@H](O)[C@@H](CO)O[C@H]1n1cnc2c(N)ncnc21 ; CCOC1(OCC)CCCN1C ; COCCO[C@@H]1[C@H](O)[C@@H](COC(c2ccccc2)(c2ccc(OC)cc2)c2ccc(OC)cc2)O[C@H]1n1cnc2c(/N=C3\CCCN3C)ncnc21]

### (Compound 2-2) (2R,3R,4R,5R)-2-((bis(4-methoxyphenyl)(phenyl)methoxy)methyl)-4-(2-methoxyethoxy)-5-(6-1-methylpyrrolidin-2-ylidene)amino)-9H-purin-9-yl)tetrahydrofuran-3-ol

Prepared according to general procedure 1, foamy solid, 89% yield; ¹H NMR (400 MHz, DMSO-d₆) δ 8.36 (d, J=8.0 Hz, 2H), 7.40-7.31 (m, 2H), 7.29-7.16 (m, 7H), 6.87-6.77 (m, 4H), 6.07 (d, J=4.8 Hz, 1H), 5.18 (d, J=6.0 Hz, 1H), 4.69 (t, J=5.2 Hz, 1H), 4.44 (q, J=5.2 Hz, 1H), 4.11-4.05 (m, 1H), 3.76-3.71 (m, 7H), 3.62 (dt, J=11.2, 4.8 Hz, 1H), 3.49 (t, J=7.2 Hz, 2H), 3.42 (t, J=4.8 Hz, 2H), 3.23 (d, J=4.8 Hz, 2H), 3.14 (s, 3H), 3.04 (s, 3H), 2.85 (t, J=8.0 Hz, 2H), 2.02-1.93 (m, 2H); MS (ESI, m/z) calculated for [C₃₉H₄₄N₆O₇+H⁺] 709.33. found 709.20.

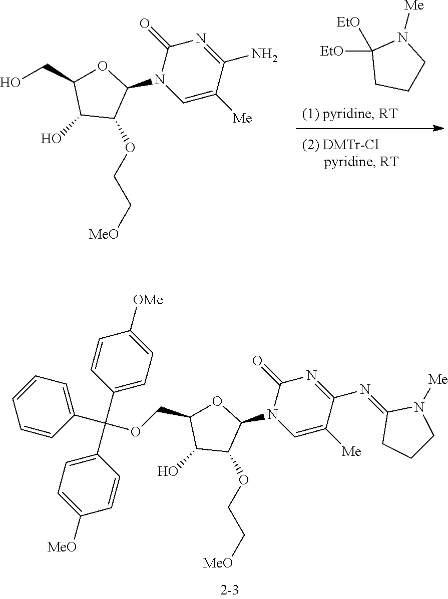 [SMILES: COCCO[C@@H]1[C@H](O)[C@@H](CO)O[C@H]1n1cc(C)c(N)nc1=O ; CCOC1(OCC)CCCN1C ; COCCO[C@@H]1[C@H](O)[C@@H](COC(c2ccccc2)(c2ccc(OC)cc2)c2ccc(OC)cc2)O[C@H]1n1cc(C)c(/N=C2\CCCN2C)nc1=O]

### (Compound 2-3) 1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-4-(1-methylpyrrolidin-2-ylidene)amino)pyrimidin-2(1H)-one

Prepared according to general procedure 1, 87% yield; foamy solid; 1H NMR (400 MHz, CDCl₃) δ 7.77-7.81 (m, 1H), 7.46-7.51 (m, 2H), 7.34-7.41 (m, 4H), 7.27-7.33 (m, 2H), 7.20-7.27 (m, 1H), 6.82-6.88 (m, 4H), 5.99-6.04 (m, 1H), 4.34-4.43 (m, 1H), 4.25-4.33 (m, 1H), 4.08-4.15 (m, 1H), 3.99-4.05 (m, 1H), 3.90-3.99 (m, 1H), 3.74-3.83 (m, 6H), 3.54-3.64 (m, 3H), 3.42-3.50 (m, 3H), 3.42 (s, 3H), 3.29-3.34 (m, 1H), 3.07-3.29 (m, 2H), 3.03-3.07 (m, 3H), 2.00-2.11 (m, 2H), 1.53-1.58 (m, 3H); MS (ESI, m/z) calculated for [C₃₉H₄₄N₆O₈+H⁺] 699.33. found 699.25.

### Pivaloylmethyl (POM) Protection of T:

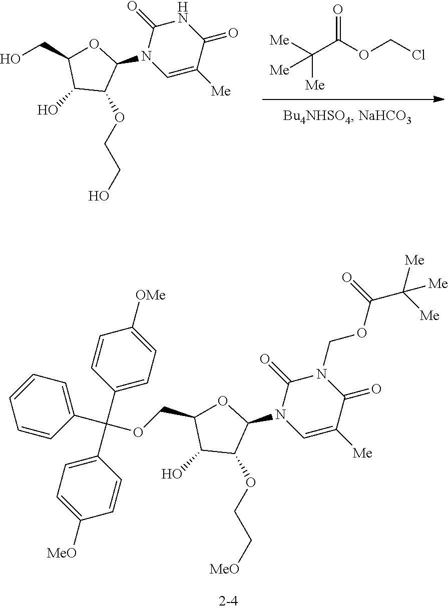 [SMILES: Cc1cn([C@@H]2O[C@H](CO)[C@@H](O)[C@H]2OCCO)c(=O)[nH]c1=O ; CC(C)(C)C(=O)OCCl ; COCCO[C@@H]1[C@H](O)[C@@H](COC(c2ccccc2)(c2ccc(OC)cc2)c2ccc(OC)cc2)O[C@H]1n1cc(C)c(=O)n(COC(=O)C(C)(C)C)c1=O]

### (Compound 2-4) (3-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-2,6-dioxo-3,6-dihydropyrimidin-1(2H)-yl)methyl Pivalate

Step 1: To 1-((2R,3R,4R,5R)-4-hydroxy-5-(hydroxymethyl)-3-(2-methoxyethoxy)tetrahydrofuran-2-yl)-5-methylpyrimidine-2,4(1H,3H)-dione (14.2 g, 44.893 mmol) in pyridine (99 mL, 1228.96 mmol) was added 1-[chloro-(4-methoxyphenyl)-phenylmethyl]-4-methoxybenzene (18.25 g, 53.871 mmol) at room temperature. Upon completion, as monitored by UPLC-MS, saturated NaHCO₃ (80 mL) was added to the mixture, extracted with EtOAc (200 mL×2), and purified by a silica-gel column chromatography (100 g, Star silica, EtOAc/Hept 10 to 100%) to give 1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methylpyrimidine-2,4(1H,3H)-dione (25 g, 40.408 mmol) in 90% yield.

¹H NMR (400 MHz, DMSO-d₆) δ 11.37 (s, 1H), 7.49 (s, 1H), 7.39 (d, J=7.6 Hz, 2H), 7.35-7.21 (m, 8H), 6.90 (d, J=8.8 Hz, 4H), 5.85 (d, J=4.8 Hz, 1H), 5.12 (d, J=6.0 Hz, 1H), 4.23 (q, J=5.2 Hz, 1H), 4.09 (t, J=4.8 Hz, 1H), 4.02-3.95 (m, 1H), 3.79-3.67 (m, 8H), 3.48 (t, J=4.7 Hz, 2H), 3.26-3.20 (m, 5H), 1.40 (s, 3H).

Step 2: To an aqueous solution of Na₂CO₃ (242 mL, 121.225 mmol) were added 1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methylpyrimidine-2,4(1H,3H)-dione (25 g, 40.408 mmol) in DCM (250 mL, 3885.69 mmol), Tetrabutylammoniumhydrogensulfate (5.49 g, 16.163 mmol), and chloromethyl pivalate (7.30 g, 48.49 mmol) at room temperature. The reaction mixture was stirred at room temperature for 16 h. Some starting material remained unreacted by UPLC-Mass analysis, thus, added 700 mg of chloromethyl pivalate at room temperature. After stirring at rt for another 2 days, the mixture was worked up with saturated NaHCO₃ (50 mL) and extracted with EtOAc (100 mL×3), and purified by a column chromatography (100 g snap, EtOAc/Hept 10 to 60%) to give (3-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-2,6-dioxo-3,6-dihydropyrimidin-1(2H)-yl)methyl pivalate (23 g, 31.4 mmol, 78% yield) along with recovered starting material (3.25 g).

¹H NMR (400 MHz, DMSO-d₆) δ 7.62 (s, 1H), 7.40 (d, J=7.6 Hz, 2H), 7.36-7.20 (m, 7H), 6.90 (d, J=8.8 Hz, 4H), 5.89 (d, J=4.8 Hz, 1H), 5.84-5.73 (m, 2H), 5.17 (d, J=6.0 Hz, 1H), 4.26 (q, J=5.6 Hz, 1H), 4.12 (t, J=4.8 Hz, 1H), 4.02-3.98 (m, 1H), 3.78-3.70 (m, 8H), 3.51-3.40 (m, 2H), 3.28-3.20 (m, 5H), 1.44 (s, 3H), 1.10 (s, 9H); MS (ESI, m/z) calculated for [C₄₀H₄₈N₂O₁₁+Na⁺] 755.32. found 755.1.

### 2′-O-MOE-3′-PSI Activated Monomers

### General Procedure 2¹: PSI Activation

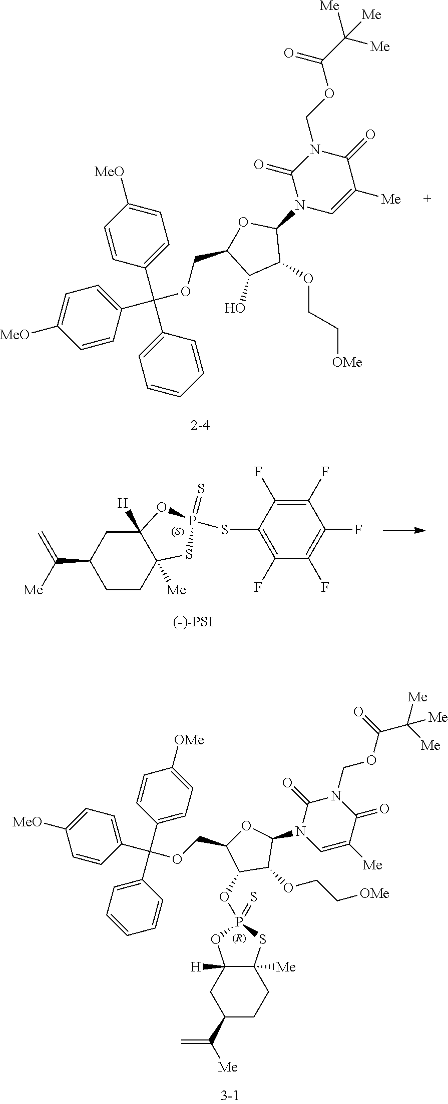

### (Compound 3-1) (3-((2R,3R,4R,5R)-5-((bis(4-3a-methyl-6-(prop-1-en-2-yl)-2-sulfidohexahydrobenzo[d][1,3,2]oxathiaphosphol-2-yl)oxy)tetrahydrofuran-2-yl)-5-methyl-2,6-dioxo-3,6-dihydropyrimidin-1(2H)-yl)methyl Pivalate

(2S,3aS,6R,7aS)-3a-methyl-2-((perfluorophenyl)thio)-6-(prop-1-en-2-yl)hexahydrobenzo[d][1,3,2]oxathiaphosphole 2-sulfide (3.70 g, 8.29 mmol) ((−)-PSI reagent) and (3-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-2,6-dioxo-3,6-dihydropyrimidin-1(2H)-yl)methyl pivalate (4.50 g, 6.141 mmol) were dissolved in THF (20.47 mL, 6.141 mmol) and acetonitrile (20.47 mL, 6.141 mmol), and the solution was cooled in ice bath. To the mixture was added DBU (1.203 mL, 7.983 mmol) and it was stirred at 0 °C until the reaction was completed (0.5-2 h) as monitored by UPLC-MS. The reaction mixture was diluted by EtOAc was washed with saturated NaH₂PO₄ (aq.) solution, then saturated NaHCO₃ (aq.), dried over Na₂SO₄, and purified by a silica gel chromatography (50 g Star, Hept: EtOAc gradient to 70% to give 3-1 as a white solid (5.3 g, 88% yield).

¹H NMR (400 MHz, CD₃CN) δ ppm 7.46-7.54 (3H, m), 7.33-7.39 (6H, m), 7.26-7.32 (1H, m), 6.91 (4H, d, J=8.75 Hz), 5.97 (1H, d, J=6.38 Hz), 5.86-5.93 (2H, m), 5.45-5.52 (1H, m), 5.02 (1H, s), 4.93 (1H, s), 4.45-4.54 (2H, m), 4.26 (1H, d, J=2.88 Hz), 3.77-3.84 (8H, m), 3.47-3.62 (2H, m), 3.42 (1H, dd, J=11.01, 2.88 Hz), 3.29-3.33 (1H, m), 3.28 (3H, s), 2.64 (1H, br s), 2.25-2.32 (1H, m), 2.12-2.14 (3H, m), 2.07 (1H, br dd, J=13.70, 4.44 Hz), 1.99-1.99 (1H, m), 1.81-1.95 (2H, m), 1.80 (3H, s), 1.69 (3H, s), 1.44 (3H, s), 1.18 (9H, s); ³¹P NMR (162 MHz, CD₃CN) δ ppm 101.69; MS (ESI, m/z) calculated for [C₅₀H₆₃N₂O₁₂PS₂+Na⁺] 1001.35. found 1001.4.

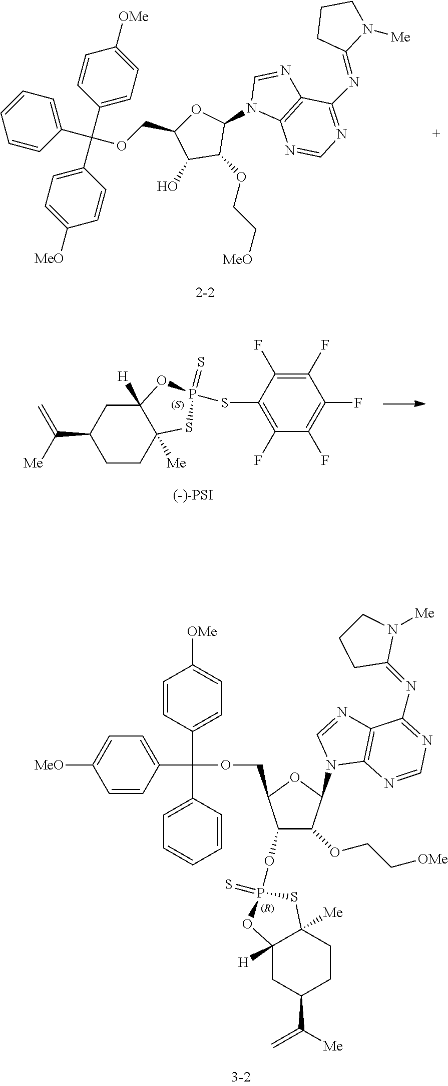

### (Compound 3-2) (2R,3aS,6R,7aS)-2-(((2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-yl)oxy)-3a-methyl-6-(prop-1-en-2-yl)hexahydrobenzo[d][1,3,2]oxathiaphosphole 2-sulfide

Prepared according to general procedure 2 with (−)-PSI reagent, 78% yield; foamy solid; ¹H NMR (400 MHz, CDCl₃) δ 8.40-8.46 (m, 1H), 8.00-8.03 (m, 1H), 7.36-7.41 (m, 2H), 7.24-7.30 (m, 4H), 7.17-7.22 (m, 2H), 7.08-7.16 (m, 1H), 6.69-6.78 (m, 4H), 6.05 (d, J=7.5 Hz, 1H), 5.45-5.59 (m, 1H), 5.04 (dd, J=7.5, 4.7 Hz, 1H), 4.95 (s, 1H), 4.78-4.93 (m, 1H), 4.50 (dt, J=12.6, 3.3 Hz, 1H), 4.27-4.33 (m, 1H), 3.59-3.79 (m, 10H), 3.31-3.46 (m, 5H), 3.10-3.15 (m, 3H), 3.06-3.10 (m, 3H), 2.83-2.97 (m, 2H), 2.47-2.54 (m, 1H), 2.16-2.24 (m, 1H), 2.03-2.13 (m, 1H), 1.94-2.03 (m, 2H), 1.74-1.94 (m, 4H), 1.66-1.69 (m, 3H), 1.60-1.65 (m, 3H); 13C NMR (101 MHz, CDCl₃) δ 166.9, 160.9, 158.6, 158.5, 152.8, 151.6, 144.8, 144.5, 140.1, 135.6, 135.6, 130.2, 130.1, 128.2, 128.0, 126.9, 126.6, 113.3, 112.2, 86.8, 85.4, 85.4, 83.6, 83.5, 80.1, 80.0, 77.3, 76.9, 72.3, 70.6, 68.0, 65.7, 63.0, 58.9, 55.2, 51.6, 38.9, 33.7, 33.7, 32.0, 30.1, 27.8, 27.6, 25.6, 23.5, 22.7, 21.8, 19.7; ³¹P NMR (162 MHz, CDCl₃) δ 101.34; MS (ESI, m/z) calculated for [C₄₉H₅₉N₆O₈PS₂+H⁺]955.36. found 956.3.

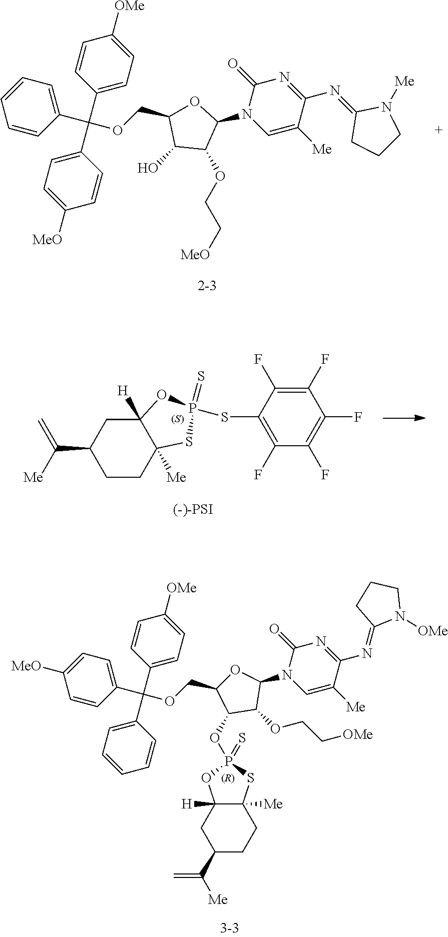

### (Compound 3-3) 1-((2R,3R,4R,5R)-5-((bis(4-methoxyphenyl)(phenyl)methoxy)methyl)-3-(2-methoxyethoxy)-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-5-methyl-4-((1-methylpyrrolidin-2-ylidene)amino)pyrimidin-2(1H)-one

Prepared according to general procedure 2 with (−)-PSI reagent, foamy solid, 70% yield; 1H NMR (400 MHz, CD₃CN) δ ppm 7.59 (1H, s), 7.50 (2H, d, J=7.38 Hz), 7.32-7.41 (6H, m), 7.25-7.31 (1H, m), 6.90 (4H, d, J=8.63 Hz), 5.99 (1H, d, J=5.00 Hz), 5.44 (1H, dt, J=12.98, 5.02 Hz), 5.02 (1H, s), 4.93 (1H, s), 4.47 (1H, dt, J=12.73, 3.20 Hz), 4.34 (1H, t, J=5.07 Hz), 4.22-4.28 (1H, m), 3.86-3.94 (1H, m), 3.79 (6H, s), 3.44-3.61 (4H, m), 3.33-3.41 (3H, m), 3.30 (3H, s), 3.05-3.09 (1H, m), 3.04 (3H, s), 2.76 (1H, s), 2.64 (1H, br s), 2.20-2.30 (2H, m), 2.01-2.09 (3H, m), 1.99-1.99 (2H, m), 1.81-1.92 (1H, m), 1.80 (3H, s), 1.68 (3H, s), 1.55 (3H, s); ³¹P NMR (162 MHz, CD₃CN) δ ppm 101.51; MS (ESI, m/z) calculated for [C₄₉H₆₁N₄O₉PS₂+H⁺] 945.36. found 945.4.

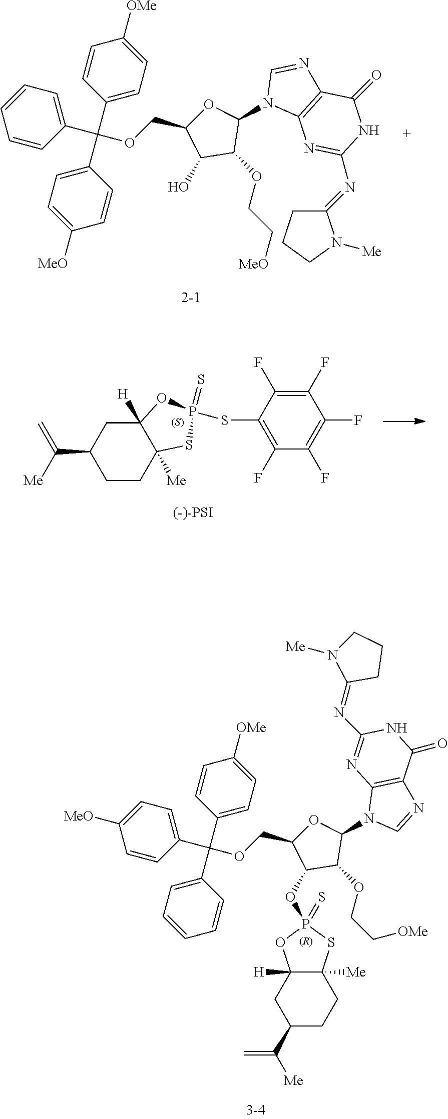

### (Compound 3-4) 9-((2R,3R,4R,5R)-5-((bis(4-methoxyphenyl)(phenyl)methoxy)methyl)-3-(2-methoxyethoxy)-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-2-((−1-methylpyrrolidin-2-ylidene)amino)-1,9-dihydro-6H-purin-6-one

Prepared according to general procedure 2 with (−)-PSI reagent, foamy solid, 80% yield; ¹H NMR (400 MHz, CD₃CN) δ ppm 9.18 (1H, br s), 7.74 (1H, s), 7.43 (2H, d, J=7.38 Hz), 7.22-7.33 (7H, m), 6.85 (4H, dd, J=9.01, 2.63 Hz), 5.89 (1H, d, J=5.50 Hz), 5.48 (1H, dt, J=13.54, 4.80 Hz), 4.98 (1H, s), 4.92 (1H, s), 4.85 (1H, t, J=5.38 Hz), 4.50 (1H, dt, J=12.69, 3.22 Hz), 4.23 (1H, q, J=4.09 Hz), 3.79 (6H, s), 3.67-3.77 (2H, m), 3.43-3.51 (4H, m), 3.32 (2H, qd, J=10.94, 4.06 Hz), 3.21 (3H, s), 3.03 (3H, s), 2.99-3.02 (1H, m), 2.63 (1H, br s), 2.27 (1H, br d, J=13.13 Hz), 2.12-2.15 (1H, m), 2.01-2.06 (1H, m), 1.99-1.99 (4H, m), 1.79-1.93 (2H, m), 1.77 (3H, s), 1.68 (3H, s); ³¹P NMR (162 MHz, CD₃CN) δ ppm 101.36; MS (ESI, m/z) calculated for [C₄₉H₅₉N₆O₉PS₂+H⁺] 971.35. found 971.4.

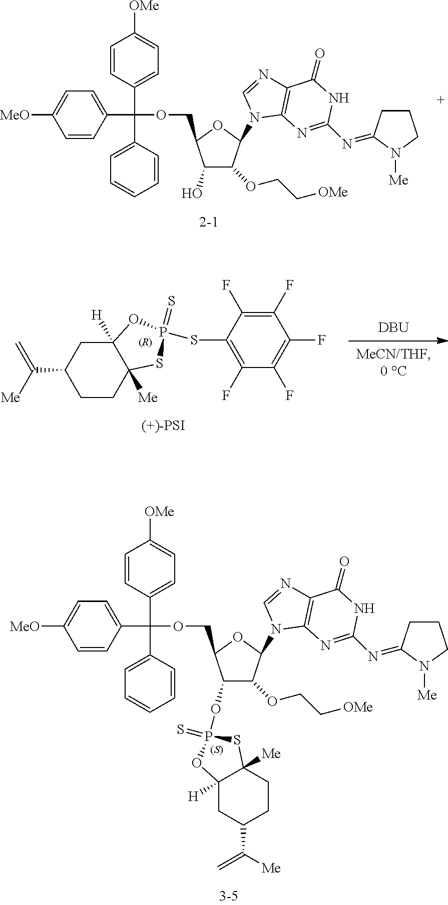

### (Compound 3-5) 9-((2R,3R,4R,5R)-5-((bis(4-methoxyphenyl)(phenyl)methoxy)methyl)-3-(2-methoxyethoxy)-4-(((2S,3aR,6S,7aR)-3a-methyl-6-(prop-1-en-2-yl)-2-sulfidohexahydrobenzo[d][1,3,2]oxathiaphosphol-2-yl)oxy)tetrahydrofuran-2-yl)-2-((1-methylpyrrolidin-2-ylidene)amino)-1,9-dihydro-6H-purin-6-one

Prepared according to general procedure 2 with (+)-PSI reagent, white foamy solid; ¹H NMR (400 MHz, CD₃CN, 296 K) δ (ppm)=9.56 (br s, 1H), 7.74 (s, 1H), 7.44 (d, J=7.5 Hz, 2H), 7.34-7.28 (m, 6H), 7.28-7.21 (m, 1H), 6.86 (dd, J=2.4, 8.9 Hz, 4H), 5.89 (d, J=6.4 Hz, 1H), 5.44-5.36 (m, 1H), 4.99 (s, 1H), 4.89 (s, 1H), 4.78 (t, J=5.8 Hz, 1H), 4.45 (td, J=3.0, 12.7 Hz, 1H), 4.27 (q, J=3.9 Hz, 1H), 3.78 (s, 6H), 3.75-3.70 (m, 1H), 3.67-3.57 (m, 1H), 3.47-3.40 (m, 2H), 3.39-3.32 (m, 4H), 3.12 (s, 3H), 3.09-2.92 (m, 5H), 2.63 (br s, 1H), 2.30-2.15 (m, 2H), 2.04 (br dd, J=4.0, 12.9 Hz, 1H), 2.00-1.90 (m, 4H), 1.82 (br s, 1H), 1.79-1.75 (m, 3H), 1.68 (s, 3H); ¹³C NMR (101 MHz, CD₃CN, 298 K) δ (ppm)=170.8, 160.1, 159.3, 158.3, 152.1, 147.2, 146.2, 137.9, 136.9, 136.9, 131.5, 131.4, 129.4, 129.3, 128.3, 114.5, 112.4, 87.9, 87.6, 87.3, 83.5, 83.4, 81.8, 78.2, 78.1, 73.1, 72.3, 66.4, 64.4, 59.4, 56.3, 52.4, 40.2, 34.9, 34.8, 32.5, 32.4, 28.7, 28.6, 24.2, 23.2, 22.3, 20.8; ³¹P NMR (162 MHz, CD₃CN) δ 101.9; MS (ESI, m/z) calculated for [C₄₉H₅₉N₆O₉PS₂+H⁺] 971.35. found 971.1.

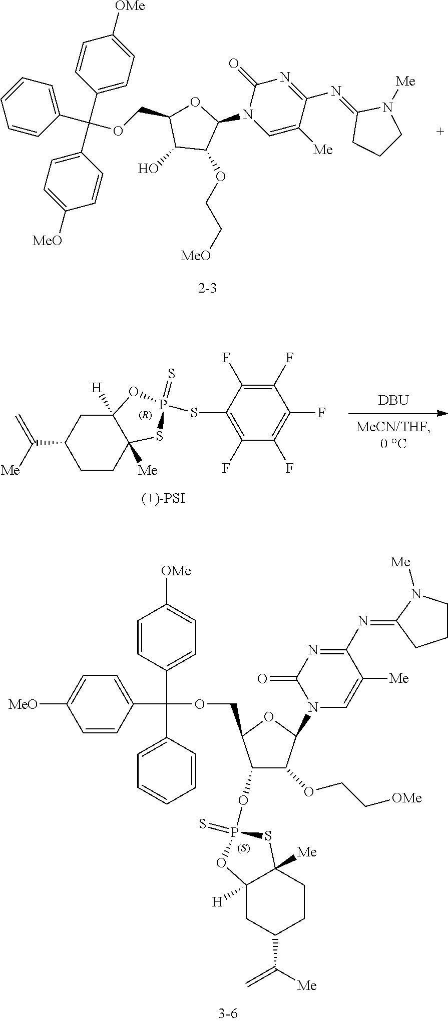

(Compound 3-6) 1-((2R,3R,4R,5R)-5-((bis(4-methoxyphenyl)(phenyl)methoxy)methyl)-3-(2-methoxyethoxy)-4-(((2S,3aR,6S,7aR)-3a-methyl-6-(prop-1-en-2-yl)-2-sulfidohexahydrobenzo[d][1,3,2]oxathiaphosphol-2-yl)oxy)tetrahydrofuran-2-yl)-5-methyl-4-(((E)-1-methylpyrrolidin-2-ylidene)amino)pyrimidin-2(1H)-one: Prepared according to general procedure 2 with (+)-PSI reagent, white foamy solid; ¹H NMR (400 MHz, CD₃CN, 296 K) δ (ppm)=7.57 (s, 1H), 7.50 (d, J=7.5 Hz, 2H), 7.40-7.31 (m, 6H), 7.31-7.23 (m, 1H), 6.90 (d, J=8.9 Hz, 4H), 6.05 (d, J=5.8 Hz, 1H), 5.49-5.40 (m, 1H), 4.99 (s, 1H), 4.88 (s, 1H), 4.43 (td, J=3.1, 12.6 Hz, 1H), 4.34 (t, J=5.4 Hz, 1H), 4.26 (br d, J=3.4 Hz, 1H), 3.85-3.72 (m, 8H), 3.54-3.45 (m, 4H), 3.38 (d, J=2.8 Hz, 2H), 3.26 (s, 3H), 3.11-3.05 (m, 2H), 3.03 (s, 4H), 2.62 (br s, 1H), 2.22 (br d, J=12.3 Hz, 1H), 2.13-2.01 (m, 3H), 2.01-1.92 (m, 2H), 1.92-1.79 (m, 2H), 1.76 (s, 3H), 1.68 (s, 3H), 1.56 (s, 3H); ¹³C NMR (101 MHz, CD₃CN, 298 K) δ (ppm)=172.6, 170.0, 160.2, 147.2, 146.1, 138.4, 137.0, 136.8, 131.5, 131.5, 129.5, 129.4, 128.4, 114.6, 112.5, 88.7, 88.2, 87.8, 82.9, 82.9, 82.3, 82.2, 77.7, 77.6, 73.3, 71.8, 66.7, 63.9, 59.5, 56.3, 52.5, 40.2, 34.9, 34.8, 32.4, 31.8, 28.7, 28.6, 24.2, 23.2, 22.3, 20.8; ³¹P NMR (162 MHz, CD₃CN) δ 101.8; MS (ESI, m/z) calculated for [C₄₉H₆₁N₄O₉PS₂+H⁺] 945.36. found 946.5.

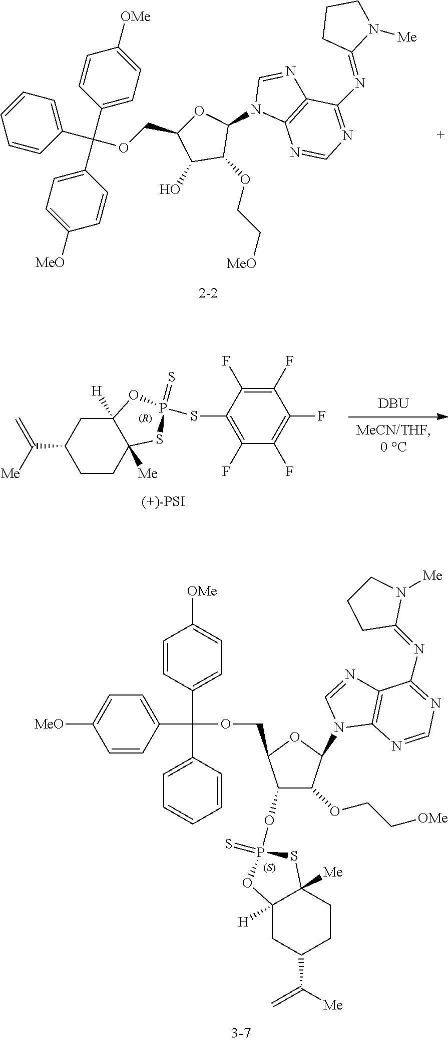

### (Compound 3-7) (2S,3aR,6S,7aR)-2-(((2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-yl)oxy)-3a-methyl-6-(prop-1-en-2-yl)hexahydrobenzo[d][1,3,2]oxathiaphosphole 2-sulfide

Prepared according to general procedure 2 with (+)-PSI reagent, white foamy solid; ¹H NMR (400 MHz, CD₃CN, 296 K) δ (ppm)=8.35 (s, 1H), 8.06 (s, 1H), 7.47 (d, J=7.4 Hz, 2H), 7.34 (dd, J=1.6, 8.8 Hz, 4H), 7.30 (s, 2H), 7.26-7.19 (m, 1H), 6.85 (d, J=8.9 Hz, 4H), 6.01 (d, J=6.6 Hz, 1H), 5.60-5.53 (m, 1H), 5.10 (t, J=5.7 Hz, 1H), 4.99 (s, 1H), 4.91 (s, 1H), 4.49 (td, J=3.0, 12.6 Hz, 1H), 4.41-4.32 (m, 1H), 3.78 (s, 6H), 3.77-3.71 (m, 1H), 3.67 (br t, J=6.4 Hz, 2H), 3.65-3.57 (m, 1H), 3.52 (t, J=7.1 Hz, 2H), 3.45 (br d, J=4.5 Hz, 1H), 3.40-3.34 (m, 3H), 3.09 (s, 3H), 3.07 (s, 3H), 2.93 (t, J=7.9 Hz, 2H), 2.64 (br s, 1H), 2.27 (br d, J=13.4 Hz, 1H), 2.18-2.06 (m, 1H), 2.06-2.01 (m, 2H), 2.00-1.86 (m, 1H), 1.78 (s, 3H), 1.70 (s, 3H); ¹³C NMR (101 MHz, CD₃CN, 297 K) δ (ppm)=168.5, 162.1, 160.1, 153.5, 152.6, 147.2, 146.3, 142.2, 137.0, 131.5, 131.5, 129.4, 129.2, 129.2, 128.3, 128.0, 114.5, 112.5, 87.8, 87.7, 87.6, 83.9, 83.8, 80.8, 80.8, 78.4, 78.4, 72.9, 72.1, 68.7, 66.6, 64.2, 59.3, 56.3, 52.5, 40.2, 34.9, 34.8, 32.4, 31.3, 28.7, 28.6, 26.6, 24.2, 23.2, 22.3, 20.8; ³¹P NMR (162 MHz, CD₃CN) δ 101.7; MS (ESI, m/z) calculated for [C₄₉H₅₉N₆O₈PS₂+H⁺] 955.36. found 956.6.

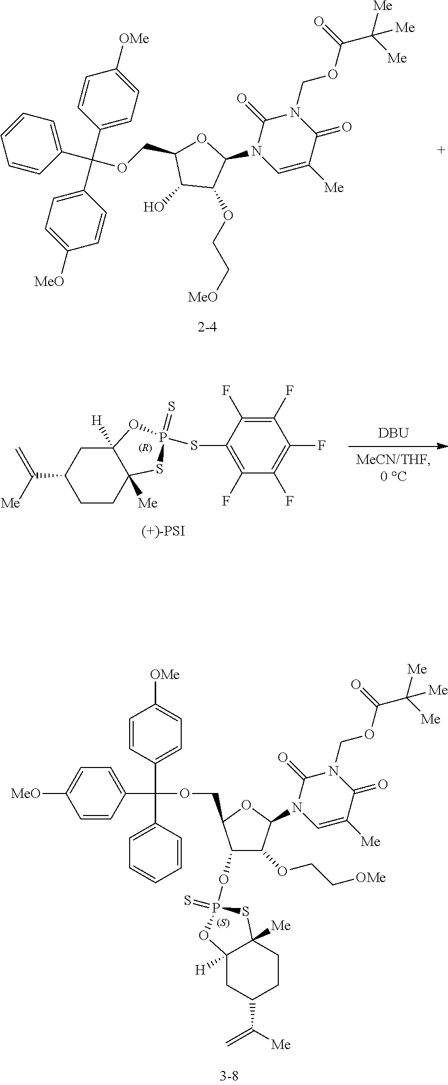

### (Compound 3-8) (3-((2R,3R,4R,5R)-5-((bis(4-methoxyphenyl) (phenyl) methoxy)methyl)-3-(2-methoxyethoxy)-4-(((2S,3aR,6S,7aR)-3a-methyl-6-(prop-1-en-2-yl)-2-sulfidohexahydrobenzo[d] [1,3,2]oxathiaphosphol-2-yl)oxy)tetrahydrofuran-2-yl)-5-methyl-2,6-dioxo-3,6-dihydropyrimidin-1 (2H)-yl)methyl Pivalate

Prepared according to general procedure 2 with (+)-PSI reagent, foamy solid; ¹H NMR (400 MHz, CD₃CN) δ ppm 7.53 (1H, s), 7.48 (2H, d, J=7.63 Hz), 7.33-7.39 (6H, m), 7.26-7.32 (1H, m), 6.92 (4H, d, J=8.76 Hz), 6.01 (1H, d, J=6.88 Hz), 5.86-5.92 (2H, m), 5.45 (1H, ddd, J=11.60, 4.78, 2.75 Hz), 5.01 (1H, s), 4.90 (1H, s), 4.44-4.49 (2H, m), 4.29 (1H, br d, J=2.63 Hz), 3.80 (6H, s), 3.76-3.79 (1H, m), 3.32-3.54 (4H, m), 3.23 (3H, s), 2.60-2.66 (1H, m), 2.24 (2H, br d, J=12.76 Hz), 2.07 (1H, br dd, J=13.01, 3.75 Hz), 1.99-2.01 (2H, m), 1.80-1.92 (2H, m), 1.78 (3H, s), 1.68 (3H, s), 1.46 (3H, s), 1.18 (9H, s); 31P NMR (162 MHz, CD₃CN) δ ppm 102.18; MS (ESI, m/z) calculated for [C₅₀H₆₃N₂O₁₂PS₂+Na⁺] 1001.35. found 1001.1.

General Procedure 3: PO-PSI Monomer from PS-PSI Monomer

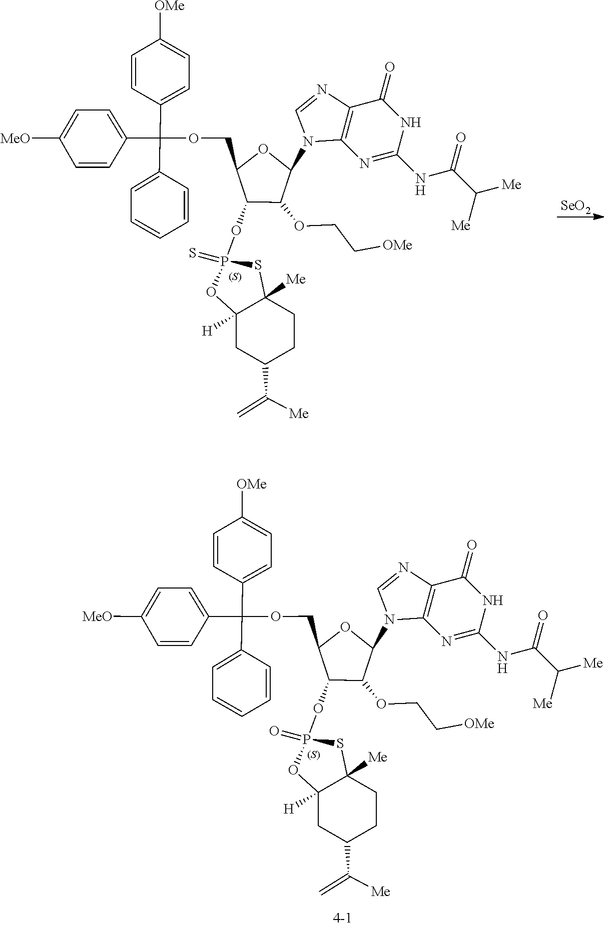

### (Compound 4-1) N-(9-((2R,3R,4R,5R)-5-((bis(4-methoxyphenyl)(phenyl)methoxy)methyl)-3-(2-methoxyethoxy)-4-(((2S,3aR,6S,7aR)-3a-methyl-2-oxido-6-(prop-1-en-2-yl)hexahydrobenzo[d][1,3,2]oxathiaphosphol-2-yl)oxy)tetrahydrofuran-2-yl)-6-oxo-6,9-dihydro-1H-purin-2-yl)isobutyramide

To N-(9-((2R,3R,4R,5R)-5-((bis(4-methoxyphenyl)(phenyl)methoxy)methyl)-3-(2-methoxyethoxy)-4-(((2S,3aR,6S,7aR)-3a-methyl-6-(prop-1-en-2-yl)-2-sulfidohexahydrobenzo[d][1,3,2]oxathiaphosphol-2-yl)oxy)tetrahydrofuran-2-yl)-6-oxo-6,9-dihydro-1H-purin-2-yl)isobutyramide (1 g, 1.042 mmol) in MeCN (15.00 mL, 15 vol) was added SeO₂ (1.0 eq., 0.116 g, 1.042 mmol) in ice-bath. Additional SeO₂ (0.116 g, 1.042 mmol) was added at 0 °C until the reaction was completed at 0 °C. Total 3 equivalents of SeO₂ were used. Upon completion as monitored by UPLC-MS, the mixture was filtered over celite and dry SiO₂ (EtOAc/THF). The filtrate was washed with saturated NaHCO₃ (10 mL), dried over Na₂SO₄, filtered (dry SiO₂) and concentrated. The residue was purified by a silica-gel column chromatography (50 g, Hept/EtOAc, 20 to 100 then EtOAc/THF 0 to 100%) to give the 4-1 (0.55 g, 56% yield). MS (ESI, m/z) calculated for [C₄₈H₅₈N₅O₁₁PS—H⁺] 942.36. found 942.53.

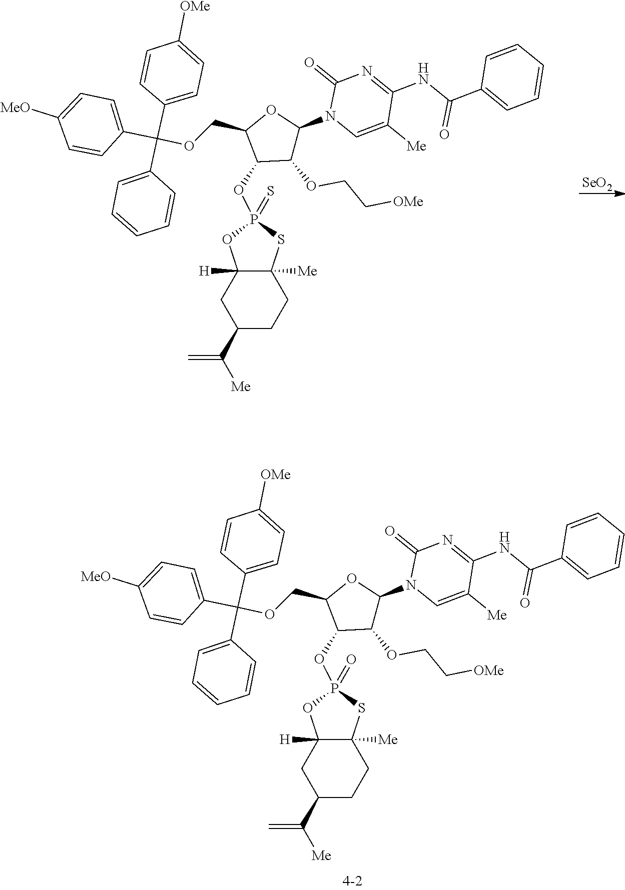 [SMILES: C=C(C)[C@@H]1CC[C@]2(C)S[P@](=S)(O[C@H]3[C@@H](OCCOC)[C@H](n4cc(C)c(NC(=O)c5ccccc5)nc4=O)O[C@@H]3COC(c3ccccc3)(c3ccc(OC)cc3)c3ccc(OC)cc3)O[C@H]2C1 ; "SeO2" ; C=C(C)[C@@H]1CC[C@]2(C)S[P@](=O)(O[C@H]3[C@@H](OCCOC)[C@H](n4cc(C)c(NC(=O)c5ccccc5)nc4=O)O[C@@H]3COC(c3ccccc3)(c3ccc(OC)cc3)c3ccc(OC)cc3)O[C@H]2C1]

### (Compound 4-2) N-(1-((2R,3R,4R,5R)-5-((bis(4-methoxyphenyl)(phenyl)methoxy)methyl)-3-(2-methoxyethoxy)-4-(((2R,3aS,6R,7aS)-3a-methyl-2-oxido-6-(prop-1-en-2-yl)hexahydrobenzo[d][1,3,2]oxathiaphosphol-2-yl)oxy)tetrahydrofuran-2-yl)-5-methyl-2-oxo-1,2-dihydropyrimidin-4-yl)benzamide

Prepared according to general procedure 3, white foamy solid; 59% yield; ³¹P NMR (162 MHz, acetonitrile-d₃) δ 40.36; MS (ESI, m/z) calculated for [C₅₁H₅₈N₃O₁₁PS+H⁺] 952.35. found 952.35.

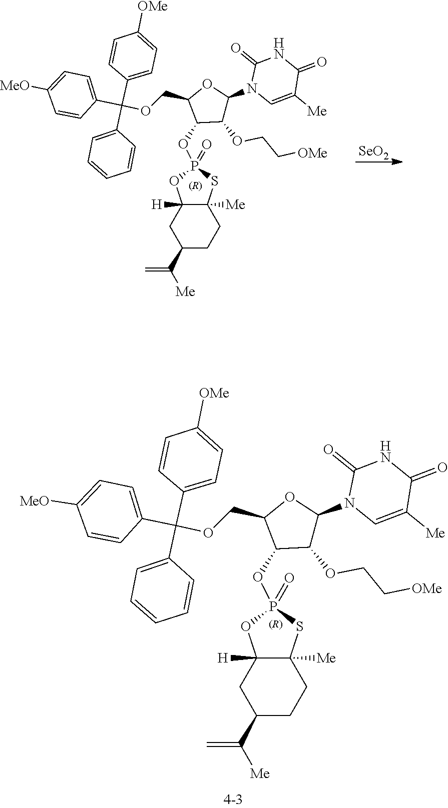

### (Compound 4-3) 1-((2R,3R,4R,5R)-5-((bis(4-methoxyphenyl)(phenyl)methoxy)methyl)-3-(2-methoxyethoxy)-4-(((2R,3aS,6R,7aS)-3a-methyl-2-oxido-6-(prop-1-en-2-yl)hexahydrobenzo[d][1,3,2]oxathiaphosphol-2-yl)oxy)tetrahydrofuran-2-yl)-5-methylpyrimidine-2,4(1H,3H)-dione

Prepared according to general procedure 3, white foamy solid; 46% yield; ³¹P NMR (162 MHz, acetonitrile-d₃) δ 40.42; MS (ESI, m/z) calculated for [C₄₄H₅₃N₂O₁₁PS+Na⁺]871.30. found 871.28.

PO-PSI Reagent from Cyclohexyl Epoxide:

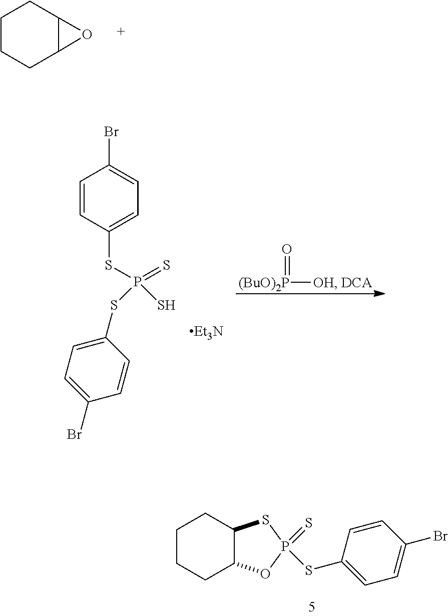

### (Compound 5) rac-2-((4-bromophenyl)thio)hexahydrobenzo[d][1,3,2]oxathiaphosphole 2-sulfide

A solution of triethylamine bis(4-bromophenyl) phosphorotetrathioate (50.0 g, 87.2 mmol) and cyclohexene oxide (13.2 mL, 131 mmol) in chloroform (175 mL) was treated with dibutyl phosphate (16.2 mL, 87.2 mmol) and dichloroacetic acid (10.8 mL, 131 mmol). After stirring at room temperature for 15 hours, the mixture was concentrated in vacuo. The residue was diluted with water (125 mL) and n-heptane (125 mL), cooled with an ice bath, and stirred at 0 °C for 2 hours. The resulting precipitate was filtered, and washed subsequently with water (100 mL) and n-heptane (125 mL). The filter cake was dissolved in CH₂Cl₂ (200 mL) and the aqueous layer was removed. The organic layer was concentrated in vacuo to ca. 50 mL and treated with n-heptane (75 mL). The mixture was stirred at room temperature for 20 min and concentrated in vacuo to ca. 50 mL. The resulting precipitate was filtered, washed with n-heptane (20 mL), and dried over N2 purge for 2 hours to give the title compound (30.1 g, 91%).

¹H NMR (400 MHz, CDCl₃, 296 K) (a 1:2 mixture of diastereomers) δ (ppm)=7.58-7.51 (m, 8H), 7.47-7.41 (m, 4H), 4.04 (dt, J=3.9, 10.7 Hz, 1H), 3.65-3.56 (m, 4H), 2.27-2.12 (m, 6H), 1.89 (m, 3H), 1.81 (m, 3H), 1.75-1.58 (m, 3H), 1.49-1.25 (m, 8H), 1.23-1.16 (m, 1H), 1.07-0.86 (m, 1H); ³¹P NMR (162 MHz, CDCl₃, 296 K) δ (ppm)=107.01 (s, 1P), 103.23 (s, 2P); MS (ESI) m/z: [M+H]⁺ calcd for C₁₂H₁₅BrOPS₃ 380.91. Found 380.84.

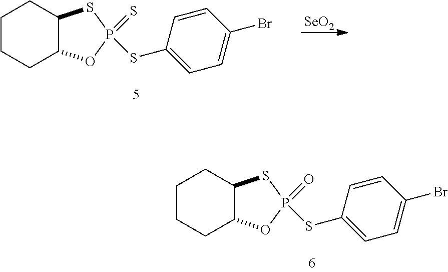

### (Compound 6) rac-2-((4-bromophenyl)thio)hexahydrobenzo[d][1,3,2]oxathiaphosphole 2-oxide

A solution of (3aR,7aR)-2-((4-bromophenyl)thio)hexahydrobenzo[d][1,3,2]oxathiaphosphole 2-sulfide (10.0 g, 26.2 mmol) in CH₂Cl₂ (170 mL) was treated with SeO₂ (2.91 g, 26.2 mmol) and stirred at room temperature for 2 hours. Additional SeO₂ (2.91 g, 26.2 mmol) was added and stirring was continued at rt for additional 19 hours. The reaction mixture was filtered through a dry silica gel pad and rinsed with CH₂Cl₂. The filtrate was washed with 10% NaH₂PO₄ (70.0 mL), dried over MgSO₄ and concentrated in vacuo. The residue was treated with n-heptane (46 mL) and the resulting slurry was stirred at room temperature for 20 minutes. The precipitate was filtered, washed with n-heptane (20 mL) and dried over N₂ purge to give the title compound (6.18 g, 64.5%).

¹H NMR (400 MHz, CDCl₃, 296 K) (ca. 1:2 mixture of two diastereomers) δ (ppm)=7.58-7.48 (m, 12H), 4.10 (dt, J=4.1, 10.8 Hz, 1H), 3.60 (dt, J=3.6, 10.8 Hz, 2H), 3.37 (dt, J=3.9, 10.8 Hz, 2H), 2.43-2.36 (m, 1H), 2.25-2.07 (m, 5H), 1.98-1.83 (m, 4H), 1.83-1.73 (m, 3H), 1.63-1.48 (m, 3H), 1.46-1.23 (m, 8H), 1.11-0.99 (m, 1H); ³¹P NMR (162 MHz, CDCl₃, 297 K) δ (ppm)=62.54 (s, 1P), 56.98 (s, 2P); MS (ESI) m/z: [M+H]⁺ calcd for C₁₂H₁₅BrO₂PS₂ 364.94. Found 364.97.

### General Procedure 4: PO-PSI Monomer

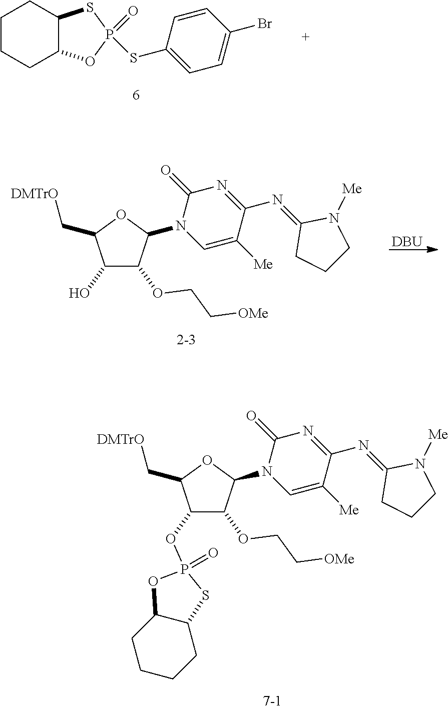

### (Compound 7-1) 1-((2R,3R,4R,5R)-5-((bis(4-methoxyphenyl)(phenyl)methoxy)methyl)-3-(2-methoxyethoxy)-4-(((3aR,7aR)-2-oxidohexahydrobenzo[d][1,3,2]oxathiaphosphol-2-yl)oxy)tetrahydrofuran-2-yl)-5-methyl-4-(((E)-1-methylpyrrolidin-2-ylidene)amino)pyrimidin-2(1H)-one

1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-4-(((E)-1-methylpyrrolidin-2-ylidene)amino)pyrimidin-2(1H)-one (4.30 g, 6.15 mmol) and (3aR,7aR)-2-((4-bromophenyl)thio)hexahydrobenzo[d][1,3,2]oxathiaphosphole 2-oxide (3.15 g, 8.62 mmol) was azeotroped three times with acetonitrile (43 mL). The residue was dissolved in acetonitrile (43 mL), cooled to 0 °C, and treated with DBU (1.6 mL, 8.3 mmol). The mixture was stirred at 0 °C for 2 hours, quenched with saturated NaH₂PO₄ (40 mL), and diluted with ethyl acetate (50 mL). The organic layer was separated, and the aqueous layer was extracted twice with ethyl acetate (50 mL). The organic layers were combined, washed with sat. NaHCO₃ (20 mL), dried over MgSO₄, and concentrated in vacuo. The residue was purified by silica gel column chromatography (ethyl acetate in n-heptane=17% to 100% and then THF in ethyl acetate=0% to 100%) to give the title compound (3.07 g, 57.1%) as a foaming solid.

MS (ESI) m/z: [M+H]⁺ calcd for C₄₅H₅₆N₄O₁₀PS 875.3. Found 875.1.

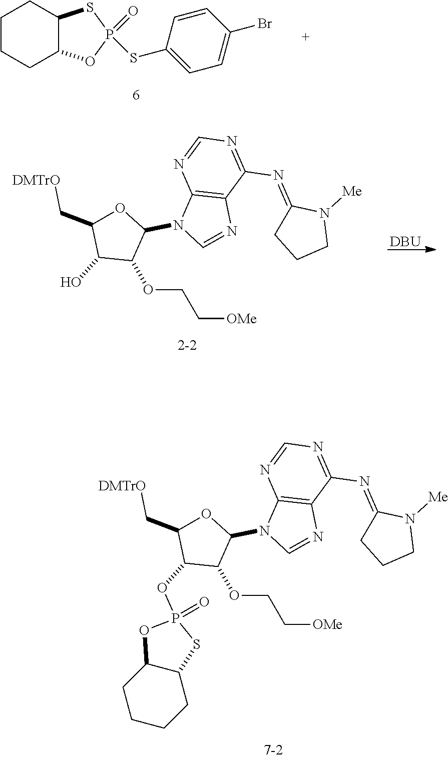

### (Compound 7-2) (3aR,7aR)-2-(((2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-yl)oxy)hexahydrobenzo[d][1,3,2]oxathiaphosphole 2-oxide

(2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-ol (2.70 g, 3.81 mmol) and (3aR,7aR)-2-((4-bromophenyl)thio)hexahydrobenzo[d][1,3,2]oxathiaphosphole 2-oxide (1.95 g, 5.33 mmol) was azeotroped three times with acetonitrile (25.4 mL) in the rotary evaporator. The residue was dissolved in acetonitrile (25.4 mL), cooled to 0 °C, and treated with DBU (0.78 mL, 5.1 mmol). The mixture was stirred at 0 °C for 2 hours, quenched with saturated NaH₂PO₄ (30 mL), and diluted with ethyl acetate (30 mL). The organic layer was separated, and the aqueous layer was extracted twice with ethyl acetate (30 mL). The organic layers were combined, washed with saturated NaHCO₃ (20 mL), dried over MgSO₄, and concentrated in vacuo. The residue was purified by column chromatography (ethyl acetate in n-heptane=17% to 100%, and then THF in ethyl acetate=0% to 100%) to give the title compound (2.10 g, 62.3%) as a foamy solid.

MS (ESI) m/z: [M+H]⁺ calcd for C₄₅H₅₄N₆O₉PS 884.33. Found 884.45.

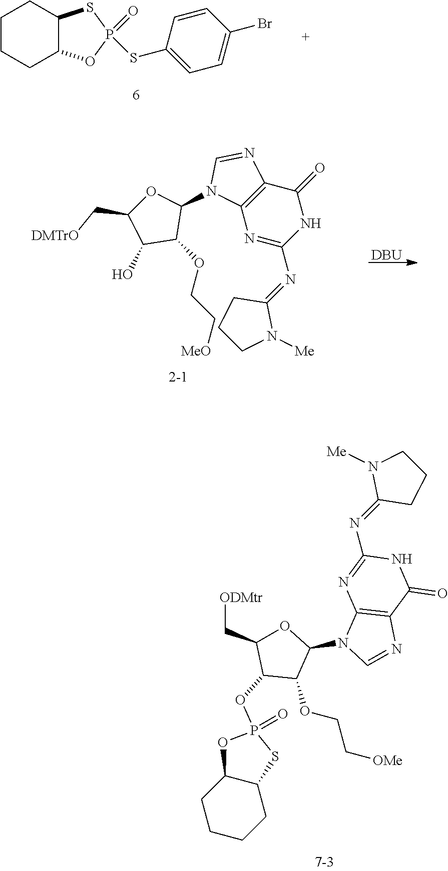

### (Compound 7-3) 9-((2R,3R,4R,5R)-5-((bis(4-methoxyphenyl)(phenyl)methoxy)methyl)-3-(2-methoxyethoxy)-4-(((3aR,7aR)-2-oxidohexahydrobenzo[d][1,3,2]oxathiaphosphol-2-yl)oxy)tetrahydrofuran-2-yl)-2-((1-methylpyrrolidin-2-ylidene)amino)-1,9-dihydro-6H-purin-6-one

Prepared according to general procedure 4, a white foamy solid; 80% yield; MS (ESI, m/z) calculated for [C₄₅H₅₃N₆O₁₀PS+H⁺] 901.33. found 901.1.

### General Procedure 5: Synthesis of Monomer Succinates

To the protected nucleoside (1.0 eq.) and succinic anhydride (1.5 eq.) were added DCM (8 vol) and Et₃N (3.0 eq.) at room temperature. The mixture was stirred overnight at room temperature. To the mixture was added phosphate buffer (pH 7, 6 vol) and extracted with DCM (8 vol) 3 times. Then the organic layers were concentrated and purified by a column chromatography (Heptane/EtOAc, 10 to 100%).

### (5-Methyl-C-MOE Succinate):

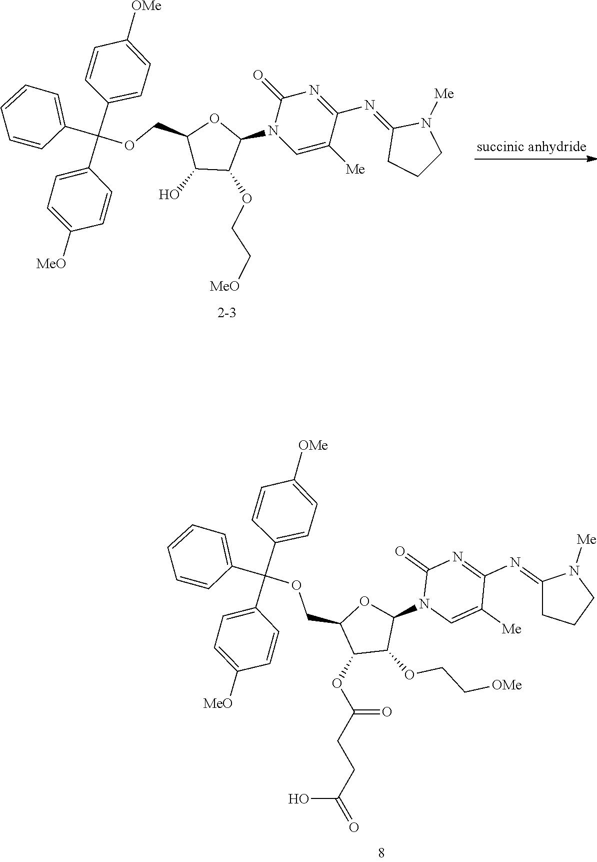 [SMILES: COCCO[C@@H]1[C@H](O)[C@@H](COC(c2ccccc2)(c2ccc(OC)cc2)c2ccc(OC)cc2)O[C@H]1n1cc(C)c(/N=C2\CCCN2C)nc1=O ; COCCO[C@@H]1[C@H](OC(=O)CCC(=O)O)[C@@H](COC(c2ccccc2)(c2ccc(OC)cc2)c2ccc(OC)cc2)O[C@H]1n1cc(C)c(/N=C2\CCCN2C)nc1=O]

### (Compound 8) 4-(((2R,3R,4R,5R)-2-((bis(4-methoxyphenyl)(phenyl)methoxy)methyl)-4-(2-methoxyethoxy)-5-(5-methyl-4-(((E)-1-methylpyrrolidin-2-ylidene)amino)-2-oxopyrimidin-1(2H)-yl)tetrahydrofuran-3-yl)oxy)-4-oxobutanoic Acid

To 1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-4-(((E)-1-methylpyrrolidin-2-ylidene)amino)pyrimidin-2(1H)-one (5 g, 7.155 mmol) and succinic anhydride (1.074 g, 10.732 mmol) in DCM (40.0 mL, 621.71 mmol) was added Et₃N (2.99 mL, 21.465 mmol) at room temperature. The mixture was stirred overnight at room temperature. To the mixture was added phosphate buffer (pH 7, 30 mL) and extracted with DCM (50 mL×3). Then the organic layers were concentrated and purified by a column chromatography (Hept/EtOAc, 10 to 100%) to give 4-(((2R,3R,4R,5R)-2-((bis(4-methoxyphenyl)(phenyl)methoxy)methyl)-4-(2-methoxyethoxy)-5-(5-methyl-4-(((E)-1-methylpyrrolidin-2-ylidene)amino)-2-oxopyrimidin-1(2H)-yl)tetrahydrofuran-3-yl)oxy)-4-oxobutanoic acid (4.92 g, 6.16 mmol, 86% yield).

¹H NMR (400 MHz, CD₃CN) δ ppm 7.59 (1H, s), 7.47 (2H, d, J=7.50 Hz), 7.31-7.39 (6H, m), 7.24-7.31 (1H, m), 6.91 (4H, d, J=8.63 Hz), 6.04 (1H, d, J=5.25 Hz), 5.35 (1H, t, J=5.13 Hz), 4.35 (1H, t, J=5.32 Hz), 4.14-4.25 (1H, m), 3.79 (6H, s), 3.74-3.78 (1H, m), 3.66 (1H, dt, J=11.44, 4.28 Hz), 3.44-3.52 (4H, m), 3.33-3.40 (2H, m), 3.26 (3H, s), 3.05-3.11 (2H, m), 3.04 (3H, s), 2.50-2.65 (4H, m), 2.00-2.08 (2H, m), 1.99 (1H, s), 1.61 (3H, s); MS (ESI, m/z) Calculated for [C₄₃H₅₀N₄O₁₁+H⁺] 799.35. Found 799.9.

### General Procedure 6: Solid Phase Synthesis of Stereo-Controlled PS MOE ASO

A general procedure for automated solid-phase synthesis of stereo-controlled PS-oligonucleotides was modified from the reported procedures in Knouse et al., “Unlocking P(V): Reagents for chiral phosphorothioate synthesis,” Science 2018, 361 (6408), 1234-1238; and Huang et al., “A P(V) platform for oligonucleotide synthesis,” Science 2021, 373 (6560), 1265-1270.

### Automated Solid-Phase Oligonucleotide Synthesis:

### Part 1. Loading to Resin: Preparation of 1Mer

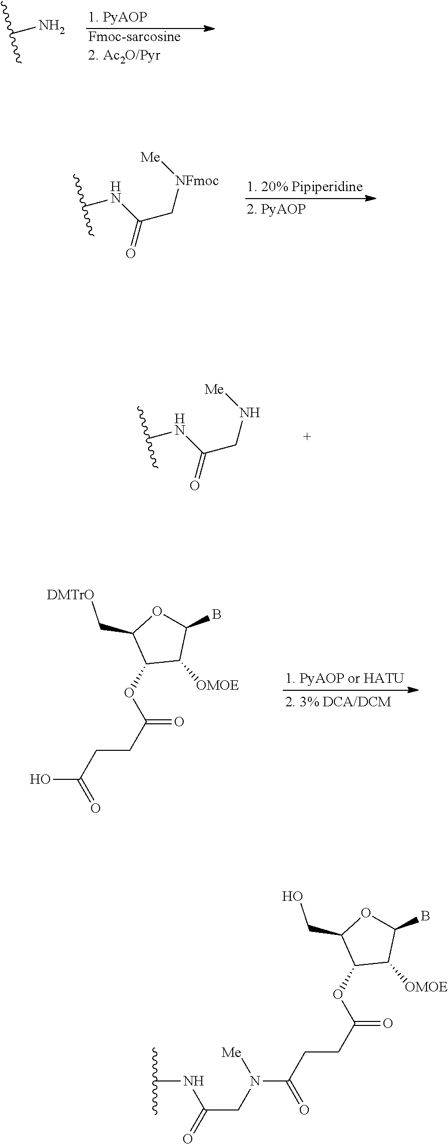

TentaGel S-NH₂ (AC354610050, ACROS Organics, loading 0.2 to 0.3 mmol/g) (4 g, ~1 mmol) was placed in a 50 mL solid phase reaction flask and washed with DMF (10 mL×3), DCM (10 mL×3) and DMF (10 mL×3). To the resin were added N-(((9H-fluoren-9-yl)methoxy)carbonyl)-N-methylglycine (3.11 g, 10.00 mmol) in DMF (5.00 mL) and ((3H-[1,2,3]triazolo[4,5-b]pyridin-3-yl)oxy)tri(pyrrolidin-1-yl)phosphonium hexafluorophosphate(V) (5.21 g, 10.00 mmol) in DMF (5 mL) followed by N-4-methylmorpholine (2199 mL, 20.00 mmol) at room temperature. It was shaken at 400 rpms. After 24 hours, the liquid was drained and the resin was rinsed with DMF (10 mL×3), DCM (10 mL×3) and DMF (10 mL×3). To the resin was added premixed pyridine (4.85 mL, 60.00 mmol) and Ac₂O (0.944 mL, 10.00 mmol) at room temperature. After 3 minutes, the solution was drained and premixed pyridine (4.85 mL, 60.00 mmol) and Ac₂O (0.944 mL, 10.00 mmol) was added at room temperature. After 3 minutes, the liquid was drained and the resin was washed with DMF (10 mL×3), DCM (10 mL×3) and DMF (10 mL×3).

Then, the resin was treated with 30 mL of 20% piperidine in DMF and the solution was collected after 3 minutes. This process was repeated 5 times and the resin was washed with DMF (10 mL×3), DCM (10 mL×3) and DMF (10 mL×3). A solution of 20% piperidine in DMF was added to the collected solution to make 300 mL in a volumetric flask. An aliquot of this solution was diluted 10-fold with 20% piperidine in DMF and the UV absorbance of the piperidine-fulvene adduct was measured (λ=301 nm, ε=7800 M⁻¹cm⁻¹, A=2.41) to give 230 μmol/g as an estimated loading.

The resin was washed with DMF (10 mL×3), DCM (10 mL×3) and DMF (10 mL×3). To the resin was added 4-(((2R,3R,4R,5R)-2-((bis(4-methoxyphenyl)(phenyl)methoxy)methyl)-4-(2-methoxyethoxy)-5-(5-methyl-4-(((E)-1-methylpyrrolidin-2-ylidene)amino)-2-oxopyrimidin-1(2H)-yl)tetrahydrofuran-3-yl)oxy)-4-oxobutanoic acid (1.5 eq., 1.198 g, 1.5 mmol)) in DMF (5 mL) followed by ((3H-[1,2,3]Triazolo[4,5-b]pyridin-3-yl)oxy)tri(pyrrolidin-1-yl)phosphonium hexafluorophosphate(V) (1.7 eq., 0.886 g, 1.7 mmol) in DMF (5 mL) and N-4-Methylmorpholine (2 eq. 0.258 g, 2 mmol). The mixture was shaken at room temperature for 3 days and washed with DMF (10 mL×3), DCM (10 mL×3) and DMF (10 mL×3).

The resin was washed with DMF (10 mL×3), DCM (10 mL×3). It was treated for 2 minutes with 3% dichloroacetic acid (DCA) in DCM (20 mL) followed by DCM (20 mL) washing to remove the DMTr group. The process was repeated (>5 times) until no color was observed. Then the resin was washed with DCM (10 mL×3), DMF (10 mL×3) and MeCN (10 mL×3).

The combined deprotection solutions were diluted with 3% DCA in DCM. The UV absorbance of the DMTr cation was measured (λ=410 nm, ε=30,400 M⁻¹cm⁻¹) to quantify the loading (0.2 mmol/g).

### Part 2. Automated Synthesis on K & A H-8-SE Oligo Synthesizer

The prepared 5′-O-DMTr-nucleotide-loaded TentaGel-SAR (20 μmol, 200 μmol/g) was packed in an empty 6 mL syringe column (Biocomma Limited, Cat #RSSC-6) and washed with MeCN. The stereopure oligonucleotides were synthesized on K &A H-8-SE Oligo Synthesizer following the cycles shown in Table 23 using stereopure PSI monomers and PO-PSI monomers. As shown in the schemes below: Sp phosphorothioate linkage was obtained using Rp-PSI-monomers that were prepared from (−)-PSI reagent; Rp phosphorothioate linkage was obtained using Sp-PSI-monomers that were synthesized from (+)-PSI, and PO internucleotide linkages were obtained using PO-PSI monomers¹.

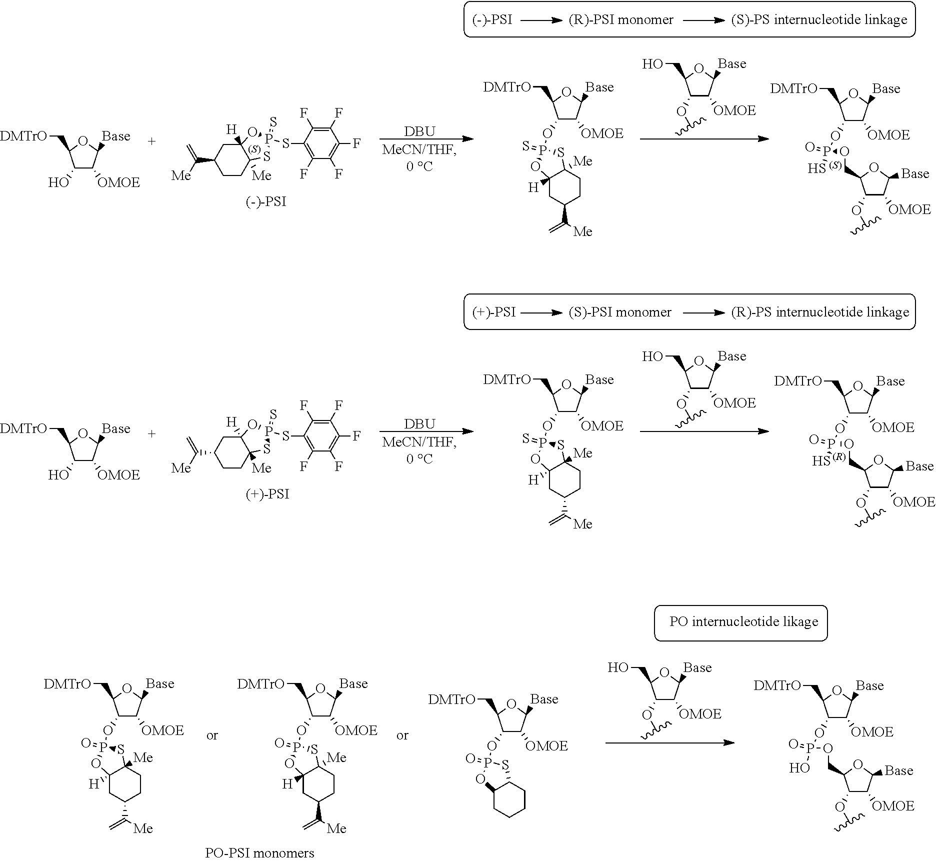

Monomers in the synthesis of Sp, Rp phosphorothioate and PO (phosphodiester) internucleotide linkages.

TABLE 23
Protocol for automated solid phase synthesis of MOE PS oligonucleotides

| | Step | Comment | Reagent/solvent | Time | | cycles |
|---|---|---|---|---|---|---|
| 1 | Deblock | PhMe wash | 3% DCA in PhMe | 1 | min | 5 |
| | | between cycles | | | | |
| 2 | Wash | | MeCN | 3 | min | 5 |
| 3 | Base Wash* | | 2,6-lutidine/DBU/MeCN (2/1/20) | 3 | min | 2 |
| 4 | Wash | | MeCN | 3 | min | 5 |
| 5 | Coupling | | Monomer (>10 eq., 0.1N), 2,6-lutidine | 16 | hr | 1 |
| | | | (>50 eq.), DBU (13 eq.) (1N/0.3N)** | | | |
| 6 | Wash | | MeCN | 3 | min | 5 |
| 7 | Capping | solvent (MeCN) | 20% AC₂O, 30% 2,6-lutidine, 20% | 1 | min | 2 |
| | | | N—Me-imidazole (1/1/1) | | | |
| 8 | Wash | | MeCN | 3 | min | 5 |

*Base wash solution: MeCN/2,6-lutidine/DBU = 20/2/1 (v/v/v)
**Coupling: monomer (0.2 mmol) in MeCN (2 mL, 0.1M) and base solution [2,6-lutidine 1.1 mL (1M), DBU 0.5 mL (0.3M), MeCN 10 mL] (0.9 mL) (>10 eq. DBU and >40 eq. 2,6-utidine) was transferred to the column. It was shaken for 16 hours, it was drained and washed, and conversion was analyzed by RP HPLC-Mass after cleavage from a bit of resin (28% NH₄OH/EtOH/NH₄OAc (9/2/1, v/v/w), 65° C., 4 hours). >95% conversion was achieved while lower conversions were observed either without 2,6-lutidine or shorter reaction time: no 2,6-lutidine, DBU (15 eq.): ~50% conversion or Monomer (10 eq.), 2,6-lutidine (50 eq.), DBU (15 eq.), 8 h: ~80% conversion.

Analytical HPLC Method 1-RP HPLC-Mass: Column: Acquity UPLC BEH C18 1.7 μm 2.1×50 mm (Part Number 186002350); Solvents: Buffer A (10 mM ammonium bicarbonate in water), Buffer B (100 mM ammonium bicarbonate/MeOH/MeCN=10/10/80); temperature: 60 °C; Flow rate: 0.8 mL/min; Gradient: 5~99% B gradient (6 min).

Part 3. Cleavage from Resin and Deprotection:

After completion of the last cycle (DMTr-On), the resin in cleavage solution (28% NH₄OH/NH₄OAc/EtOH (10/1/1, ~1 mL/1 μmol) was heated at 65 °C for 2 days in a closed bottle. It was cooled to room temperature, filtered, and then concentrated. The failed sequences were removed and DMTr group was deprotected by the below C18 cartridge protocol. The collected fractions were concentrated and purified by an Ion-Pairing Reverse-Phase (IR-RP) HPLC.

### C18 Column Protocol:

Sep-Pak cartridge [Waters, Sep-Pak Vac 35 cc (10 g) C18 Cartridge] was equilibrated with MeOH (2 CV), MeCN (2 CV) followed by 2 N Et₃NHOAc (2 column volumes (CV)). The crude sample in 0.1 N Et₃NHOAc was loaded on a cartridge. The cartridge was washed with 2 N NaCl/MeCN (5/1, v/v) to elute truncated sequences, and 3% TFA in water (150 mL), then water (50 mL). The crude DMTr-off PS-oligonucleotide was eluted with 50 mL of acetonitrile-water (1:1, v/v) containing 0.5% of 28% NH₄OH. The solution containing crude DMTr-off oligonucleotide was dried under vacuum. The weight was measured by Nanodrop (RNA-40) and ³¹P NMR was taken. It was analyzed by RP-HPLC, IEX-HPLC and UPLC/MS.

Analytical HPLC Method 2-Ion-pairing RP HPLC-Mass: Column: XBridge Premier BEH C18 (2.5 μm, 150×2.1 mm); Temperature: 60 °C; Flow rate: 1 mL/minute; Detection wavelength: 260 nm; Solvents: buffer A: 100 mM HFIP/8.6 mM Et₃N (H₂O), buffer B:100% MeOH; Gradient: 5% to 30% B gradient (15 minutes).

Analytical HPLC Method 3-Ion-pairing RP HPLC-Mass: Column: XBridge Premier BEH C18 (300A, 2.5 μm, 150×2.1 mm); Temperature: 60 °C, Flow rate: 0.5 mL/minute; Detection wavelength: 260 nm; Solvents: Buffer A: 100 mM n-C₆H₁₃NH₃OAc (H₂O/MeCN 9/1) Buffer B: 100 mM C₆H₁₃NH₃OAc (H₂O/MeCN 1/1); Gradient: 80% to 100% B gradient (15 minutes).

Analytical HPLC Method 4-Ion-pairing RP HPLC-Mass: Column: XBridge Premier BEH C18 (300 Å, 2.5 μm, 150×2.1 mm); Temperature: 60 °C, Flow rate: 0.5 mL/min. Detection wavelength: 260 nm; Solvents: buffer A: 10 mM n-Hexylamine/50 mM HFIP in water, buffer B: MeCN; Gradient: 23~28% Buffer B gradient (15 minutes).

### Part 4. HPLC Purification and Desalting:

The crude material after SepPak treatment was purified by a ion-pairing RP HPLC by the following methods using sterile water (WFI from Baxter, VWR cat. 68000-955).

Preparative HPLC Method 1: Column: XBridge Prep C18 OBD Prep (10 μm, 19×250 mm); Flow rate: 30 mL/minute. Detection wavelength: 260 nm; Solvents: buffer A: 8.6 mM TEA/100 mM HFIP in water, Buffer B: MeOH; Gradient: 10-37% Buffer B gradient (30 minute).

Preparative HPLC Method 2: Column: Xbridge BEH C18 (10 μm, 10×250 mm); Flow rate: 14 mL/minute. Detection wavelength: 260 nm; Solvents: buffer A: 100 mM C₆H₁₃NH₃OAc (H₂O/MeCN 9/1), Buffer B: 100 mM C₆H₁₃NH₃OAc (H₂O/MeCN 1/1); Gradient: 50% to 75% gradient (26 min)

Preparative HPLC Method 3: Column: XBridge C18 OBD Prep (300 Δ, 5 μm, 19×250 mm); Flow rate: 30 mL/minute; Detection wavelength: 260 nm; Solvents: buffer A: 10 mM HA/50 mM HFIP in water, Buffer B: MeCN; Gradient: 23~28% Buffer B gradient (30 minutes).

The fractions containing the desired compound were concentrated and dissolved with 0.2 N NaCl in EtOH/water (1/4). The resulting solution was desalted by membrane filtration by using a 3000 MW cut-off (3K centrifugal membrane tube, Amicon Ultra-15, Ultracel-3K (3400 rpm, 45 minutes) (cat.UFC900396 from Sigma-Aldrich) or Macrosep Devices (cat. MAP003C38) from PALL, 3400 rpm, 40 minutes, 15 mL WFI×3). The final desalted solution was filtered (0.2 micron sterile syringe filter). The absorbance of the diluted solution was measured at 260 nm on a Nanodrop UV-Vis spectrophotometer to give a yield (7~15% yield) and endotoxin level was confirmed to be less than 0.06 EU/mg by a kinetic chromogenic LAL method (Charles River, Endosafe® nexgen-PTS).

Part 5. Tm Measurement with Reverse Complementary RNA and NMR

### Tm Measurement Device: Shimadzu UV-2700 UV-Vis Spectrophotometer

Protocol 1: ASO samples were prepared at a concentration of 400 μM using deionized water. IDT's reverse complementary RNA (rcRNA) was dissolved to 400 μM using UltraPure Distilled water. 10 μL aliquots of each stock solutions were diluted to 1 mL using ultra pure distilled water and their actual concentrations were measured by UV-Vis Spectrophotomer. Test samples (500 μL) were prepared containing 4.0 μM ASO with 4.0 μM rcRNA in buffer (100 mM NaCl, 10 mM Na phosphate pH 7.0 with 0.1 mM EDTA). Test samples were incubated in a 1 mL cuvette and heated from 15 °C to 105 °C at 0.5 °C/minute. UV absorbance increase due to strand melting was monitored at 260 nm. Prior to the experiment, the samples were melted and reannealed by heating from 25 °C to 95 °C at 5 °C/minute and cooling to starting temperatures to ensure complete annealing. Shimadzu Tm Analysis software was used to calculate the Tm (curve inflection point: 50% melting) using the derivative function.

Protocol 2: ASO samples were prepared at a concentration of 200 μM using PBS and then followed the same procedure as protocol 1 with adjusted amount.

³¹P NMR (162 MHz)³ was taken in stock phosphate buffer (100 mM, pD=7.4) that was prepared with 135.5 mg of K₂DPO₄ and 31.2 mg of KD₂PO₄ in 10 mL D₂O after C18 purification and deprotection of DMTr. See Evstigneev et al., “Hexamer oligonucleotide topology and assembly under solution phase NMR and theoretical modeling scrutiny,” Biopolymers 2010, 93 (12), 1023-1038.

### Exemplary Compounds

All nucleotides are 2′-MOE unless specified and “C” represent 5′-Methyl cytosine.

### A. Compound MOE-277: 20Mer, all Sp

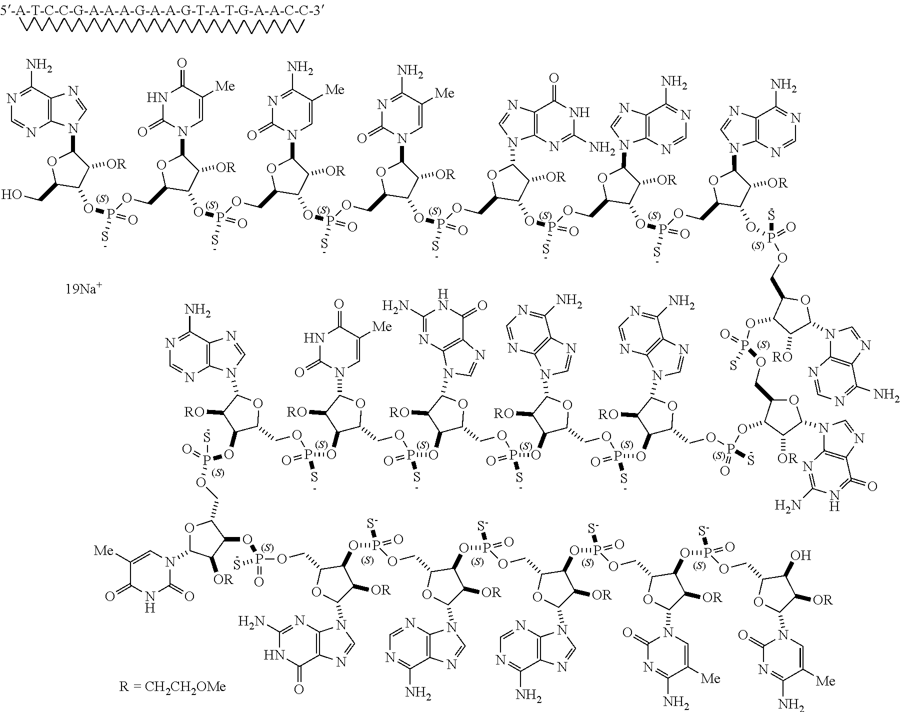

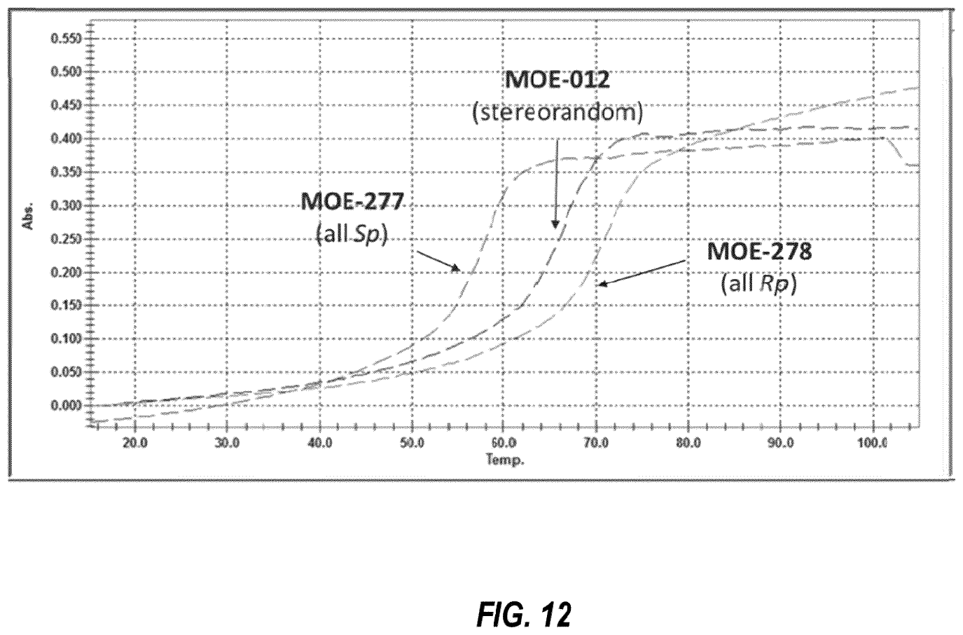

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.05; Tm=57.8 °C (Tm of stereorandom=66.5 °C) by Protocol 1. The Tm of MOE-277 is shown in FIG. 12.

### B. Compound MOE-278: 20Mer, all Rp

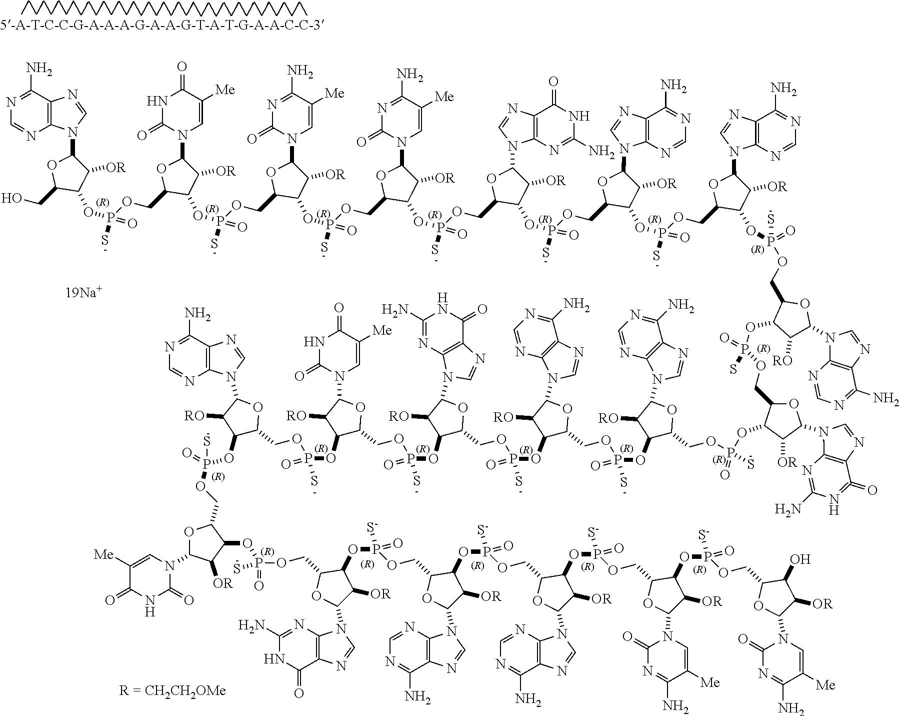

Purified by Preparative HPLC Method 2: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.492; Tm=71.5 °C (Tm of stereorandom=66.5 °C) by Protocol 1. The Tm of MOE-278 is shown in FIG. 12.

C. Compound MOE-279: 20mer, 4Rp

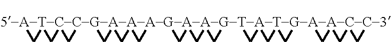

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.25. Tm=61.4 °C (Tm of stereorandom=66.5 °C) by Protocol 1.

D. Compound MOE-280: 20mer, 5Rp

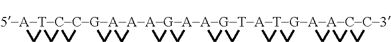

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1994.88. Tm=62.7 °C (Tm of stereorandom=66.5 °C) by Protocol 1.

E. Compound MOE-281: 20mer, 7Rp

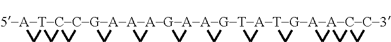

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.06. Tm=62.3 °C (Tm of stereorandom=66.5 °C) by Protocol 1.

F. Compound MOE-282: 20mer, 7Rp

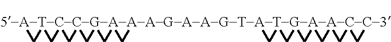

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1994.81. Tm=63.5 °C (Tm of stereorandom=66.5 °C) by Protocol 1.

G. Compound MOE-283: 20mer, 9Rp

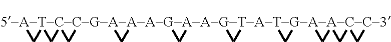

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.43. Tm=64.8 °C (Tm of stereorandom=66.5 °C) by Protocol 1.

H. Compound MOE-284: 20mer, 10Rp

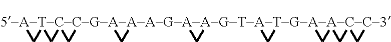

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.50; Tm=63.4 °C (Tm of stereorandom=66.5 °C) by Protocol 1.

I. Compound MOE-285: 20mer, 9Rp

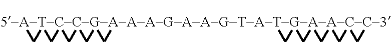

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.50; Tm=63.4 °C (Tm of stereorandom=66.5 °C) by Protocol 1.

J. Compound MOE-286: 20mer, 13Rp

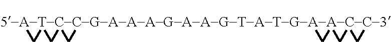

Purified by Preparative HPLC Method 2: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1994.55.

K. Compound MOE-287: 20mer, 3Rp

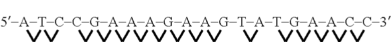

Purified by Preparative HPLC Method 2: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.08; Tm=59.7 °C (Tm of stereorandom=66.5 °C) by Protocol 1.

FIG. 12 shows the Tms of MOE-012, MOE-277, and MOE-278.

L. Compound MOE-288: 18mer, all Sp

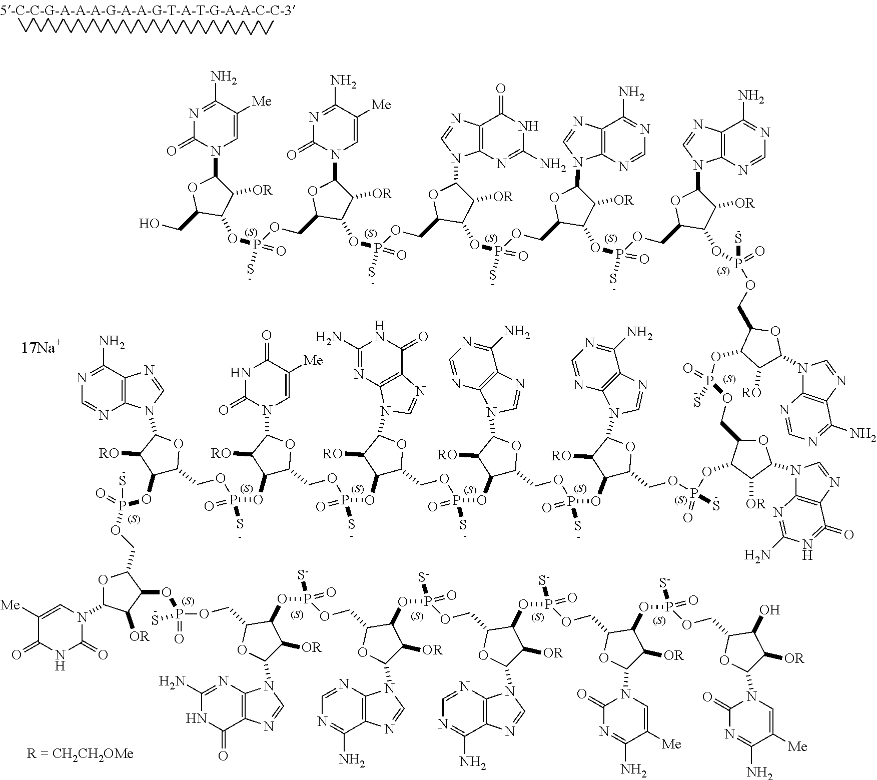

Purified by Preparative HPLC Method 3: C₂₃₄H₃₅₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.45; Tm=58.4 °C (Tm of stereorandom=65.9 °C) by Protocol 2.

³¹P NMR (162 MHz) δ ppm 56.09, 55.82, 55.78, 55.56, 55.52, 55.32, 55.21, 55.15, 55.06

M. Compound MOE-289: 18mer, all Rp

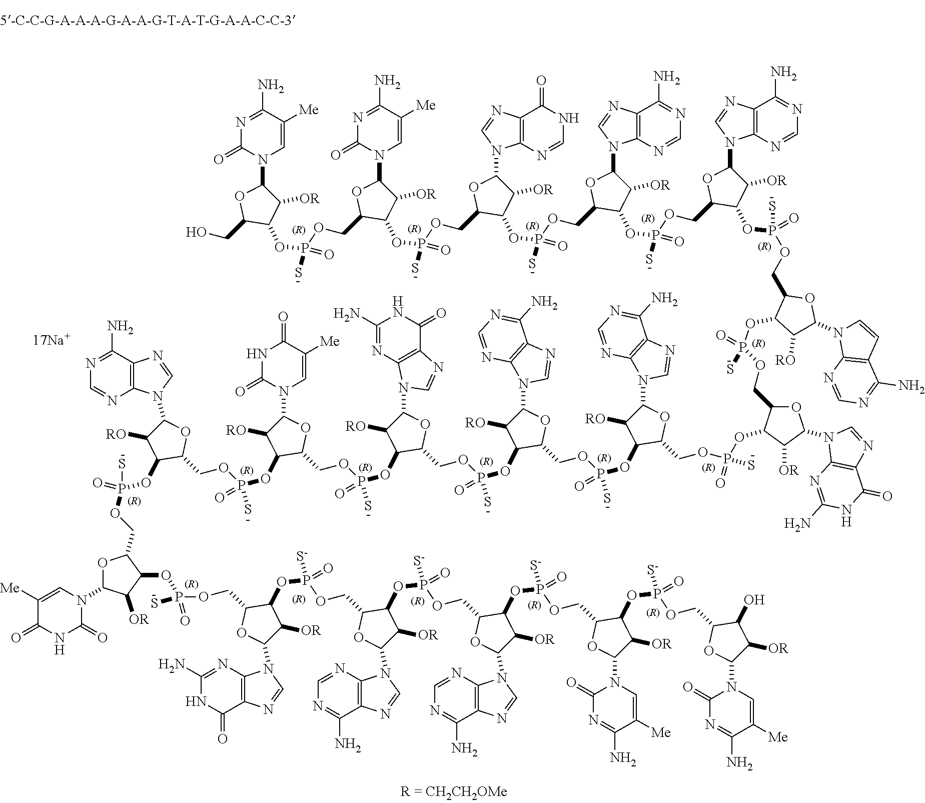

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.38; Tm=70.4 °C (Tm of stereorandom=65.9 °C) by Protocol 2.

³¹P NMR (162 MHz) δ ppm 56.09, 55.82, 55.78, 55.56, 55.52, 55.32, 55.21, 55.15, 55.06

N. Compound MOE-290: 18mer, 11Rp

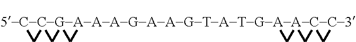

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.57; Tm=66.6 °C (Tm of stereorandom=65.9 °C) by Protocol 2.

³¹P NMR (162 MHz) δ ppm 58.53, 58.22, 58.08, 57.82, 57.60, 57.40, 57.12, 55.67, 55.54, 55.30, 55.12

O. Compound MOE-291: 18mer, 8Rp

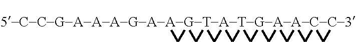

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.31; Tm=62.5 °C (Tm of stereorandom=65.9 °C) by Protocol 2.

³¹P NMR (162 MHz) δ ppm 57.07, 56.83, 56.71, 56.54, 56.31, 55.16, 54.81, 54.33, 54.24, 54.12, 54.23

P. Compound MOE-292: 18mer, 8Rp

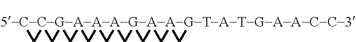

Purified by Preparative HPLC Method 3: C₂₄₃H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1794.95; Tm=62.6 °C (Tm of stereorandom=65.9 °C) by Protocol 2.

³¹P NMR (162 MHz) δ ppm 60.01, 59.40, 59.36, 58.87, 58.50, 58.14, 57.67, 57.37, 57.15, 56.66, 56.48, 55.83, 55.55, 55.26

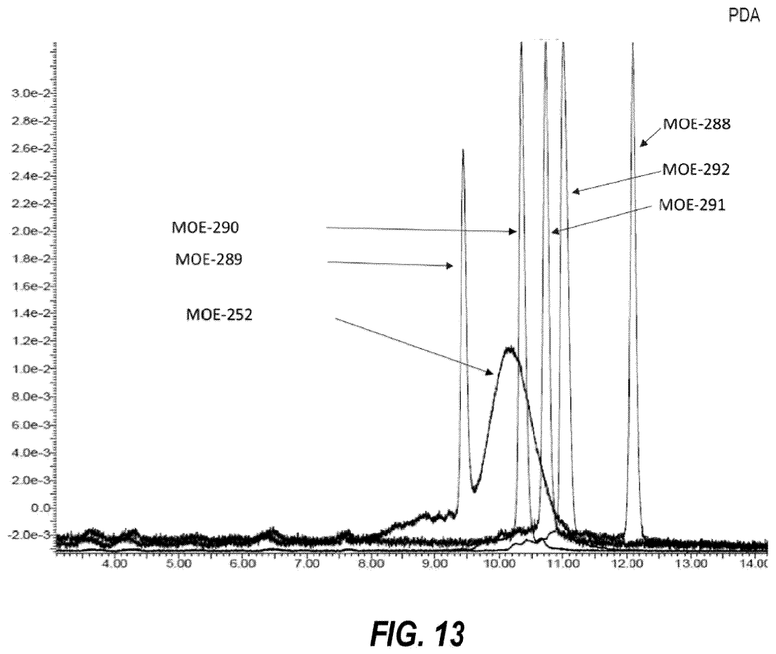

FIG. 13 shows an example of overlay HPLC chromatogram (MOE-252 and MOE-288 to MOE-292 by Analytical HPLC Method 4.

Q. Compound MOE-293: 18mer, 4Rp

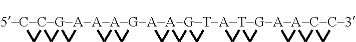

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.95; Tm=59.5 °C (Tm of stereorandom=65.9 °C) by Protocol 2.

³¹P NMR (162 MHz) δ ppm 56.79, 56.19, 55.09, 54.93, 54.85, 54.67, 54.53

R. Compound MOE-294: 18mer, 6Rp

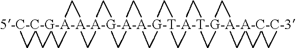

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.54; Tm=59.7 °C (Tm of stereorandom=65.9 °C) by Protocol 2.

³¹P NMR (162 MHz) δ ppm 57.84, 57.43, 57.17, 56.92, 56.80, 55.98, 55.86, 55.62, 55.58, 55.46, 55.27, 55.11, 55.06, 55.00

S. Compound MOE-295: 18mer, 4Rp

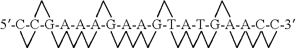

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.82; Tm=60.6 °C (Tm of stereorandom=65.9 °C) by Protocol 2.

³¹P NMR (162 MHz) δ ppm 57.71, 57.25, 57.06, 56.11, 55.79, 55.68, 55.48, 55.35, 55.21, 55.11

T. Compound MOE-296: 18mer, 2Rp/2PO

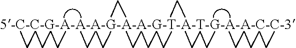

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.56; Tm=61.3 °C (Tm of stereorandom=65.9 °C) by Protocol 2.

³¹P NMR (162 MHz) δ ppm 58.26, 58.20, 57.84, 57.65, 57.49, 57.40, 57.16, 56.97, 0.33

U. Compound MOE-297: 18mer, 4Rp/2PO

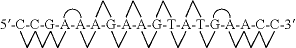

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.35; Tm=62.7 °C (Tm of stereorandom=65.9 °C) by Protocol 2.

³¹P NMR (162 MHz) δ ppm 57.24, 57.04, 56.42, 56.36, 55.83, 55.69, 55.56, 55.36, 55.17, 55.07, 54.64, −1.03, −1.13

V. Compound MOE-298: 18mer, 2Rp/2PO

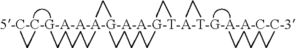

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.40; Tm=61.6 °C (Tm of stereorandom=65.9 °C) by Protocol 2.

³¹P NMR (162 MHz) δ ppm 57.20, 57.08, 56.77, 56.55, 56.17, 56.10, −0.72

W. Compound MOE-299: 20mer, 2R/2PO

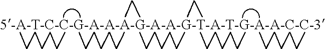

Purified by Preparative HPLC Method 3: C₂₆₀H₃₇₂N₈₃O₁₃₅P₁₉S₁₇ Mw=7950.51 with a theoretical value of m/z 1986.62 as the [M-4H]⁴⁻ ion using the most abundant natural isotopes, was detected by low resolution mass spectrometry at m/z 1987.01; Tm=61.5 °C (Tm of stereorandom=69.6 °C) by Protocol 1.

³¹P NMR (162 MHz) δ ppm 57.48, 57.22, 56.14, 55.90, 55.65, 55.77, 55.38, 55.30, 55.25, 55.21, 55.06, 54.94, −0.95, −0.99

X. Compound MOE-300: 18mer, 6Rp/2PO

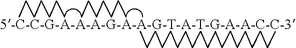

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.49; Tm=63.2 °C (Tm of stereorandom=65.9 °C) by Protocol 2.

³¹P NMR (162 MHz) δ ppm 58.39, 58.07, 57.85, 57.69, 56.36, 56.06, 55.78, 55.70, 55.57, 55.38, 55.33, 55.29, −0.97

Y. Compound MOE-301: 18mer, 5Rp/2PO

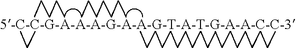

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.66; Tm=62.2 °C (Tm of stereorandom=65.9 °C) by Protocol 2.

³¹P NMR (162 MHz) δ ppm 58.57, 58.02, 57.81, 57.65, 56.31, 56.02, 55.73, 55.64, 55.52, 55.33, 55.27, 55.25, 55.11, −1.00

Z. Compound MOE-303: 18mer, 3PO

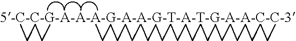

Purified by Preparative HPLC Method 3: C₂₃₄H₃₃₅N₇₆O₁₂₂P₁₇S₁₄ Mw=7137.40 with a theoretical value of m/z 1783.25 as the [M-4H]⁴⁻ ion using the most abundant natural isotopes, was detected by low resolution mass spectrometry at m/z 1782.76; Tm=59.5 °C (Tm of stereorandom=65.9 °C) by Protocol 2.

³¹P NMR (162 MHz) δ ppm 56.36, 56.11, 55.93, 55.84, 55.69, 55.64, 55.53, 55.38, −0.88, −0.97

AA. Compound MOE-304: 18mer, 5PO

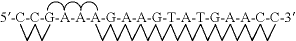

Purified by Preparative HPLC Method 3: C₂₃₄H₃₃₅N₇₆O₁₂₄P₁₇S₁₂ Mw=7106.45 with a theoretical value of m/z 1775.61 as the [M-4H]⁴⁻ ion using the most abundant natural isotopes, was detected by low resolution mass spectrometry at m/z 1775.83; Tm=61.6 °C (Tm of stereorandom=65.9 °C) by Protocol 2.

³¹P NMR (162 MHz, Solvent) δ ppm 56.29, 55.75, 55.71, 55.65, 55.53, 55.42, −0.62, −0.79, −0.89, −0.98

BB. Compound MOE-305: 18mer, 4PO

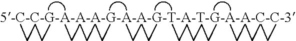

Purified by Preparative HPLC Method 3: C₂₃₄H₃₅₅N₇₆O₁₂₃P₁₇S₁₃ Mw=7122.43 with a theoretical value of m/z 1779.50 as the [M-4H]⁴⁻ ion using the most abundant natural isotopes, was detected by low resolution mass spectrometry at m/z 1779.42; Tm=61.4 °C (Tm of stereorandom=65.9 °C) by Protocol 2.

³¹P NMR (162 MHz) δ ppm 55.54, 55.49, 55.72, 55.26, 55.14, 55.11, 54.98, −1.02, −1.06, −1.14, −1.47

### CC. Compound MOE-306: 18mer 3PO

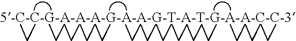

Purified by Preparative HPLC Method 3: C₂₃₄H₃₃₅N₇₆O₁₂₂P₁₇S₁₄ Mw=7137.40 with a theoretical value of m/z 1783.25 as the [M-4H]⁴⁻ ion using the most abundant natural isotopes, was detected by low resolution mass spectrometry at m/z 1783.54; Tm=60.3 °C (Tm of stereorandom=65.9 °C) by Protocol 2.

³¹P NMR (162 MHz) δ ppm 56.18, 55.88, 55.53, 55.37, 55.30, 55.64, 55.16, 55.07, −0.84, −0.90, −0.95

DD. Compound MOE-307: 18mer, 2PO

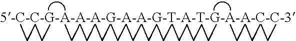

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1788.19; Tm=59.0 °C (Tm of stereorandom=65.9 °C) by Protocol 2.

³¹P NMR (162 MHz) δ ppm 56.03, 55.84, 55.70, 55.56, 55.45, 55.27, 55.24, 55.11, 54.95, −1.10, −1.20

EE. Compound MOE-308: 20mer, 2PO

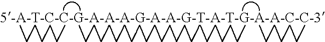

Purified by Preparative HPLC Method 3: C₂₆₀H₃₇₂N₈₃O₁₃₅P₁₉S₁₇ Mw=7950.51 with a theoretical value of m/z 1986.62 as the [M-4H]⁴⁻ ion using the most abundant natural isotopes, was detected by low resolution mass spectrometry at m/z 1986.92; Tm=58.5 °C (Tm of stereorandom=69.6 °C) by Protocol 1.

³¹P NMR (162 MHz) δ ppm 55.91, 55.76, 55.52, 55.20, 55.10, 54.99, 54.89, −0.99, −1.05, −1.09

FF. Compound MOE-309: 20mer, 4PO

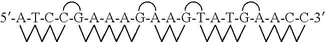

Purified by Preparative HPLC Method 3: C₂₆₀H₃₇₂N₈₃O₁₃₇P₁₉S₁₅ Mw=7919.50 with a theoretical value of m/z 1978.87 as the [M-4H]⁴⁻ ion using the most abundant natural isotopes, was detected by low resolution mass spectrometry at m/z 1978.66; Tm=62.8 °C (Tm of stereorandom=69.6 °C) by Protocol 1.

³¹P NMR (162 MHz) δ ppm 55.83, 55.65, 55.50, 55.41, 55.19, 55.02, 55.11, 54.77, −1.04, −1.11, −1.15, −1.55

GG. Compound MOE-310: 18mer, 5R/2PO

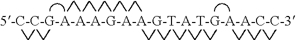

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.64; Tm=63.4 °C (Tm of stereorandom=65.9 °C) by Protocol 2.

³¹P NMR (162 MHz) δ ppm 58.22, 57.54, 56.08, 55.88, 55.41, 55.26, 55.17, 55.10, 55.04, 54.95, −1.02

HH. Compound MOE-311: 18mer, 4R/2PO

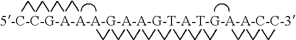

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.34; Tm=61.0 °C (Tm of stereorandom=65.9 °C) by Protocol 2.

³¹P NMR (162 MHz) δ ppm 58.21, 57.66, 56.05, 55.89, 55.57, 55.48, 55.38, 55.28, 55.26, 55.05, 54.96, −1.03, −1.25

### Example 10: Structures of PMO-002 and PMO-424

### PMO-002

Stereo-random PMO-002 oligonucleotide sequence was synthesized according to the methods disclosed above in Example 2.

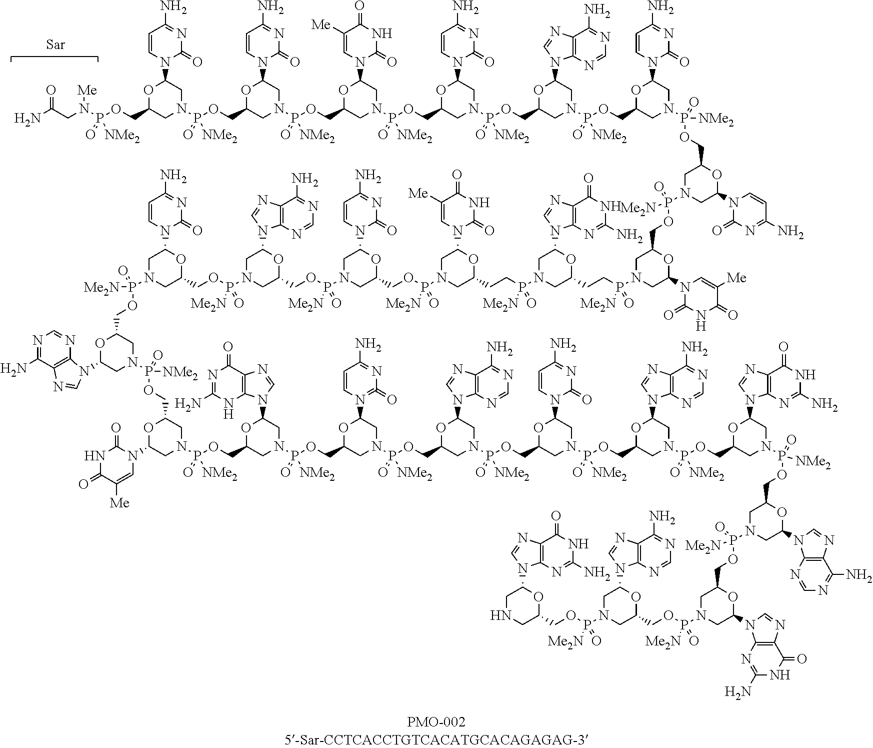

### PMO-424

Stereopure PMO-424 was synthesized according to the methods disclosed above in Example 5. The structure of PMO-424 with a full Sp phosphorodiamidate backbone is shown here:

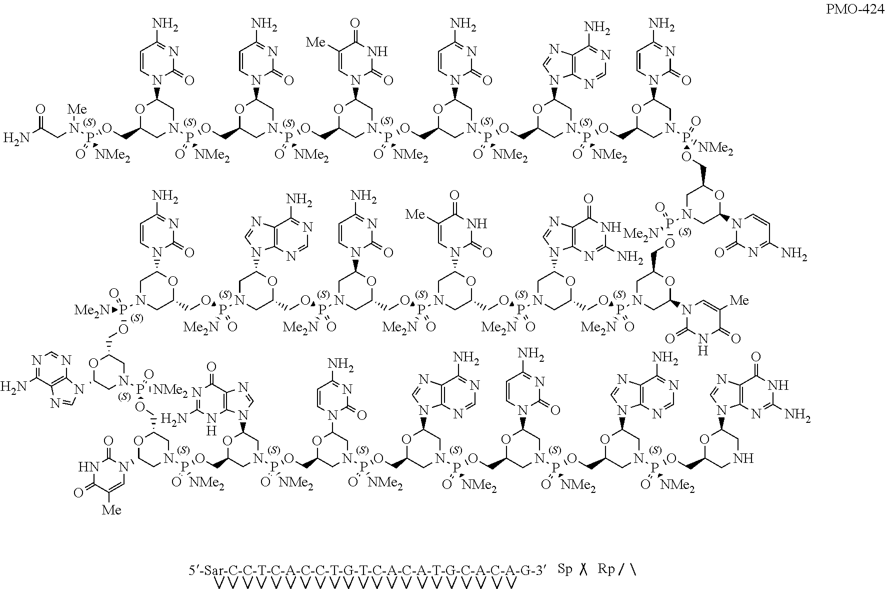

P³¹ NMR (D₂O, 162 MHz) δ 21.5, 18.7, 18.6, 18.5, 18.4, 18.3, 18.3, 18.1, 18.0, 17.9.

ESI-TOF-MS Calcd.: 7009.02 for C₂₄₆H₃₉₀N₁₁₉O₈₄P₂₁.

Found: 7008.51.

### Example 11: DBCO-Functionalized PMO-002 (Compound 9)

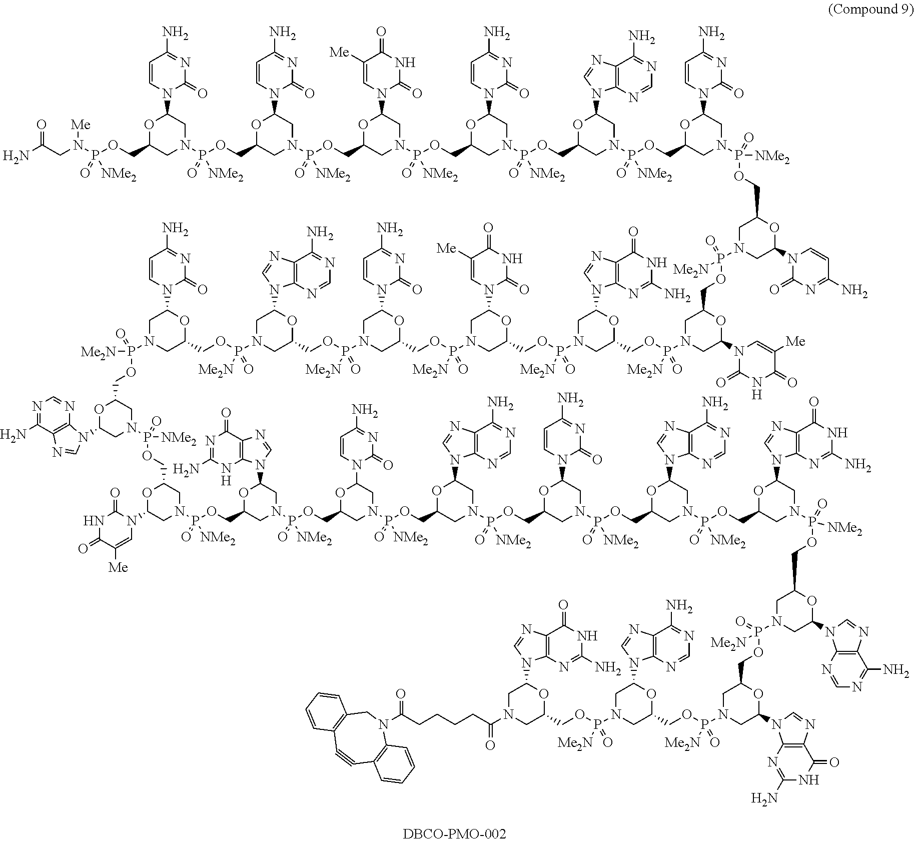

PMO-002 (40 mg) and DMSO (500 uL) were added to a vial and the mixture was warmed to 39 °C until a clear solution was obtained, then cooled to room temperature. DBCO-C6 acid (CAS 1425485-72-8) (4.8 mg, 3.0 equiv.) and Hunig's base (4.2 μL, 5 equiv.) in NMP (500 μL) were added to the PMO solution. HOBt (2.2 mg, 3 equiv.) was added to this mixture, followed by HBTU (5.4 mg, 3 equiv.) in NMP (50 μL) added via syringe. The reaction mixture was immediately heated to 40 °C for three hours. Upon completed conversion by HPLC, the reaction mixture was cooled to room temperature, then aqueous NH₄OH (28%, 200 μL) was added and stirred for two hours. The solvent was partially evaporated under nitrogen stream, then MeCN (3 mL) and EtOAc (3 mL) were added. The white precipitate was packed by centrifugation and the supernatant was decanted. The residue was dissolved in water (3 mL) and filtered over a syringe filter to remove particulates. Purification by reverse phase chromatography using the method below in Table 24 afforded Compound 9 DBCO-PMO-002 (13 mg).

LRMS: Calcd. m/z for [M+6H]⁶⁺ ion of C₃₁₅H₄₇₉N₁₄₈O₁₀₀P₂₅ (8709.04): 1452.51. found: 1452.81.

TABLE 24
Purification method for PMO-alkyne conjugates.

| | |
|---|---|
| Analytical | |
| Column | XBridge, Premier BEH C18 300 Å 2.5 μm 150 × 2.1 mm |
| | (Part number: 186009894) |
| Flow rate | 0.8 mL/min |
| Column temperature | 60 °C |
| Solution A | 10 mM ammonium bicarbonate in water |
| Solution B | 10 mM ammonium bicarbonate/MeOH/MeCN |
| | (10/10/80) |
| Gradient | 5 to 99% B (6 min) |
| Preparative | |
| Column | Waters, XBridge Prep C18 OBD 5 μm, 19 × 100 mm |
| | (Part number: 186002978) |
| Flow rate | 25.5 mL/min |
| Column temperature | room temperature |
| Solution A | 10 mM ammonium bicarbonate in water |
| Solution B | 10 mM ammonium bicarbonate/MeOH/MeCN |
| | (10/10/80) |
| Gradient | 5 to 28% B (28 min) |

### Example 12: BCN-Functionalized-PMO-002 (Compound 10)

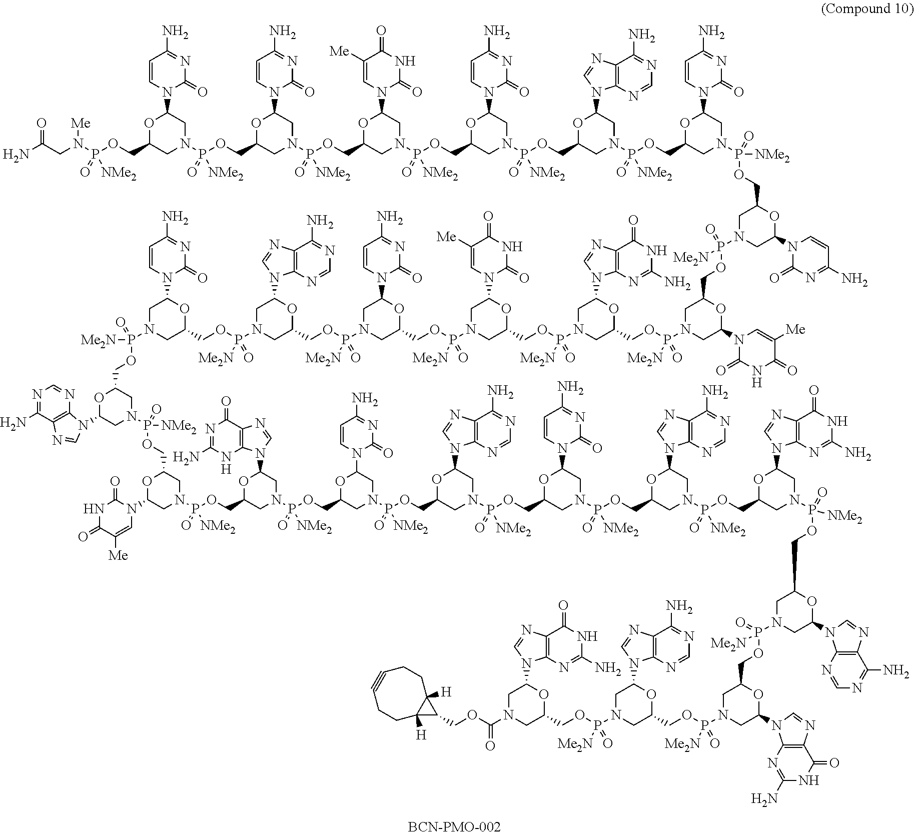

PMO-002 (25 mg, 2.98 μmol), aqueous sodium tetraborate (0.1 M, 300 μL) and 30 μL aq NaHCO3 (saturated aqueous) were added to a flask. Upon dissolution of the PMO-002, ((1R,8S,9s)-bicyclo[6.1.0]non-4-yn-9-yl)methyl (2,5-dioxopyrrolidin-1-yl) carbonate (2.60 mg, 8.93 μmol) dissolved in DMSO (250 μL) was added to the flask. The reaction mixture was stirred overnight and then MeCN (10 mL) was added. The precipitate was packed via centrifuge and then the solvent decanted. The solid Compound 10 was washed with MeCN, dried, then used in the click reaction described below in Example 15 as is (without additional purification).

LRMS: Calcd. m/z for [M+6H]⁶⁺ ion of C₃₀₅H₄₇₄N₁₄₇O₁₀₀P₂₅ (8570.0): 1429.33. found: 1429.58.

### Example 13: Peptides for Conjugation with ASOs

Peptides were synthesized by standard Fmoc solid-phase synthesis protocols. R. Behrendt et al., 22 J. PEPT. Sci. 4-27 (2016). Structures and low resolution (LR) MS data for peptide is included where applicable. B is beta alanine; X is 6-aminohexanoic acid; other standard single-letter amino acid abbreviations are used.

### Compound 11

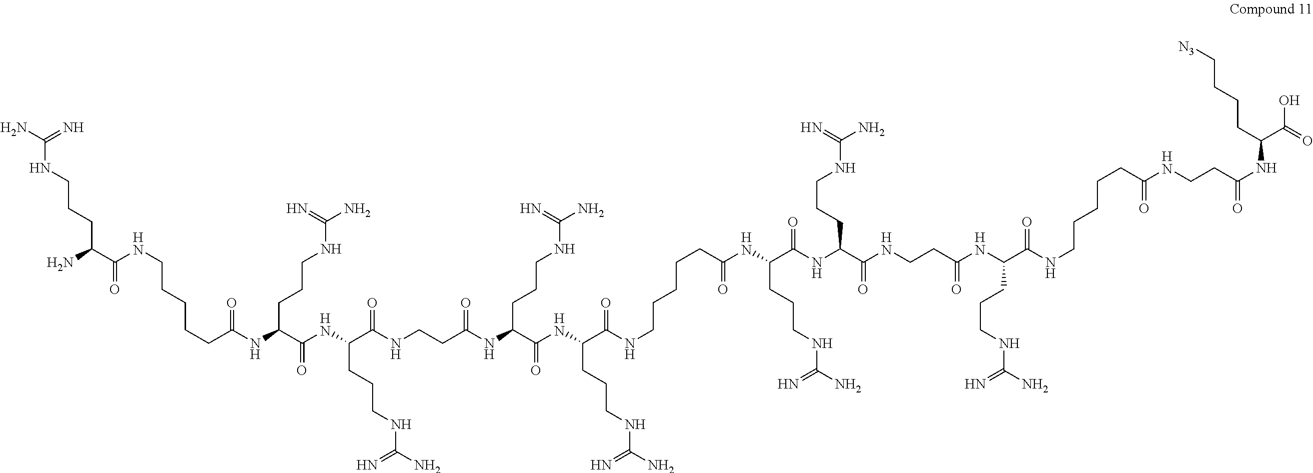 [SMILES: [N-]=[N+]=NCCCC[C@H](NC(=O)CCNC(=O)CCCCCNC(=O)[C@H](CCCNC(=N)N)NC(=O)CCNC(=O)[C@H](CCCNC(=N)N)NC(=O)[C@H](CCCNC(=N)N)NC(=O)CCCCCNC(=O)[C@H](CCCNC(=N)N)NC(=O)[C@H](CCCNC(=N)N)NC(=O)CCNC(=O)[C@H](CCCNC(=N)N)NC(=O)[C@H](CCCNC(=N)N)NC(=O)CCCCCNC(=O)[C@@H](N)CCCNC(=N)N)C(=O)O]

Compound 11: RXRRBRRXRRBRXB-azidolysine; MW=1974.42, LRMS: Calcd. m/z for [M+5H]⁵⁺ ion of C₈₁H₁₅₆N₄₂O₁₆ (1974.27): 395.85. found: 395.8.

### Compounds 12 and 13

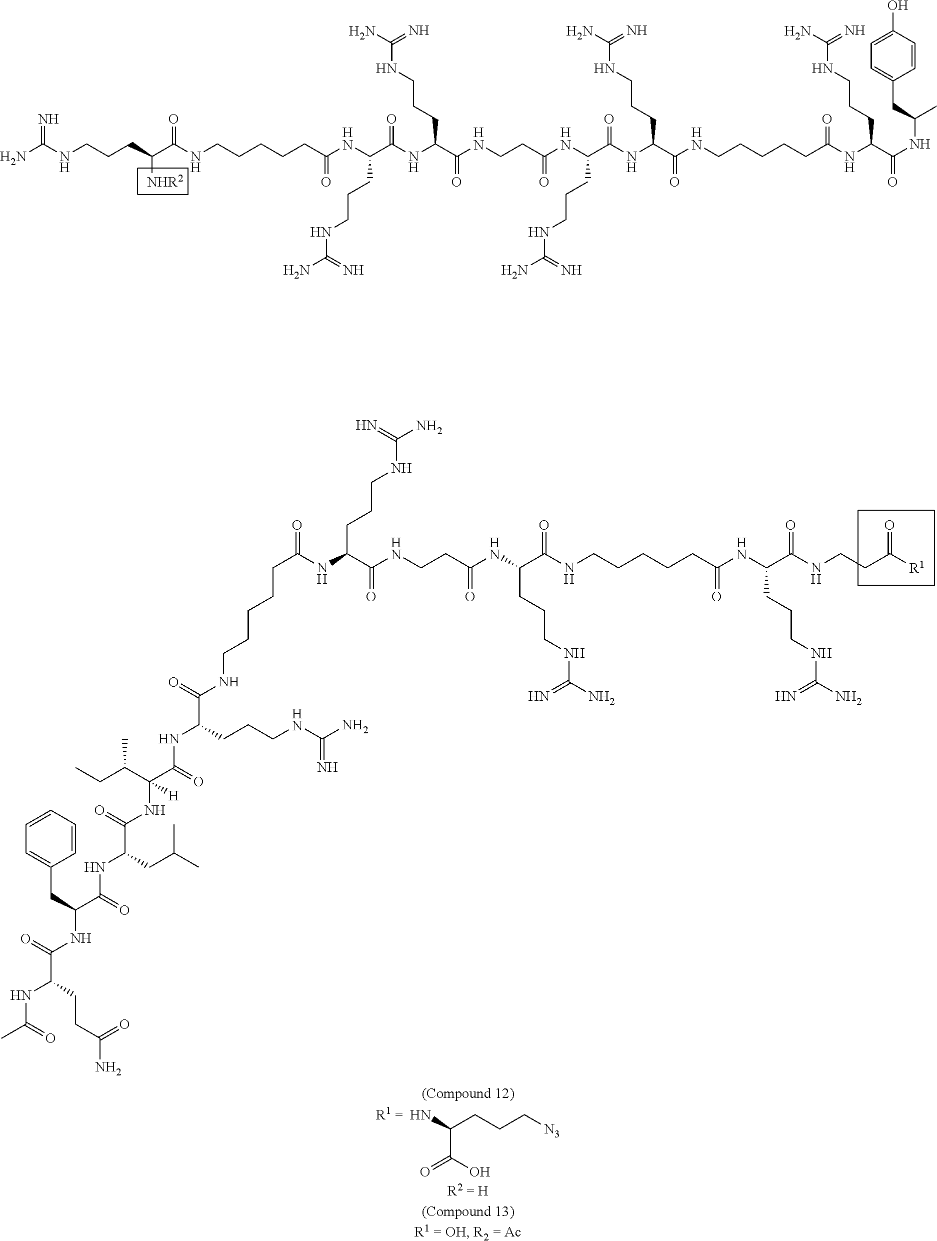

For a click reaction, R¹ and R² are the options shown for (Compound 12) in the box above: RXRRBRRXRYQFLIRXRBRXRB-azidolysine; (MW=3064.7, LRMS: Calcd. m/z for [M+7H]7+ ion of C134H239N57O26 (3063.92): 438.7. found: 438.8.) was used. For amide coupling, R¹ and R² are the options shown for (Compound 13) in the box above: Ac-RXRRBRRXRYQFLIRXRBRXRB-OH.

### Compound 14

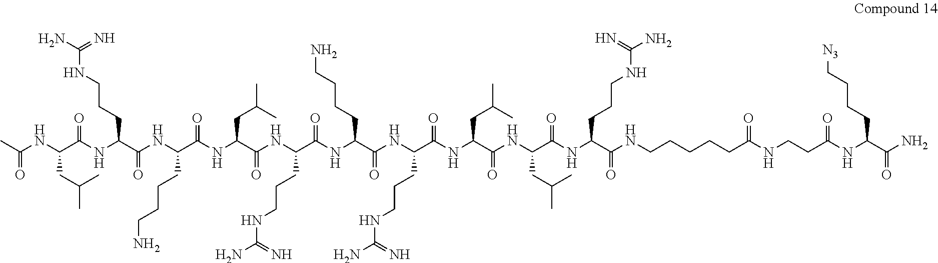 [SMILES: CC(=O)N[C@@H](CC(C)C)C(=O)N[C@@H](CCCNC(=N)N)C(=O)N[C@@H](CCCCN)C(=O)N[C@@H](CC(C)C)C(=O)N[C@@H](CCCNC(=N)N)C(=O)N[C@@H](CCCCN)C(=O)N[C@@H](CCCNC(=N)N)C(=O)N[C@@H](CC(C)C)C(=O)N[C@@H](CC(C)C)C(=O)N[C@@H](CCCNC(=N)N)C(=O)NCCCCCC(=O)NCCC(=O)N[C@@H](CCCCN=[N+]=[N-])C(N)=O]

Compound 14: Ac-LRKLRKRLLRXB-azidolysine: MW=1731.23, LRMS: Calcd. m/z for [M+2H]²⁺ ion of C₇₇H₁₄₇N₃₁O₁₄ (1731.17): 866.58. found: 866.4.

### Compound 15

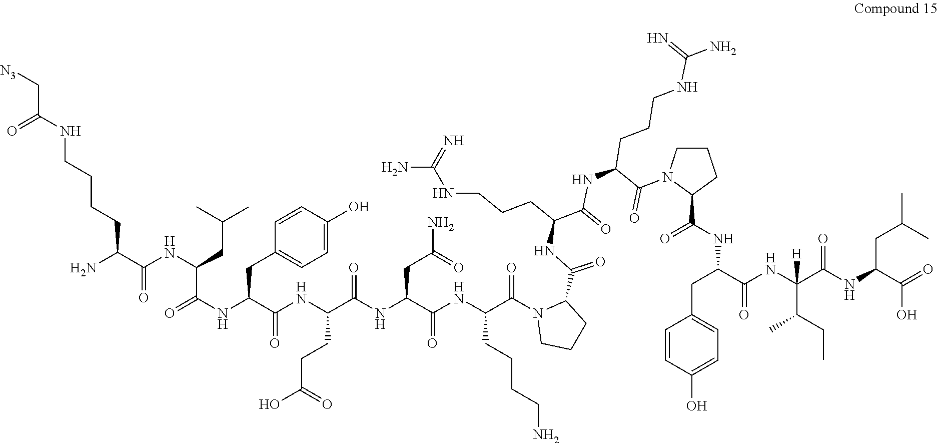 [SMILES: CC[C@H](C)[C@H](NC(=O)[C@H](Cc1ccc(O)cc1)NC(=O)[C@@H]1CCCN1C(=O)[C@H](CCCNC(=N)N)NC(=O)[C@H](CCCNC(=N)N)NC(=O)[C@@H]1CCCN1C(=O)[C@H](CCCCN)NC(=O)[C@H](CC(N)=O)NC(=O)[C@H](CCC(=O)O)NC(=O)[C@H](Cc1ccc(O)cc1)NC(=O)[C@H](CC(C)C)NC(=O)[C@@H](N)CCCCNC(=O)CN=[N+]=[N-])C(=O)N[C@@H](CC(C)C)C(=O)O]

Compound 15: K(azidoacetyl)-LYENKPRRPYIL: MW=2520.89, LRMS: Calcd. m/z for [M+4H]⁴⁺ ion of C₁₁₄H₁₇₈N₃₄O₃₁ (2519.34): 630.8. found: 631.2.

### Compound 16

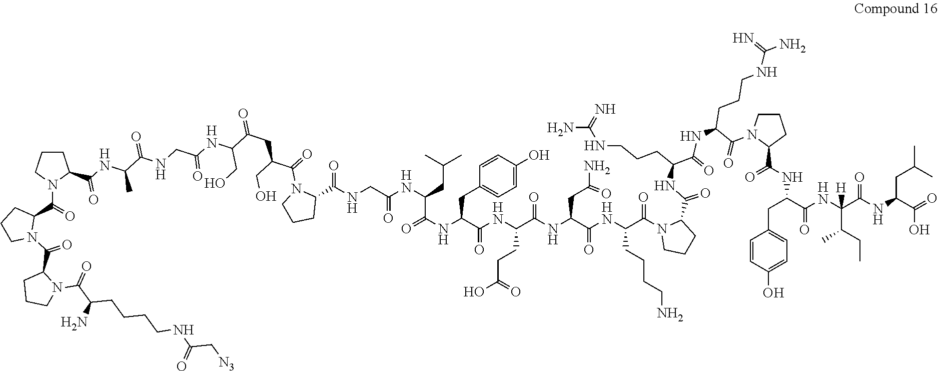 [SMILES: CC[C@H](C)[C@H](NC(=O)[C@H](Cc1ccc(O)cc1)NC(=O)[C@@H]1CCCN1C(=O)[C@H](CCCNC(=N)N)NC(=O)[C@H](CCCNC(=N)N)NC(=O)[C@@H]1CCCN1C(=O)[C@H](CCCCN)NC(=O)[C@H](CC(N)=O)NC(=O)[C@H](CCC(=O)O)NC(=O)[C@H](Cc1ccc(O)cc1)NC(=O)[C@H](CC(C)C)NC(=O)CNC(=O)[C@@H]1CCCN1C(=O)[C@@H](CO)CC(=O)C(CO)NC(=O)CNC(=O)[C@@H](C)NC(=O)[C@@H]1CCCN1C(=O)[C@@H]1CCCN1C(=O)[C@@H]1CCCN1C(=O)[C@H](N)CCCCNC(=O)CN=[N+]=[N-])C(=O)N[C@@H](CC(C)C)C(=O)O]

Compound 16: K(azidoacetyl)-PPPAGSSPGLYENKPRRPYIL: MW=1773.08, LRMS: Calcd. m/z for [M+2H]²⁺ ion of C₁₃₄H₂₃₉N₅₇O₂₆ (1771.98): 886.9. found: 885.7.

### Compound 17

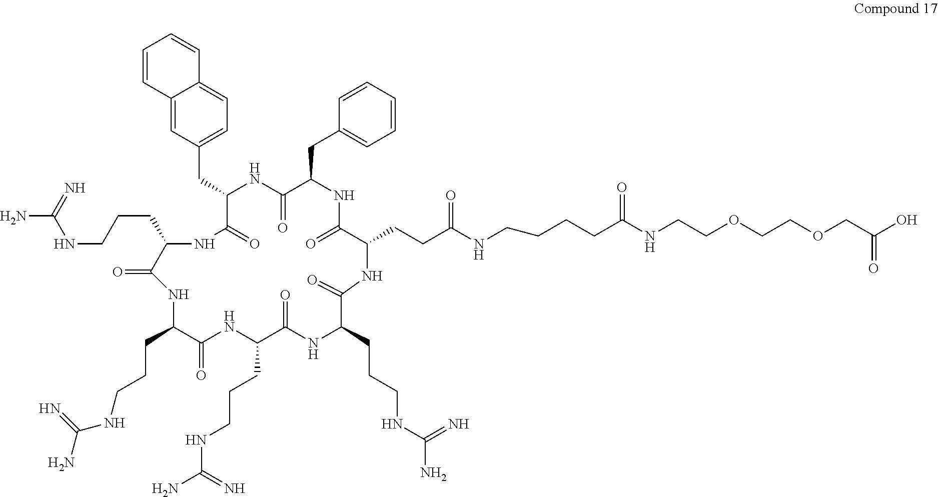 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

Compound 17—cyclo[f-[2-NaI]RrRrQ(-[Ava]-[PEG2]-acid)]: MW=1342.58, LRMS: Calcd. m/z for [M+2H]²⁺ ion of C₆₂H₉₅N₂₁O₁₃ (1341.74): 671.9. found: 672.2. f=D-phenylalanine; NaI=L-2-napthylalanine; R=L-arginine, r=D-arginine, Ava=5-aminovaleric acid (5-aminopentanoic acid).

### Compound 18

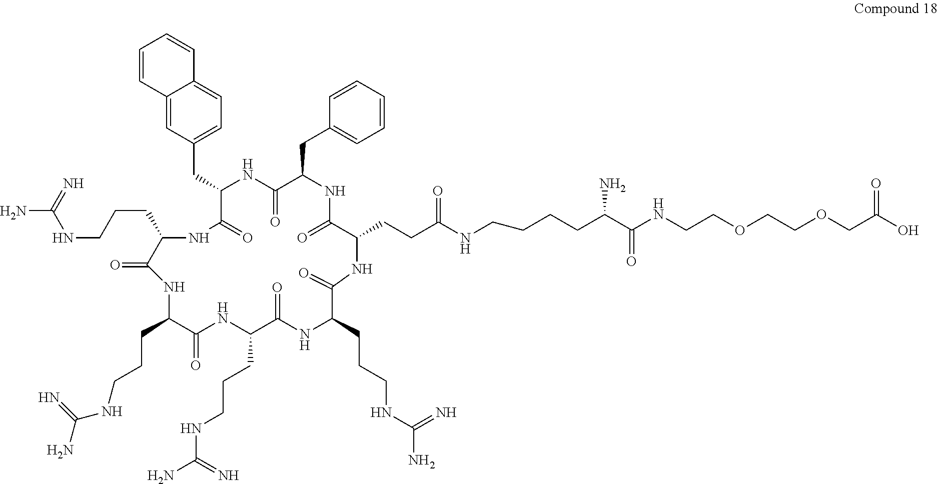 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCC[C@H](N)C(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

Compound 18: MW=1370.11, LRMS: Calcd. m/z for [M+2H]²⁺ ion of C₆₃H₉₈N₂₂O₁₃ (1371.62): 686.8. found: 686.7.

### Compound 19

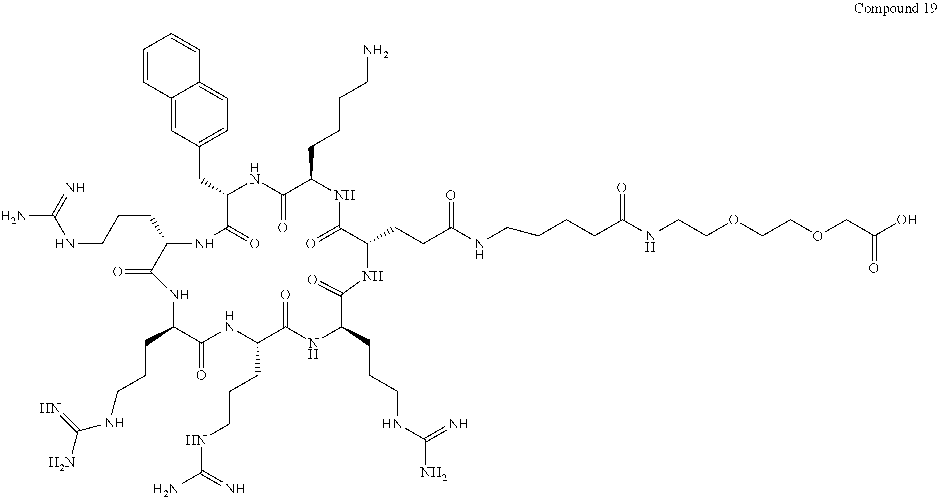 [SMILES: N=C(N)NCCC[C@@H]1NC(=O)[C@H](Cc2ccc3ccccc3c2)NC(=O)[C@@H](CCCCN)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

Compound 19: MW=1322.77, LRMS: Calcd. m/z for [M+2H]²⁺ ion of C₅₉H₉₈N₂₂O₁₃ (1323.57): 662.8. found: 662.6.

### Compound 20

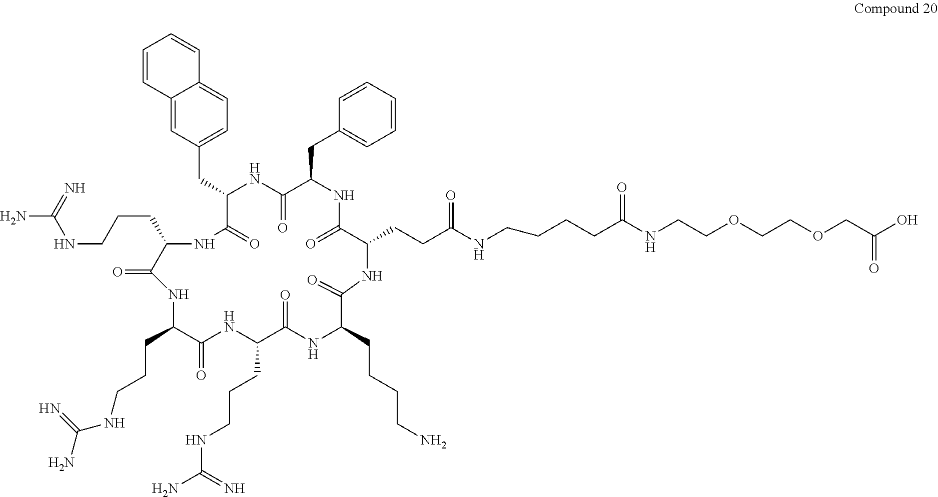 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCCN)NC(=O)[C@H](CCCNC(=N)N)NC(=O)[C@@H](CCCNC(=N)N)NC1=O]

Compound 20: MW=1313.74, LRMS: Calcd. m/z for [M+2H]²⁺ ion of C₆₂H₉₅N₁₉O₁₃ (1314.56): 657.3. found: 658.1.

### Example 14: General Procedure C for Attachment of (R)-Lipoic Acid (LA) to Compounds 18, 19 and 20

A solution of (R)-lipoic acid-NHS ester (3 equiv, in NMP 10 L/mg peptide) and DIPEA (7 equiv; in 10 μL NMP/mg peptide) was added to either Compound 18, 19 or 20 (1 equiv) in NMP (10 μL/mg). The reaction was stirred overnight and was quenched with water (10 eq) in NMP upon completion by UPLC. The solution was diluted with MTBE, the solid was centrifuged, and the pellet was washed with MTBE. The product was observed by UPLC and the desired mass was identified. The product was used in Example 16 with no further purification.

### Compound 21

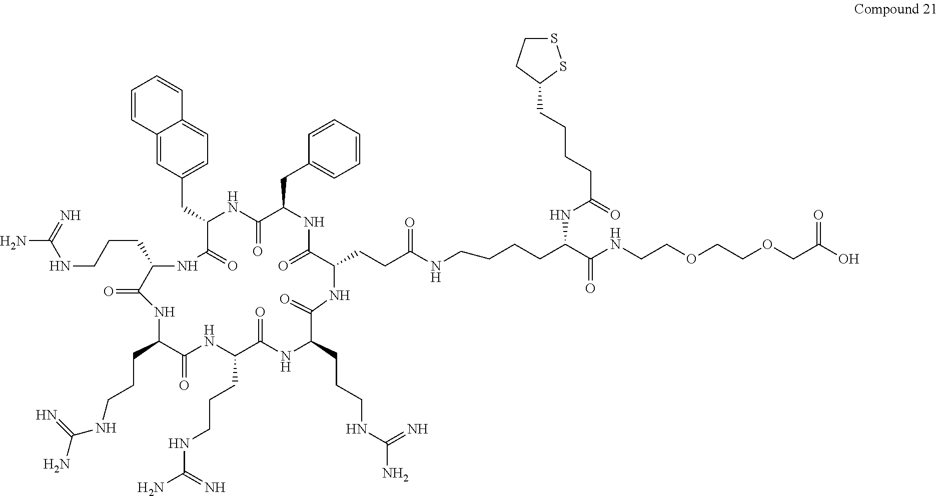 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCC[C@H](NC(=O)CCCC[C@@H]2CCSS2)C(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

Compound 21: (5.4 mg, 47%) prepared according to General Procedure A for 18 (10 mg). MW=1558.80, LRMS: Calcd. m/z for [M+2H]²⁺ ion of C₇₁H₁₁₀N₂₂O₁₄S₂ (1559.92): 780.0. found: 780.5.

### Compound 22

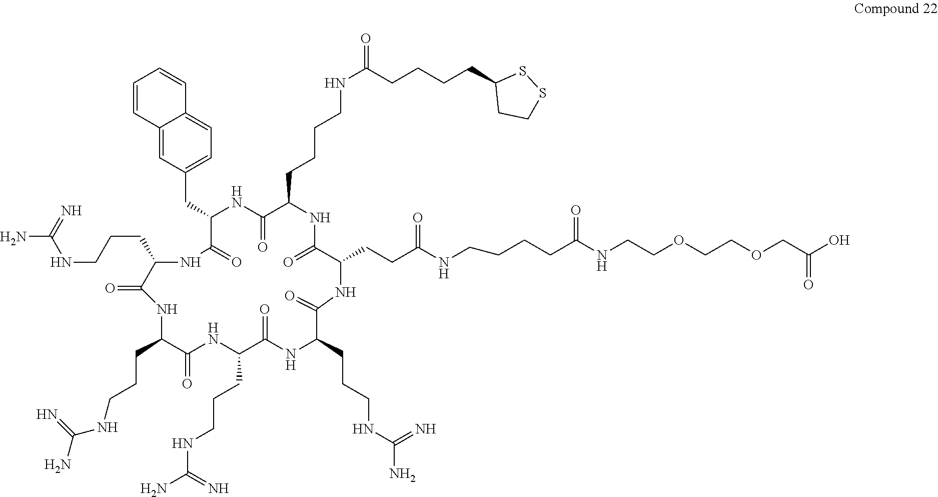 [SMILES: N=C(N)NCCC[C@@H]1NC(=O)[C@H](Cc2ccc3ccccc3c2)NC(=O)[C@@H](CCCCNC(=O)CCCC[C@@H]2CCSS2)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

Compound 22: (10.2 mg, quantitative) prepared according to General Procedure A for 19 (10 mg). MW=1510.8, LRMS: Calcd. m/z for [M+2H]²⁺ ion of C₆₇H₁₁₀N₂₂O₁₄S₂ (1511.9): 755.9. found: 756.6.

### Compound 23

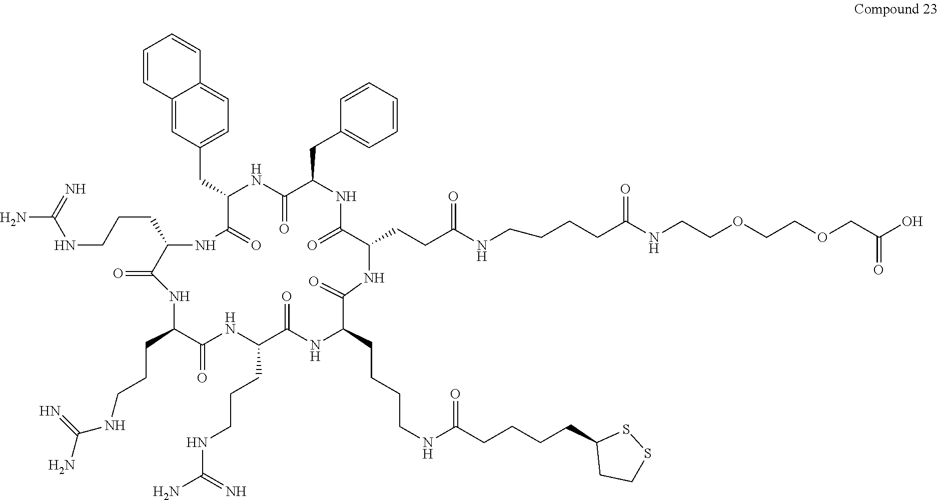 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCCNC(=O)CCCC[C@@H]2CCSS2)NC(=O)[C@H](CCCNC(=N)N)NC(=O)[C@@H](CCCNC(=N)N)NC1=O]

Compound 23 (5.9 mg, 51%) prepared according to General Procedure A for 20 (10 mg). MW=1501.77, LRMS: Calcd. m/z for [M+2H]²⁺ ion of C₇₀H₁₀₇N₁₉O₁₄S₂ (1502.87): 751.4. found: 752.1.

### Example 15: General Procedure D for Synthesis of Peptide-PMO Conjugates by Click Reaction

Compound 9 (1.0 equiv) or Compound 10 (1.0 equiv), water (30 vols), and Et₃NHOAc (3 vols, 1.0M solution in water) were added to a vial. A Peptide-containing azide (amount as listed per specific example) was dissolved in DMSO (10 vols) and added to the reaction mixture. Upon completed reaction as monitored by HPLC-MS (high-performance liquid chromatography-mass spectrometry), MeCN (40-100 vols) was added to form a white precipitate. The mixture was centrifuged to compact the solid and the supernatant was decanted. The solid was purified by size-exclusion chromatography using the general purification conditions in Table 25. The obtained fractions were desalted, filtered over a sterilizing Paal Mustang® 0.2 μm syringe filter, and lyophilized to afford the peptide-PMO conjugate as a white solid. Products of click reaction contain mixture of triazole regioisomers.

TABLE 25
General purification conditions of peptides
by size exclusion chromatography

Column Type: Superdex 75 Increase
10/300 GL (max 3 MPa)
Mobile phase: 1 × PBS, PH 7.4
Injection volume: 500 μl
Detector: 280 nm
Flow rate: 0.8-0.9 ml/min
Fraction size: 500 μl

### Compound 24

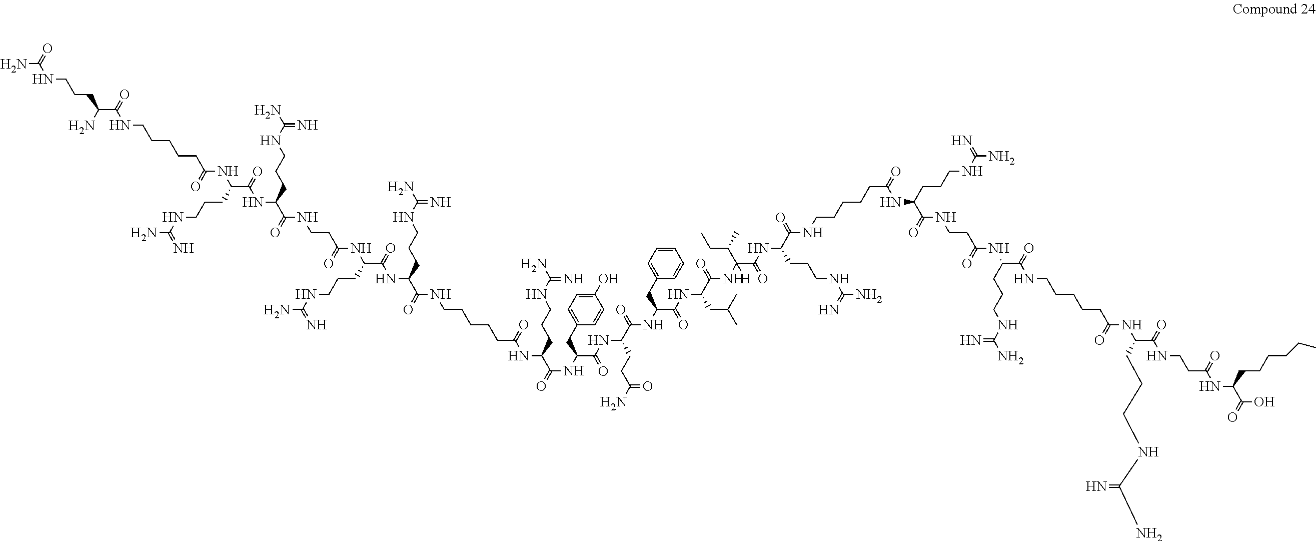 [SMILES: CCCCCC[C@H](NC(=O)CCNC(=O)[C@H](CCCNC(=N)N)NC(=O)CCCCCNC(=O)[C@H](CCCNC(=N)N)NC(=O)CCNC(=O)[C@H](CCCNC(=N)N)NC(=O)CCCCCNC(=O)[C@H](CCCNC(=N)N)NC(=O)C(NC(=O)[C@H](CC(C)C)NC(=O)[C@H](Cc1ccccc1)NC(=O)[C@H](CCC(N)=O)NC(=O)[C@H](Cc1ccc(O)cc1)NC(=O)[C@H](CCCNC(=N)N)NC(=O)CCCCCNC(=O)[C@H](CCCNC(=N)N)NC(=O)[C@H](CCCNC(=N)N)NC(=O)CCNC(=O)[C@H](CCCNC(=N)N)NC(=O)[C@H](CCCNC(=N)N)NC(=O)CCCCCNC(=O)[C@@H](N)CCCNC(N)=O)[C@@H](C)CC)C(=O)O]

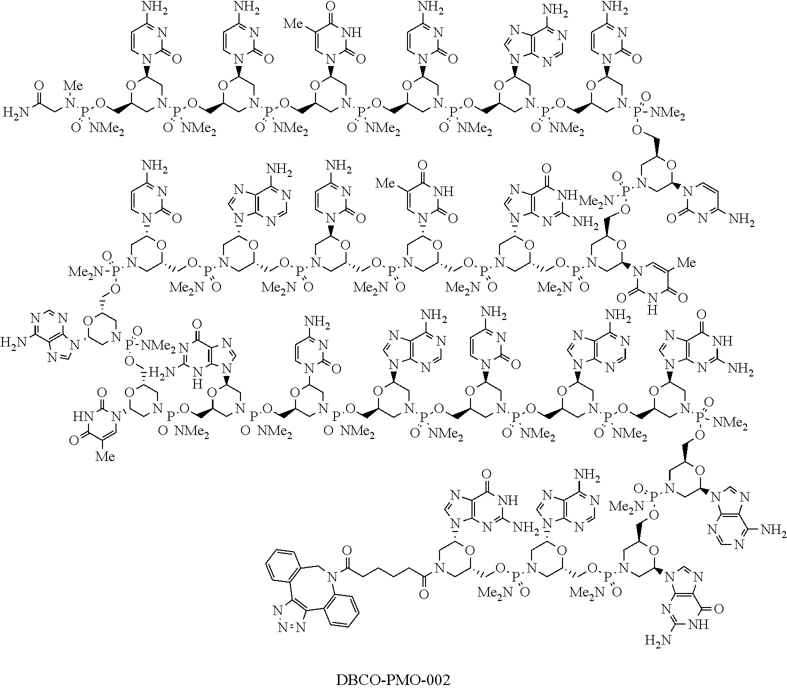

Compound 24: (5.4 mg, 53% yield) prepared by General Procedure B using Compound 9 (7.5 mg, 0.861 μmol) and Compound 12 (2.64 mg, 0.861 μmol). MW=11778.336, LRMS: Calcd. m/z for [M+16H]¹⁶⁺ ion of C₄₅₀H₇₂₀N₂₀₅O₁₂₆P₂₅ (11785.97): 737.6. found: 737.0.

### Compound 25

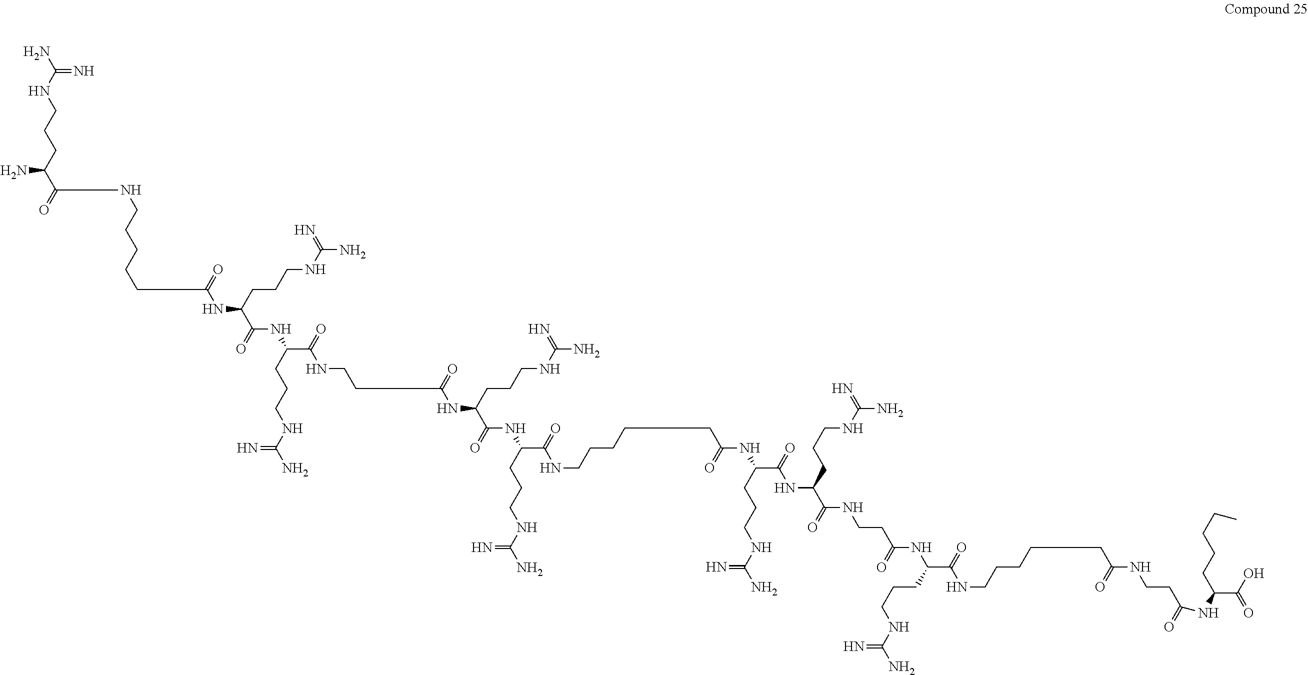 [SMILES: CCCCC[C@H](NC(=O)CCNC(=O)CCCCCNC(=O)[C@H](CCCNC(=N)N)NC(=O)CCNC(=O)[C@H](CCCNC(=N)N)NC(=O)[C@H](CCCNC(=N)N)NC(=O)CCCCCNC(=O)[C@H](CCCNC(=N)N)NC(=O)[C@H](CCCNC(=N)N)NC(=O)CCNC(=O)[C@H](CCCNC(=N)N)NC(=O)[C@H](CCCNC(=N)N)NC(=O)CCCCCNC(=O)[C@@H](N)CCCNC(=N)N)C(=O)O]

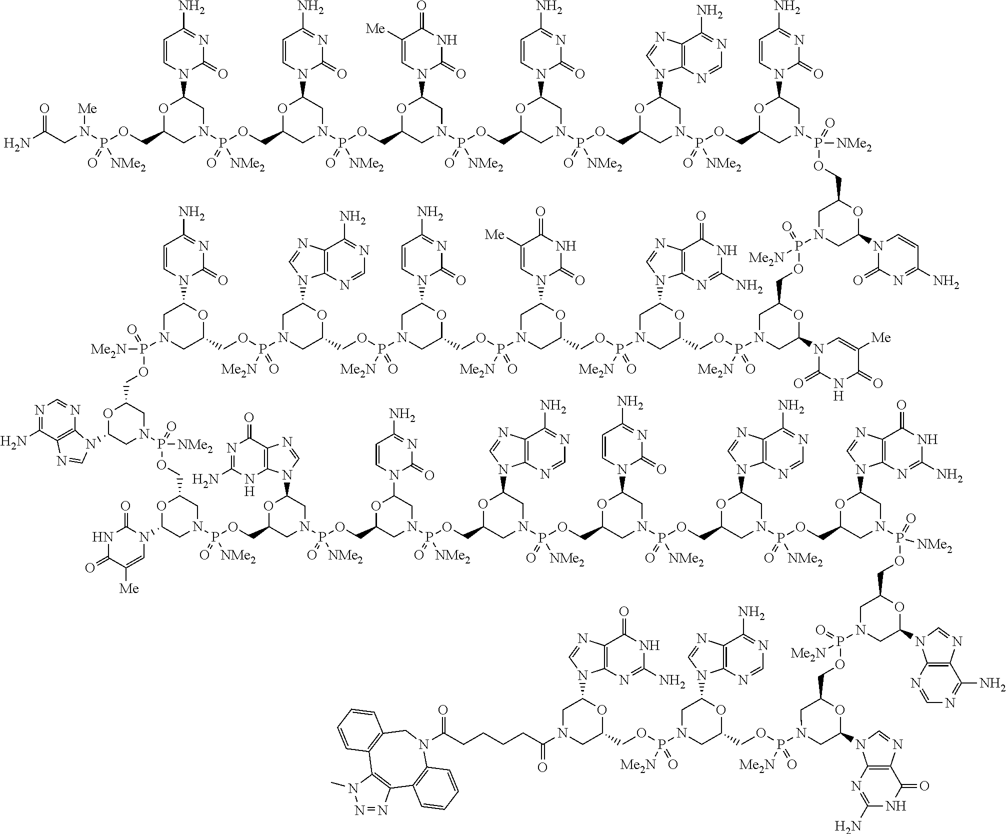

Compound 25: prepared by General Procedure B using Compound 9 (13 mg) and Compound 11 (5.89 mg). MW=10687.99, LRMS: Calcd. m/z for [M+12H]¹²⁺ ion of C₃₉₆H₆₃₅N₁₉₀O₁₁₆P₂₅ (10682.31): 891.2. found: 891.4.

### Compound 26

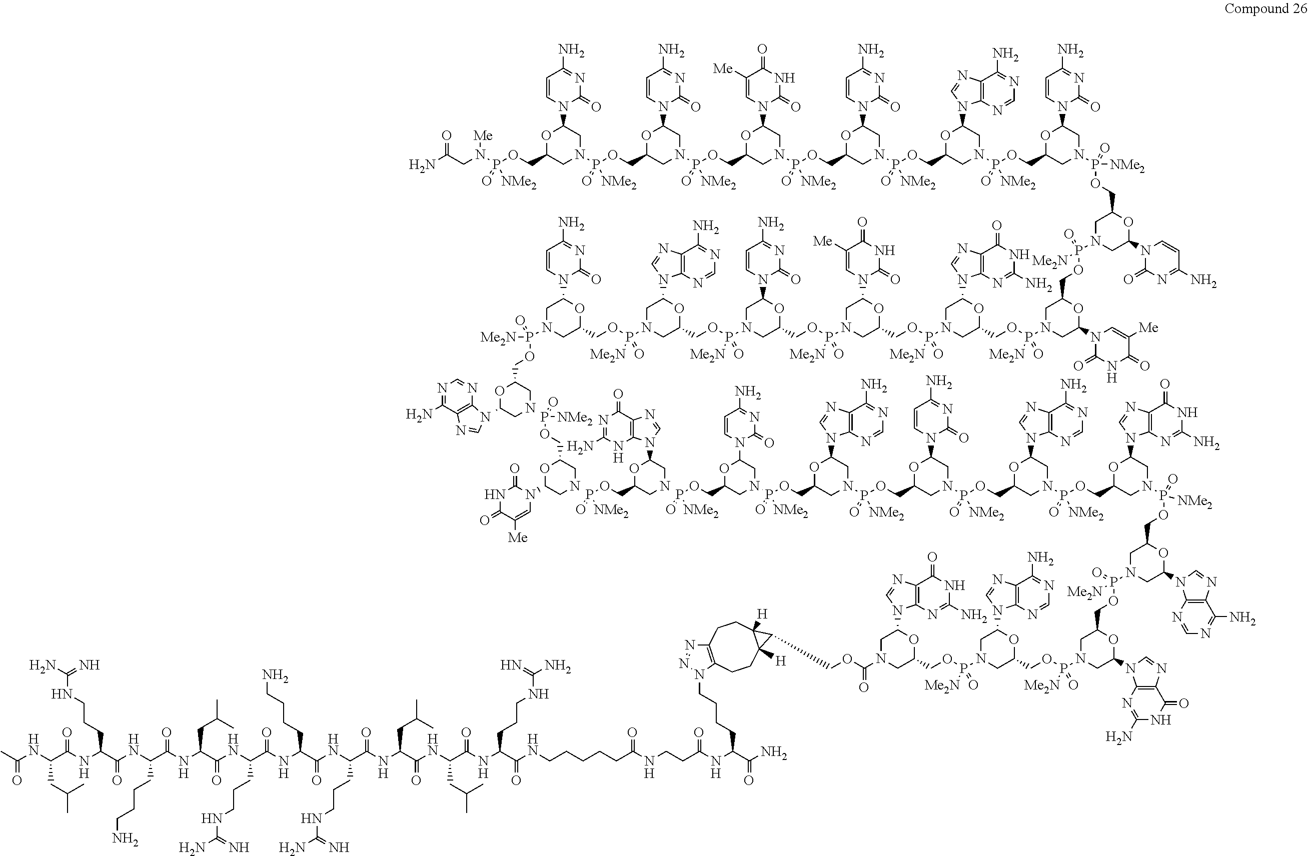

Compound 26: prepared by General Procedure B from Compound 10 (12 mg) and Compound 14 (7.3 mg). MW=10305.65, LRMS: Calcd. m/z for [M+11H]¹¹⁺ ion of C₃₈₂H₆₂₁N₁₇₈O₁₁₄P₂₅ (10300.17): 937.4; found: 938.0.

### Compound 27

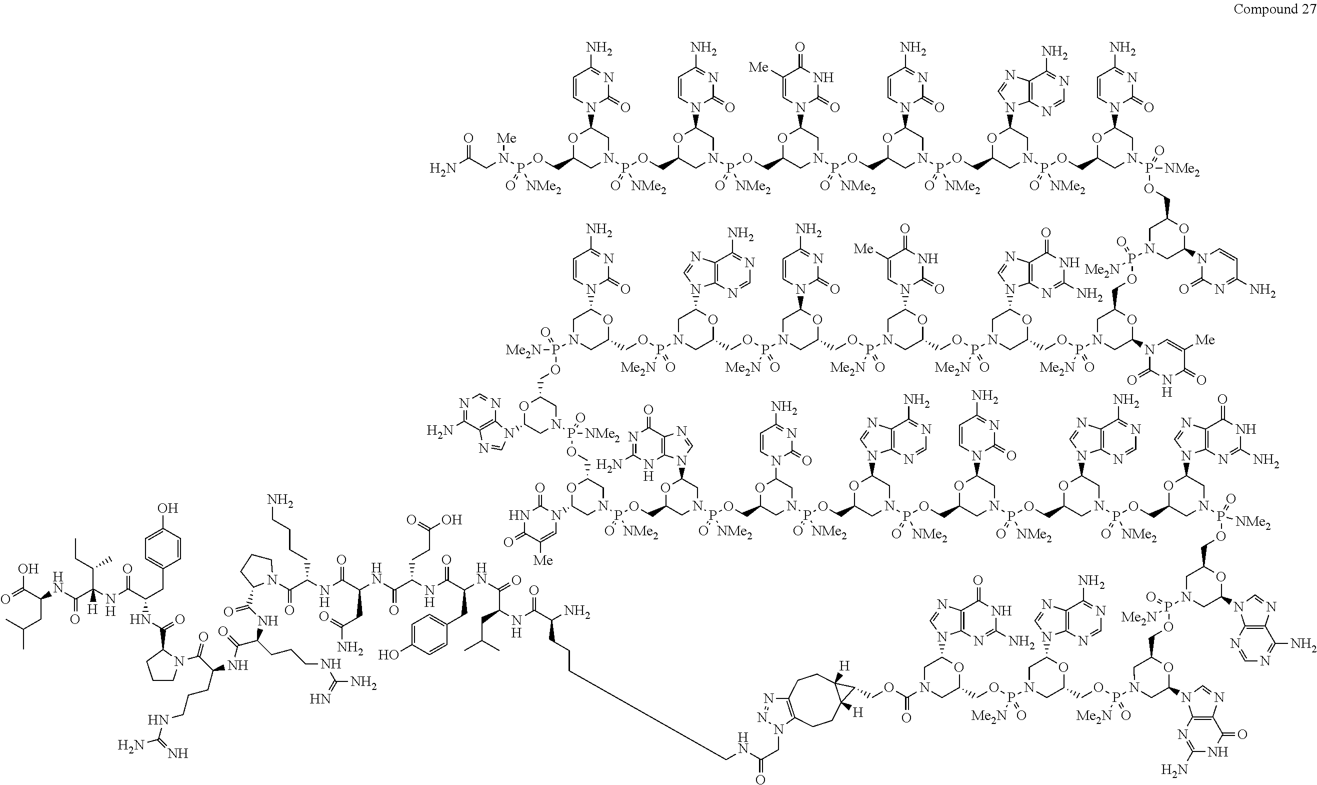

Compound 27: 4.4 mg (46% yield) prepared by General Procedure B from Compound 10 (8 mg) and Compound 15 (4.9 mg). MW=10347.98, LRMS: Calcd. m/z for [M+8H]⁸⁺ ion of C₃₈₆H₆₀₃N₁₇₂O₁₂₀P₂₅ (10341.98): 1293.7. found: 1294.3.

### Compound 28

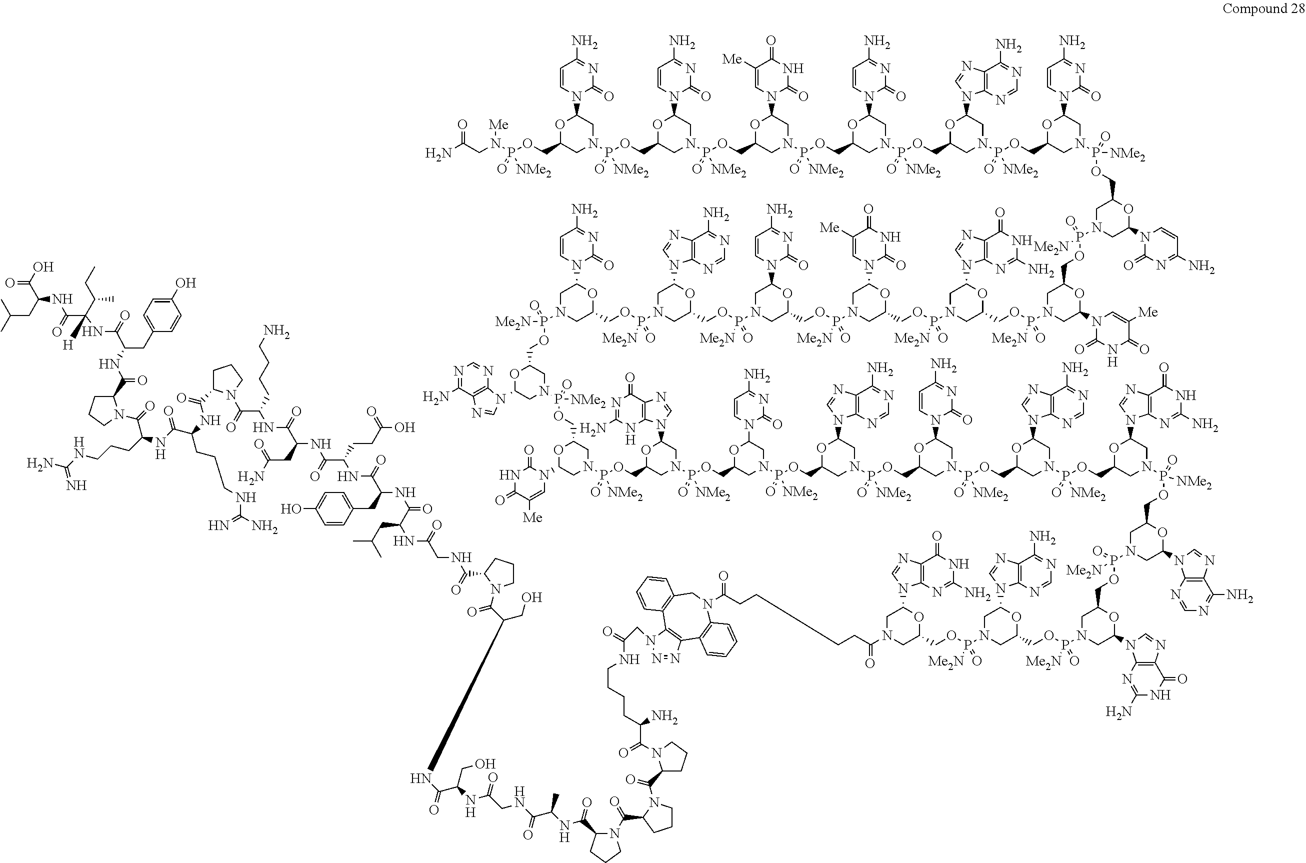

Compound 28: 2.0 mg (31% yield) prepared by General Procedure B from Compound 9 (5 mg) and Compound 16 (4.7 mg). MW=11234.46, LRMS: Calcd. m/z for [M+9H]⁹⁺ ion of C₄₂₉H₆₅₇N₁₈₂O₁₃₁P₂₅ (11228.38): 1248.6. found: 1249.1.

### Example 16: General Procedure E for Peptide-PMO Conjugate by Amide Bond

29: PMO-002 (12 mg, 1.43 μmol) and DMSO (100 μL) were added to a first vial and the suspension was warmed to 37 °C until a clear solution was formed. 13 (7.5 mg, 2.57 μmol, 1.8 equiv) and N-methylpyrrolidinone (50 μL) were added to a second vial. HOBT (0.5 mg, 2.8 μmol, 2.0 equiv.), Hunig's base (1 μL, 5 μmol, 3.5 equiv) and N-methylpyrrolidinone (50 μL) were added to the second vial and mixed to ensure uniformity. This solution was charged with HBTU (1.4 mg, 3.57 μmol, 2.5 equiv.), mixed on a vortex to achieve dissolution, and after 1 minute the solution was transferred via syringe into the first vial containing PMO-002 in DMSO. The vial was sealed and the heated in microwave at 65 °C for 15 minutes. Upon coaling to RT, the product was precipitated by addition of MeCN (9 mL). The solids were collected by decanting, dissolved in water, and purified by general procedure F for ion exchange chromatography using Resource S column as shown in Table 26. The collected fractions were desalted and lyophilized to afford the product (3 mg).

TABLE 26
General procedure F for purification Compounds
29, 30 and 31 by ion-exchange chromatography

Column: Resource S (6 ml)
Buffer A: 1 × PBS pH 7.4, 25% acetonitrile;
Buffer B: 1 × PBS pH 7.4, 0.9M NaCl,
25% acetonitrile
Gradient: 0-50% buffer B in 11 CV (66 ml);
0-100% buffer B in 22 CV (132 ml)
Injection volume: 500 μl-1 ml
Detector: 280 nm; Flow rate: 4 ml/min;
Fraction size: 1 ml

### Compound 29

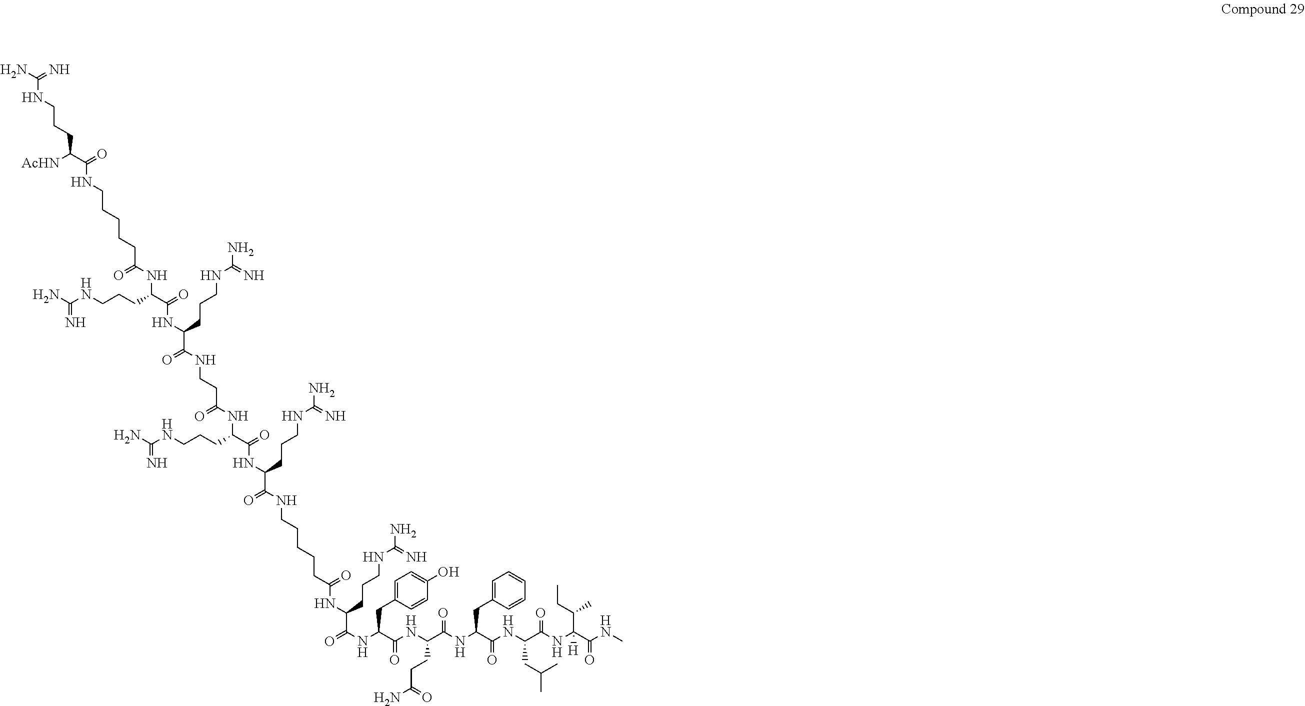 [SMILES: CC[C@H](C)[C@H](NC(=O)[C@H](CC(C)C)NC(=O)[C@H](Cc1ccccc1)NC(=O)[C@H](CCC(N)=O)NC(=O)[C@H](Cc1ccc(O)cc1)NC(=O)[C@H](CCCNC(=N)N)NC(=O)CCCCCNC(=O)[C@H](CCCNC(=N)N)NC(=O)[C@H](CCCNC(=N)N)NC(=O)CCNC(=O)[C@H](CCCNC(=N)N)NC(=O)[C@H](CCCNC(=N)N)NC(=O)CCCCCNC(=O)[C@H](CCCNC(=N)N)NC(C)=O)C(=O)NC]

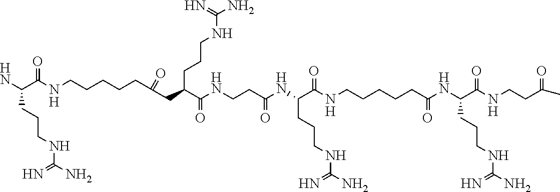

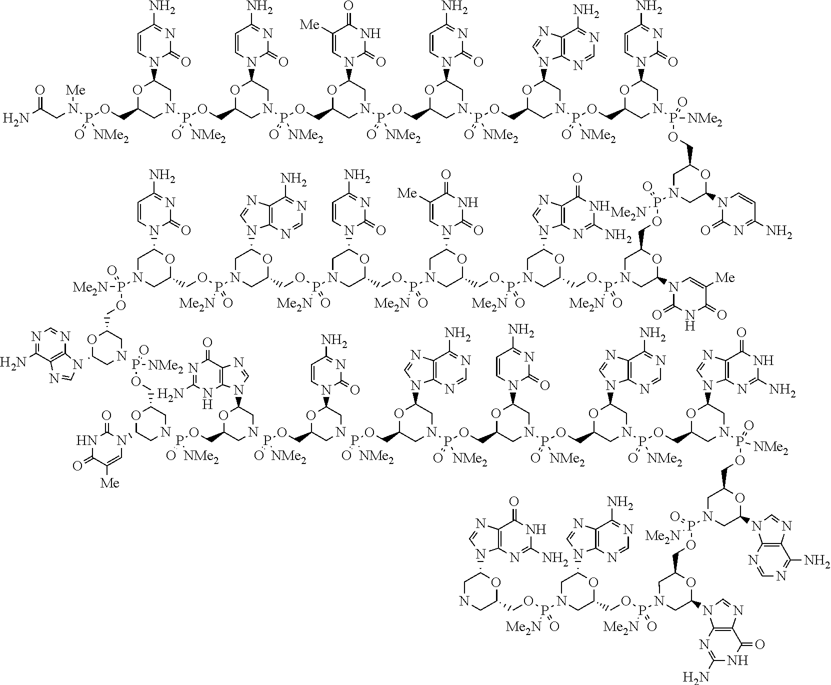

Compound 29: MW=11332.81, LRMS: Calcd. m/z for [M+9H]⁹⁺ ion of C₄₂₄H₆₉₁N₂₀₀O₁₂₃P₂₅ (11326.74): 872.3. found: 872.8.

### Compound 30

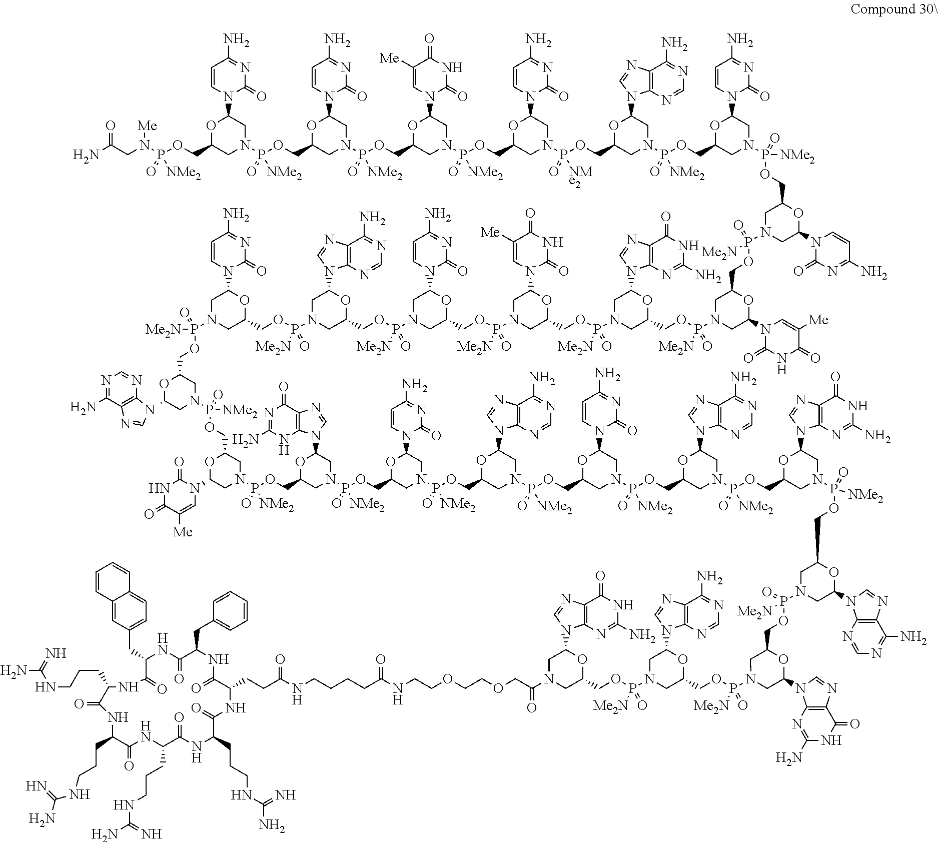

Compound 30: (5.3 mg, 46%) prepared according to General Procedure E using PMO-002 (10 mg) and Compound 20 (6.4 mg). MW=9722.77, LRMS: Calcd. m/z for [M+10H]¹⁰⁺ ion of C356H555N1680110P25 (9717.64): 972.8; found: 973.3.

### Compound 31

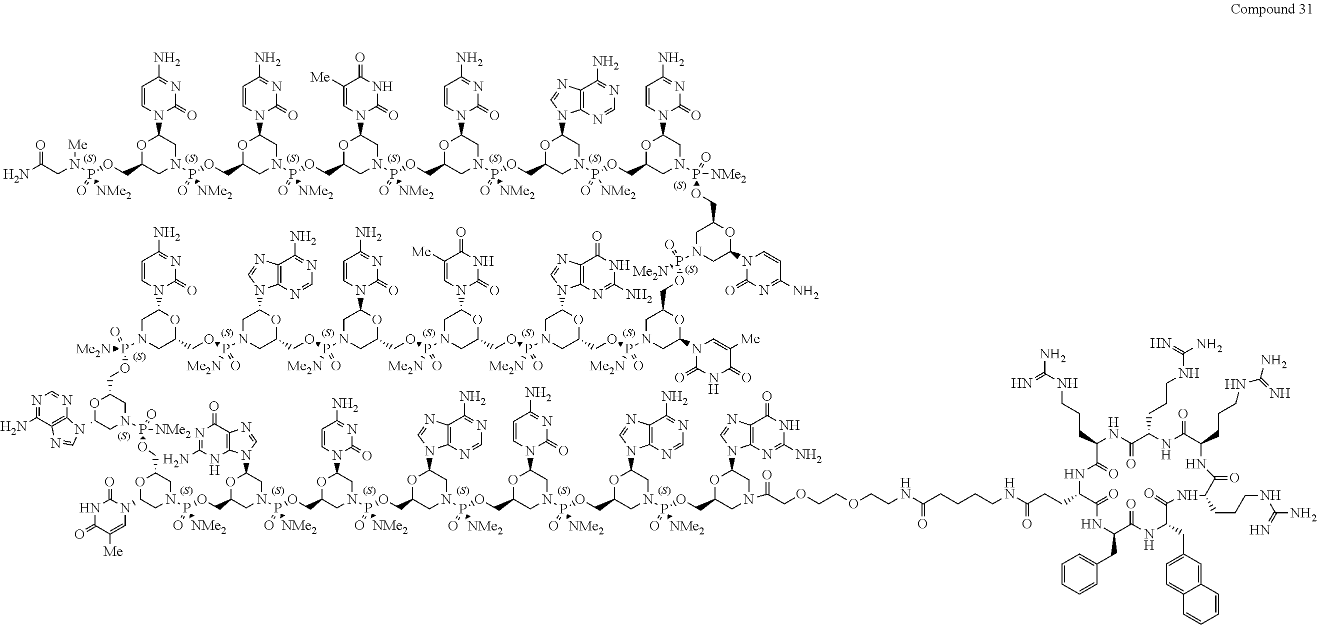

Compound 31: (7.3 mg, 61%) prepared according to General Procedure E using (Sp) PMO-424 (10 mg) and Compound 17 (7.6 mg). MW=8333.59, LRMS: Calcd. m/z for [M+10H]¹⁰⁺ ion of C308H483N140096P21 (8329.17): 833.9; found: 834.3.

TABLE 27
General procedure G for purification of Compounds
32, 33 and 34 by ion-exchange chromatography.

Column: Resource S (6 ml)
Buffer A: 20 mmol HEPES 7.4, 10% acetonitrile;
Buffer B: 20 mmol HEPES 7.4, 10
% acetonitrile, 1M NaCl
Gradient: 0-10% buffer B in 2 CV (12 ml);
10-35% buffer B in 2 CV (12 ml); 35-100%
buffer B in 22 CV (48 ml)
Injection volume: up to 500 μl
Detector: 280 nm; Flow rate: 4 ml/min;
Fraction size: 1 ml

### Compound 32

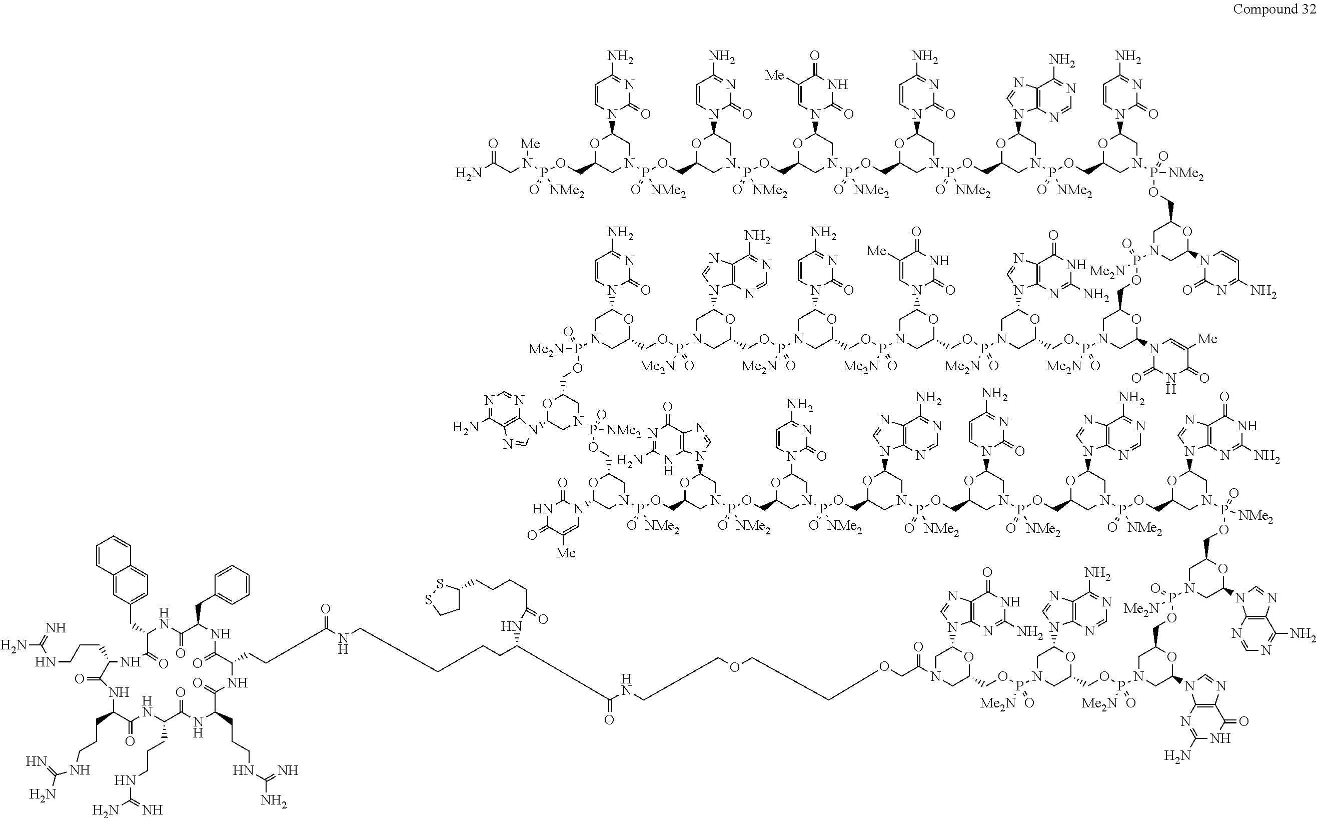

Compound 32: (0.180 mg, 1.5%) prepared according to General Procedure E using PMO-002 (10 mg) and Compound 21 (5.4 mg). Purified with general procedure G. MW=9940.1, LRMS: Calcd. m/z for [M+10H]¹⁰⁺ ion of C₃₆₅H₅₇₀N₁₆₉O₁₁₁P₂₅S₂ (9934.70): 994.5. found: 994.0.

### Compound 33

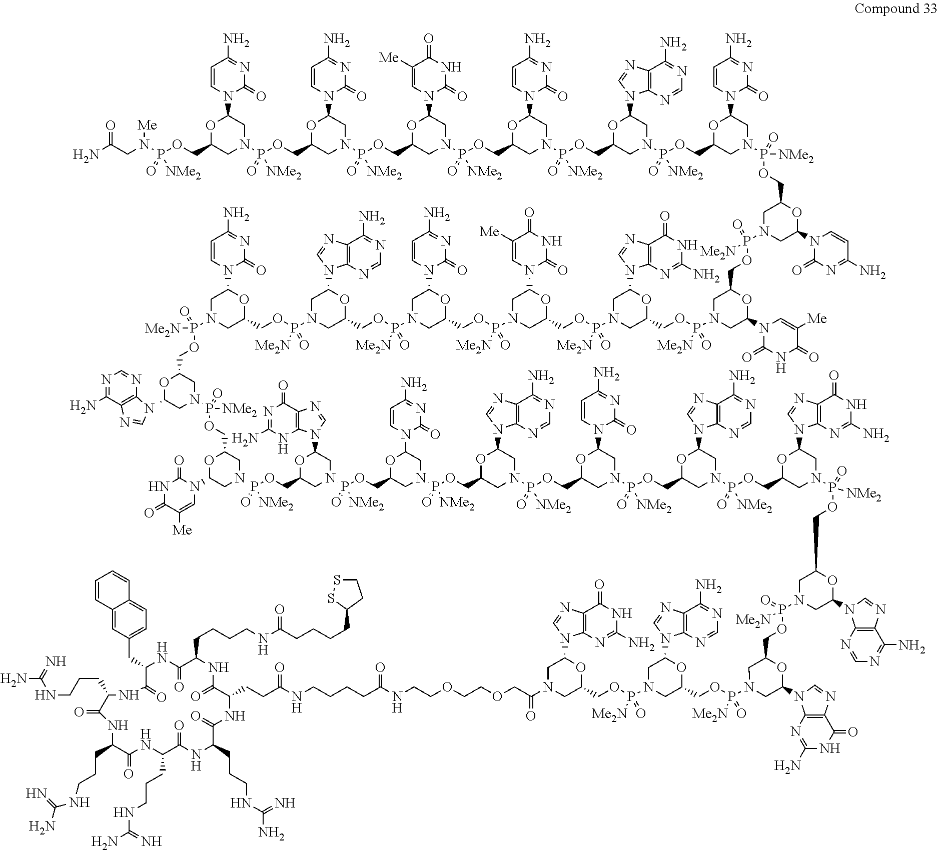

Compound 33: (4.3 mg, 36%) prepared according to General Procedure E using PMO-002 (10 mg) and Compound 22 (10.0 mg). Purified with general procedure G. MW=9892.1, LRMS: Calcd. m/z for [M+10H]¹⁰⁺ ion of C₃₆₁H₅₇₀N₁₈₉O₁₁₁P₂₅S₂ (9886.70): 989.7. found: 989.9.

### Compound 34

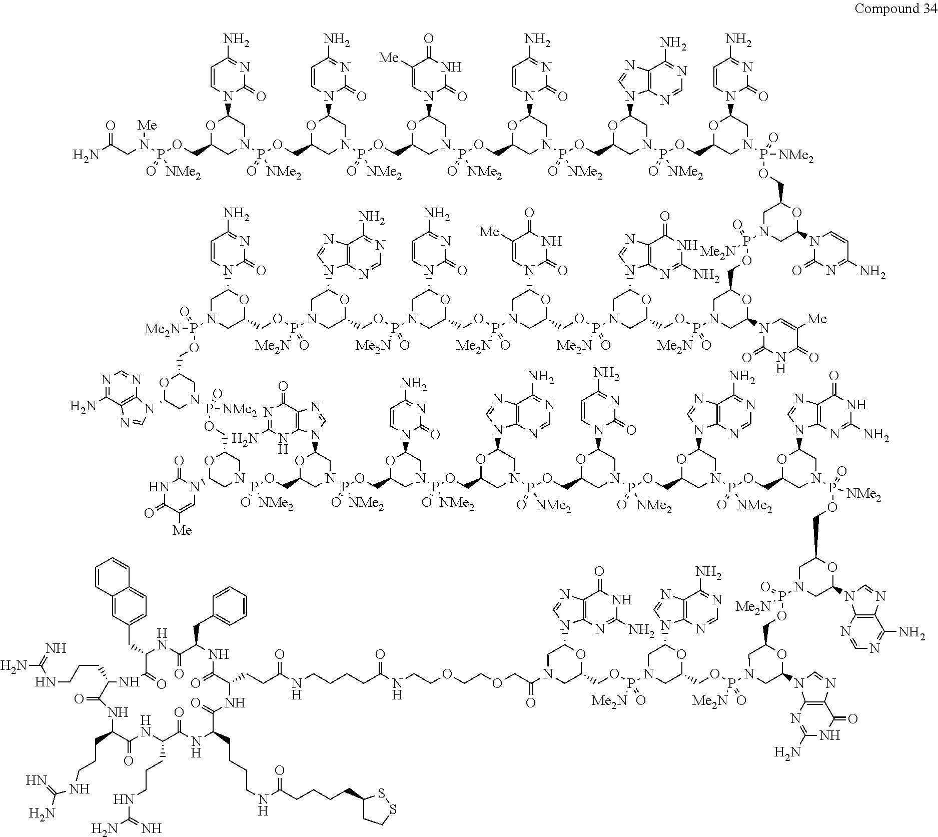

Compound 34: (0.340 mg, 2.9%) prepared according to General Procedure E using PMO-002 (10 mg) and Compound 23 (5.9 mg). Purified with general procedure G. MW=9883.1, LRMS: Calcd. m/z for [M+10H]¹⁰⁺ ion of C₃₆₃H₅₆₅N₁₆₆O₁₁₁P₂S₂ (9877.67): 988.8. found: 989.1.

### Example 17: Exon-Skipping Efficiency Assay in Mouse Bone-Marrow Derived Macrophages (mBMDM) Cells In Vitro

Freshly isolated mBMDM cells from humanized-CD33 mice were cultured and maintained using Dulbecco's Modified Eagle's Medium containing 10% fetal bovine serum with recombinant murine CSF. The Exon-Skipping Efficiency Assay was performed in 96 well plate format, seeding about 50,000 cells per well and treating with each peptide-PMO ASO at a concentration of 0.078 μM, 0.156 μM, 0.31 μM, 0.63 μM, 1.25 μM, 2.5 μM, and 10 μM without addition of transfection reagents. Cells were incubated at 37 °C in a cell culture incubator for 48 hours before isolating the total RNA. Total RNA was isolated and converted to cDNA per vendor protocol, then Taqman gene expression assays were used to quantify Exon-2 skipped CD33 (Forward primer: CGCTGCTGCTACTGCTG (SEQ ID NO:207); Reverse Primer: TTCTAGAGTGCCAGGGATGA (SEQ ID NO:208); and probe: TGTGGGCAGACTTGACCCACAG (SEQ ID NO:209)) and un-skipped CD33 (Forward primer: GGATGGAGAGAGGAAGTA (SEQ ID NO:210); Reverse Primer GTGCCAGGGATGAGGATTT (SEQ ID NO:211); and probe: TGCATGTGACAGACTTGACCCACA (SEQ ID NO:212)) mRNA transcripts. Mouse house-keeping gene HPRT1 was used to normalize the target transcript expressions.

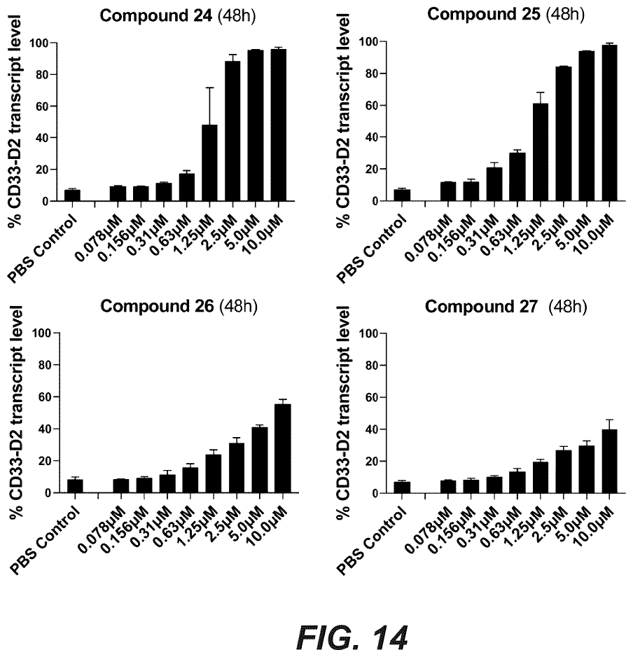

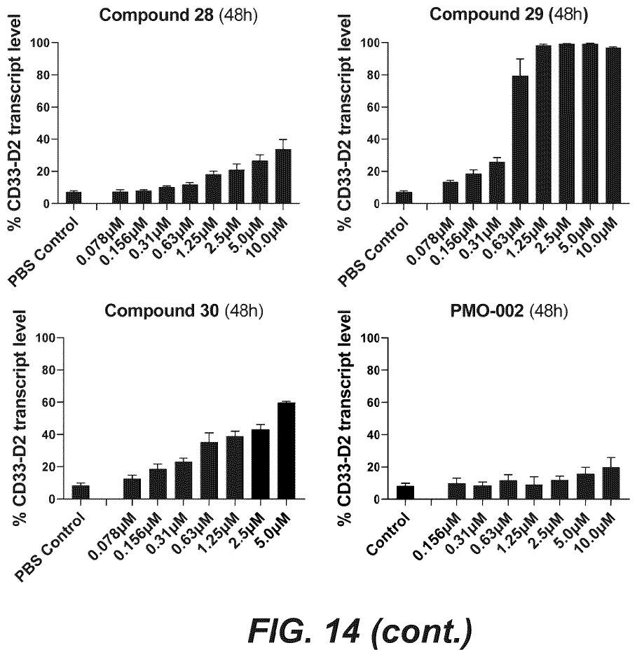

Selected conjugates were tested in mBMDMs for CD33 D2 skipping efficiency under gymnotic (non-transfection) conditions. As shown in FIG. 14, all tested conjugates showed improved activity when compared to naked PMO-002.

Different technologies can be used to assess the activity/properties of CD33 targeting oligonucleotides using various human, mouse, and non-human primate cell lines.

### Example 18: In Vivo Assays

Exon Skipping Activity Assay in a hCD33 Mouse Model Whole Brain after Intracerebroventricular Administration.

Humanized CD33 mouse models were used to study CD33 Exon-2 skipping CPP-ASOs in vivo. CRISPR/Cas9 mediated gene editing was used to replace murine CD33 with human genomic CD33, including the signal peptide. Murine 3′ and 5′ untranslated regions were retained. For the in vivo assays, mixed gender cohorts of human CD33 mouse lines on a C57BL/6 background were used. Mice were 12-24 weeks old at the time of dosing. CPP-ASOs were administered as solution in PBS via intracerebroventricular (ICV) injection into the right lateral ventricle in a 10 μL bolus injection on day 1. Mice were necropsied 1 week after the injection, or longer in the case of a duration study. At necropsy, mice were transcardially perfused with PBS under avertin anesthesia. Brains were rapidly removed from the skull, and the cortex and hippocampus were dissected from the injected hemisphere for use in exon skipping evaluation. For RNA isolation, frozen tissue was added with 9× volume of Trizol and homogenized for 3 minutes. 500 μL of the Trizol lysate was transferred to a 1 mL deep well plate. 100 μL of chloroform was added to each sample, shaken vigorously, and centrifuged at 4000×g for 5 minutes. The supernatant (250 μL) was transferred to the binding plate from SV96 total RNA extraction kit (Promega) and RNA was extracted per the same protocol. Total RNA was isolated and converted to cDNA per SV96 protocol (Promega), then Taqman gene expression assays were used to quantify Exon-2 skipped CD33 mRNA transcripts. Mouse house-keeping gene HPRT1 was used to normalize the target transcript expressions.

Determining Concentration of CPP-PMO Conjugates in Mouse Cortex and Hippocampus using HELISA

Compounds 30, 31 and 33 were quantified in mouse cortex and hippocampus using a hybridization-based immunoassay method (HELISA). Tissues were lysed in TRIzol, 1:10 (Thermo Fisher Scientific, Waltham, MA), and were diluted in hybridization buffer (1:100, 1M NaCl in TE-Buffer and 0.1% Tween® 20). Compound 30 was spiked in diluted tissue homogenate to prepare standard curves and quality controls (QC). 35 μL of diluted samples, standards, and QCs were transferred to a 96-well PCR plate. 35 μl of detection probe solution (5′-GTGACAGGTGAGG/3Bio/-3′ (for compound 30, 33, Integrated DNA Technologies, Inc, Coralville, IA), 5′-/5DigN/CTGTGCATGT-3′ (for compound 31, Integrated DNA Technologies, Inc, Coralville, IA), 100 nM in hybridization buffer), was added to the PCR plate containing standards and samples. Sample and detection probe were hybridized on a thermal cycler under the following conditions: 95 °C for 10 minutes, 37 °C for 60 minutes, and a final hold at 4 °C.

MSD Gold 96-well Streptavidin SECTOR plate (Meso Scale Diagnostics, LLC., Rockville, MD) was blocked with 150 μL of Casein in TBS blocker (Thermo Fisher Scientific, Waltham, MA) at room temperature for 1.5 hours. After washing with the wash buffer (Tris buffered saline with Tween®20, Sigma-Aldrich, St. Louis, MO), 25 μL of capture probe (5′-DigN/CTCTCTGTGCAT-3′ (for compounds 30 and 33, Integrated DNA Technologies, Inc, Coralville, IA), 5′-GACAGGTGAGG/3Bio/-3′ (for compound 31, Integrated DNA Technologies, Inc, Coralville, IA), 200 nM in hybridization buffer), was added to the MSD plate and incubated at 37 °C, 300 rpm for 1 hour. After the wash step, 25 μL of samples, standards, and QCs were transferred to the MSD plate in duplicate and were incubated at 37 °C for 1 hour on a shaking platform (300 rpm). The plate was then washed 3 times and incubated for 1 hour with 25 μL of 1 μg/mL MSD GOLD SULFO-TAG label: Anti-Digoxigenin, Fab fragment (made in-house from conjugation of MSD GOLD SULFO-TAG NHS-Ester (Meso Scale Diagnostics, LLC., Rockville, MD) with Anti-Digoxigenin, Fab fragments (Sigma-Aldrich, St. Louis) (in Casein-TBS Blocking Buffer and 0.05% Tween20. After the final wash step, 150 μL of 2×MSD Read Buffer T (Meso Scale Diagnostics, LLC., Rockville, MD) was added and the plate was read on an MSD Sector S 600 instrument (Meso Scale Diagnostics, LLC., Rockville, MD). A nonlinear regression analysis was performed to calculate the concentrations of reference compound from the signal intensities via interpolation from a calibration curve using 4-parameter logistic (4PL) model (weighting factor=1/Y2) in Discovery Workbench 4.012.1 (Meso Scale Diagnostics, LLC., Rockville, MD). The lower limit of quantitation (LLOQ) was 9.8 nM in cortex and hippocampus.

### In Vivo Results

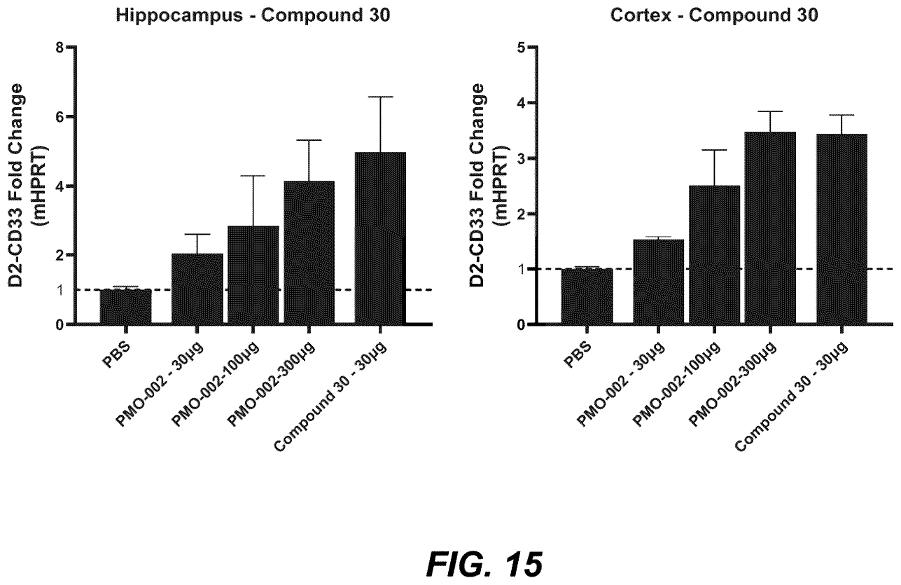

Compounds 25, 29, 30, 31, 32, 33 and 34 were dosed in vivo by ICV administration as outlined above. When dosed at 30 μg according to the in vivo assay protocol, Compounds 25 and 29 resulted in acute toxicity in mice. Hence, their in vivo Exon-2 skipping activity could not be evaluated. Compounds 30, 31, 32, 33 and 34 were tolerated. The in vivo skipping efficiency of Compound 30 is shown in FIG. 15. When dosed at 30 μg, Compound 30 showed a 10-fold improvement in skipping efficiency when compared to PMO-002 (e.g. 30 μg of Compound 30 afforded similar level of skipping as that of 300 μg of PMO-002).

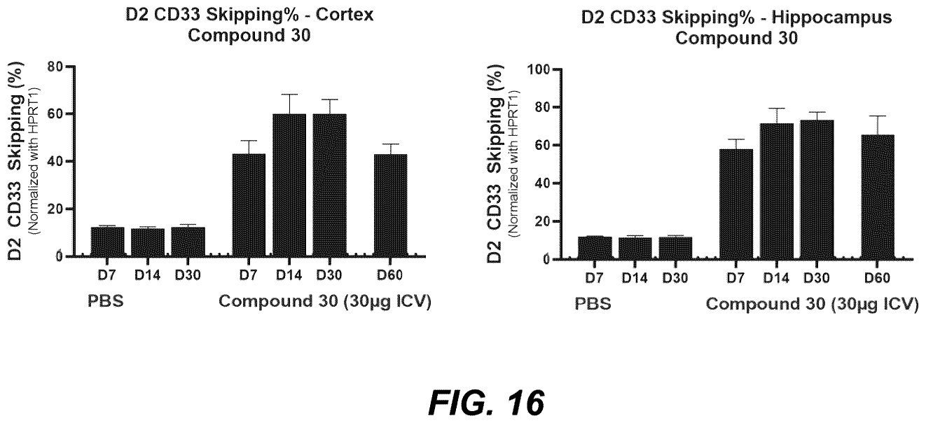

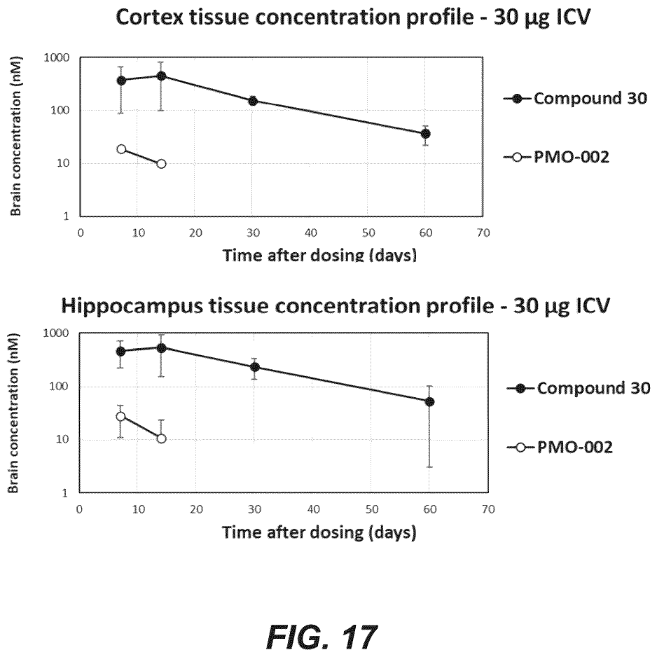

The duration of Compound 30 was also evaluated. A single 30 μg dose of Compound 30 maintained exon skipping up to 60 days in the mouse brain (cortex and hippocampus, FIG. 16). By comparison, PMO-002 showed peak activity at 7 days and declined in activity after 14 days. Analysis of the brain concentration of Compound 30 compared to PMO-002 showed a dramatically improved PK profile by higher exposure. Compound 30 had a 10-fold improvement in brain exposure relative to PMO-002 (FIG. 17).

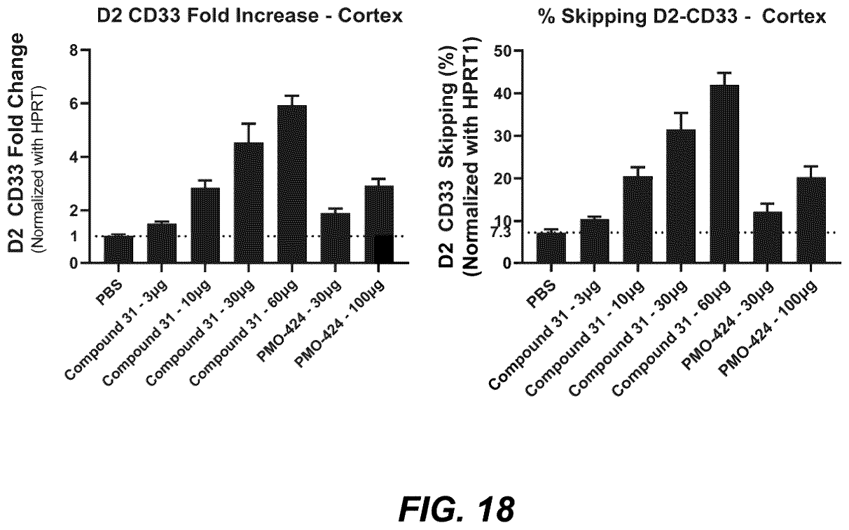

Conjugation of Sp-PMO-424 to Compound 17 created Compound 31, which had a 10-fold improvement in skipping efficacy compared to Sp-PMO-424 (FIG. 18).

Both Compound 30 and Compound 31 were tolerated in vivo by ICV administration up to the tested 60 μg dose.

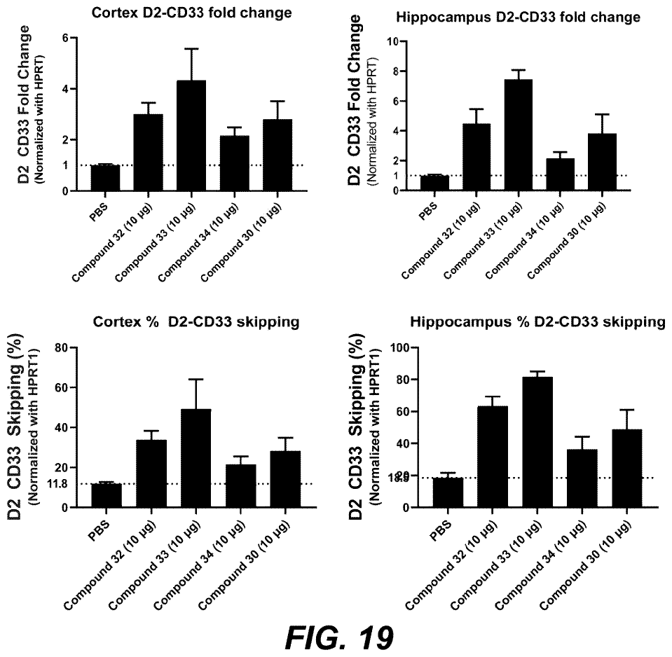

Lipoic acid contains a 5-membered disulfide ring which can increase peptide interaction with proteins and improve cellular uptake. Lipoic acid was incorporated in conjugates Compounds 32, 33, and 34 by attachment to a lysine residue. Compounds 32, 33, and 34 were tested in vivo at 10 μg doses (FIG. 19). Compound 33 contains a lysine-N-lipoic acid conjugate in the macrocyclic ring instead of the phenylalanine present in Compound 30. Compound 33 had improved skipping efficacy relative to Compounds 32 and 34, and when compared to Compound 30.

Those having ordinary skill in the art will appreciate that the disclosure can be modified in ways not specifically described herein. The disclosure is not to be limited in scope by the specific embodiments described herein, which are for illustrative purposes only. The disclosure includes any modifications and variations, including all functionally equivalent productions, compositions, and methods.

The entire disclosures of all publications cited herein are hereby incorporated by reference. No admission is made that any such publication constitutes prior art or is part of the common general knowledge of those having ordinary skill in the art.

### Example 19: Additional Examples of Cell-Penetrating Peptides

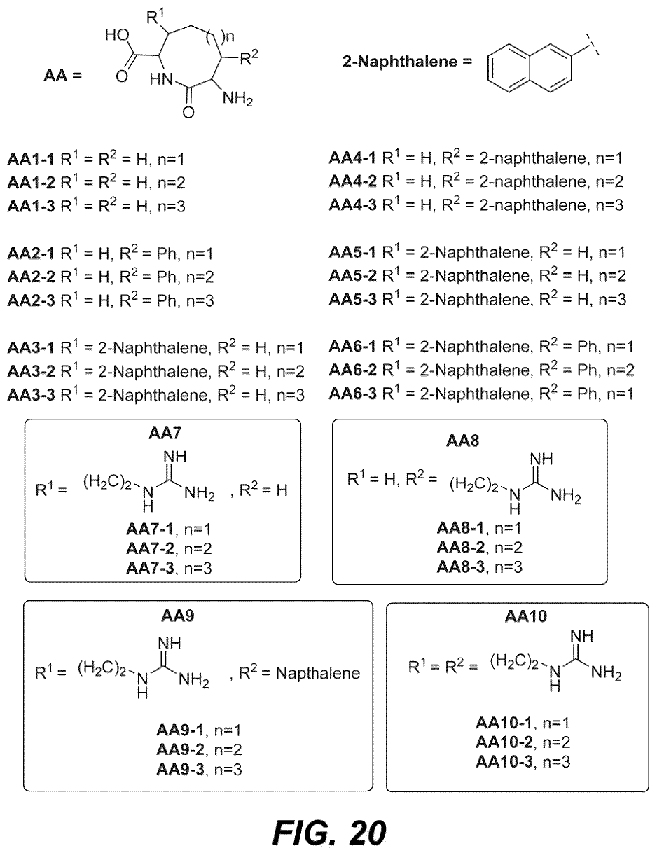

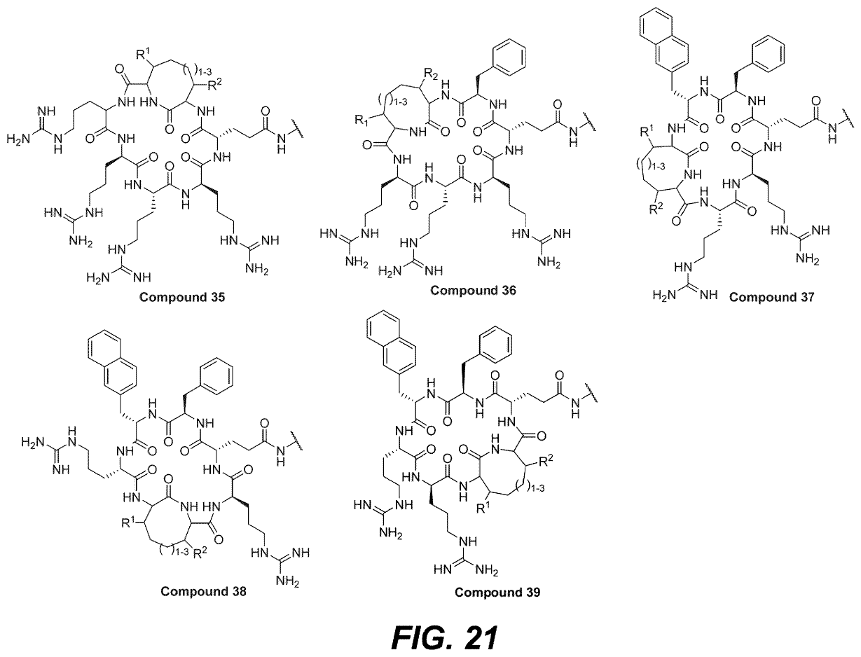

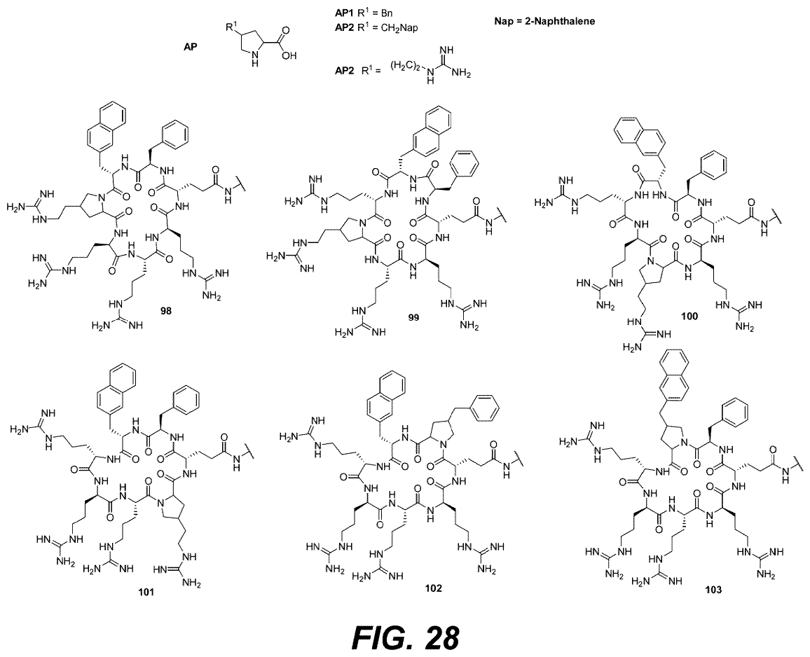

Additional examples of cell-penetrating peptides include peptides containing a cyclic lactam. In some embodiments, the cyclic lactam may contain an eight-, nine-, or ten-membered ring. In some embodiments, the cyclic lactam may be constructed such that it contains side chains (R¹, R²) with aromatic, linear or branched alkyl groups and functionalized alkyl groups. In addition, in some embodiments, the side chain may contain guanidine group such as the one found in arginine which promotes cell-penetrating activity. In some embodiments, the stereochemistry of each center may be varied accordingly to achieve the best cell-penetrating potency. Examples of lactam amino acids are listed in FIG. 20. Examples of cell-penetrating peptides with lactam amino-acids AA1-AA10 are listed in FIG. 21. In some embodiments, the synthesis of the lactam amino acids AA1-AA10 for use in peptide synthesis follows synthetic routes as illustrated in FIGS. 22-27. Additional examples of cell-penetrating peptides include peptides containing chemically modified proline residues (FIG. 28). Proline residues provide conformational bias which may not be achieved by acyclic amino acids. In some embodiments, the modified proline is constructed such that it contains side chain functional groups with aromatic, linear, and/or branched alkyl groups. In addition, in some embodiments, the side chain contains one or more guanidine group such as the one found in arginine. In some embodiments, the stereochemistry of each center may be varied accordingly to achieve the best cell-penetrating potency. For Examples 19-47, if a compound does not depict a specific stereochemical configuration, it includes every possible stereochemical configuration.

In some embodiments, synthesis of proline modified with a guanidine side chain is conducted according to a previously reported method (Ishiguro et al. J. Med. Chem. 2004, 47, 489-492).

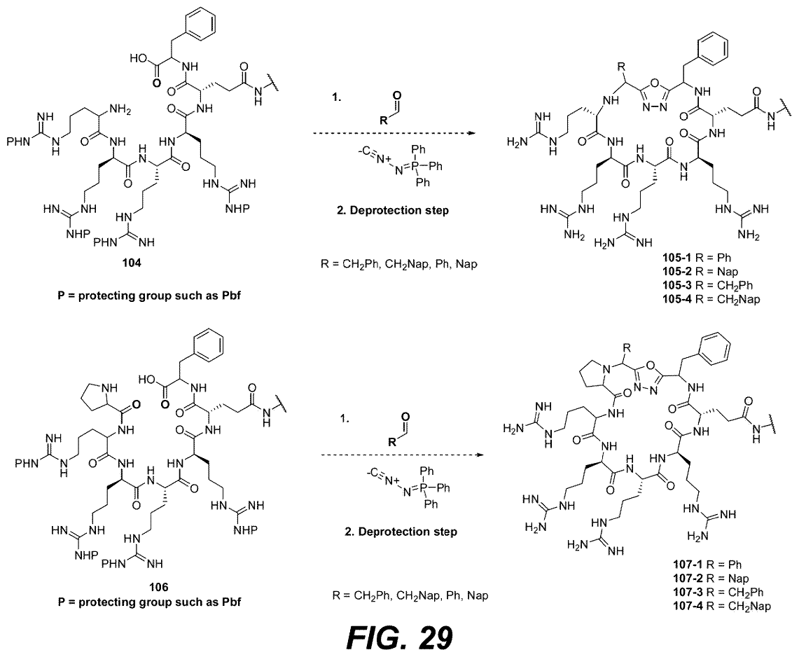

Another example includes novel peptide containing peptide-bond isosteres such as 1,3,4-oxadiazole as illustrated in FIG. 29. In some embodiments, the peptide is constructed by reported methodology (Yudin A. K. et al. Nature Chem. 8, 2016, 1104.)

In some embodiments, the synthesis of the cyclic peptide portion follows general solid-phase synthesis protocols. In some embodiments, the linker between the peptide and the PMO is selected from linkers illustrated in compounds in Examples 15 and 16. In some embodiments, attachment of the peptide to the PMO is achieved as described in Examples 15 and 16 to construct the Peptide-PMO conjugate (e.g. amide bond formation, azide-alkyne click chemistry etc.).

### Example 20: Making Compound 41

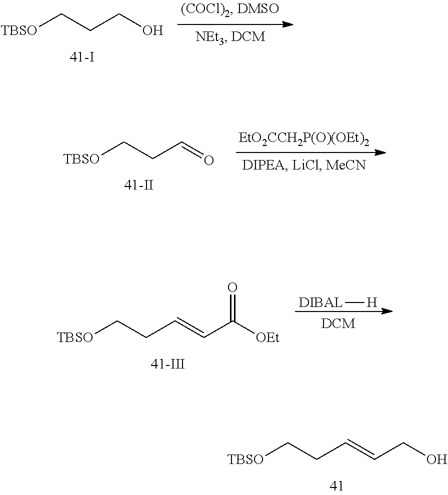

Compound 41: Compound 41 was made by adding DMSO (22.4 ml, 315 mmol) dropwise to a solution of oxalyl chloride (13.8 ml, 158 mmol) in DCM (400 mL) at −78 °C under nitrogen. The solution was stirred at −78 °C for 10 minutes. A solution of 41-(28.1 ml, 131 mmol) in DCM (7.50 mL) was added slowly, and the reaction was stirred at −78 °C for 1 hour. Triethylamine (92 ml, 656 mmol) was then added, and the reaction mixture was maintained at −78 °C for 10 minutes before being warmed to room temperature and stirred for 2 hours. The reaction was then quenched with water, and the phases were separated. The organic layer was washed sequentially with 1 N HCl, NaHCO₃ (sat.), and brine, and was dried over MgSO₄. The solvent was removed via rotary evaporation, producing Compound 41-II (24.9 g, 131 mmol, quant) as a clear oil.

Ethyl 2-(diethoxyphosphoryl)acetate (78.0 ml, 393 mmol) was added to Compound 41-II (24.7 g, 131 mmol) and lithium chloride (16.7 g, 393 mmol) in acetonitrile (525 ml). The solution was then cooled in an ice bath, and N,N-diisopropylethylamine (137 ml, 786 mmol) was added. The reaction was stirred until it was deemed completed by LCMS or TLC (monitoring for disappearance of starting material) and was then worked up with ethyl acetate and aqueous saturated NaHCO₃. The aqueous layer was extracted once more with ethyl acetate, the organics combined, washed with half saturated brine, and dried over Na₂SO₄. The resulting organic layer was concentrated and purified via Biotage purification and afforded Compound 41-III (28.0 g, 108 mmol, 83% yield).

DIBAL-H (1M in hexane, 238 ml, 238 mmol) was added dropwise to a stirred solution of Compound 41-III (28 g, 108 mmol) in dry DCM (310 ml) at −78 °C. After the addition was complete, the solution was warmed to room temperature. Upon complete consumption of the starting material (as indicated by TLC), the reaction mixture was cooled to back to −78 °C and quenched with saturated aqueous Rochelle's salt solution. After stirring at room temperature for 30 minutes, the biphasic mixture was poured into a separatory funnel and the aqueous layer was extracted with ethyl acetate three times. The organic layer was washed with brine, dried with MgSO₄, and concentrated to provide crude Compound 41 (19.3 g, 89 mmol, 82% yield), which was used directly in the next step without further purification. ¹H NMR (400 MHz, CDCl₃) δ ppm 5.60-5.71 (2H, m), 4.05 (2H, br s), 3.60 (2H, t, J=6.75 Hz), 2.18-2.31 (2H, m), 0.84 (9H, s), 0.00 (6H, s); MS (ESI, m/z) Calculated for C₁₁H₂₄O₂Si+H⁺] 217.17. Found 217.1.

### Example 21: Making Compound 42

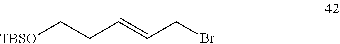 [SMILES: CC(C)(C)[Si](C)(C)OCC/C=C/CBr]

Compound 42: Compound 41 (4.2 g, 19.4 mmol)) in DCM (40 ml) under nitrogen at −40 °C was consecutively treated with DIPEA (6.78 ml, 38.8 mmol) and Ms-Cl (2.269 ml, 29.113 mmol) and the resulting mixture was stirred at −40 °C for one hour. Lithium bromide (16.9 g, 194 mmol) in THF (200 ml) was added and the resulting mixture was stirred at room temperature for one hour, generating a white precipitate. MTBE and saturated NH₄Cl were added. The aqueous layer was separated and extracted with MTBE. The combined organic layers were dried over MgSO₄ and concentrated to give Compound 42 (5.46 g, 19.6 mmol, quant) as a yellowish oil, that was introduced into the next step without further purification. ¹H NMR (400 MHz, CDCl₃) δ ppm 5.69-5.83 (2H, m), 3.95 (2H, d, J=6.25 Hz), 3.65 (2H, t, J=6.57 Hz), 2.28 (2H, q, J=6.17 Hz), 0.89 (9H, s), 0.05 (6H, s); MS (ESI, m/z) Calculated for C₁₁H₂₃BrOSi+H⁺]280.09. Found 280.1.

### Example 22: Compounds Made with General Procedure H

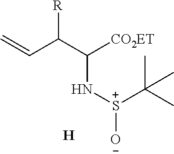

General Procedure H: Allylic bromide (2.0 eq) was added in a single portion to a suspension of indium (3.0 eq) and (R,E) or (S,E) ethyl-2-((tert-butylsulfinyl)imino)acetate (1.0 eq) in DMF (24 vols). The reaction was stirred for 5 hours and was quenched with NH₄Cl (sat). The mixture was filtered through celite and washed with EtOAc. The resulting organic layer washed 3 times with water and once with brine. The organic layer was dried over MgSO₄ and concentrated. The residue was purified using Biotage purification to afford General Compound H as a single diastereomer as a light-yellow oil.

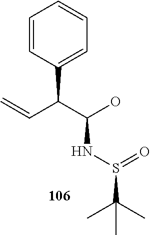 [SMILES: C=C[C@@H](c1ccccc1)[C@@H](O)N[S@@](=O)C(C)(C)C]

Compound 106: 6.4 g, (81% yield) of ethyl (2S,3S)-2-(((S)-tert-butylsulfinyl)amino)-3-phenylpent-4-enoate was prepared from (E)-(3-bromoprop-1-en-1-yl)benzene (7.21 ml, 48.716 mmol) and ethyl (S,E)-2-((tert-butylsulfinyl)imino)acetate (5.00 g, 24.358 mmol) using General Procedure H. ¹H NMR (400 MHz, CDCl₃) δ ppm 7.29-7.35 (2H, m), 7.25 (1H, m, J=6.90 Hz), 7.19 (2H, d, J=7.75 Hz), 6.09 (1H, ddd, J=17.60, 9.00 Hz), 5.13-5.22 (2H, m), 4.13-4.26 (3H, m), 3.92 (1H, br d, J=9.38 Hz), 3.71 (1H, t, J=7.94 Hz), 1.28 (3H, t, J=7.07 Hz), 1.08 (9H, s); MS (ESI, m/z) Calculated for C₁₇H₂₅NO₃S+Na⁺]346.14. Found 346.1.

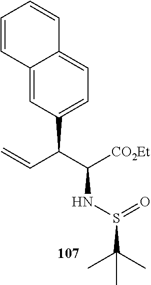 [SMILES: C=C[C@@H](c1ccc2ccccc2c1)[C@H](N[S@@](=O)C(C)(C)C)C(=O)OCC]

Compound 107: 7.26 g, (80% yield) of ethyl (2S,3S)-2-(((S)-tert-butylsulfinyl)amino)-3-(naphthalen-2-yl)pent-4-enoate was prepared from (E)-2-(3-bromoprop-1-en-1-yl)naphthalene (12.04 g, 48.716 mmol) and ethyl (S,E)-2-((tert-butylsulfinyl)imino)acetate (5.00 g, 24.358 mmol) using General Procedure H. ¹H NMR (400 MHz, CHLOROFORM-d) δ ppm 7.74-7.83 (3H, m), 7.63 (1H, s), 7.39-7.53 (2H, m), 7.32 (1H, d, J=8.50 Hz), 6.17 (1H, ddd, J=18.01, 9.38, 9.38 Hz), 5.15-5.26 (2H, m), 4.29 (1H, dd, J=9.07, 7.69 Hz), 4.20 (2H, q, J=7.09 Hz), 3.97 (1H, d, J=9.26 Hz), 3.89 (1H, t, J=7.82 Hz), 1.25 (3H, t, J=6.88 Hz), 1.04 (9H, s); MS (ESI, m/z) Calculated for C₂₁H₂₇NO₃S+H⁺]374.18. Found 374.2.

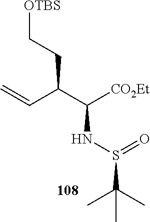 [SMILES: C=C[C@@H](CCO[Si](C)(C)C(C)(C)C)[C@H](N[S@@](=O)C(C)(C)C)C(=O)OCC]

Compound 108: 1.81 g, (92% yield) of ethyl (2S,3R)-3-(2-((tert-butyldimethylsilyl)oxy)ethyl)-2-(((S)-tert-butylsulfinyl)amino)pent-4-enoate was prepared from (E)-((5-bromopent-3-en-1-yl)oxy)(tert-butyl)dimethylsilane (2.72 g, 9.74 mmol) and ethyl ethyl (S,E)-2-((tert-butylsulfinyl)imino)acetate (1.0 g, 4.872 mmol) using General Procedure H. ¹H NMR (400 MHz, CDCl₃) δ ppm 5.58 (1H, ddd, J=16.88, 9.63, 7.75 Hz), 5.06-5.19 (2H, m), 4.22 (2H, q, J=7.13 Hz), 4.12 (1H, br d, J=8.51 Hz), 3.87 (1H, dd, J=8.19, 5.57 Hz), 3.59-3.70 (1H, m), 3.47-3.59 (1H, m), 2.61 (1H, s), 1.75 (1H, br d, J=6.75 Hz), 1.39-1.55 (1H, m), 1.22-1.32 (12H, m), 0.84-0.94 (9H, m), 0.00-0.08 (6H, m); MS (ESI, m/z) Calculated for C₁₉H₃₉NO₄SSi+H⁺]406.25. Found 406.2.

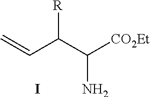

### Example 23: Compounds Made with General Procedure I

General Procedure I: The sulfinyl amino acid (1.0 equiv) was dissolved in THF (10 vols) and then followed by the addition of HCl (4N in dioxanes, 5.0 equiv). The mixture was stirred at room temperature for 40 minutes and was neutralized with saturated NaHCO₃ and diluted with DCM. The mixture was extracted with DCM 3 times, and the organic layer was dried with Na₂SO₄ and concentrated by vacuum to produce General Compound I as a yellow oil. Crude General Compound I was utilized in the next step without additional purification.

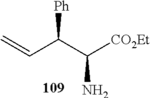

4.3 g (89% yield) of Compound 109 was prepared from Compound 106 (7.1 g, 21.9 mmol) using General Procedure I. ¹H NMR (400 MHz, CDCl₃) δ ppm 7.28-7.38 (2H, m), 7.21-7.26 (3H, m), 6.12 (1H, ddd, J=16.88, 9.13, 8.50 Hz), 5.10-5.22 (2H, m), 4.16 (2H, q, J=7.13 Hz), 3.80 (1H, d, J=7.63 Hz), 3.64 (1H, t, J=7.94 Hz), 1.87 (2H, br s), 1.25 (3H, t, J=7.13 Hz); MS (ESI, m/z) Calculated for [C13H17NO2+H⁺]220.14. Found 220.1.

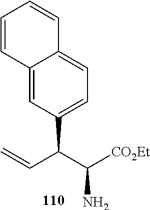 [SMILES: C=C[C@@H](c1ccc2ccccc2c1)[C@H](N)C(=O)OCC]

5.9 g (100% yield) of Compound 110 was prepared from Compound 107 (8.2 g, 21.4 mmol) using General Procedure I. ¹H NMR (400 MHz, CDCl₃) δ ppm 7.82 (3H, br t, J=8.57 Hz), 7.69 (1H, s), 7.41-7.54 (2H, m), 7.37 (1H, d, J=8.38 Hz), 6.21 (1H, ddd, J=17.01, 9.13, 8.50 Hz), 5.05-5.27 (2H, m), 4.18 (2H, q, J=7.00 Hz), 3.88 (1H, d, J=7.63 Hz), 3.78 (1H, dd, J=8.13, 7.38 Hz), 1.50 (2H, br s), 1.25 (3H, t, J=7.00 Hz); MS (ESI, m/z) Calculated for C₁₇H₁₉NO₂+H⁺] 270.15. Found 270.2.

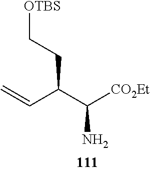 [SMILES: C=C[C@@H](CCO[Si](C)(C)C(C)(C)C)[C@H](N)C(=O)OCC]

1.5 g (92% yield) of Compound 111 was prepared from Compound 108 (2.9 g, 7.1 mmol) using General Procedure I. ¹H NMR (400 MHz, CDCl₃) δ ppm 5.73 (1H, dt, J=16.95, 9.72 Hz), 5.14-5.34 (2H, m), 4.28 (2H, q, J=7.00 Hz), 3.70-3.93 (1H, m), 3.60-3.69 (1H, m), 3.58 (1H, d, J=4.88 Hz), 2.58-2.71 (1H, m), 1.74-1.99 (1H, m), 1.59-1.70 (1H, m), 0.99 (9H, s), 0.13 (6H, s); MS (ESI, m/z) Calculated for C₁₅H₃₁NO₃Si+H⁺] 302.22. Found 302.3.

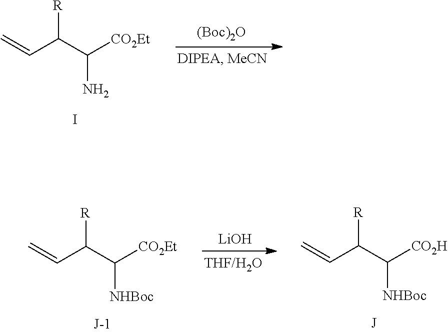

### Example 24: Compounds Made with General Procedure J

General Procedure J: Di-tert-butyl dicarbonate (1.1 equiv) was added to General Compound 1 (1.0 equiv) dissolved in MeCN (10 vols) followed by DIPEA (1.2 equiv). The resulting suspension was stirred at ambient temperature for 1 hour before being diluted with CH₂Cl₂, washed with H₂O, 10% citric acid solution, and saturated NaHCO₃, then dried over anhydrous Na₂SO₄. The resulting suspension was filtrated, and the organics were evaporated. The resulting compound was purified via Biotage purification to furnish General Compound J-4.

General Compound J-4 (1.0 equiv) was dissolved in THF (8 vols) and water (2 vols). Lithium hydroxide, monohydrate (5.0 equiv) was added in a single portion at rt. The reaction was stirred vigorously until completion via LCMS. The solution was concentrated and partitioned between water and EtOAc. The aqueous phase's pH was adjusted to the pH-1 and extracted 3 times with EtOAc. The combined organics were washed with brine, dried over Na₂SO₄ and concentrated to afford General Compound J. The isolated material was of sufficient purity to proceed to the next step.

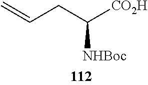

Compound 112 was synthesized using the procedure from Buschauer, A., J. Med. Chem. 2016, 59, 13, 6045. ¹H NMR (400 MHz, CDCl₃) δ ppm 5.67-5.82 (1H, m), 5.14-5.22 (2H, m), 5.02 (1H, br d, J=7.25 Hz), 4.40 (1H, br d, J=5.75 Hz), 2.44-2.68 (2H, m), 1.46 (9H, s); MS (ESI, m/z) Calculated for [C₁₀H₁₇NO₄+Na⁺] 238.10. Found 238.1.

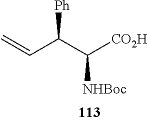 [SMILES: C=C[C@@H](c1ccccc1)[C@H](NC(=O)OC(C)(C)C)C(=O)O]

2.5 g (86% yield) of Compound 113 was prepared from Compound 109 (2.5 g, 17.3 mmol) using General Procedure J. ¹H NMR (400 MHz, CDCl₃) δ ppm 7.28-7.37 (3H, m), 7.21-7.26 (2H, m), 6.11 (1H, ddt, J=17.50, 8.80, 8.80, 8.80 Hz), 5.23 (2H, br d, J=13.51 Hz), 4.81 (1H, br d, J=8.38 Hz), 4.68 (1H, br t, J=6.88 Hz), 3.76-3.95 (1H, m), 1.40 (9H, s); MS (ESI, m/z) Calculated for [C₁₆H₂₁NO₄+Na⁺] 314.14. Found 314.1.

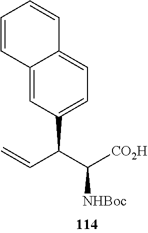 [SMILES: C=C[C@@H](c1ccc2ccccc2c1)[C@H](NC(=O)OC(C)(C)C)C(=O)O]

3.1 g (77% yield) of Compound 114 was prepared from Compound 110 (3.0 g, 11.1 mmol) using General Procedure J. ¹H NMR (400 MHz, CDCl₃) δ ppm 7.83 (3H, br d, J=8.25 Hz), 7.68 (1H, s), 7.45-7.53 (2H, m), 7.37 (1H, br d, J=8.38 Hz), 6.20 (1H, ddt, J=17.50, 9.00, 9.00, 9.00 Hz), 5.22-5.31 (2H, m), 4.86 (1H, br d, J=7.75 Hz), 4.56-4.80 (1H, m), 4.06 (1H, br s), 1.37 (9H, s); MS (ESI, m/z) Calculated for [C₂₀H₂₃NO₄+Na⁺]364.1. Found 364.0.

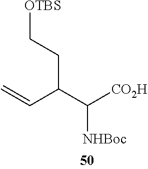 [SMILES: C=CC(CCO[Si](C)(C)C(C)(C)C)C(NC(=O)OC(C)(C)C)C(=O)O]

Compound 50 is prepared from Compound 111 using General Procedure J.

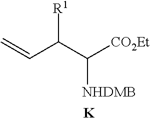

### Example 25: Compounds Made with General Procedure K

General Procedure K: The free amino ester (1.0 equiv), an equimolar amount of 2,4-dimethoxybenzaldehyde (1.0 equiv) and acetic acid (2.0 equiv) were dissolved in anhydrous EtOH (33 vols). Sodium cyanoborohydride (2 equiv) was added in a single portion and the reaction was permitted to proceed for 12 hours at ambient temperature. The excess borohydride was quenched with 10% Na₂CO₃ in water followed by the addition of DCM. The aqueous layer was extracted 3 times with DCM and dried with anhydrous MgSO₄. The organic layer was removed under reduced pressure to provide a clear yellow oil. The desired compound was purified using Biotage purification yielding the corresponding reductive amination product.

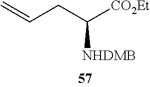

Compound 57 was synthesized using the procedure from Creighton, C. J., Bioorg. Med. Chem. 12, 2004, 4375. ¹H NMR (400 MHz, CHLOROFORM-d) δ ppm 7.12 (1H, d, J=7.88 Hz), 6.41-6.46 (2H, m), 5.67-5.79 (1H, m), 5.03-5.14 (2H, m), 3.80 (6H, s), 3.71-3.77 (1H, m), 3.62-3.71 (4H, m), 3.35 (1H, t, J=6.57 Hz), 2.42 (2H, br t, J=6.88 Hz), 1.20-1.35 (1H, m); MS (ESI, m/z) Calculated for [C15H21NO4+Na⁺]302.14. Found 302.1.

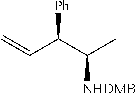

Compound 115 is prepared from Compound 109 using General Procedure K.

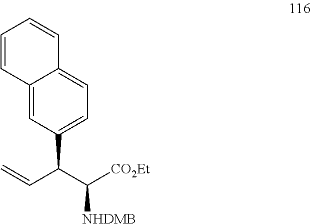

670 mg (86% yield) of Compound 116 was prepared from Compound 110 (500 mg, 1.86 mmol) using General Procedure K. ¹H NMR (400 MHz, CHLOROFORM-d) δ ppm 7.71-7.83 (3H, m), 7.54 (1H, s), 7.42-7.50 (2H, m), 7.23 (1H, br d, J=8.38 Hz), 6.95-7.06 (1H, m), 6.34-6.40 (1H, m), 6.20 (1H, s), 5.99-6.10 (1H, m), 4.97-5.16 (2H, m), 4.12-4.24 (2H, m), 3.80 (3H, s), 3.71-3.77 (2H, m), 3.58-3.71 (2H, m), 3.44 (1H, d, J=13.63 Hz), 3.17 (3H, s), 0.80-0.95 (4H, m); MS (ESI, m/z) Calculated for [C25H27NO4+H⁺] 420.22. Found 420.2.

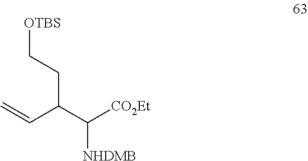

Compound 63 is prepared from Compound 111 using General Procedure K.

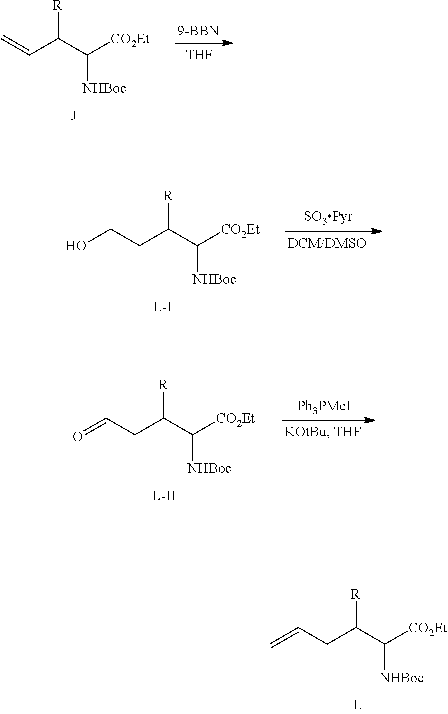

### Example 26: Compounds Made with General Procedure L

General Procedure L: General Compound J (1.0 equiv) is dissolved in THF (5 vols) and cooled at −78 °C. 9-BBN (2.5 equiv) is added dropwise to the reaction mixture. The reaction mixture is stirred at this temperature for 1 hour, then allowed to warm to room temperature and stirred for additional 2 hours. Then the reaction is cooled at 0 °C and sodium acetate (5.0 equiv) solution in water (12 vols) mixed with hydrogen peroxide (30% wt/wt, 5.0 equiv) is added dropwise into the reaction mixture. The reaction is then allowed to warm to room temperature and stirred overnight. The aqueous layer is extracted with EtOAc 3 times and dried over anhydrous Na₂SO₄. After filtration all the solvent is removed under reduced pressure. The crude reaction mixture is purified by Biotage purification to afford General Compound L-I.

General Compound L-I (1.0 equiv) and triethylamine (4.0 equiv) in dichloromethane (10 vols) at 0 °C is reacted with sulfur trioxide pyridine complex (3.0 equiv) in dimethyl sulfoxide (3 vols) by dropwise addition followed by reaction for an additional 3 hours. The reaction is quenched with water and diluted with ethyl acetate. The organic layer is washed with water, and brine, dried over MgSO₄ filtered, and concentrated in vacuo to provide General Compound L-II.

A flask is flame dried, then cooled to −78 °C. Methyltriphenylphosphonium iodide (2.2 equiv) and potassium tert-butoxide (2.2 equiv) are added to the flask and dissolved in dry THF (10 vols). This solution is then allowed to warm up to 0 °C over 1.5 hours. General Compound L-II (1.0 equiv) is dissolved in THF (10 vols) in a flask cooled to 0 °C. This solution is cannulated into the solution containing the phosphonium ylide and stirred at 0 °C for 30 minutes. The reaction mixture is quenched with saturated NH₄Cl and vigorously stirred for 5 minutes. The reaction is then diluted with water and extracted with EtOAc three times. Pooled organic extracts are dried over Na₂SO₄, filtered and concentrated in vacuo. The crude product is purified via Biotage purification to give General Compound L.

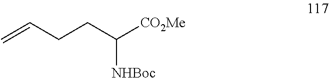 [SMILES: C=CCCC(NC(=O)OC(C)(C)C)C(=O)OC]

Compound 117 is prepared from the Methyl ester of Compound 112 using General Procedure L.

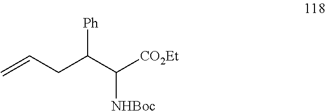 [SMILES: C=CCC(c1ccccc1)C(NC(=O)OC(C)(C)C)C(=O)OCC]

Compound 118 is prepared from the Ethyl ester of Compound 113 using General Procedure L.

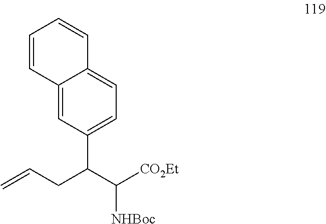 [SMILES: C=CCC(c1ccc2ccccc2c1)C(NC(=O)OC(C)(C)C)C(=O)OCC]

Compound 119 is prepared from the Ethyl ester of Compound 114 using General Procedure L.

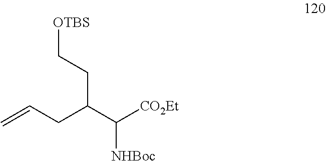 [SMILES: C=CCC(CCO[Si](C)(C)C(C)(C)C)C(NC(=O)OC(C)(C)C)C(=O)OCC]

Compound 120 is prepared from the Ethyl ester of Compound 115 using General Procedure L.

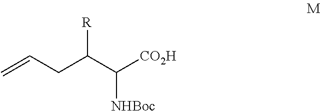

### Example 27: Compounds Made with General Procedure M

General Procedure M: General Compound L (1.0 equiv) is dissolved in THF (8 vols) and water (2 vols). Lithium hydroxide, monohydrate (5.0 equiv) is added in a single portion at room temperature. The reaction is stirred vigorously until completion of the reaction as measured by LCMS. The solution is concentrated and partitioned between water and EtOAc. The aqueous phase's pH is adjusted to the pH-1 and extracted 3 times with EtOAc. The combined organics are washed with brine, dried over Na₂SO₄, and concentrated to afford General Compound M. The isolated material is of sufficient purity to proceed to the next step.

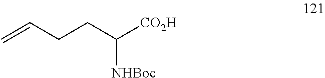 [SMILES: C=CCCC(NC(=O)OC(C)(C)C)C(=O)O]

Compound 121 is prepared from Compound 117 using General Procedure M.

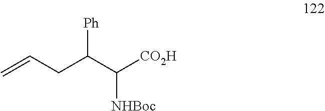 [SMILES: C=CCC(c1ccccc1)C(NC(=O)OC(C)(C)C)C(=O)O]

Compound 122 is prepared from Compound 118 using General Procedure M.

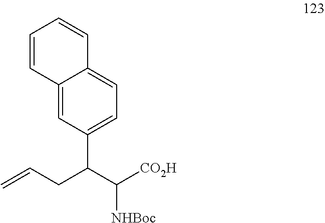 [SMILES: C=CCC(c1ccc2ccccc2c1)C(NC(=O)OC(C)(C)C)C(=O)O]

Compound 123 is prepared from Compound 119 using General Procedure M.

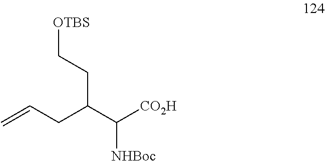 [SMILES: C=CCC(CCO[Si](C)(C)C(C)(C)C)C(NC(=O)OC(C)(C)C)C(=O)O]

Compound 124 is prepared from Compound 120 using General Procedure M.

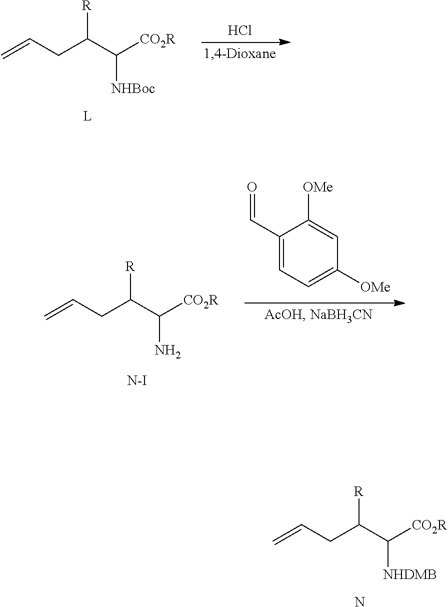

### Example 28: Compounds Made with General Procedure N

General Procedure N: General Compound L (1.0 equiv) is dissolved in 1,4-dioxane (4 vols), 4N hydrochloric acid/1,4-dioxane solution (13 equiv) is added and the resulting mixture was stirred at room temperature for 2 hours. The solvent is evaporated under reduced pressure to give General Compound N-I.

General Compound N-I (1.0 equiv), an equimolar amount of 2,4-dimethoxybenzaldehyde (1 equiv) and acetic acid (2 equiv) are dissolved in anhydrous EtOH (33 vols). Sodium cyanoborohydride (2 equiv) is added in a single portion and the reaction is permitted to proceed for 12 hours at ambient temperature. The excess borohydride is quenched with 10% Na₂CO₃ in water followed by the addition of DCM. The aqueous layer is extracted 3 times with DCM and dried with anhydrous MgSO₄. The organic layer is removed under reduced pressure to provide a clear yellow oil. The desired compound is purified using Biotage purification yielding General Compound N.

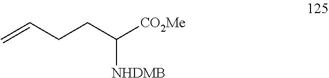

Compound 125 is prepared from Compound 117 using General Procedure N.

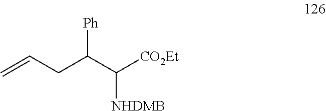

Compound 126 is prepared from Compound 118 using General Procedure N.

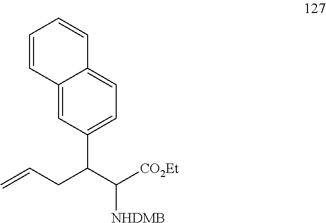

Compound 127 is prepared from Compound 119 using General Procedure N.

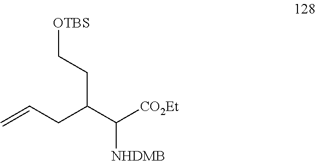

Compound 128 is prepared from Compound 120 using General Procedure N.

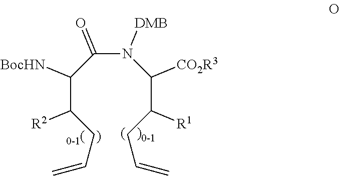

### Example 29: Compounds Made with General Procedure O

General Procedure O: General Compound J or General Compound M (1.0 equiv) is dissolved in DMA (10 vols) and is followed by the addition of N-Methylmorpholine(4-) (10 eq) and HATU (1.2 equiv) in turn at 0 °C. The mixture is stirred at 0° for 30 minutes, General Compound K or General Compound N (2.0 equiv) dissolved in DMA (10 vols) is added dropwise. The solution is stirred at room temperature overnight. The reaction solution is poured into water and extracted with ethyl acetate. The combined organic layers are washed with 1 N HCl, aqueous NaHCO₃, and brine, then dried over Na₂SO₄, and concentrated to give a crude oil, which is purified by biotage purification, providing General Compound 0 as a mixture of rotamers

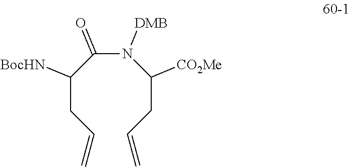

Compound 60-1 is prepared from Compound 112 and Compound 57 using General Procedure O.

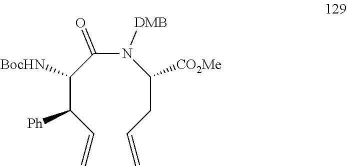

Compound 129 is prepared from Compound 109 and Compound 57 using General Procedure O.

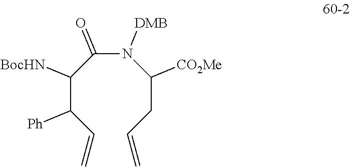

Compound 60-2 is prepared from Compound 52 and Compound 57 using General Procedure O.

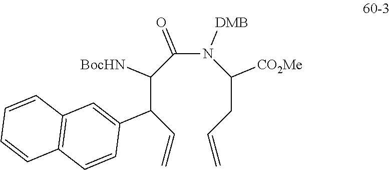

Compound 60-3 is prepared from Compound 51 and Compound 57 using General Procedure O.

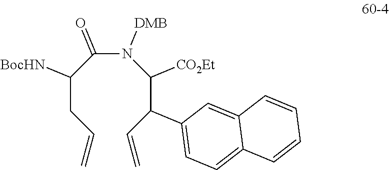

Compound 60-4 is prepared from Compound 112 and Compound 51 using General Procedure O.

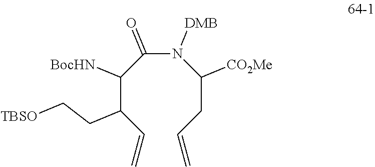

Compound 64-1 is prepared from Compound 50 and Compound 57 using General Procedure O.

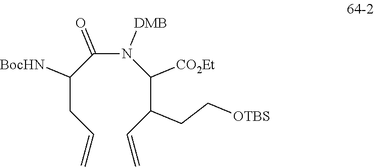

Compound 64-2 is prepared from Compound 112 and Compound 63 using General Procedure O.

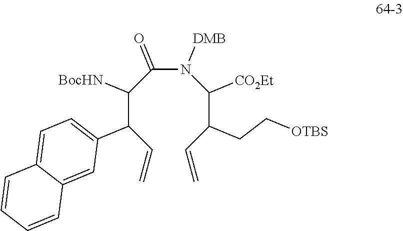

Compound 64-3 is prepared from Compound 51 and Compound 63 using General Procedure O.

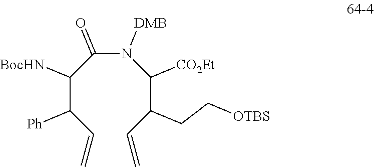

Compound 64-4 is prepared from Compound 52 and Compound 63 using General Procedure O.

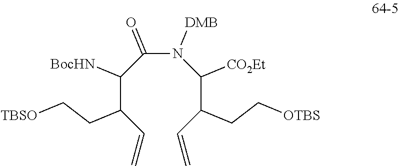

Compound 64-5 is prepared from Compound 50 and Compound 63 using General Procedure O.

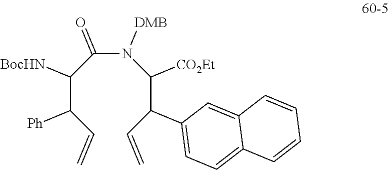

Compound 60-5 is prepared from Compound 52 and Compound 58 using General Procedure O.

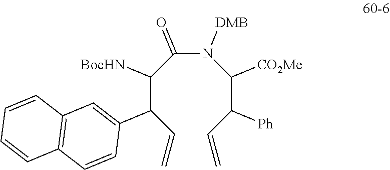

Compound 60-6 is prepared from Compound 51 and Compound 59 using General Procedure O.

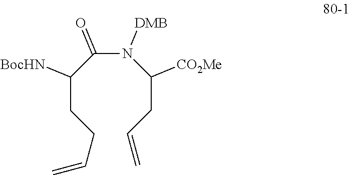

Compound 80-1 is prepared from Compound 121 and Compound 57 using General Procedure O.

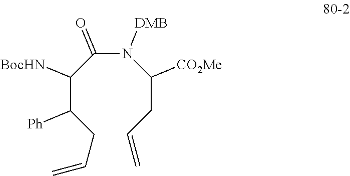

Compound 80-2 is prepared from Compound 122 and Compound 57 using General Procedure O.

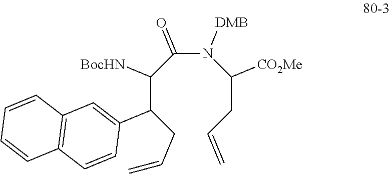

Compound 80-3 is prepared from Compound 123 and Compound 57 using General Procedure O.

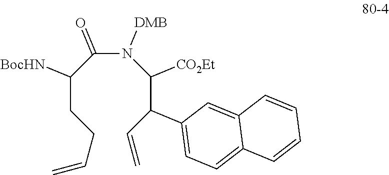

Compound 80-4 is prepared from Compound 121 and Compound 58 using General Procedure O.

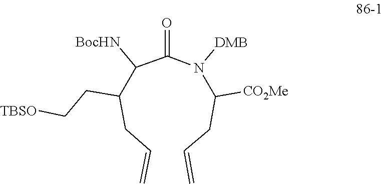

Compound 86-1 is prepared from Compound 124 and Compound 57 using General Procedure O.

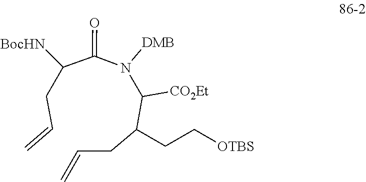

Compound 86-2 is prepared from Compound 112 and Compound 128 using General Procedure O.

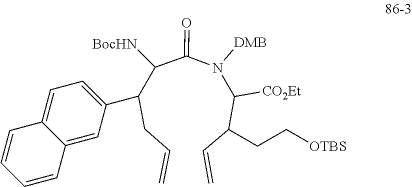

Compound 86-3 is prepared from Compound 123 and Compound 63 using General Procedure O.

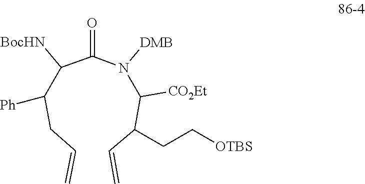

Compound 86-4 is prepared from Compound 122 and Compound 63 using General Procedure O.

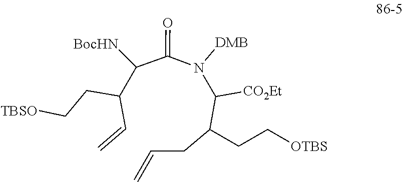

Compound 86-5 is prepared from Compound 50 and Compound 128 using General Procedure O.

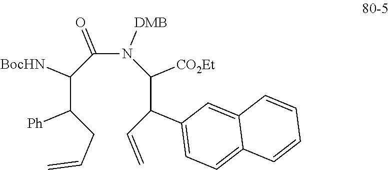

Compound 80-5 is prepared from Compound 122 and Compound 51 using General Procedure O.

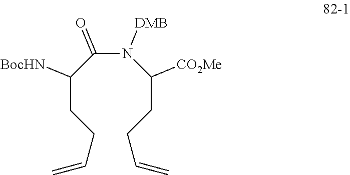

Compound 82-1 is prepared from Compound 121 and Compound 125 using General Procedure O.

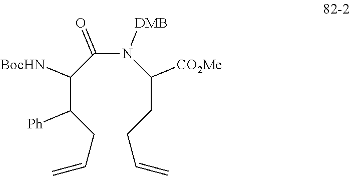

Compound 82-2 is prepared from Compound 122 and Compound 125 using General Procedure O.

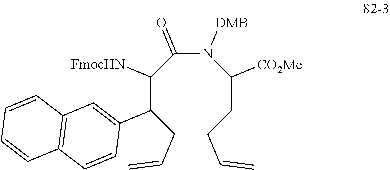

Compound 82-3 is prepared from Compound 123 and Compound 125 using General Procedure O.

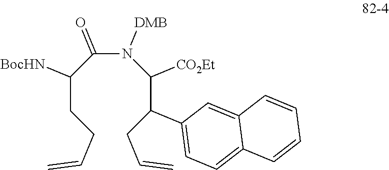

Compound 82-4 is prepared from Compound 121 and Compound 127 using General Procedure O.

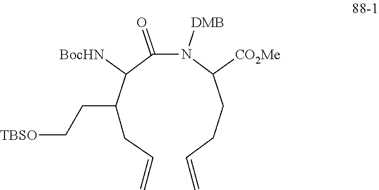

Compound 88-1 is prepared from Compound 124 and Compound 125 using General Procedure O.

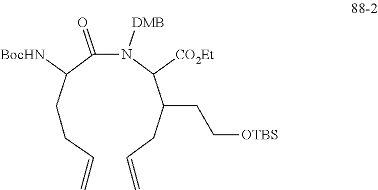

Compound 88-2 is prepared from Compound 121 and Compound 128 using General Procedure O.

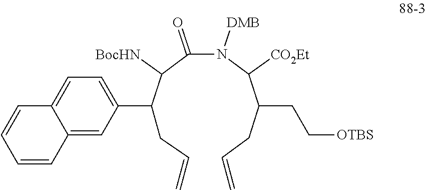

Compound 88-3 is prepared from Compound 123 and Compound 128 using General Procedure O.

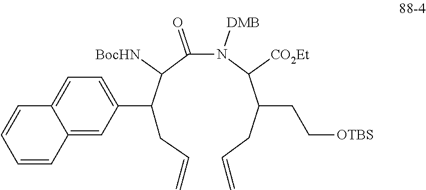

Compound 88-4 is prepared from Compound 123 and Compound 128 using General Procedure O.

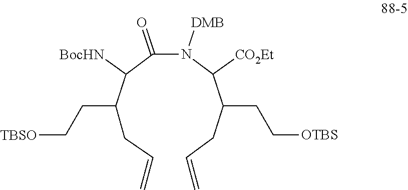

Compound 88-5 is prepared from Compound 124 and Compound 128 using General Procedure O.

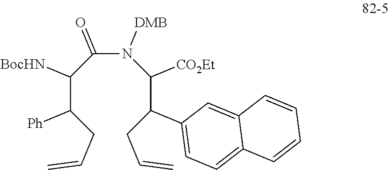

Compound 82-5 is prepared from Compound 122 and Compound 127 using General Procedure O.

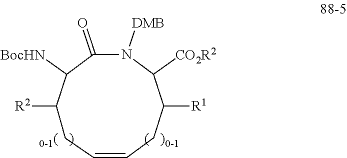

### Example 30: Compounds Made with General Procedure P

General Procedure P: General Compound O (1.0 equiv) is dissolved in DCE (750 vol) and is deoxygenated by sparging with N2for 30 minutes. Grubbs I catalyst (20 mol %) is added to the solution and the resulting solution is refluxed for 60 hours. After completion, the solvent is removed, and the crude product is purified by Biotage purification, providing General Compound P.

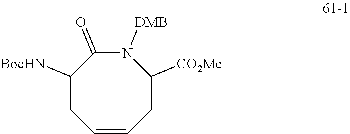

Compound 61-1 is prepared from Compound 60-1 using General Procedure P.

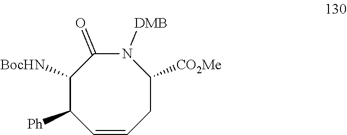

Compound 130 is prepared from Compound 129 using General Procedure P.

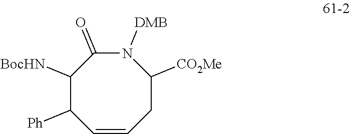

Compound 61-2 is prepared from Compound 60-2 using General Procedure P.

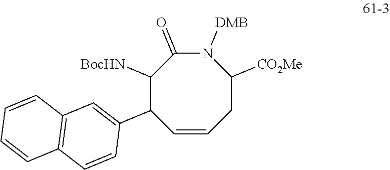

Compound 61-3 is prepared from Compound 60-3 using General Procedure P.

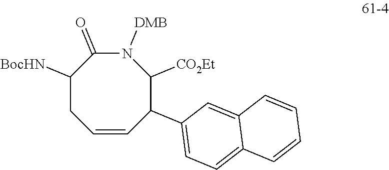

Compound 61-4 is prepared from Compound 60-4 using General Procedure P.

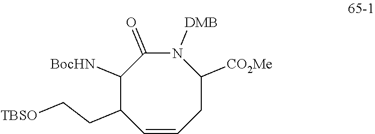

Compound 65-1 is prepared from Compound 64-1 using General Procedure P.

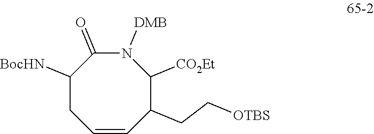

Compound 65-2 is prepared from Compound 64-2 using General Procedure P.

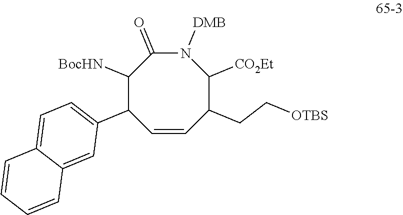

Compound 65-3 is prepared from Compound 64-3 using General Procedure P.

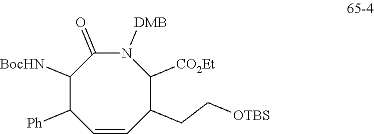

Compound 65-4 is prepared from Compound 64-4 using General Procedure P.

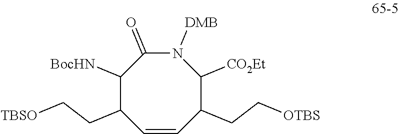

Compound 65-5 is prepared from Compound 64-5 using General Procedure P.

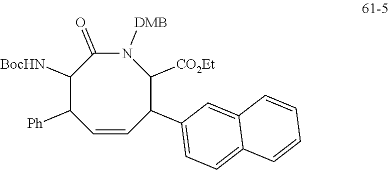

Compound 61-5 is prepared from Compound 60-5 using General Procedure P.

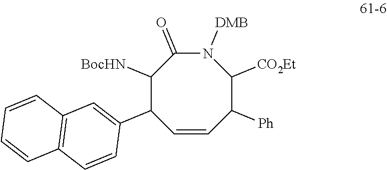

Compound 61-6 is prepared from Compound 60-6 using General Procedure P.

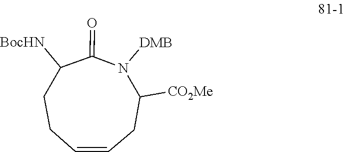

Compound 81-1 is prepared from Compound 80-1 using General Procedure P.

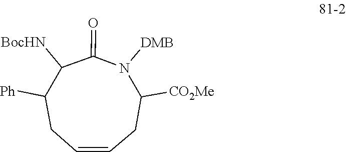

Compound 81-2 is prepared from Compound 80-2 using General Procedure P.

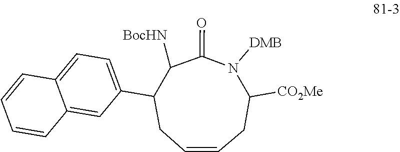

Compound 81-3 is prepared from Compound 80-3 using General Procedure P.

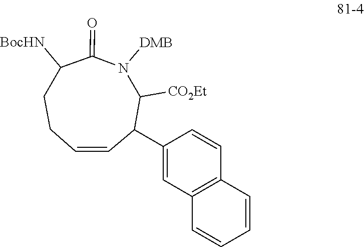

Compound 81-4 is prepared from Compound 80-4 using General Procedure P.

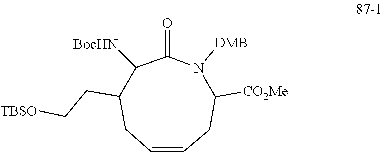

Compound 87-1 is prepared from Compound 86-1 using General Procedure P.

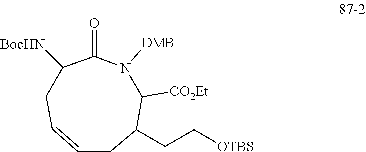

Compound 87-2 is prepared from Compound 86-2 using General Procedure P.

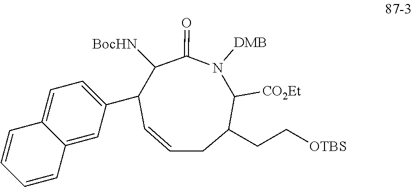

Compound 87-3 is prepared from Compound 86-3 using General Procedure P.

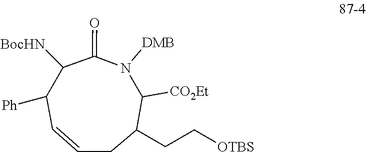

Compound 87-4 is prepared from Compound 86-4 using General Procedure P.

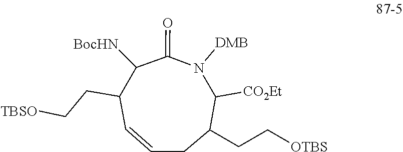

Compound 87-5 is prepared from Compound 86-5 using General Procedure P.

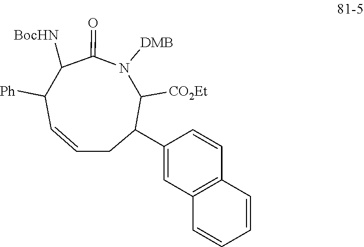

Compound 81-5 is prepared from Compound 80-5 using General Procedure P.

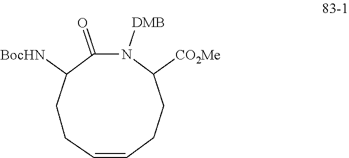

Compound 83-1 is prepared from Compound 82-1 using General Procedure P.

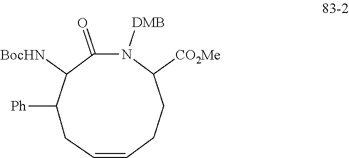

Compound 83-2 is prepared from Compound 82-2 using General Procedure P.

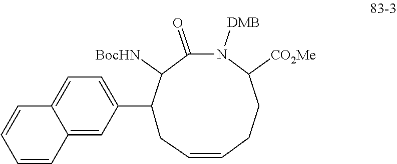

Compound 83-3 is prepared from Compound 82-3 using General Procedure P.

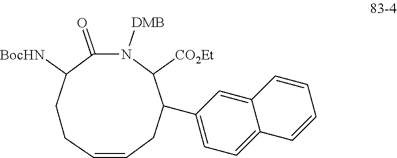

Compound 83-4 is prepared from Compound 82-4 using General Procedure P.

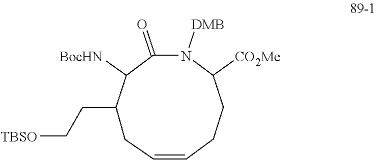

Compound 89-1 is prepared from Compound 88-1 using General Procedure P.

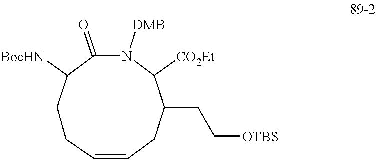

Compound 89-2 is prepared from Compound 88-2 using General Procedure P.

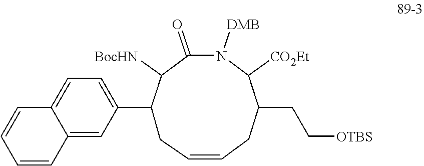

Compound 89-3 is prepared from Compound 88-3 using General Procedure P.

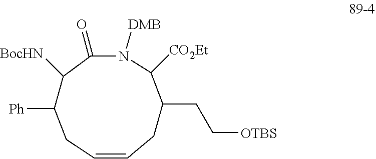

Compound 89-4 is prepared from Compound 88-4 using General Procedure P.

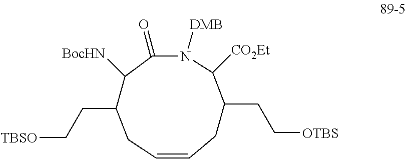

Compound 89-5 is prepared from Compound 88-5 using General Procedure P.

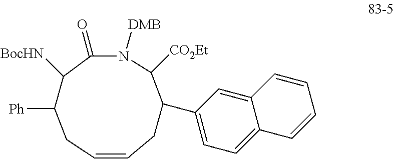

Compound 83-5 is prepared from Compound 82-5 using General Procedure P.

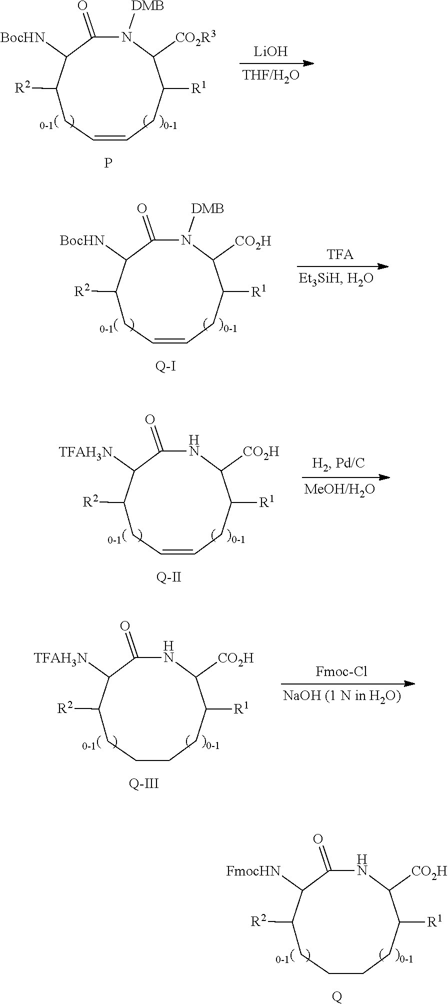

### Example 31: Compounds Made with General Procedure Q

General Procedure Q: General Compound P (1.0 equiv) is dissolved in THF (8 vols) and water (2 vols). Lithium hydroxide monohydrate (5.0 equiv) is added in a single portion at room temperature and is stirred overnight. The solution is concentrated and partitioned between water and EtOAc. The aqueous phase's pH is adjusted to the pH~1 and extracted 3 times with EtOAc. The combined organics are washed with brine, dried over Na₂SO₄, and concentrated, affording General Compound Q-4. The isolated material is of sufficient purity to proceed to the next step.

General Compound Q-4 (1.0 equiv) is treated with a solution of triethyl silane (0.4 vols) and water (0.2 vols) in TFA (5 vols). The reaction is allowed to proceed for 16 hours, after which the solvent is removed, and the crude oil is treated with excess ethyl ether. General Compound Q-II is triturated in ether for 4 hours and is collected on a glass fritted funnel, which is of sufficient purity to proceed to the next step.

General Compound Q-II (1 equiv) is dissolved in a mixture of methanol (75 vols) and water (25 vols). To this solution was added approximately of 10% Pd/C (25% weight/weight) and the reaction mixture is treated with 1 atmosphere of hydrogen and allowed to react for 4 hours at room temperature. After 4 hours, the reaction mixture is filtered over a pad of Celite and washed 3 times with methanol. The organics are combined, and the solvent is removed under reduced pressure to yield the General Compound Q-III.

Fmoc-Cl (1.2 equivalents) is added to a solution of General Compound Q-III (1.0 equiv) in 1 N NaOH (20 vols) at 0 °C. After 24 hours at room temperature, the reaction is diluted the reaction mixture with H₂O and adjust the pH 2-3 with 2 N HCl. The aqueous layer is extracted three times with EtOAc. The combined organic phases are washed with brine. After drying over Na₂SO₄ and filtration, the solvent is removed by rotary evaporation to afford General Compound Q.

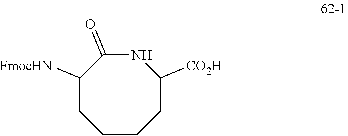

Compound 62-1 is prepared from Compound 61-1 using General Procedure Q.

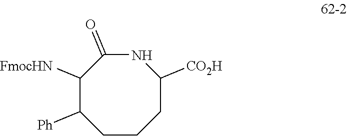

Compound 62-2 is prepared from Compound 61-2 using General Procedure Q.

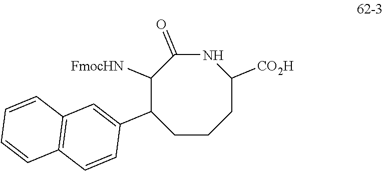

Compound 62-3 is prepared from Compound 61-3 using General Procedure Q.

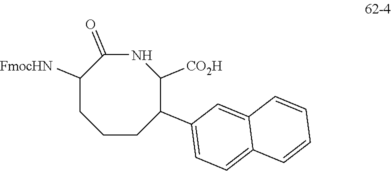

Compound 62-4 is prepared from Compound 61-4 using General Procedure Q.

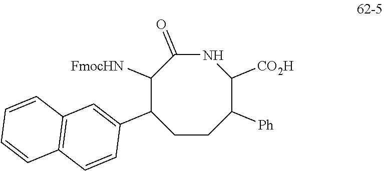

Compound 62-5 is prepared from Compound 61-5 using General Procedure Q.

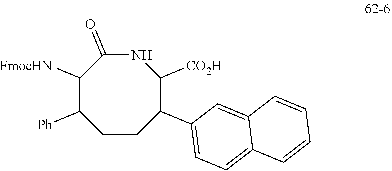

Compound 62-6 is prepared from Compound 61-6 using General Procedure Q.

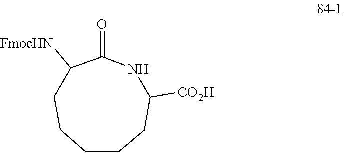

Compound 84-1 is prepared from Compound 81-1 using General Procedure Q.

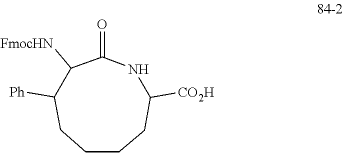

Compound 84-2 is prepared from Compound 91-2 using General Procedure Q.

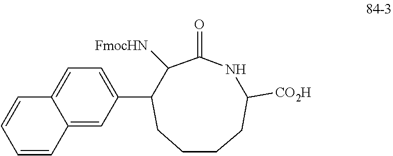

Compound 84-3 is prepared from Compound 81-3 using General Procedure Q.

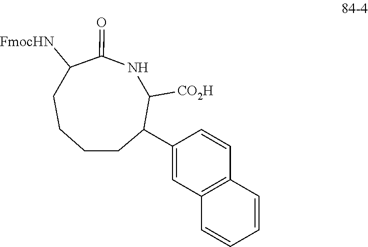

Compound 84-4 is prepared from Compound 81-4 using General Procedure Q.

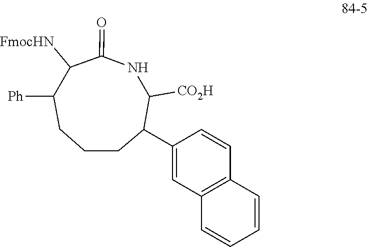

Compound 84-5 is prepared from Compound 81-5 using General Procedure Q.

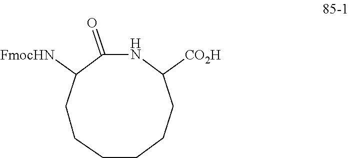

Compound 85-1 is prepared from Compound 83-1 using General Procedure Q.

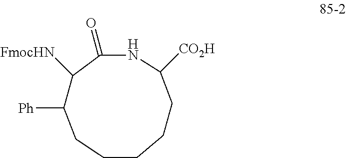

Compound 85-2 is prepared from Compound 83-2 using General Procedure Q.

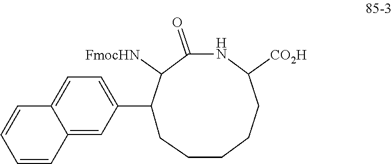

Compound 85-3 is prepared from Compound 83-3 using General Procedure Q.

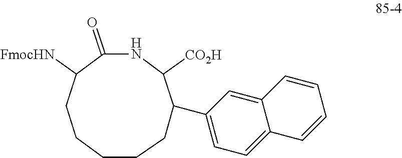

Compound 85-4 is prepared from Compound 83-4 using General Procedure Q.

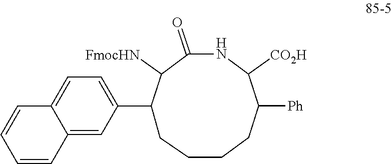

Compound 85-5 is prepared from Compound 83-5 using General Procedure Q.

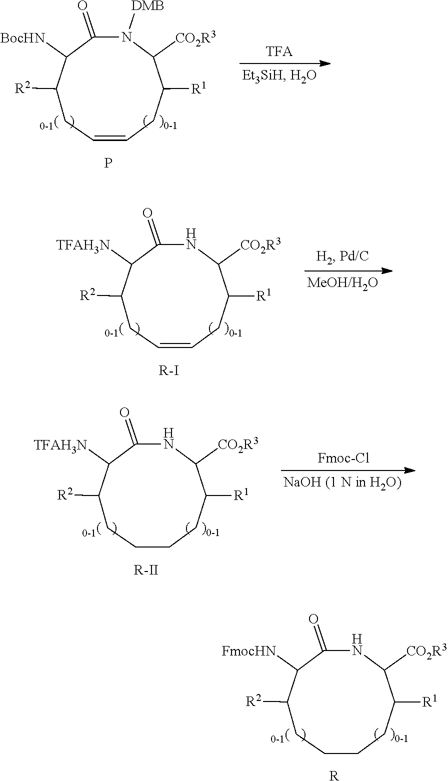

### Example 32: Compounds Made with General Procedure R

General Procedure R: General Compound P (1.0 equiv) is treated with a solution of triethyl silane (0.4 vols) and water (0.2 vols) in TFA (5 vols). The reaction is allowed to proceed for 16 hours after which the solvent is removed, and the crude oil is treated with excess ethyl ether. General Compound R-I is triturated in ether for 4 hours and is collected on a glass fritted funnel, which is of sufficient purity to proceed to the next step.

General Compound R-I (1.0 equiv) is dissolved in a mixture of methanol (75 vols) and water (25 vols). To this solution is added approximately of 10% Pd/C (25% weight/weight) and the reaction mixture is treated with 1 atmosphere of hydrogen and allowed to react for 4 hours at room temperature. After 4 hours, the reaction mixture is filtered over a pad of Celite and washed 3 times with methanol. The organics are combined, and the solvent is removed under reduced pressure to yield General Compound R-II.

N, N-diisopropylethylamine (3.0 equiv) and Fmoc-Cl (1.2 equivalents) is added to a solution of General Compound R-II (1.0 equiv) in dichloromethane (10 vols) at 0 °C. The reaction is stirred at room temperature for 66 hours. The mixture is concentrated and taken up in diethyl ether and washed with 10% aqueous citric acid and saturated aqueous sodium bicarbonate. The organic layer is dried over magnesium sulfate, filtered, and concentrated. General Compound R is purified via Biotage purification.

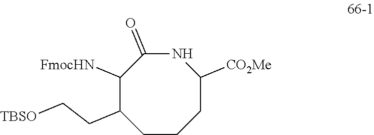

Compound 66-1 is prepared from Compound 65-1 using General Procedure R.

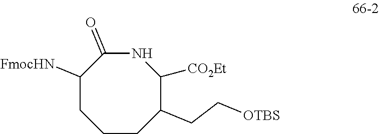

Compound 66-2 is prepared from Compound 65-2 using General Procedure R.

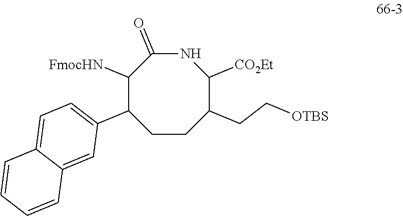

Compound 66-3 is prepared from Compound 65-3 using General Procedure R.

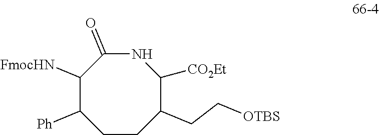

Compound 66-4 is prepared from Compound 65-4 using General Procedure R.

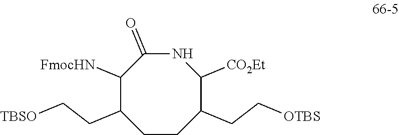

Compound 66-5 is prepared from Compound 65-5 using General Procedure R.

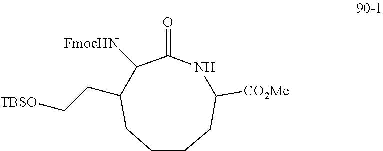

Compound 90-1 is prepared from Compound 87-1 using General Procedure R.

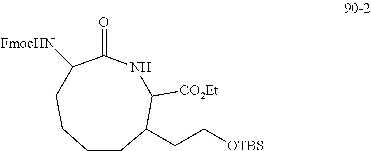

Compound 90-2 is prepared from Compound 90-2 using General Procedure R.

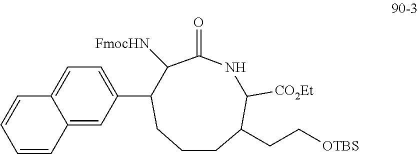

Compound 90-3 is prepared from Compound 90-3 using General Procedure R.

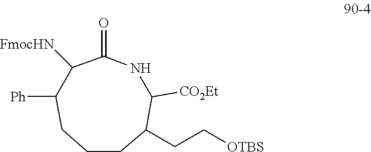

Compound 90-4 is prepared from Compound 87-4 using General Procedure R

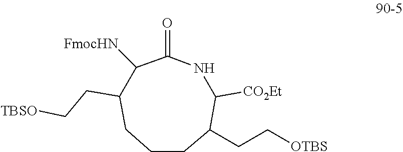

Compound 90-5 is prepared from Compound 87-5 using General Procedure R.

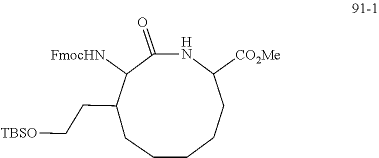

Compound 91-1 is prepared from Compound 89-1 using General Procedure R.

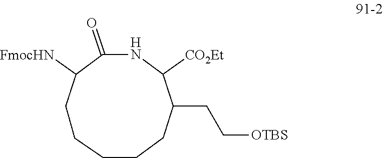

Compound 91-2 is prepared from 89-2 using General Procedure R.

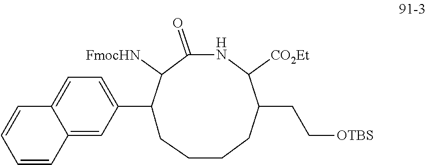

Compound 91-3 is prepared from Compound 89-3 using General Procedure R.

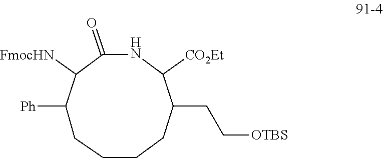

Compound 91-4 is prepared from Compound 89-4 using General Procedure R.

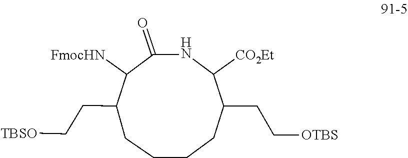

Compound 91-5 is prepared from Compound 89-5 using General Procedure R.

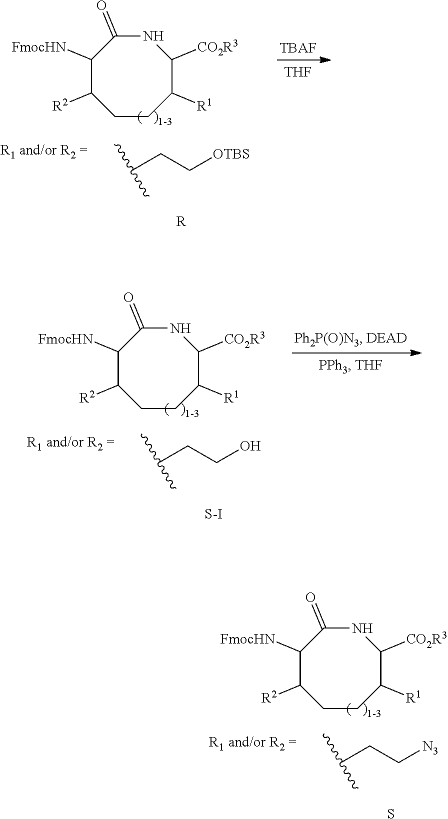

### Example 33: Compounds Made with General Procedure S

General Procedure S: To a solution of General Compound R (1.0 equiv) in THF (20 vols) at 0 °C is added tetrabutyl ammonium fluoride 1 M in THF (2.0 equiv per alcohol). The obtained mixture is stirred at 0 °C for 1 minute, before being concentrated in vacuo. The obtained crude product is purified by Biotage purification to the General Compound S-4.

A solution of General Compound S-4 (1.0 equiv) in THF (20 vols) is cooled to 0 °C. Reagents are added in the following order: solid PPh3 (1.30 equiv per alcohol), dropwise addition of DEAD (1.33 equiv per alcohol), and then dropwise addition of diphenylphosphoryl azide (1.40 equiv per alcohol). The reaction mixture is stirred at 0 °C for 2.3 hours, then concentrated under reduced pressure. Biotage purification furnishes General Compound S.

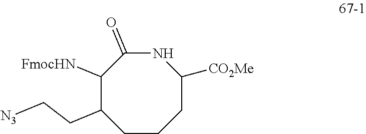

Compound 67-1 is prepared from Compound 66-1 using General Procedure S.

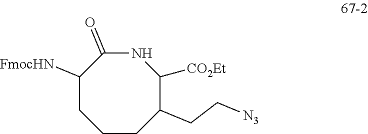

Compound 67-2 is prepared from Compound 66-2 using General Procedure S.

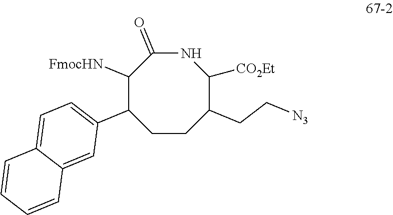

Compound 67-3 is prepared from Compound 66-3 using General Procedure S.

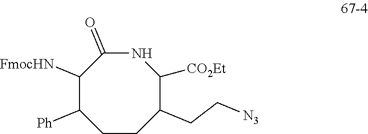

Compound 67-4 is prepared from Compound 66-4 using General Procedure S.

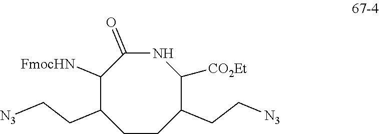

Compound 67-5 is prepared from Compound 66-5 using General Procedure S.

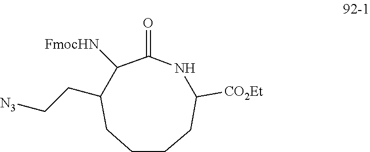

Compound 92-1 is prepared from Compound 90-1 using General Procedure S.

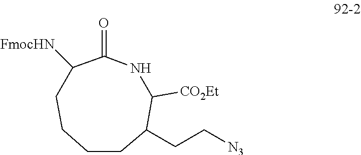

Compound 92-2 is prepared from Compound 90-2 using General Procedure S.

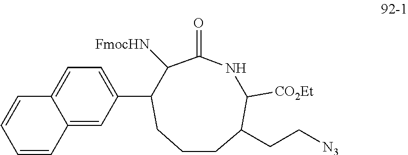

Compound 92-3 is prepared from Compound 90-3 using General Procedure S.

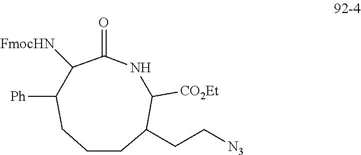

Compound 92-4 is prepared from Compound 90-4 using General Procedure S.

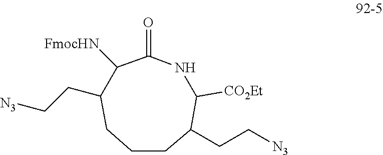

Compound 92-5 is prepared from Compound 90-5 using General Procedure S.

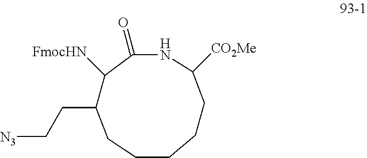

Compound 93-1 is prepared from Compound 91-1 using General Procedure S.

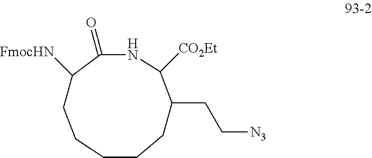

Compound 93-2 is prepared from Compound 91-2 using General Procedure S.

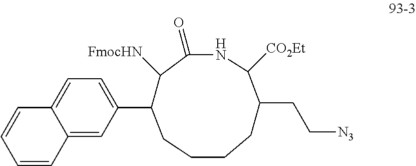

Compound 93-3 is prepared from Compound 91-3 using General Procedure S.

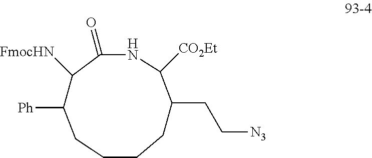

Compound 93-4 is prepared from Compound 91-4 using General Procedure S.

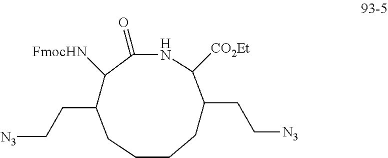

Compound 93-5 is prepared from Compound 91-5 using General Procedure S.

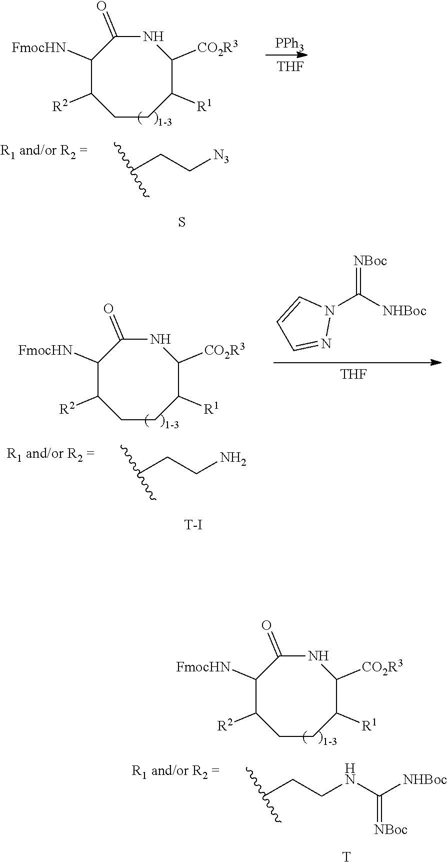

### Example 34: Compounds Made with General Procedure T

General Procedure T: Solid triphenylphosphine (3.5 equiv per azide) is added to a solution of General Compound U (1.0 equiv) in THF (20 vol) at room temperature. After stirring for 1.5 h, H₂O (0.5 mL) is added, and the reaction vessel was heated at 50 °C for 3 days. The organic solvent is removed under reduced pressure and the residue is purified by Biotage purification, providing the corresponding free amine.

To a solution of General Compound T-I (1.0 equiv) in THF (20 vol) at room temperature is added N,N′-di-tert-butoxycarbonyl-1H-pyrazole-1-carboxamidine (1.5 equiv per amine). After stirring for 12 hours at the same temperature, the mixture is poured into water and the resulting solution is extracted with Et₂O. The combined organic extracts are dried over MgSO₄, filtered, and concentrated in vacuo. The residue is purified by Biotage Purification to afford General Compound T.

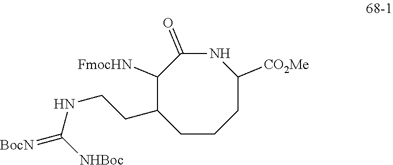

Compound 68-1 is prepared from Compound 67-1 using General Procedure T.

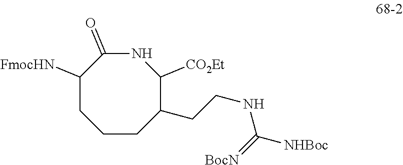

Compound 68-2 is prepared from Compound 67-2 using General Procedure T.

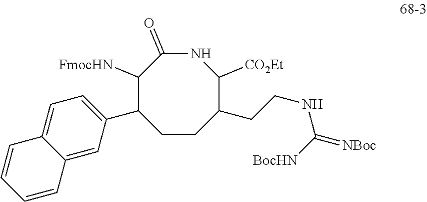

Compound 68-3 is prepared from Compound 67-3 using General Procedure T.

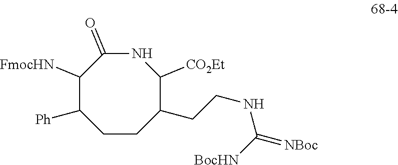

Compound 68-4 is prepared from Compound 67-4 using General Procedure T.

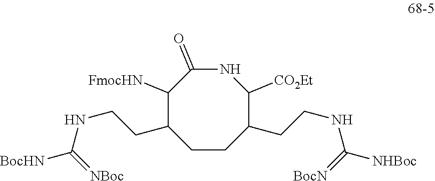

Compound 68-5 is prepared from Compound 67-5 using General Procedure T.

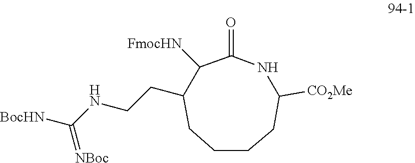

Compound 94-1 is prepared from Compound 92-1 using General Procedure T.

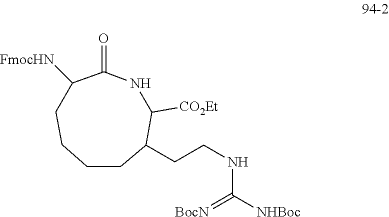

Compound 94-2 is prepared from Compound 92-2 using General Procedure T.

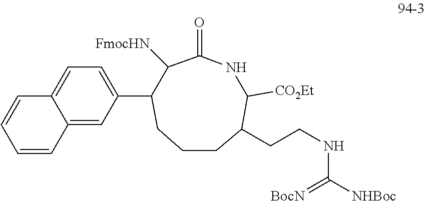

Compound 94-3 is prepared from Compound 92-3 using General Procedure T.

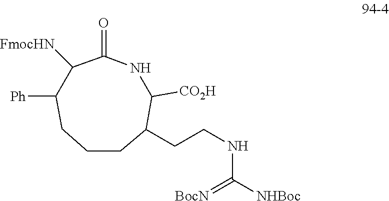

Compound 94-4 is prepared from Compound 92-4 using General Procedure T.

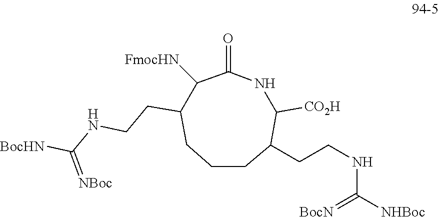

Compound 94-5 is prepared from Compound 92-5 using General Procedure T.

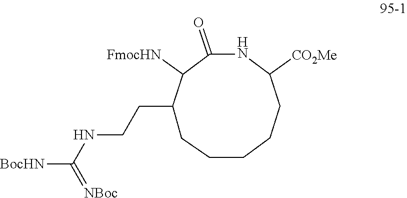

Compound 95-1 is prepared from Compound 93-1 using General Procedure T.

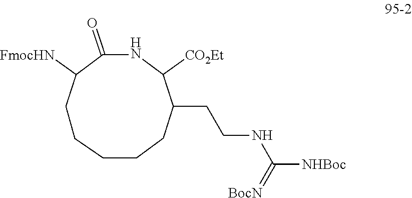

Compound 95-2 is prepared from Compound 93-2 using General Procedure T.

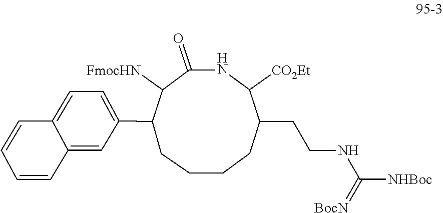

Compound 95-3 is prepared from Compound 93-3 using General Procedure T.

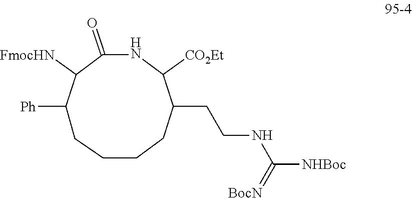

Compound 95-4 is prepared from Compound 93-4 using General Procedure T.

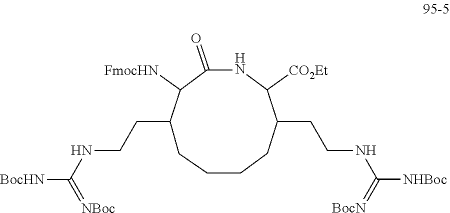

Compound 95-5 is prepared from Compound 93-5 using General Procedure T.

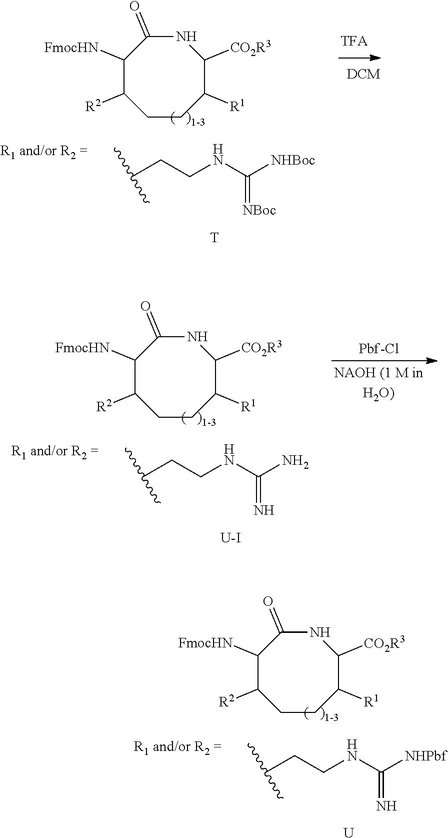

### Example 35: Compounds Made with General Procedure U

General Procedure U: TFA (10 vols) is added to a solution of General Compound T (1.0 equiv) in CH₂Cl₂ (10 vols) at 0 °C and the mixture is stirred for 10 hours at the same temperature. The end of reaction was checked by UPLC of the reaction mixture. After removal of the solvent and excess TFA under reduced pressure, General Compound U-I is used to the next step without any purification.

3N NaOH aqueous solution (5 vols) and N-(2,2,4,6,7-pentamethyldihydrobenzofuran-5-sulfomyl chloride (1.5 equiv per guanidine) are sequentially added to General Compound U-I (1.0 equiv) in THF (20 vols) at 0 °C. The solution is allowed to warm to roam temperature and is stirred for 12 hours. To the solution is added water and the mixture is acidified by 1N KHSO₄ aqueous solution to pH 2. The mixture is extracted with EtOAc. The combined organic extracts are dried over MgSO₄, filtered, and concentrated in vacuo. The residue is purified by column chromatography to afford General Compound U.

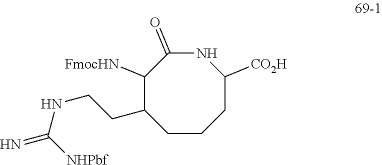

Compound 69-1 is prepared from Compound 68-1 using General Procedure U.

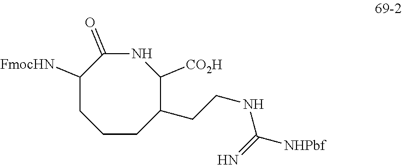

Compound 69-2 is prepared from Compound 68-2 using General Procedure U.

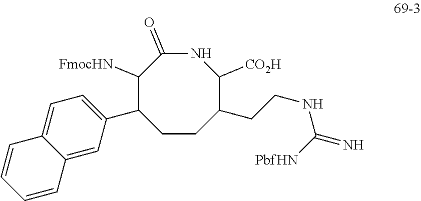

Compound 69-3 is prepared from Compound 68-3 using General Procedure U.

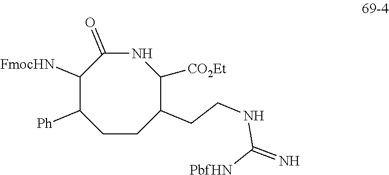

Compound 69-4 is prepared from Compound 68-4 using General Procedure U.

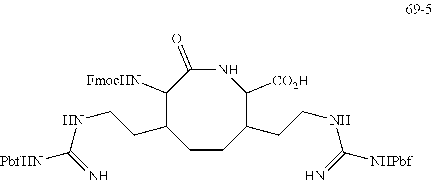

Compound 69-5 is prepared from Compound 68-5 using General Procedure U.

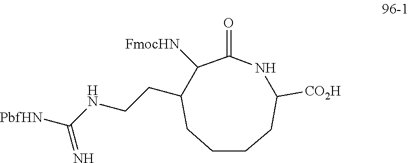

Compound 96-1 is prepared from Compound 94-1 using General Procedure U.

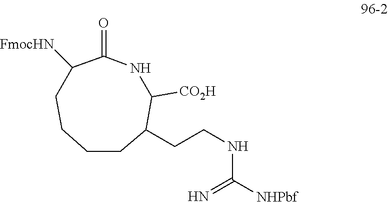

Compound 96-2 is prepared from Compound 94-2 using General Procedure U.

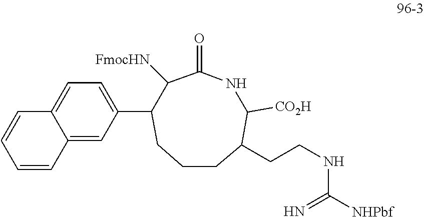

Compound 96-3 is prepared from Compound 94-3 using General Procedure U.

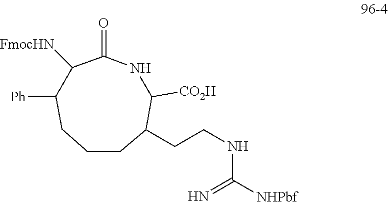

Compound 96-4 is prepared from Compound 94-4 using General Procedure U.

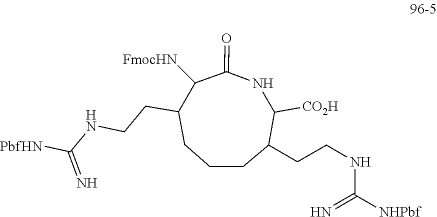

Compound 96-5 is prepared from Compound 94-5 using General Procedure U.

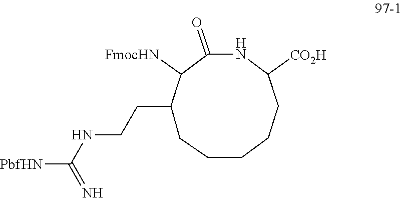

Compound 97-1 is prepared from Compound 95-1 using General Procedure U.

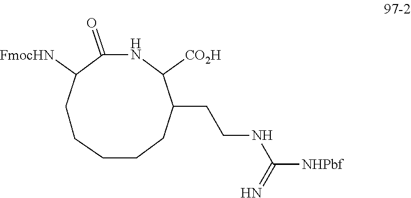

Compound 97-2 is prepared from Compound 95-2 using General Procedure U.

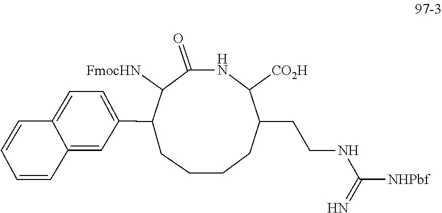

Compound 97-3 is prepared from Compound 95-3 using General Procedure U.

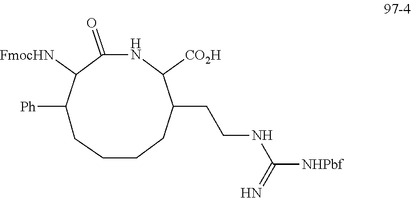

Compound 97-4 is prepared from Compound 95-4 using General Procedure U.

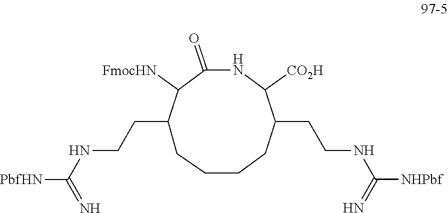

Compound 97-5 is prepared from Compound 95-5 using General Procedure U.

### Example 36: Making Compound 133

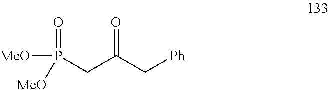 [SMILES: COP(=O)(CC(=O)Cc1ccccc1)OC]

Compound 133: The β-ketophosphonates are synthesized using the procedure from Ivan Kolarovic, A., Tetrahedron 2015, 71, 8876.

### Example 37: Making Compound 134

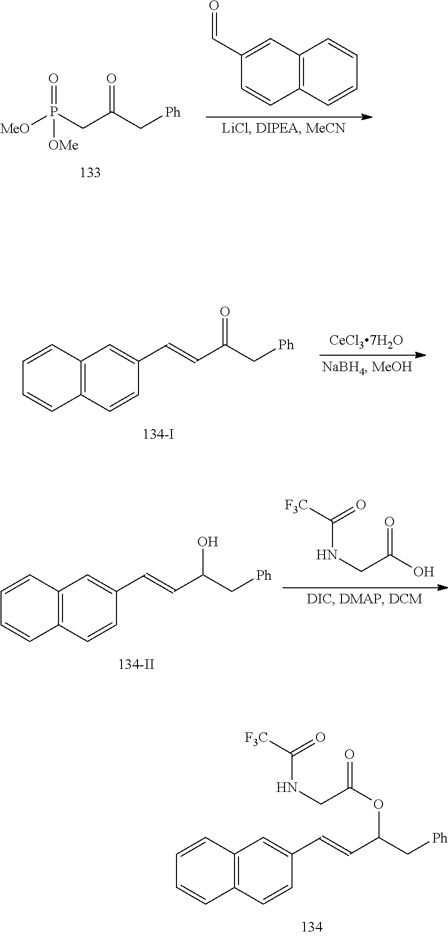 [SMILES: COP(=O)(CC(=O)Cc1ccccc1)OC ; O=Cc1ccc2ccccc2c1 ; O=C(/C=C/c1ccc2ccccc2c1)Cc1ccccc1 ; OC(/C=C/c1ccc2ccccc2c1)Cc1ccccc1 ; O=C(O)CNC(=O)C(F)(F)F ; O=C(CNC(=O)C(F)(F)F)OC(/C=C/c1ccc2ccccc2c1)Cc1ccccc1]

Compound 134: The aldehyde (1.0 equiv) in acetonitrile (10 vols) is added to PGP-44C3 the 133 (1.4 eq) and lithium chloride (2.0 equiv), it is then cooled in an ice bath, and DIPEA (4.0 equiv) is added, and kept stirring at 0 °C. The reaction is stirred until it is deemed completed by LCMS or TLC and is then partitioned between EA/sat. NaHCO₃, then back extracted once. The organic layer is washed with half saturated brine, dried over Na₂SO₄, and concentrated. Biotage purification affords Compound 134-I.

To a solution of Compound 134-I (1.0 equiv) and CeCl₃·7H2O (1.2 equiv) in MeOH (20 vols) is added sodium borohydride (1.2 equiv) portion wise at 0 °C, and the mixture is stirred for 30 minutes. The mixture is quenched with saturated aqueous NH₄Cl solution, and the solvent is removed by evaporation. The mixture is extracted with Et₂O, and the organic layer is combined, dried over Na₂SO₄, and concentrated under reduced pressure. The residue is purified by Biotage purification to giving Compound 134-II.

A mixture of Compound 134-II (1.0 equiv), (2,2,2-trifluoroacetyl)glycine (1.1 equiv) and DMAP (0.10 equiv) is taken into DCM (12 vols) to give a slightly yellow suspension. It is cooled in an ice-bath and DIC (1.1 equiv) is added in portions. The mixture was slowly allowed to warm to room temperature and stirred overnight. The mixture is taken into EtOAc and subsequently washed with NaHCO₃ (sat)×2, brine (150 mL), dried over sodium sulfate, and concentrated. The residue is purified via Biotage Purification to afford General Compound 134.

### Example 38: Making Compound 135

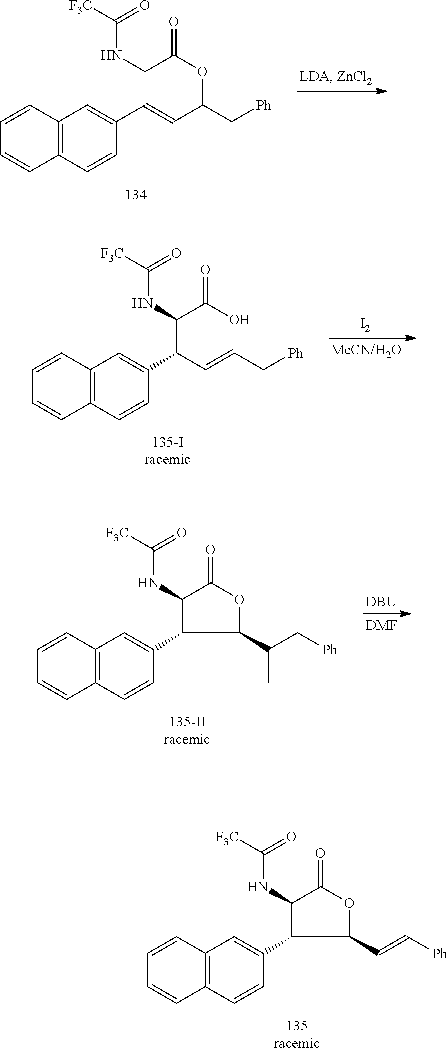

Compound 135: Claisen rearrangement: Zinc Chloride (1.5 equiv) is melted into clear liquid using a propane burner under house vacuum. It was cooled to room temperature and added to THF (5 vols). A solution of LDA in THF (1M, 4 equiv) is added to the solution of ZnCl₂-THF, followed by dropwise addition of a solution of Compound 134 (1.0 equiv) in THF (5 vols) at −78 °C, during which the mixture turns dark red, and then to lighter red when the addition is mostly complete. The solution is slowly allowed to warm to room temperature and stirred overnight. The reaction is quenched with 1 M HCl. The mixture is extracted with MTBE twice, dried, and concentrated. Compound 135-I compound is taken on to the next step as is.

Iodolactonization: Compound 135-I (1.0 equiv) is then added to a solution of acetonitrile (10 vols) and water (1 vol), diiodine (3.0 equiv) is added. The dark red solution is stirred at room temperature overnight. The reaction is quenched with aqueous sodium thiosulfate, extracted with ethyl acetate, dried, concentrated, and Biotage Purification to give Compound 135-II.

Elimination: To a sealed tube containing diazabicyclo[5.4.0]undec-7-ene (1.2 equiv) is added a solution of Compound 135-II (1.0 equiv) in anhydrous DMF (10 vols) under a nitrogen atmosphere. The reaction mixture is heated at 60 °C for 2 hours. The mixture is quenched with water and the organic materials are extracted with diethyl ether three times. The combined organic layers are washed with brine, dried over MgSO₄, and filtered. The volatile materials are removed, and the resulting residue is purified by Biotage Purification to give Compound 135.

### Example 39: Making Compound 136

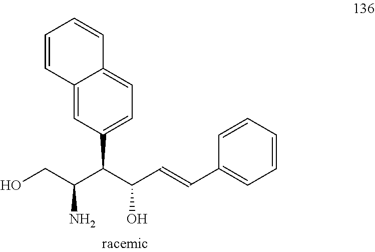 [SMILES: N[C@@H](CO)[C@@H](c1ccc2ccccc2c1)[C@@H](O)/C=C/c1ccccc1]

Compound 136: A solution of Compound 135 (1.0 equiv) in ethanol (10 vols) is cooled in an ice bath and treated with sodium borohydride (1.1 equiv) in portions. The mixture is slowly allowed to warm to room temperature and quenched with NaHCO₃ (sat). The mixture is concentrated to half of the original volume and the rest is extracted with ethyl acetate and washed with brine. The combined aqueous phases are back extracted with ethyl acetate and washed with brine. The combined organic phases are concentrated to give the desired amino alcohol, which was used without purification. The above crude is taken into methanol (10 vols), water (0.5 vols), and treated with potassium carbonate (5 equiv). It is stirred at room temperature overnight. The mixture is concentrated, combined with Celite 521 suspended in DCM, filtered, and concentrated. The residue is passed through a plug of silica gel rinsing with 20% methanol in DCM (conditioned with 1v/v % concentrated ammonia) The filtrate is concentrated and purified by Biotage Purification to give the desired amino alcohol.

### Example 40: Making Compound 137

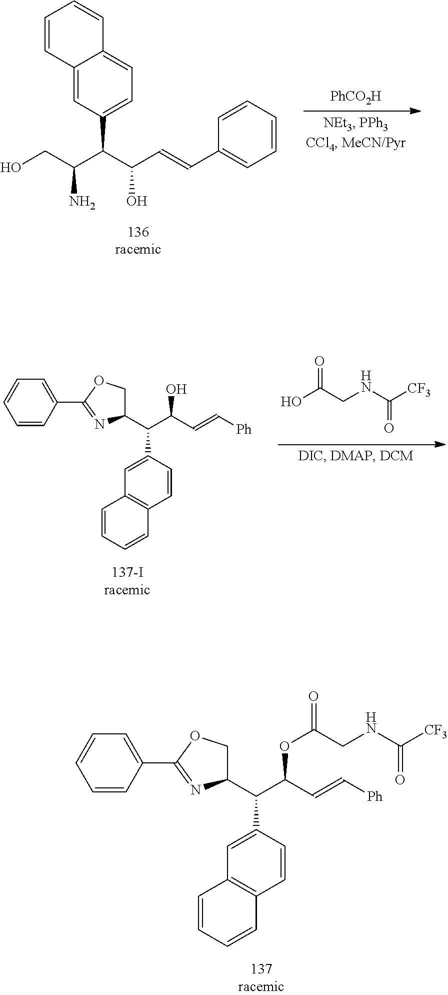 [SMILES: "PhCO2H" ; N[C@@H](CO)[C@@H](c1ccc2ccccc2c1)[C@@H](O)/C=C/c1ccccc1 ; O[C@@H](/C=C/c1ccccc1)[C@H](c1ccc2ccccc2c1)[C@@H]1COC(c2ccccc2)=N1 ; O=C(O)CNC(=O)C(F)(F)F ; O=C(CNC(=O)C(F)(F)F)O[C@@H](/C=C/c1ccccc1)[C@H](c1ccc2ccccc2c1)[C@@H]1COC(c2ccccc2)=N1]

Compound 137: To a solution of benzoic acid (1.0 equiv) and Compound 136 (1.0 equiv) is added triethylamine (1.0 equiv) and 3 ml (3 vols) CCl₄ in acetonitrile-pyridine (5 vols). A solution of triphenylphosphine (1.0 equiv) in acetonitrile-pyridine (5 vols) is added within 3 hours. Stirring continued for 1 hour to complete the formation of the intermediate amide. After addition of triethylamine (2.0 equiv) a solution of triphenylphosphine (2.0 equiv) in acetonitrile-pyridine (5 vols) is added within 3 hours, whereupon the reaction turned dark. After 18 hours overnight, the reaction mixture is evaporated, and the dark sticky residue stirred with toluene and ice-cold 2N NaOH. The aqueous phase is extracted with toluene three times, dried with Na₂SO₄, and evaporated. Biotage Purification gives the Compound 137-I.

A mixture of Compound 137-I (1.0 equiv), (2,2,2)-trifluoroacetyl)glycine (1.0 equiv) and DMAP (0.1 equiv) is taken into DCM (12 vols) and cooled in an ice-bath, followed by the addition of DIC (1.1 equiv) added in portions. The mixture is slowly allowed to warm to room temperature and stirred overnight. The mixture is taken into ethyl acetate/MTBE (1:1), washed twice with NaHCO₃ (sat), brine, dried over sodium sulfate, and concentrated to give the crude residue. It is taken into isopropanol and heated to 80 CC to give a homogeneous solution, which is then slowly allowed to warm to room temperature. The solid is collected by filtration and washed with isopropanol to give Compound 137.

### Example 41: Making Compound 138

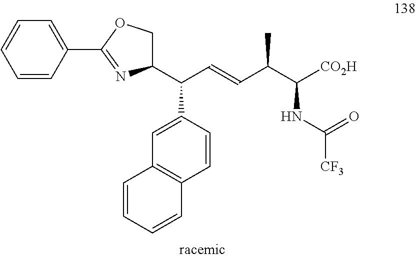 [SMILES: C[C@H](/C=C/[C@H](c1ccc2ccccc2c1)[C@@H]1COC(c2ccccc2)=N1)[C@H](NC(=O)C(F)(F)F)C(=O)O]

Compound 138: Zinc Chloride (1.5 equiv) is melted into clear liquid using a propane burner under house vacuum. It was cooled to room temperature and taken into THF (5 vols). A solution of LDA in THF (1M, 4.0 equiv) is added the solution of ZnCl₂-THF, followed by dropwise addition of a solution of Compound 137 (1.0 equiv) in THF (5 vols) at −78 °C, during which the mixture turns dark red, and then to lighter red when the addition is mostly complete. The solution is slowly allowed to warm to room temperature and stirred overnight. The reaction is quenched with 1 M HCl. The mixture is extracted with MTBE twice, dried, and concentrated to afford Compound 138. The compound is of sufficient purity to take to the next step.

### Example 42: Making Compound 139

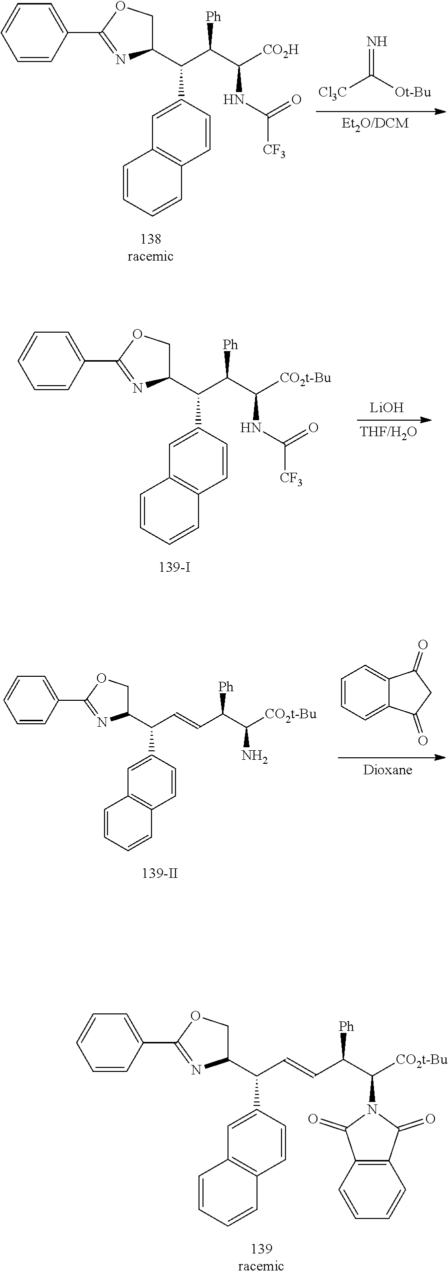

Compound 139: Tert-butyl-2,2,2-trichloroacetimidate (5.0 equiv) is added to a stirred solution Compound 138 (1.0 equiv) in dry ether (5 vols) and dry DCM (5 vols) under nitrogen and the resulting reaction mixture is stirred at room temperature over 2 days. The reaction is poured into NaHCO₃ (sat) and water (1:1) and extracted with DCM (three times). The combined organic extract is dried over sodium sulfate, concentrated under reduced pressure, and the resulting residue is purified by Biotage purification to give Compound 139-I.

Lithium hydroxide monohydrate (3.0 equiv) and water (2 vols) are added to a solution of Compound 139-I (1.0 equiv) in THF (10 vols). The reaction mixture temperature is warmed to 20-30 °C, and the reaction mixture is stirred for 10 hours. Upon reaction completion, water is added to the mixture, the compound is extracted with methyl tert-butyl ether four times, and the aqueous layer is removed. The organic layers are combined and washed with water. The organic layer is evaporated under reduced pressure and purified via Biotage purification to yield Compound 139-II.

Compound 139-II (1.0 equiv) in dioxane (10 vols) is added to a solution of phthalic anhydride (1.0 equiv) in dioxane (10 vols). The reaction is stirred until deemed complete by UPLC. The solvent is evaporated under reduced pressure. The resulting residue is purified by Biotage purification to afford Compound 139.

### Example 43: Making Compound 140

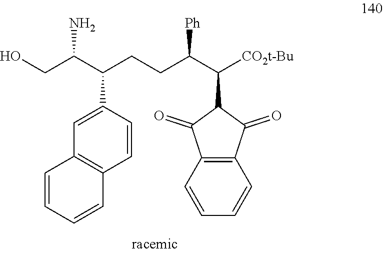 [SMILES: CC(C)(C)OC(=O)[C@@H](C1C(=O)c2ccccc2C1=O)[C@@H](CC[C@H](c1ccc2ccccc2c1)[C@@H](N)CO)c1ccccc1]

Compound 140: Compound 139 (1.0 equiv) is dissolved in a mixture of methanol (75 vols) and water (25 vols). To this solution is added approximately of 10% Pd/C (25% weight/weight) and the reaction mixture is treated with 1 atmosphere of hydrogen and allowed to react for 4 hours at room temperature. After 4 hours, the reaction mixture is filtered over a pad of Celite and washed three times with methanol. The organics are combined, and the solvent is removed under reduced pressure to yield Compound 140.

### Example 44: Making Compound 141

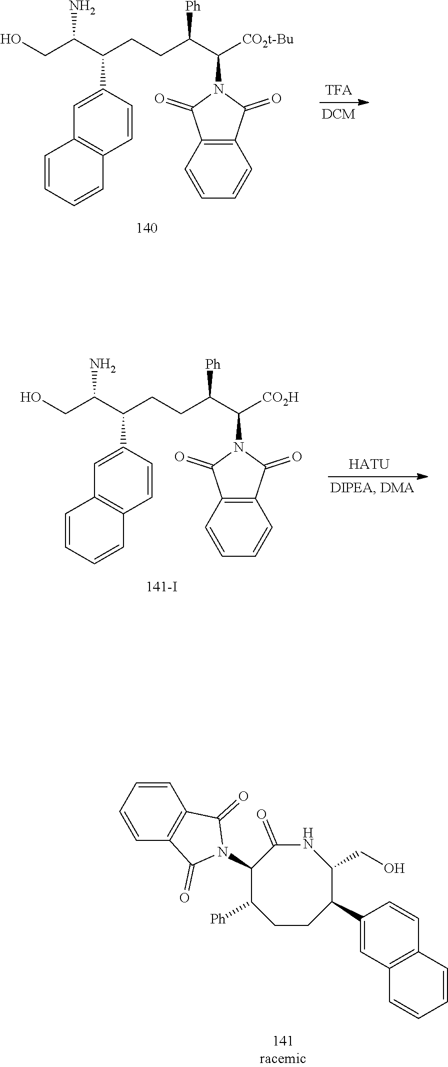

Compound 141: TFA (10 vols) is added to a solution of Compound 140 (1.0 equiv) in CH₂Cl₂ (10 vols) at 0 °C and the mixture is stirred for 10 hours at room temperature. After removal of the solvent and excess TFA under reduced pressure, Compound 141-I is used in the next step without any further purification.

DIPEA (5 equiv) and HATU (2.0 equiv) are added to a solution of Compound 141-I (1.0 equiv) in DMA (10 vols). The mixture is stirred overnight at room temperature. Water and NaHCO₃ (sat). are added to the reaction mixtures. The aqueous layer is extracted with ethyl acetate three times. The combined organic layers are washed with NaHCO₃ (sat), water, and brine. The organic layer is dried over sodium sulfate, filtered, and evaporated to give a residue. The residue is purified via Biotage Purification to afford Compound 141.

### Example 45: Making Compound 142

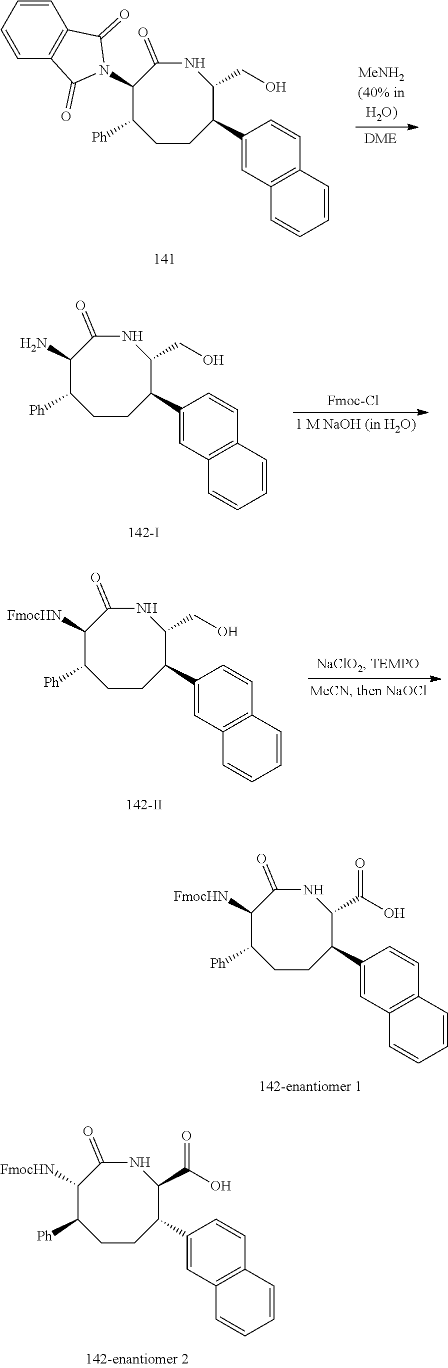

Compound 142: Compound 142 (1.0 equiv) and 1,2-dimethoxyethane (20 vols) are charged is a flask under nitrogen. A 40% aqueous methylamine solution (5 vols) is added and is stirred for 5 hours at roam temperature. Upon reaction completion, a 10% aqueous sodium hydroxide solution is added, and the mixture is stirred for 16 hours. The resulting solid is collected by filtration. The filter cake is washed with water and then dried to yield Compound 142-I.

Fmoc-Cl (1.2 equivalents) is added to a solution of Compound 142-I (1.0 equiv) in 1 N NaOH (20 vols) at 0 °C. After 24 hours at room temperature, the reaction mixture is diluted with H₂O and the pH is adjusted to 2-3 with 2 N HCl. The aqueous layer is extracted with EtOAc three times. The combined organic phases are washed with brine (1×10 mL). After drying over Na₂SO₄ and filtration, the solvent is removed by rotary evaporation to afford Compound 142-II.

A solution of NaClO₂ (80% w/w, 2.8 equiv) in phosphate buffer (0.67 N, pH=7, 10 vols) is added dropwise to a stirred mixture of Compound 142-II (1.0 equiv) and TEMPO (1.50 equiv) in MeCN (15 vols) at 0 °C. NaOCl (6% bleach, 0.7 equiv) is added dropwise to the mixture over 30 minutes. The reaction mixture is allowed to stir at 0 °C for 3 hours. The reaction is quenched with saturated Na₂SO₃ and allowed to warm to room temperature. The biphasic solution is acidified with 12 M HCl (pH approximately 2), and the aqueous layer is extracted with DCM three times. The combined organic layer is dried with MgSO₄, filtered, and concentrated. The material is purified via Biotage Purification to afford 142-enantiomer 1 and 142-enantiomer 2.

### Example 46: Making Compound 143

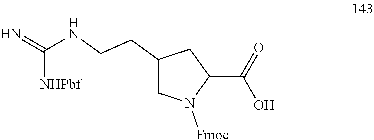

Compound 143: The guanidinylated Proline substrates are synthesized using the procedure from Ishiguro, M., J. Med. Chem. 2004, 47, 489.

### Example 47: Compounds Made with General Procedure V

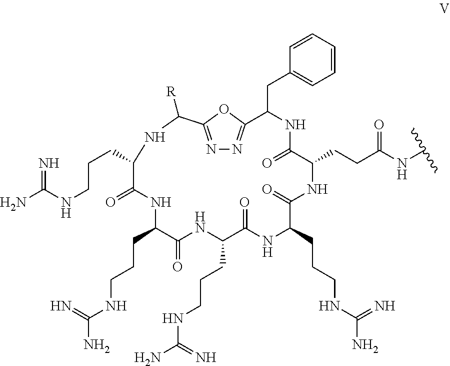

General Procedure V: The peptide (1.0 eq) is dissolved in a mixture of 1:1 Dichloroethane:Acetonitrile (40 vols). The aldehyde (1.5 eq) and (N-isocyanimino)triphenylphosphorane (1 eq) are added to the reaction mixture. After cyclization and reverse phase purification the pure fractions are pooled and lyophilized to afford the two diastereomers.

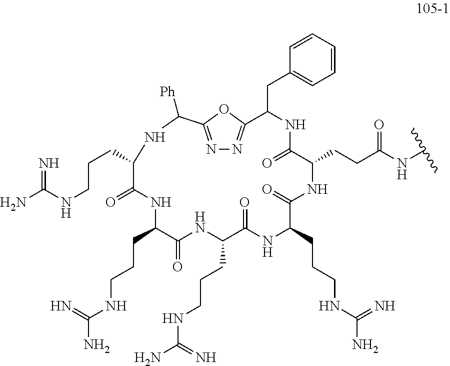 [SMILES: *NC(=O)CC[C@@H]1NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(c2ccccc2)c2nnc(o2)C(Cc2ccccc2)NC1=O]

Compound 105-1 is prepared from the corresponding peptide substrate using General Procedure V.

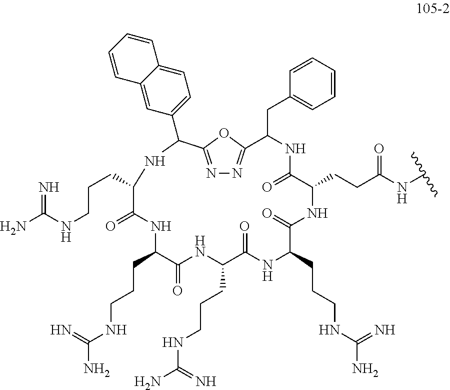 [SMILES: *NC(=O)CC[C@@H]1NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(c2ccc3ccccc3c2)c2nnc(o2)C(Cc2ccccc2)NC1=O]

Compound 105-2 is prepared from the corresponding peptide substrate using General Procedure V.

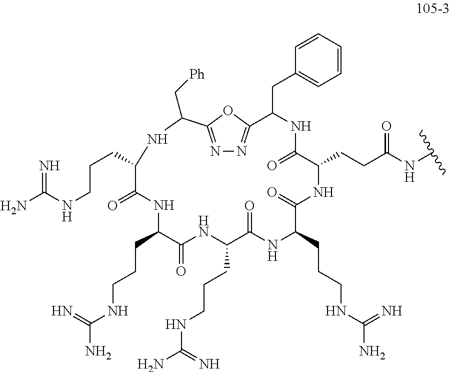 [SMILES: *NC(=O)CC[C@@H]1NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(Cc2ccccc2)c2nnc(o2)C(Cc2ccccc2)NC1=O]

Compound 105-3 is prepared from the corresponding peptide substrate using General Procedure V.

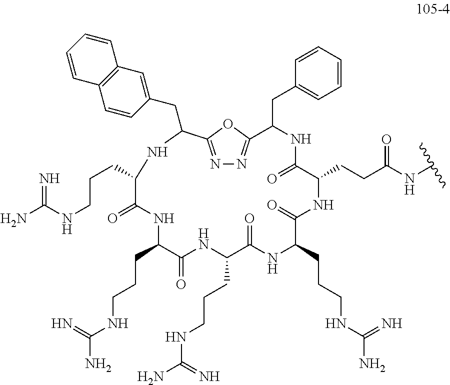 [SMILES: *NC(=O)CC[C@@H]1NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(Cc2ccc3ccccc3c2)c2nnc(o2)C(Cc2ccccc2)NC1=O]

Compound 105-4 is prepared from the corresponding peptide substrate using General Procedure V.

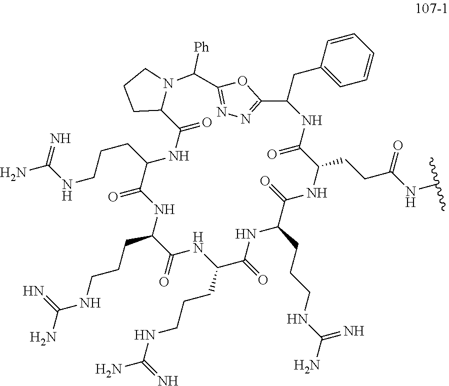 [SMILES: *NC(=O)CC[C@@H]1NC(=O)[C@@H](CCCNC(=N)N)NC(=O)[C@H](CCCNC(=N)N)NC(=O)[C@@H](CCCNC(=N)N)NC(=O)C(CCCNC(=N)N)NC(=O)C2CCCN2C(c2ccccc2)C2=NN=C(O2)C(Cc2ccccc2)NC1=O]

Compound 107-1 is prepared from the corresponding peptide substrate using General Procedure V.

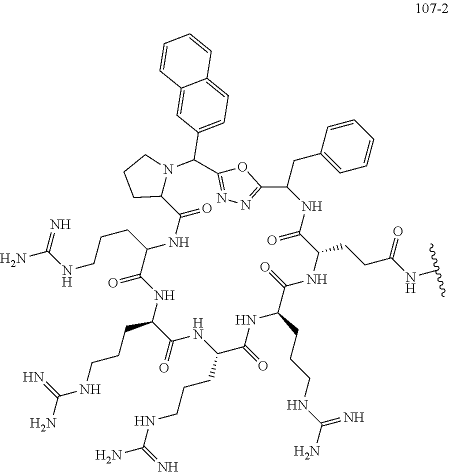 [SMILES: *NC(=O)CC[C@@H]1NC(=O)[C@@H](CCCNC(=N)N)NC(=O)[C@H](CCCNC(=N)N)NC(=O)[C@@H](CCCNC(=N)N)NC(=O)C(CCCNC(=N)N)NC(=O)C2CCCN2C(c2ccc3ccccc3c2)C2=NN=C(O2)C(Cc2ccccc2)NC1=O]

Compound 107-2 is prepared from the corresponding peptide substrate using General Procedure V.

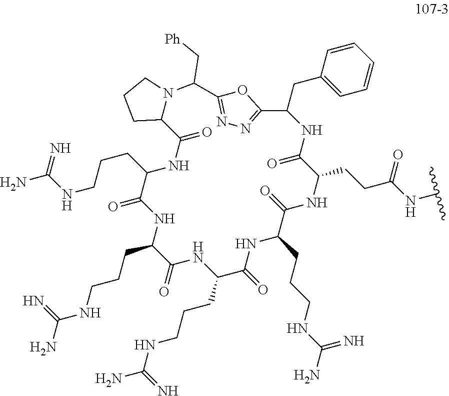 [SMILES: *NC(=O)CC[C@@H]1NC(=O)[C@@H](CCCNC(=N)N)NC(=O)[C@H](CCCNC(=N)N)NC(=O)[C@@H](CCCNC(=N)N)NC(=O)C(CCCNC(=N)N)NC(=O)C2CCCN2C(Cc2ccccc2)C2=NN=C(O2)C(Cc2ccccc2)NC1=O]

Compound 107-3 is prepared from the corresponding peptide substrate using General Procedure V.

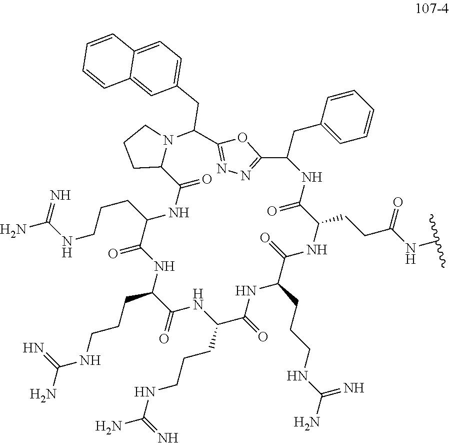 [SMILES: *NC(=O)CC[C@@H]1NC(=O)[C@@H](CCCNC(=N)N)NC(=O)[C@H](CCCNC(=N)N)NC(=O)[C@@H](CCCNC(=N)N)NC(=O)C(CCCNC(=N)N)NC(=O)C2CCCN2C(Cc2ccc3ccccc3c2)C2=NN=C(O2)C(Cc2ccccc2)NC1=O]

Compound 107-4 is prepared from the corresponding peptide substrate using General Procedure V.

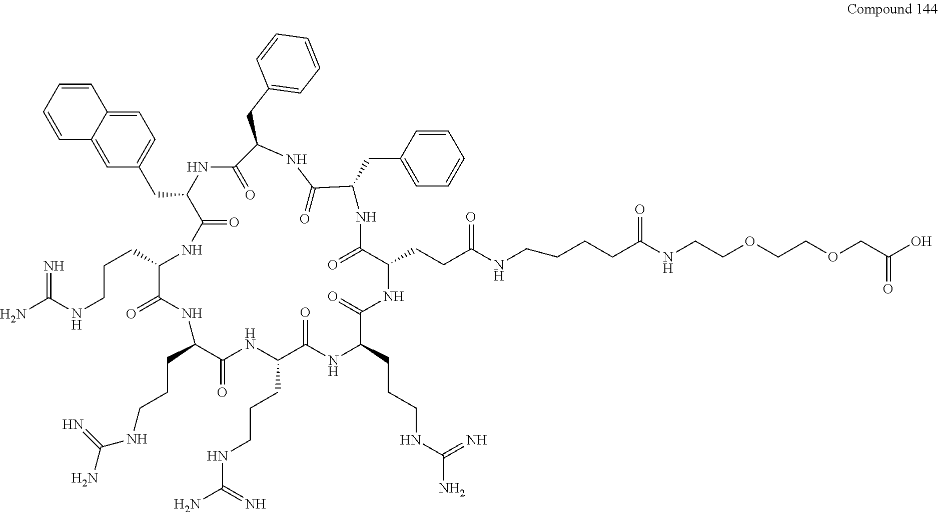 [SMILES: N=C(N)NCCC[C@@H]1NC(=O)[C@H](Cc2ccc3ccccc3c2)NC(=O)[C@@H](Cc2ccccc2)NC(=O)[C@H](Cc2ccccc2)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

Compound 144 is prepared by standard Fmoc solid-phase synthesis protocols described in Example 13.

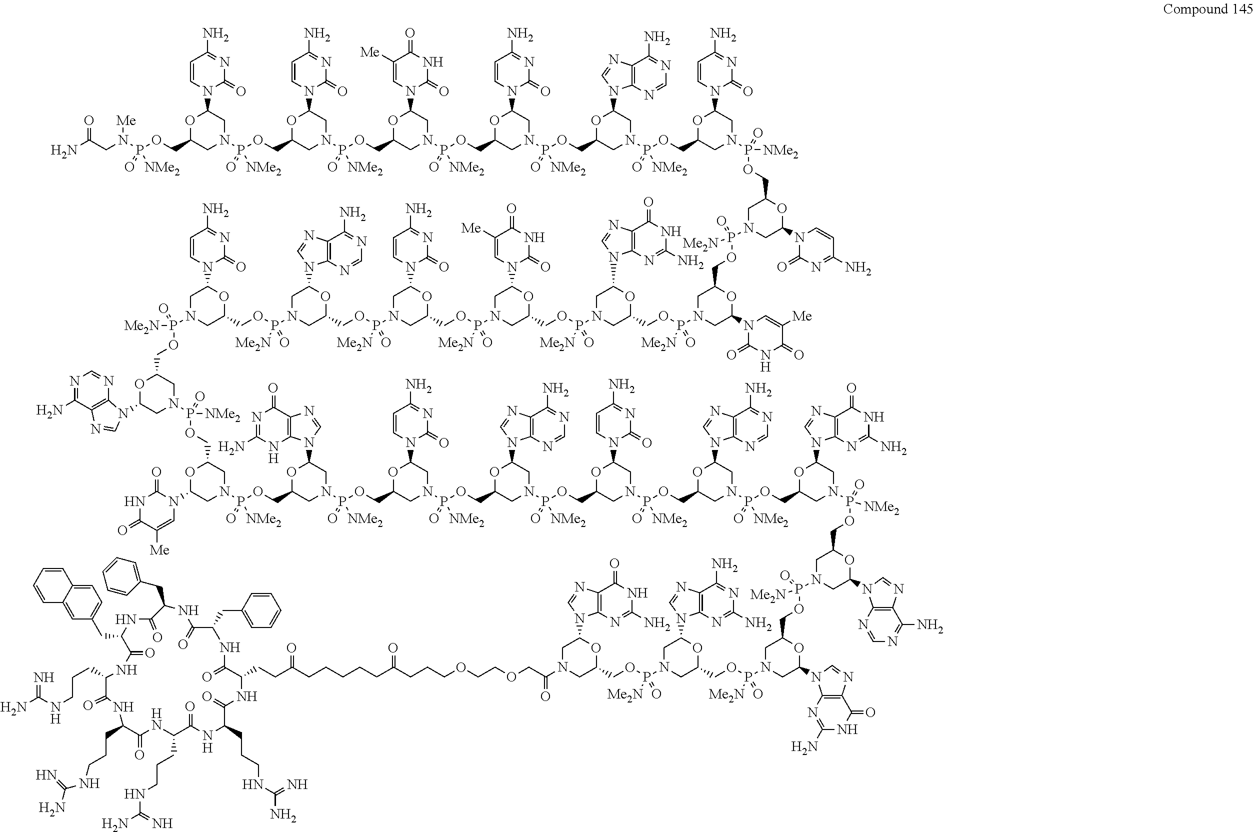

Compound 145 is prepared from PMO-002 and Compound 144 by using General Procedure D.

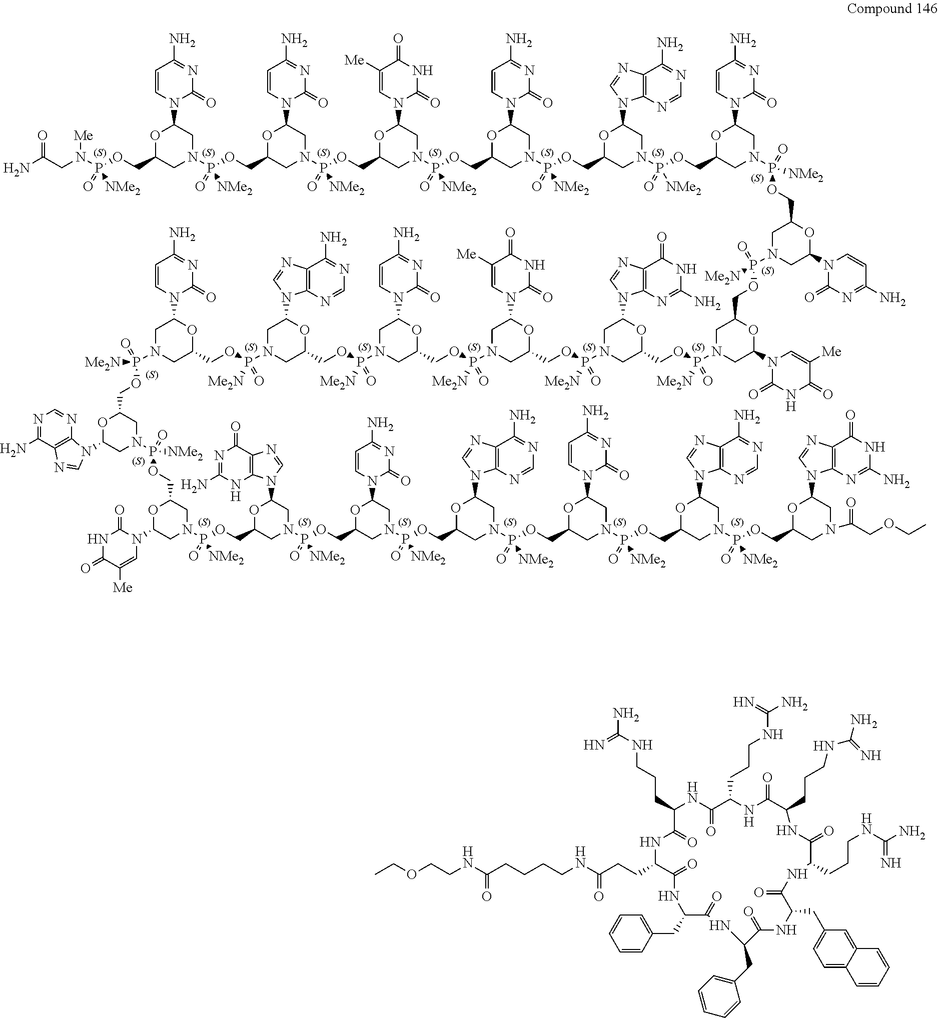

Compound 146 is prepared from PMO-424 and Compound 144 by using General Procedure D.

### Example 48: Making Compounds 166/167 and 168/169

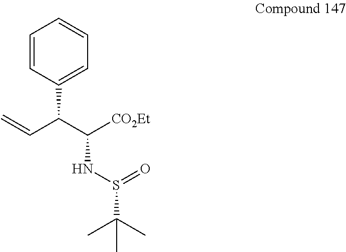 [SMILES: C=C[C@H](c1ccccc1)[C@@H](N[S@](=O)C(C)(C)C)C(=O)OCC]

Compound 147: 15.8 g, (72% yield) of Compound 147 was prepared from (E)-(3-bromoprop-1-en-1-yl)benzene (14.4 ml, 97.4 mmol) and ethyl (S,E)-2-((tert-butylsulfinyl)imino)acetate (10.0 g, 48.7 mmol) using General Procedure H. ¹H NMR (400 MHz, CDCl₃) δ ppm 7.29-7.35 (2H, m), 7.25 (1H, m, J=6.90 Hz), 7.19 (2H, d, J=7.75 Hz), 6.09 (1H, ddd, J=17.60, 9.00 Hz), 5.13-5.22 (2H, m), 4.13-4.26 (3H, m), 3.92 (1H, br d, J=9.38 Hz), 3.71 (1H, t, J=7.94 Hz), 1.28 (3H, t, J=7.07 Hz), 1.08 (9H, s); LRMS (ESI, m/z) Calculated for [C₁₇H₂₅NO₃S+Na]⁺ 346.1. Found 346.1.

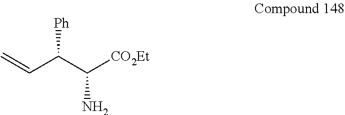 [SMILES: C=C[C@H](c1ccccc1)[C@@H](N)C(=O)OCC]

7.7 g (100% yield) of Compound 148 was prepared from Compound 147 (11.4 g, 35.2 mmol) using General Procedure I. ¹H NMR (400 MHz, CDCl₃) δ ppm 7.28-7.38 (2H, m), 7.21-7.26 (3H, m), 6.12 (1H, ddd, J=16.88, 9.13, 8.50 Hz), 5.10-5.22 (2H, m), 4.16 (2H, q, J=7.13 Hz), 3.80 (1H, d, J=7.63 Hz), 3.64 (1H, t, J=7.94 Hz), 1.87 (2H, br s), 1.25 (3H, t, J=7.13 Hz); LRMS (ESI, m/z) Calculated for [C₁₃H₁₇NO₂+H]⁺ 220.14. Found 220.1.

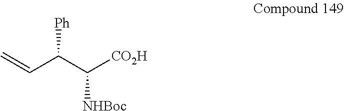 [SMILES: C=C[C@H](c1ccccc1)[C@@H](NC(=O)OC(C)(C)C)C(=O)O]

10.0 g (86% yield) of Compound 149 was prepared from Compound 148 (7.7 g, 34.3 mmol) using General Procedure J. ¹H NMR (400 MHz, CDCl₃) δ ppm 7.28-7.37 (3H, m), 7.21-7.26 (2H, m), 6.11 (1H, ddt, J=17.50, 8.80, 8.80, 8.80 Hz), 5.23 (2H, br d, J=13.51 Hz), 4.81 (1H, br d, J=8.38 Hz), 4.68 (1H, br t, J=6.88 Hz), 3.76-3.95 (1H, m), 1.40 (9H, s); LRMS (ESI, m/z) Calculated for [C₁₈H₂₁NO₄+Na]⁺ 314.14. Found 314.1.

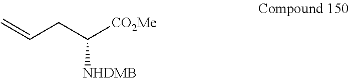

Compound 150 was synthesized using the procedure from Creighton, C. J., Bioorg. Med. Chem. 12, 2004, 4375. ¹H NMR (400 MHz, CHLOROFORM-d) δ ppm 7.12 (1H, d, J=7.88 Hz), 6.41-6.46 (2H, m), 5.67-5.79 (1H, m), 5.03-5.14 (2H, m), 3.80 (6H, s), 3.71-3.77 (1H, m), 3.62-3.71 (4H, m), 3.35 (1H, t, J=6.57 Hz), 2.42 (2H, br t, J=6.88 Hz), 1.20-1.35 (1H, m); LRMS (ESI, m/z) Calculated for [C₁₅H₂₁NO₄+Na]⁺ 302.14. Found 302.1.

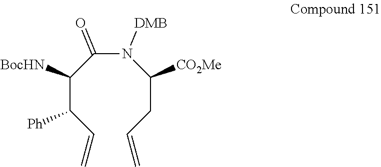

Compound 151: Compound 151 (940 mg, 83% yield) was prepared using General Procedure O from Compound 149 (4.90 g, 2.06 mmol) and Compound 150 (1.15 g, 4.12 mmol). ¹H NMR (400 MHz, CHLOROFORM-d) δ ppm 7.17-7.24 (5H, m), 7.04-7.17 (2H, m), 6.26-6.39 (2H, m), 5.96-6.10 (1H, m), 5.44-5.51 (1H, m), 4.92-5.17 (4H, m), 4.70-85 (2H, m), 4.33-4.42 (1H, m), 4.16 (1H, d, J=15.76 Hz), 3.82-4.03 (1H, m), 3.66-3.76 (6H, m), 3.53 (3H, s), 2.69 (1H, dt, J=14.07, 6.85 Hz), 2.25 (1H, dt, J=14.38, 7.44 Hz), 1.20-1.28 (9H, m); LRMS (ESI, m/z) Calculated for [C₃₁H₄₀N₂O₇+H]⁺ 553.3. Found 553.4.

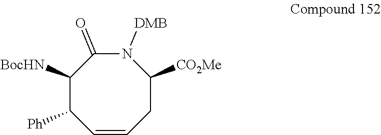

Compound 152: Compound 151 (61.0 mg, 0.11 mmol, 1 equiv) was dissolved in toluene (53 ml, 870 vol) and the solution was sparged for 30 min with N2. Ruthenium, [1,3-bis-(2,4,6-trimethylphenyl)-2-imidazolinylidene]dichloro[[2-(1-methylethoxy)phenyl]methylene](41.6 mg, 0.066 mmol, 0.5 equiv) was added in two portions over an hour. The resulting solution was refluxed for 6 h. The residue was concentrated and purified via Biotage purification to afford Compound 152 (37 mg, 0.071 mmol, 63.9% yield). ¹H NMR (400 MHz, CHLOROFORM-d) δ ppm 7.27 (6H, m, J=3.63 Hz), 6.39-6.49 (2H, m), 5.80-5.89 (1H, m), 5.24-5.58 (2H, m), 5.08 (1H, br t, J=10.44 Hz), 4.84 (1H, br d, J=14.38 Hz), 4.34-4.45 (2H, m), 3.76-3.85 (6H, m), 3.67 (3H, s), 3.38-3.52 (1H, m), 3.00-3.30 (1H, m), 2.79-2.94 (1H, m), 1.22-1.38 (9H, m); LRMS (ESI, m/z) Calculated for [C₂₉H₃₆N₂O₇+H]⁺ 525.3. Found 525.5.

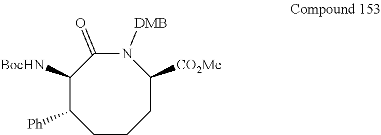

Compound 153: Compound 152 (1.17 g, 2.23 mmol, 1 equiv) was dissolved in MeOH (34.2 ml, 30 vols) followed by the addition of Pd-C (0.598 g, 50% wt, 10% Pd basis). H₂ was bubbled in the solution for 5 min and the reaction was left under an atm of H₂. The reaction was stirred overnight. The suspension was filtered through Celite and washed with water and MeOH. The residue was then concentrated and purified by Biotage purification to afford Compound 153 (900 mg, 1.71 mmol, 77% yield). ¹H NMR (400 MHz, CHLOROFORM-d) δ ppm 7.25-7.29 (5H, m), 7.14-7.21 (1H, m), 6.44-6.52 (2H, m), 5.25 (1H, d, J=9.63 Hz), 5.09 (1H, d, J=14.13 Hz), 4.79 (1H, t, J=11.00 Hz), 4.41 (1H, d, J=14.13 Hz), 4.35 (1H, t, J=4.94 Hz), 3.80-3.84 (9H, m), 3.11 (1H, br dd, J=11.44, 2.69 Hz), 2.22-2.40 (1H, m), 1.93-2.12 (1H, m), 1.76-1.87 (1H, m), 1.68-1.76 (1H, m), 1.57 (9H, s), 1.39-1.55 (1H, m); LRMS (ESI, m/z) Calculated for [C₂₉H₃₈N₂O₇+H]⁺ 527.3. Found 527.5.

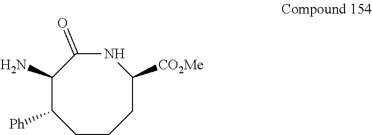 [SMILES: COC(=O)[C@H]1CCC[C@H](c2ccccc2)[C@@H](N)C(=O)N1]

Compound 154: Compound 153 (527 mg, 1.00 mmol, 1 equiv) was treated with a solution of triethylsilane (0.20 ml, 2 vols), water (2 ml, 4 vols) and TFA (0.10 ml, 2 vols). The reaction was permitted to proceed for 16 h after which the solvent was removed and the crude oil was treated with excess heptanes. The product was triturated in heptanes for 4 h and then collected. After 3 h, the solvent was removed by reduced pressure and the compound was purified by Biotage purification yielding Compound 154 (391 mg, 1.04 mmol, quant) as the TFA salt. LRMS (ESI, m/z) Calculated for [C₁₅H₂₀N₂O₃+H]⁺ 277.2. Found 277.8.

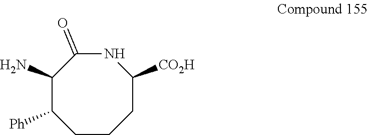 [SMILES: N[C@H]1C(=O)N[C@@H](C(=O)O)CCC[C@@H]1c1ccccc1]

Compound 155: Compound 154 (918 mg, 2.35 mmol, 1 equiv) was dissolved in THF (8 ml, 9 vol) and water (2 ml, 2 vol) at rt to which lithium hydroxide hydrate (493 mg, 11.8 mmol, 5 equiv) was added. The solution was stirred overnight and upon completion, the pH was adjusted with HCl (1M) until the pH was ~1-2. The solution was extracted with EtOAc×3, washed with brine, and the organics were dried with Na₂SO₄. The resulting solution was concentrated to dryness and analyzed without further purification to afford Compound 155 (472 mg, 77% yield). This material was of sufficient purity to proceed to the next step. LRMS (ESI, m/z) Calculated for [C₁₄H₁₈N₂O₃+H]⁺ 263.1. Found 263.8.

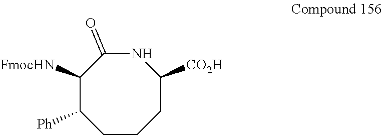

Compound 156: Compound 155 (472 mg, 1.80 mmol, 1 equiv) was dissolved in 1,4-dioxane (5 ml, 10 vol) and a solution of sodium bicarbonate (sat) (5 mL, 10 vol). (9H-fluoren-9-yl)methyl carbonochloridate (512 mg, 1.98 mmol, 1.1 equiv) was added and stirred overnight. The pH of the solution was adjusted to pH~1-2. The resulting aq phase was extracted with EA×3 and the organics were washed and concentrated. The resulting residue was purified via Reverse Phase Biotage Purification to afford Compound 156 (650 mg, 1.34 mmol, 75% yield). ¹H NMR (400 MHz, DMSO-d₆) δ ppm 12.59-13.21 (1H, br, s), 8.12-8.24 (1H, m), 7.85 (2H, brd, J=7.50 Hz), 7.49-7.61 (3H, m), 7.34-7.46 (4H, m), 7.19-7.34 (5H, m), 4.86 (1H, dd, J=11.88, 8.75 Hz), 4.64 (1H, d, J=4.25 Hz), 4.37-4.46 (1H, m), 4.02-4.12 (2H, m), 2.85 (1H, t, J=11.13 Hz), 2.00-2.19 (2H, m), 1.74-1.87 (2H, m), 1.51-1.68 (1H, m), 1.42 (1H, m); LRMS (ESI, m/z) Calculated for [C₂₉H₂₈N₂O₅+H]⁺ 485.2. Found 485.0.

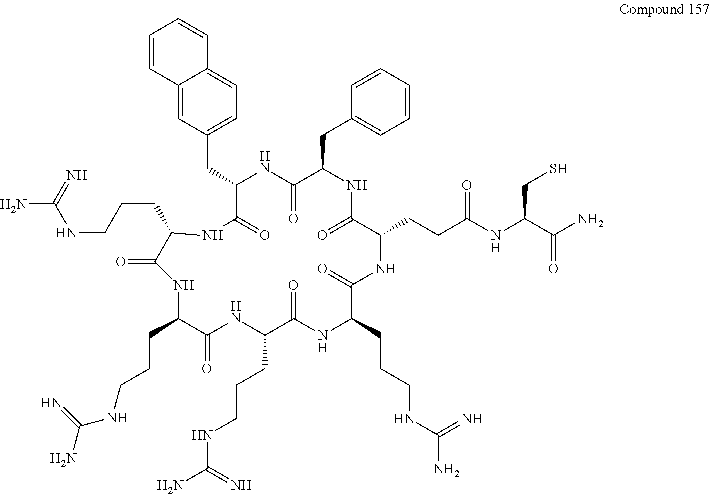 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)N[C@@H](CS)C(N)=O)NC(=O)[C@@H](CCCNC(=N)N)NC(=O)[C@H](CCCNC(=N)N)NC(=O)[C@@H](CCCNC(=N)N)NC1=O]

Compound 157 was prepared by standard Fmoc solid-phase synthesis protocols described in Example 13. MW=1200.44, LRMS (ESI, m/z): Calcd. m/z for [M+2H]²⁺ ion of C₅₄H₈₁N₂₁O₉S: 600.8. found: 600.5.

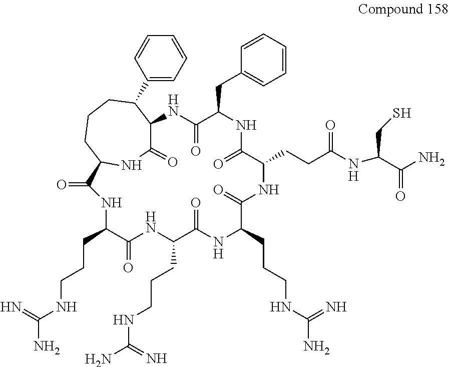 [SMILES: N=C(N)NCCC[C@@H]1NC(=O)[C@@H](CCCNC(=N)N)NC(=O)[C@H]2CCC[C@H](c3ccccc3)[C@@H](NC(=O)[C@@H](Cc3ccccc3)NC(=O)[C@H](CCC(=O)N[C@@H](CS)C(N)=O)NC(=O)[C@@H](CCCNC(=N)N)NC1=O)C(=O)N2]

Compound 158 was prepared by standard Fmoc solid-phase synthesis protocols described in Example 13. MW=1091.31, LRMS (ESI, m/z): Calcd. m/z for [M+2H]²⁺ ion of C₄₉H₇₄N₁₈O₉S: 546.29. found: 546.65.

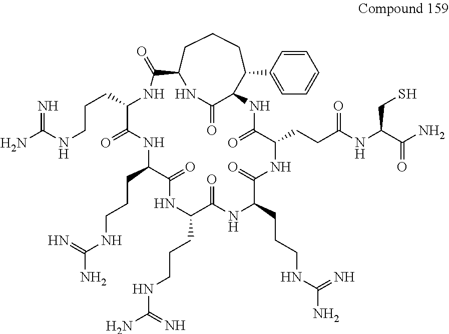 [SMILES: N=C(N)NCCC[C@@H]1NC(=O)[C@@H](CCCNC(=N)N)NC(=O)[C@H](CCCNC(=N)N)NC(=O)[C@H]2CCC[C@H](c3ccccc3)[C@@H](NC(=O)[C@H](CCC(=O)N[C@@H](CS)C(N)=O)NC(=O)[C@@H](CCCNC(=N)N)NC1=O)C(=O)N2]

Compound 159 was prepared by standard Fmoc solid-phase synthesis protocols described in Example 13. MW=1100.32, LRMS (ESI, m/z): Calcd. m/z for [M+2H]²⁺ ion of C₄₆H₇₇N₂₁O₉S: 550.8. found: 551.2.

General Procedure W: The select peptide (1 equiv) was dissolved in DMF (15 vols) followed by the addition of a solution of 4-methyl-2-oxo-2H-chromen-7-yl (4-(pyridin-2-yldisulfaneyl)butyl) carbonate or 2-((4-azidobutyl)disulfaneyl)pyridine (5 equiv) in THF (15 vols). The resulting mixture was stirred at room temperature overnight. The solution was submitted to prep-HPLC purification using the below purification conditions. The desired disulfide conjugate was returned.

### General Purification Conditions of Peptides by RP-HPLC:

| Preparative | | |
|---|---|---|
| Column | Waters, XBridge Prep C18 | |
| | OBD 5 μm, 10 × 100 mm | |
| Flow rate | 8 ml/min | |
| Column temperature | room temperature | |
| Solution A | 0.075% TFA in water | |
| Solution B | 0.075% TEA in AON | |
| | Time(min): | % B |
| Gradient | 0 | 10 |
| | 1 | 30 |
| | 7 | 30 |
| | 17 | 70 |
| | 20 | 70 |
| | 20.1 | 10 |
| | 25 | 10 |

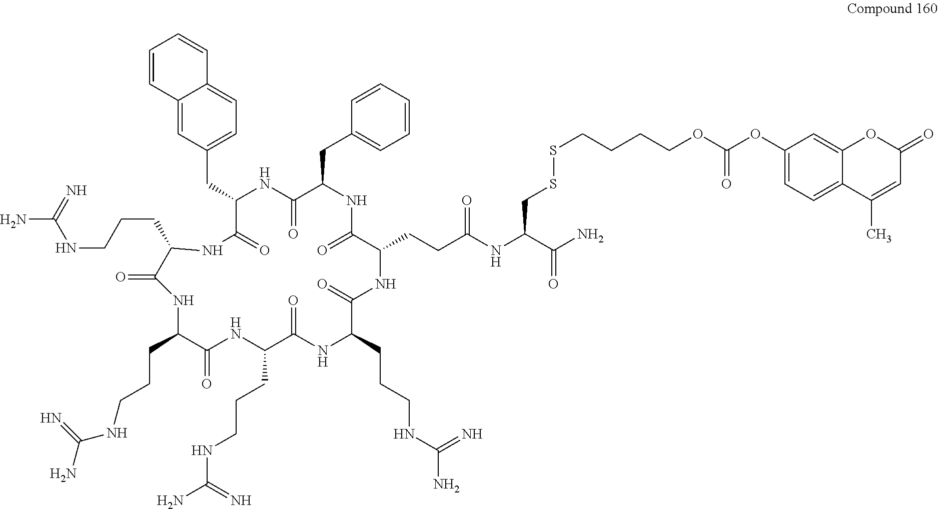 [SMILES: Cc1cc(=O)oc2cc(OC(=O)OCCCCSSC[C@H](NC(=O)CC[C@@H]3NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@H](Cc4ccc5ccccc5c4)NC(=O)[C@@H](Cc4ccccc4)NC3=O)C(N)=O)ccc12]

Compound 160: Compound 160 (3.95 mg, 48% yield) was prepared as a TFA salt by General Procedure W from a TFA salt of Compound 157 (5.0 mg; 4.2 μmol) and 4-methyl-2-oxo-2H-chromen-7-yl (4-(pyridin-2-yldisulfaneyl)butyl) carbonate. MW=1200.44, LRMS (ESI, m/z): Calcd. m/z for [M+2H]²⁺ ion of C₈₉H₉₅N₂1O₁₄S₂: 753.9. found: 753.9.

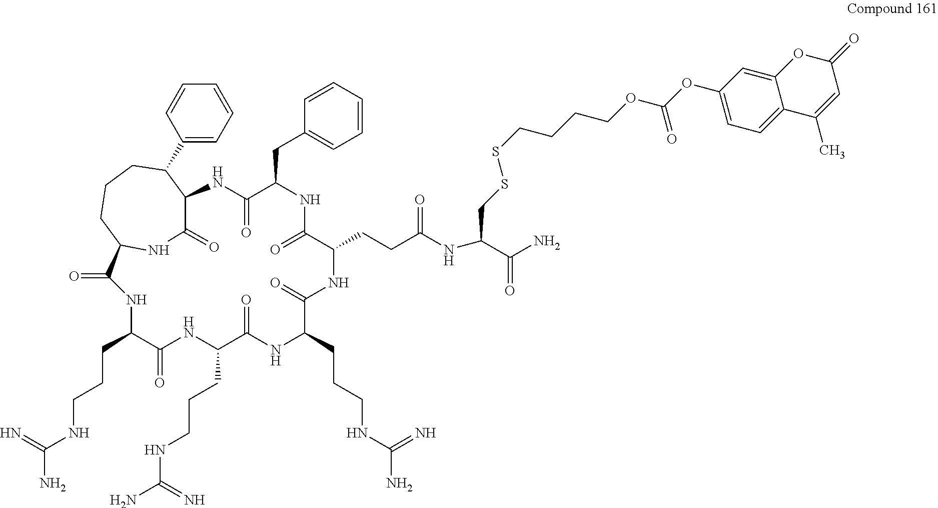 [SMILES: Cc1cc(=O)oc2cc(OC(=O)OCCCCSSC[C@H](NC(=O)CC[C@@H]3NC(=O)[C@@H](CCCNC(=N)N)NC(=O)[C@H](CCCNC(=N)N)NC(=O)[C@@H](CCCNC(=N)N)NC(=O)[C@H]4CCC[C@H](c5ccccc5)[C@@H](NC(=O)[C@@H](Cc5ccccc5)NC3=O)C(=O)N4)C(N)=O)ccc12]

Compound 161: Compound 161 (2.1 mg, 70% yield) was prepared as a TFA salt by General Procedure W from a TFA salt of Compound 158 (2.5 mg; 1.62 μmol) and 4-methyl-2-oxo-2H-chromen-7-yl (4-(pyridin-2-yldisulfaneyl)butyl) carbonate. MW=1397.64, LRMS (ESI, m/z):Calcd. m/z for [M+2H]²⁺ ion of C₆₄H₈₈N₁₈O₁₄S₂: 699.3. found: 699.4.

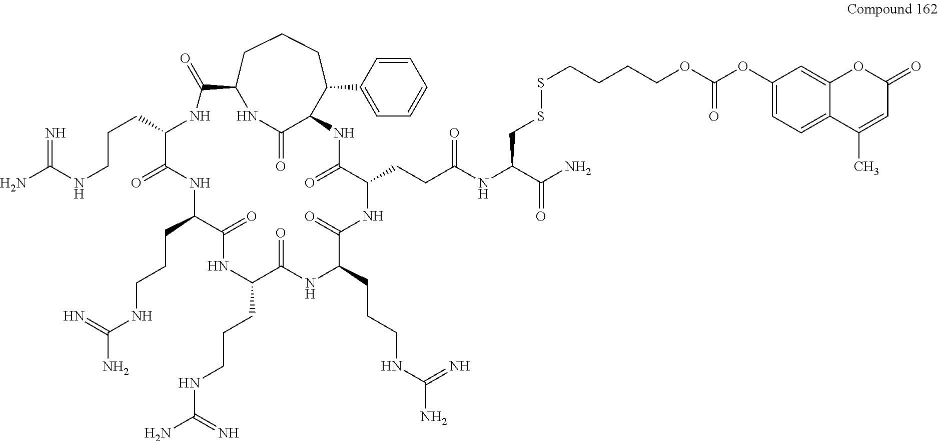 [SMILES: Cc1cc(=O)oc2cc(OC(=O)OCCCCSSC[C@H](NC(=O)CC[C@@H]3NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@H]4CCC[C@H](c5ccccc5)[C@@H](NC3=O)C(=O)N4)C(N)=O)ccc12]

Compound 162: Compound 162 (2.4 mg, 80% yield) was prepared as a TFA salt by General Procedure W from a TFA salt of Compound 159 (2.5 mg; 1.61 μmol) and 4-methyl-2-oxo-2H-chromen-7-yl (4-(pyridin-2-yldisulfaneyl)butyl) carbonate. MW=1406.65, LRMS (ESI, m/z): Calcd. m/z for [M+2H]²⁺ ion of C₆₁H₉₁N₂₁O₁₄S₂: 703.8. found: 703.9.

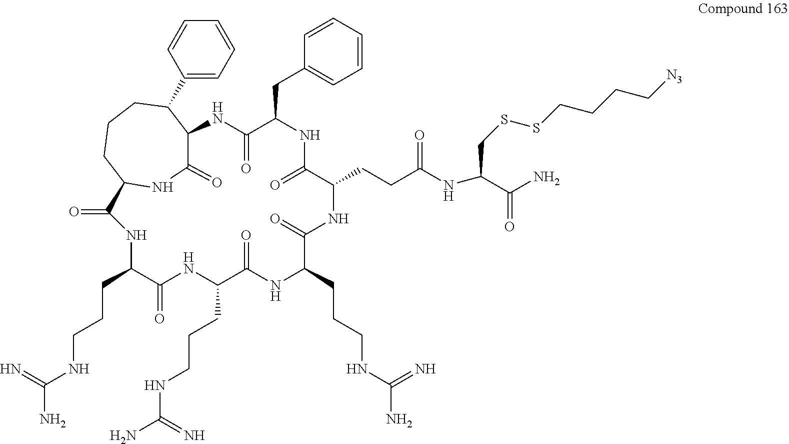 [SMILES: [N-]=[N+]=NCCCCSSC[C@H](NC(=O)CC[C@@H]1NC(=O)[C@@H](CCCNC(=N)N)NC(=O)[C@H](CCCNC(=N)N)NC(=O)[C@@H](CCCNC(=N)N)NC(=O)[C@H]2CCC[C@H](c3ccccc3)[C@@H](NC(=O)[C@@H](Cc3ccccc3)NC1=O)C(=O)N2)C(N)=O]

Compound 163: Compound 163 (1.06 mg, 36% yield) was prepared as a TFA salt by General Procedure W from a TFA salt of Compound 158 (2.7 mg; 1.75 μmol) and 2-((4-azidobutyl)disulfaneyl)pyridine. MW=1220.49, LRMS (ESI, m/z): Calcd. m/z for [M+H]⁺ ion of C₅₃H₈₁N₂₁O₉S₂: 1220.6. found: 1220.4.

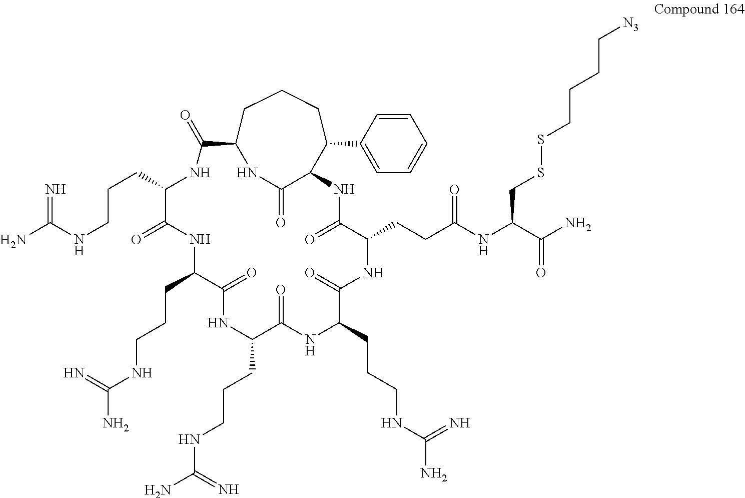 [SMILES: [N-]=[N+]=NCCCCSSC[C@H](NC(=O)CC[C@@H]1NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@H]2CCC[C@H](c3ccccc3)[C@@H](NC1=O)C(=O)N2)C(N)=O]

Compound 164: Compound 164 (1.24 mg, 44% yield) was prepared as a TFA salt by General Procedure W from a TFA salt of Compound 159 (2.6 mg; 1.67 μmol) and 2-((4-azidobutyl)disulfaneyl)pyridine. MW=1228.63, LRMS (ESI, m/z): Calcd. m/z for [M+H]⁺ ion of C₅₀H₈₄N₂₄O₉S₂: 1229.6. found: 1229.3.

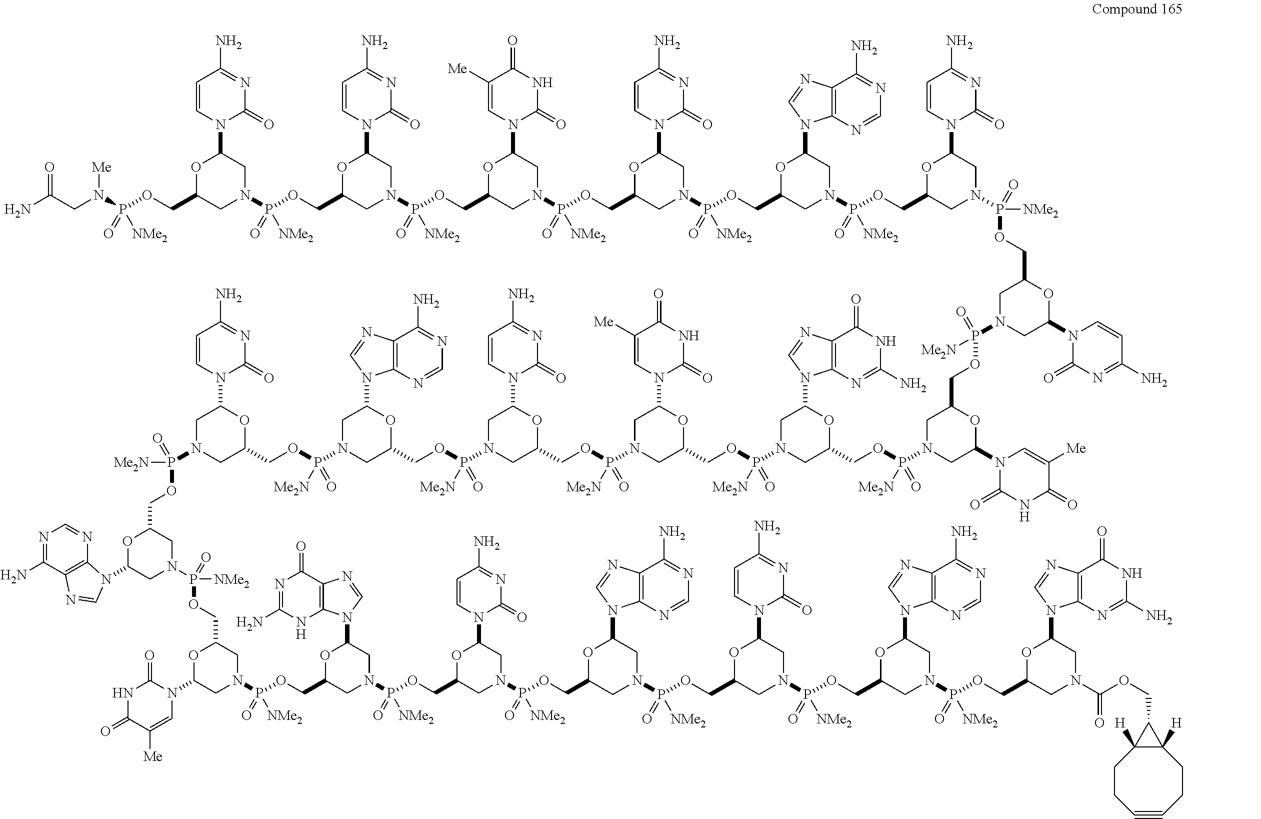

Compound 165: Compound 165 (25.6 mg, 99%) was prepared from PMO-424 (25.6 mg, 3.57 μmol) using carbamate conjugation protocols described in Example 12. MW=7185.239, LRMS (ESI, m/z): Calcd. m/z for [M+5H]⁵⁺ ion of C₂₅₇H₄₀₂N₁₁₉O₈₆P₂₁: 1437.9. found: 1437.6.

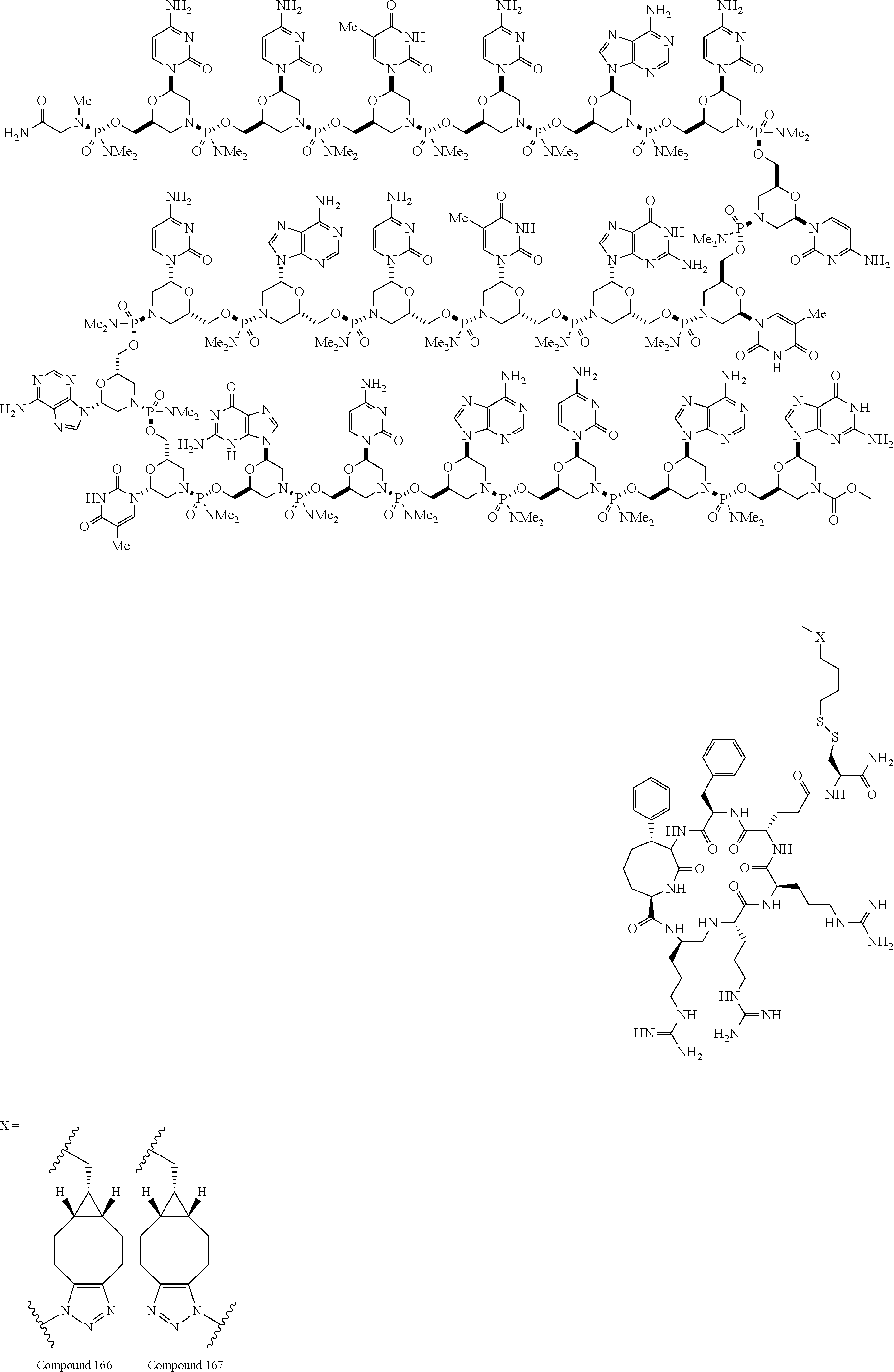

Compound 166 and Compound 167 (mixture of triazole Isomers): A mixture of Compound 166 and Compound 167 (3.25 mg, 93%) was prepared by General Procedure D from Compound 165 (3.0 mg, 0.418 μmol) and Compound 163 (1.05 mg, 0.626 μmol). MW=8405.728, LRMS: Calcd. m/z for [M+5H]⁵⁺ ion of C₃₁₀H₄₈₃N₁₄₀O₉₅P₂₁S₂: 1681.4. found: 1681.7.

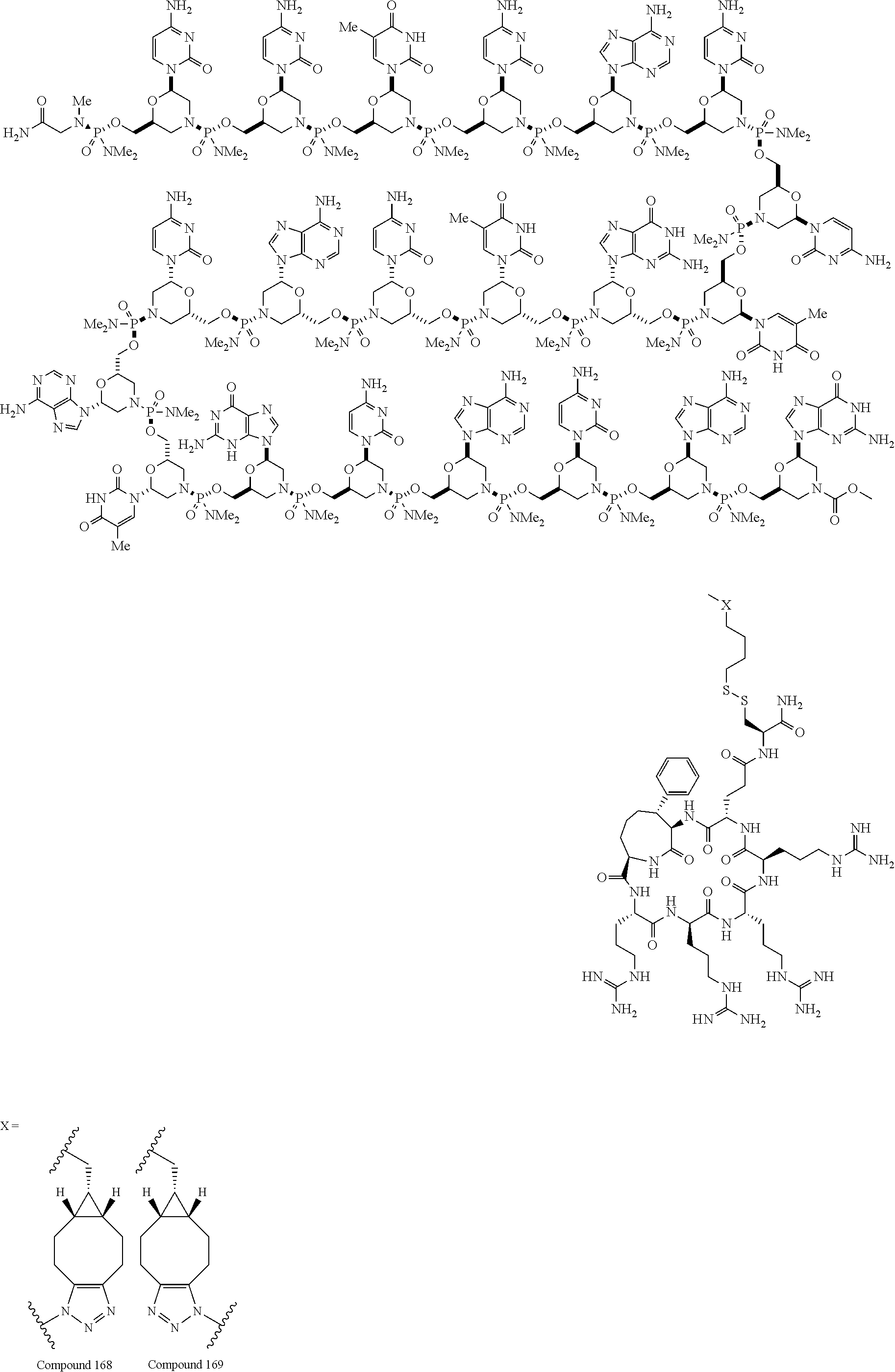

Compound 168 and Compound 169 (mixture of triazole Isomers): A mixture of Compound 168 and Compound 169 (3.67 mg, 90%) Was prepared by General Procedure D from Compound 165 (3.5 mg, 0.487 μmol) and Compound 164 (1.23 mg, 0.731 μmol). MW=8414.740, LRMS: Calcd. m/z for [M+5H]⁵⁺ ion of C₃₀₇H₄₈₆N₁₄₃O₉₅P₂₁S₂: 1683.6. found: 1683.5.

### Example 49: Making Compound 134

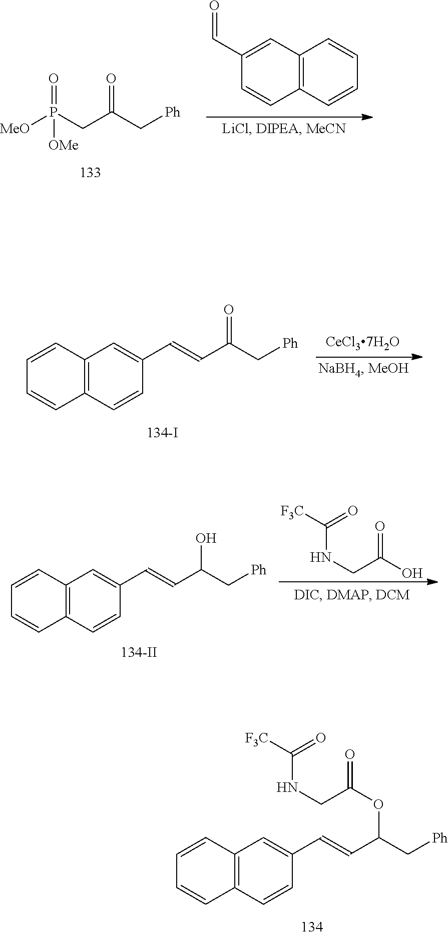 [SMILES: COP(=O)(CC(=O)Cc1ccccc1)OC ; O=Cc1ccc2ccccc2c1 ; O=C(/C=C/c1ccc2ccccc2c1)Cc1ccccc1 ; OC(/C=C/c1ccc2ccccc2c1)Cc1ccccc1 ; O=C(O)CNC(=O)C(F)(F)F ; O=C(CNC(=O)C(F)(F)F)OC(/C=C/c1ccc2ccccc2c1)Cc1ccccc1]

Compound 134: 2-napthaldehyde (3.00 g, 19.2, 1.0 equiv) in acetonitrile (30 mL, 10 vols) was added to a solution of 133 (7.27 g, 26.9 mmol, 1.4 eq) and lithium chloride (1.63 g, 38.4 mmol, 2.0 equiv) in acetonitrile (30 mL, 10 vols), and was then cooled in an ice bath. DIPEA (13.4 mL, 76.8 mmol, 4.0 equiv) was added, and kept stirring at 0 °C. The reaction was stirred until it was deemed completed by LCMS or TLC and was then partitioned between EA/NaHCO₃ (sat), and back extracted once. The organic layer was washed with half saturated brine, dried over Na₂SO₄, and concentrated. The resulting residue was purified via Biotage purification affording Compound 134-I (5.23 g, 38%)

To a solution of Compound 134-I (2.0 g, 9.00 mmol, 1.0 equiv) and CeCl₃·7H₂O (4.02 g, 10.7 mmol, 1.2 equiv) in MeOH (40 mL, 20 vols) was added sodium borohydride (408 mg, 10.7 mmol, 1.2 equiv) portion wise at 0 °C. The mixture was stirred for 30 minutes. The mixture was quenched with NH₄Cl (sat) solution, and the solvent was removed by evaporation. The mixture was extracted with MTBE, and the organic layer was combined, dried over Na₂SO₄, and concentrated under reduced pressure. The residue was purified by Biotage purification giving Compound 134-II (2.02 g, 94%).

A mixture of Compound 134-II (1.9 g, 6.93 mmol, 1.0 equiv), (2,2,2-trifluoroacetyl)glycine (1.30 g, 7.62 mmol, 1.1 equiv) and DMAP (85 mg, 0.69 mmol, 0.10 equiv) was taken into DCM (50 mL, 25 vols) to give a slightly yellow suspension. It was cooled in an ice-bath and DIC (1.2 mL, 7.62 mmol, 1.1 equiv) was added in portions. The mixture was slowly allowed to warm to room temperature and stirred overnight. The mixture was taken into EtOAc and subsequently washed with NaHCO₃ (sat)×2, brine (sat), dried over sodium sulfate, and concentrated. The residue was purified via Biotage Purification to afford General Compound 134 (2.4 g, 81%).

¹H NMR (400 MHz, CHLOROFORM-d) δ ppm 7.77-7.83 (3H, m), 7.74 (1H, s), 7.56 (1H, m, J=8.50 Hz), 7.44-7.51 (2H, m), 7.29-7.36 (2H, m), 7.23-7.26 (3H, m), 6.69-6.86 (2H, m), 6.29 (1H, dd, J=15.95, 7.44 Hz), 5.77 (1H, q, J=7.25 Hz), 4.00-4.18 (2H, m), 3.11 (2H, d, J=5.00 Hz); LRMS (ESI, m/z) Calculated for [C₂₄H₂₀F₃NO₃—C₄H₃F₃NO₃]⁺ 257.1. Found 257.0.

### Example 50: Making Compound 135

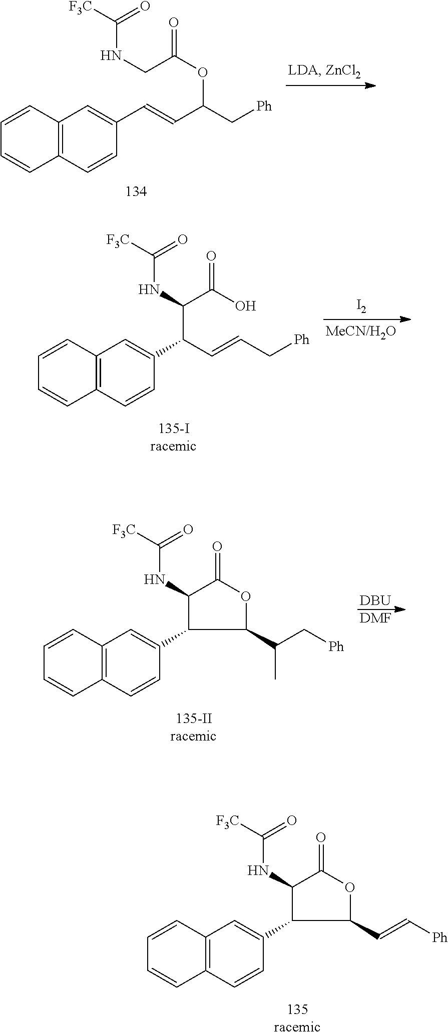

Compound 135: Claisen rearrangement: Zinc Chloride (3.84 mL, 7.3 mmol, 1.9 M in 2-Me-THF, 1.5 equiv) was added to an oven dried flask under nitrogen. A solution of LDA in THF (14.6 mL, 14.6 mmol, 1M, 4 equiv) was added to the solution of ZnCl₂, followed by dropwise addition of a solution of Compound 134 (2.08 g, 4.87 mmol, 1.0 equiv) in THF (20 vols) at −78 °C, during which the mixture turned dark red, and then to lighter red when the addition was mostly complete. The solution was slowly allowed to warm to room temperature and stirred overnight. The reaction was quenched with 1 M HCl. The mixture was extracted with MTBE twice, dried with Na₂SO₄, filtered and concentrated. Compound 135-I (2.1 g, 100%) was taken on to the next step crude.

Iodolactonization: Compound 135-I (1 g, 2.34 mmol, 1 equiv) and a mixture of MeCN (19 ml, 19 vols) and water (1 ml, 1 vol) were charged into a flask. The mixture was stirred for 5-10 min, and the resulting white suspension was charged with diiodine (1.19 g, 4.68 mmol. 2.0 equiv) at the same temperature. The reaction mixture was stirred at 20-30 °C overnight. The content was monitored by UPLC. Upon reaction completion, the reaction was quenched with a solution of sodium thiosulfate in water; the reaction turned from a dark brown to pale yellow color solution. The compound was extracted with EA, and the organic layers were combined and washed with water and brine. The organic layer was concentrated under reduced pressure to yield an inseparable mixture of Compound 135-II and the 6-endo product.

Elimination: To a Nitrogen charged flask containing diazabicyclo[5.4.0]undec-7-ene (0.32 mL, 2.13 mmol, 1.2 equiv) was added a solution of Compound 135-II and the 6-endo product (980 mg, 1.77 mmol, 1.0 equiv) in anhydrous DMF (10 mL, 10 vols) under a nitrogen atmosphere. The reaction mixture was heated at 60 °C for 2 hours. The mixture was quenched with water and the organic materials were extracted with MTBE three times. The combined organic layers were washed with brine, dried over MgSO₄, and filtered. The volatile materials were removed, and the resulting residue was purified by Biotage Purification to give Compound 135 (259 mg, 34%). ¹H NMR (400 MHz, CHLOROFORM-d) δ ppm 7.89-7.93 (1H, m), 7.81-7.88 (2H, m), 7.77 (1H, s), 7.50-7.56 (2H, m), 7.43 (1H, dd, J=8.50, 1.88 Hz), 7.27-7.35 (5H, m), 6.87 (1H, br d, J=6.88 Hz), 6.60 (1H, d, J=15.88 Hz), 6.24 (1H, dd, J=15.82, 7.19 Hz), 5.15-5.24 (2H, m), 3.80 (1H, dd, J=12.13, 10.01 Hz); LRMS (ESI, m/z) Calculated for [C₂₄H₁₈F₃NO₃+H]⁺ 426.1. Found 426.3.

### Example 51: Making Compound 136

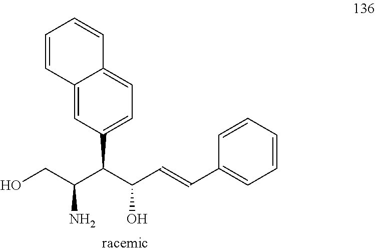 [SMILES: N[C@@H](CO)[C@@H](c1ccc2ccccc2c1)[C@@H](O)/C=C/c1ccccc1]

Compound 136: A solution of Compound 135 (2.6 g, 6.11 mmol, 1.0 equiv) in ethanol (50 mL, 20 vols) was cooled in an ice bath and treated with sodium borohydride (254 mg, 6.72 mmol, 1.1 equiv) portionwise. The mixture was slowly allowed to warm to room temperature and quenched with NaHCO₃ (sat). The mixture was concentrated to half of the original volume and the rest was extracted with ethyl acetate and washed with brine. The combined aqueous phases were back extracted with ethyl acetate and washed with brine. The combined organic phases were concentrated to give the desired amino alcohol, which was used without purification. The above crude mixture was dissolved in 1,4-dioxane (40 ml, 16 vol) and water (10 ml, 4 vol) followed by the addition of lithium hydroxide monohydrate (1.28 g, 30.5 mmol, 5 equiv). The reaction was permitted to stir overnight. The reaction was subsequently quenched by the addition of water and the compound was extracted with EtOAc×3, and dried with brine and MgSO₄. The compound was evaporated to afford crude Compound 136 (1.8 g, 88% yield).

¹H NMR (400 MHz, CHLOROFORM-d) δ ppm 7.87 (1H, s), 7.74-7.85 (4H, m), 7.36-7.56 (4H, m), 7.18-7.26 (5H, m), 6.69 (1H, d, J=15.63 Hz), 6.19 (1H, dd, J=15.82, 5.69 Hz), 4.99 (1H, t, J=5.75 Hz), 3.63-3.78 (5H, m), 3.50-3.59 (1H, m), 3.24 (1H, br dd, J=6.25, 3.75 Hz); LRMS (ESI, m/z) Calculated for [C₂₂H₂₃NO₂+H]⁺ 334.1. Found 334.0.

### Example 52: Making Compound 170

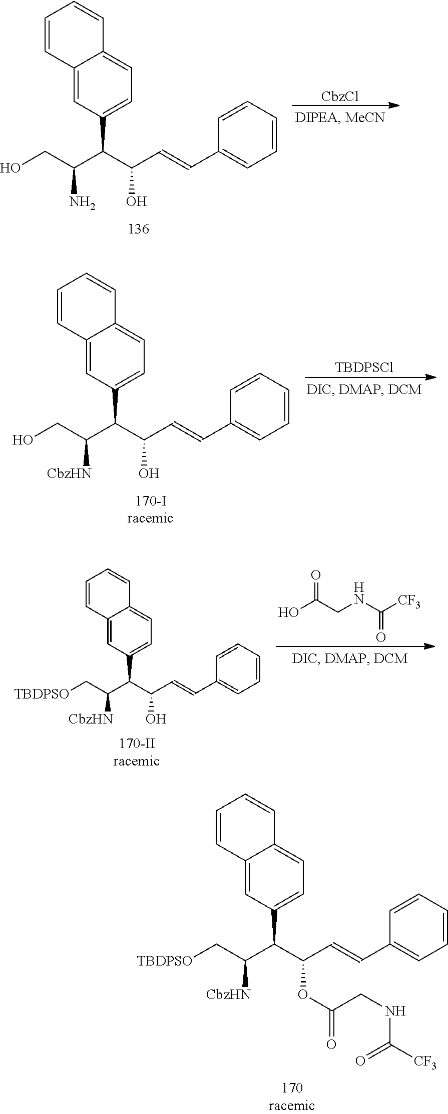

Compound 170: Cbz-Cl (1.1 ml, 7.9 mmol, 1.1 equiv) was added to a suspension of Compound 136 (2.4 g, 7.2 mmol, 1 equiv) in aqueous sodium bicarbonate (29.0 ml, 12 vols) and 1,4-dioxane (48.0 ml, 20 vols). The reaction mixture was stirred at room temperature overnight, whereupon the reaction was complete. The suspension was diluted with EtOAc, and the mixture poured into water. After separation, the aqueous phase was extracted with EtOAc (3×). The organic layer was washed with aqueous NH₄Cl and brine. The combined organic solutions were dried over Na₂SO₄, filtered, and concentrated. The resulting residue was purified by Biotage purification to afford Compound 170-I (2.5 g, 74% yield).

Triethylamine (1.1 ml, 8.0 mmol, 1.5 equiv), DMAP (0.065 g, 0.54 mmol, 0.1 equiv) and TBDPS-Cl (1.5 ml, 5.7 mmol, 1.05 equiv) were added at 0 °C to a solution of Compound 170-1 (2.5 g, 5.3 mmol, 1.0 equiv) in a DCM (25.00 ml, 10 vols) and DMF (25.00 ml, 10 vols) and was permitted to slowly warm to room temperature and stirred for 24 hours. The reaction mixture was diluted with EtOAc and the organic layer was washed with brine. The organic layer was dried over MgSO₄ and was concentrated. The compound was purified via Biotage to Compound 170-II (2.78 g, 74% yield).

A mixture of Compound 170-1 (2.67 g, 3.78 mmol, 1.0 equiv), (2,2,2-trifluoroacetyl)glycine (0.712 g, 4.16 mmol, 1.1 equiv) was taken into DCM (54 ml, 20 vols) to give a slightly yellow suspension. It was cooled in an ice-bath and N,N′-di(propan-2-yl)methanediimine (0.648 ml, 4.16 mmol, 1.1 equiv) was added in portions. The mixture was slowly allowed to RT and stirred overnight. The mixture was taken into EtOAc and subsequently washed with NaHCO₃ (sat)×2, brine, dried over sodium sulfate and concentrated. The residue was purified via Biotage Purification to afford the corresponding Compound 170 (2.4 g, 2.8 mmol, 74% yield)

¹H NMR (400 MHz, CHLOROFORM-d) δ ppm 7.71-7.81 (3H, m), 7.67-7.71 (1H, m), 7.62-7.67 (1H, m), 7.46-7.57 (6H, m), 7.28-7.43 (10H, m), 7.21-7.26 (2H, m), 7.09-7.19 (5H, m), 6.54 (1H, d, J=15.76 Hz), 6.02 (1H, dd, J=10.94, 7.44 Hz), 5.92 (1H, dd, J=15.76, 7.25 Hz), 5.06 (2H, s), 4.50-4.59 (1H, m), 4.43-4.49 (1H, m), 4.35 (1H, dd, J=17.07, 5.19 Hz), 4.16 (1H, dd, J=17.13, 5.38 Hz), 3.60 (1H, dd, J=11.01, 3.13 Hz), 3.27-3.37 (2H, m), 1.05 (9H, s); LRMS (ESI, m/z) Calculated for [C₄₆H₄₆NO₃Si-C₄H₃F₃NO3]⁺ 688.3. Found 688.1.

### Example 53: Making Compound 171

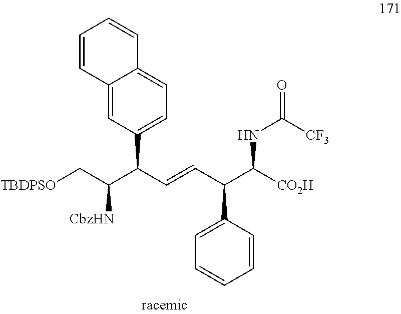

Compound 171: A solution of diisopropylamine (11.18 ml, 11.175 mmol, 4.0 equiv) and n-butyllithium (4.14 ml, 11.175 mmol, 4.0 equiv) in heptane (1.7 M) was added to a solution of zinc chloride (2.206 ml, 4.191 mmol, 1.5 equiv) in 2-Me-THF and Compound 170 (2.4 g, 2.8 mmol, 1.0 equiv), in THF (25.0 ml, 10 vols) at −78 °C. The solution was slowly allowed to warm to room temperature and stirred overnight. The reaction was quenched with 1 M HCl. The mixture was extracted with MTBE twice, dried, and concentrated to afford crude Compound 171 (2.4 g, 100% yield).

¹H NMR (400 MHz, CHLOROFORM-d) δ ppm 7.71-7.86 (3H, m), 7.55-7.66 (6H, m), 7.32-7.50 (10H, m), 7.30 (5H, br d, J=7.63 Hz), 7.21-7.26 (4H, m), 6.56 (1H, br d, J=8.76 Hz), 6.20 (1H, dd, J=14.95, 9.69 Hz), 5.92 (1H, dd, J=15.01, 9.38 Hz), 5.14 (2H, s), 4.91-4.97 (1H, m), 4.65-4.72 (1H, m), 4.22 (1H, br dd, J=10.82, 3.94 Hz), 3.97 (1H, dd, J=9.76, 3.63 Hz), 3.55 (1H, br t, J=9.82 Hz), 3.40 (1H, d, J=6.75 Hz), 1.08 (9H, s); LRMS (ESI, m/z) Calculated for [C₄₈H₄₈NO₃Si+H]⁺ 859.3. Found 859.0.

### Example 54: Making Compound 172

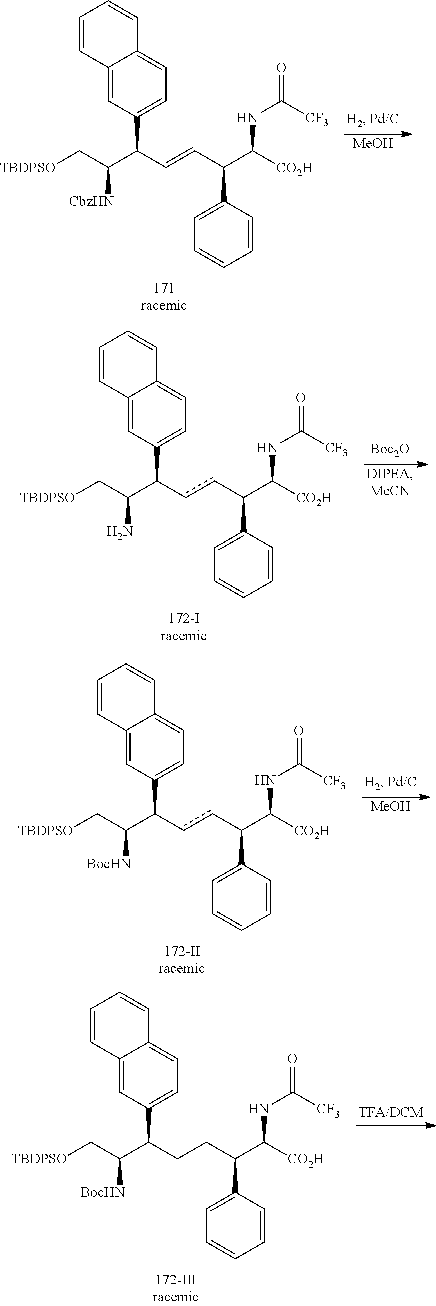

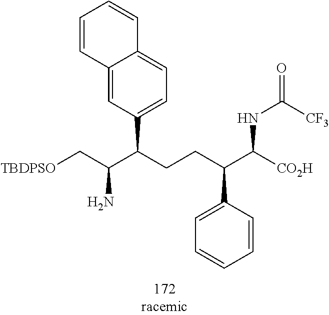

Compound 172: Compound 171 (2.4 g, 2.8 mmol, 1 equiv) was dissolved in methanol (50 ml, 20 vol) to which Pd-C (1.200 g, 10% wt. Pd basis, 50% g/g) was added. The atmosphere was purged with vacuum and backfilled with hydrogen. This cycle was repeated three times and the reaction was stirred overnight. The resulting suspension was filtered through a pad of Celite and the organics were concentrated. The resulting residue was a mixture of the Cbz-deprotected alkene and alkane (Compound 172-I).

Compound 172-I (500 mg, 0.69 mmol, 1 equiv) was dissolved in 1,4-dioxane (20.00 ml, 40 vols) and sat sodium bicarbonate (3 ml, 6 vols) was added. Boc-anhydride (0.208 ml, 0.897 mmol, 1.3 equiv) was added and stirred overnight. The resulting solution was partitioned between EA and water. The aq layer was extracted 3× with EtOAc. The organics were dried with brine and Na₂SO₄ and concentrated to afford Compound 172-II (880 mg).

Compound 172-II (733 mg) was dissolved in methanol (21 mL, 29 vols) to which Pd-C (733 mg, 10% wt. Pd basis, 100% g/g) was added. The atmosphere was purged with vacuum and backfilled with hydrogen. This cycle was repeated three times and the reaction was stirred overnight. The resulting suspension was filtered through a pad of Celite and the organics were concentrated to afford Compound 172-III (735 mg, 31% yield over 3 steps).

Compound 172-III (290 mg, 0.35 mmol, 1.0 equiv) was dissolved in DCM (17 mL, 59 vols) and TFA (3 mL, 10 vol) and permitted to stir 2 h. Upon completion of the reaction, the solvent was removed and the solid was azeotroped with toluene 3×. Compound 172 (300 mg, 98%) was collected as a TFA salt and of sufficient purity to proceed to the next step. LRMS (ESI, m/z) Calculated for [C42H45F3N2O4Si+H]+ 727.3. Found 727.3.

### Example 54: Making Compound 142

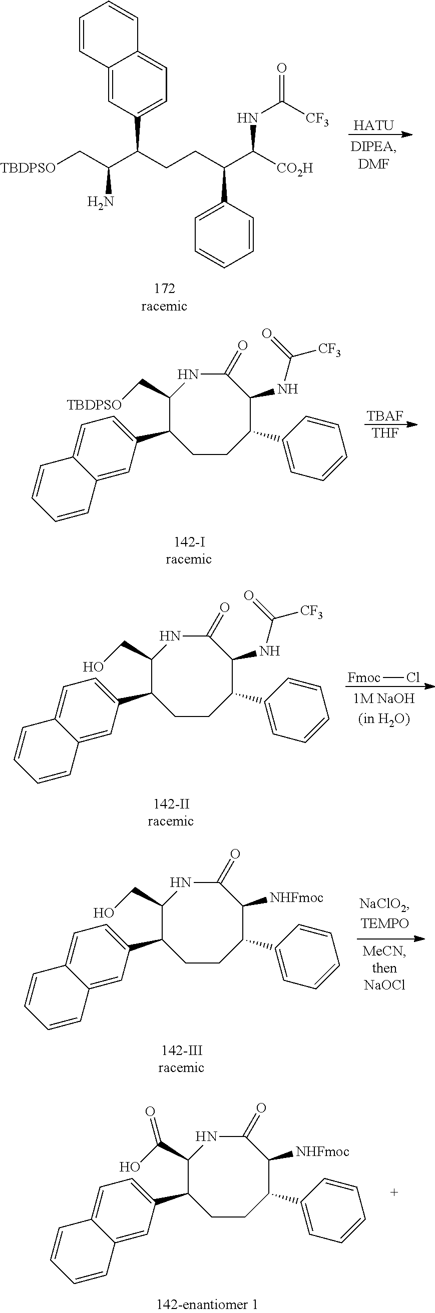

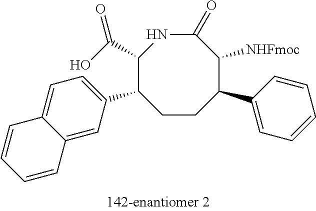

Compound 142: Compound 172 (1 equiv) is dissolved in dry DMA (2400 vols), and HOBt (1.04 equiv), HATU (1.01 equiv), and DIPEA (2.5 equiv) are added sequentially. The solution is permitted to stir for 12 h. The solution is poured into EtOAc and the organic layer is washed with 10% Na₂CO₃ (aq)×3, brine, 1 N HCl (three times), and brine again. The organic layer is dried with MgSO₄, filtered, and concentrated. The substrate is purified by Biotage purification to afford Compound 142-I.

To a solution of Compound 142-I (1 equiv) in THF (20 vols) is added a solution of TBAF (1.5 equiv) (1M in THF). The resulting mixture is stirred at room temperature for 3 h and quenched with a NH₄Cl (sat) solution. The aqueous phase is extracted with EtOAc twice and the combined organic layers are washed with brine, dried over MgSO₄, filtered and concentrated in vacuo. The residue is purified by Biotage purification affording Compound 142-II.

Fmoc-Cl (1.2 equiv) is added to a solution of Compound 142-II (1.0 equiv) in 1 N NaOH (20 vols) at 0 °C. After 24 hours at room temperature, the reaction mixture is diluted with H₂O and the pH is adjusted to 2-3 with 2 N HCl. The aqueous layer is extracted with EtOAc three times. The combined organic phases are washed with brine (1×). After drying over Na2SO4 and filtration, the solvent is removed by rotary evaporation to afford Compound 142-III.

A solution of NaClO2 (80% w/w, 2.8 equiv) in phosphate buffer (0.67 N, pH=7, 10 vols) is added dropwise to a stirred mixture of Compound 142-III (1.0 equiv) and TEMPO (1.50 equiv) in MeCN (15 vols) at 0 °C. NaOCl (6% bleach, 0.7 equiv) is added dropwise to the mixture over 30 minutes. The reaction mixture is allowed to stir at 0 °C for 3 hours. The reaction is quenched with saturated Na₂SO3 and allowed to warm to room temperature. The biphasic solution is acidified with 12 M HCl (pH approximately 2), and the aqueous layer is extracted with DCM three times. The combined organic layer is dried with MgSO₄, filtered, and concentrated. The material is purified via Biotage Purification to afford 142-enantiomer 1 and 142-enantiomer 2.

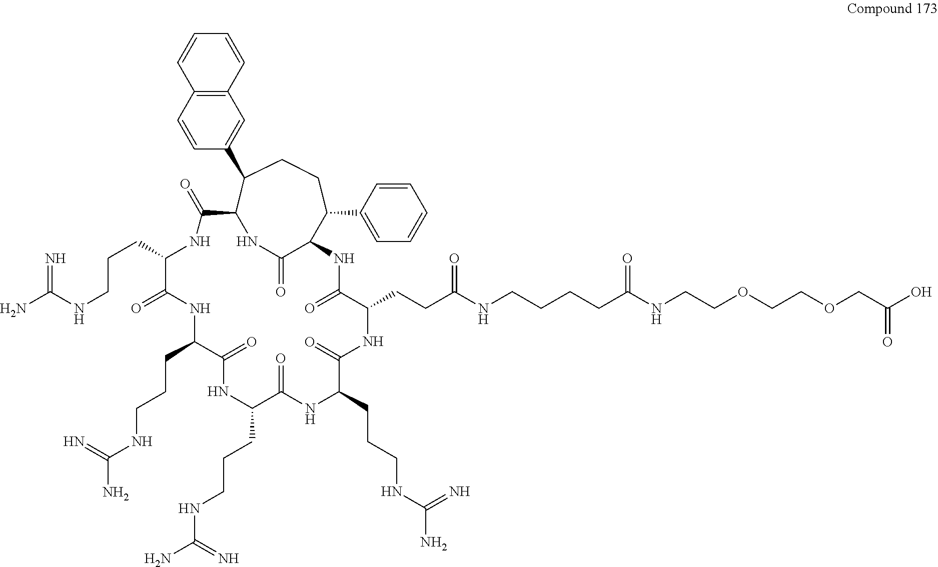 [SMILES: N=C(N)NCCC[C@@H]1NC(=O)[C@@H](CCCNC(=N)N)NC(=O)[C@H](CCCNC(=N)N)NC(=O)[C@@H]2NC(=O)[C@H](NC(=O)[C@H](CCC(=O)NCCCCC(=O)NCCOCCOCC(=O)O)NC(=O)[C@@H](CCCNC(=N)N)NC1=O)[C@@H](c1ccccc1)CC[C@@H]2c1ccc2ccccc2c1]

Compound 173 is prepared by standard Fmoc solid-phase synthesis protocols described in Example 13.

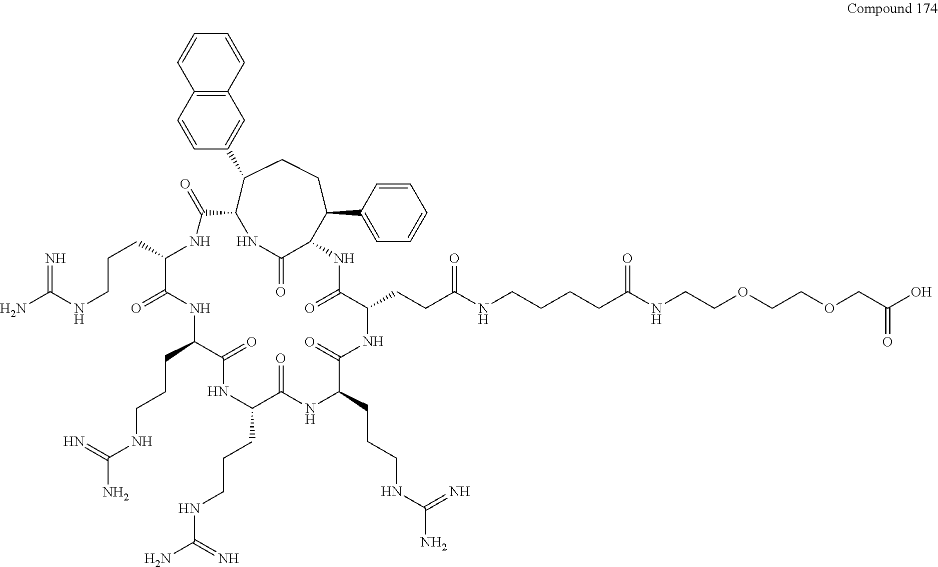 [SMILES: N=C(N)NCCC[C@@H]1NC(=O)[C@H]2NC(=O)[C@@H](NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O)[C@H](c1ccccc1)CC[C@H]2c1ccc2ccccc2c1]

Compound 174 is prepared by standard Fmoc solid-phase synthesis protocols described in Example 13.

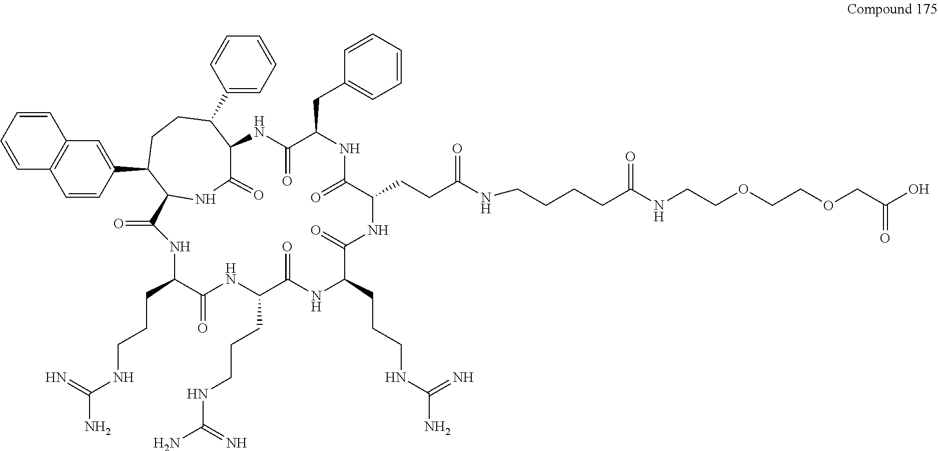 [SMILES: N=C(N)NCCC[C@@H]1NC(=O)[C@@H](CCCNC(=N)N)NC(=O)[C@@H]2NC(=O)[C@H](NC(=O)[C@@H](Cc3ccccc3)NC(=O)[C@H](CCC(=O)NCCCCC(=O)NCCOCCOCC(=O)O)NC(=O)[C@@H](CCCNC(=N)N)NC1=O)[C@@H](c1ccccc1)CC[C@@H]2c1ccc2ccccc2c1]

Compound 175 is prepared by standard Fmoc solid-phase synthesis protocols described in Example 13.

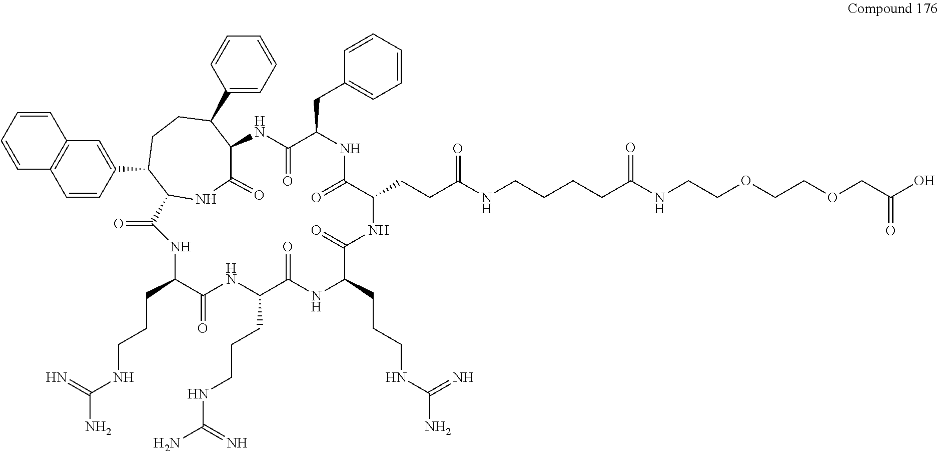 [SMILES: N=C(N)NCCC[C@@H]1NC(=O)[C@@H](CCCNC(=N)N)NC(=O)[C@H]2NC(=O)[C@H](NC(=O)[C@@H](Cc3ccccc3)NC(=O)[C@H](CCC(=O)NCCCCC(=O)NCCOCCOCC(=O)O)NC(=O)[C@@H](CCCNC(=N)N)NC1=O)[C@H](c1ccccc1)CC[C@H]2c1ccc2ccccc2c1]

Compound 176 is prepared by standard Fmoc solid-phase synthesis protocols described in Example 13.

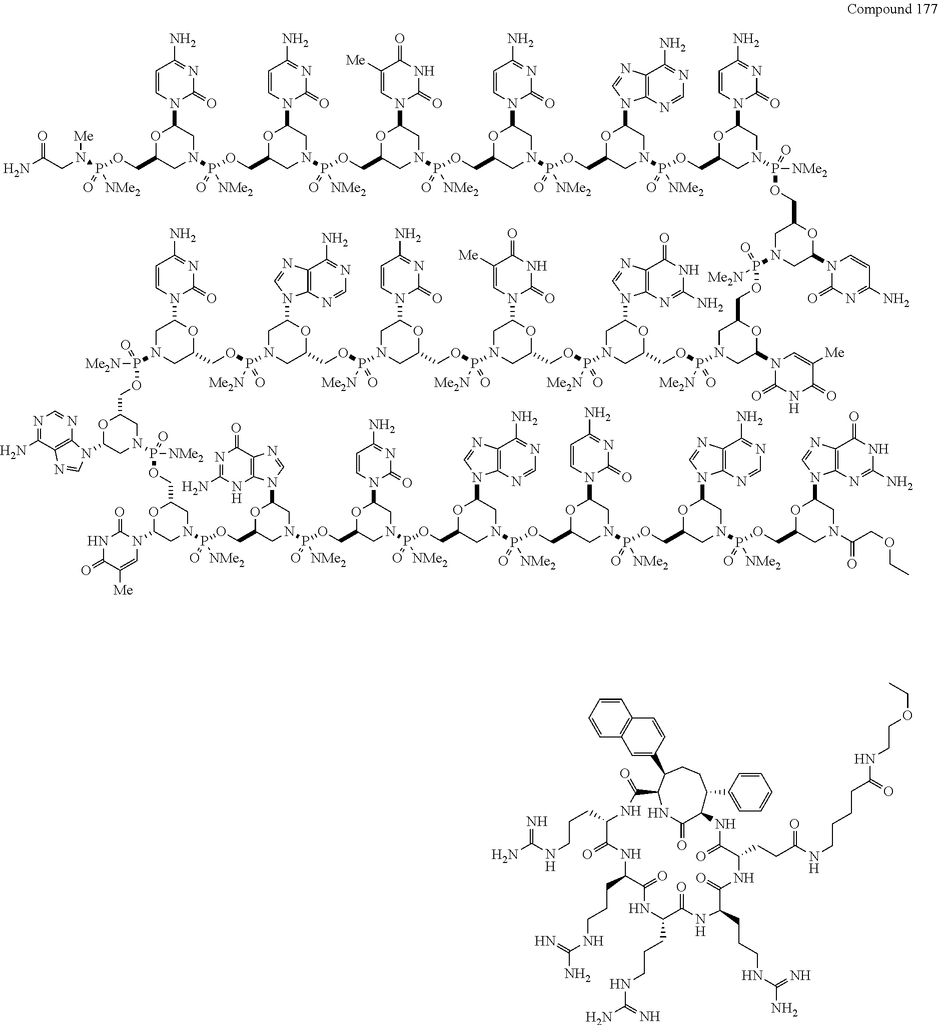

Compound 177 is prepared from PMO-424 and Compound 173 by using General Procedure E.

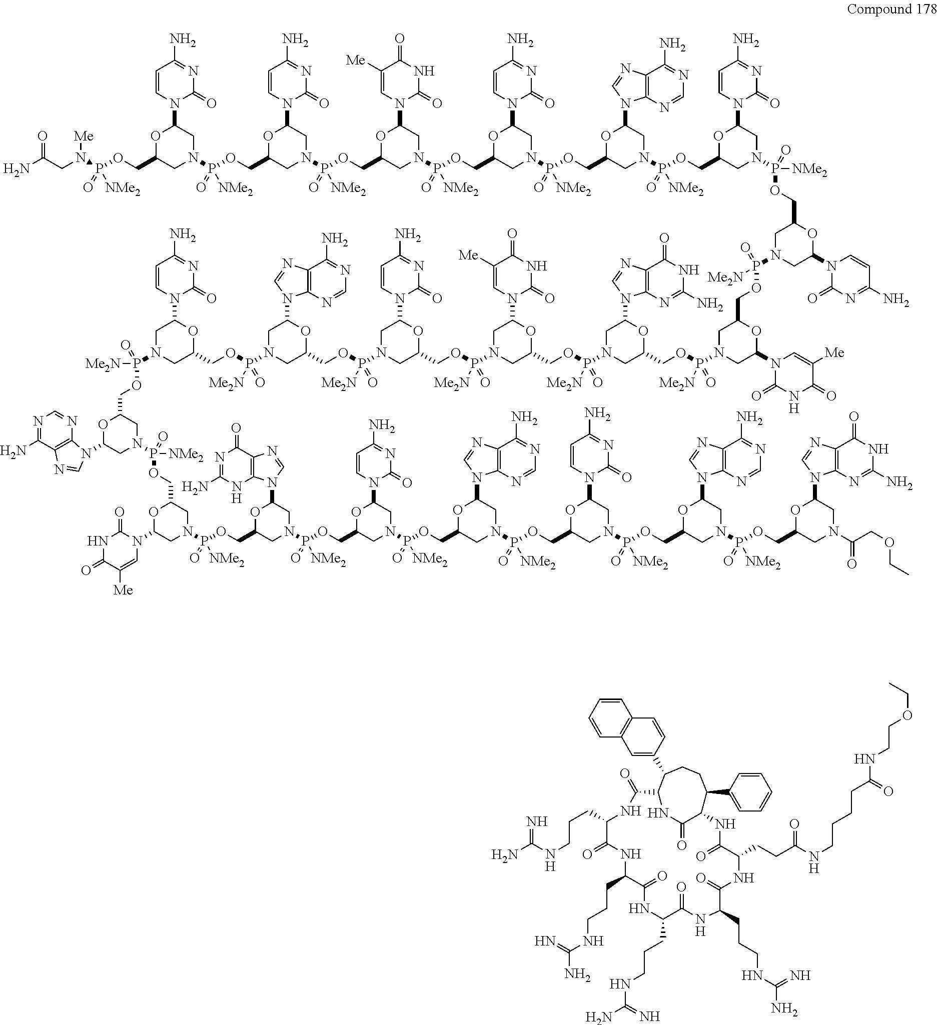

Compound 178 is prepared from PMO-002 and Compound 174 by using General Procedure E.

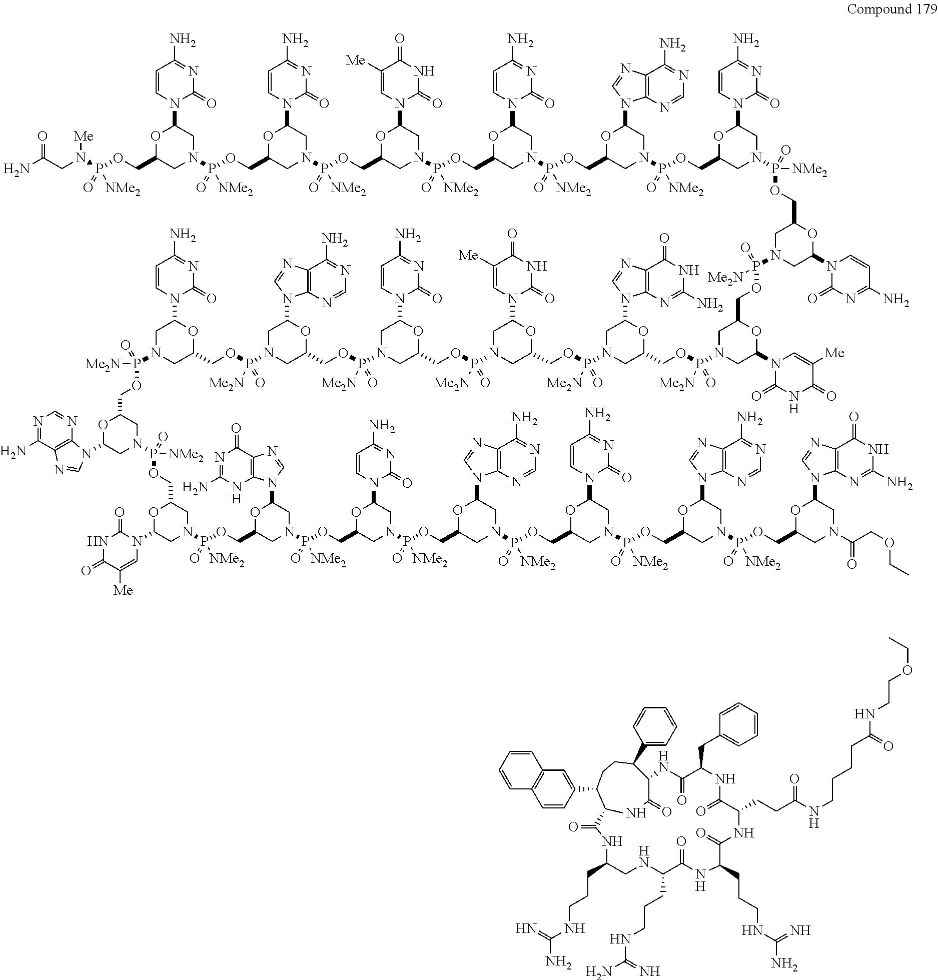

Compound 179 is prepared from PMO-424 and Compound 175 by using General Procedure E.

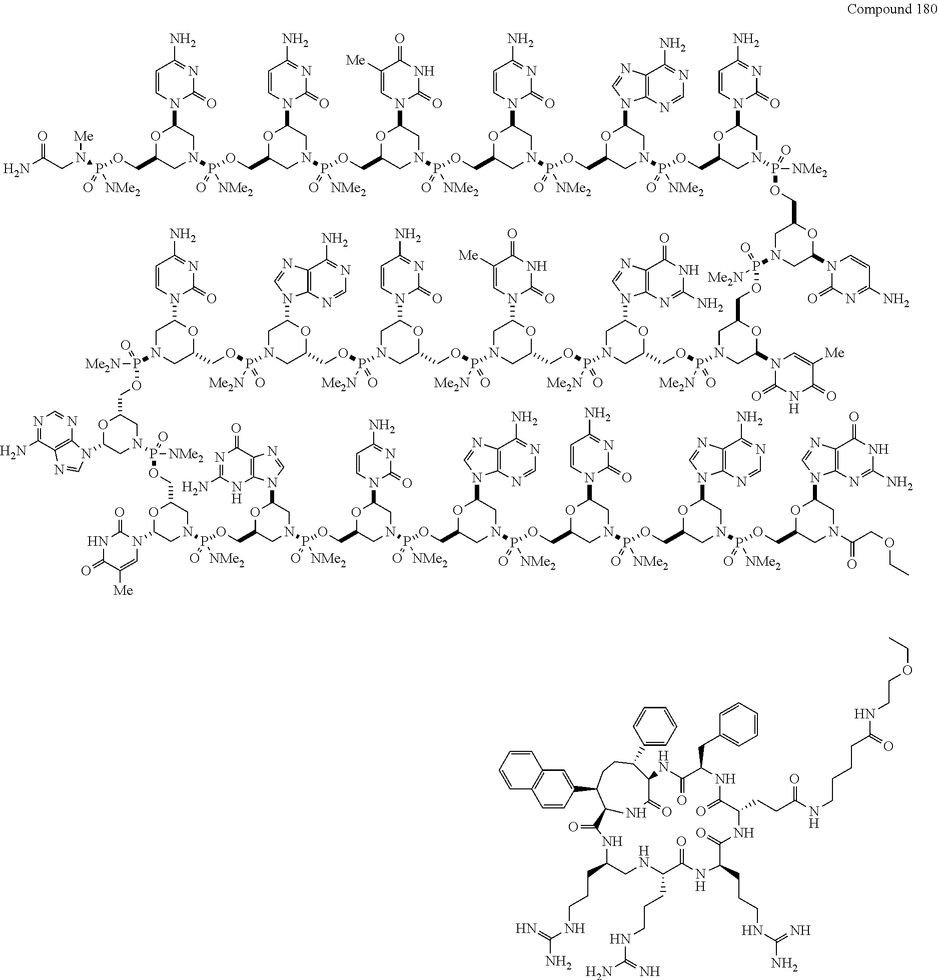

Compound 180 is prepared from PMO-424 and Compound 176 by using General Procedure E.

### Example 55: In Vitro Assays

Assay for cell-penetrating peptide Internalization: A modified assay as the one reported in ACS Chem. Biol. 2013, 8, 423-431 was used to assess cellular uptake of some cell-penetrating peptides. U118MG cells were cultured in DMEM supplemented with 10% FBS and 1% Penicillin-Streptomycin. At approximately 70% confluency, the cells were trypsinized, seeded in 96-well black wall plates, and cultured to 70% confluency. On the day of analysis, each well was washed 3× using PBS. After the third wash, Cell-penetrating peptide 4-MU conjugates (Compound 160, Compound 161, Compound 162) diluted in PBS and controls were added to the appropriate wells. Fluorescence intensity was measured every 5 minutes over a 4-hour period on SPARK plate reader at 37 °C. Each condition was tested along with a compound+DTT control that represents 100% of signal from the released 4-MU fluorophore, a cell only control, and a PBS only control. The values of the replicates were averaged and the uptake % at a given time was calculated using the following equation:

$$Uptake \% = \frac{signal ( CPP + Cells ) - signal ( CPP + PBS )}{signal ( CPP + DTT ) - signal ( CPP + PBS )}$$

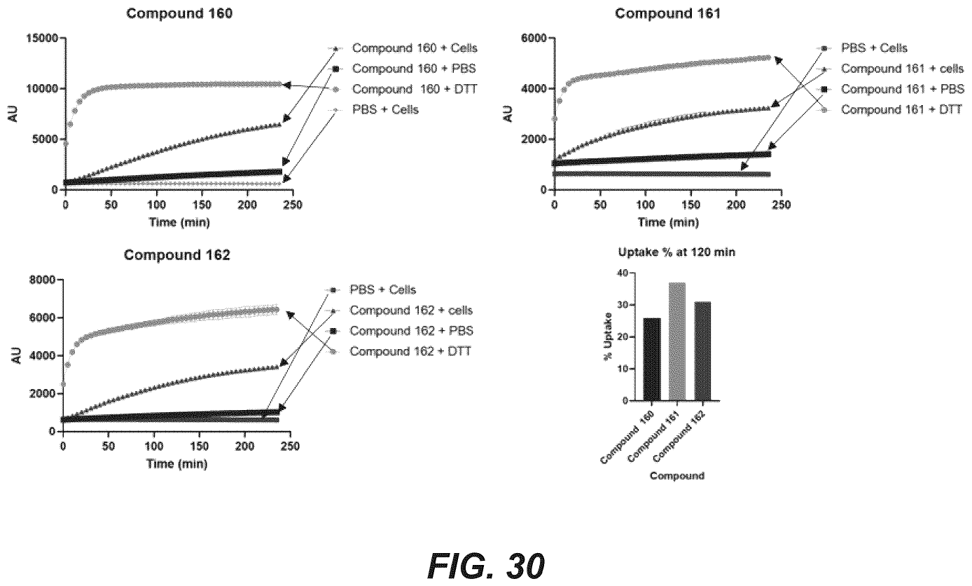

The results of the assay for Compound 160, Compound 161, and Compound 162 are shown in FIG. 30.

### Example 56: In Vivo Studies of Compounds 166/167 and 168/169

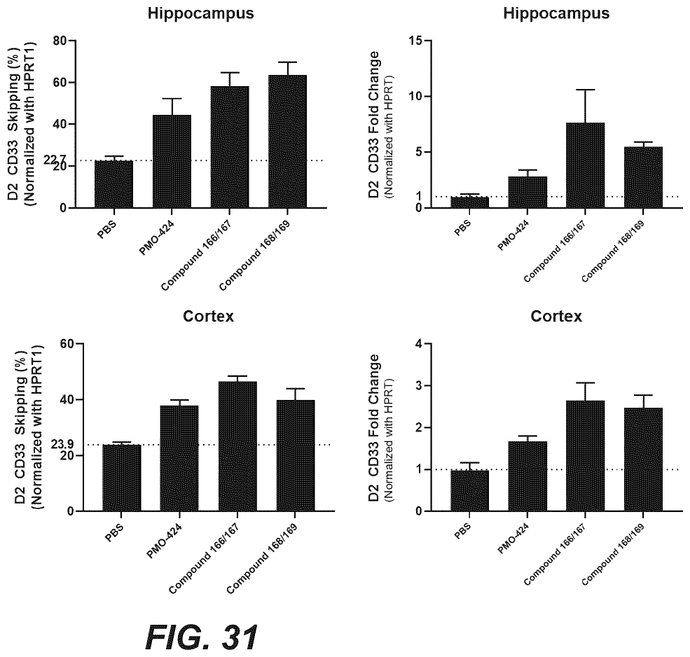

Compound 166/167 and Compound 168/169 were evaluated (as mixtures) in an in-vivo study in the transgenic hCD33 mouse model with a single 30 μg ICV injection and readout after 7-days. The results of qPCR analysis are shown in FIG. 31.

### Example 57: In Vivo Pharmacokinetic (PK)/Pharmacodynamic (PD) Studies of Compounds 31 & 33

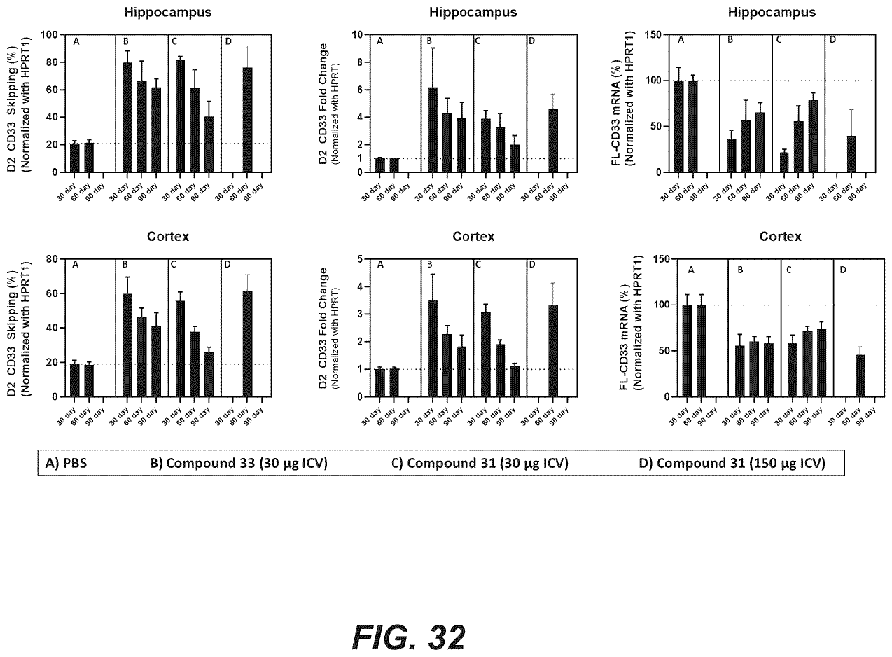

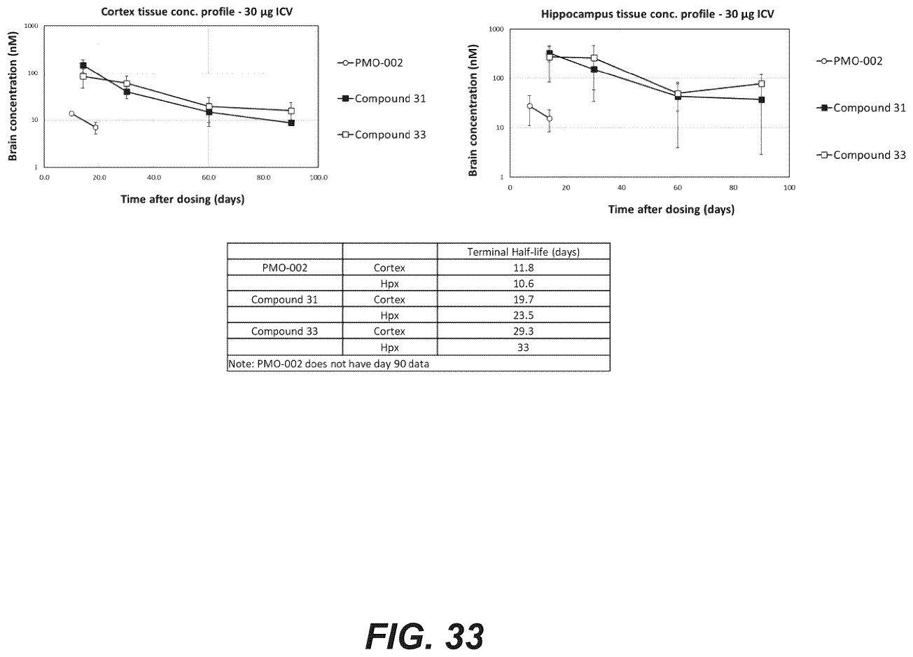

Compound 31 and Compound 33 were evaluated for PK/PD in a 90-day duration study after a single 30 μg dose (and in the case of Compound 31, a 150 μg dose with 60 day readout). Both compounds showed significant PD and PK at 90 days after single administration (FIGS. 32 and 33). The 150 μg dose of Compound 31 (administered as a bolus single injection in 10 μL PBS as vehicle) was tolerated acutely, and did not lead to histopathological changes in the cerebrum of injected animals after 60 days. Both Compound 31 and Compound 33 were tolerated after a single 30 μg ICV dose and did not show any histopathological changes in the brains of animals after 60 days.

### Example 58: In Vivo Pharmacokinetic/Pharmacodynamic Study of Compound 31 in 5×FAD hCD33 Mice

Mouse model generation: 5×FAD hCD33 mice were generated by crossing the humanized CD33 mouse model described in Example 18 with the 5×FAD mouse model (doi: 10.1523/JNEUROSCI.1202-06.2006) The 5×FAD mouse model overexpresses human amyloid beta precursor protein with three familial Alzheimer's disease (FAD) mutations (Swedish (K670N, M671L), Florida (I716V), and London (V717I)) and human presenilin 1 containing two FAD mutations (M146L and L286V).

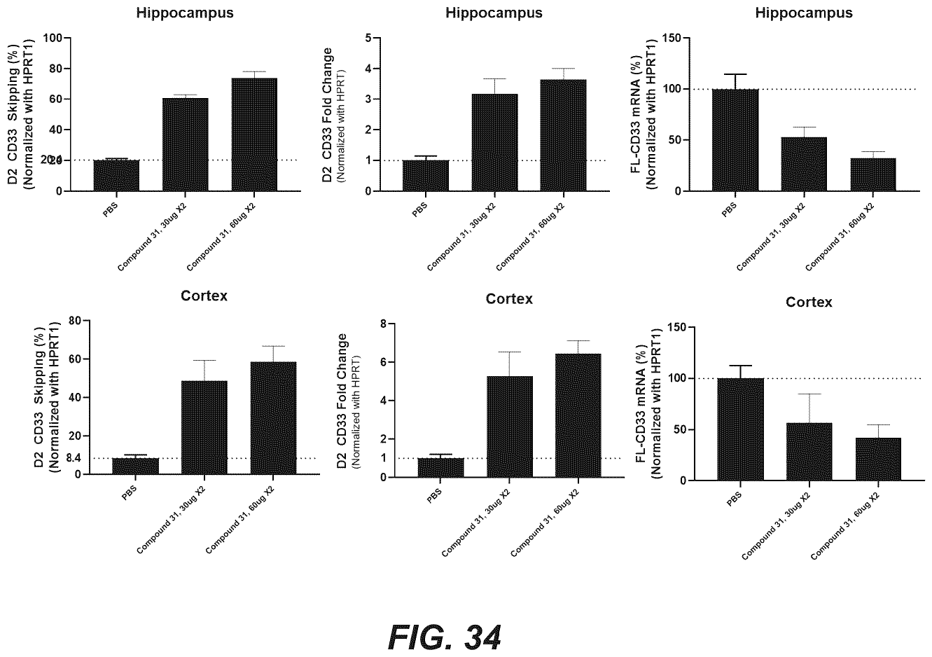

Exon skipping of Compound 31 was also evaluated in the mixed 5×FAD hCD33 mouse model. Two equal doses of Compound 31 were administered ICV at day 0 (30 ug or 60 ug) and again on day 30 (30 ug or 60 ug). Take-down, and qPCR analysis was performed on day 60. The Exon-2 skipping activity of Compound 31 in the hippocampus and cortex in the 5×FAD hCD33 mouse model is shown in FIG. 34.

What is claimed is:

1. A peptide-antisense oligonucleotide conjugate, comprising a cell-penetrating peptide conjugated to an antisense oligonucleotide, wherein the antisense oligonucleotide is complementary to a portion of SEQ ID NO:1, and wherein the peptide-antisense oligonucleotide conjugate has a CD33 Exon-2 skipping efficiency of 30% or greater according to a Standard Exon-Skipping Efficiency Assay for the antisense oligonucleotide.

2. A peptide-antisense oligonucleotide conjugate comprising a cell-penetrating peptide conjugated to an antisense oligonucleotide, wherein the antisense oligonucleotide comprises all or a portion of SEQ ID NO:2, SEQ ID NO:12, or SEQ ID NO:224.

3. The peptide-antisense oligonucleotide conjugate of claim 1 or 2, wherein the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length.

4. The peptide-antisense oligonucleotide conjugate of claim 1 or 2, wherein the antisense oligonucleotide is 21 or 25 nucleotides in length.

5. The peptide-antisense oligonucleotide conjugate of any of claims 1-4, wherein the antisense oligonucleotide comprises one or more non-natural sugar moieties, one or more non-natural internucleotide linkages, or one or more non-natural sugar moieties and one or more non-natural internucleotide linkages.

6. The peptide-antisense oligonucleotide conjugate of claim 5, wherein the antisense comprises one or more non-natural sugar moieties.

7. The peptide-antisense oligonucleotide conjugate of claim 6, wherein the antisense oligonucleotide comprises a phosphorodiamidate morpholino oligomer (PMO).

8. The peptide-antisense oligonucleotide conjugate of claim 7, wherein the antisense oligonucleotide has a CD33 Exon-2 skipping efficiency of 30% or greater according to a Standard Exon-Skipping Efficiency Assay for PMO ASOs.

9. The peptide-antisense oligonucleotide conjugate of claim 6, wherein the antisense oligonucleotide comprises a methoxyethyl ribose oligomer (MOE).

10. The peptide-antisense oligonucleotide conjugate of claim 9, wherein the antisense oligonucleotide has a CD33 Exon-2 skipping efficiency of 30% or greater according to a Standard Exon-Skipping Efficiency Assay for MOE ASOs.

11. The peptide-antisense oligonucleotide conjugate of any of claims 5-10, wherein the antisense oligonucleotide comprises one or more non-natural internucleotide linkages.

12. The peptide-antisense oligonucleotide conjugate of claim 11, wherein the one or more non-natural internucleotide linkages comprise one or more phosphorodiamidate linkages and/or one or more phosphorothioate linkages.

13. The peptide-antisense oligonucleotide conjugate of any of claims 11-12, wherein all of the one or more non-natural internucleotide linkages have an Sp configuration.

14. The peptide-antisense oligonucleotide conjugate of any of claims 11-12, wherein all of the one or more non-natural internucleotide linkages have an Rp configuration.

15. The peptide-antisense oligonucleotide conjugate of any of claims 11-12, wherein the antisense oligonucleotide comprises one or more non-natural internucleotide linkages having an Sp configuration and one or more non-natural internucleotide linkages having an Rp configuration.

16. The peptide-antisense oligonucleotide conjugate of any of claims 1-15, wherein the antisense oligonucleotide comprises one or more modified nucleobases.

17. A composition comprising the peptide-antisense oligonucleotide conjugate of any of claims 1-16 and optionally a pharmaceutically acceptable carrier or excipient.

18. A peptide-antisense oligonucleotide conjugate comprising a cell-penetrating peptide conjugated to all or a portion of PMO-002 (SEQ ID NO:2), MOE-012 (SEQ ID NO:12), or PMO-424 (SEQ ID NO:224).

19. A peptide-antisense oligonucleotide conjugate comprising a cell-penetrating peptide conjugated to an antisense oligonucleotide selected from the group consisting of PMO-002 (SEQ ID NO:2), MOE-012 (SEQ ID NO:12), and PMO-424 (SEQ ID NO:224).

20. The peptide-antisense oligonucleotide conjugate of any of claims 1-19, wherein the peptide comprises at least one proteogenic amino acid, at least one non-proteogenic amino acid, or at least one proteogenic amino acid and at least one non-proteogenic amino acid.

21. The peptide-antisense oligonucleotide conjugate of claim 20, wherein the peptide comprises 5-25 amino acids.

22. The peptide-antisense oligonucleotide conjugate of any of claims 20-21, wherein the non-proteogenic amino acid comprises a modified proline residue, a lipophilic group, and/or a lactam group.

23. The peptide-antisense oligonucleotide conjugate of any of claims 1-22, wherein the peptide is a linear peptide.

24. The peptide-antisense oligonucleotide conjugate of claim 23, wherein the linear peptide comprises at least a portion of Pip6a, ApoE, and/or a neurotensin-based peptide.

25. The peptide-antisense oligonucleotide conjugate of any of claims 1-22, wherein the peptide is a cyclic peptide.

26. The peptide-antisense oligonucleotide conjugate of claim 25, wherein the cyclic peptide is CPP9

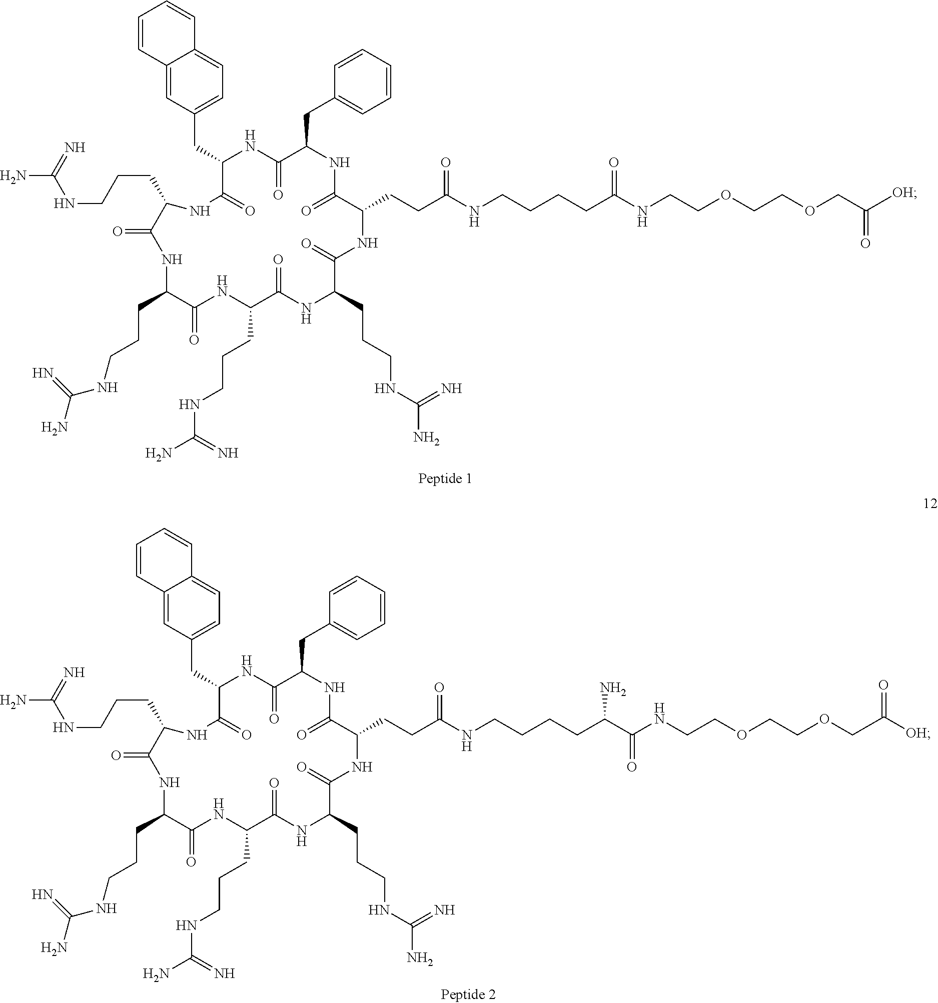 [SMILES: N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O ; N=C(N)NCCC[C@@H]1NC(=O)[C@H](Cc2ccc3ccccc3c2)NC(=O)[C@@H](Cc2ccccc2)NC(=O)[C@H](CCC(=O)NCCCC[C@H](N)C(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O]

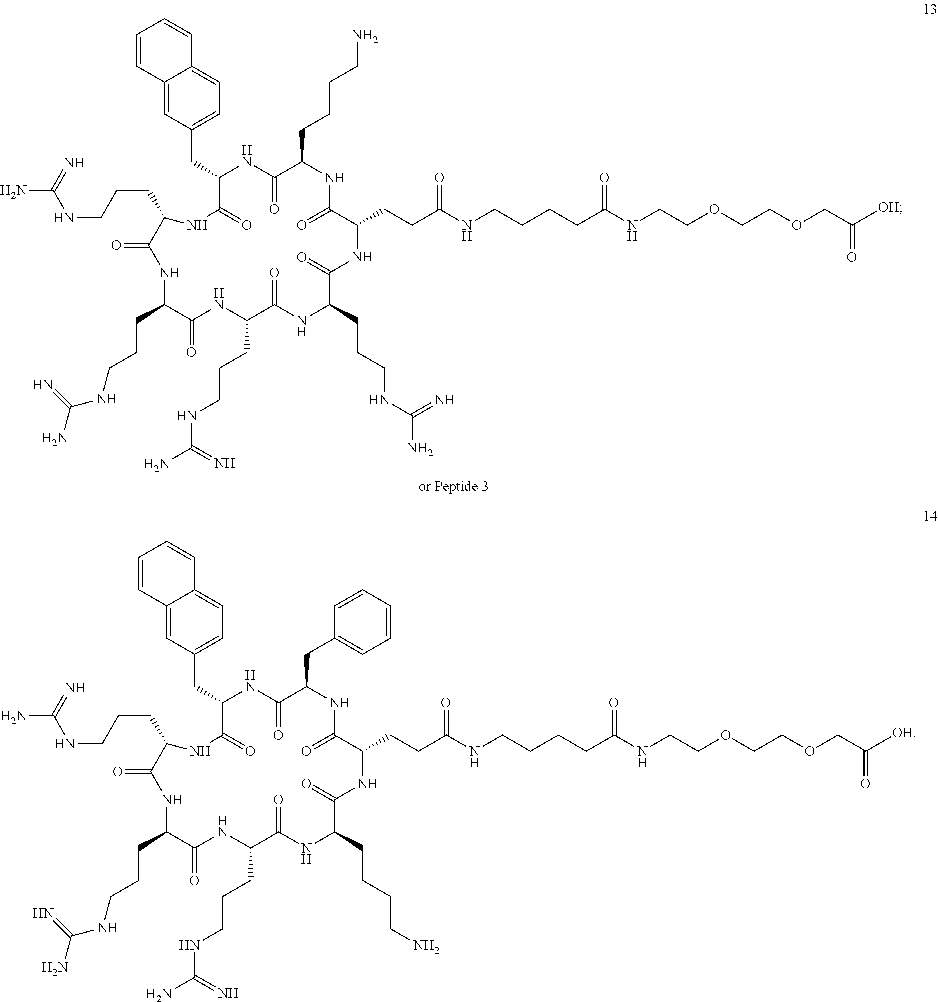 [SMILES: N=C(N)NCCC[C@@H]1NC(=O)[C@H](Cc2ccc3ccccc3c2)NC(=O)[C@@H](CCCCN)NC(=O)[C@H](CCC(=O)NCCCCC(=O)NCCOCCOCC(=O)O)NC(=O)[C@@H](CCCNC(=N)N)NC(=O)[C@H](CCCNC(=N)N)NC(=O)[C@@H](CCCNC(=N)N)NC1=O ; N=C(N)NCCC[C@@H]1NC(=O)[C@H](Cc2ccc3ccccc3c2)NC(=O)[C@@H](Cc2ccccc2)NC(=O)[C@H](CCC(=O)NCCCCC(=O)NCCOCCOCC(=O)O)NC(=O)[C@@H](CCCCN)NC(=O)[C@H](CCCNC(=N)N)NC(=O)[C@@H](CCCNC(=N)N)NC1=O]

27. The peptide-antisense oligonucleotide conjugate of claim 26, wherein the peptide-antisense oligonucleotide conjugate is Compound 31

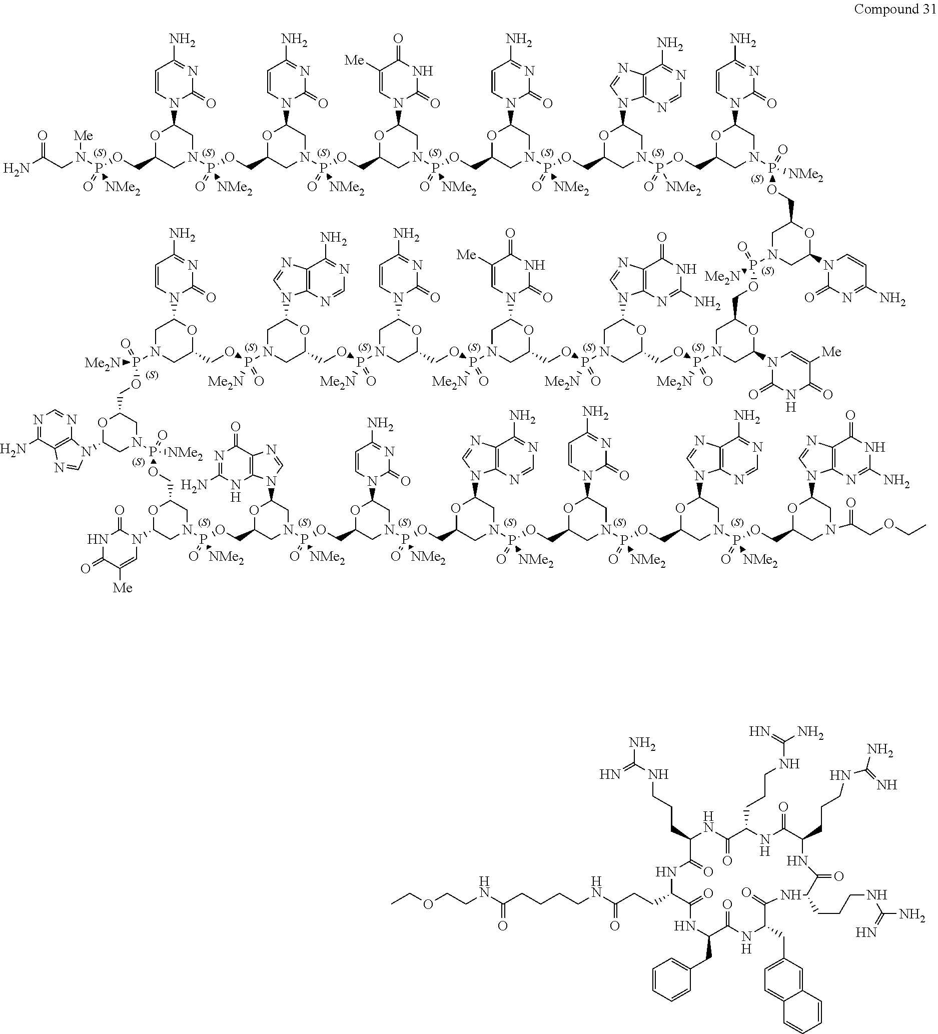

28. The peptide-antisense oligonucleotide conjugate ofany of claims 1-27, wherein the peptide comprises a lipoic acid group.

29. The peptide-antisense oligonucleotide conjugate of claim 28, wherein the peptide is Compound 21

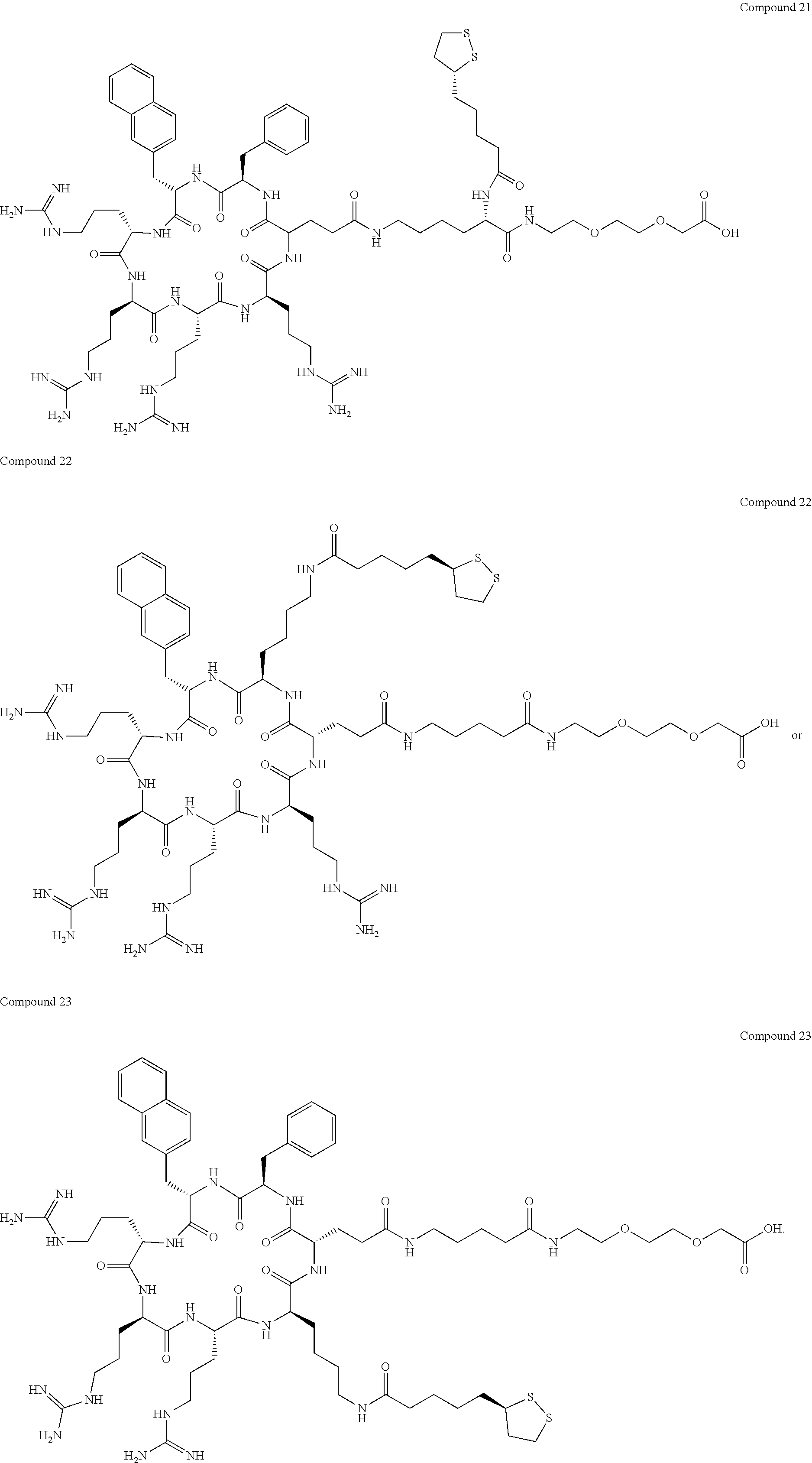

30. The peptide-antisense oligonucleotide conjugate of claim 29, wherein the peptide-antisense oligonucleotide conjugate is Compound 33

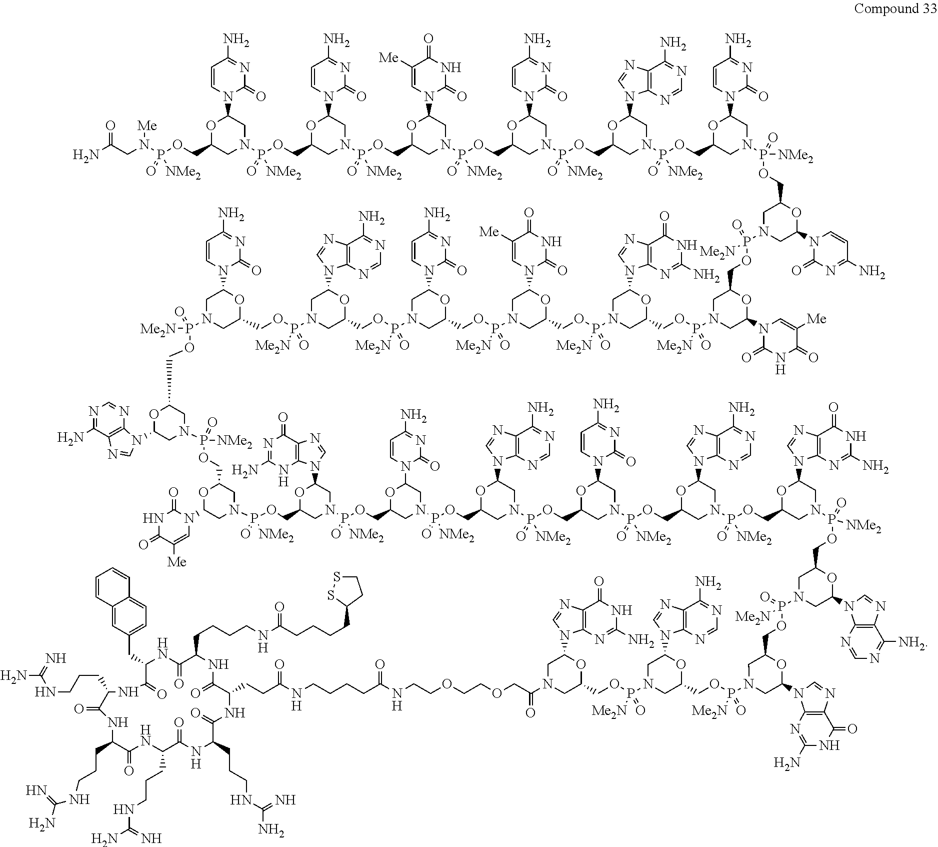

31. The peptide-antisense oligonucleotide conjugate of claim 28, wherein the lipoic acid group is an (R)-lipoic acid group.

32. The peptide-antisense oligonucleotide conjugate of claim 28, wherein the lipoic acid group is an (S)-lipoic acid group.

33. The peptide-antisense oligonucleotide conjugate ofany of claims 1-32, wherein the peptide comprises an oxadiazole linkage.

34. The peptide-antisense oligonucleotide conjugate ofany of claims 1-33, wherein the peptide is conjugated directly to the antisense oligonucleotide.

35. The peptide-antisense oligonucleotide conjugate of claim 34, wherein the peptide is conjugated to the antisense oligonucleotide using a chemical reaction.

36. The peptide-antisense oligonucleotide conjugate of claim 35, wherein the chemical reaction is a strain-promoted azide-alkyne cycloaddition reaction, a strained alkene-tetrazine cycloaddition reaction, or an amide bond reaction.

37. The peptide-antisense oligonucleotide conjugate of any of claims 1-33, wherein the peptide is indirectly conjugated to the antisense oligonucleotide, wherein a linker is conjugated between the peptide and antisense oligonucleotide.

38. The peptide-antisense oligonucleotide conjugate of any of claims 1-37, wherein the peptide-antisense oligonucleotide conjugate further comprises one or more nuclear localization sequences, wherein the one or more nuclear localization sequences are independently conjugated to the peptide and/or the antisense oligonucleotide.

39. A method of inducing Exon-2 skipping in the CD33 gene during pre-mRNA splicing, comprising introducing a peptide-antisense oligonucleotide conjugate into a cell, wherein the peptide-antisense oligonucleotide conjugate comprises a cell-penetrating peptide conjugated to an antisense oligonucleotide, wherein the antisense oligonucleotide is complementary to a portion of SEQ ID NO:1, and wherein the peptide-antisense oligonucleotide conjugate has a CD33 Exon-2 skipping efficiency of 30% or greater according to a Standard Exon-Skipping Efficiency Assay for the antisense oligonucleotide.

40. A method of inducing Exon-2 skipping in the CD33 gene during pre-mRNA splicing, comprising introducing a peptide-antisense oligonucleotide conjugate into a cell, wherein the peptide-antisense oligonucleotide conjugate comprises a cell-penetrating peptide conjugated to an antisense oligonucleotide, and wherein the antisense oligonucleotide comprises all or a portion of SEQ ID NO:2, SEQ ID NO:12, or SEQ ID NO:224.

41. The method of claim 39 or 40, wherein the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length.

42. The method of claim 39 or 40, wherein the antisense oligonucleotide is 21 or 25 nucleotides in length.

43. The method of any of claims 39-42, wherein the antisense oligonucleotide comprises one or more non-natural sugar moieties, one or more non-natural internucleotide linkages, or one or more non-natural sugar moieties and one or more non-natural internucleotide linkages.

44. The method of claim 43, wherein the antisense oligonucleotide comprises one or more non-natural sugar moieties.

45. The method of claim 44, wherein the antisense oligonucleotide comprises a phosphorodiamidate morpholino oligomer (PMO).

46. The method of claim 45, wherein the peptide-antisense oligonucleotide conjugate has a CD33 Exon-2 skipping efficiency of 30% or greater according to a Standard Exon-Skipping Efficiency Assay for PMO ASOs.

47. The method of claim 44, wherein the antisense oligonucleotide comprises a methoxyethyl ribose oligomer (MOE).

48. The method of claim 47, wherein the antisense oligonucleotide has a CD33 Exon-2 skipping efficiency of 30% or greater according to a Standard Exon-Skipping Efficiency Assay for MOE ASOs.

49. The method of any of claims 43-48, wherein the antisense oligonucleotide comprises one or more non-natural internucleotide linkages.

50. The method of claim 49, wherein all of the one or more non-natural internucleotide linkages have an Sp configuration.

51. The method of claim 49, wherein all of the one or more non-natural internucleotide linkages have an Rp configuration.

52. The method of claim 49, wherein the one or more non-natural internucleotide linkages comprise one or more non-natural internucleotide linkages having an Sp configuration and one or more non-natural internucleotide linkages having an Rp configuration.

53. The method of any of claims 39-52, wherein the antisense oligonucleotide comprises one or more modified nucleobases.

54. The method of any of claims 39-53, wherein the peptide-antisense oligonucleotide conjugate further comprises a pharmaceutically acceptable carrier or excipient.

55. A method of inducing Exon-2 skipping in the CD33 gene during pre-mRNA splicing, comprising introducing a peptide-antisense oligonucleotide conjugate into a cell, wherein the peptide-antisense oligonucleotide conjugate comprises an antisense oligonucleotide conjugated to a cell-penetrating peptide, and wherein the antisense oligonucleotide comprises all or a portion of PMO-002 (SEQ ID NO:2), MOE-012 (SEQ ID NO:12) or PMO-424 (SEQ ID NO:224).

56. A method of inducing Exon-2 skipping in the CD33 gene during pre-mRNA splicing, comprising introducing a peptide-antisense oligonucleotide conjugate into a cell, wherein the peptide-antisense oligonucleotide conjugate comprises an antisense oligonucleotide conjugated to a cell-penetrating peptide, and wherein the antisense oligonucleotide is selected from the group consisting of PMO-002 (SEQ ID NO:2), MOE-012 (SEQ ID NO:12) and PMO-424 (SEQ ID NO:224).

57. The method of any of claims 39-56, wherein the cell is an animal cell.

58. The method of claim 57, wherein the cell is a human cell.

59. A method of treating a subject having a neurodegenerative disease comprising administering to said subject a therapeutically effective amount of a peptide-antisense oligonucleotide conjugate, wherein the peptide-antisense oligonucleotide conjugate comprises an antisense nucleotide conjugated to a cell-penetrating peptide, wherein the antisense oligonucleotide is complementary to a portion of SEQ ID NO:1, and wherein the peptide-antisense oligonucleotide conjugate has a CD33 Exon-2 skipping efficiency of 30% or greater according to a Standard Exon-Skipping Efficiency Assay for the antisense oligonucleotide.

60. A method of treating a subject having a neurodegenerative disease comprising administering to said subject a therapeutically effective amount of a peptide-antisense oligonucleotide conjugate, wherein the peptide-antisense oligonucleotide conjugate comprises an antisense oligonucleotide conjugated to a cell-penetrating peptide, and wherein the antisense oligonucleotide is complementary to all or a portion of SEQ ID NO:2, SEQ ID NO:12 or SEQ ID NO:224.

61. The method of any of claims 59-60, wherein the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length.

62. The method of any of claims 59-61, wherein the antisense oligonucleotide is 21 or 25 nucleotides in length.

63. The method of any of claims 59-62, wherein the antisense oligonucleotide comprises one or more non-natural sugar moieties, one or more non-natural internucleotide linkages, or one or more non-natural sugar moieties and one or more non-natural internucleotide linkages.

64. The method of claim 63, wherein the antisense oligonucleotide comprises one or more non-natural sugar moieties.

65. The method of claim 64, wherein the antisense oligonucleotide comprises a phosphorodiamidate morpholino oligomer (PMO).

66. The method of claim 65, wherein the peptide-antisense oligonucleotide conjugate has a CD33 Exon-2 skipping efficiency of 30% or greater according to a Standard Exon-Skipping Efficiency Assay for PMO ASOs.

67. The method of claim 64, wherein the antisense oligonucleotide comprises a methoxyethyl ribose oligomer (MOE).

68. The method of claim 67, wherein the antisense oligonucleotide has a CD33 Exon-2 skipping efficiency of 30% or greater according to a Standard Exon-Skipping Efficiency Assay for MOE ASOs.

69. The method of any of claims 63-68, wherein the antisense oligonucleotide comprises one or more non-natural internucleotide linkages.

70. The method of claim 69, wherein all of the one or more non-natural internucleotide linkages have an Sp configuration.

71. The method of claim 69, wherein all of the one or more non-natural internucleotide linkages have an Rp configuration.

72. The method of claim 69, wherein the one or more non-natural internucleotide linkages comprise one or more non-natural internucleotide linkages having an Sp configuration and one or more non-natural internucleotide linkages having an Rp configuration.

73. The method of any of claims 59-72, wherein the antisense oligonucleotide comprises one or more modified nucleobases.

74. The method of any of claims 59-73, wherein the peptide-antisense oligonucleotide conjugate further comprises a pharmaceutically acceptable carrier or excipient.

75. A method of treating a subject having a neurodegenerative disease comprising administering to said subject a therapeutically effective amount of a peptide-antisense oligonucleotide conjugate, wherein the peptide-antisense oligonucleotide conjugate comprises an antisense oligonucleotide conjugated to a cell-penetrating peptide, and wherein the antisense oligonucleotide comprises all or a portion of PMO-002 (SEQ ID NO:2), MOE-012 (SEQ ID NO:12), or PMO-424 (SEQ ID NO:224).

76. A method of treating a subject having a neurodegenerative disease comprising administering to said subject a therapeutically effective amount of a peptide-antisense oligonucleotide conjugate, wherein the peptide-antisense oligonucleotide conjugate comprises an antisense oligonucleotide conjugated to a cell-penetrating peptide, and wherein the antisense oligonucleotide is selected from the group consisting of PMO-002 (SEQ ID NO:2), MOE-012 (SEQ ID NO:12), or PMO-424 (SEQ ID NO:224).

77. The method of any of claims 59-76, wherein the subject is a human subject.

78. The method of any of claims 59-77, wherein the neurodegenerative disease is Alzheimer's Disease.

79. A peptide-antisense oligonucleotide conjugate according to claim 1 for use in a method of inducing Exon-2 skipping in the CD33 gene during pre-mRNA splicing, comprising introducing into a cell the peptide-antisense oligonucleotide conjugate of claim 1, wherein the peptide-antisense oligonucleotide conjugate hybridizes to a target region of the CD33 gene and induces Exon-2 skipping during pre-mRNA splicing of the CD33 gene.

80. A peptide-antisense oligonucleotide conjugate according to claim 2 for use in a method of inducing Exon-2 skipping in the CD33 gene during pre-mRNA splicing, comprising introducing into a cell the peptide-antisense oligonucleotide conjugate of claim 2, wherein the peptide-antisense oligonucleotide conjugate hybridizes to a target region of the CD33 gene and induces Exon-2 skipping during pre-mRNA splicing of the CD33 gene.

81. The peptide-antisense oligonucleotide conjugate of claim 79 or 80, wherein the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length.

82. The peptide-antisense oligonucleotide conjugate of claim 79 or 80, wherein the antisense oligonucleotide is 21 or 25 nucleotides in length.

83. The peptide-antisense oligonucleotide conjugate of any of claims 79-82, wherein the antisense oligonucleotide comprises one or more non-natural sugar moieties, one or more non-natural internucleotide linkages, or one or more non-natural sugar moieties and one or more non-natural internucleotide linkages.

84. The peptide-antisense oligonucleotide conjugate of claim 83, wherein the antisense oligonucleotide comprises one or more modified sugar moieties.

85. The peptide-antisense oligonucleotide conjugate of claim 84, wherein the antisense oligonucleotide comprises a phosphorodiamidate morpholino oligomer (PMO).

86. The peptide-antisense oligonucleotide conjugate of claim 85, wherein the peptide-antisense oligonucleotide conjugate has a CD33 Exon-2 skipping efficiency of 30% or greater according to a Standard Exon-Skipping Efficiency Assay for PMO ASOs.

87. The peptide-antisense oligonucleotide conjugate of claim 84, wherein the antisense oligonucleotide comprises a methoxyethyl ribose oligomer (MOE).

88. The peptide-antisense oligonucleotide conjugate of claim 87, wherein the antisense oligonucleotide has a CD33 Exon-2 skipping efficiency of 30% or greater according to a Standard Exon-Skipping Efficiency Assay for MOE ASOs.

89. The peptide-antisense oligonucleotide conjugate of any of claims 83-88, wherein the antisense oligonucleotide comprises one or more non-natural internucleotide linkages.

90. The peptide-antisense oligonucleotide conjugate of claim 89, wherein all of the one or more non-natural internucleotide linkages have an Sp configuration.

91. The peptide-antisense oligonucleotide conjugate of claim 89, wherein all of the one or more non-natural internucleotide linkages have an Rp configuration.

92. The peptide-antisense oligonucleotide conjugate of claim 89, wherein the one or more non-natural internucleotide linkages comprise one or more non-natural internucleotide linkages having an Sp configuration and one or more non-natural internucleotide linkages having an Rp configuration.

93. The peptide-antisense oligonucleotide conjugate of any of claims 79-92, wherein the antisense oligonucleotide comprises one or more modified nucleobases.

94. The peptide-antisense oligonucleotide conjugate of any of claims 79-93, wherein the peptide-antisense oligonucleotide conjugate further comprises a pharmaceutically acceptable carrier or excipient.

95. A peptide-antisense oligonucleotide conjugate according to claim 18 for use in a method of inducing Exon-2 skipping in the CD33 gene during pre-mRNA splicing, comprising introducing into a cell the peptide-antisense oligonucleotide conjugate of claim 18, wherein the peptide-antisense oligonucleotide conjugate hybridizes to a target region of the CD33 gene and induces Exon-2 skipping during pre-mRNA splicing of the CD33 gene.

96. A peptide-antisense oligonucleotide conjugate according to claim 19 for use in a method of inducing Exon-2 skipping in the CD33 gene during pre-mRNA splicing, comprising introducing into a cell the peptide-antisense oligonucleotide conjugate of claim 19, wherein the peptide-antisense oligonucleotide conjugate hybridizes to a target region of the CD33 gene and induces Exon-2 skipping during pre-mRNA splicing of the CD33 gene.

97. The peptide-antisense oligonucleotide conjugate of any of claims 79-96, wherein the cell is an animal cell.

98. The peptide-antisense oligonucleotide conjugate of claim 97, wherein the cell is a human cell.

99. A peptide-antisense oligonucleotide conjugate according to claim 1 for use in a method of treating a subject having a neurodegenerative disease, comprising administering to said subject a therapeutically effective amount of the peptide-antisense oligonucleotide conjugate of claim 1.

100. A peptide-antisense oligonucleotide conjugate according to claim 2 for use in a method of treating a subject having a neurodegenerative disease, comprising administering to said subject a therapeutically effective amount of the peptide-antisense oligonucleotide conjugate of claim 2.

101. The peptide-antisense oligonucleotide conjugate of claim 99 or 100, wherein the antisense oligonucleotide is 16-30 nucleotides in length, 18-30 nucleotides in length, 18-25 nucleotides in length, 18-21 nucleotides in length, 21-30 nucleotides in length, 21-25 nucleotides in length, or 25-30 nucleotides in length.

102. The peptide-antisense oligonucleotide conjugate of claim 99 or 100, wherein the antisense oligonucleotide is 21 or 25 nucleotides in length.

103. The peptide-antisense oligonucleotide conjugate of any of claims 99-102, wherein the antisense oligonucleotide comprises one or more non-natural sugar moieties, one or more non-natural internucleotide linkages, or one or more non-natural sugar moieties and one or more non-natural internucleotide linkages.

104. The peptide-antisense oligonucleotide conjugate of claim 103, wherein the antisense oligonucleotide comprises one or more non-natural sugar moieties.

105. The peptide-antisense oligonucleotide conjugate of claim 104, wherein the antisense oligonucleotide comprises a phosphorodiamidate morpholino oligomer (PMO).

106. The peptide-antisense oligonucleotide conjugate of claim 105, wherein the peptide-antisense oligonucleotide conjugate has a CD33 Exon-2 skipping efficiency of 30% or greater according to a Standard Exon-Skipping Efficiency Assay for PMO ASOs.

107. The peptide-antisense oligonucleotide conjugate of claim 104, wherein the antisense oligonucleotide comprises a methoxyethyl ribose oligomer (MOE).

108. The peptide-antisense oligonucleotide conjugate of claim 107, wherein the antisense oligonucleotide has a CD33 Exon-2 skipping efficiency of 30% or greater according to a Standard Exon-Skipping Efficiency Assay for MOE ASOs.

109. The peptide-antisense oligonucleotide conjugate of claim 103-108, wherein the antisense oligonucleotide comprises one or more non-natural internucleotide linkages.

110. The peptide-antisense oligonucleotide conjugate of claim 109, wherein all of the one or more non-natural internucleotide linkages have an Sp configuration.

111. The peptide-antisense oligonucleotide conjugate of claim 109, wherein all of the one or more non-natural internucleotide linkages have an Rp configuration.

112. The peptide-antisense oligonucleotide conjugate of claim 109, wherein the one or more non-natural internucleotide linkages comprise one or more non-natural internucleotide linkages having an Sp configuration and one or more non-natural internucleotide linkages having an Rp configuration.

113. The peptide-antisense oligonucleotide conjugate of any of claims 99-112, wherein the antisense oligonucleotide comprises one or more modified nucleobases.

114. The peptide-antisense oligonucleotide conjugate of any of claims 99-113, wherein the peptide-antisense oligonucleotide conjugate further comprises a pharmaceutically acceptable carrier or excipient.

115. A peptide-antisense oligonucleotide conjugate according to claim 18 for use in a method of treating a subject having a neurodegenerative disease, comprising administering to said subject a therapeutically effective amount of the peptide-antisense oligonucleotide conjugate of claim 18.

116. A peptide-antisense oligonucleotide conjugate according to claim 19 for use in a method of treating a subject having a neurodegenerative disease, comprising administering to said subject a therapeutically effective amount of the peptide-antisense oligonucleotide conjugate of claim 19.

117. The peptide-antisense oligonucleotide conjugate of any of claims 99-116, wherein the neurodegenerative disease is Alzheimer's Disease.

118. A peptide comprising a cyclic peptide comprising a lipoic acid group.

119. The peptide of claim 118, wherein the lipoic acid group is an (R)-lipoic acid group.

120. The peptide of claim 118, wherein the lipoic acid group is an (S)-lipoic acid group.

121. The peptide of any of claims 118-120, wherein the cyclic peptide comprises 4 to 40 amino acids, optionally wherein the cyclic peptide comprises 6 to 10 amino acids.

122. The peptide of any of claims 118-121, wherein the cyclic peptide comprises 1 to 5 arginine residues, optionally wherein the cyclic peptide comprises 2 to 4 arginine residues.

123. The peptide of any of claims 118-122, wherein the cyclic peptide comprises 1-5 aromatic hydrophobic amino acids, optionally wherein the cyclic peptide comprises 2-4 aromatic hydrophobic amino acids.

124. The peptide of any of claims 118-123, wherein the cyclic peptide is chosen from:

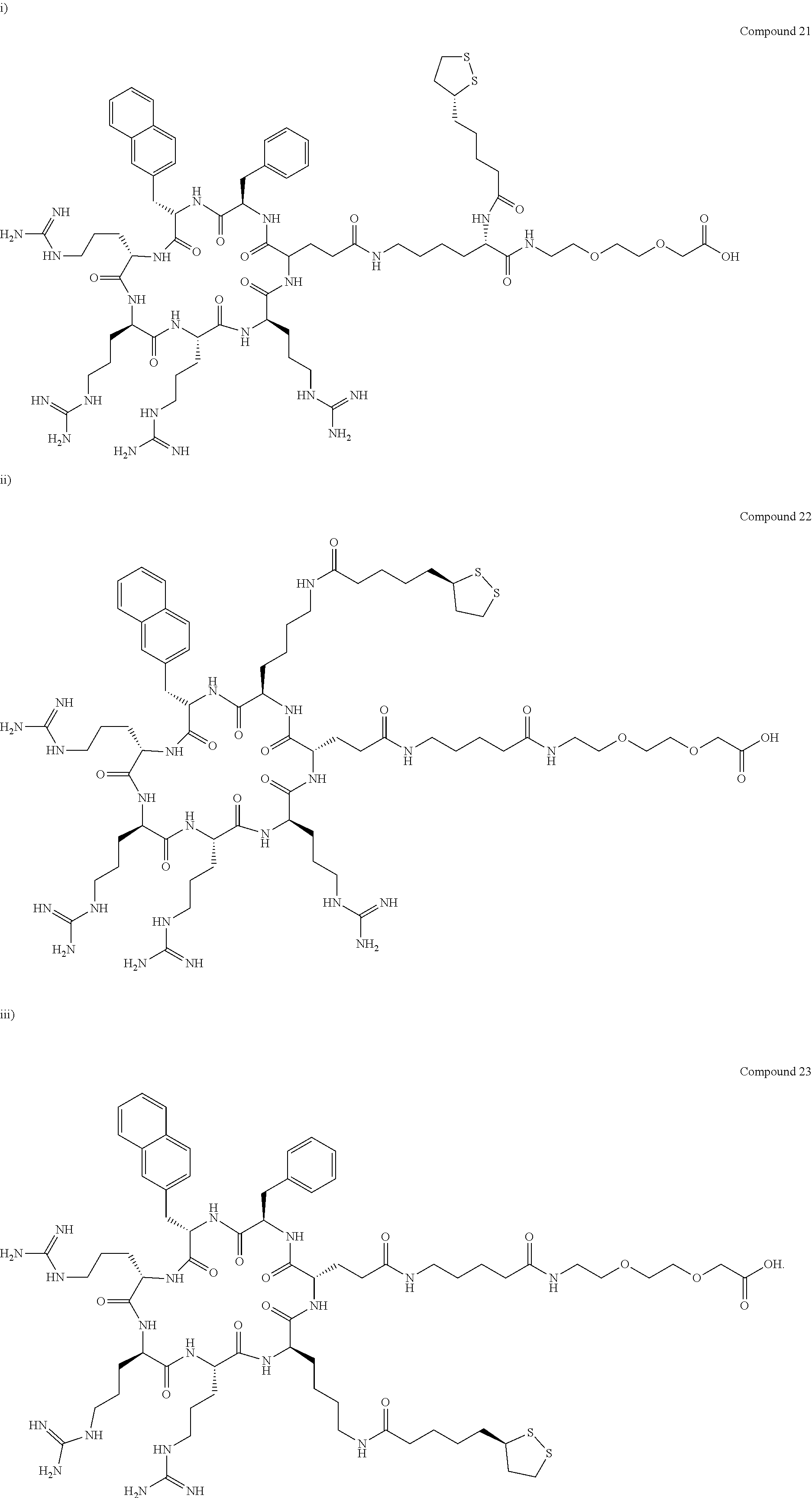

125. The peptide of any of claims 118-124, wherein the cyclic peptide comprises two or more lipoic acid groups.

126. The peptide of claim 125, wherein the two or more lipoic acid groups comprise two or more (R)-lipoic acid groups, two or more (S)-lipoic acid groups, and/or one or more (R)-lipoic acid groups and one or more (S)-lipoic acid groups.

127. A method of making the peptide of claim 124, wherein the peptide is synthesized according to General Procedure C.

128. A peptide comprising a cyclic lactam group.

129. The peptide of claim 128, wherein the peptide is a cell-penetrating peptide.

130. The peptide of any of claims 128-129, wherein the peptide is a cyclic peptide.

131. The peptide of any of claims 128-130, wherein the peptide comprises 4 to 40 amino acids, optionally wherein the peptide comprises 6 to 10 amino acids.

132. The peptide of claim 131, wherein at least one amino acid of the peptide comprises the cyclic lactam group.

133. The peptide of any of claims 128-132, wherein the cyclic lactam group is an eight, nine, or ten-membered ring.

134. The peptide of any of claims 128-133, wherein the cyclic lactam group has a structure according to Formula III: 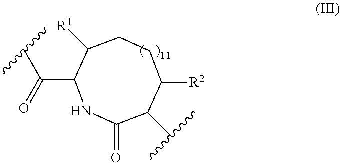
wherein:
R¹ and R² are each independently selected from the group consisting of H, an aryl group, a heteroaryl group, an alkylaryl group, an arylalkyl group, a linear alkyl group, a branched alkyl group, and a guanidine-comprising group, wherein each of R¹ and R² is optionally substituted with one or more substituents; and
n is an integer from 1 to 3.

135. The peptide of claim 134, wherein R¹ and/or R² independently comprise a substituted or unsubstituted aryl group.

136. The peptide of claim 135, wherein the aryl group is selected from the group consisting of a phenyl group, a benzyl group, and a naphthyl group.

137. The peptide of any of claims 134-136, wherein R¹ and/or R² independently comprise a substituted or unsubstituted guanidine-comprising group.

138. The peptide of claim 137, wherein the guanidine-comprising group is —(CH₂)₂CN₃H₄.

139. The peptide of any of claims 128-138, wherein the peptide comprises 1-5 arginine residues, optionally wherein the peptide comprises 2-4 arginine residues.

140. A cyclic peptide comprising at least one amino acid having a structure according to Formula IV: 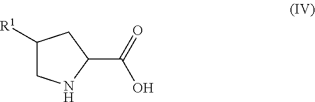
wherein R¹ comprises an aryl group or a guanidine-comprising group, wherein R¹ is optionally substituted with one or more substituents.

141. The cyclic peptide of claim 140, wherein the aryl group is selected from the group consisting of a benzyl group, a phenyl group, and a naphthyl group.

142. The cyclic peptide of claim 140, wherein the guanidine-comprising group is —(CH₂)₂—CN₃H₄.

143. A cyclic peptide comprising at least one oxadiazole linkage having a structure according to Formula V: 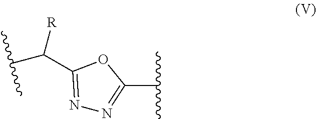
wherein R comprises a substituted or unsubstituted aryl group.

144. The cyclic peptide of claim 143, wherein the aryl group is selected from the group consisting of a phenyl group, a benzyl group, a naphthyl group, and a methyl naphthyl group.

145. The peptide-antisense oligonucleotide conjugate of any of claims 1-16 or 18-38, wherein the peptide-antisense oligonucleotide conjugate is a pharmaceutically acceptable salt.

146. The composition of claim 17, wherein the peptide-antisense oligonucleotide conjugate is a pharmaceutically acceptable salt.

147. The method of inducing Exon-2 skipping in the CD33 gene during pre-mRNA splicing of any of claims 39-58, wherein the peptide-antisense oligonucleotide conjugate is a pharmaceutically acceptable salt.

148. The method of treating a subject having a neurodegenerative disease of any of claims 59-78, wherein the peptide-antisense oligonucleotide conjugate is a pharmaceutically acceptable salt.

149. The peptide-antisense oligonucleotide conjugate for use in a method of inducing Exon-2 skipping in the CD33 gene during pre-mRNA splicing of any of claims 79-98, wherein the peptide-antisense oligonucleotide conjugate is a pharmaceutically acceptable salt.

150. The peptide-antisense oligonucleotide conjugate for use in a method of treating a subject having a neurodegenerative disease of any of claims 99-117, wherein the peptide-antisense oligonucleotide conjugate is a pharmaceutically acceptable salt.

151. The peptide of any of claims 118-139, wherein the peptide is a pharmaceutically acceptable salt.

152. The cyclic peptide of any of claims 140-144, wherein the cyclic peptide is a pharmaceutically acceptable salt.
